(19) United States
(12) Patent Application Publication
(10) Pub. No.: US 2025/0313858 A1
(43) Pub. Date: Oct. 9, 2025

(54) SYNTHETIC MODIFIED VACCINIA ANKARA (SMVA) BASED CORONAVIRUS VACCINES

(71) Applicant: CITY OF HOPE, Duarte, CA (US)

(72) Inventors: Don J. DIAMOND, Glendora, CA (US); Felix WUSSOW, Glendora, CA (US); Flavia CHIUPPESI, Duarte, CA (US)

(73) Assignee: CITY OF HOPE, Duarte, CA (US)

(21) Appl. No.: 19/183,257

(22) Filed: Apr. 18, 2025

Related U.S. Application Data
(63) Continuation of application No. 17/999,170, filed on Nov. 17, 2022.
(60) Provisional application No. 63/026,127, filed on May 17, 2020.
(60) Provisional application No. 63/044,033, filed on Jun. 25, 2020.
(60) Provisional application No. 63/113,810, filed on Nov. 13, 2020.
(60) Provisional application No. 63/161,371, filed on Mar. 15, 2021.

(51) Int. Cl. C12N 15/86; A61K 39/00; A61K 39/215; A61P 31/14; C07K 14/005

(52) U.S. Cl. CPC C12N 15/86; A61K 39/215; A61P 31/14; C07K 14/005; A61K 2039/53; A61K 2039/575; C07K 2319/02; C07K 2319/03; C12N 2710/24143; C12N 2710/24171; C12N 2770/20022; C12N 2770/20034; C12N 2770/20071

(57) ABSTRACT

Disclosed are synthetic MVA-based vaccines for preventing or treating coronavirus infections and methods of producing the vaccines.

### PRIORITY CLAIM

This application is a continuation of U.S. patent application Ser. No. 17/999,170, filed Nov. 17, 2022, which is a national stage application of International Patent Application No. PCT/US21/32821, filed May 17, 2021, which claims priority to U.S. Provisional Patent Application No. 63/026,127, filed May 17, 2020, U.S. Provisional Patent Application No. 63/044,033, filed Jun. 25, 2020, U.S. Provisional Patent Application No. 63/113,810, filed Nov. 13, 2020, and U.S. Provisional Patent Application No. 63/161,371, filed Mar. 15, 2021, the contents of which are hereby incorporated by reference in their entireties.

### SEQUENCE LISTING

This application contains a ST.26 compliant Sequence Listing, which was submitted in XML format via Patent Center, and is hereby incorporated by reference in its entirety. The XML copy, created on Feb. 24, 2025, is named 0544358206US06.xml and is 751,000 bytes in size.

### BACKGROUND

Modified Vaccinia Ankara (MVA) is a highly attenuated orthopoxvirus that was derived from its parental strain Chorioallantois Vaccinia Ankara (CVA) by 570 passages on chicken embryo fibroblasts (CEF). As a result of the attenuation process MVA has acquired six major genome deletions (Del1-6) as well as multiple shorter deletions, insertions, and point mutations, leading to gene fragmentation, truncation, short internal deletions, and amino acid substitutions. MVA has severely restricted host cell tropism, allowing productive assembly only in avian cells, e.g., CEF and baby hamster kidney (BHK) cells, whereas in human and most other mammalian cells, MVA assembly is abortive due to a late block in virus assembly. Although non-pathogenic and highly attenuated, MVA maintains excellent immunogenicity as demonstrated in various animal models and humans. In the late phase of the smallpox eradication campaign, MVA was used as a priming vector for the replication competent vaccinia-based vaccine in over 120,000 individuals in Germany and no adverse events were reported. In the past decades, MVA has been developed as a stand-alone smallpox vaccine and is currently pursued by the United States (US) government as a safer alternative to substitute the existing vaccinia-based vaccine stocks as a preventative countermeasure in case of a smallpox outbreak. The FDA approved MVA, under the trade name Jynneos (Bavarian Nordic) on Sep. 24, 2019 to prevent both smallpox and monkey pox. Previously, a similar MVA vaccine using the trade name Imvamune was approved in Europe as a smallpox vaccine. Almost all organizations that we are aware of which currently use MVA vectors or derivatives thereof are licensed or owned by academic, commercial, or governmental entities, which greatly restricts their use to commercially develop MVA-based vaccine vectors.

Coronaviruses are a large family of enveloped, positive-sense single stranded RNA viruses that can infect people and cause serious infections and even pandemics. Such highly infectious coronaviruses include, for example, MERS-CoV, SARS-CoV, and SARS-CoV-2. Since the recent outbreak of the novel severe acute respiratory syndrome coronavirus-2 (SARS-CoV-2, also known as Covid-19 or nCOV-2019) (PMC7095418, PMC7092803), the virus has spread to more than 200 countries, leading to over 3 million deaths worldwide. Although several effective SARS-CoV-2 vaccines have been developed with unprecedented pace and approved for emergency use, additional vaccines can contribute to establish long-term and cross-reactive immunity against SARS-CoV-2 and many of its emerging variants. Therefore, this disclosure provides vaccines using a synthetic MVA platform to satisfy an urgent need in the field.

### SUMMARY

In one aspect, disclosed herein is a vaccine composition for preventing or treating a virus infection such as coronavirus infection in a subject comprising: (i) a single DNA fragment comprising the entire genome of MVA, or two or more DNA fragments each comprising a partial sequence of the genome of the MVA such that the two or more DNA fragments, when expressed in the host cell upon co-transfection, are assembled sequentially and comprise the full-length sequence of the MVA genome, and (ii) one or more DNA sequences encoding one or more human coronavirus antigens, subunits, or fragments thereof inserted in one or more insertion sites of the MVA, wherein the antigens, subunits, or fragments thereof are expressed in the host cell upon transfection of the one or more DNA fragments. In certain embodiments, the DNA sequences of the antigens, subunits, or fragments thereof are codon optimized for expression in the host cell or vaccinia virus. In certain embodiments, the one or more human coronavirus antigens include the Spike(S) protein, the Nucleocapsid (N) protein, Membrane (M) protein, and Envelope (E) protein, papain-like protease, ORF1A, 3CL protease, ORF1B, endoribonuclease, matrix, helicase, or immunogenic fragments thereof. Other coronavirus antigens of the structural or non-structural (1a, 1b) proteins can be included as well. In certain embodiments, the one or more human coronavirus antigens include SARS-CoV-2 S protein, N protein, or both. In certain embodiments, the one or more antigens include a subunit of the S protein such as S1 and S2 domains, or the receptor-binding domain (RBD) of the S protein. In certain embodiments, the one or more antigens include a prefusion form of the S protein or N protein or a mutated S protein or N protein. For example, the prefusion form of the SARS-CoV-2 S protein can be stabilized or the SARS-CoV-2 S protein can be further stabilized by including a mutated Furin cleavage site such that amino acid residues 682-685 RRAR are mutated to GSAS. In another example, lysine 986 and valine 987 of the SARS-CoV-2 S protein are substituted with prolines (2P). Additional proline substitutions include F817P, A892P, A899P, and A942P. Similar Furin cleavage site mutations and proline substitutions can be included at the respective amino acid positions in other coronavirus S proteins to express uncleaved and/or 2P prefusion stabilized protein forms. In certain embodiments, the S protein comprises one or more of the mutations selected from the group consisting of S13I, L18F, T19R, T20N, R21T, P26S, a deletion of histidine, and valine at positions 69 and 70, K77T, D80A, T95I, D138Y, G142D, a deletion of tyrosine at position 144, W152C, E154K, a deletion of glutamic acid and phenylalanine at amino acid position 156 and 157 (Del156-157), R158G, R190S, D215G, Q218H, a deletion of leucine, alanine, and leucine at position 242-244, R246I, K417N, K417T, N439K, L452R, Y453F, S477N, T478K, E484K, E484Q, S494P, N501Y, S520S, A570D, D614G, H655Y, P681H, P681R, RRAR682-685GSAS, A701V, T716I, D950N, S982A, K986P, V987P, T1027I, Q1071H, H1101D, D1118H, and V1176F. In certain embodiments, the N protein comprises one or more of the mutations selected from the group consisting of D3L, P80R, S235F, R203K, R203M, G204R, T205I, and D377Y. In certain embodiments, the S protein and the N protein are fully mature or fully glycosylated. In certain embodiments, the S and N proteins are inserted in one or more MVA insertion sites. In certain embodiments, the one or more antigens comprise at least two RBDs from different variants of SARS-CoV-2, which can be linked by one or more GS linkers, and each of which can comprise a signal peptide at the N-terminus, or a transmembrane domain or a cytoplastic domain at the C-terminus. In certain embodiments, the one or more DNA fragments further comprise a virus promoter upstream of the DNA sequences encoding the human coronavirus antigens, subunits, or fragments thereof, a transcription termination signal downstream of the DNA sequences encoding the human coronavirus antigens, subunits, or fragments thereof, or both. In certain embodiments, the promoter sequences include mH5 and p7.5 promoters, or any other suitable native or synthetic vaccinia or poxvirus promoters. In certain embodiments, the DNA sequences encoding the antigens, subunits, or fragments thereof are inserted in one or more MVA insertion sites such as intergenic regions, non-essential genes and regions, and deletion sites. In certain embodiments, the vaccine composition further comprises a pharmaceutically acceptable carrier, adjuvant, additive or combination thereof. In certain embodiments, the subject is infected with or at a risk of being infected with a coronavirus such as a betacoronavirus, including MERS-CoV, SARS-COV and SARS-CoV2, 229E, NL63, OC43, HKU1, and other alpha, beta, gamma, and delta coronaviruses. In certain embodiments, the subject is infected with or at a risk of being infected with SARS-CoV-2.

In another aspect, disclosed herein is a method of preventing or treating a viral infection in a subject comprising administering a prophylactically or therapeutically effective amount of a vaccine composition to the subject, wherein the vaccine comprises: (i) a single DNA fragment comprising the entire genome of an MVA, or two or more DNA fragments each comprising a partial sequence of the genome of the MVA such that the two or more DNA fragments, when expressed in the host cell upon co-transfection, are assembled sequentially and comprise the full-length sequence of the MVA genome, and (ii) one or more DNA sequences encoding one or more human coronavirus antigens, subunits, or fragments thereof inserted in one or more insertion sites of the MVA, wherein the antigens, subunits, or fragments thereof are expressed in the host cell upon transfection of the one or more DNA fragments. In certain embodiments, the DNA sequences of the antigens, subunits, or fragments thereof are codon optimized for expression in the host cell or vaccinia virus. In certain embodiments, the one or more human coronavirus antigens include the Spike(S) protein, the Nucleocapsid (N) protein, Membrane (M) protein, and Envelope (E) protein, papain-like protease, ORF1A, 3CL protease, ORF1B, endoribonuclease, matrix, helicase, or immunogenic fragments thereof. Other coronavirus antigens of the structural or non-structural (1a, 1b) proteins can be included as well. In certain embodiments, the one or more human coronavirus antigens include SARS-CoV-2 S protein, N protein, or both. In certain embodiments, the one or more antigens include a subunit of the S protein such as S1 and S2 domains, or the receptor-binding domain (RBD) of the S protein. In certain embodiments, the one or more antigens include a prefusion form of the S protein and a mutated S protein. For example, the prefusion form of the SARS-CoV-2 S protein can be stabilized or the SARS-CoV-2 S protein can be further stabilized by including a mutated Furin cleavage site such that amino acid residues 682-685 RRAR are mutated to GSAS (RRAR682-685GSAS). In another example, lysine 986 and valine 987 of the SARS-CoV-2 S protein are substituted with prolines (2P) (K986P and V987P). Additional proline substitutions include F817P, A892P, A899P, and A942P. Similar Furin cleavage site mutations and proline substitutions can be included at the respective amino acid positions in other coronavirus S proteins to express uncleaved and/or 2P prefusion stabilized protein forms. In certain embodiments, the S protein comprises one or more of the mutations selected from the group consisting of S13I, L18F, T19R, T20N, R21T, P26S, a deletion of histidine, and valine at positions 69 and 70, K77T, D80A, T95I, D138Y, G142D, a deletion of tyrosine at position 144, W152C, E154K, a deletion of glutamic acid and phenylalanine at amino acid position 156 and 157 (Del156-157), R158G, R190S, D215G, Q218H, a deletion of leucine, alanine, and leucine at position 242-244, R246I, K417N, K417T, N439K, L452R, Y453F, S477N, T478K, E484K, E484Q, S494P, N501Y, S520S, A570D, D614G, H655Y, P681H, P681R, RRAR682-685GSAS, A701V, T716I, D950N, S982A, K986P, V987P, T1027I, Q1071H, H1101D, D1118H, and V1176F. In certain embodiments, the N protein comprises one or more of the mutations selected from the group consisting of D3L, P80R, S235F, R203K, R203M, G204R, T205I, and D377Y. In certain embodiments, the S protein and the N protein are fully mature or fully glycosylated. In certain embodiments, the one or more antigens comprise at least two RBDs from different variants of SARS-CoV-2, which can be linked by one or more GS linkers, and each of which can comprise a signal peptide at the N-terminus, or a transmembrane domain or a cytoplastic domain at the C-terminus. In certain embodiments, the S and N proteins are inserted in one or more MVA insertion sites. In certain embodiments, the one or more DNA fragments further comprise a virus promoter upstream of the DNA sequences encoding the human coronavirus antigens, subunits, or fragments thereof, a transcription termination signal downstream of the DNA sequences encoding the human coronavirus antigens, subunits, or fragments thereof, or both. In certain embodiments, the promoter sequences include mH5 and p7.5 promoters, or any other suitable native or synthetic vaccinia or poxvirus promoters. In certain embodiments, the DNA sequences encoding the antigens, subunits, or fragments thereof are inserted in one or more MVA insertion sites such as intergenic regions, non-essential genes and regions, and deletion sites. In certain embodiments, the vaccine composition further comprises a pharmaceutically acceptable carrier, adjuvant, additive or combination thereof. In certain embodiments, the subject is infected with or at a risk of being infected with a coronavirus such as a betacoronavirus, including MERS-CoV, SARS-COV and SARS-CoV2, 229E, NL63, OC43, HKU1, and other alpha, beta, gamma, and delta coronaviruses. In certain embodiments, the subject is infected with or at a risk of being infected with SARS-CoV-2.

In another aspect, disclosed herein is a method of eliciting an immune response in a subject comprising administering a prophylactically or therapeutically effective amount of a vaccine composition to the subject, wherein the vaccine comprises: (i) a single DNA fragment comprising the entire genome of an MVA, or two or more DNA fragments each comprising a partial sequence of the genome of the MVA such that the two or more DNA fragments, when expressed in the host cell upon co-transfection, are assembled sequentially and comprise the full-length sequence of the MVA genome, and (ii) one or more DNA sequences encoding one or more human coronavirus antigens, subunits, or fragments thereof inserted in one or more insertion sites of the MVA, wherein the antigens, subunits, or fragments thereof are expressed in the host cell upon transfection of the one or more DNA fragments. In certain embodiments, the DNA sequences of the antigens, subunits, or fragments thereof are codon optimized for expression in the host cell. In certain embodiments, the one or more human coronavirus antigens include the Spike(S) protein, the Nucleocapsid (N) protein, Membrane (M) protein, and Envelope (E) protein, papain-like protease, ORF1A, 3CL protease, ORF1B, endoribonuclease, matrix, helicase, or immunogenic fragments thereof. Other coronavirus antigens of the structural or non-structural (1a, 1b) proteins can be included as well. In certain embodiments, the one or more human coronavirus antigens include SARS-CoV-2 S protein, N protein, or both. In certain embodiments, the one or more antigens include a subunit of the S protein such as S1 and S2 domains, or the receptor-binding domain (RBD) of the S protein. In certain embodiments, the one or more antigens include a prefusion form of the S protein and a mutated S protein. For example, the prefusion form of the SARS-CoV-2 S protein can be stabilized or the SARS-CoV-2 S protein can be further stabilized by including a mutated Furin cleavage site such that amino acid residues 682-685 RRAR are mutated to GSAS. In another example, lysine 986 and valine 987 of the SARS-CoV-2 S protein are substituted with prolines (2P). Additional proline substitutions include F817P, A892P, A899P, and A942P. Similar Furin cleavage site mutations and proline substitutions can be included at the respective amino acid positions in other coronavirus S proteins to express uncleaved and/or 2P prefusion stabilized protein forms. In certain embodiments, the S protein comprises one or more of the mutations selected from the group consisting of S13I, L18F, T19R, T20N, R21T, P26S, a deletion of histidine, and valine at positions 69 and 70, K77T, D80A, T95I, D138Y, G142D, a deletion of tyrosine at position 144, W152C, E154K, a deletion of glutamic acid and phenylalanine at amino acid position 156 and 157 (Del156-157), R158G, R190S, D215G, Q218H, a deletion of leucine, alanine, and leucine at position 242-244, R246I, K417N, K417T, N439K, L452R, Y453F, S477N, T478K, E484K, E484Q, S494P, N501Y, S520S, A570D, D614G, H655Y, P681H, P681R, RRAR682-685GSAS, A701V, T716I, D950N, S982A, K986P, V987P, T1027I, Q1071H, H1101D, D1118H, and V1176F. In certain embodiments, the N protein comprises one or more of the mutations selected from the group consisting of D3L, P80R, S235F, R203K, R203M, G204R, T205I, and D377Y. In certain embodiments, the S protein and the N protein are fully mature or fully glycosylated. In certain embodiments, the one or more antigens comprise at least two RBDs from different variants of SARS-CoV-2, which can be linked by one or more GS linkers, and each of which can comprise a signal peptide at the N-terminus, or a transmembrane domain or a cytoplastic domain at the C-terminus. In certain embodiments, the S and N proteins are inserted in one or more MVA insertion sites. In certain embodiments, the one or more DNA fragments further comprise a virus promoter upstream of the DNA sequences encoding the human coronavirus antigens, subunits, or fragments thereof, a transcription termination signal downstream of the DNA sequences encoding the human coronavirus antigens, subunits, or fragments thereof, or both. In certain embodiments, the promoter sequences include mH5 and p7.5 promoters, or any other suitable native or synthetic vaccinia or poxvirus promoters. In certain embodiments, the DNA sequences encoding the antigens, subunits, or fragments thereof are inserted in one or more MVA insertion sites such as intergenic regions, non-essential genes and regions, and deletion sites. In certain embodiments, the vaccine composition further comprises a pharmaceutically acceptable carrier, adjuvant, additive or combination thereof. In certain embodiments, the subject is infected with or at a risk of being infected with a coronavirus such as a betacoronavirus, including MERS-CoV, SARS-COV and SARS-CoV2, 229E, NL63, OC43, HKU1, and other alpha, beta, gamma, and delta coronaviruses. In certain embodiments, the subject is infected with or at a risk of being infected with SARS-CoV-2.

In yet another aspect, this disclosure relates to a method of producing an MVA vector or a recombinant MVA vector. The method entails the steps of transfecting one or more DNA fragments into a host cell, wherein the one or more DNA fragments comprise the entire genomic DNA sequence of an MVA species, such that the MVA virus is reconstituted in the host cell. In certain embodiments, two or more DNA fragments are co-transfected into the host cell, each DNA fragment comprises a partial sequence of the MVA genome such that the two or more DNA fragments are assembled sequentially by homologous recombination and comprise the full-length sequence of the MVA genome when reconstituted in the host cell. In certain embodiments, the method further entails infecting the host cell with a helper virus before, during, or after the transfection of the one or more DNA fragments to initiate the transcription of the one or more DNA fragments. In certain embodiments, the helper virus is Fowl pox virus (FPV) or any other helper virus that stimulates MVA, vaccinia, or poxvirus transcription. In certain embodiments, the one or more DNA fragments are circularized before transfection or transfected in circular forms into the host cell. In certain embodiments, the one or more DNA fragments are cloned into a plasmid or a bacterial artificial chromosome (BAC) vector. In certain embodiments, the one or more DNA fragments are naturally derived, chemically synthesized, or a combination of naturally derived and chemically synthesized DNA fragments. In certain embodiments, the MVA genomic sequence comprises the sequence of Accession No. #U94848. In certain embodiments, two adjacent DNA fragments have an overlapping sequence to facilitate homologous recombination. In certain embodiments, the overlapping sequence is between about 100 bp and about 5000 bp in length. In certain embodiments, the one or more DNA fragments further comprise an inverted terminal repeat (ITR) region. In certain embodiments, the one or more DNA fragments further comprise an MVA terminal hairpin loop (HL) sequence, an MVA genome resolution (CR) sequence, or both, wherein the HL or the CR sequence is added to one or both ends of the DNA fragment as single stranded or double stranded DNA sequences in sense or antisense orientation. In certain embodiments, the one or more DNA fragments further comprise one or more HL sequences and one or more CR sequences. In certain embodiments, each HL sequence is flanked by two CR sequences at both ends of the HL sequence. In certain embodiments, the one or more DNA fragments further comprise one or more DNA sequences encoding one or more antigens, subunits, or fragments thereof. In certain embodiments, the DNA sequences of the antigens, subunits, or fragments thereof are codon optimized for expression in the host cell, e.g., in human cells or vaccinia virus, and/or codon optimized for stability in vaccinia by silent-codon alternation to avoid 4 or more of the same nucleotides consecutively. In certain embodiments, the one or more antigens include human coronavirus antigens such as the Spike(S) protein, the Nucleocapsid (N) protein, Membrane (M) protein, and Envelope (E) protein, papain-like protease, ORF1A, 3CL protease, ORF1B, endoribonuclease, matrix, helicase, or immunogenic fragments thereof. Other coronavirus antigens of the structural or non-structural (1a, 1b) proteins can be included as well. In certain embodiments, the one or more antigens include a subunit of the S protein such as S1 and S2 domains, or the receptor-binding domain (RBD) of the S protein. In certain embodiments, the one or more antigens include a prefusion form of the S protein and a mutated S protein. For example, the prefusion form of the SARS-CoV-2 S protein can be stabilized or the SARS-CoV-2 S protein can be further stabilized by including a mutated Furin cleavage site such that amino acid residues 682-685 RRAR are mutated to GSAS. In another example, lysine 986 and valine 987 of the SARS-CoV-2 S protein are substituted with prolines (2P). Additional proline substitutions include F817P, A892P, A899P, and A942P. Similar Furin cleavage site mutations and proline substitutions can be included at the respective amino acid positions in other coronavirus S proteins to express uncleaved and/or 2P prefusion stabilized protein forms. In certain embodiments, the S protein comprises one or more of the mutations selected from the group consisting of S13L, L18F, T19R, T20N, R21T, P26S, a deletion of histidine, and valine at positions 69 and 70, K77T, D80A, T95I, D138Y, G142D, a deletion of tyrosine at position 144, W152C, E154K, a deletion of glutamic acid and phenylalanine at amino acid position 156 and 157 (Del156-157), R158G, R190S, D215G, Q218H, a deletion of leucine, alanine, and leucine at position 242-244, R246I, K417N, K417T, N439K, L452R, Y453F, S477N, E484K, E484Q, S494P, N501Y, S520S, A570D, D614G, H655Y, P681H, P681R, RRAR682-685GSAS, A701V, T716I, D950N, S982A, K986P, V987P, T1027I, Q1071H, H1101D, D1118H, and V1176F. In certain embodiments, the N protein comprises one or more of the mutations selected from the group consisting of D3L, P80R, S235F, R203K, R203M, G204R, T205I, and D377Y. In certain embodiments, the S protein and the N protein are fully mature or fully glycosylated. In certain embodiments, the one or more DNA fragments further comprise a virus promoter upstream of the DNA sequences of the antigens, subunits, or fragments thereof, a transcription termination signal downstream the DNA sequences of the antigens, subunits, or fragments thereof, or both. In certain embodiments, the promoter sequences include mH5 and p7.5 promoters, or any other suitable native or synthetic vaccinia or poxvirus promoters. In certain embodiments, the DNA sequences encoding the antigens, subunits, or fragments thereof are inserted in one or more MVA insertion sites such as intergenic regions, non-essential genes and regions, and deletion sites. In another embodiment, the one or more expressed SARS-CoV-2 antigens are further modified to contain one or more mutations of emerging variants of concern (VOC).

### BRIEF DESCRIPTION OF THE DRAWINGS

FIGS. 1A-1F show sMVA construction and characterization. FIG. 1A: Schematic of MVA genome. The MVA genome is about 178 kbp in length and contains about 9.6 kbp inverted terminal repeat (ITR) sequences. FIG. 1B: sMVA fragments. The three sub-genomic sMVA fragments (F1-F3) comprise about 60 kbp of the left, central, and right part of the MVA genome as indicated. sMVA F1/F2 and F2/F3 share about 3 kbp overlapping homologous sequences for recombination (red dotted crossed lines). Approximate genome positions of commonly used MVA insertion (Del2, IGR69/70, Del3) are indicated. FIG. 1C: Terminal CR/HL/CR sequences. Each of the sMVA fragments contains at both ends a sequence composition comprising a duplex copy of the MVA terminal hairpin loop (HL) flanked by concatemeric resolution (CR) sequences. BAC=bacterial artificial chromosome vector. FIG. 1D: sMVA reconstitution. The sMVA fragments are isolated from the E. coli and co-transfected into BHK-21 cells, which are subsequently infected with FPV as a helper virus to initiate sMVA virus reconstitution. FIG. 1E: PCR analysis. CEF infected with sMVA, derived with FPV HP1.441 (sMVA hp) or TROVAC from two independent virus reconstitutions (sMVA tv1 and sMVA tv2), were investigated by PCR for several MVA genome positions (ITR sequences, transition left or right ITR into internal unique region (left ITR/UR; UR/right ITR), Del2, IGR69/70 and Del3 insertion sites, and F1/F2 and F2/F3 recombination sites) and absence of BAC vector sequences. PCR reactions with wtMVA-infected and uninfected cells, without sample (mock), or with MVA BAC were performed as controls. FIG. 1F: Restriction fragment length analysis. Viral DNA isolated from purified sMVA (sMVA tv1 and sMVA tv2) or wtMVA virus was compared by KpnI and XhoI restriction enzyme digestion.

FIGS. 2A-2D show sMVA replication properties. The replication properties of sMVA derived with FPV HP1.441 (sMVA hp) or TROVAC from two independent sMVA virus reconstitution (sMVA tv1 and sMVA tv2) were compared with wtMVA. FIG. 2A: Viral foci. CEF infected at low multiplicity of infection (MOI) with the reconstituted sMVA virus or wtMVA were immunostained using anti-Vaccinia polyclonal antibody (αVAC). FIG. 2B: Replication kinetics. BHK-21 or CEF cells were infected at 0.02 MOI with sMVA or wtMVA and viral titers of the inoculum and infected cells at 24 and 48 hours post infection were determined on CEF. Mixed-effects model with the Geisser-Greenhouse correction was applied; at 24 and 48 hours post-infection differences between groups were not significant. FIG. 2C: Viral foci size analysis. BHK-21 or CEF cell monolayers were infected at 0.002 MOI with sMVA or wtMVA and areas of viral foci were determined at 24 hours post infection following immunostaining with αVAC antibody. FIG. 2D: Host cell range analysis. Various human cell lines (HEK293, A549, 143b, and HeLa), CEF or BHK-21 cells were infected at 0.01 MOI with sMVA or wtMVA and virus titers were determined at 48 hours post infection on CEF. Dotted lines indicate the calculated virus titer of the inoculum based on 0.01 MOI. Differences between groups in FIGS. 2C-2D were calculated using one-way ANOVA followed by Tukey's (2C) or Dunnett's (2D) multiple comparison tests. ns=not significant.

FIGS. 3A-3D demonstrate sMVA in vivo immunogenicity. sMVA derived either with FPV HP1.441 (sMVA hp) or TROVAC from two independent virus reconstitution (sMVA tv1 and sMVA tv2) was compared by in vitro analysis with wtMVA. C57BL/6 mice were immunized twice at three-week interval with low (1×10⁷ PFU) or high (5×10⁷ PFU) dose of sMVA or wtMVA. Mock-immunized mice were used as controls. FIG. 3A: Binding antibodies. MVA-specific binding antibodies (IgG titer) stimulated by sMVA or wtMVA were measured after the first and second immunization by ELISA. FIG. 3B: NAb responses. MVA-specific NAb titers induced by sMVA or wtMVA were measured after the booster immunization against recombinant wtMVA expressing a GFP marker. FIGS. 3C-3D: T cell responses. MVA-specific IFNγ, TNFα, IL-4, and IL-10-secreting CD8+ (3C) and CD4+ (3D) T cell responses induced by sMVA or wtMVA after two immunizations were measured by flow cytometry following ex vivo antigen stimulation using B8R immunodominant peptides. Differences between groups were evaluated using one-way ANOVA with Tukey's multiple comparison test. ns=not significant.

FIGS. 4A-4D demonstrate sMVA immunogenicity in vivo. sMVA derived either with FPV strain HP1.441 (sMVA hp) or with FPV strain TROVAC from two independent virus reconstitution (sMVA tv1 and sMVA tv2) was compared by in vitro analysis with wtMVA. C57BL/6 mice (N=4) were immunized twice in a three-week interval with low (1×10⁷ PFU) or high (5×10⁷ PFU) dose of sMVA or wtMVA. Mock-immunized mice were used as controls. FIG. 4A: Binding antibodies. Shown is the absorbance at 450 nm at different serum dilutions of MVA-specific binding antibodies (IgG titer) measured by ELISA after the first and second immunization in mice receiving sMVA or wtMVA. FIG. 4B: NAb responses. MVA-specific NAb titers induced by sMVA or wtMVA were measured after the booster immunization against wtMVA expressing a GFP marker. Shown is the measured GFP area of infected cells in square pixels (pix²×10³) at different serum dilutions. FIGS. 4C-4D: T cell responses. MVA-specific CD8+ (4C) and CD4+ (4D) T cells expressing IFNγ, TNFα, IL-4, and IL-10 were measured after two immunizations with sMVA or wtMVA by flow cytometry following ex vivo antigen stimulation using Vaccinia A19L immunodominant peptides. Differences between groups were evaluated using one-way ANOVA with Tukey's multiple comparison test. ns=not significant.

FIGS. 5A-5E show construction and characterization of sMVA-CoV2 vectors. FIG. 5A: Schematic representation of vector construction. S and N antigen sequences (red spheres and green triangles) were inserted into sMVA fragments F2 and F3 by bacterial recombination methods in E. coli. The modified sMVA fragments of F1 and F2 with inserted antigen sequences and the unmodified sMVA fragment F1 were isolated from E. coli and co-transfected into FPV-infected BHK-21 cells to initiate virus reconstitution. FIG. 5B: Schematics of single (sMVA-S, sMVA-N) and double (sMVA-N/S, sMVA-S/N) recombinant sMVA-CoV2 vectors with S and N antigen sequences inserted into commonly used MVA insertion sites (Del2, IGR69/70, Del3). All antigens were expressed via the Vaccinia mH5 promoter. ITR represents inverted terminal repeat. FIG. 5C: PCR analysis. CEFs infected with the sMVA-CoV2 vectors were evaluated by PCR with primers specific for the Del2 and Del3 insertion sites harboring the N and S antigen sequences or primers specific for the F1/F2 and F2/F3 recombination sites. FIG. 5D: Western Blot. BHK-21 cells infected with the single and double recombinant sMVA-CoV2 vectors derived with FPV HP1.441 (sMVA-S/N hp, sMVA-N/S hp) or TROVAC (sMVA-S/N tv, sMVA-N/S tv, sMVA-S tv, sMVA-N tv) were evaluated for antigen expression by Western Blot using anti-S1 and N antibodies (αS1 and αN Abs). Vaccinia B5R protein was verified as infection control. Higher and lower molecular weight bands may represent mature and immature protein species. FIG. 5E: Flow cytometry staining. Hela cells infected with the vaccine vectors were evaluated by cell surface and intracellular flow staining using anti-S1, S2, and N antibodies (αS1, αS2, and αN Abs). Live cells were used to evaluate cell surface antigen expression. Fixed and permeabilized cells were used to evaluate intracellular antigen expression. Anti-Vaccinia virus antibody (αVAC) was used as staining control to verify MVA protein expression. Cells infected with sMVA or wtMVA or uninfected cells were used as controls for experiments in C, D and E as indicated. The experiments in C, D, and E were performed twice with similar results.

FIGS. 6A-6D show in vitro characterization of sMVA-CoV2 vectors. FIGS. 6A-6C: Immunofluorescence imaging. S and N antigen expression by the single (sMVA-S and sMVA-N) and double (sMVA-S/N and sMVA-N/S) recombinant vaccine sMVA-CoV2 vectors, all derived with FPV HP1.441, was evaluated in BHK-21 (6A and 6B) or HeLa (6C) cells by immunofluorescent confocal imaging using N and S-specific antibodies. Fluorescently-conjugated wheat germ agglutinin (WGA) was used in 6B and 6C to stain the cell membrane. Magnified insets are found below images. Scale bars in 6A, 50 μm. Scale bars in 6B and 6C, 10 μm. All images represent two independent experiments with similar results. FIG. 6D: Flow cytometry dual staining. Hela cells infected with the single (sMVA-N, sMVA-S) or double (sMVA-N/S, sMVA-S/N) recombinant sMVA-CoV2 vectors derived either with FPV TROVAC (tv) or HP1.441 (hp) were analyzed by intracellular flow cytometry analysis using dual staining with mouse anti-S2 and rabbit anti-N monoclonal antibodies followed by anti-mouse Alexa Fluor 488 and anti-rabbit Alexa Fluor 647. Percentage of cells dually stained by the S and N-specific antibodies is indicated in the upper-right quadrant (Q2).

FIGS. 7A-7H demonstrate humoral immune responses stimulated by sMVA-CoV2 vectors. Balb/c mice immunized twice in a three-week interval with 5×10⁷ PFU of the single and double recombinant sMVA-CoV2 vectors derived with FPV HP1.441 (sMVA-S/N hp and sMVA-N/S hp) or TROVAC (sMVA-S/N tv, sMVA-N/S tv, sMVA-S tv, sMVA-N tv) were evaluated for SARS-CoV-2-specific humoral immune responses. FIGS. 7A-7B: Binding antibodies. S, RBD, and N-specific binding antibodies induced by the vaccine vectors were evaluated after the first (7A) and second (7B) immunization by ELISA. Dashed lines in 7A and 7B indicate median binding antibody endpoint titers measured in convalescent human sera (FIG. 9). One-way ANOVA with Tukey's multiple comparison test was used to evaluate differences between binding antibody end-point titers. FIG. 7C: IgG2a/IgG1 isotype ratio. S-, RBD-, and N-specific binding antibodies of the IgG2a and IgG1 isotype were measured after the second immunization using 1:10,000 serum dilution, and absorbance reading was used to calculate IgG2a/IgG1 antibody ratio. One-way ANOVA with Dunnett's multiple comparison test was used to compare each group mean IgG2a/IgG1 ratio to a ratio of 1 (balanced Th1/Th2 response). FIGS. 7D-7G: NAb responses. SARS-CoV-2-specific NAb (NT90 titer) induced by the vaccine vectors were measured after the first (7D, 7F) and second (7E, 7G) immunization against SARS-CoV-2 pseudovirus (pv) (7D-7E) or infectious authentic SARS-CoV-2 virus (7F-7G) in pooled sera of immunized mice. Shown is the average NT90 measured in duplicate (7D-7E) or triplicate (7F-7G) infection. N/A=failed quality control of the samples. Dotted lines indicate lowest antibody dilution included in the analysis. FIG. 7H: SARS-CoV-2/SARS-CoV-2pv correlation analysis. Correlation analysis of NT90 measured in mouse sera after one and two immunizations using infectious SARS-CoV-2 virus and SARS-CoV-2pv. Pearson correlation coefficient (r) was calculated in H. *p<0.05. ns=not significant.

FIGS. 8A-8G demonstrate humoral immune responses induced by the sMVA-CoV2 vectors. Shown are the antibody measurements in Balb/c mice (N=5) immunized twice in a three week interval with 5×10⁷ PFU of the single or double recombinant sMVA-CoV2 vectors derived with FPV HP1.441 (sMVA-S/N hp and sMVA-N/S hp) or TROVAC (SMVA-S/N tv, sMVA-N/S tv, sMVA-S tv, sMVA-N tv). FIGS. 8A-8B: Binding antibodies. Shown are S-, RBD-, and N-specific ELISA measurements at 450 nm using serial dilutions of serum collected two weeks post-prime (8A) or one-week post-boost (8B). FIG. 8C: IgG2a/IgG1 isotype ratio. Binding antibodies of the IgG2a and IgG1 isotypes were measured in serum of mice post-boost using a dilution of 1:10,000. FIGS. 8D-8G: NAb responses. Shown is the percent (%) of SARS-CoV-2pv (8D-8E) and infectious authentic SARS-CoV-2 (8F-8G) neutralization measured in sera pooled from each group of immunized mice. Shown is the average % neutralization in duplicate (8D-8E) or triplicate (8F-8G) infection measured at different serum dilutions. Vaccine groups immunized with sMVA tv and PBS (mock) were not included in the analysis shown in 8G because of failure of quality control. Dotted lines mark 90% neutralization that was used to calculate NT90 in FIG. 7.

FIGS. 9A-9C demonstrate SARS-CoV-2-specific humoral immune responses in convalescent immune sera. S-, RBD, and N-specific binding antibodies were measured via ELISA using serial dilutions of plasma samples from SARS-CoV-2 convalescent individuals. FIG. 9A: Binding antibody curves from individual samples (N=19). FIG. 9B: SARS-CoV-2 convalescent plasma binding curves were grouped together and compared to binding measured in samples (N=2) from SARS-CoV-2 negative individuals. FIG. 9C: Endpoint binding antibody titers to S, RBD, and N were calculated in individual plasma samples. Lines represent the median endpoint titers. Due to the limited number of SARS-CoV-2-negative samples evaluated, statistical analysis was not performed.

FIGS. 10A-10B demonstrate humoral immune responses induced by sMVA-CoV2 vectors. C57BL/6 Nramp1 mice (N=5) were immunized twice in a three week interval with 5×10⁷ PFU of the single and double recombinant sMVA-CoV2 vectors derived with FPV HP1.441 (sMVA-S/N hp and sMVA-N/S hp) or TROVAC (sMVA-S/N tv, sMVA-N/S tv, sMVA-S tv, sMVA-N tv) and evaluated for SARS-CoV-2-specific humoral immune responses. FIGS. 10A-10B: Binding antibodies. S, RBD, and N-specific binding antibodies induced by the vaccine vectors were evaluated two weeks after the first immunization (10A) and one week after the second immunization (10B) by ELISA. Dashed lines in 10A and 10B indicate median binding antibody endpoint titers that were measured in convalescent human sera (FIG. 9). Data in 10A, and 10B is presented as mean values +/−SD. One-way ANOVA with Tukey's multiple comparison test was used to compare differences between binding antibody end-point titers in mice immunized with different vaccine vectors. FIG. 10C: IgG2c/IgG1 isotype ratio. S-, RBD-, and N-specific binding antibodies of the IgG2c and IgG1 isotype were measured after the second immunization using 1:10,000 serum dilution, and absorbance reading was used to calculate IgG2c/IgG1 antibody ratio. One-way ANOVA with Dunnett's multiple comparison test was used to compare each group mean IgG2c/IgG1 ratio to a ratio of 1 (balanced Th1/Th2 response). Lines represent median values. *0.05<p<0.01, **0.01<p<0.001, ***0.001<p<0.0001, ****p<0.0001. ns=not significant.

FIG. 11 shows that the immune sera of Balb/c mice immunized with 5×10\hat{ }7 PFU of the single and double recombinant sMVA-CoV2 vectors derived with FPV HP1.441 (SMVA-S/N hp and sMVA-N/S hp) or TROVAC (sMVA-S/N tv, sMVA-N/S tv, sMVA-S tv, sMVA-N tv) were evaluated for ADE effects. None of the sMVA-CoV2 vectors induced antibody-dependent enhancement (ADE) of infection, including the parental isolate of the clinical strain COH04S1, sMVA-N/S tv. Neutralizing (1:5,000) and non-neutralizing (1:50,000) dilutions (as assayed on stably-transduced HEK293T cells expressing ACE2 (HEK/ACE2)) were evaluated to promote THP-1 monocyte infection by SARS-CoV-2 pseudovirus (pv) expressing luciferase. VSV-G pv was used as infection control. Relative light units (RLU) were measured in duplicates at 48 hours post infection. Dotted lines represent the negative control (average relative light units (RLU) measured in cells in the absence of pv). Dashed lines represent the positive control (average RLU measured in cells in the absence of serum and in the presence of pv). 2-way ANOVA with Dunnett's multiple comparison test was used to compare each group and serum dilution to the mean RLU in the positive control. ns=not significant; * p<0.05.

FIGS. 12A-12D demonstrate cellular immune responses stimulated by sMVA-CoV2 vectors. Balb/c mice immunized twice in a three-week interval with 5×10⁷ PFU of the single or double recombinant sMVA-CoV2 vectors derived with FPV HP1.441 (sMVA-S/N hp and sMVA-N/S hp) or TROVAC (sMVA-S/N tv, sMVA-N/S tv, sMVA-S tv, sMVA-N tv) were evaluated for SARS-CoV-2-specific cellular immune responses. Antigen-specific CD8+ (12A and 12B) and CD4+ (12C and 12D) T cell responses induced by the vaccine vectors after two immunizations were evaluated by flow cytometry for IFNγ, TNFα, IL-4 and IL-10 secretion following ex vivo antigen stimulation using SARS-CoV-2 S and N-specific peptide libraries. Due to technical issues, 1-3 animals/group were not included in the CD4/TNFα analysis in 12C and 12D. One-way ANOVA with Tukey's multiple comparison test was used to compare differences in % of cytokine-specific T-cells between groups. *p<0.05. ns=not significant.

FIGS. 13A-13C show flow cytometry gating strategy. FIG. 13A: Intracellular staining analysis of mouse splenocytes stimulated with S and N peptide libraries was performed using a hierarchical gating strategy that included lymphocytes>singlets>CD3⁺ T-cells>CD4⁺ T-cells and CD8⁺ T-cells>Cytokine positive cells. FIGS. 13B-13C: Example of gating on cytokine-positive CD8⁺ T-cells (13B) and CD4⁺ T-cells (13C). Splenocytes of a mouse immunized with double recombinant sMVA-CoV2 vector sMVA-N/S were either left untreated (no peptide) or stimulated 16 hours with S or N peptide pools. Numbers in each dot plot indicate the percentage of cells in gated areas.

FIG. 14 shows TNFα secretion by T-cells of sMVA-CoV2-immunized mice. Splenocytes from Balb/c mice immunized with 5×10⁷ PFU of the single and double recombinant sMVA-CoV2 vectors derived with FPV HP1.441 (sMVA-S/N hp and sMVA-N/S hp) or TROVAC (sMVA-S/N tv, sMVA-N/S tv, sMVA-S tv, sMVA-N tv) were evaluated for TNFα secretion. Mouse splenocytes were stimulated with S or N peptide libraries and 48 hours later TNFα was measured by ELISA in cell culture supernatants. Amounts of TNFα quantified in unstimulated samples were subtracted from each peptide-stimulated sample. *p<0.05 compared to mock-immunized mice using one-way ANOVA with Dunnett's multiple comparison test.

FIGS. 15A-15E demonstrate humoral immune responses induced by sMVA-CoV2 vectors. SARS-CoV-2-specific humoral immune responses were evaluated in mice immunized with the single recombinant sMVA-CoV2 vectors sMVA-S and sMVA-N alone or in combination. Balb/c mice (N=5) were immunized twice in three-week interval with high (5×10⁷ PFU) or low (1×10⁷ PFU) dose of sMVA-S and sMVA-N. Co-immunization via the same immunization schedule with half of the high or low dose of each of the vaccine vectors was evaluated to assess SARS-CoV-2-specific immune stimulation to the S and N antigens by the vectors in combination. Mice immunized with empty sMVA vector or mock-immunized mice were used as controls. FIGS. 15A-15B: Binding antibodies. Antigen-specific binding antibodies to S, RBD, and N were determined after the first and second immunization by ELISA. Dashed lines indicate median binding antibody endpoint titers that were measured in convalescent human sera (FIG. 9). One-way ANOVA with Tukey's multiple comparison test was used to compare differences between binding antibody end-point titers in mice immunized with different vaccine doses, and mice immunized with the vaccine vectors alone or combined. FIG. 15C: IgG2a/IgG1 isotype ratio. Ratio of IgG2a/IgG1 binding antibodies to S, RBD, and N was calculated after performing isotype-specific ELISA for the different antigens using post-boost serum from immunized mice. One-way ANOVA with Dunnett's multiple comparison test was used to compare each group mean to a ratio of 1 (balanced Th1/Th2 response). FIGS. 15D-15E: NAb titers. SARS-CoV-2-specific NAb responses were measured after the second immunization in pooled sera by neutralization assay using SARS-CoV-2 pseudovirus. Shown in FIG. 15D are the neutralizing antibody titers to prevent 90% infection of SARS-CoV-2 pseudovirus (NT90). Dotted baseline represents the minimum dilution included in the analysis. Groups with NT90<baseline are shown at baseline. E shows % neutralization measured using serial dilutions of pooled sera. Dotted line in E marks 90% neutralization. * p<0.05.

FIGS. 16A-16B demonstrate cellular immune responses in vivo immunogenicity of sMVA-CoV2 vectors. SARS-CoV-2-specific cellular immune responses were evaluated in mice immunized with sMVA-CoV2 single recombinant vectors sMVA-S and sMVA-N alone or in combination. Balb/c mice (N=5) were immunized twice in three-week interval with high (5×10⁷ PFU) or low (1×10⁷ PFU) dose of sMVA-CoV2 single recombinant sMVA-S and sMVA-N. Co-immunization via the same immunization schedule with half of the high or low dose of each of the vaccine vectors was evaluated to assess SARS-CoV-2-specific immune stimulation to the S and N antigens by the vaccine vectors in combination. Mice immunized with empty sMVA vector or mock-immunized mice were used as controls. Antigen-specific CD8+ T cells expressing IFNγ, TNFα, and IL-2 and CD4+ T cell expressing IFNγ were evaluated by flow cytometry staining following ex vivo antigen stimulation using SARS-CoV-2-specific S and N peptide libraries. One-way ANOVA followed by Dunnett's multiple comparison test was used to compare each group mean to the mean in mock-immunized mice. * p<0.05. ns=not significant.

FIG. 17 illustrates SARS-CoV-2 clinical candidate vaccine COH04S1, a synthetic MVA viral vector-based vaccine expressing SARS-CoV-2 S and N antigens (SMVA-N/S). The gene sequences encoding the S and N antigens are inserted into the Del3 and Del2 insertions sites as indicated. mH5=modified Vaccinia H5 promotor; ITR=inverted terminal repeats; UR=internal unique region.

FIG. 18 demonstrates that C46 (a.k.a. sMVA-S/N) induced potent binding antibody responses against SARS-CoV-2 in preclinical rodent models. Dashed lines indicate median binding antibody endpoint titers measured in convalescent human sera. The green diamonds represent C46 (a.k.a. sMVA-S/N), which is the sMVA-CoV2 double recombinant vaccine expressing both Spike and Nucleocapsid antigens. The blue circles represent sMVA-S, which is the single recombinant sMVA with Spike antigen only. The red squares represent sMVA-N, which is the sMVA-CoV2 single recombinant with Nucleocapsid antigen only. The brown inverted triangles represent sMVA viral vector without SARS-CoV-2 antigens. The navy triangles represent mock vaccination.

FIG. 19 demonstrates clinical candidate COH04S1 induced potent cellular (T Cell) immune responses in preclinical rodent models. Spike- and Nucleocapsid-specific IFNγ, TNFα, and IL-4 CD8+ and CD4+ responses induced by COH04S1 (C35, sMVA-N/S) were compared to responses induced by an sMVA-CoV2 single recombinant vaccine expressing only Spike (sMVA-S), empty control sMVA, mock immunized animals, and mice immunized with Spike admixed to Alum. COH04S1 clinical candidate induced robust Spike- and Nucleocapsid T cell responses.

FIG. 20 demonstrates humoral and cellular responses in C35 (sMVA-N/S) vaccinated mice indicating a Th1-response. Humoral and cellular responses in BALB/c mice immunized with clinical candidate COH04S1 (sMVA-N/S) were compared to responses induced by an sMVA expressing only Spike (sMVA-S), empty control sMVA, mock immunized animals, and mice immunized with Spike admixed to Alum. Spike/Alum immunized animals develop Th2 responses following vaccination as shown by IgG1 biased antibody responses and IL-4 biased T cell responses. Clinical candidate COH04S1 (SMVA-N/S) immunized mice presented a Th1 shifted humoral and cellular response.

FIG. 21 demonstrates clinical candidate COH04S1 induced potent SARS-CoV-2 neutralizing antibody response in preclinical rodent models using live SARS-CoV-2 in a plaque reduction assay on HeLa-ACE2 cells. *NT50/90 is the dilution of the (antibody-containing) serum showing 50/90% neutralization of infection.

FIG. 22 demonstrates clinical candidate COH04S1 elicited potent SARS-CoV-2-specific neutralizing antibodies (NAb) in mice using authentic SARS-CoV-2 virus on susceptible cells (VeroE6). Clinical candidate COH04S1-primed and prime-boosted mice serum was analyzed for neutralization of live SARS-CoV-2 and compared to neutralization in a pool of 35 human plasma samples from individuals with mild-to-severe SARS-CoV-2 infection.

FIG. 23 shows that the SARS-CoV2 vaccines based on sMVA did not induce antibody-dependent enhancement (ADE) of infection.

FIG. 24 shows that COH04S1 induced strong immune responses in mice following intraperitoneal (IP) and intranasal (IN) vaccinations. Clinical candidate COH04S1 was used to immunize Balb/c mice IP or IN. Clinical candidate COH04S1-induced responses were compared to humoral and cellular responses induced by Spike and Nucleocapsid proteins admixed with Alum. Antibody responses post-prime and post-boost were evaluated by IgG and IgA RBD-ELISA and authentic SARS-CoV-2 virus neutralization assay. T cell responses were evaluated by IFNγ-ELISPOT after stimulation of splenocytes with S- and N-specific peptide libraries.

FIGS. 25A-25D show CD8+ T-cell responses induced by SARS-CoV2 sMVA construct sMVA-N/S in HLA transgenic mice. HLA-B*07:02 (B7) transgenic mice (n=4) were immunized twice in a 3-week interval with 1×10⁷ PFU of SARS-CoV2 sMVA construct SMVA-N/S or sMVA control vector (reconstituted with FPV TROVAC). B7 mice (n=3) were mock-immunized as additional control. Development of SARS-CoV-2-specific CD8+ T cells was evaluated one week post booster immunization. FIGS. 25A-25D show intracellular cytokine staining. Nucleocapsid- (25A and 25C) and Spike-specific (25B and 25D) CD8+ T cells were evaluated by intracellular cytokine staining for IFNγ, TNFα, and IL-4 secretion following ex vivo antigen stimulation by N and S peptide libraries, respectively. Panels 25A and 25B show the percentage of CD3+/CD8+ T cells secreting IFNγ, TNFα, or IL-4 following peptide stimulation. Panels 25C and 25D show relative frequencies of CD8+ T cells secreting one or more cytokines after peptide stimulation. Total percentage of cytokine-secreting cells within CD3+/CD8+ population is indicated under each pie chart. One-way ANOVA with Dunnett's multiple comparison test was used in 25A and 25B. Data in 25A and 25B are presented as mean values±SD; *0.05<p<0.01, **0.01<p<0.001, ***0.001<p <0.0001, ****p<0.0001; ns=not significant.

FIG. 26 shows T cell responses in HLA-B*07:02 (B7) transgenic mice immunized with clinical candidate COH04S1 (SMVA-N/S). ELISPOT analysis of IFNγ-secreting cells was performed following stimulation with S and N peptide libraries, S library sub-pools (1S1, 2S1, S2), and N26 peptide containing the HLA-B*07:02-restricted N-specific immunodominant epitope SPRWYFYYL (SEQ ID NO: 89). Two-way ANOVA with Dunnett's multiple comparison test was used. Data is presented as mean values±SD; *0.05<p<0.01, **0.01<p<0.001, ***0.001<p<0.0001, ****p<0.0001; ns=not significant.

FIG. 27 shows that aged mice developed comparable immune responses to young mice following prime-boost immunization with clinical candidate COH04S1. Clinical candidate COH04S1 was used to immunized young (8 weeks old), middle aged (40 weeks old), and aged (80 weeks old) C57BL/6 mice. Control animals were mice of similar age immunized with Spike and Nucleocapsid proteins admixed with Alum, and mock-immunized animals. Neutralizing antibodies were evaluated using SARS-CoV-2 Spike pseudovirus on HEK-293/Ace2 cells. T cell responses were measured using mouse IFNγ ELISPOT assay after stimulating mouse splenocytes with Spike peptide subpools (1S1, 2S1 and S2), and N peptides.

FIG. 28 shows the immunogenicity of clinical candidate COH04S1. COH04S1 shows comparable immunogenicity between sexes in Balb/C mice and demonstrates Th1 immunity compared to S/N/Alum to all antigens.

FIG. 29 illustrates the hamster clinical candidate COH04S1 vaccine study design.

FIG. 30 shows titers of binding antibodies induced by sMVA-SARS-CoV-2 vectors and clinical candidate COH04S1 clinical candidate in hamsters after intramuscular (IM) or intranasal (IN) immunization with 1×10\hat{ }8 pfu of sMVA-CoV2 vaccine constructs. The sMVA-CoV2 vaccines used in the study were: C79 (S2P/N double recombinant with 2P Spike sequence); clinical candidate COH04S1 (C35/F4/B1, double plaque purified isolate of C35 double recombinant); C35/F4/D5 (double plaque purified isolate of C35 double recombinant); C46/C3/F10 (double plaque purified isolate of C46 double recombinant); C15 (single recombinant expressing Spike only); C35 (double recombinant, and parental clone of COH04S1 clinical isolate). The sMVA empty vector was used as a control.

FIG. 31 shows neutralizing antibodies (PRNT) induced by sMVA-SARS-CoV-2 vectors and COH04S1 clinical candidate in hamsters measured at day 42. The hamsters were primed at day zero and boosted at day 28 with 1×10\hat{ }8 pfu of clinical candidate COH04S1 or sMVA-SARS-CoV-2 vectors intramuscularly or intranasally. Empty vector sMVA-immunized hamsters were used as a control. Antibodies neutralizing authentic SARS-CoV-2 virus were measured in vitro using Vero cells. PRNT assay was done at Bioqual (Gaithersburg, MD) using SARS-CoV-2, Isolate USA-WA1/2020. Day 42 serum samples were used for the analysis.

FIG. 32 shows body weight change of the hamsters which were immunized with sMVA-CoV2 vectors or COH04S1 and were challenged two weeks post-boost with 6×10\hat{ }4 pfu of authentic SARS-CoV-2 virus, Isolate USA-WA1/2020. The weight changes were measured daily for 10 days. All sMVA-CoV2 vectors including COH04S1 prevented severe weight loss in challenged animals.

FIG. 33 shows body weight change of the hamsters immunized with sMVA-CoV2 vectors either intramuscularly or intranasally and which were challenged two weeks post-boost with 6×10\hat{ }4 pfu of authentic SARS-CoV-2 virus, Isolate USA-WA1/2020. The weight changes were measured daily for 10 days. The animals were grouped by immunization route (top) or sex (bottom).

FIG. 34 shows binding antibodies and neutralizing antibodies induced by clinical candidate COH04S1 in hamsters. The hamsters were primed at day zero and boosted at day 28 with 1×10\hat{ }8 pfu of clinical candidate COH04S1 intramuscularly or intranasally. Empty vector sMVA-immunized hamsters were used as a control. Shown are endpoint binding antibody titers (total IgG) to Spike, RBD and Nucleocapsid measured in immunized hamsters post-prime (day 28) and post-boost (day 42). Ratios of IgG2/3 and IgG1 immunoglobulin titer to Spike, RBD, and Nucleocapsid are shown and are indicative of a Th1-biased response in clinical candidate COH04S1-immunized hamsters. The serum antibodies neutralizing authentic SARS-CoV-2 virus were measured in vitro using Vero cells. PRNT assay was done at Bioqual using SARS-CoV-2, Isolate USA-WA1/2020. Day 42 serum samples were used for the analysis.

FIG. 35 shows successful protection of clinical candidate COH04S1-vaccinated hamsters from sub-lethal challenge with authentic SARS-CoV-2 virus. The hamsters were challenged two weeks post-boost with SARS-CoV-2, Isolate USA-WA1/2020 and the weight changes were measured daily for 10 days. Thick lines indicate median weight loss values. Thin lines indicate single animals' weight loss.

FIGS. 36A-36B show a viral load analysis at day 10 post-challenge. The lungs and turbinates wash were collected 10 days post-challenge and analyzed for the presence of SARS-CoV-2 genomic RNA (gRNA) (FIG. 36A) and sub-genomic RNA (sgRNA, FIG. 36B). Clinical candidate COH04S1 given either intramuscularly (IM) or intranasally (IN) successfully prevented SARS-CoV-2 virus replication in lungs and reduced viral load in nasal turbinates.

FIG. 37 shows strong immune responses induced by intramuscular (IM) and intranasal (IN) vaccinations with clinical candidate COH04S1 in ferrets. Binding antibodies were evaluated using S-, RBD-, and N-IgG ELISA. Titers of neutralizing antibodies were measured using authentic SARS-CoV-2 virus on VeroE6 cells. T cell IFNγ responses in ferret′ PBMCs were measured using ferrets IFNγ-ELISPOT.

FIG. 38 illustrates the clinical candidate COH04S1 study design in African Green Monkeys (AGMs). The AGMs were immunized once or twice with clinical candidate COH04S1. In the prime-only study (Study 2) animals were immunized with 2.5×10\hat{ }8 pfu. In the prime-boost study (Study 1) the AGMs were immunized with 1×10\hat{ }8 pfu.

FIG. 39 shows that the T cell responses were evaluated in clinical candidate COH04S1-immunized AGMs. The levels of IFNγ T cells recognizing Spike(S) and Nucleocapsid (N) were quantified by ELISPOT following stimulation of freshly isolated PBMC with S and N peptide libraries.

FIG. 40 shows Study 1 (prime-boost) cellular responses. The IFNγ, IL-2 and IL-4 responses to Spike(S), Nucleocapsid (N) and Membrane (M) proteins were measured in freshly isolated PBMCs from prime-boosted AGMs 2 weeks and 5 weeks post-boost (time of challenge) by ELISPOT.

FIG. 41 shows Study 2 (prime-only) cellular responses. IFNγ, IL-2 and IL-4 responses to Spike(S), Nucleocapsid (N) and Membrane (M) proteins were measured in freshly isolated PBMC from primed AGM 2 weeks and 5 weeks post-prime (time of challenge) by ELISPOT.

FIG. 42 shows genomic RNA (gRNA) quantification in broncho alveolar lavage (BAL) by qPCR at day 2 and day 4 post-challenge.

FIG. 43 shows viral load quantification in broncho alveolar lavage (BAL) by TCID50 endpoint dilution assay at days 2, 4, and 7 post-challenge for Study 1 and Study 2.

FIG. 44 shows comparison BAL viral loads (TCID50) post-challenge in mock, sMVA and clinical candidate COH04S1 primed and primed-boosted AGMs.

FIG. 45A shows S-, RBD- and N-specific binding antibodies endpoint titers in DL1 sentinels up to day 120. FIG. 45B shows S-, RBD- and N-specific binding antibodies endpoint titers in DL2 sentinels up to day 90. FIG. 45C shows S-, RBD- and N-specific binding antibodies endpoint titers in DL3 sentinels up to day 56.

FIG. 46 shows binding antibodies endpoint titers against S, RBD, and N measured in clinical candidate COH04S1 DL1/DL2/DL3 sentinels up to day 120 post-prime. Serum from 11 individuals who received two doses of the EUA Pfizer-BioNTech SARS-CoV-2 mRNA EUA vaccine based on Spike was analyzed at days 60 and 90 post-vaccine, and antibody titers from a pool of 35 SARS-CoV-2 convalescent individuals who had mild-to-severe SARS-CoV-2 symptoms prior to sample collection were included as a comparison. DL1=4 sentinels d120, DL2-5 sentinels d90, DL3-6 sentinels d56, EUA=14 samples d60, 12 samples d90, convalescents=35 samples.

FIG. 47 shows comparison of binding antibodies to Spike (Wuhan D614G strain) and Spike P.1 Brazilian variant of concern (VOC) quantified by ELISA in DL1, DL2 and DL3 sentinels over time (top). Day 56-60 ELISA endpoint titers measured against Wuhan D614G Spike and P.1 Spike in DL1/DL2/DL3 sentinels and EUA Pfizer/BioNTech vaccine recipients (bottom).

FIG. 48 shows neutralizing antibody titers measured using Spike-pseudoviruses from SARS-CoV-2 Wuhan strain with D614G mutation (D614G) and variants of concern (VOC) B.1.1.7 (UK), B.1.351 (RSA) and P.1 (BRA) in DL1, DL2, and DL3 sentinels up to 90 days post-prime immunization.

FIG. 49 shows the neutralizing antibody titers measured using Spike-pseudoviruses from SARS-CoV-2 Wuhan strain with D614G mutation (D614G) and variants of concern (VOC) B.1.1.7 (UK), B.1.351 (RSA) and P.1 (BRA). DL1 and DL2 sentinels were evaluated at day 56, DL3 sentinels at day 42 or 56 when available, and EUA-Pfizer vaccine recipient at day 60.

FIG. 50 shows that healthy adults immunized with clinical candidate COH04S1 (DL1) developed functional T cell responses to S and N antigens in a preliminary analysis.

FIG. 51 shows the S-, N- and M-specific IFN-γ and IL-4 T cell responses measured in DL1 sentinels up to day 120, in DL2 sentinels up to day 90, and DL3 sentinels up to day 56 post-prime immunization with clinical candidate COH04S1 using an IFNγ/IL-4 fluorospot. PBMC were cultured in vitro for 48 hours in the presence of Spike, Nucleocapsid or Membrane peptide pools.

FIG. 52 shows that S-, N- and M-specific IFN-γ and IL-4 T cell responses measured in DL1, DL2 and DL3 sentinels up to 120 days post-prime immunization with clinical candidate COH04S1.

FIG. 53 shows that IFN-γ and IL-4 responses to S and N antigens measured in clinical candidate COH04S1 sentinels and in a pool of Pfizer/BioNTech vaccine recipients at days 56-60 (top) and 90 (bottom) post-prime immunization. COH04S1: d56/d90, EUA: d60/d90, and DO response was subtracted.

FIGS. 54A-54D show antigen expression by SARS-CoV-2 VOC vaccine vectors C163 and C164. CEF cells were infected with the VOC sMVA vectors C163 and C164 or the original COH04S1 sMVA-CoV2 vector (C35) and evaluated by Western Blot using antibodies specific for the S1 and S2 domains of the S protein (αS1 and αS2) or antibodies specific for N (αN). Uninfected CEF cells and CEF cells infected with empty sMVA vector were analyzed for control. Vaccinia B5R protein (αB5R) was detected to verify similar levels of infection between vaccine vectors.

FIGS. 55A-55C show antigen expression by SARS-CoV-2 VOC vaccine vector C170. CEF cells were infected with the VOC sMVA vector C170 or the original CIG04S1 sMVA vaccine construct (C35) and evaluated by Western Blot using antibodies specific for the S1 domain of the S protein (αS1) or antibodies specific for N (αN). Uninfected CEF cells and CEF cells infected with empty sMVA vector were analyzed as a control. Vaccinia B5R protein (αB5R) was detected to verify similar levels of infection between vaccine vectors.

FIG. 56 illustrates an overview of pre-clinical vaccine production process.

FIG. 57 is an extension of FIG. 56 and illustrates the derivation of clinical vaccine candidate COH04S1 from the original C35 sMVA-N/S vaccine vector.

FIG. 58 shows the sequence of sMVA-N/S (SEQ ID NO: 1) (deposited with NCBI under Accession No. MW036243, www.ncbi.nlm.nih.gov/nuccore/MW036243.1/).

FIG. 59 shows the sequence of sMVA-S/N (SEQ ID NO: 2) (deposited with NCBI under Accession No. MW030460, www.ncbi.nlm.nih.gov/nuccore/MW030460.1/).

FIG. 60 shows the DNA sequence/open reading frame (ORF) (5′ to 3′ end) of the Spike(S) antigen sequence based on the genome sequence of the NCBI SARS-CoV-2 reference strain (#NC_045512) (SEQ ID NO: 3), isolated Wuhan-Hu-1.

FIG. 61 shows the encoded protein sequence (N- to C-terminus) of the Spike(S) antigen sequence based on the genome sequence of the NCBI SARS-CoV-2 reference strain (#NC_045512) (SEQ ID NO: 4), isolated Wuhan-Hu-1.

FIG. 62 shows the DNA sequence/open reading frame (ORF) (5′ to 3′ end) of the SARS-CoV-2 S antigen sequence based on the Wuhan-Hu-1 reference strain (#NC_045512) (SEQ ID NO: 5) and optimized for stability in vaccinia by silent codon alteration to avoid 4 or more of the same nucleotides in consecutive order.

FIG. 63 shows the encoded protein sequence (N- to C-terminus) of the SARS-CoV-2 S antigen sequence based on the Wuhan-Hu-1 reference strain (#NC_045512) (SEQ ID NO: 6) and optimized for stability in vaccinia by silent codon alteration to avoid 4 or more of the same nucleotides in consecutive order.

FIG. 64 shows the DNA sequence/open reading frame (ORF) (5′ to 3′ end) of the Nucleocapsid (N) antigen sequence based on the genome sequence of the NCBI SARS-CoV-2 reference strain (#NC_045512) (SEQ ID NO: 7), isolate Wuhan-Hu-1.

FIG. 65 shows the encoded protein sequence (N- to C-terminus) of the Nucleocapsid (N) antigen sequence based on the genome sequence of the NCBI SARS-CoV-2 reference strain (#NC_045512) (SEQ ID NO: 8), isolate Wuhan-Hu-1.

FIG. 66 shows the DNA sequence/open reading frame (ORF) (5′ to 3′ end) of the SARS-CoV-2 N antigen sequence based on the Wuhan-Hu-1 reference strain (#NC_045512) (SEQ ID NO: 9) and optimized for stability in vaccinia by silent codon alteration to avoid 4 or more of the same nucleotides in consecutive order.

FIG. 67 shows the encoded protein sequence (N- to C-terminus) of the SARS-CoV-2 N antigen sequence based on the Wuhan-Hu-1 reference strain (#NC_045512) (SEQ ID NO: 10) and optimized for stability in vaccinia by silent codon alteration to avoid 4 or more of the same nucleotides in consecutive order.

FIG. 68 shows the DNA sequence/open reading frame (ORF) (5′ to 3′ end) of the codon-optimized S antigen sequence disclosed above (based on the Wuhan-Hu-1 reference strain (#NC_045512) and optimized for vaccinia) (SEQ ID NO: 11), which is further modified to encode for a prefusion stabilized S antigen with 2P alteration (lysine and valine at amino acid positions 986 and 987 substituted with prolines).

FIG. 69 shows the encoded protein sequence (N- to C-terminus) of the codon-optimized S antigen sequence disclosed above (based on the Wuhan-Hu-1 reference strain (#NC_045512) and optimized for vaccinia) (SEQ ID NO: 12), which is further modified to encode for a prefusion stabilized S antigen with 2P alteration (lysine and valine at amino acid positions 986 and 987 substituted with prolines).

FIG. 70 shows the DNA sequence/ORF (5′ to 3′ end) of the codon-optimized S antigen sequence disclosed above (based on the Wuhan-Hu-1 reference strain (#NC_045512) and optimized for vaccinia) (SEQ ID NO: 13), which is further modified to encode for a prefusion stabilized S antigen with 2P alteration (lysine and valine at amino acid positions 986 and 987 substituted with prolines) and mutated Furin cleavage site (RRAR amino acids at positions 682-685 substituted with GSAS).

FIG. 71 shows the encoded protein sequence (N- to C-terminus) of the codon-optimized S antigen sequence disclosed above (based on the Wuhan-Hu-1 reference strain (#NC_045512) and optimized for vaccinia) (SEQ ID NO: 14), which is further modified to encode for a prefusion stabilized S antigen with 2P alteration (lysine and valine at amino acid positions 986 and 987 substituted with prolines) and mutated Furin cleavage site (RRAR amino acids at positions 682-685 substituted with GSAS).

FIG. 72 shows the DNA sequence/ORF (5′ to 3′ end) of the codon-optimized S antigen sequence disclosed above (based on the Wuhan-Hu-1 reference strain (#NC_045512) and optimized for vaccinia) (SEQ ID NO: 15), which is further modified to encode for a prefusion stabilized S antigen with 2P alteration (lysine and valine at amino acid positions 986 and 987 substituted with prolines), mutated Furin cleavage site (RRAR amino acids at positions 682-685 substituted with GSAS), and 19 amino acid residues deleted at the C-terminus to prevent endoplasmic reticulum retention and to enhance cell surface expression.

FIG. 73 shows the encoded protein sequence (N- to C-terminus) of the codon-optimized S antigen sequence disclosed above (based on the Wuhan-Hu-1 reference strain (#NC_045512) and optimized for vaccinia) (SEQ ID NO: 16), which is further modified to encode for a prefusion stabilized S antigen with 2P alteration (lysine and valine at amino acid positions 986 and 987 substituted with prolines), mutated Furin cleavage site (RRAR amino acids at positions 682-685 substituted with GSAS), and 19 amino acid residues deleted at the C-terminus to prevent endoplasmic reticulum retention and to enhance cell surface expression.

FIG. 74 shows a SARS-CoV-2 S antigen sequence (SEQ ID NO: 17) which is fully codon-optimized for human expression, additionally optimized for stability in vaccinia by silent codon alteration to avoid 4 or more of the same nucleotides in consecutive order, and encodes for an S antigen with mutated Furin cleavage site and stabilizing 2P mutation.

FIG. 75 shows a SARS-CoV-2 S antigen sequence (SEQ ID NO: 18) which is fully codon-optimized for vaccinia virus expression, additionally optimized for stability in vaccinia by silent codon alteration to avoid 4 or more of the same nucleotides in consecutive order, and encodes for an S antigen with mutated Furin cleavage site and stabilizing 2P mutation.

FIG. 76 shows a SARS-CoV-2 N antigen sequence (SEQ ID NO: 19) which is fully codon-optimized for human expression and additionally optimized for stability in vaccinia by silent codon alteration to avoid 4 or more of the same nucleotides in consecutive order.

FIG. 77 shows a SARS-CoV-2 N antigen sequence (SEQ ID NO: 20) which is fully codon-optimized for vaccinia virus expression and additionally optimized for stability in vaccinia by silent codon alteration to avoid 4 or more of the same nucleotides in consecutive order.

FIG. 78 shows the DNA sequence/ORF (5′ to 3′ end) of the S1 domain encompassing 698 amino acid residues of the N-terminus of the SARS-CoV-2 S antigen based on the Wuhan reference strain (#NC_045512) (SEQ ID NO: 21) and optimized for stability in vaccinia.

FIG. 79 shows the encoded protein sequence (N- to C-terminus) of the S1 domain encompassing 698 amino acid residues of the N-terminus of the SARS-CoV-2 S antigen based on the Wuhan reference strain (#NC_045512) (SEQ ID NO: 22) and optimized for stability in vaccinia.

FIG. 80 shows the DNA sequence/ORF (5′ to 3′ end) of the S1 domain encompassing 680 amino acid residues of the N-terminus of the SARS-CoV-2 S antigen based on the Wuhan reference strain (#NC_045512) (SEQ ID NO: 23) and optimized for stability in vaccinia.

FIG. 81 shows the encoded protein sequence (N- to C-terminus) of the S1 domain encompassing 680 amino acid residues of the N-terminus of the SARS-CoV-2 S antigen based on the Wuhan reference strain (#NC_045512) (SEQ ID NO: 24) and optimized for stability in vaccinia.

FIG. 82 shows the DNA sequence/ORF (5′ to 3′ end) of an RBD encompassing amino acid residues 331-524 of the SARS-CoV-2 S antigen based on the Wuhan reference strain (#NC_045512) (SEQ ID NO: 25) and optimized for stability in vaccinia fused to the signal peptide of the S antigen (C-terminal 13 amino acids).

FIG. 83 shows the encoded protein sequence (N- to C-terminus) of an RBD encompassing amino acid residues 331-524 of the SARS-CoV-2 S antigen based on the Wuhan reference strain (#NC_045512) (SEQ ID NO: 26) and optimized for stability in vaccinia fused to the signal peptide of the S antigen (C-terminal 13 amino acids).

FIG. 84 shows the DNA sequence/ORF (5′ to 3′ end) of an RBD encompassing amino acid residues 319-541 of the SARS-CoV-2 S antigen based on the Wuhan reference strain (#NC_045512) (SEQ ID NO: 27) and optimized for stability in vaccinia fused to the signal peptide of the S antigen (C-terminal 13 amino acids).

FIG. 85 shows the encoded protein sequence (N- to C-terminus) of an RBD encompassing amino acid residues 319-541 of the SARS-CoV-2 S antigen based on the Wuhan reference strain (#NC_045512) (SEQ ID NO: 28) and optimized for stability in vaccinia fused to the signal peptide of the S antigen (C-terminal 13 amino acids).

FIG. 86 shows the DNA sequence/ORF (5′ to 3′ end) of the codon-optimized SARS-CoV-2 S antigen sequence based on the Wuhan-Hu-1 reference strain (#NC_045512) and optimized for vaccinia (SEQ ID NO: 29), which is further modified to encode for an S antigen that includes mutations of the B.1.351 variant lineage identified in South Africa (N501Y, E484K, K417N, L18F, D80A, D215G, Del242-244, R246I, D614G, A701I).

FIG. 87 shows the encoded protein sequence (N- to C-terminus) of the codon-optimized SARS-CoV-2 S antigen sequence based on the Wuhan-Hu-1 reference strain (#NC_045512) (SEQ ID NO: 30) and optimized for vaccinia, which is further modified to encode for an S antigen that includes mutations of the B.1.351 variant lineage identified in South Africa (N501Y, E484K, K417N, L18F, D80A, D215G, Del242-244, R246I, D614G, A701V).

FIG. 88 shows the DNA sequence/ORF (5′ to 3′ end) of the codon-optimized SARS-CoV-2 S antigen sequence based on the Wuhan-Hu-1 reference strain (#NC_045512) (SEQ ID NO: 31) and optimized for vaccinia, which is further modified to encode for an S antigen that includes mutations of the B.1.1.7 variant lineage identified in the UK (N501Y, Del69/70, Del144, A570D, D614G, P681H, T716I, S982A, D1118H).

FIG. 89 shows the encoded protein sequence (N- to C-terminus) of the codon-optimized SARS-CoV-2 S antigen sequence based on the Wuhan-Hu-1 reference strain (#NC_045512) (SEQ ID NO: 32) and optimized for vaccinia, which is further modified to encode for an S antigen that includes mutations of the B.1.1.7 variant lineage identified in the UK (N501Y, Del69/70, Del144, A570D, D614G, P681H, T716I, S982A, D1118H).

FIG. 90 shows the DNA sequence/ORF (5′ to 3′ end) of the codon-optimized SARS-CoV-2 S antigen sequence based on the Wuhan-Hu-1 reference strain (#NC_045512) (SEQ ID NO: 33) and optimized for vaccinia, which is further modified to encode for an S antigen that includes mutations of the B.1.429+B.1.427 variant lineage identified in California (D614G, L452R, S13I, W152C).

FIG. 91 shows the encoded protein sequence (N- to C-terminus) of the codon-optimized SARS-CoV-2 S antigen sequence based on the Wuhan-Hu-1 reference strain (#NC_045512) (SEQ ID NO: 34) and optimized for vaccinia, which is further modified to encode for an S antigen that includes mutations of the B.1.429+B.1.427 variant lineage identified in California (D614G, L452R, S13I, W152C).

FIG. 92 shows the DNA sequence/ORF (5′ to 3′ end) of the codon-optimized SARS-CoV-2 S antigen sequence based on the Wuhan-Hu-1 reference strain (#NC_045512) (SEQ ID NO: 35) and optimized for vaccinia, which is further modified to encode for an S antigen that includes mutations of P.1 variant lineage identified in Brazil (N501Y, E484K, K417T, L18F, T20N, P26S, D138Y, R190S, H655Y, T1027I, V1176F).

FIG. 93 shows the encoded protein sequence (N- to C-terminus) of the codon-optimized SARS-CoV-2 S antigen sequence based on the Wuhan-Hu-1 reference strain (#NC_045512) (SEQ ID NO: 36) and optimized for vaccinia, which is further modified to encode for an S antigen that includes mutations of P.1 variant lineage identified in Brazil (N501Y, E484K, K417T, L18F, T20N, P26S, D138Y, R190S, H655Y, T1027I, V1176F).

FIG. 94 shows the sequence encoding the S antigen of the Wuhan-Hu-1 reference strain (SEQ ID NO: 37).

FIG. 95 shows the sequence encoding the S antigen of the South African variant B.1.351 (SEQ ID NO: 38).

FIG. 96 shows the sequence encoding the S antigen of the UK variant B.1.1.7 (SEQ ID NO: 39).

FIG. 97 shows the DNA sequence/ORF (5′ to 3′ end) (SEQ ID NO: 40) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a triple polycistronic expression construct which comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42).

FIG. 98 shows the encoded protein sequence (N- to C-terminus) (SEQ ID NO: 43) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a triple polycistronic expression construct which comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42).

FIG. 99 shows the DNA sequence/ORF (5′ to 3′ end) (SEQ ID NO: 44) of the RBD domain of the Wuhan-Hu-1 reference strain, the RBD domain of the B.1.351 variant, and a RBD domain combining the N501Y and L452R mutations of the B.1.1.7 and B.1.429+B.1.427 variants co-expressed by a triple polycistronic expression construct comprising at the N-terminus a signal peptide of the S antigen (MFVFLVLLPLVSSQCV, SEQ ID NO: 41) and at the C-terminus a T4 Foldon domain (GYIPEAPRDGQAYVRKDGEWVLLSTFL, SEQ ID NO: 45) and in which the RBD domains are connected through GS linkers such as GSGSGS (SEQ ID NO: 42).

FIG. 100 shows the encoded protein sequence (N- to C-terminus) (SEQ ID NO: 46) of the RBD domain of the Wuhan-Hu-1 reference strain, the RBD domain of the B.1.351 variant, and a RBD domain combining the N501Y and L452R mutations of the B.1.1.7 and B.1.429+B.1.427 variants co-expressed by a triple polycistronic expression construct comprising at the N-terminus a signal peptide of the S antigen (MFVFLVLLPLVSSQCV, SEQ ID NO: 41) and at the C-terminus a T4 Foldon domain (GYIPEAPRDGQAYVRKDGEWVLLSTFL, SEQ ID NO: 45) and in which the RBD domains are connected through GS linkers such as GSGSGS (SEQ ID NO: 42).

FIG. 101 shows the DNA sequence/ORF (5′ to 3′ end) (SEQ ID NO: 47) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct in which each RBD domain comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42) and P2A and T2A peptides.

FIG. 102 shows the encoded protein sequence (N- to C-terminus) (SEQ ID NO: 48) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct in which each RBD domain comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42) and P2A and T2A peptides.

FIG. 103 shows the DNA sequence/ORF (5′ to 3′ end) (SEQ ID NO: 49) of the RBD domain of the Wuhan-Hu-1 reference strain, the RBD domain of the B.1.351 variant, and a RBD domain combining N501Y and L452R mutations of the B.1.1.7 and B.1.429+B.1.427 variants co-expressed by a polycistronic expression construct in which the RBD domains are connected through GS linkers (GSGSGS, SEQ ID NO: 42) and P2A and T2A peptides and in which each RBD domain is fused at the N-terminus to a S signal peptide (MFVFLVLLPLVSSQCV, SEQ ID NO: 41) and at the C-terminus to a T4 Foldon domain (GYIPEAPRDGQAYVRKDGEWVLLSTFL, SEQ ID NO: 45).

FIG. 104 shows the encoded protein sequence (N- to C-terminus) (SEQ ID NO: 50) of the RBD domain of the Wuhan-Hu-1 reference strain, the RBD domain of the B.1.351 variant, and a RBD domain combining N501Y and L452R mutations of the B.1.1.7 and B.1.429+B.1.427 variants co-expressed by a polycistronic expression construct in which the RBD domains are connected through GS linkers (GSGSGS, SEQ ID NO: 42) and P2A and T2A peptides and in which each RBD domain is fused at the N-terminus to a S signal peptide (MFVFLVLLPLVSSQCV, SEQ ID NO: 41) and at the C-terminus to a T4 Foldon domain (GYIPEAPRDGQAYVRKDGEWVLLSTFL, SEQ ID NO: 45).

FIG. 105 shows the DNA sequence/ORF (5′ to 3′ end) (SEQ ID NO: 51) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct comprising at the N-terminus a signal peptide of the S antigen (MFVFLVLLPLVSSQCV, SEQ ID NO: 41) and at the C-terminus TM and CT domains (without the last 19 amino acids) of the S antigen and in which the RBD domains are connected through GS linkers such as GSGSGS (SEQ ID NO: 42).

FIG. 106 shows the encoded protein sequence (N- to C-terminus) (SEQ ID NO: 52) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct comprising at the N-terminus a signal peptide of the S antigen (MFVFLVLLPLVSSQCV, SEQ ID NO: 41) and at the C-terminus TM and CT domains (without the last 19 amino acids) of the S antigen and in which the RBD domains are connected through GS linkers such as GSGSGS (SEQ ID NO: 42).

FIG. 107 shows the DNA sequence/ORF (5′ to 3′ end) (SEQ ID NO: 53) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct in which each of the RBD domains comprises at the N-terminus a signal peptide of the S protein (MFVFLVLLPLVSSQCV, SEQ ID NO: 41) and at the C-terminus TM and CT domains (without the last 19 amino acids) of the S antigen and in which the RBD domains are connected through GS linkers such as GSGSGS (SEQ ID NO: 42) and P2A and T2A peptide sequences.

FIG. 108 shows the encoded protein sequence (N- to C-terminus) (SEQ ID NO: 54) of the RBD domain of the Wuhan-Hu-1 reference strain, the RBD domain of the B.1.351 variant, and an RBD domain combining N501Y and L452R mutations of the B.1.1.7 and B.1.429+B.1.427 variants co-expressed by a polycistronic expression construct in which each of the RBD domains comprises at the N-terminus a signal peptide of the S protein (MFVFLVLLPLVSSQCV, SEQ ID NO: 41) and at the C-terminus TM and CT domains (without the last 19 amino acids) of the S antigen and in which the RBD domains are connected through GS linkers such as GSGSGS (SEQ ID NO: 42) and P2A and T2A peptide sequences.

FIG. 109 shows the DNA sequence/ORF (5′ to 3′ end) of a codon-optimized N antigen that includes the N-specific mutations in the B.1.1.7 variant lineage identified in the UK (SEQ ID NO: 55), including an aspartic acid to leucine substitution at amino acid position 3 of the N protein (D3L), a serine to phenylalanine substitution at amino acid position 235 of the N protein (S235F), an arginine to lysine substitution at amino acid position 203 of the N protein (R203K), and a glycine to arginine substitution at amino acid position 204 of the N protein (G204R).

FIG. 110 shows the encoded protein sequence (N- to C-terminus) of a codon-optimized N antigen that includes the N-specific mutations in the B.1.1.7 variant lineage identified in the UK (SEQ ID NO: 56), including an aspartic acid to leucine substitution at amino acid position 3 of the N protein (D3L), a serine to phenylalanine substitution at amino acid position 235 of the N protein (S235F), an arginine to lysine substitution at amino acid position 203 of the N protein (R203K), and a glycine to arginine substitution at amino acid position 204 of the N protein (G204R).

FIG. 111 shows the DNA sequence/ORF (5′ to 3′ end) of a codon-optimized N antigen that includes the N-specific mutations in the B.1.351 variant lineage identified in South Africa (SEQ ID NO: 57), including a threonine to isoleucine substitution at amino acid position 205 of the N protein (T205I).

FIG. 112 shows the encoded protein sequence (N- to C-terminus) of a codon-optimized N antigen that includes the N-specific mutations in the B.1.351 variant lineage identified in South Africa (SEQ ID NO: 58), including a threonine to isoleucine substitution at amino acid position 205 of the N protein (T205I).

FIG. 113 shows the DNA sequence/ORF (5′ to 3′ end) of a codon-optimized N antigen that includes the N-specific mutations in the P.1 variant lineage identified in Brazil (SEQ ID NO: 59), including a proline to arginine substitution at amino acid position 80 of the N protein (P80R), as well as R203K and G204R.

FIG. 114 shows the encoded protein sequence (N- to C-terminus) of a codon-optimized N antigen that includes the N-specific mutations in the P.1 variant lineage identified in Brazil (SEQ ID NO: 60), including a proline to arginine substitution at amino acid position 80 of the N protein (P80R), as well as R203K and G204R.

FIG. 115 shows the DNA sequence/ORF (5′ to 3′ end) of the codon-optimized SARS-CoV-2 S antigen sequence based on the Wuhan-Hu-1 reference strain (#NC_045512) (SEQ ID NO: 61) and optimized for vaccinia, which is further modified to encode for an S antigen that includes mutations of the B.1.617 variant lineage identified in India, including L452R and E484Q mutations in the RBD domain, D614G, a glycine to aspartic acid substitution at amino acid position 142 of the S protein (G142D), a glutamic acid to lysine substitution at amino acid position 154 of the S protein (E154K), a proline to lysine substitution at amino acid position 681 of the S protein (P681R), a glutamine to histidine substitution at amino acid position 1071 of the S protein (Q1071H), and a histidine aspartic acid substitution at amino acid position 1101 of the S protein (H1101D).

FIG. 116 shows the encoded protein sequence (N- to C-terminus) of the codon-optimized SARS-CoV-2 S antigen sequence (SEQ ID NO: 62) based on the Wuhan-Hu-1 reference strain (#NC_045512) and optimized for vaccinia, which is further modified to encode for an S antigen that includes mutations of the B.1.617 variant lineage identified in India, including L452R and E484Q mutations in the RBD domain, D614G, a glycine to aspartic acid substitution at amino acid position 142 of the S protein (G142D), a glutamic acid to lysine substitution at amino acid position 154 of the S protein (E154K), a proline to lysine substitution at amino acid position 681 of the S protein (P681R), a glutamine to histidine substitution at amino acid position 1071 of the S protein (Q1071H), and a histidine aspartic acid substitution at amino acid position 1101 of the S protein (H1101D).

FIG. 117 shows the DNA sequence/ORF (5′ to 3′ end) of the codon-optimized SARS-CoV-2 N antigen sequence encoding for an N antigen that includes the N-specific mutations in the B.1.617 variant lineage identified in India (SEQ ID NO: 63), including an arginine to methionine substitution at amino acid position 203 of the N protein (R203M) and an aspartic acid to tyrosine substitution at amino acid position 377 of the N protein (D377Y).

FIG. 118 shows the encoded protein sequence (N- to C-terminus) of the codon-optimized SARS-CoV-2 N antigen sequence encoding for an N antigen that includes the N-specific mutations in the B.1.617 variant lineage identified in India (SEQ ID NO: 64), including an arginine to methionine substitution at amino acid position 203 of the N protein (R203M) and an aspartic acid to tyrosine substitution at amino acid position 377 of the N protein (D377Y).

### DETAILED DESCRIPTION

Disclosed herein are methods of producing recombinant sMVA (rsMVA) expressing one or more heterologous gene sequences encoding coronavirus antigens. A fully synthetic version of MVA (sMVA) from circularized or linear synthetic DNA fragments is produced and disclosed in PCT application No. PCT/US21/16247, the content of which is incorporated by reference in its entirety. The sMVA or the rsMVA can be used as a vaccine for preventing and treating various conditions such as coronavirus infections and associated diseases.

Since the outbreak of the novel severe acute respiratory syndrome coronavirus-2 (SARS-CoV-2) in December 2019, the virus has spread to more than 200 countries worldwide, causing a pandemic of global magnitude with over 3 million deaths. Many vaccine candidates are currently under rapid development to control this global pandemic, some of which have entered with unprecedented pace into clinical trials. Most of these approaches employ antigenic forms of the Spike (S) protein as it is considered the primary target of protective immunity^{16,20-22}. The S protein mediates SARS-CoV-2 entry into a host cell through binding to angiotensin-converting enzyme 2 (ACE) and is the major target of neutralizing antibodies (NAb)²³⁻²⁵. Studies in rhesus macaques show that vaccine strategies based on the S antigen can prevent SARS-CoV-2 infection and disease in this relevant animal model¹⁸, indicating that the S antigen may be sufficient as vaccine immunogen to elicit protective immunity. However, a recent study showed that even patients without measurable NAb can recover from SARS-CoV-2 infection, suggesting that protection against SARS-CoV-2 infection is mediated by both humoral and cellular immunity to multiple immunodominant antigens, including S and Nucleocapsid (N) antigens^{20,26}. In this disclosure, the terms of “S protein” and “S antigen” are used interchangeably, and the terms of “N protein” and “N antigen” are used interchangeably.

Disclosed herein is a novel vaccine platform based on a uniquely designed three-plasmid system to efficiently generate recombinant MVA vectors from chemically synthesized DNA. In response to the ongoing global SARS-CoV-2 pandemic, this novel vaccine platform can be used to rapidly produce sMVA vectors co-expressing SARS-CoV-2 S and N antigens or any additional antigens. These antigens used for vaccine production can be based on the Wuhan reference strain or include one or more mutations based on emerging VOCs. As demonstrated in the working examples, these sMVA vectors induced potent SARS-CoV-2 antigen-specific humoral and cellular immunity in mice, including potent NAb. These results highlight the feasibility to efficiently produce recombinant MVA vectors from chemically synthesized DNA and to rapidly develop a synthetic poxvirus-based vaccine candidate to prevent SARS-CoV-2 infection.

Disclosed herein is a synthetic form of MVA and a method of producing the same using chemically synthesized DNA, which differs from the recently described approach to produce a synthetic horsepox virus vaccine vector⁴². In certain embodiments, a single DNA fragment is derived from viral DNA or chemically synthesized and comprises the entire genome sequence of MVA. This single DNA fragment can be used to transfect a host cell such that the MVA is reconstituted. In other embodiments, two or more naturally derived or chemically synthesized DNA fragments, or a combination thereof, are used to co-transfect a host cell, wherein each DNA fragment comprises a partial sequence of the MVA genomic DNA with overlapping sequences at the ends of two adjacent DNA fragments, such that when the two or more DNA fragments are co-transfected into the host cell, they assemble with each other by homologous recombination to form MVA comprising a full-length sequence of the desired MVA genome. In certain embodiments, the overlapping sequence is between about 100 bp and about 5000 bp in length.

In certain embodiments, one or more naturally derived or chemically synthesized DNA fragment(s) comprising the MVA genome or subgenomic DNA may be further modified to form artificial hybrid fragments composed of natural and synthetic MVA genomic DNA sequences.

In certain embodiments, the host cell is infected with a helper virus such as FPV before, during, or after the transfection of one or more DNA fragments comprising the sequence of the MVA genome or subgenomic DNA.

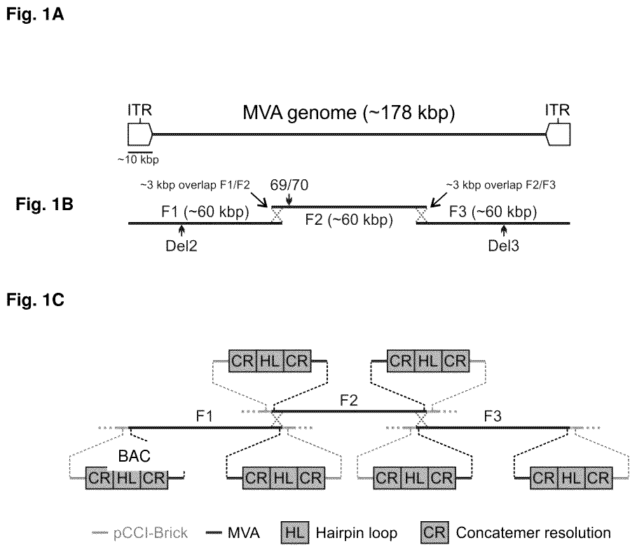

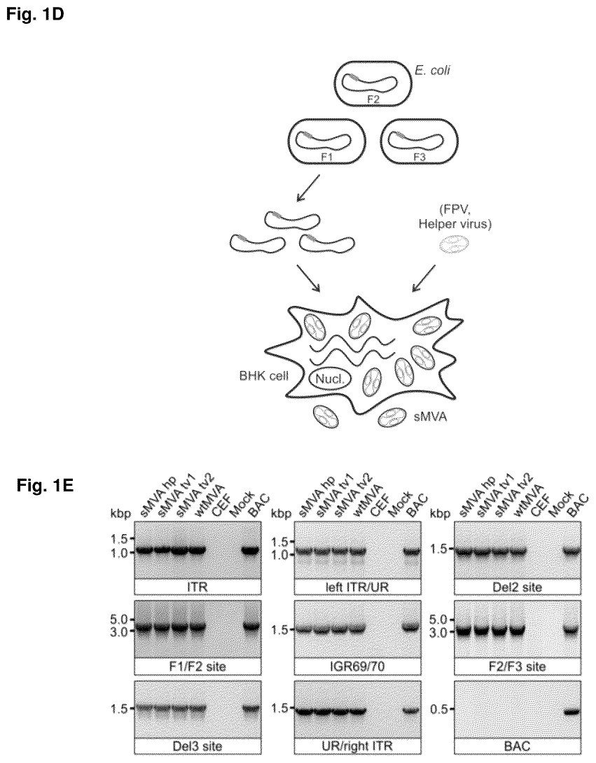

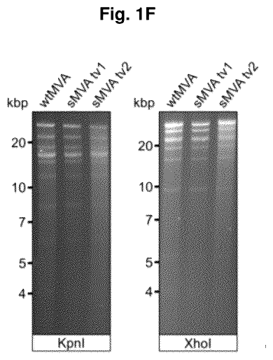

As demonstrated herein, the disclosed technique of generating sMVA involves the use of three large circular DNA fragments (about 60 kbp) with intrinsic HL and CR sequences (FIG. 1), the approach by Noyce et al. to produce a synthetic horsepox vaccine involves the use of multiple smaller linear DNA fragments (about 10-30 kbp) and the addition of terminal HL sequences⁴². Because the three sMVA fragments are used in a circular form for the sMVA reconstitution process they are easily maintained in E. coli as BACs and transferred to BHK-21 cells for sMVA virus reconstitution without the need for additional purification steps or modifications. This feature greatly facilitates the insertion of heterologous antigen sequences into the sMVA DNA by highly efficient bacterial recombination techniques and to produce recombinant sMVA vaccine vectors. Additionally, the three plasmid system provides the flexibility for rapid production of recombinant MVA harboring multiple antigens inserted into different MVA insertion sites, which can be particularly laborious when generating recombinant MVA by the conventional transfection/infection procedure^{3,43}. The three sMVA fragments efficiently recombine with one another and produce a synthetic form of MVA that is virtually identical to wtMVA in genome content, replication properties, host cell range, and immunogenicity.

More specifically, as illustrated in FIG. 1A, the MVA genome comprises an internal unique region (UR) flanked by ˜9.6 kbp long inverted terminal repeat (ITR) regions. The MVA genome sequence published by Antoine and colleagues (Accession #U94848), herein referred to as MVA strain Antoine, differs in five base pairs in the internal UR from the MVA genome of the licensed and commercially available National Institute of Health clone 1 from 1974 (MVA NIH clone 1), which is identical in sequence to the published genome of MVA strain Acambis (Accession #AY603355). sMVA fragment 1 (F1) encompasses the left ITR and ˜50 kbp of the left end of internal UR of the MVA genome; sMVA fragment 2 (F2) contains ˜60 kbp of the middle part of the internal UR of the MVA genome; and sMVA fragment 3 (F3) encompasses ˜50 kbp of the right end of the internal UR and the right ITR of the MVA genome (FIG. 1B). sMVA F1 and F2 as well as sMVA F2 and F3 are designed to share ˜3 kbp overlapping sequences to allow the reconstitution of the complete MVA genome by homologous recombination (FIG. 1B). A duplex copy of the 165-nucleotide long MVA terminal hairpin loop (HL) flanked by MVA concatemeric resolution sequences is added to both ends of each of the three fragments to promote MVA genome resolution and packaging (FIG. 1C). The three sMVA fragments are cloned and maintained in E. coli (DH10B, EPI300, GS1783) by a yeast-bacterial shuttle vector, termed pCCI-Brick (GeneScript), which contains a bacterial mini-F replicon element that can be used as a BAC vector to stably propagate the three fragments at low copy number in bacteria (FIG. 1). Next generation sequencing analysis confirmed the integrity of the MVA genomic sequences of the fragments, with the notable exception of an unknown single point mutation within sMVA fragment F1 that is located in a non-coding determining region at 3 bp downstream of 021L.

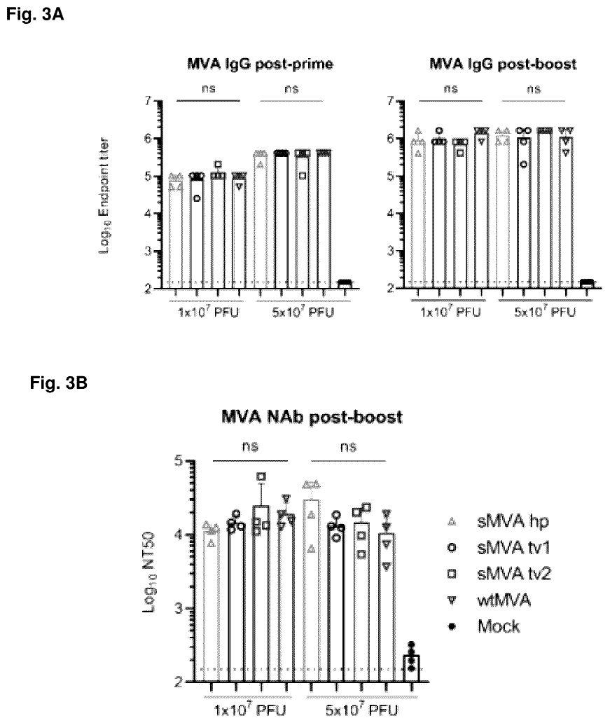

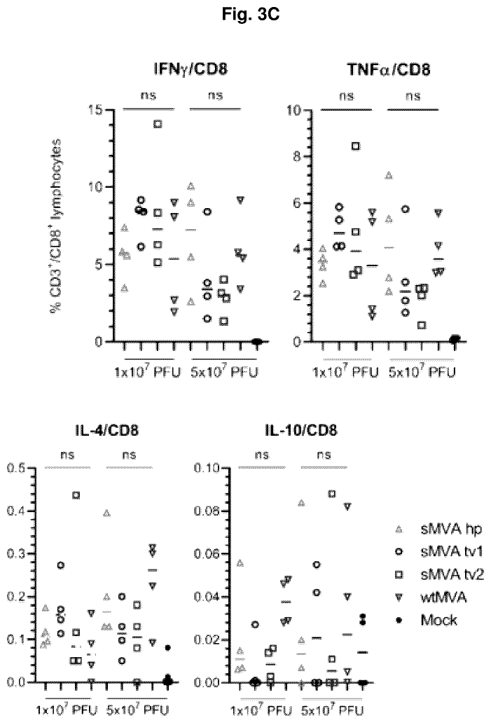

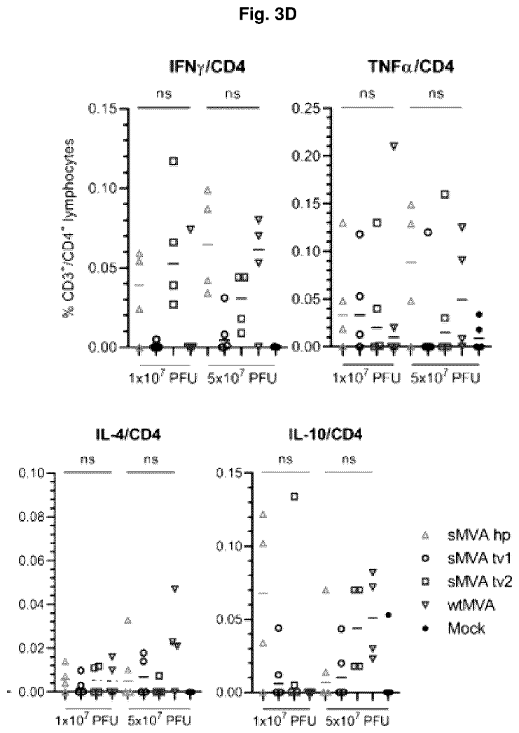

When baby hamster kidney (BHK) are co-transfected with the three plasmids containing the sMVA fragments F1-F3 (FIG. 1) and subsequently infected with Fowlpox virus (FPV) as a helper virus, the three sMVA fragments recombine with each other through the shared homologous sequences and the reconstitution of synthetic MVA (sMVA) is initiated (FIG. 3D). FPV is used as a helper virus to initiate the transcription of the sMVA DNA and, consequently, the sMVA reconstitution process. In the absence of a helper virus, the “naked” sMVA DNA may not promote virus reconstitution as poxvirus DNA is considered as non-infectious. Importantly, while BHK-21 cells are highly permissive for MVA infection and replication, they are not permissive for productive FPV infection, leading to immediate removal of the FPV helper virus following sMVA virus reconstitution in BHK-21 cells. In addition, FPV used as a helper virus in mammalian cells promotes highly efficient and selective packaging of vaccinia virus genomes.

Also disclosed is a multi-antigenic sMVA-CoV2 vaccine using the highly versatile synthetic vaccine platform based on sMVA. MVA is a highly attenuated poxvirus vector, widely used to develop vaccines for infectious diseases and cancer. There is a long history of safety, efficacy and long-term protection in humans. New Spike variants of SARS-CoV-2 can be quickly cloned into one of three plasmids that when recombined form an sMVA vaccine.

As disclosed herein, the multiple antigens, subunits thereof, or fragments thereof can be co-expressed using the same promoter or different promoters, optionally linked by 2A peptides. The sequences encoding the multiple antigens, subunits thereof, or fragments thereof can be inserted at the same insertion site or different insertion sites of sMVA. For example, the vaccine composition comprising two or more antigens encoding for at least two S or N proteins, S1 or S2 domains, or RBDs are co-expressed using the same promotor or separate promoters or the same insertion site or separate insertion sites. In another example, the vaccine composition comprising two or more antigens encoding for at least two S or N proteins, S1 or S2 domains, or RBD are linked by 2A peptides and co-expressed through polycistronic constructs by the same promoter.

In certain embodiments, the vaccine composition disclosed herein comprises a mixture of two or more sMVA vectors which encode two or more different SARS-CoV-2 antigen sequences selected from the Wuhan-Hu-1 reference strain and different VOC. For example, one sMVA vector in the mixture comprises sequences encoding SARS-CoV-2 antigens from the Wuhan-Hu-1 reference strain, and another sMVA vector in the mixture comprises sequences encoding SARS-CoV-2 antigens from a VOC.

MVA is a highly attenuated strain of vaccinia. Mammalian cells are permissive to MVA, including human cells, propagation restricted to avian cells. MVA also has multi-antigenic capacity (30 Kb) and can be easily modified to assemble new vaccines against viral variants, e.g. UK or RSA variants. MVA is an attenuated viral vaccine, which has advantage in immunogenicity, compared to DNA/RNA/protein vaccines. MVA is capable of long-lived high titer humoral and high frequency cellular immune responses, maintains immunogenicity as lyophilizate to eliminate cold chain resulting in cheaper storage and transport. Safety and efficacy of MVA-based vaccines were established in human trials since the 1970s. Over 150,000 people were successfully immunized in historical studies, including children and the elderly. Multiple studies sponsored by NIAID showed safety after immunization in HIV-infected adults. MVA is suitable for providing lifelong immunity against smallpox based on FDA approval as Jynneos™ (Bavarian-Nordic). Multiple MVA-based vaccines have been developed and successfully investigated at COH. Healthy volunteers and transplant patients develop strong immunity even after a single dose.

In contrast to most other currently employed SARS-CoV-2 vaccine approaches that solely rely on the S antigen, the disclosed SARS-CoV-2 vaccine approach using sMVA employs immune stimulation by S and N antigens, both are implicated in protective immunity^{20,26}. The observation that the sMVA-CoV2 vectors co-expressing S and N antigens can stimulate potent NAb against SARS-CoV-2 pseudovirus and infectious authentic SARS-CoV2 virions suggests that they can elicit antibodies that are considered effective in preventing SARS-CoV-2 infection and COVID-19 disease^{16,18,20,21}. The working examples demonstrate that the vaccine vectors stimulated a Th1-biased antibody and cellular immune response, which is considered the preferred antiviral adaptive immune response to avoid vaccine associated enhanced respiratory disease^{44,45}. Moreover, no evidence is found for Fc-mediated ADE promoted by the vaccine-induced immune sera, suggesting that antibody responses induced by the vaccine vectors bear minimal risk for ADE-mediated immunopathology, a general concern in SARS-CoV-2 vaccine development^{44,45}. Other immune responses besides NAb targeting the S antigen may contribute to the protection against SARS-CoV-2 infection, which is highlighted by the finding that even patients without measurable NAb can recover from SARS-CoV-2 infection²⁰. While antibodies could be particular important to prevent initial SARS-CoV-2 acquisition, T cell responses may impose an additional countermeasure to control sporadic virus spread at local sites of viral infection, thereby limiting virus transmission. The disclosed dual recombinant vaccine approach based on sMVA to induce robust humoral and cellular immune responses to S and N antigens may provide protection against SARS-CoV-2 infection beyond other vaccine approaches using solely the S antigen.

sMVA recombinants are produced by inserting the sequences encoding one or more antigens or subunits thereof into one or more MVA fragments. In certain embodiments, the DNA sequences of the antigens, subunits, or fragments thereof are codon optimized for expression in the host cell. In certain embodiments, the one or more antigens include human coronavirus antigens such as the S protein, N protein, M protein, E protein, papain-like protease, ORF1A, 3CL protease, ORF1B, endoribonuclease, matrix, helicase, or immunogenic fragments thereof. In certain embodiments, the one or more antigens include a subunit of S protein such as S1 and S2 domains, or the receptor-binding domain (RBD) of the S antigen. In certain embodiments, the one or more antigens include a prefusion form of the S antigen and a mutated S antigen. For example, the SARS-CoV-2 S antigen can be further stabilized by including a mutated Furin cleavage site such that amino acid residues 682-685 RRAR are mutated to GSAS. In another example, lysine 986 and valine 987 of the S antigen are substituted with prolines. In certain embodiments, the S antigen and the N antigen are fully mature or fully glycosylated.

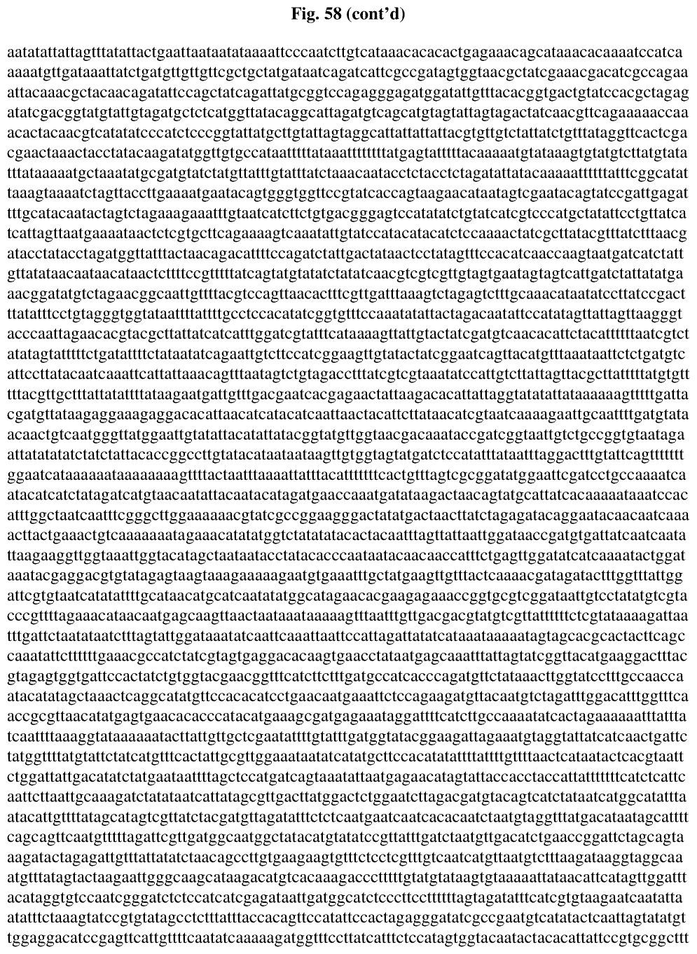

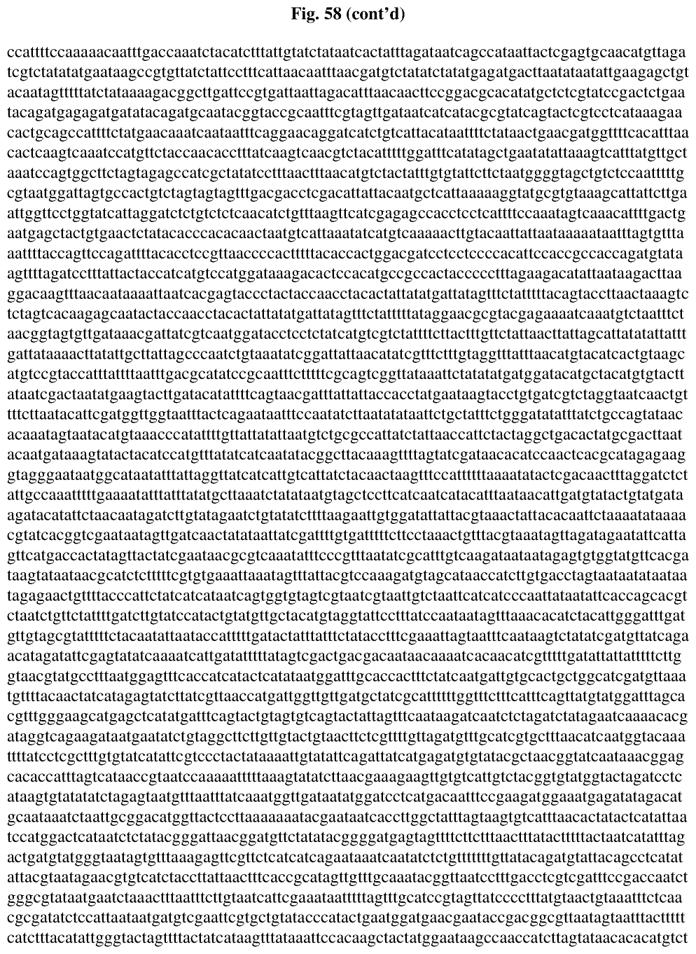

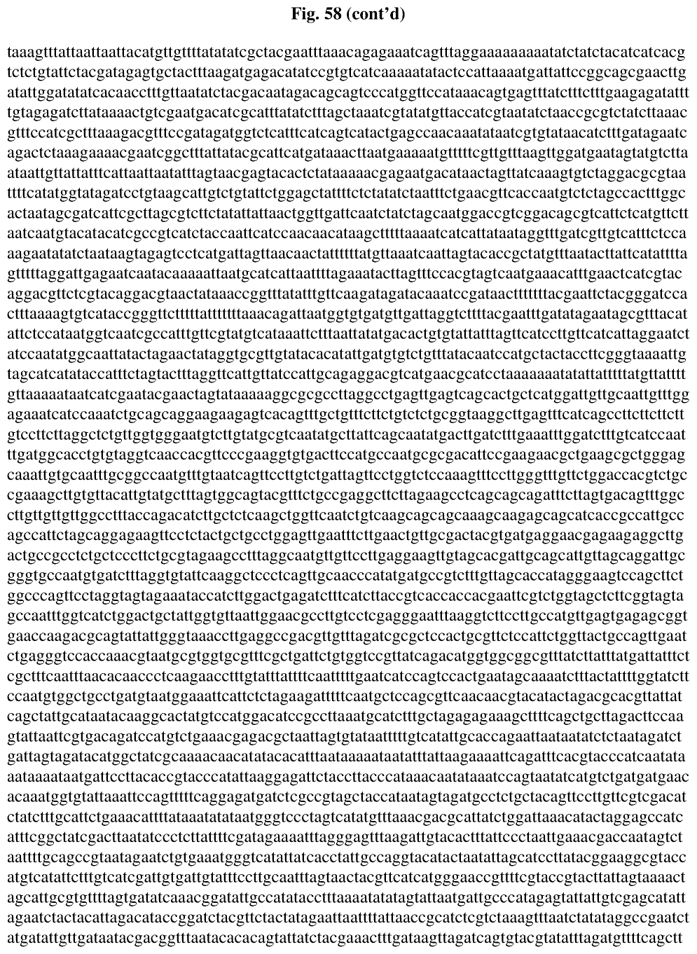

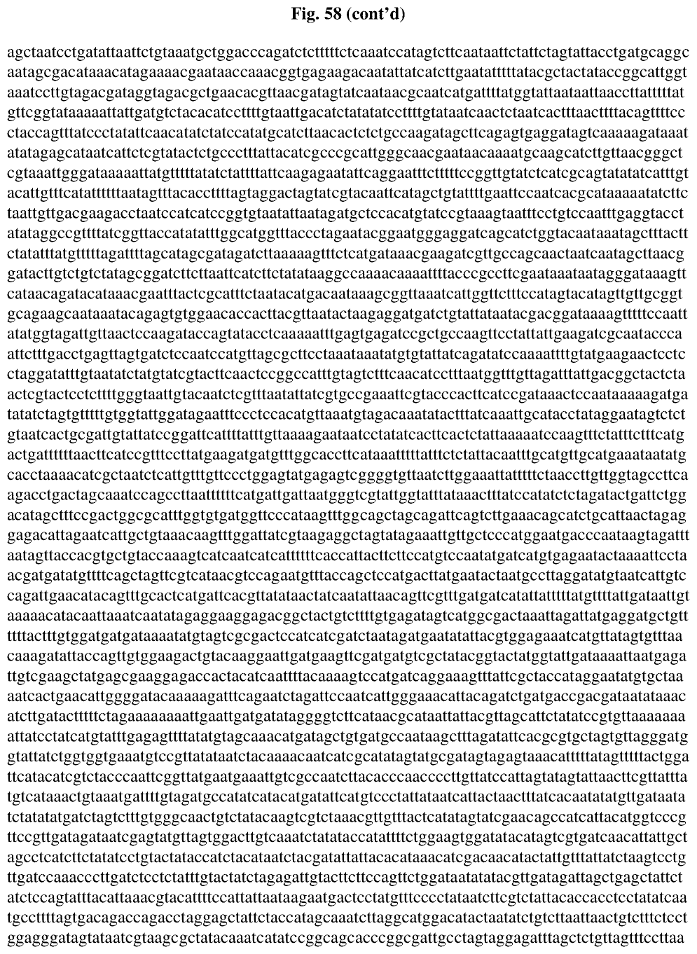

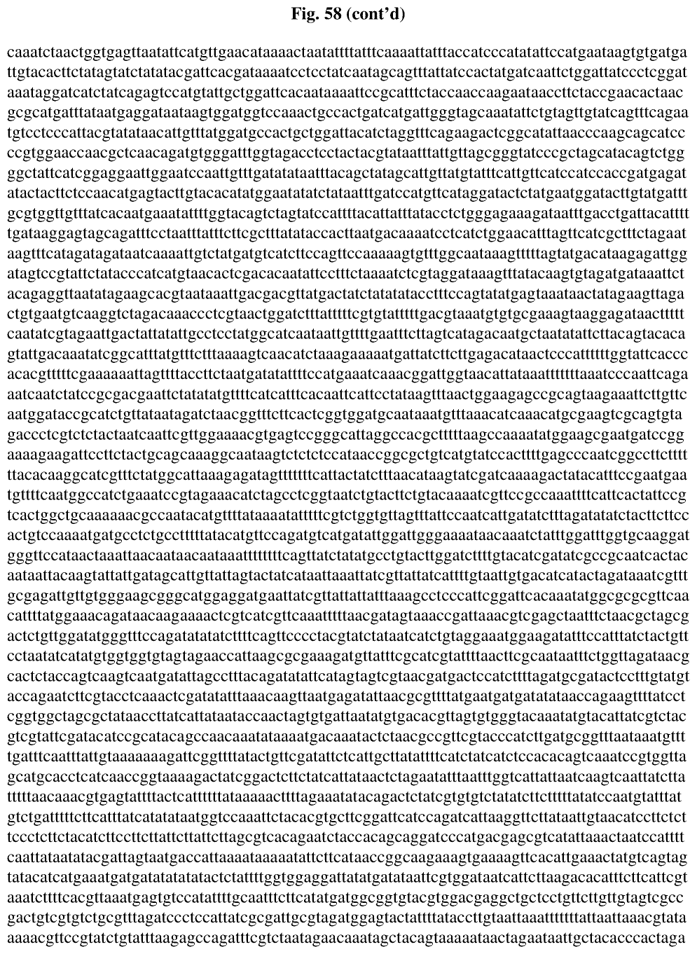

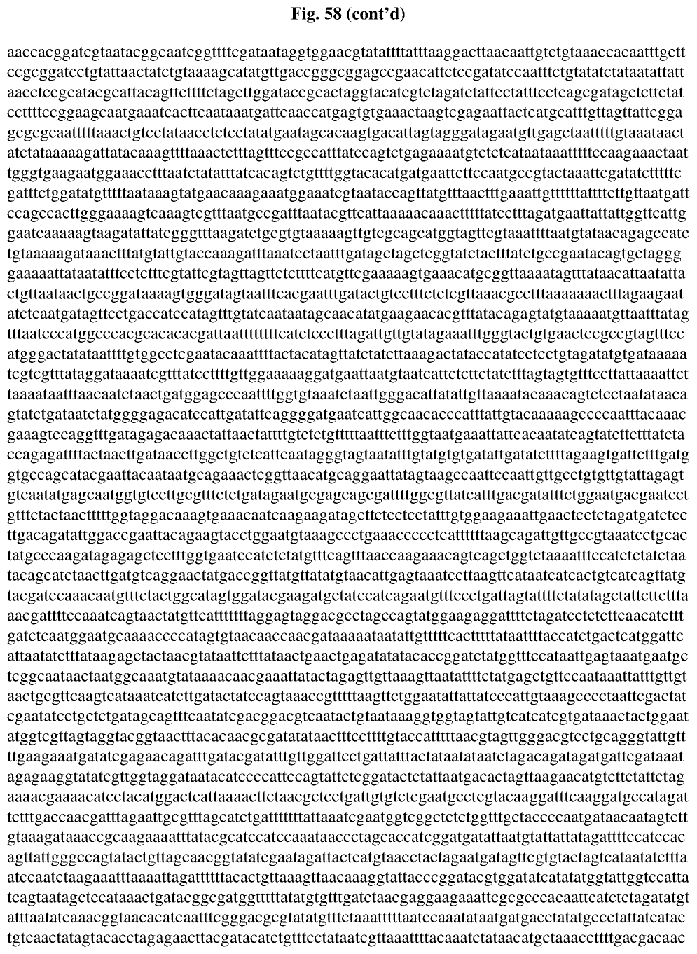

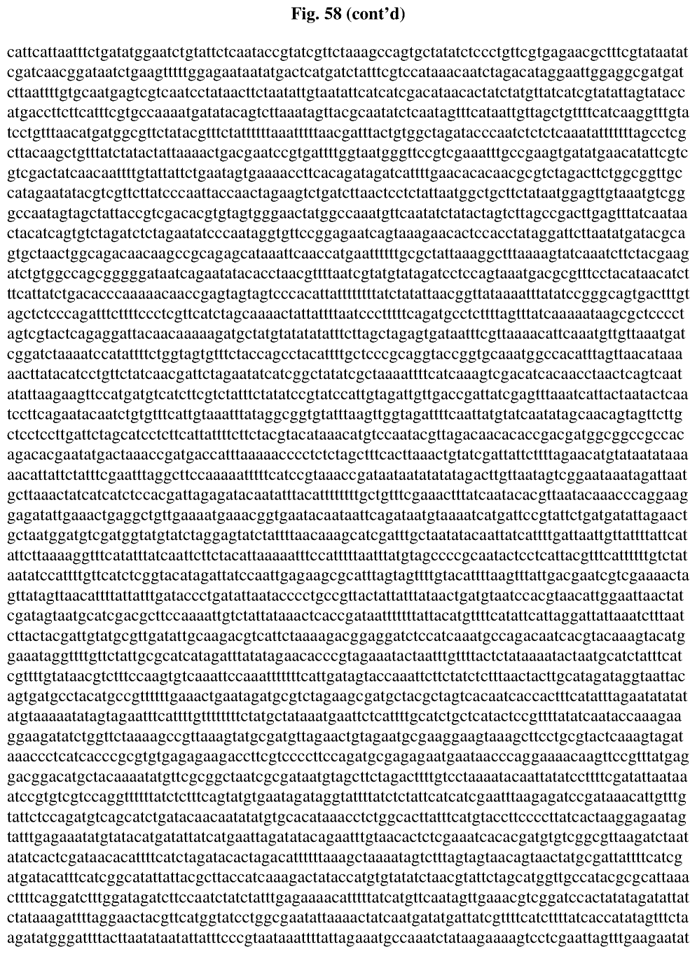

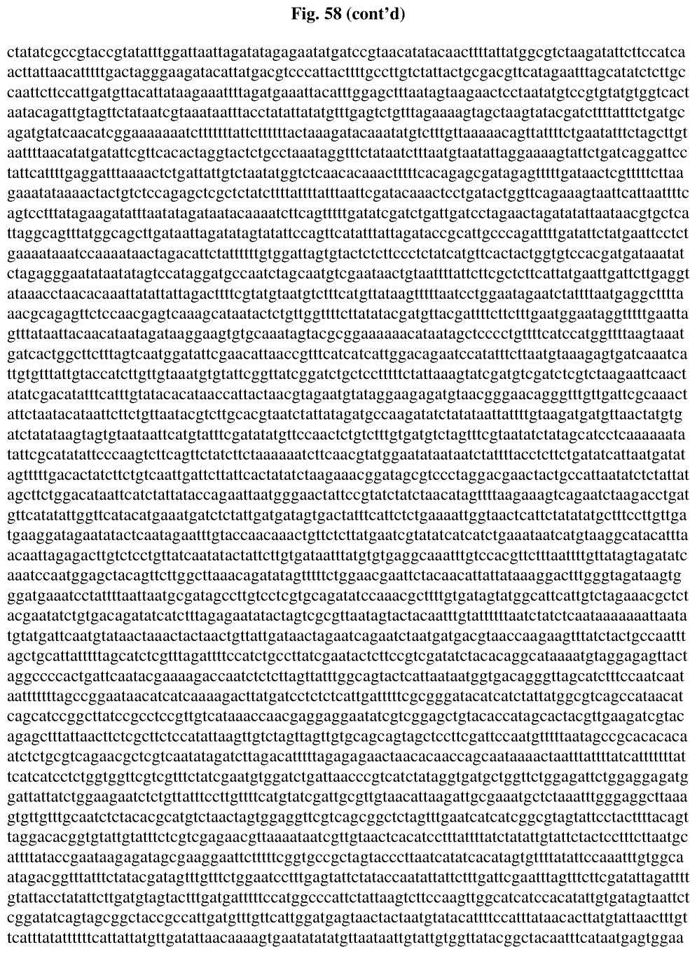

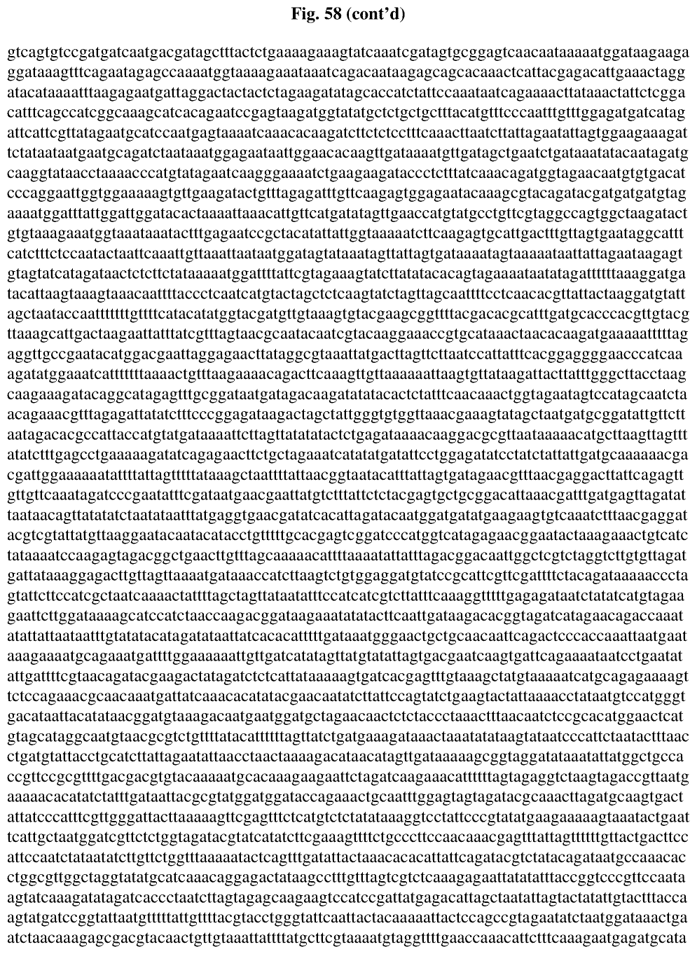

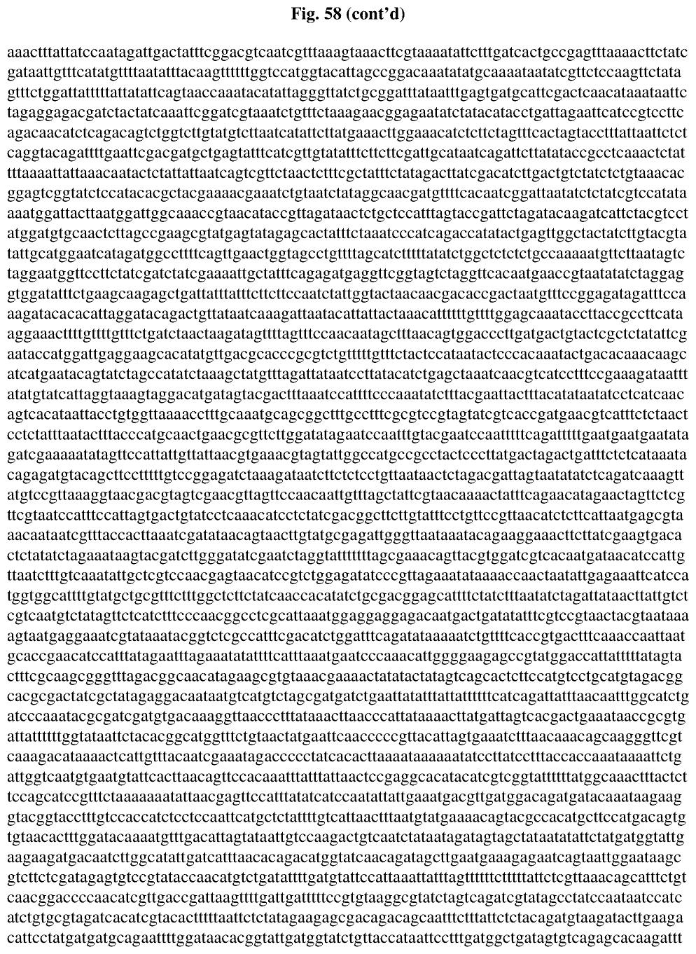

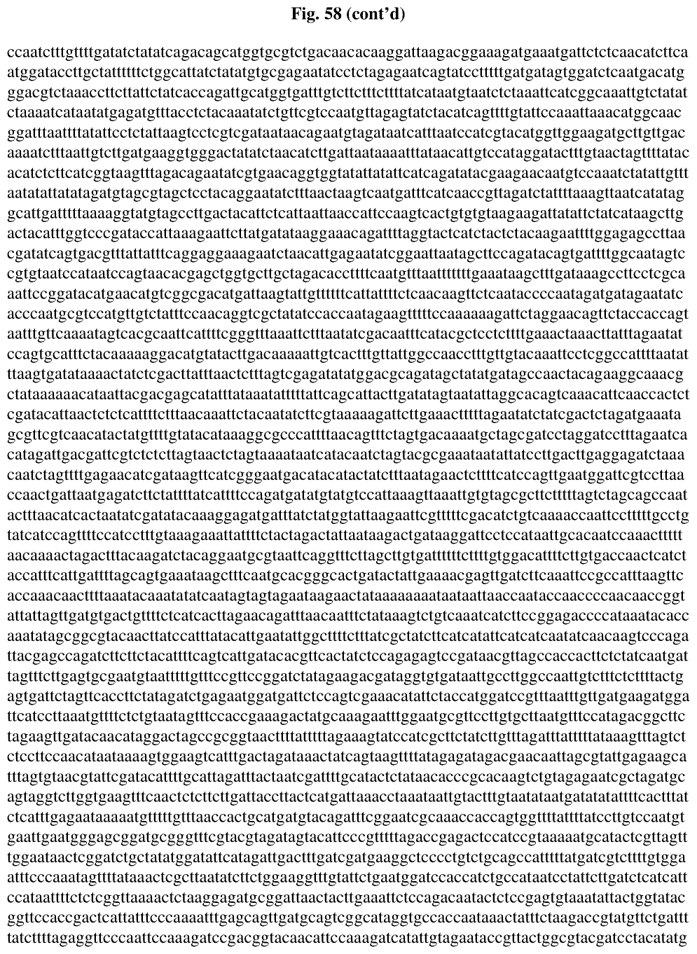

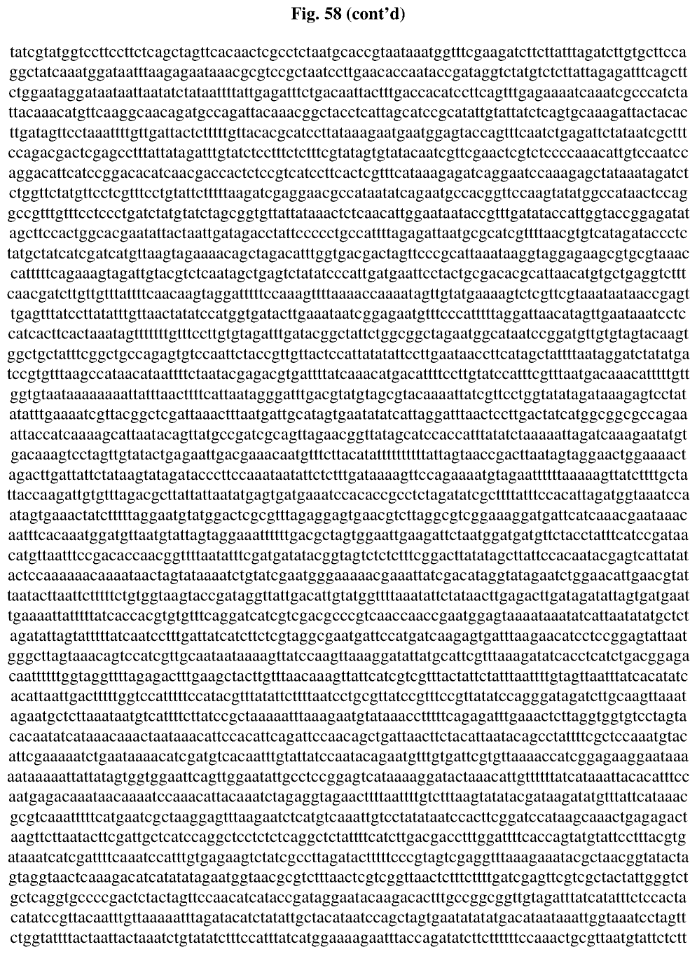

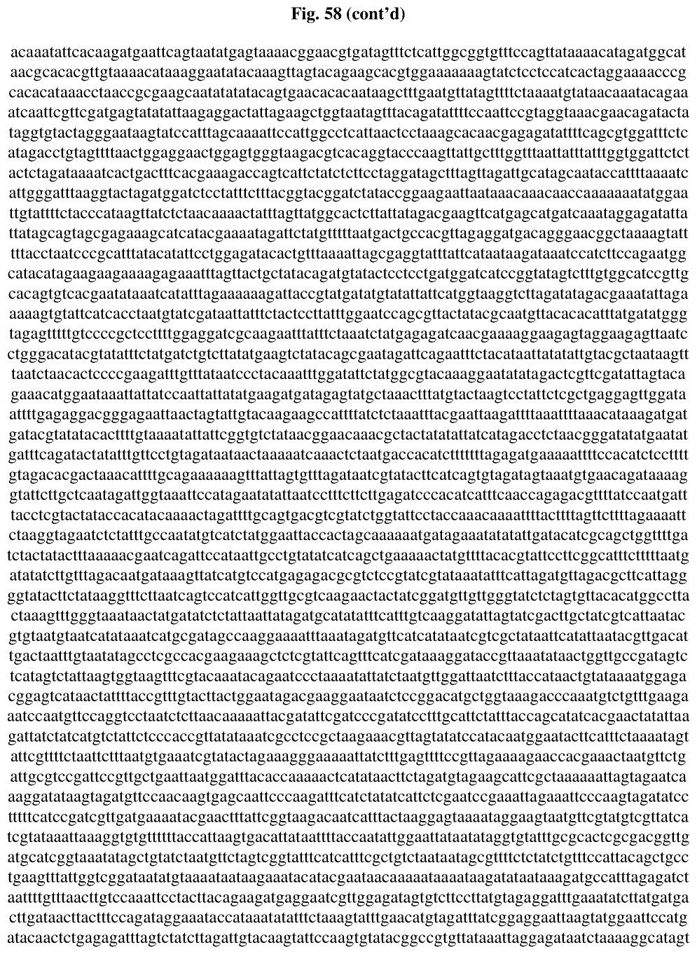

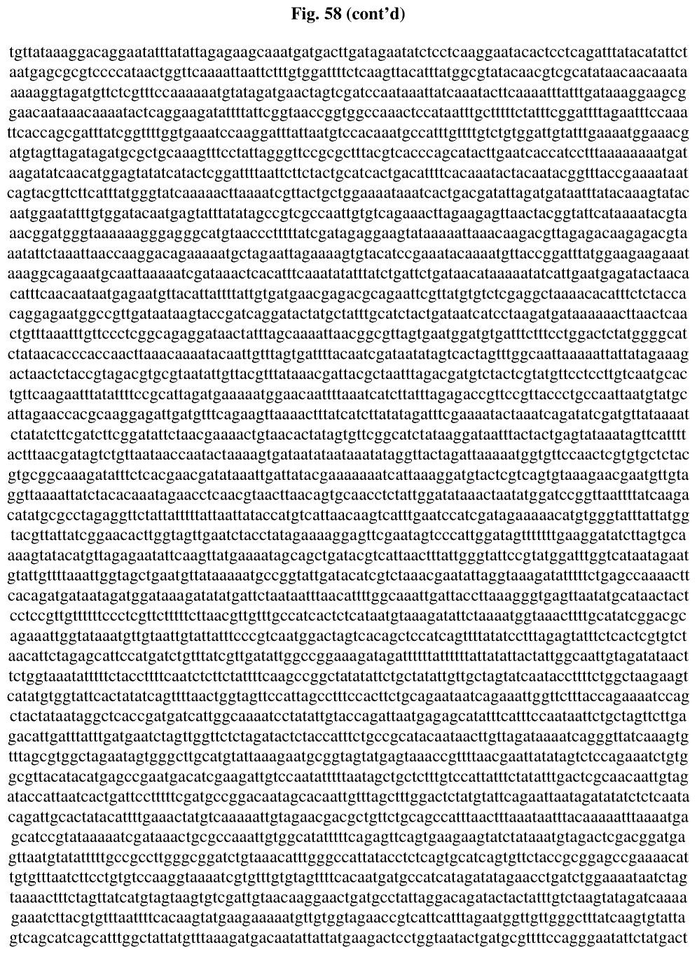

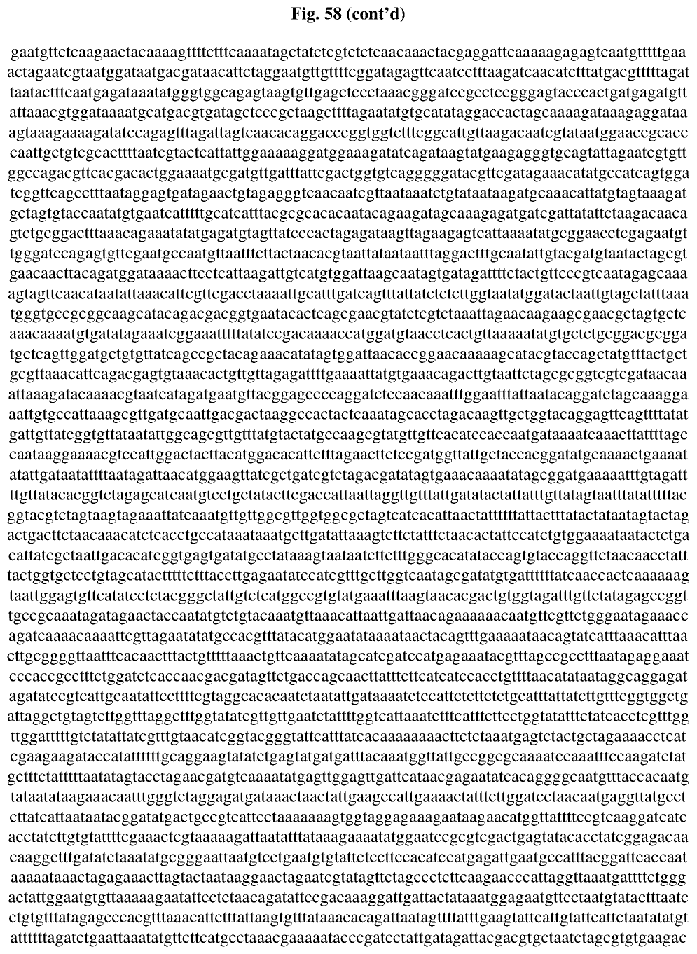

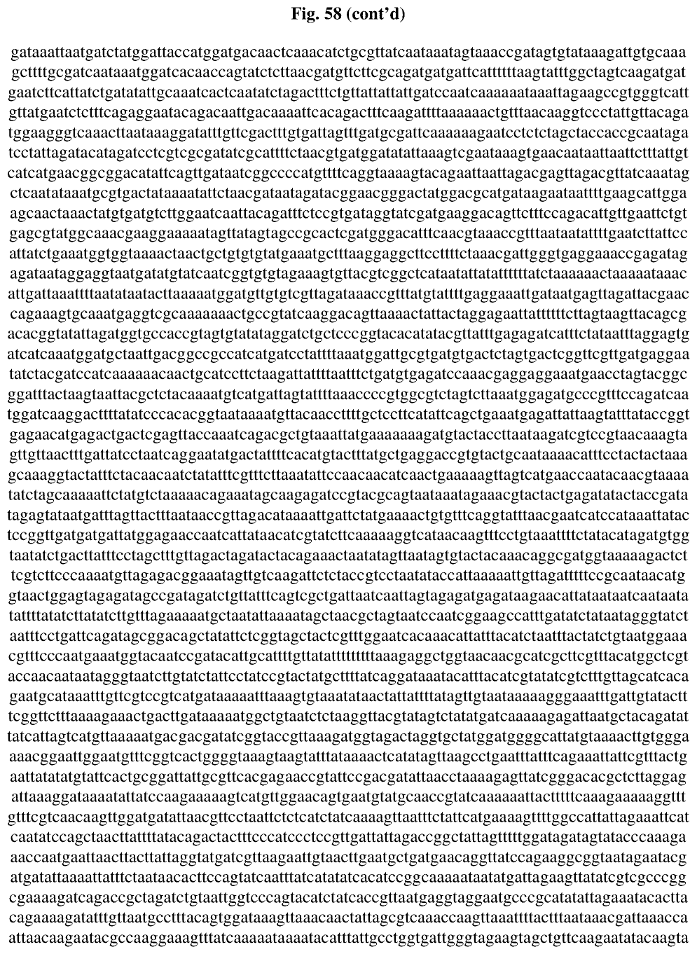

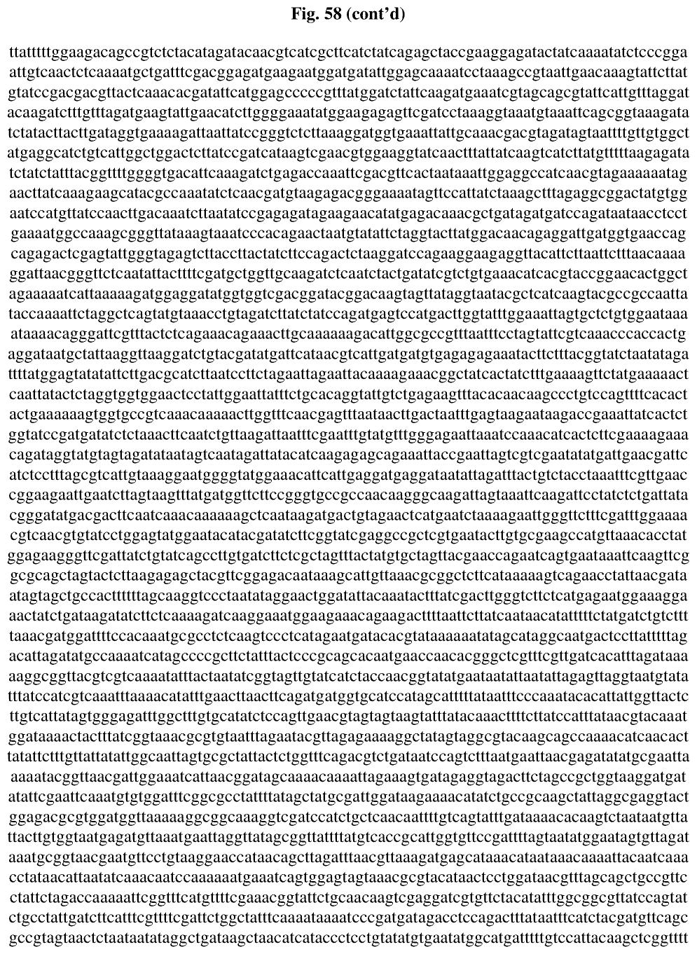

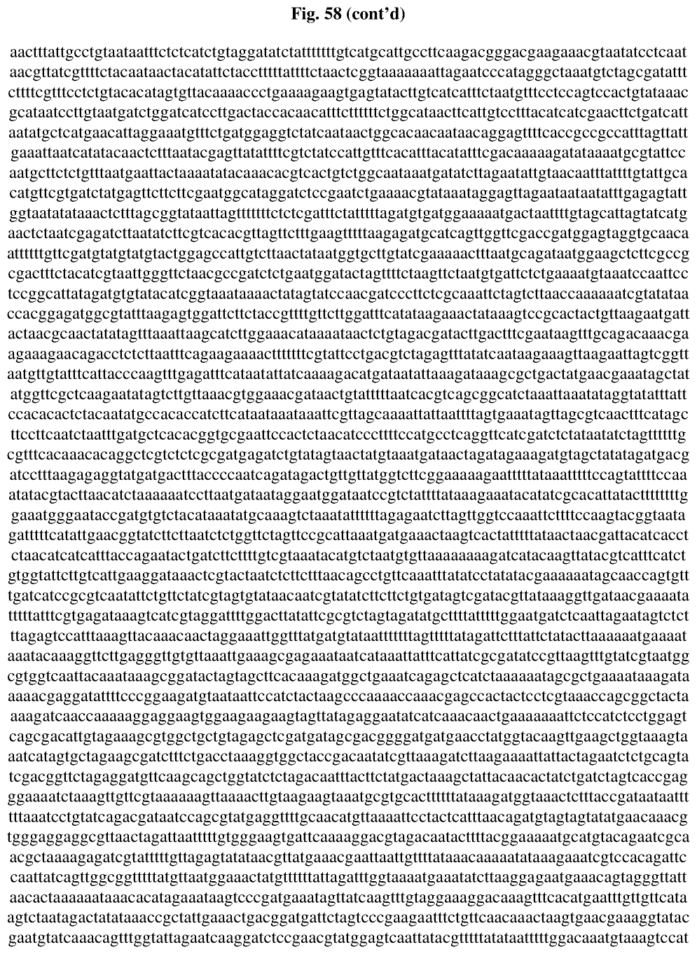

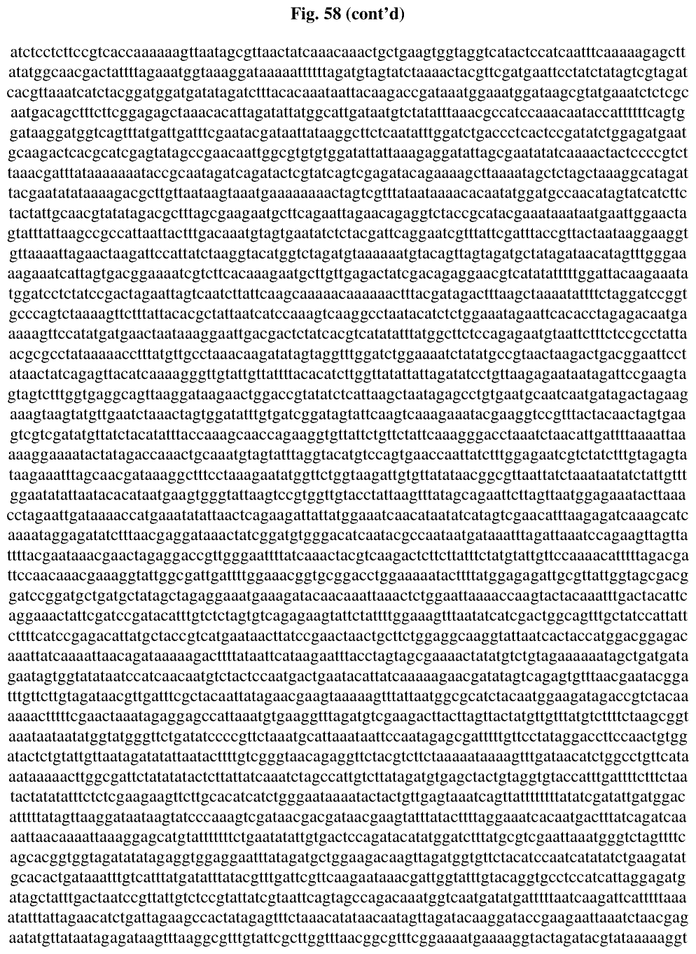

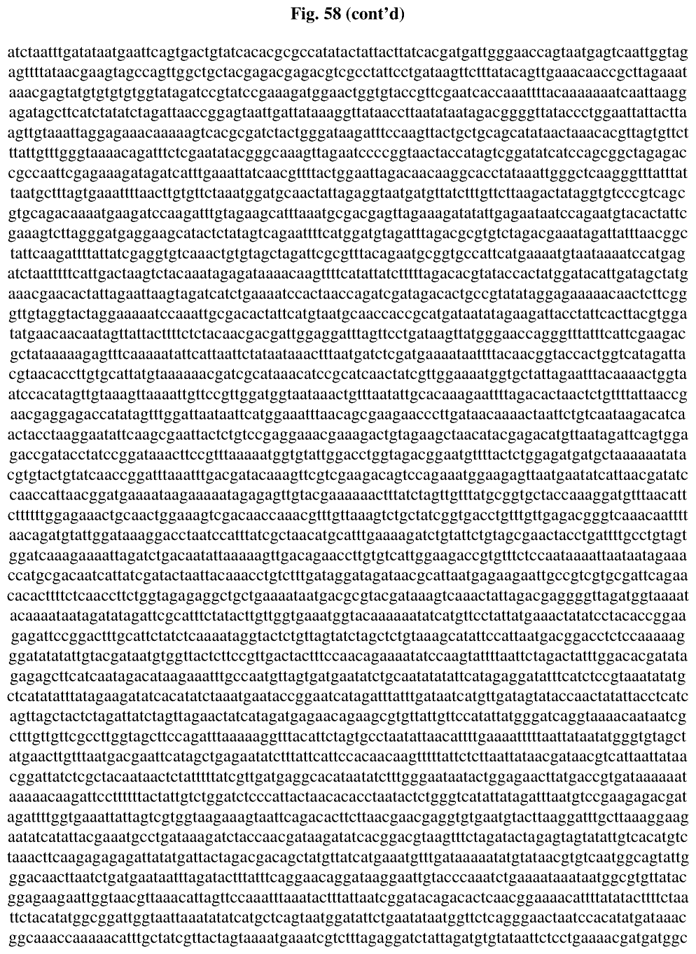

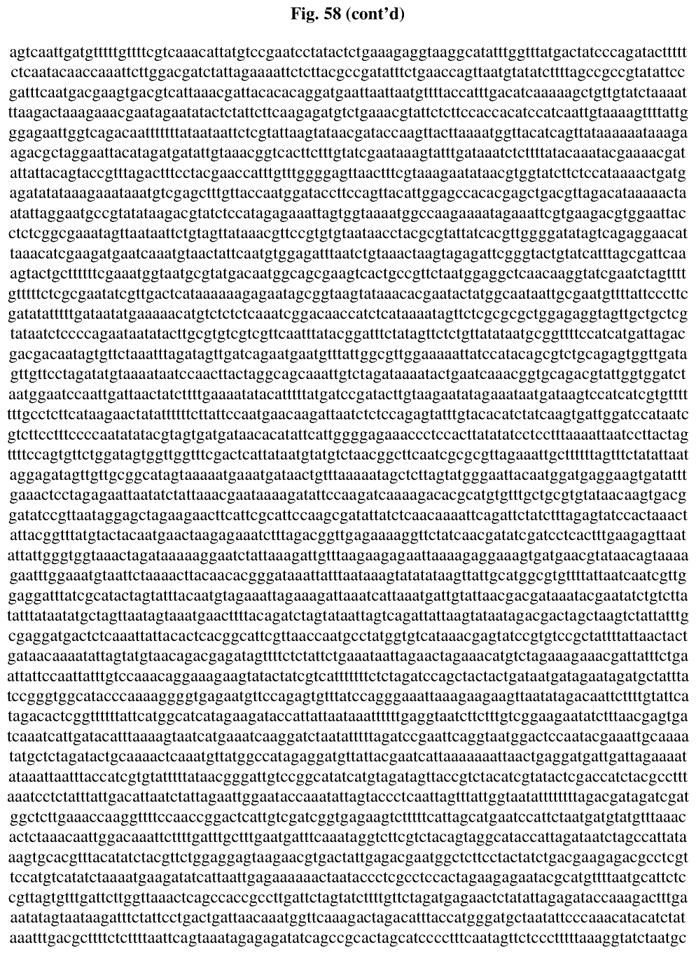

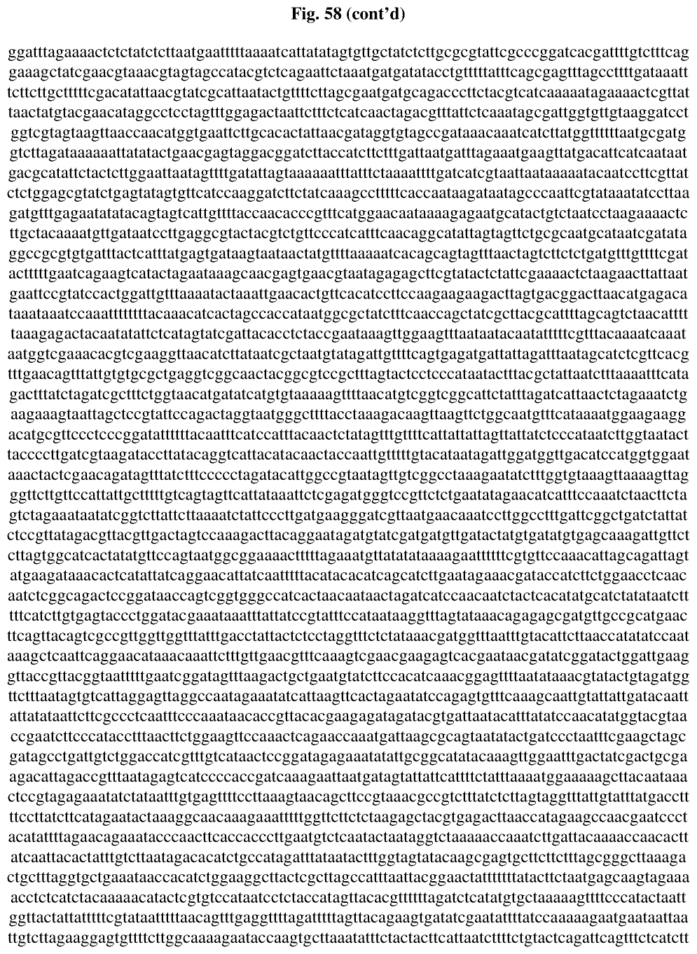

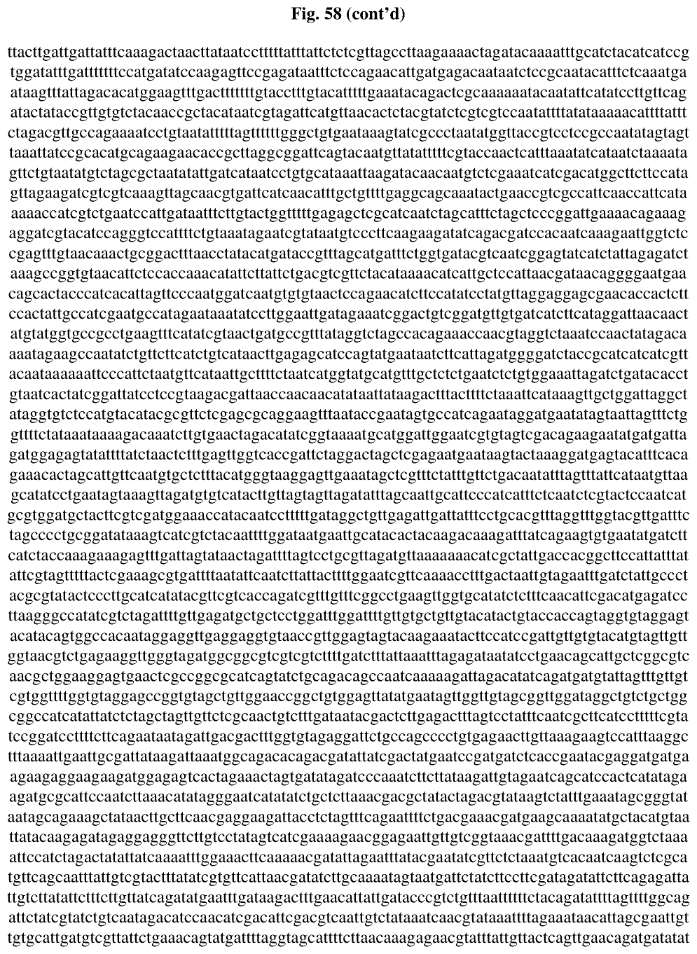

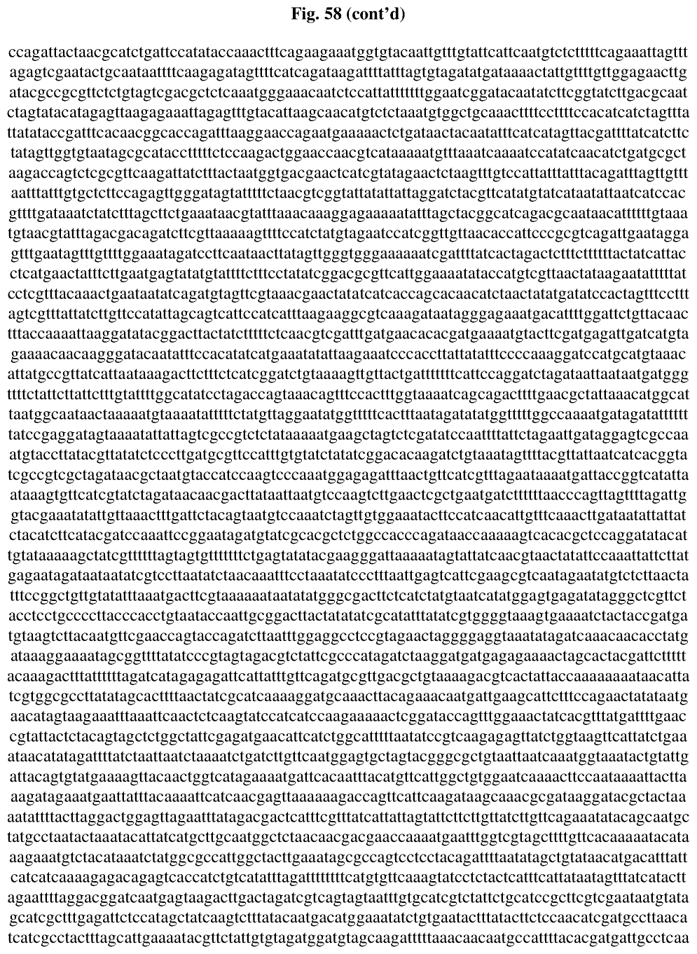

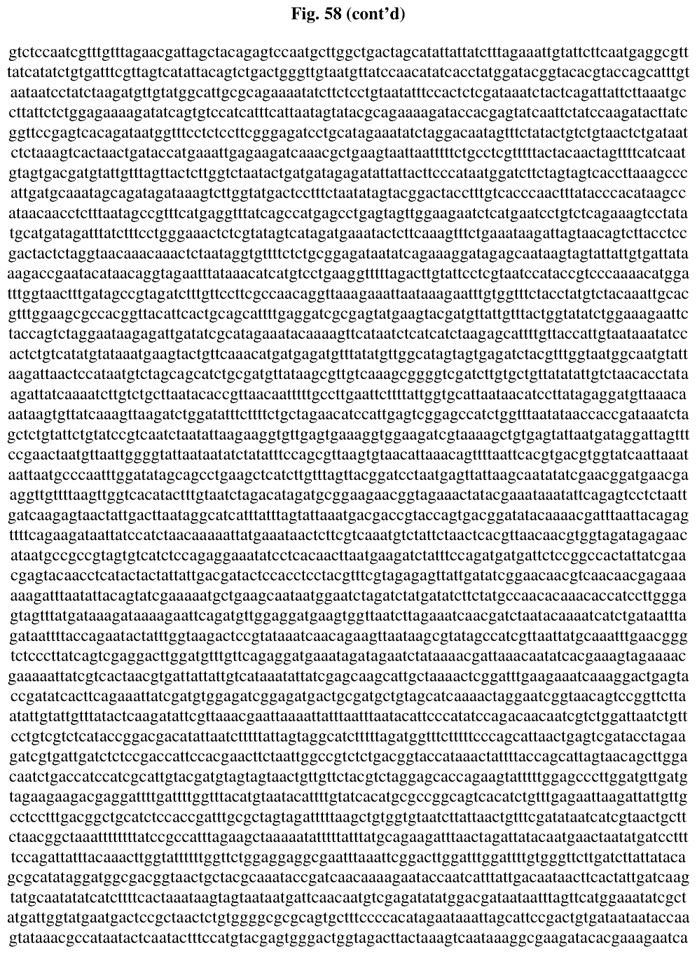

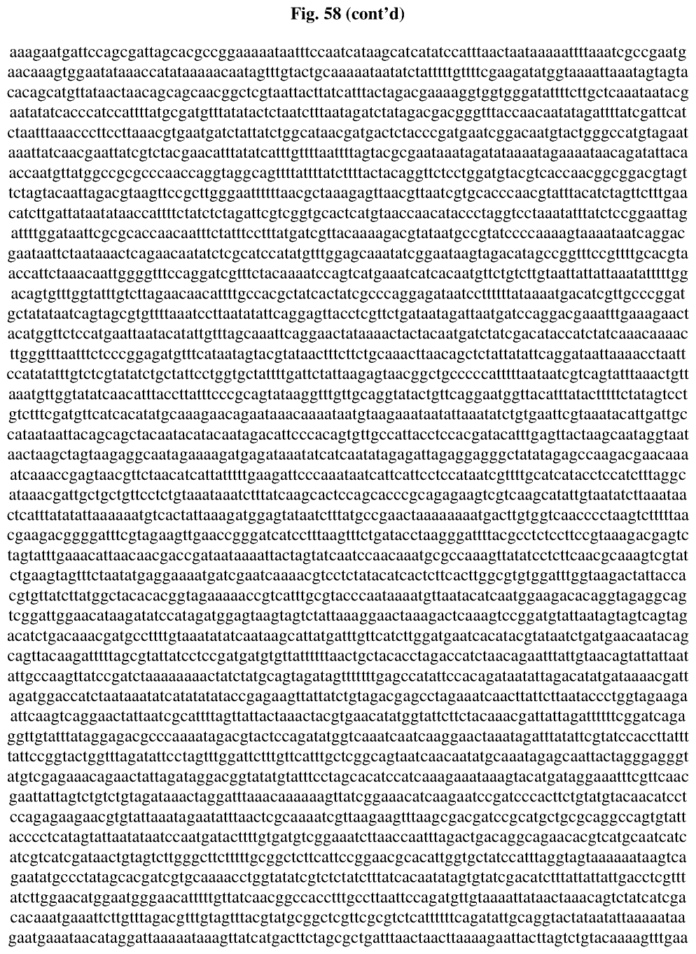

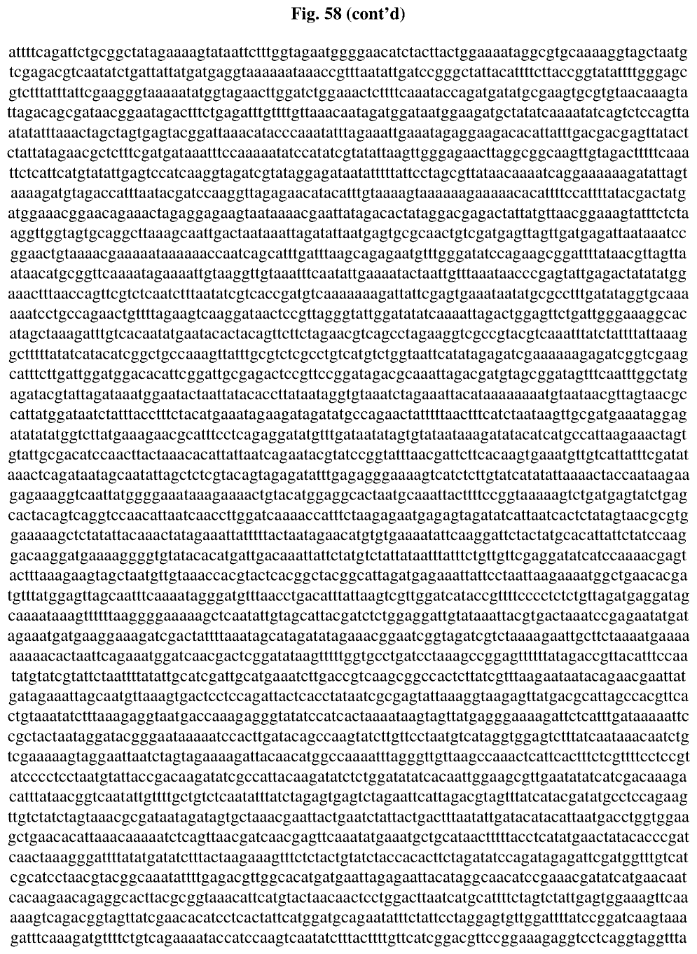

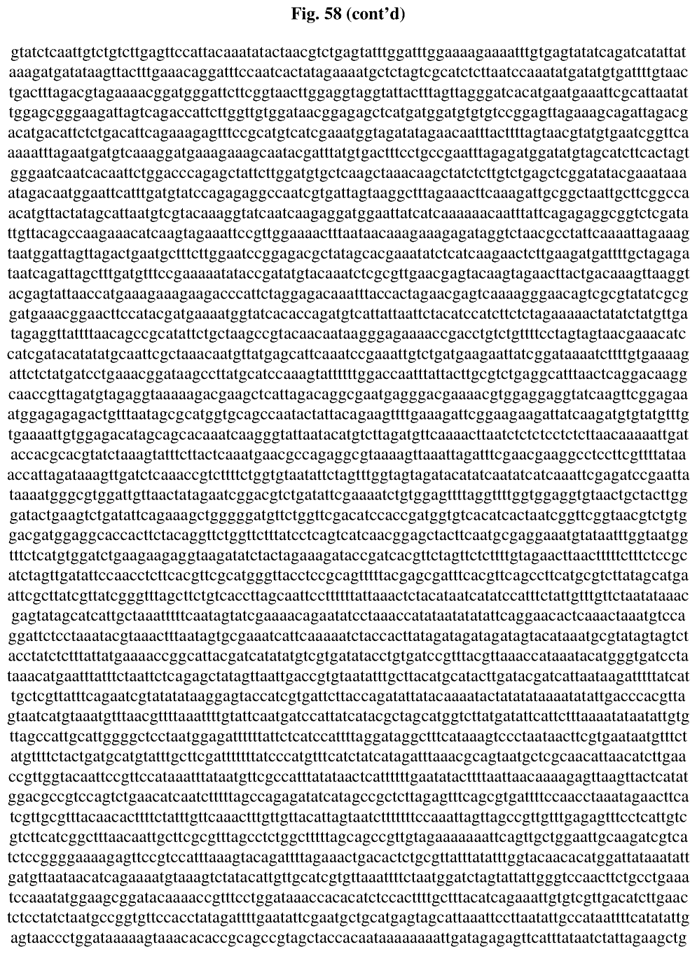

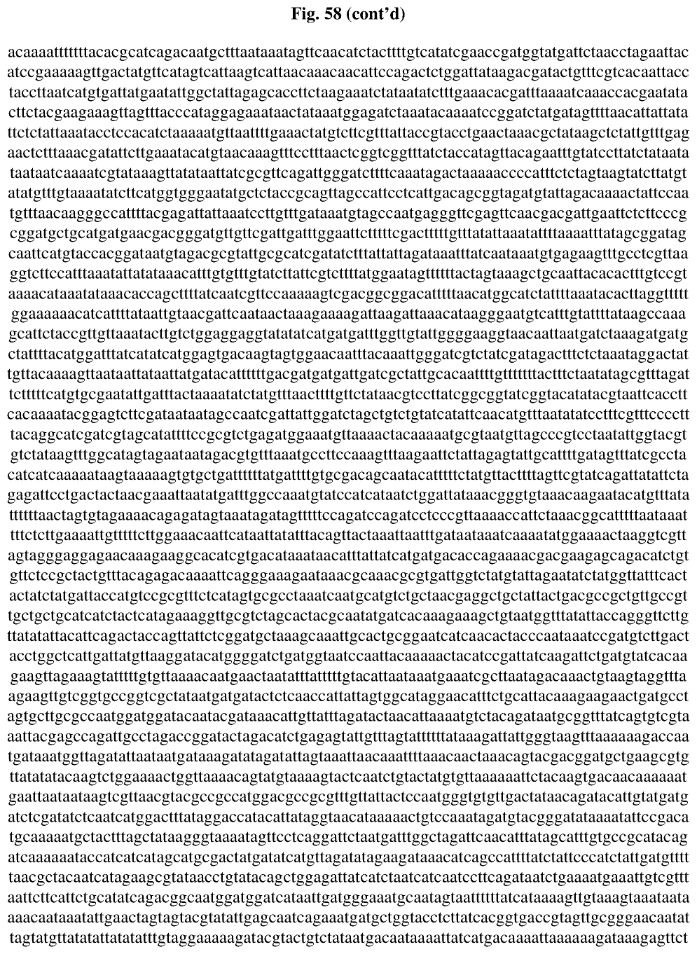

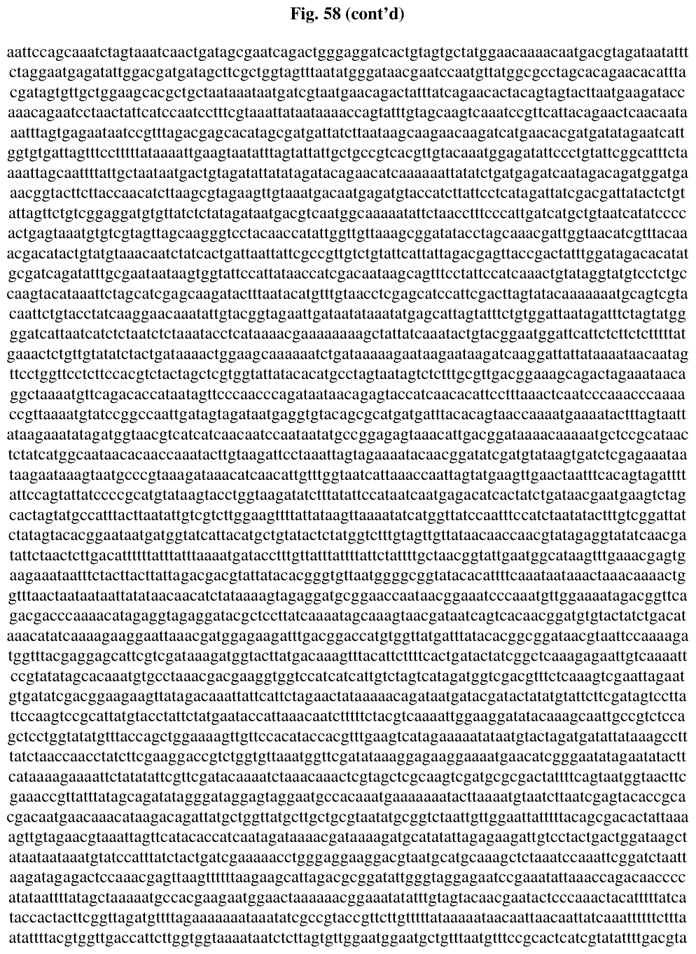

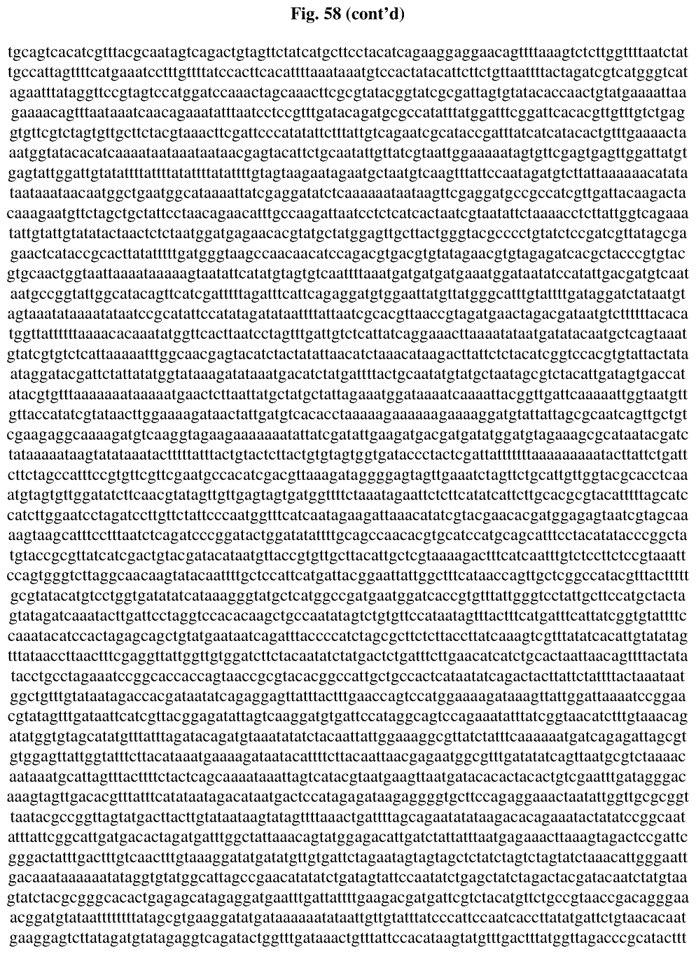

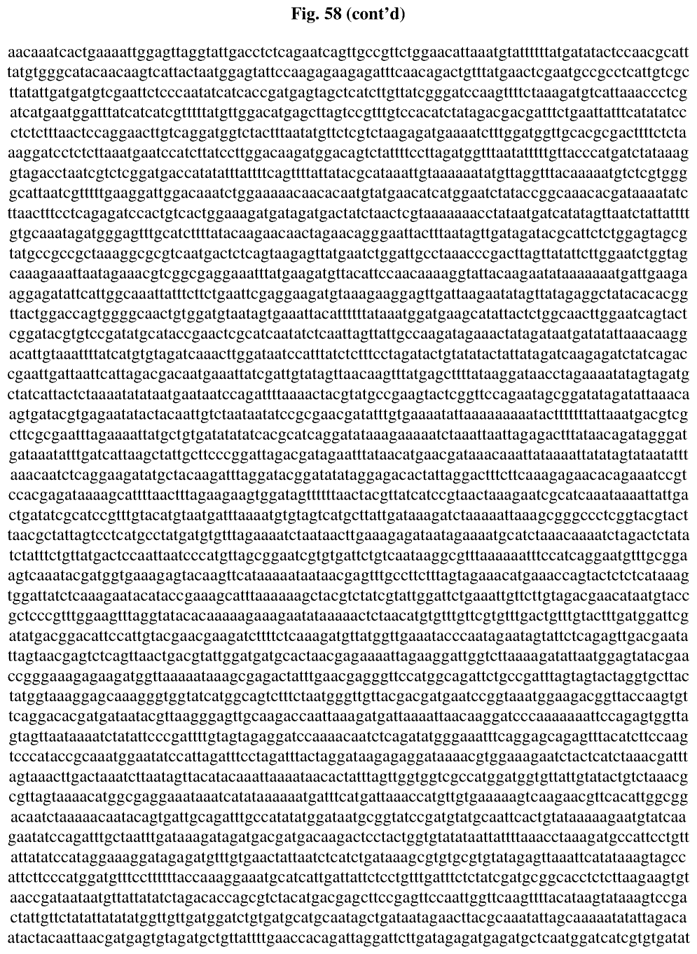

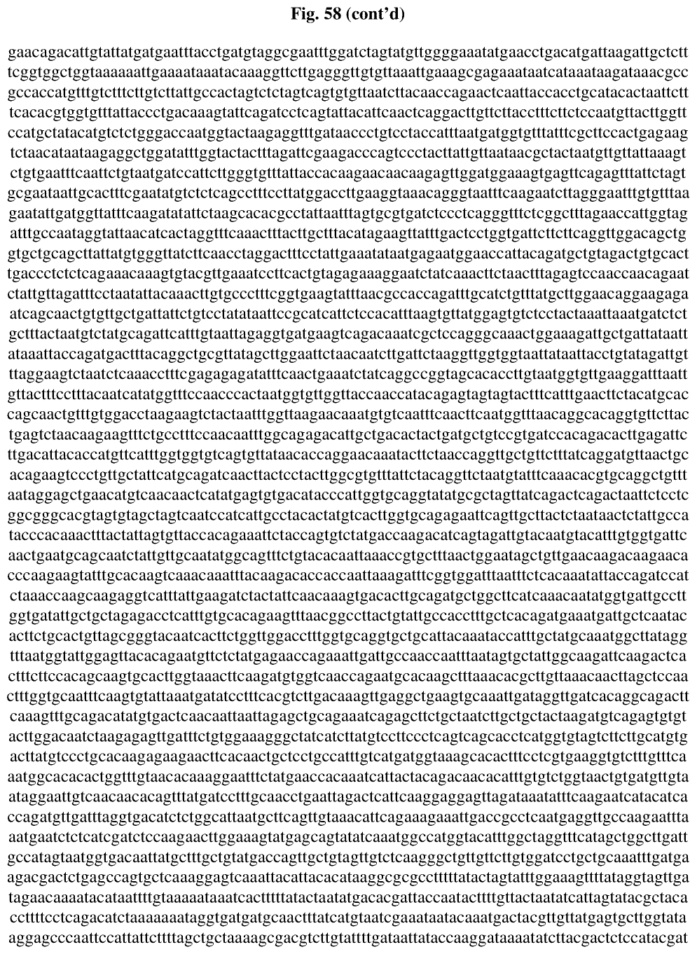

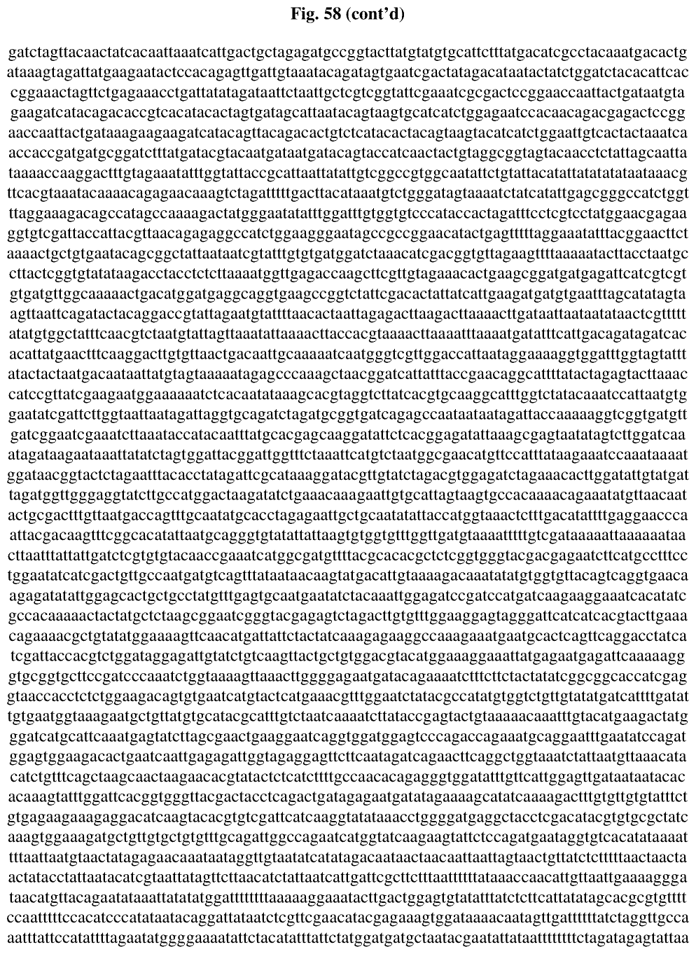

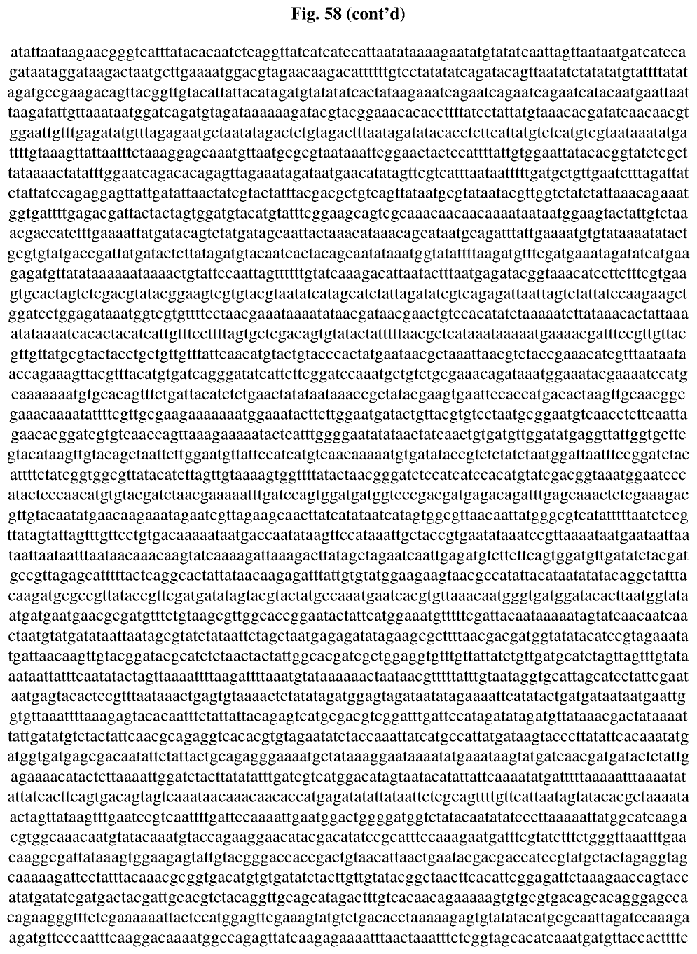

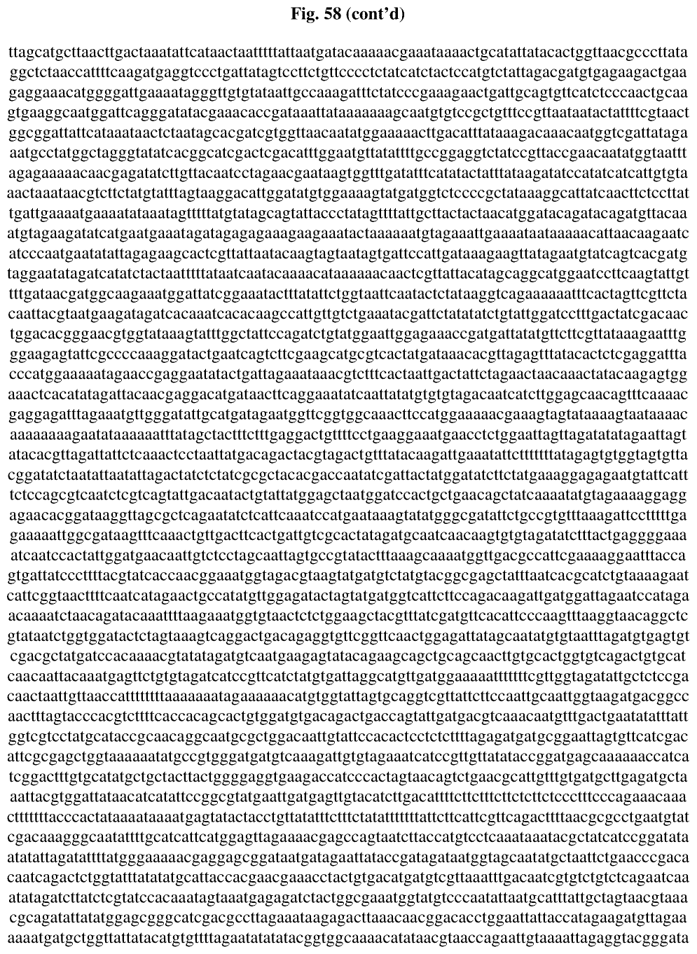

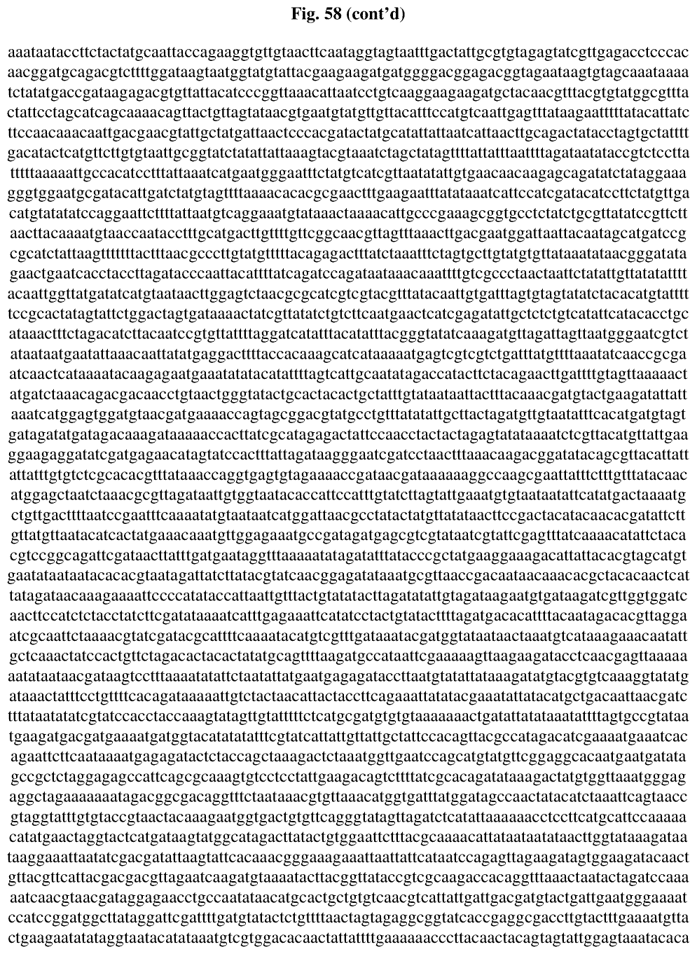

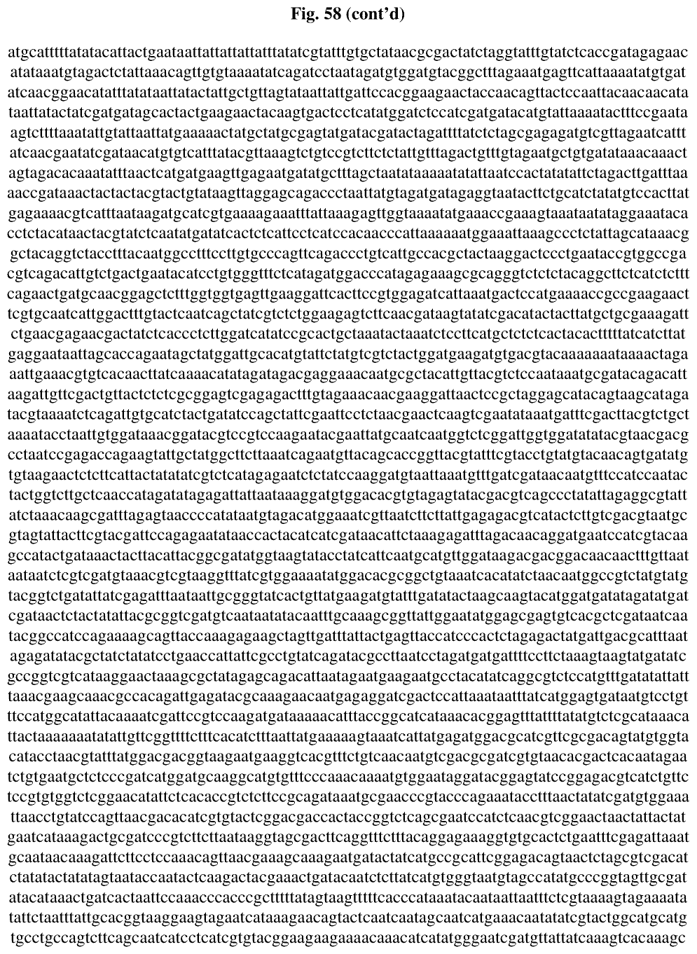

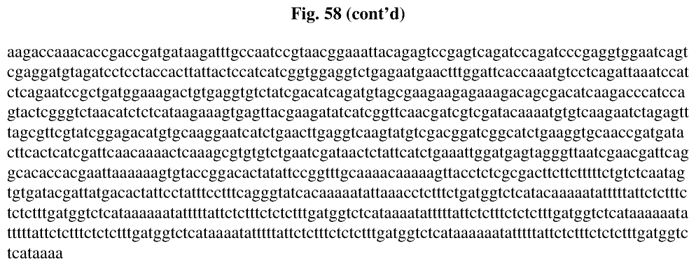

In certain embodiments, the sequence of sMVA-N/S (deposited with NCBI under Accession No. MW036243, www.ncbi.nlm.nih.gov/nuccore/MW036243.1/) is shown in FIG. 58.

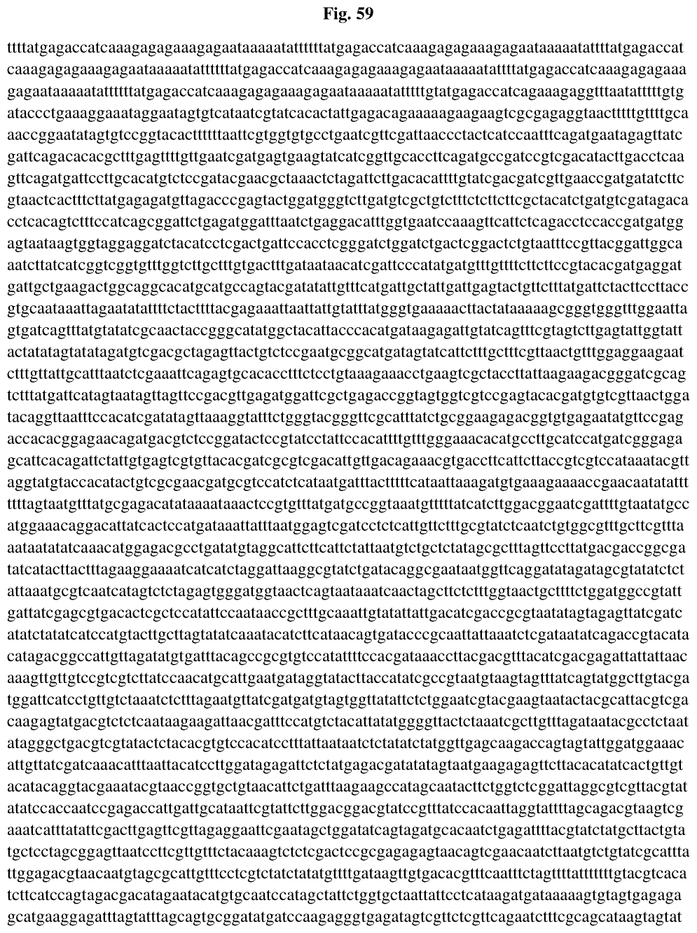

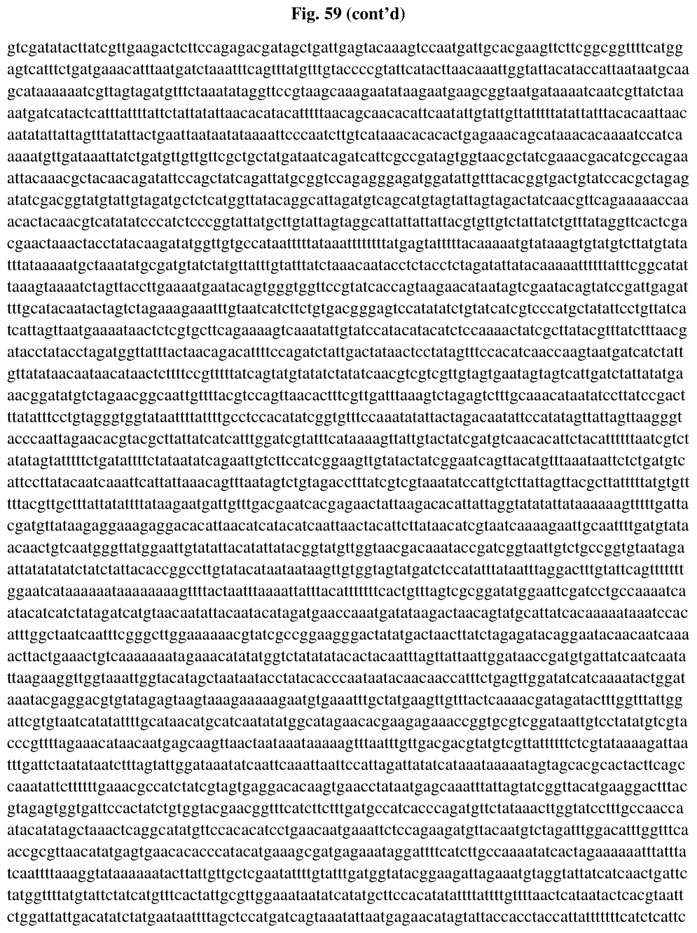

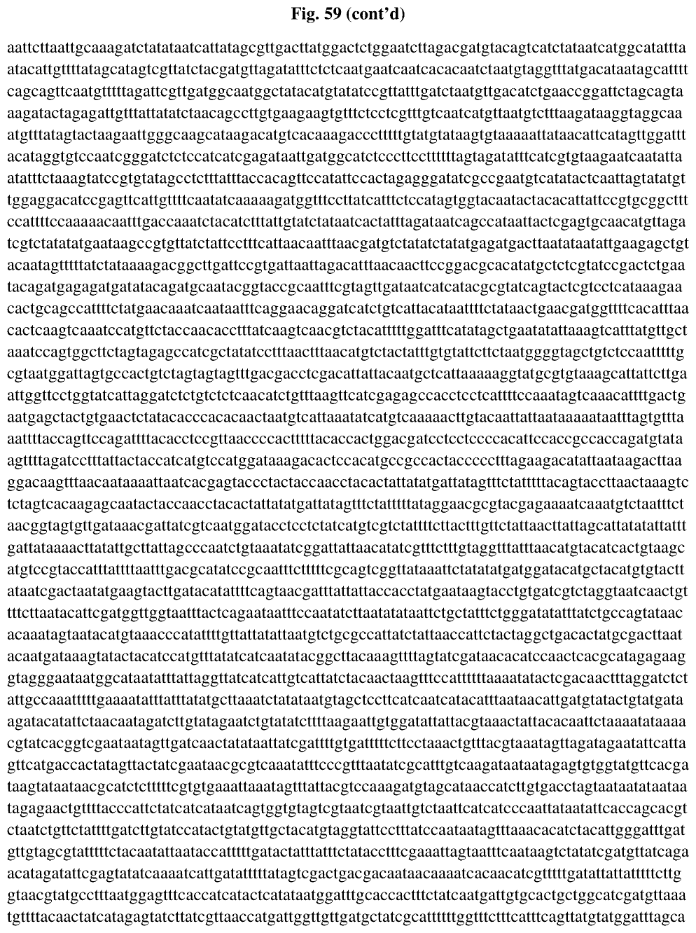

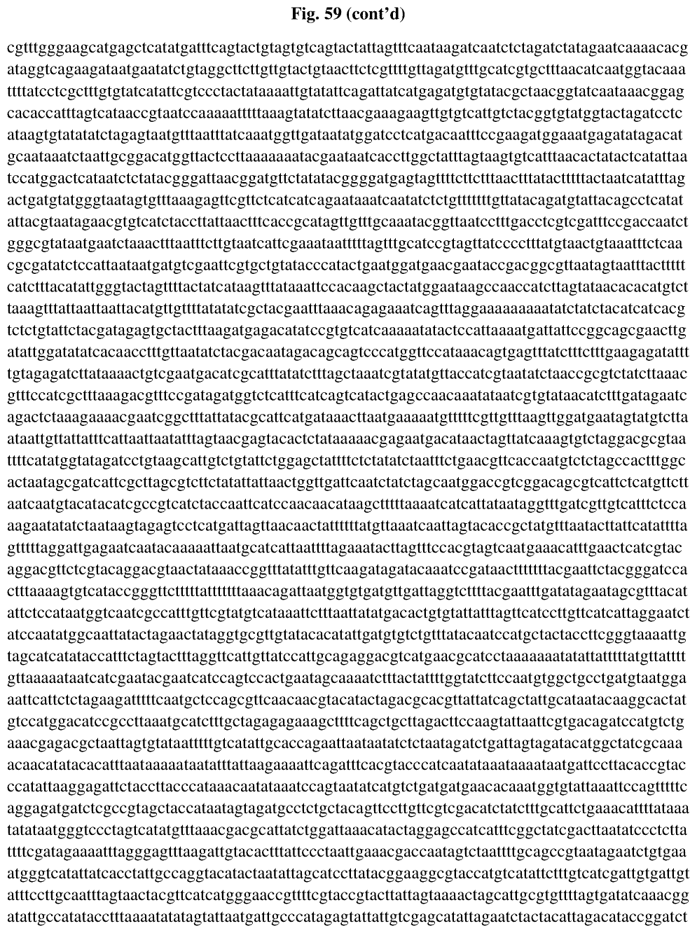

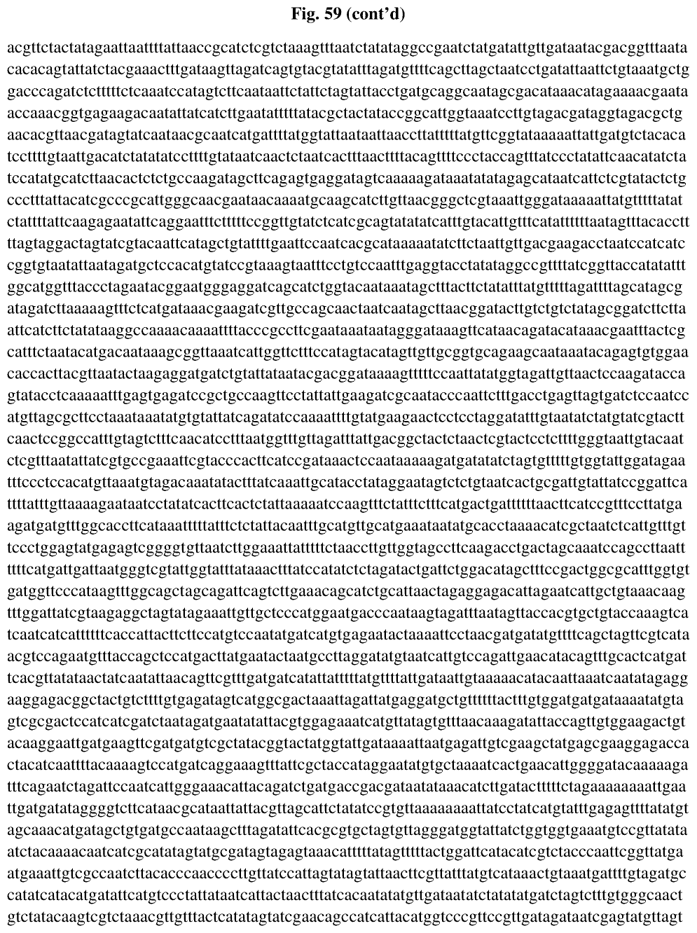

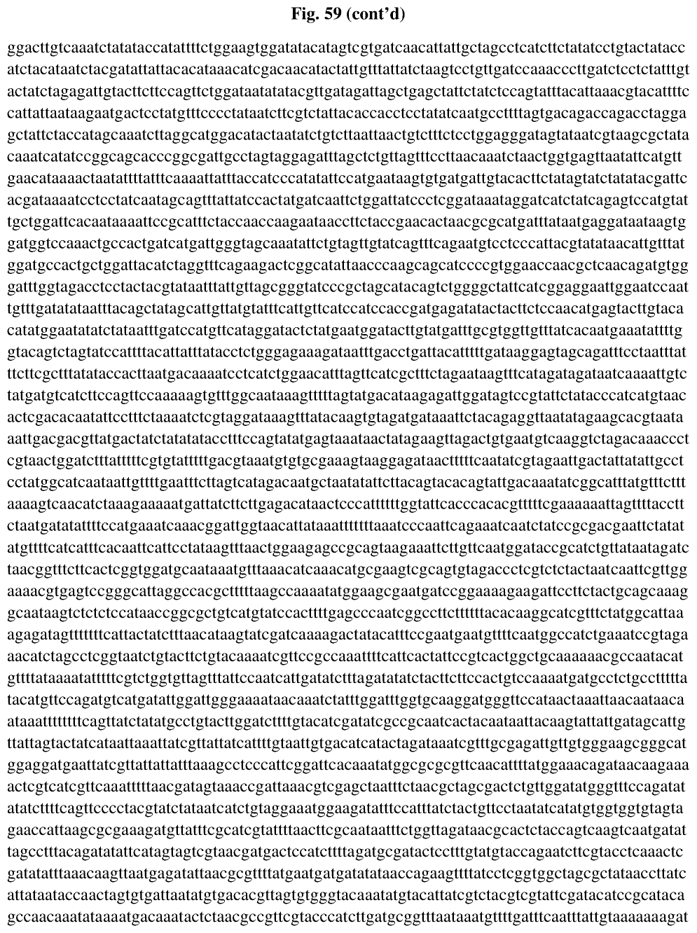

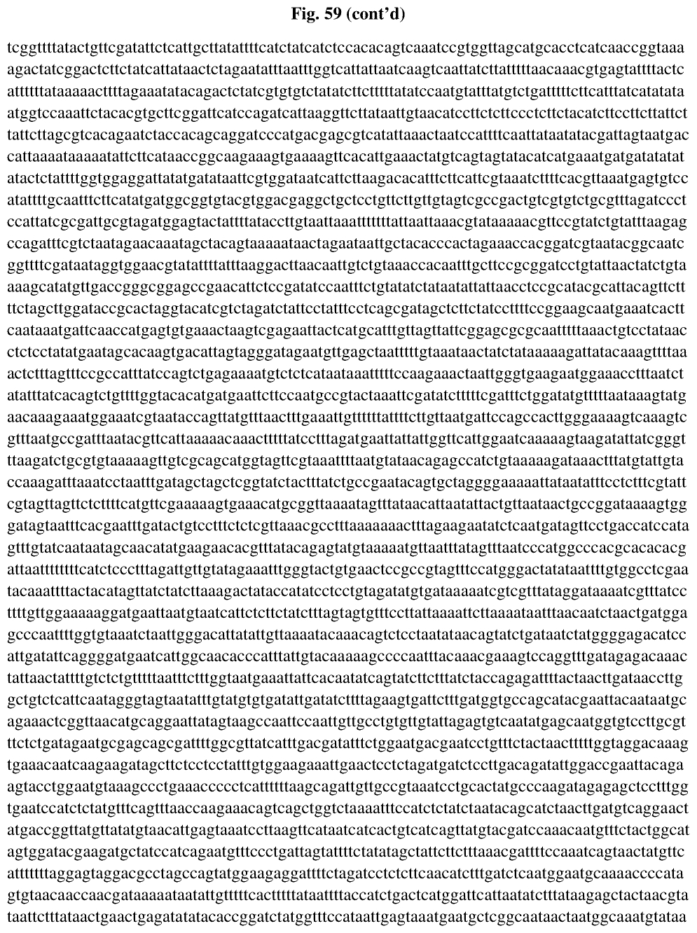

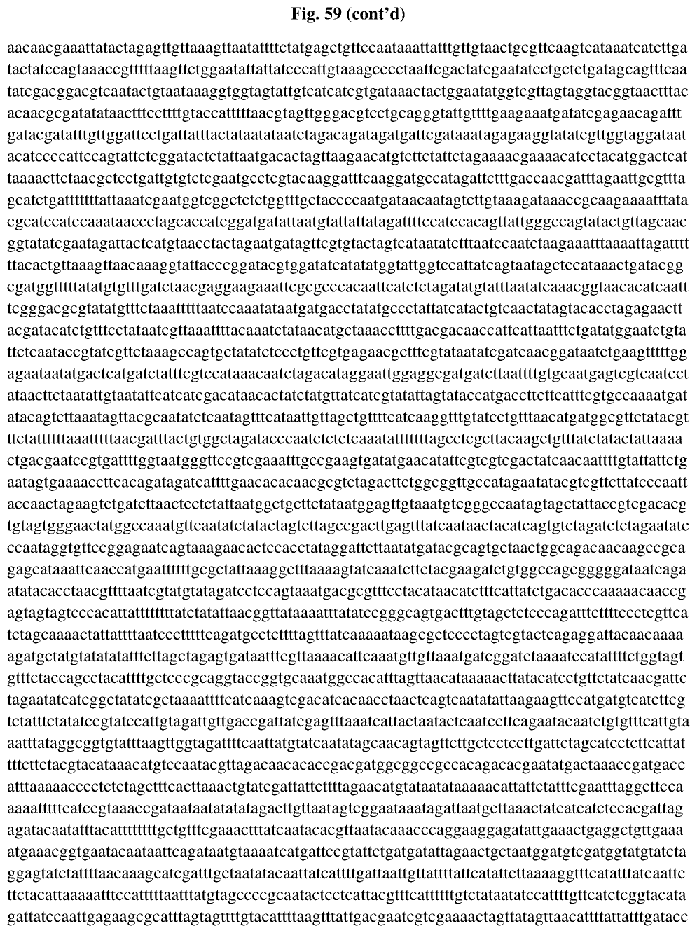

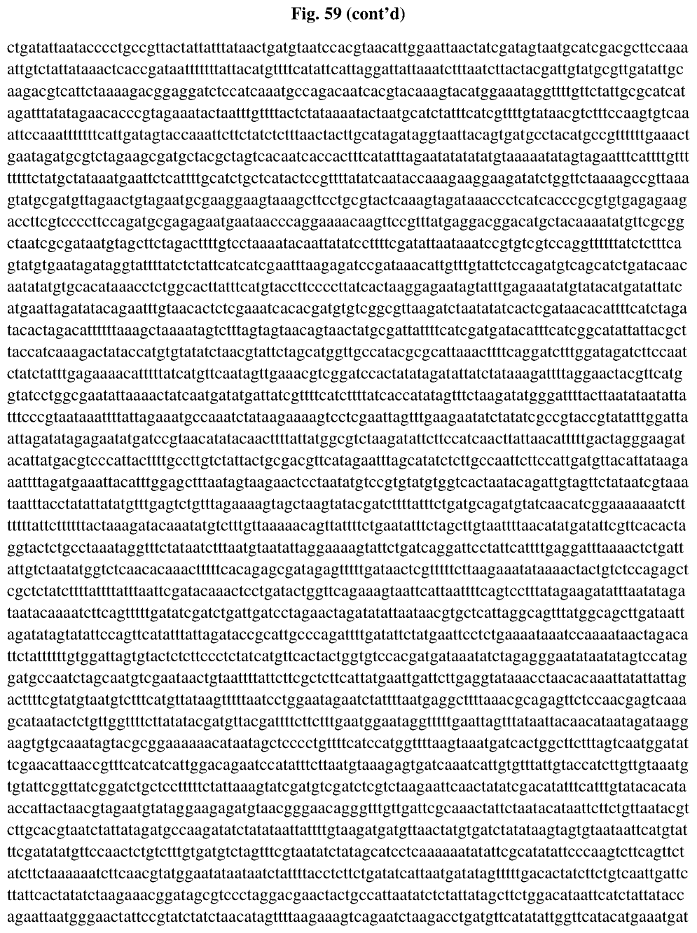

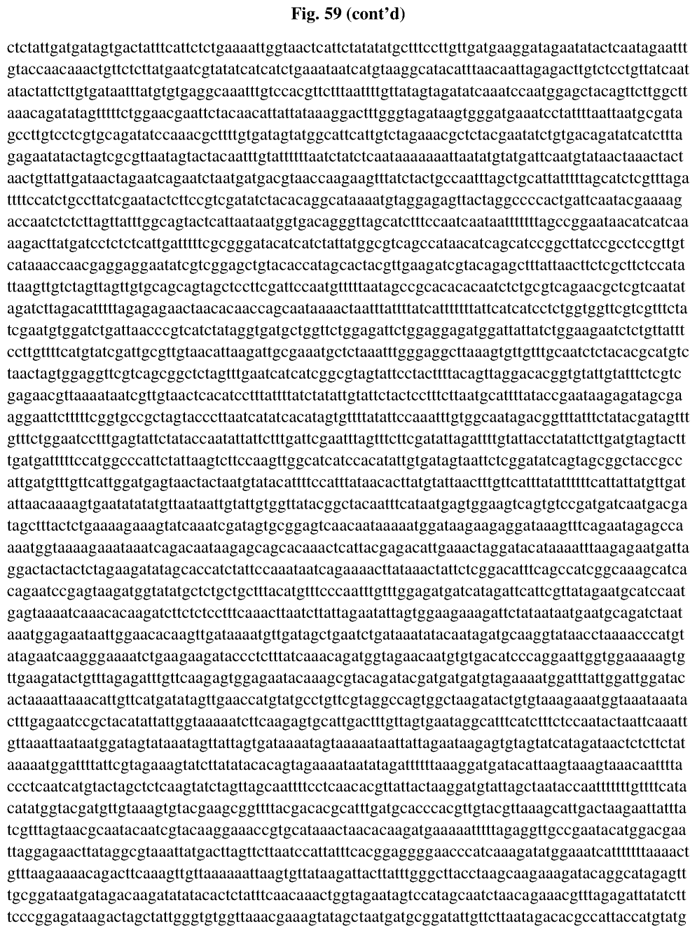

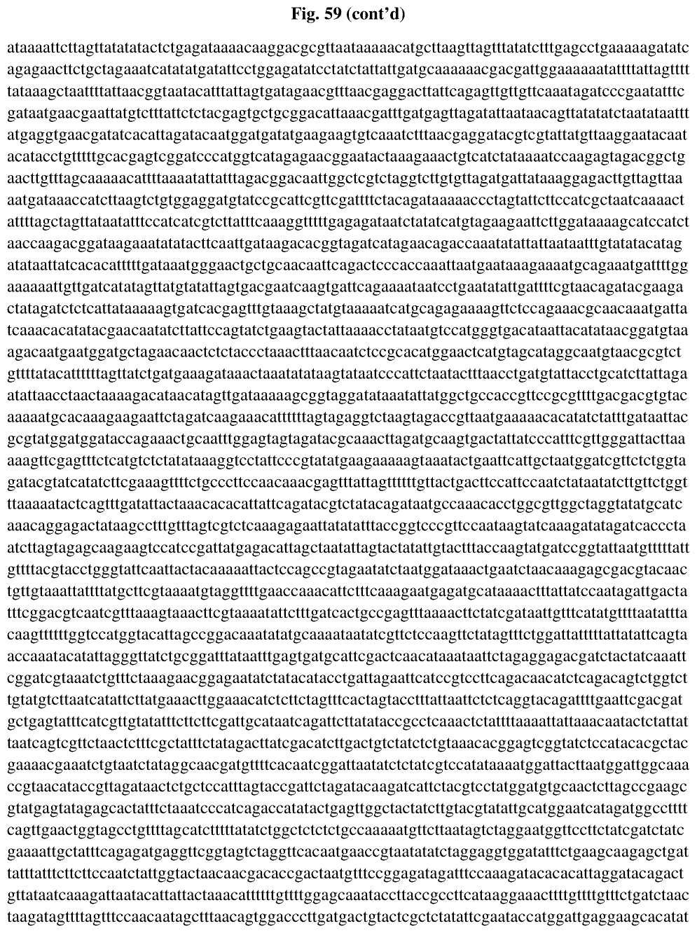

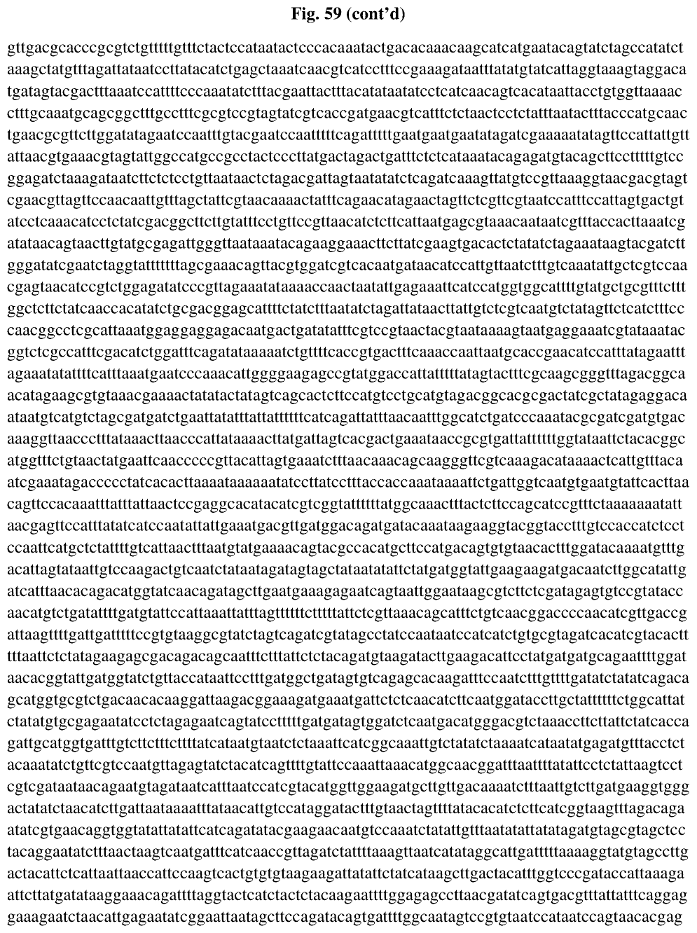

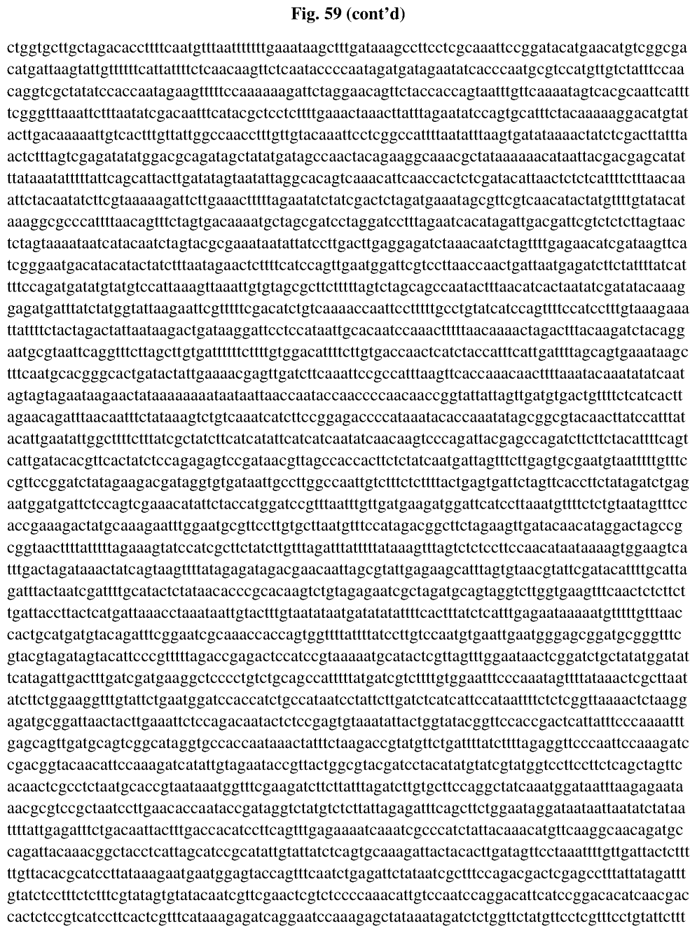

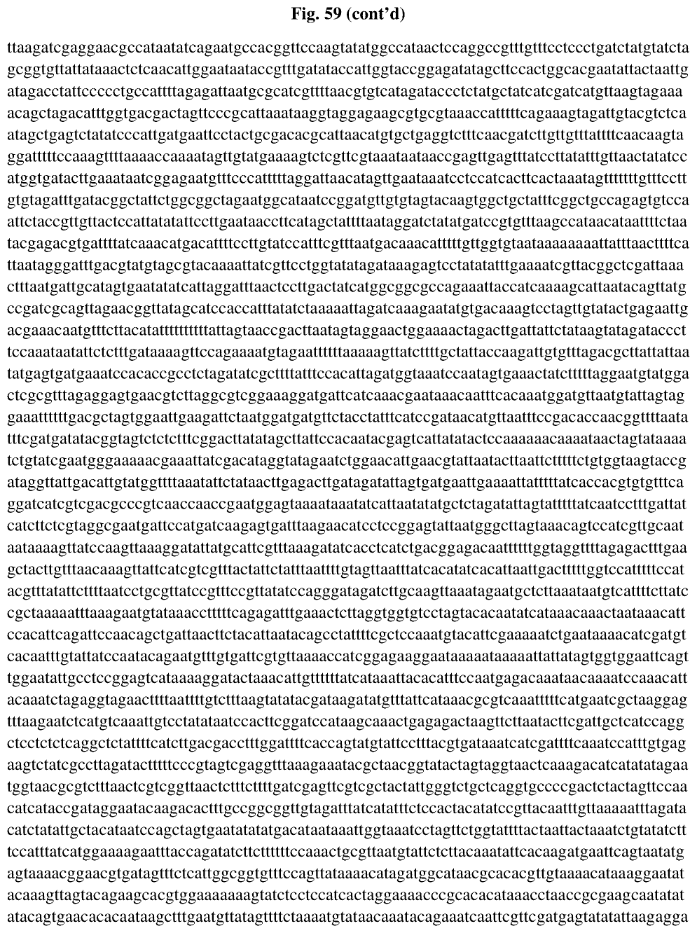

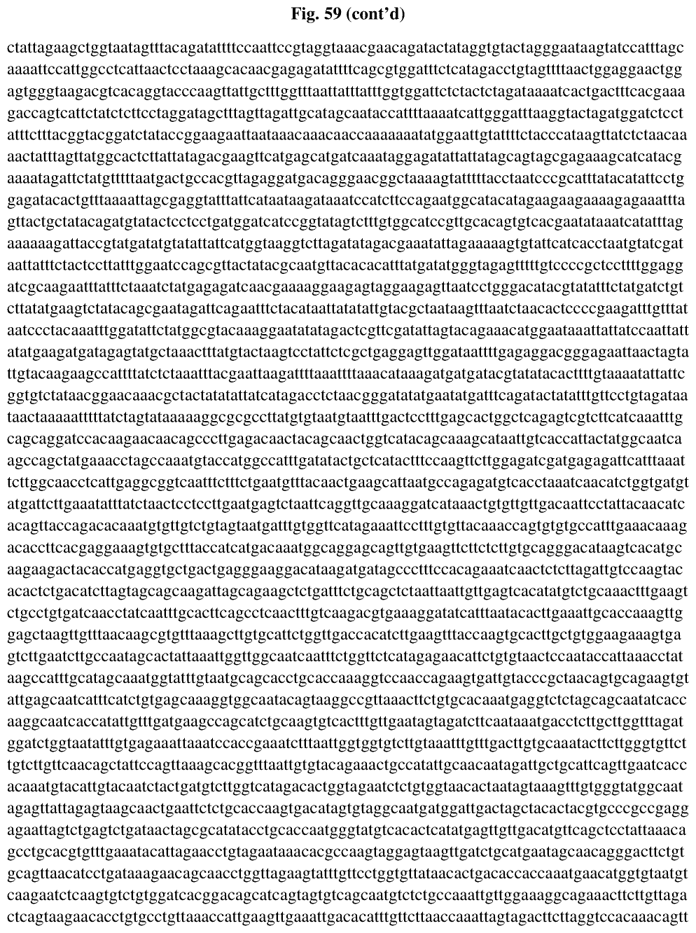

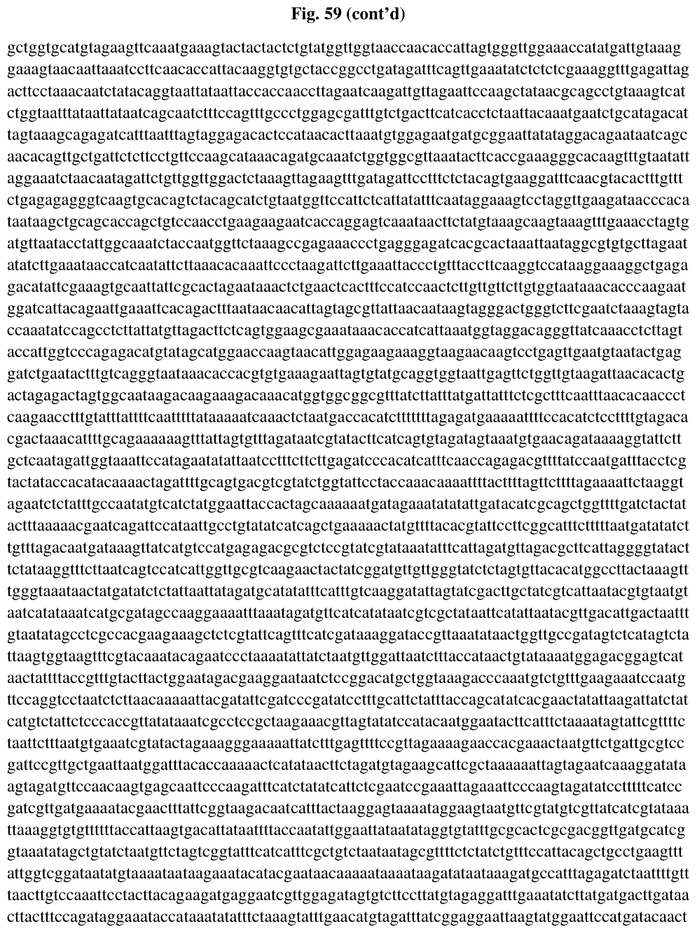

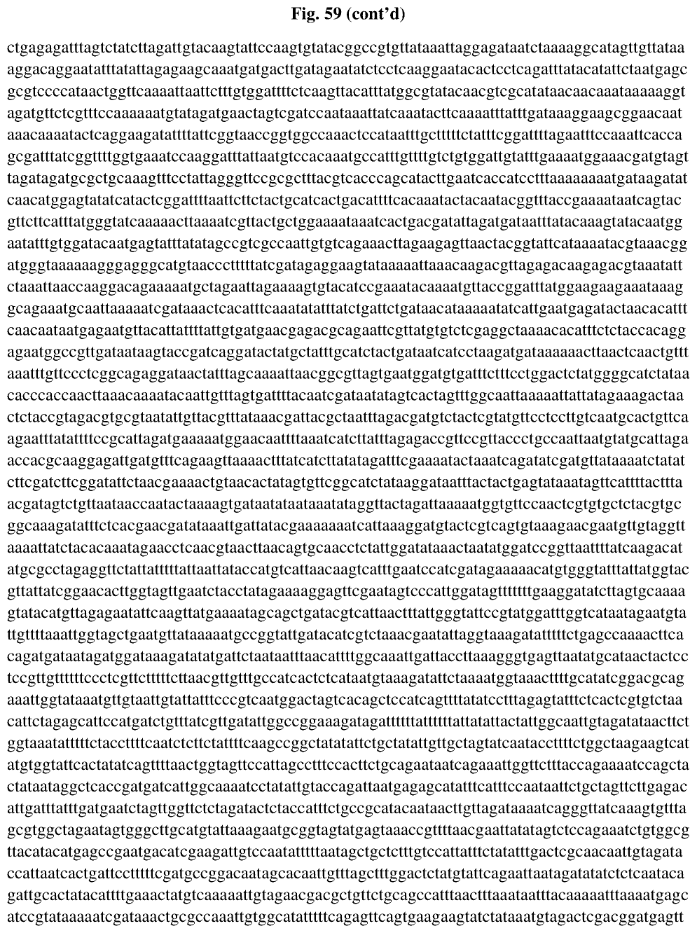

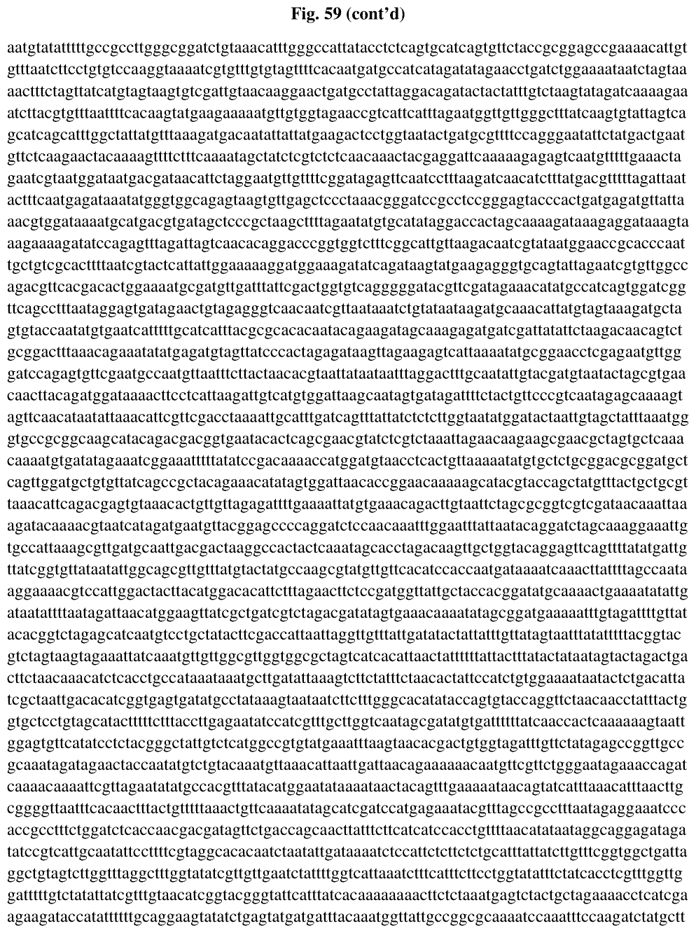

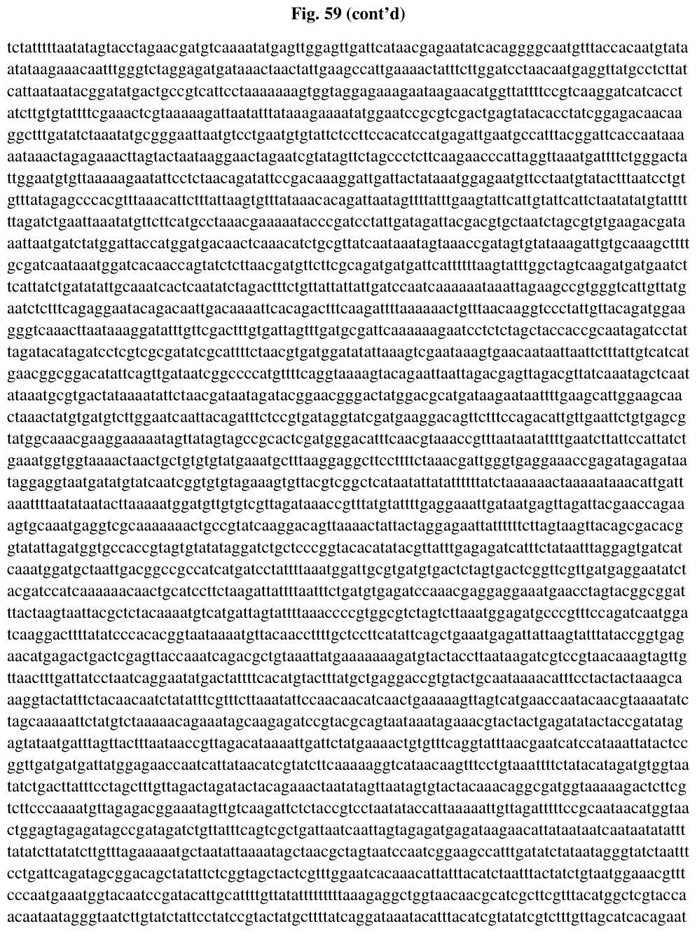

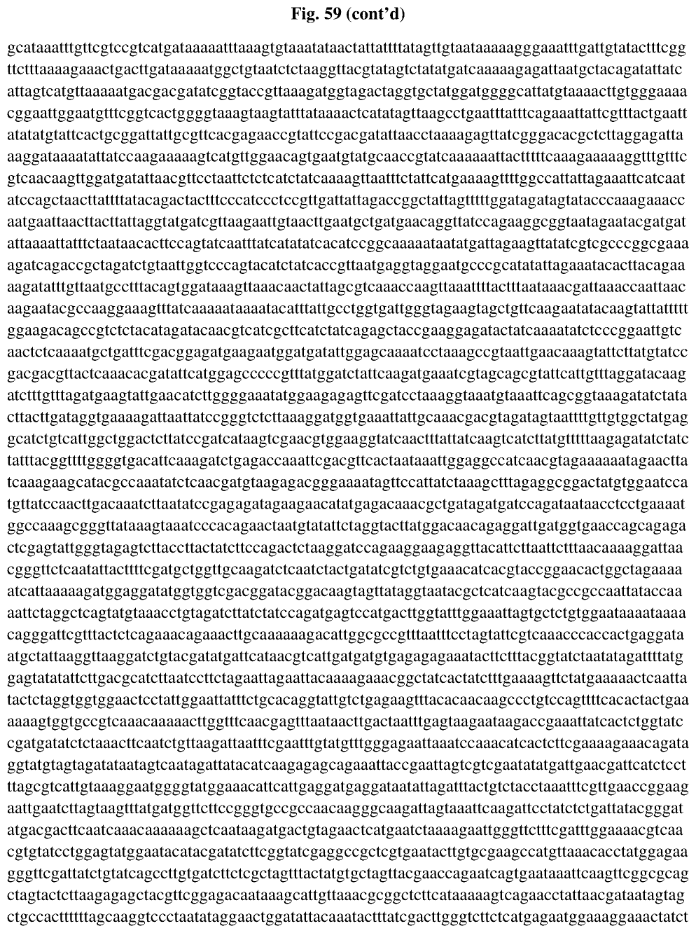

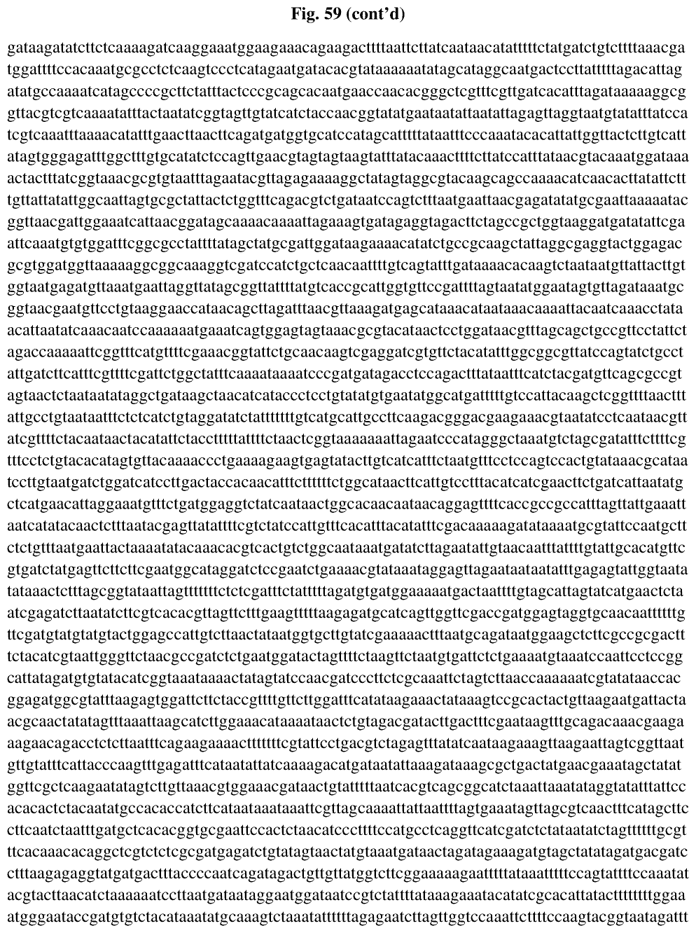

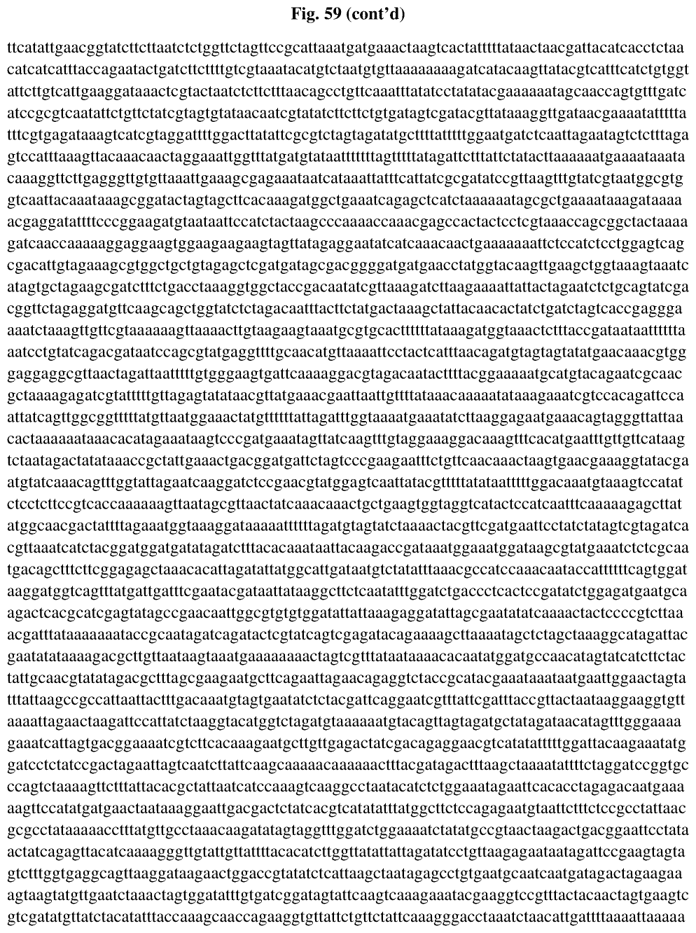

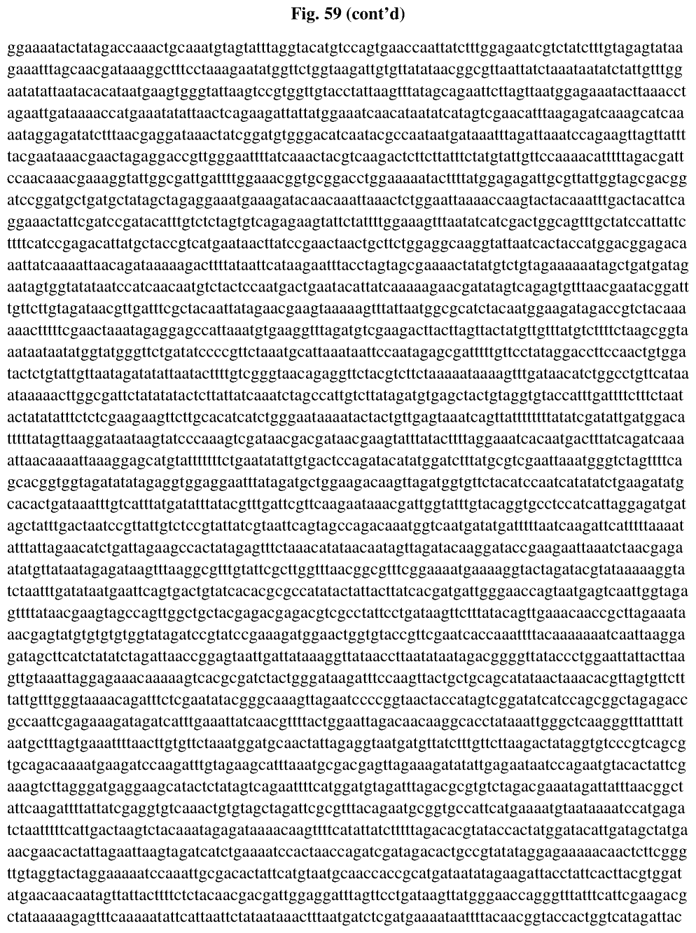

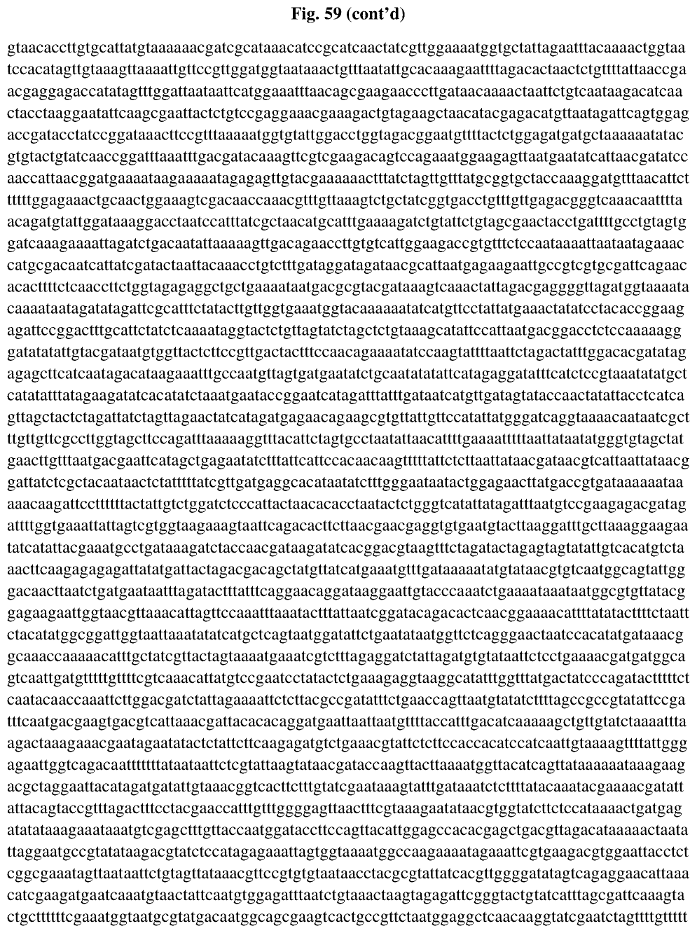

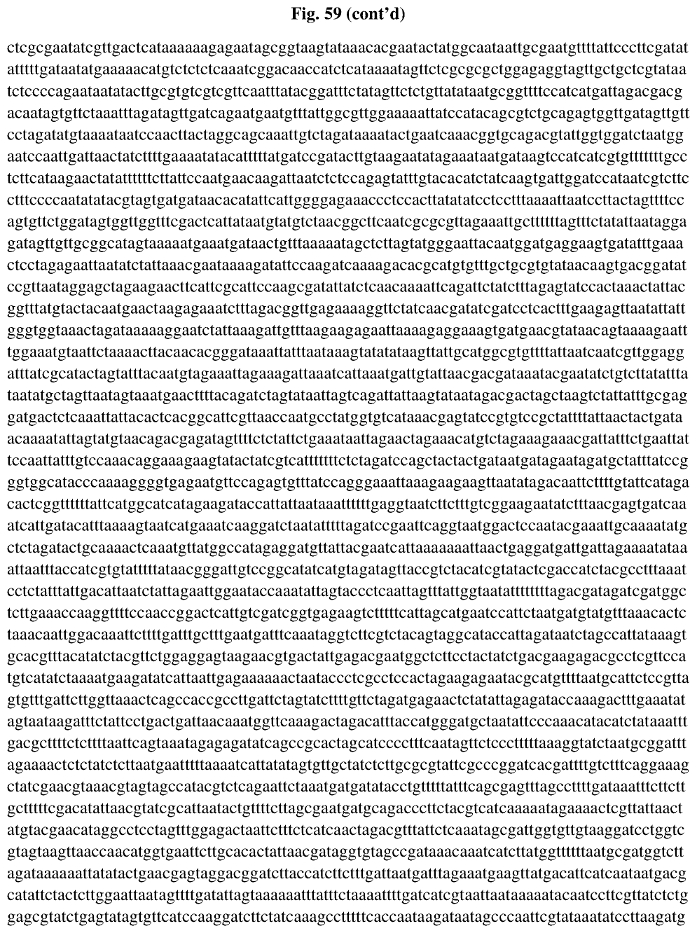

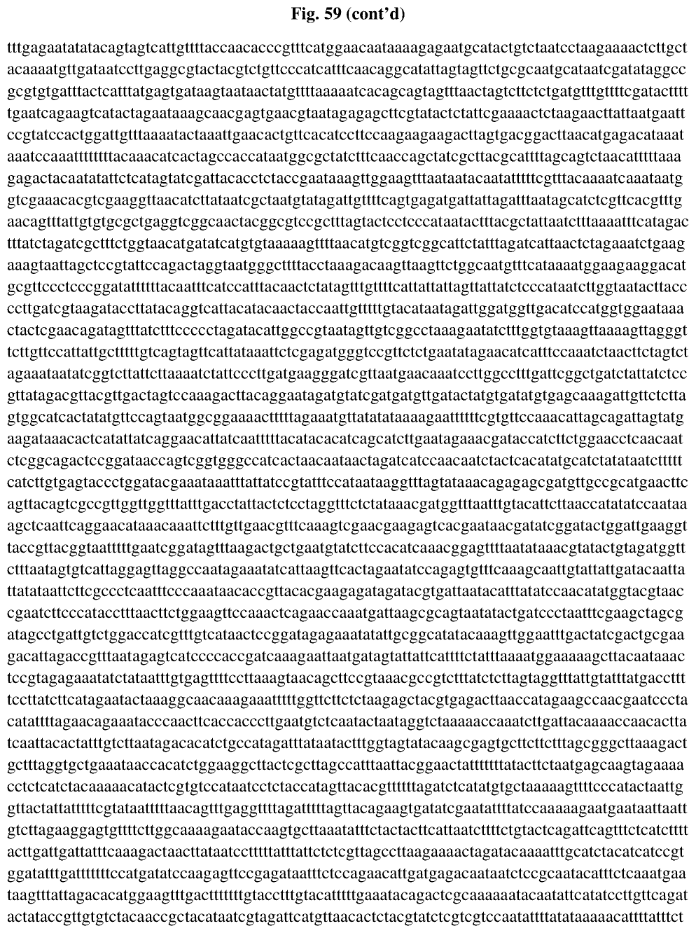

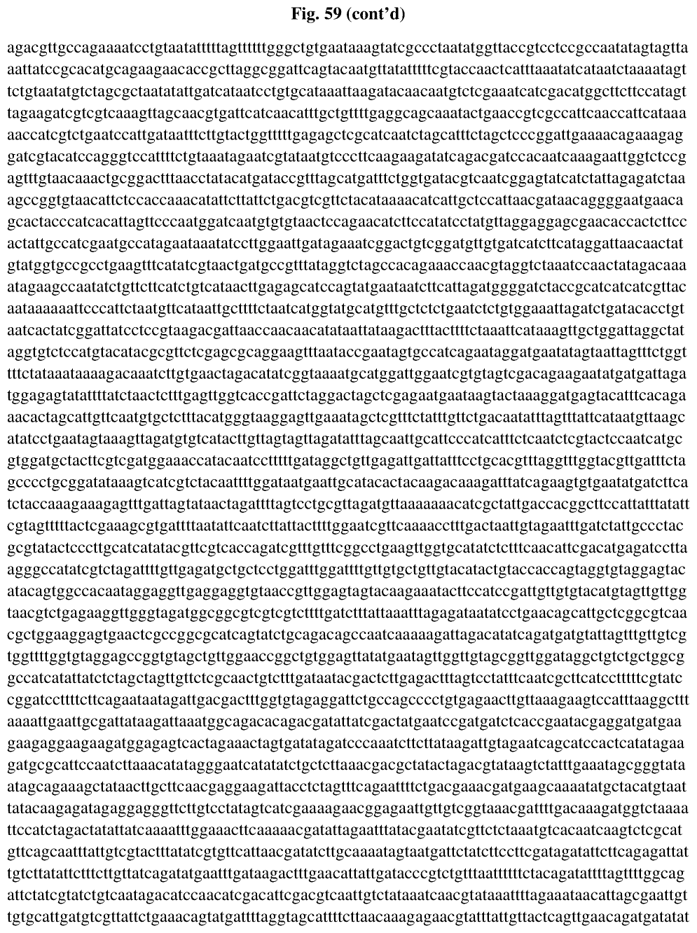

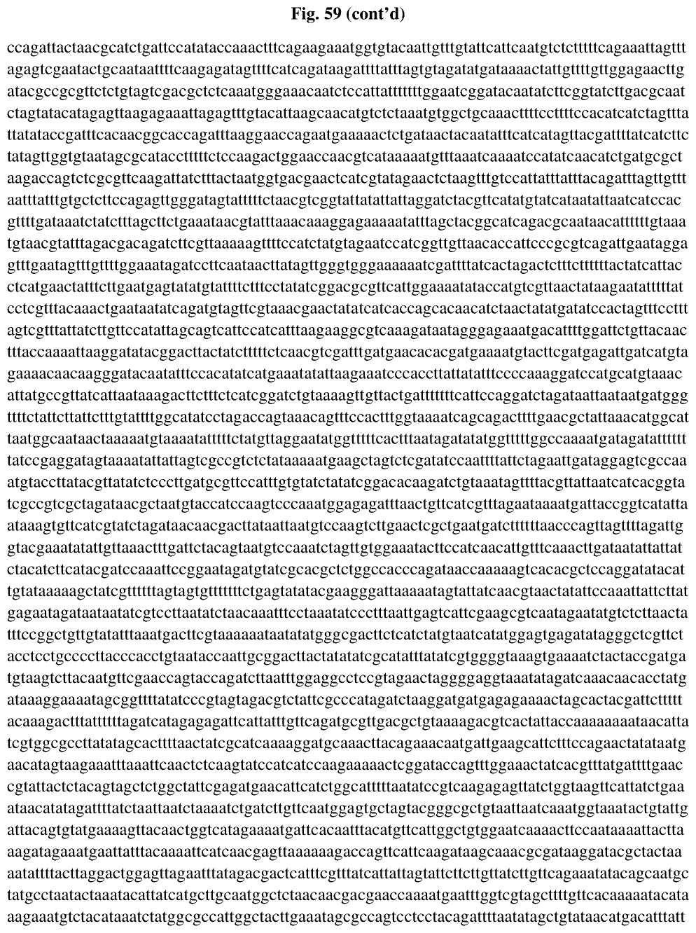

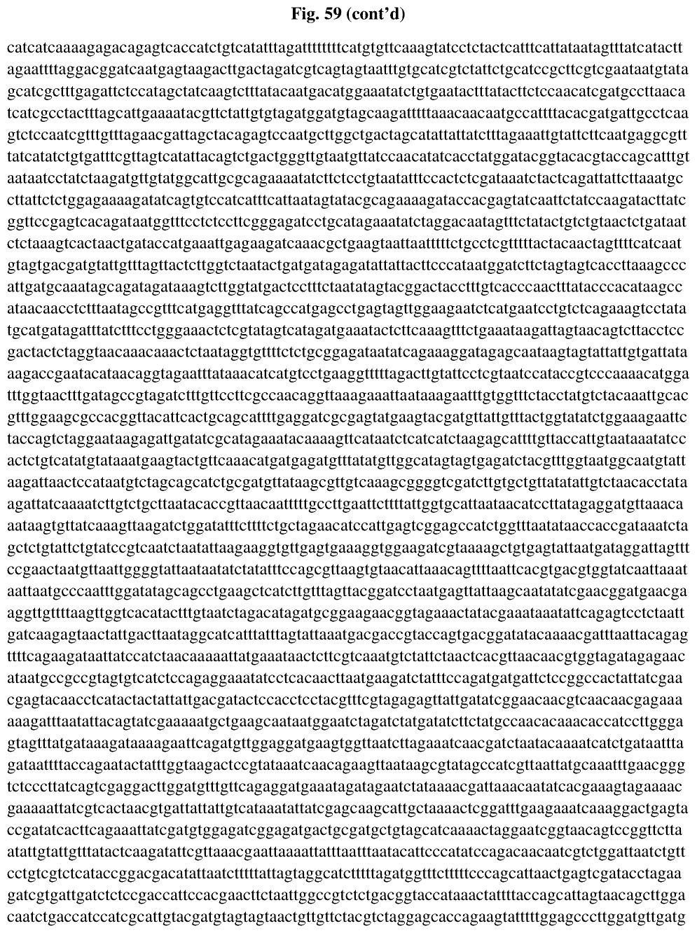

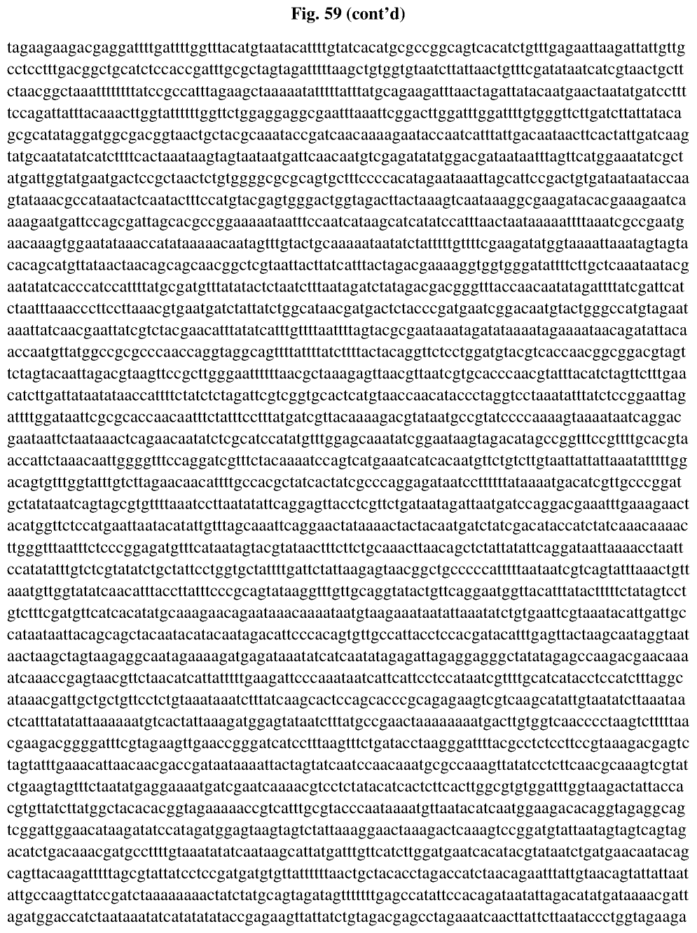

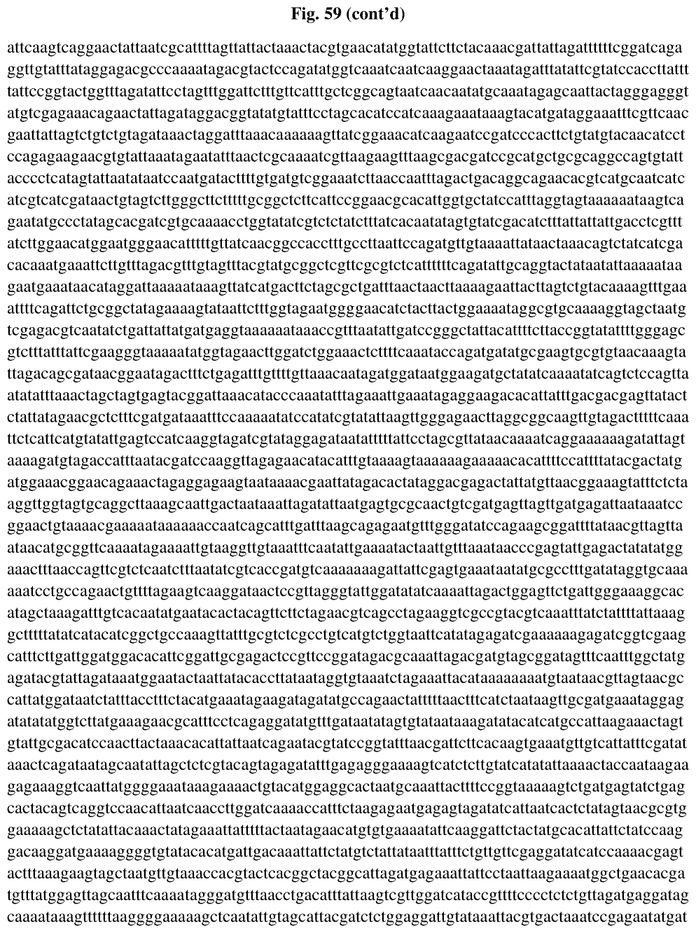

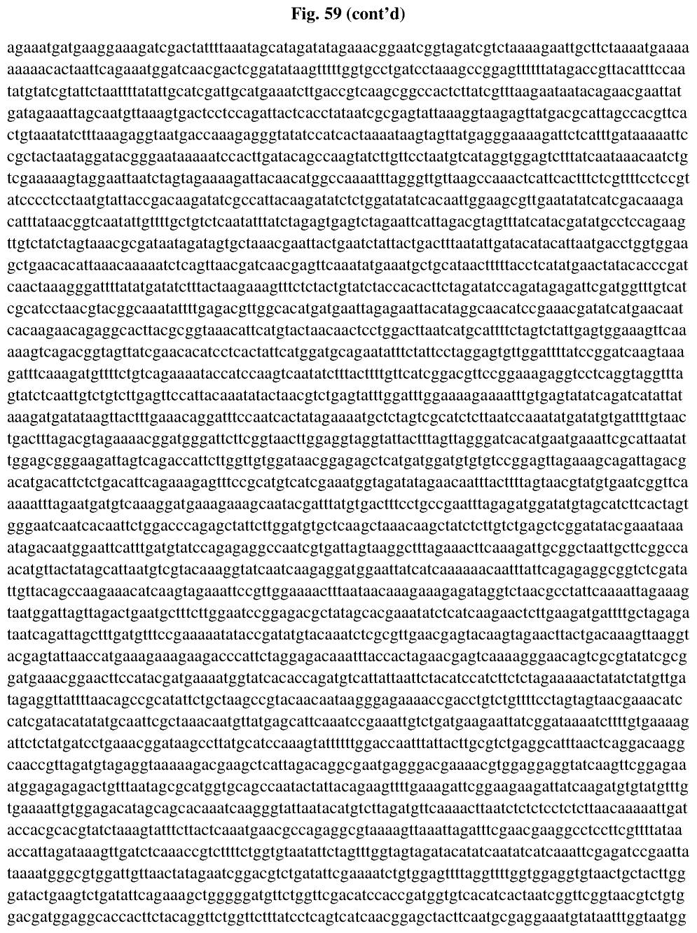

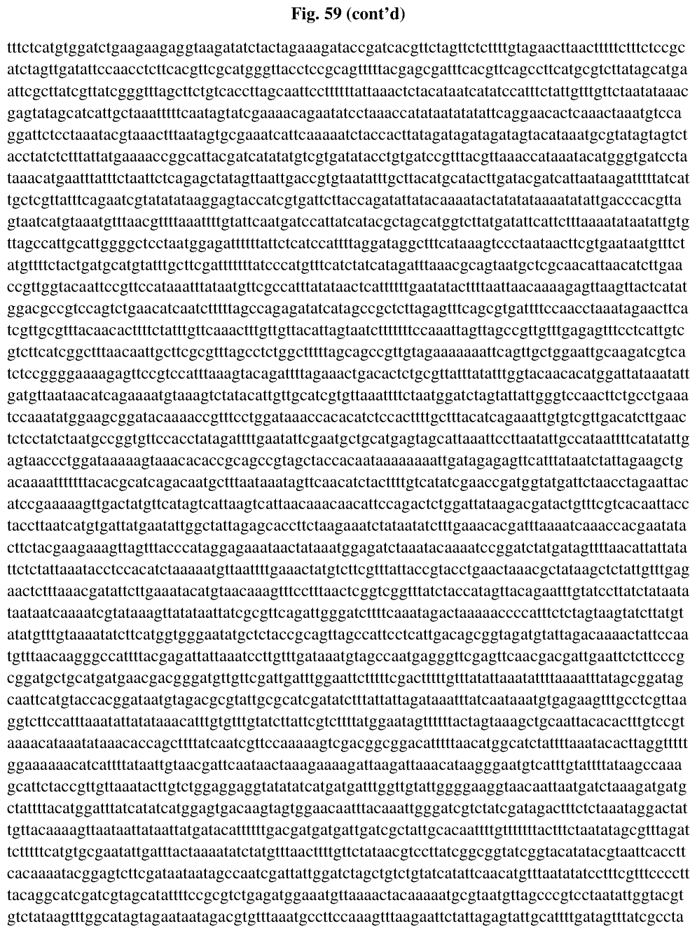

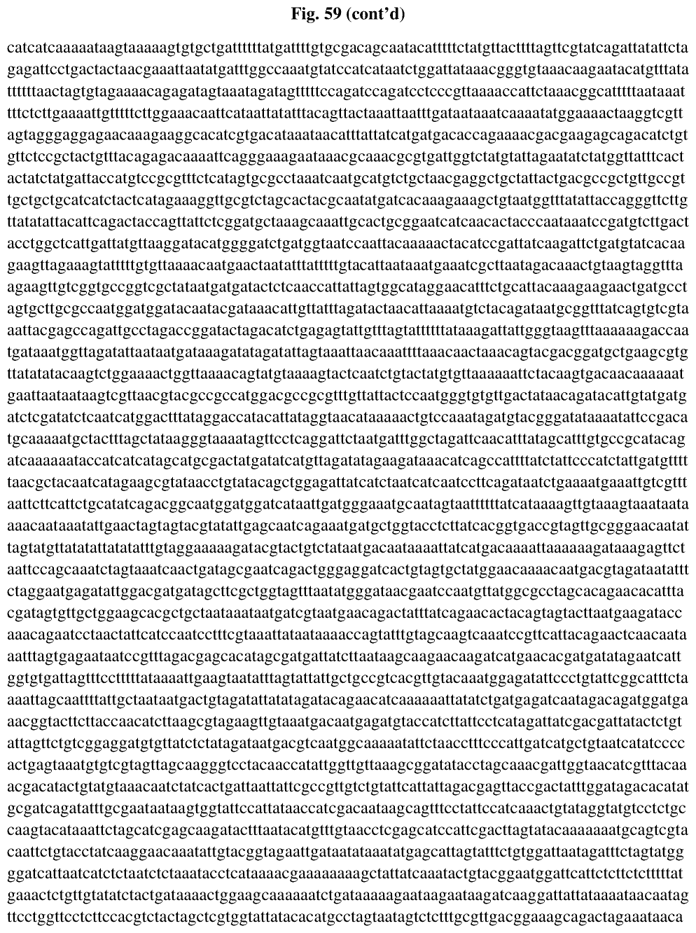

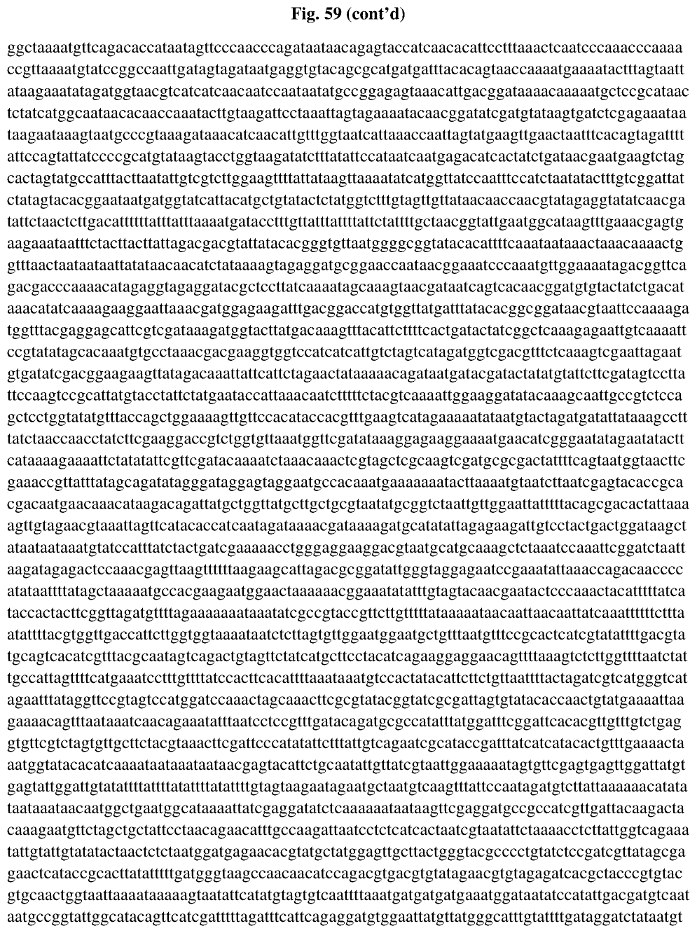

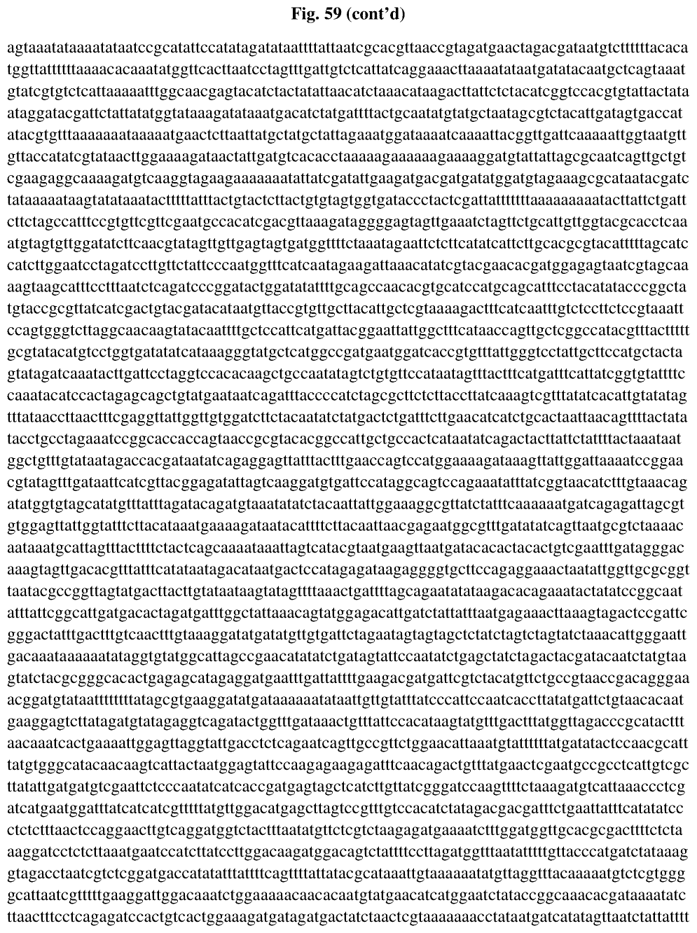

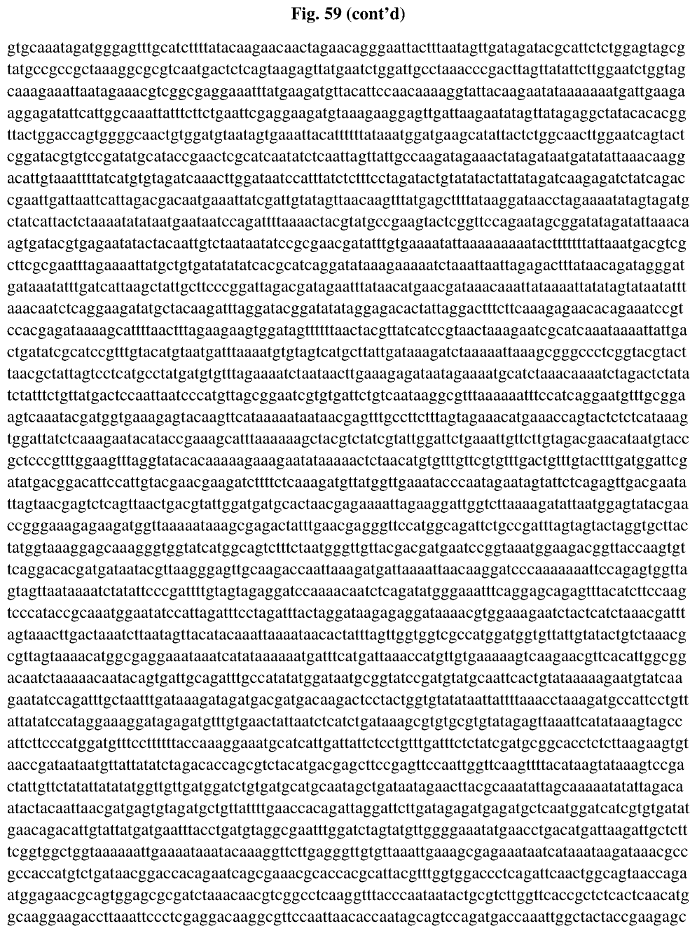

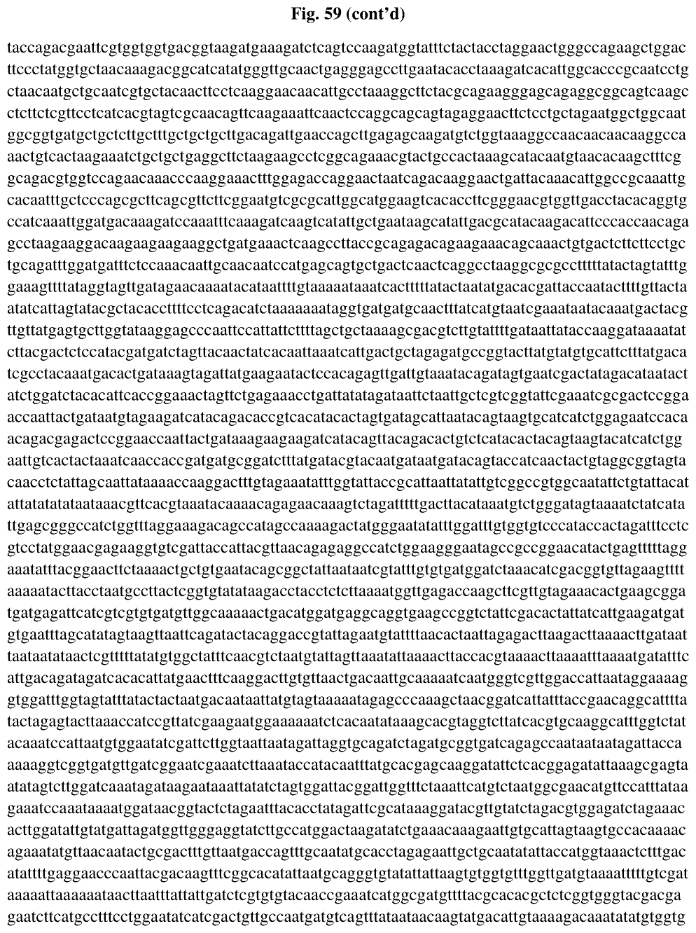

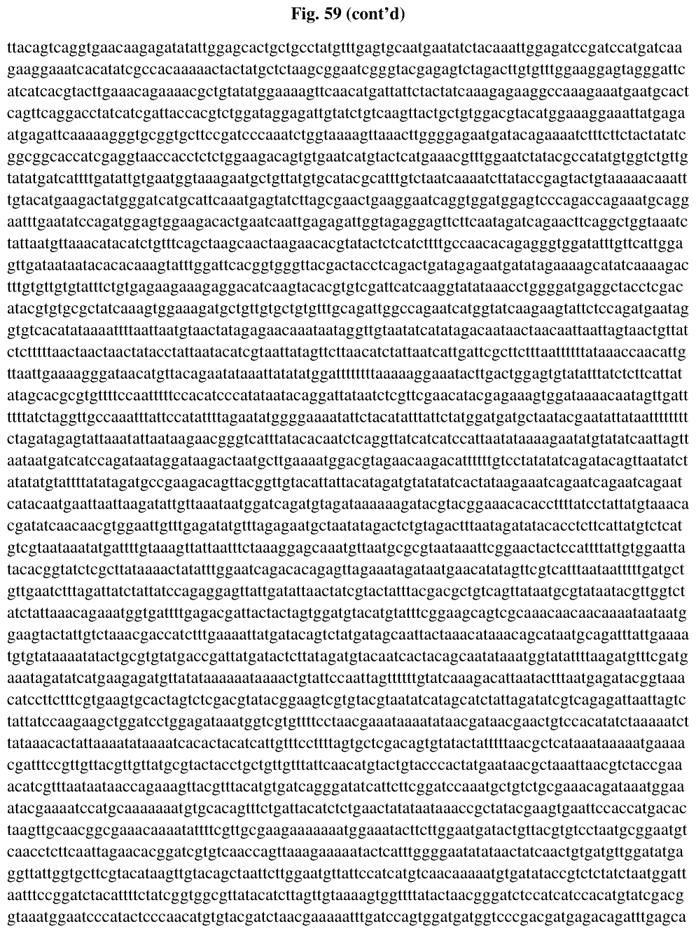

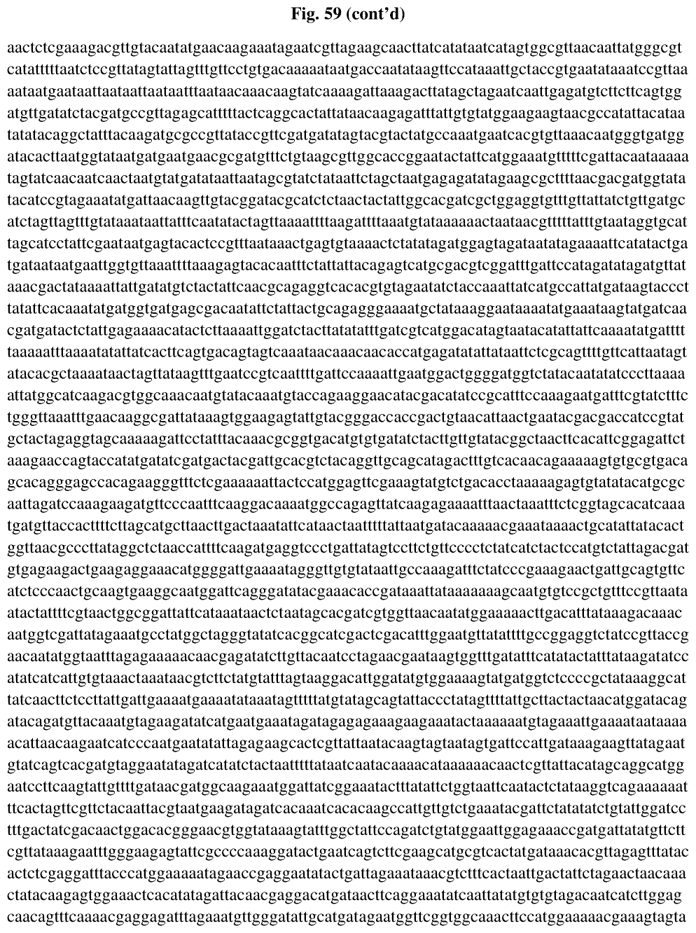

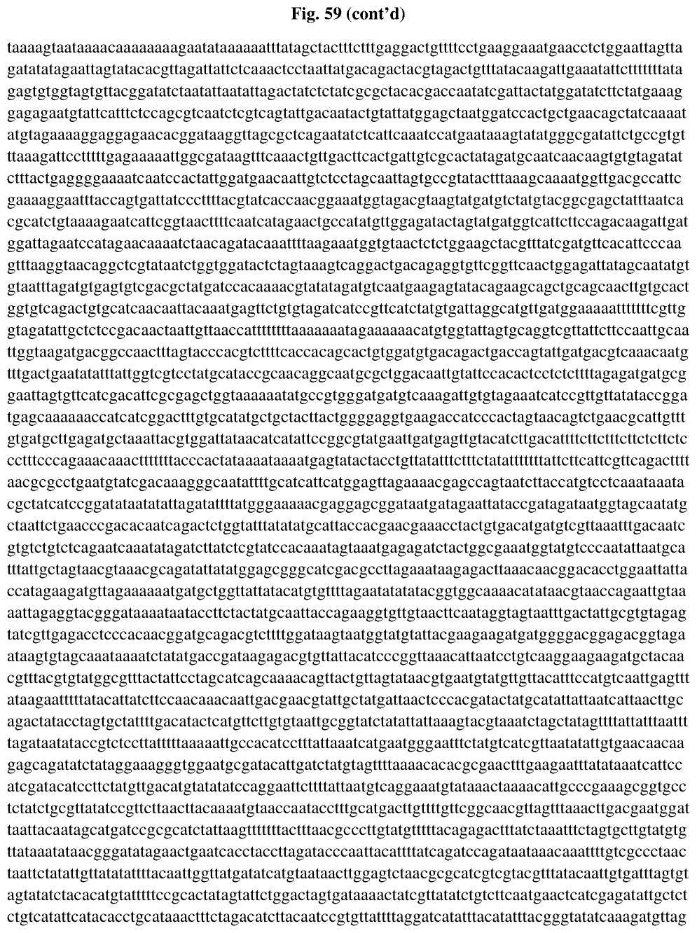

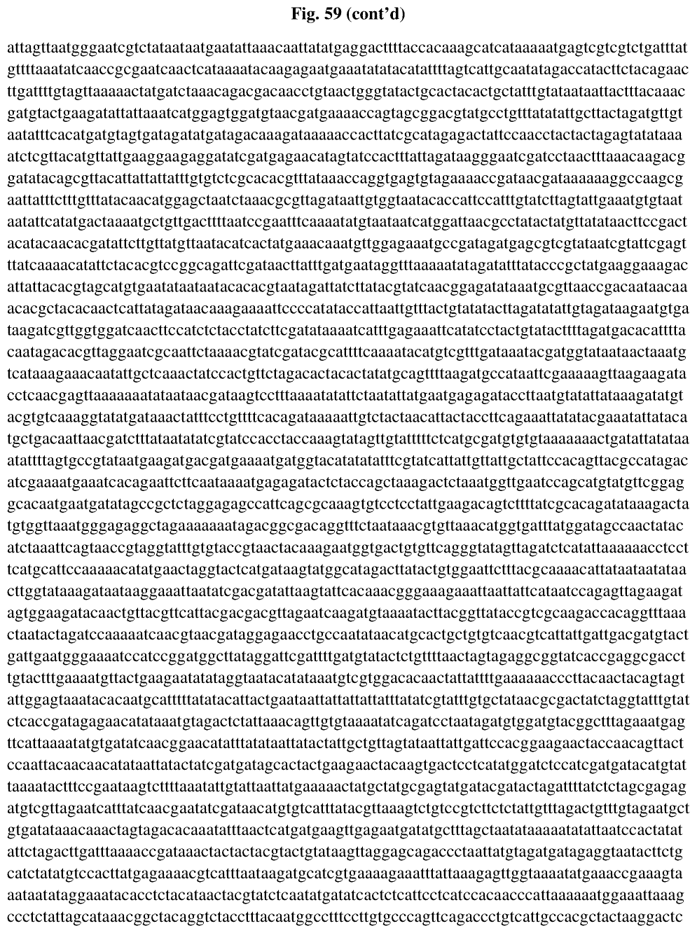

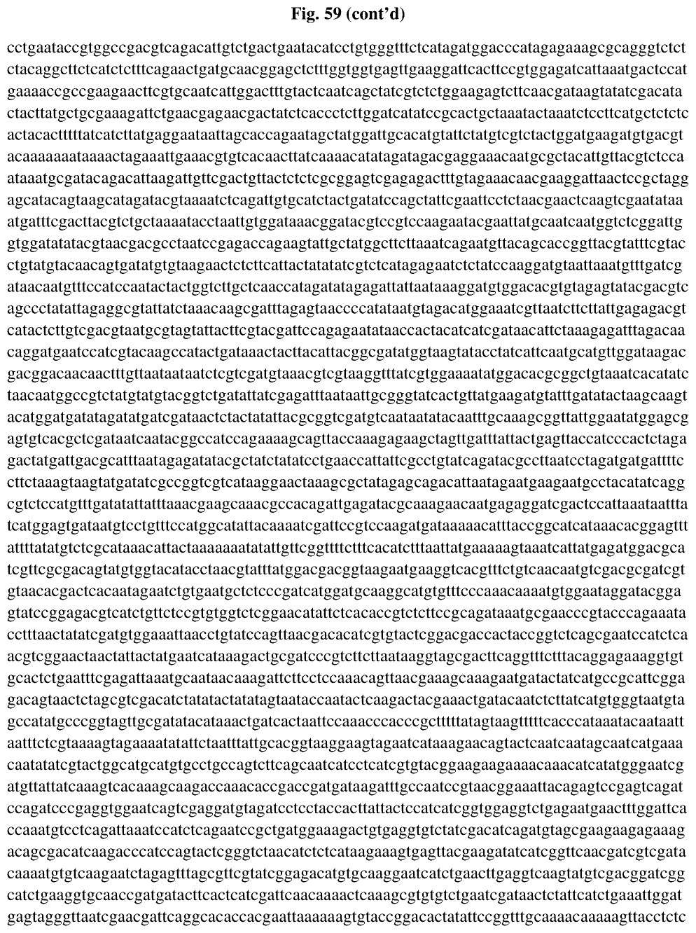

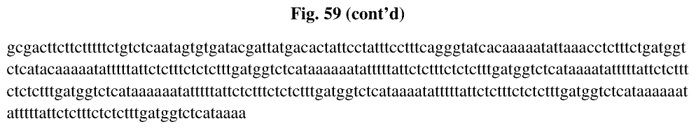

In certain embodiments, the sequence of sMVA-S/N (deposited with NCBI under Accession No. MW030460, www.ncbi.nlm.nih.gov/nuccore/MW030460.1/) is shown in FIG. 59.

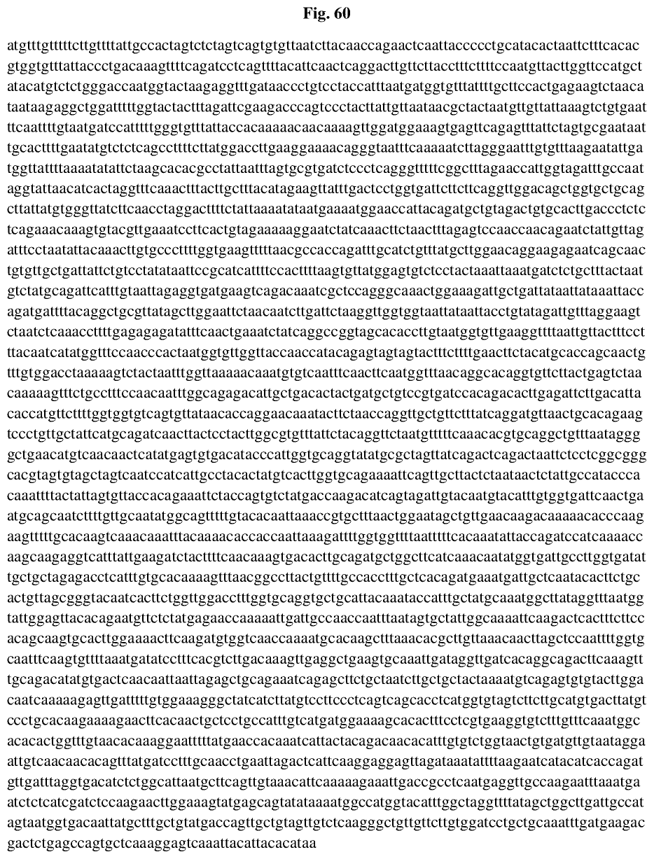

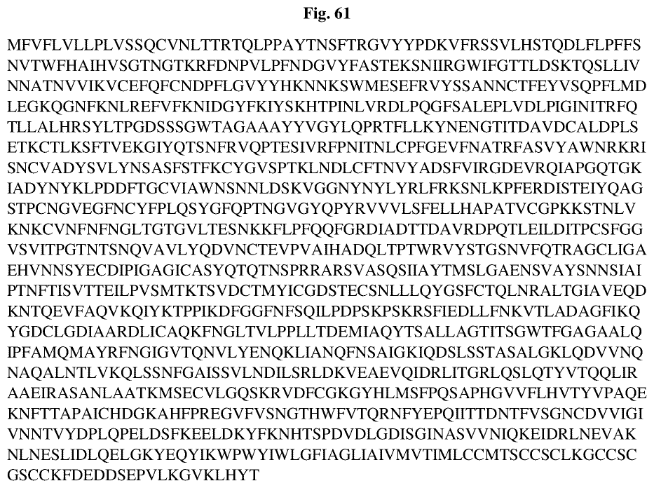

As disclosed herein, sequences of various SARS-CoV-2 antigens can be inserted in the sMVA vector to obtain the vaccine composition. The sequences of some antigens used herein are disclosed as follows. In one embodiment, the Spike (S) antigen sequence is based on the genome sequence of the NCBI SARS-CoV-2 reference strain (#NC_045512), isolated Wuhan-Hu-1, which encodes a S protein comprising 1273 amino acids. The DNA sequence/open reading frame (ORF) (5′ to 3′ end) is shown in FIG. 60, and the encoded protein sequence (N- to C-terminus) is shown in FIG. 61.

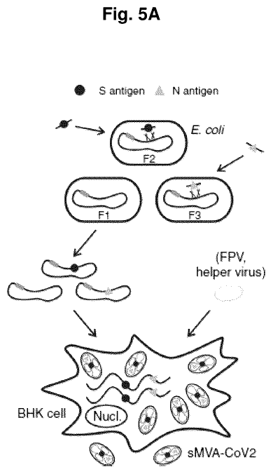

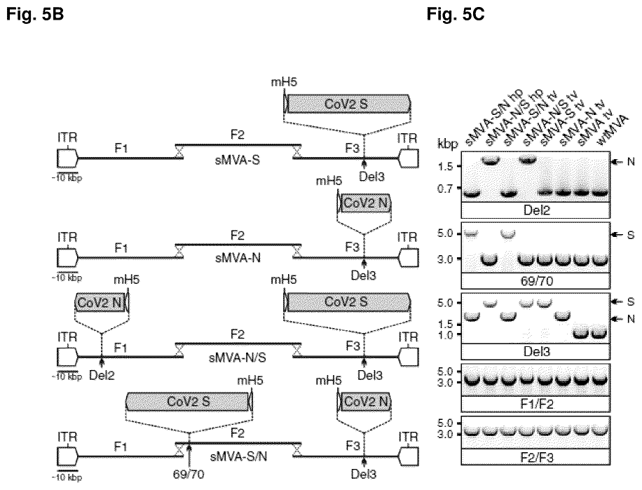

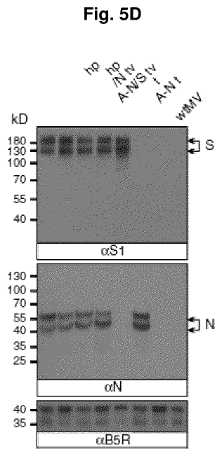

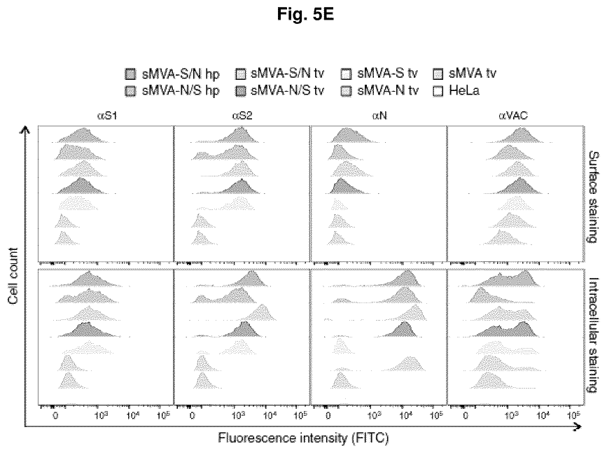

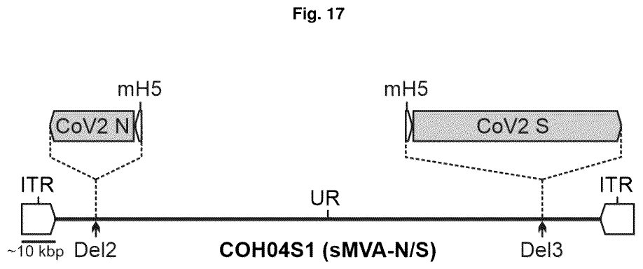

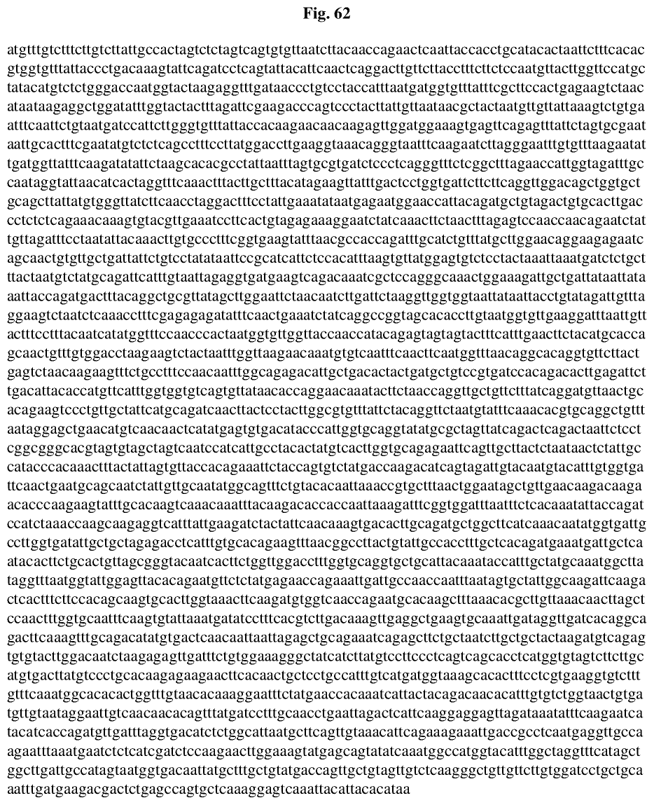

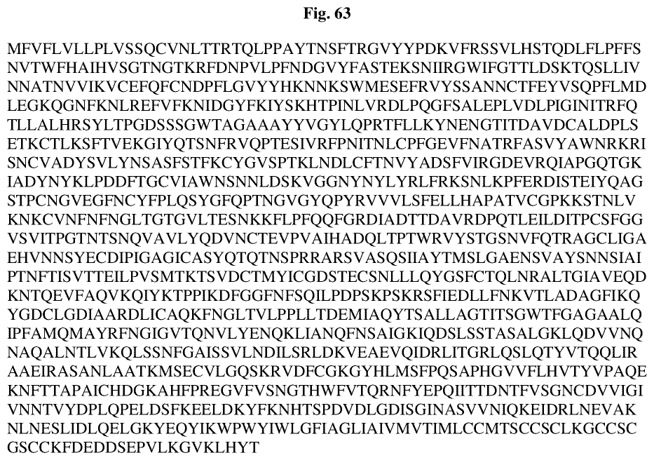

In another example, the SARS-CoV-2 S antigen sequence is based on the Wuhan-Hu-1 reference strain (#NC_045512) and optimized for stability in vaccinia by silent codon alteration to avoid 4 or more of the same nucleotides in consecutive order. The DNA/sequence/ORF (5′ to 3′ end) used in COH04SL1 (a.k.a., construct C15 and illustrated as sMVA-S in FIG. 5), COH04SL3 (a.k.a., construct C35 and illustrated as sMVA-N/S in FIG. 5), and COH04SL4 (a.k.a., construct C46 and illustrated as sMVA-S/N in FIG. 5), as well as in the clinical construct COH04S1 (FIG. 17, COH04S1 was derived from the C35 sMVA-N/S vaccine construct (FIG. 5) as illustrated in FIG. 57), is shown in FIG. 62, and the encoded protein sequence (N- to C-terminus) is shown in FIG. 63.

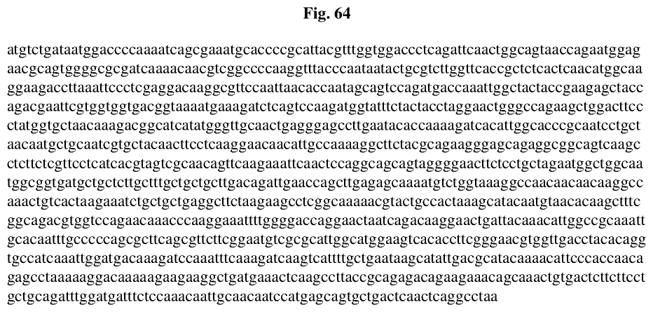

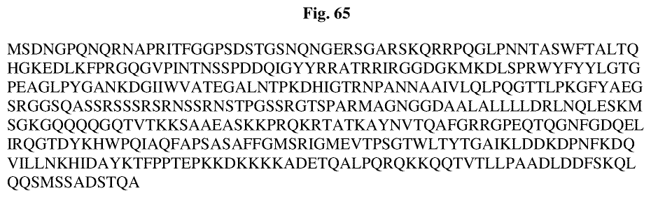

In one embodiment, the Nucleocapsid (N) antigen sequence is based on the genome sequence of the NCBI SARS-CoV-2 reference strain (#NC_045512), isolate Wuhan-Hu-1, which encodes a N protein composed of 419 amino acids. The DNA sequence/ORF (5′ to 3′ end) is shown in FIG. 64, and the encoded protein sequence (N-to C-terminus) is shown in FIG. 65.

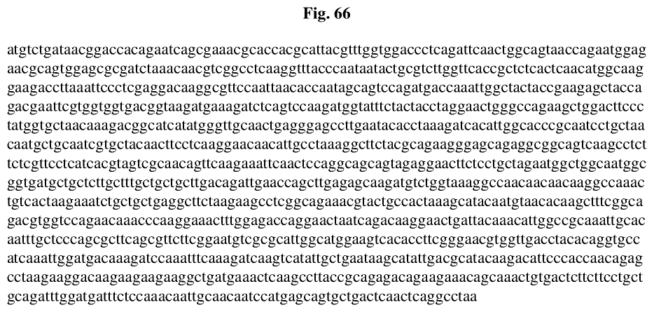

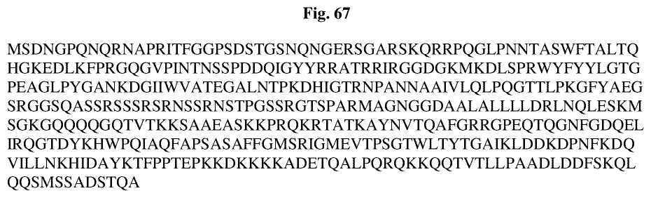

In another example, the SARS-CoV-2 N antigen sequence is based on the Wuhan-Hu-1 reference strain (#NC_045512) and optimized for stability in vaccinia by silent codon alteration to avoid 4 or more of the same nucleotides in consecutive order. The DNA sequence/ORF (5′ to 3′ end) used in COH04SL2 (a.k.a., construct C13 and illustrated as sMVA-N in FIG. 5), COH04SL3 (a.k.a., construct C35 and illustrated as sMVA-N/S in FIG. 5), and COH04SL4 (a.k.a., construct C46 and illustrated as sMVA-S/N in FIG. 5), as well as in the clinical construct COH04S1 (FIG. 17, COH04S1 was derived from the C35 sMVA-N/S vaccine construct (FIG. 5) as illustrated in FIG. 57), is shown in FIG. 66, and the encoded protein sequence (N- to C-terminus) is shown in FIG. 67.

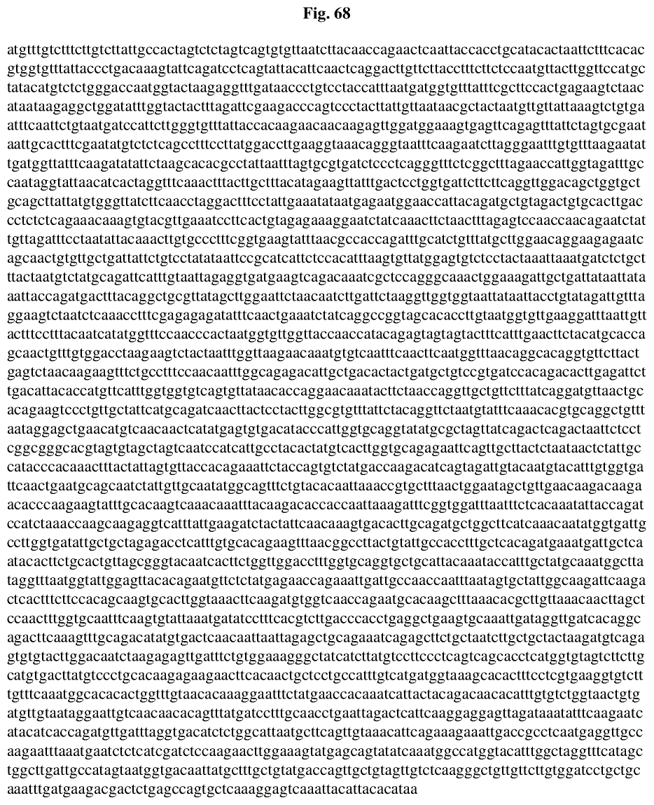

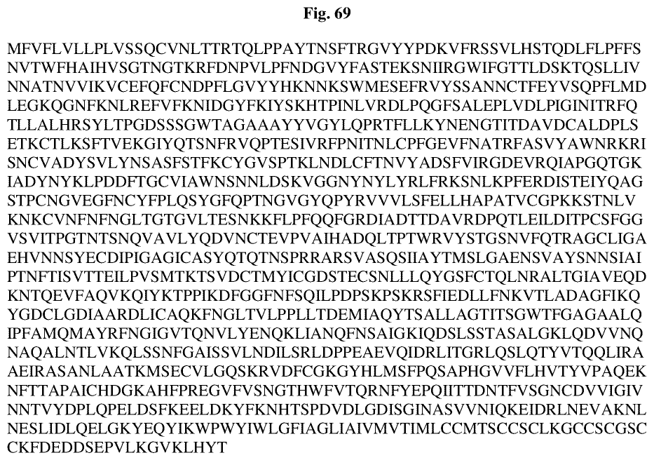

In another embodiment the SARS-CoV-2 S antigen sequence is modified to encode for a prefusion stabilized S antigen with 2P alteration (lysine and valine at amino acid positions 986 and 987 substituted with prolines). For example, the codon-optimized S antigen sequence disclosed above (based on the Wuhan-Hu-1 reference strain (#NC_045512) and optimized for vaccinia) is further modified to encode for such an S antigen. The DNA sequence/ORF (5′ to 3′ end) is shown in FIG. 68, and the encoded protein sequence (N- to C-terminus) is shown in FIG. 69.

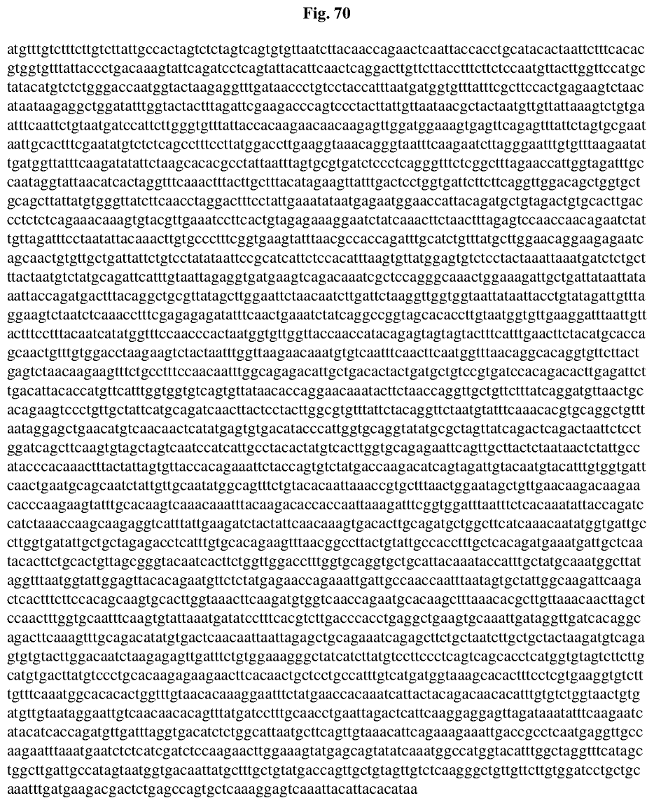

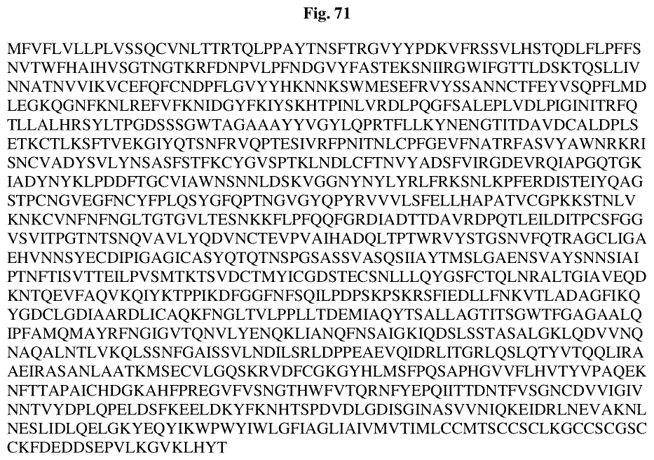

In another embodiment, the SARS-CoV-2 S antigen sequence is modified to encode for a prefusion stabilized S antigen with 2P alteration (lysine and valine at amino acid positions 986 and 987 substituted with prolines) and mutated Furin cleavage site (RRAR amino acids at positions 682-685 substituted with GSAS). For example, the codon-optimized S antigen sequence disclosed above (based on the Wuhan-Hu-1 reference strain (#NC_045512) and optimized for vaccinia) is further modified to encode for such an S antigen. The DNA sequence/ORF (5′ to 3′ end) is shown in FIG. 70, and the encoded protein sequence (N- to C-terminus) is shown in FIG. 71.

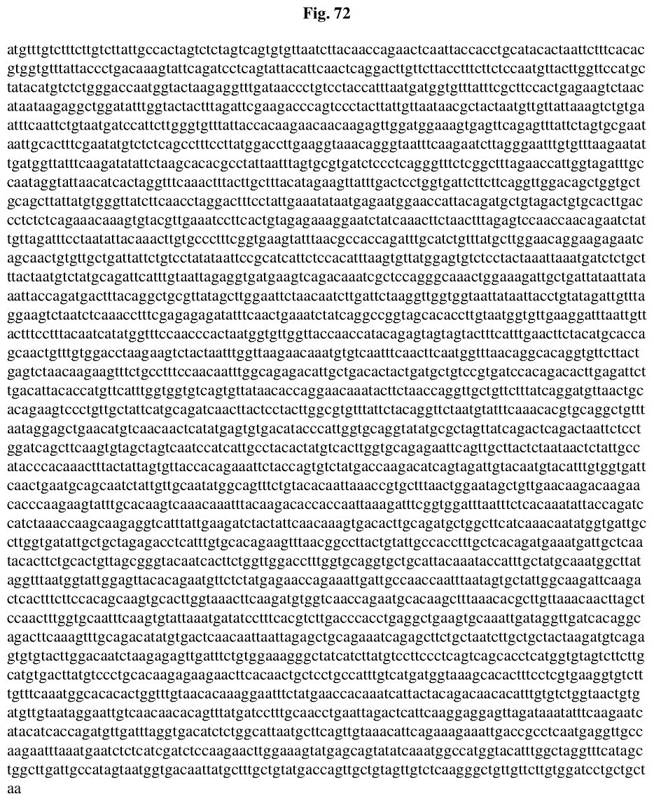

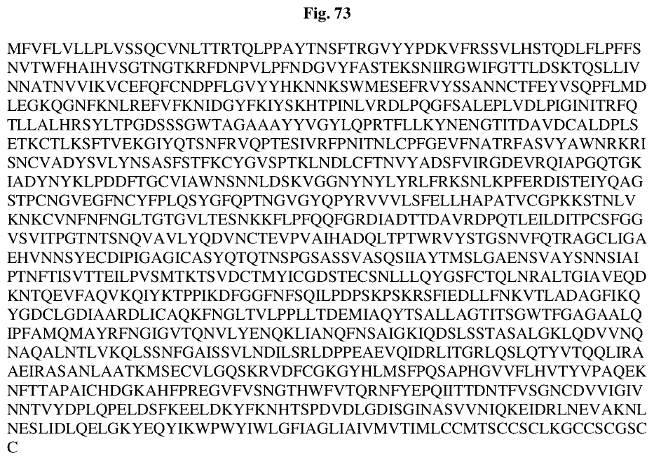

In another embodiment the SARS-CoV-2 S antigen sequence is modified to encode for a prefusion stabilized S antigen with 2P alteration (lysine and valine at amino acid positions 986 and 987 substituted with prolines), mutated Furin cleavage site (RRAR amino acids at positions 682-685 substituted with GSAS), and 19 amino acid residues at the C-terminus (KFDEDDSEPVLKGVKLHYT, SEQ ID NO: 65) deleted to prevent endoplasmic reticulum retention and to enhance cell surface expression. For example, the codon-optimized S antigen sequence disclosed above (based on the Wuhan-Hu-1 reference strain (#NC_045512) and optimized for vaccinia) is further modified to encode for such an S antigen. The DNA sequence/ORF (5′ to 3′ end) is shown in FIG. 72, and the encoded protein sequence (N- to C-terminus) is shown in FIG. 73.

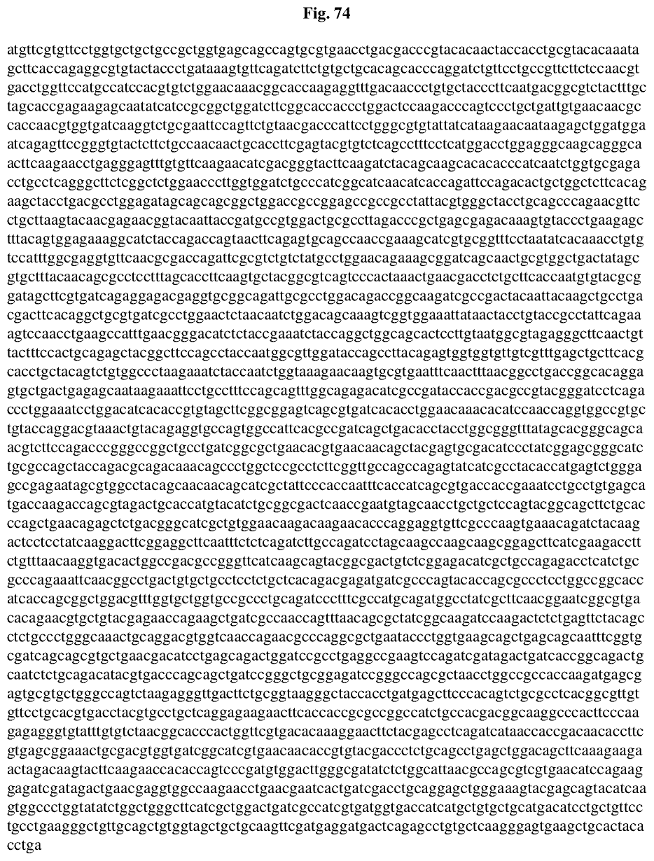

In another example the SARS-CoV-2 S antigen sequence is fully codon-optimized for human expression, additionally optimized for stability in vaccinia by silent codon alteration to avoid 4 or more of the same nucleotides in consecutive order, and encodes for an S antigen with mutated Furin cleavage site and stabilizing 2P mutation, as shown in FIG. 74.

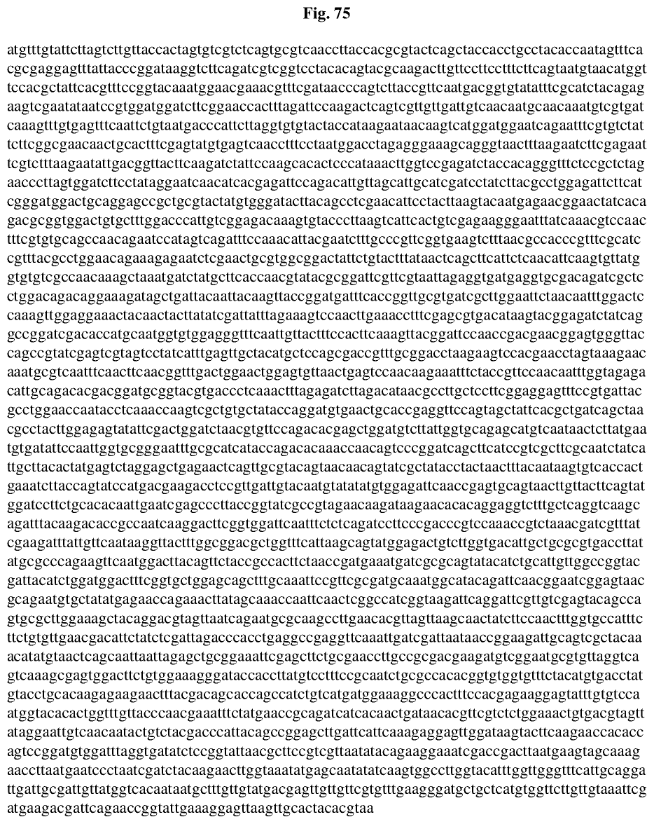

In another example the SARS-CoV-2 S antigen sequence is fully codon-optimized for Vaccinia virus expression, additionally optimized for stability in vaccinia by silent codon alteration to avoid 4 or more of the same nucleotides in consecutive order, and encodes for an S antigen with mutated Furin cleavage site and stabilizing 2P mutation, as shown in FIG. 75.

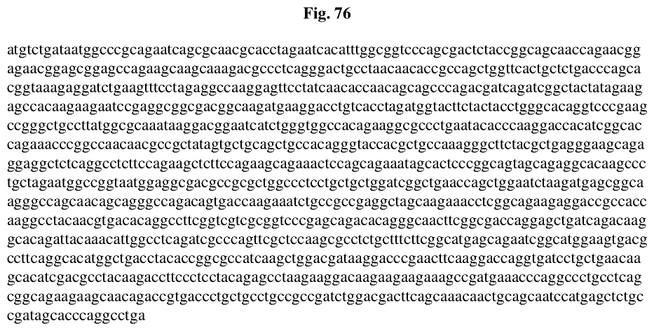

In one example the SARS-CoV-2 N antigen sequence is fully codon-optimized for human expression and additionally optimized for stability in vaccinia by silent codon alteration to avoid 4 or more of the same nucleotides in consecutive order, as shown in FIG. 76.

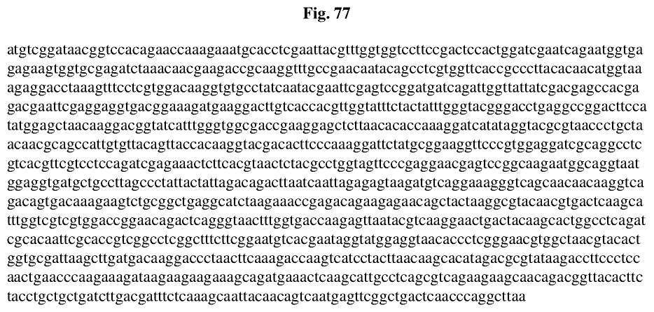

In another example the SARS-CoV-2 N antigen sequence is fully codon-optimized for Vaccinia virus expression and additionally optimized for stability in vaccinia by silent codon alteration to avoid 4 or more of the same nucleotides in consecutive order, as shown in FIG. 77.

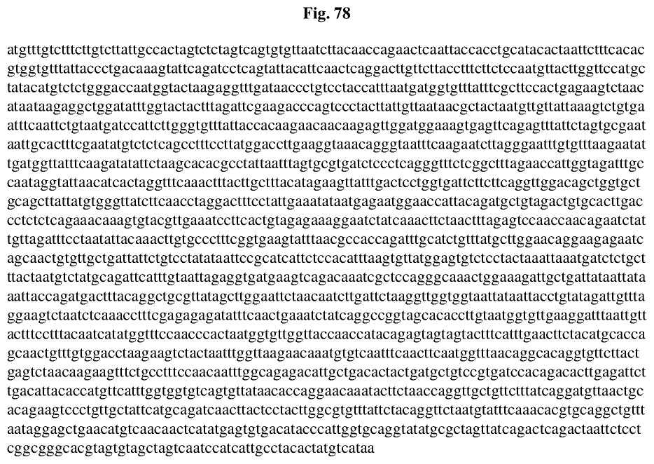

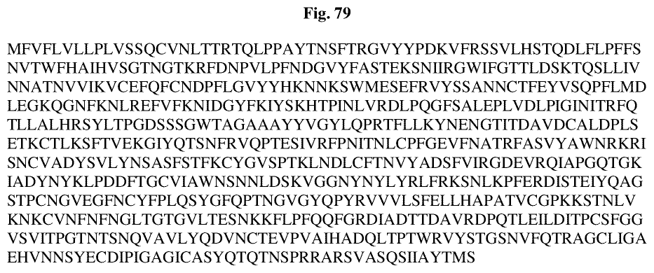

In another embodiment, the SARS-CoV-2 S antigen sequence encodes only for the S1 domain that encompasses 698, 685, or 680 amino acid residues or even shorter amino acid sequences of the N-terminus of the S protein. For example, the S antigen sequence based on the Wuhan reference strain (#NC_045512) and optimized for stability in vaccinia as disclosed above encodes for an S1 domain encompassing 698 amino acid residues of the N-terminus of the SARS-CoV-2 S antigen. The DNA sequence/ORF (5′ to 3′ end) is shown in FIG. 78, and the encoded protein sequence (N- to C-terminus) is shown in FIG. 79.

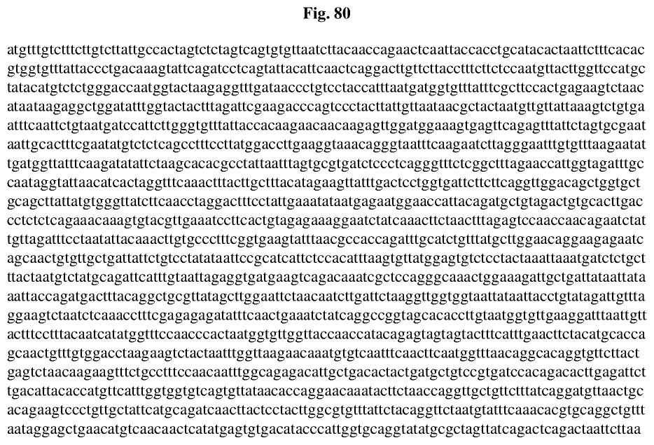

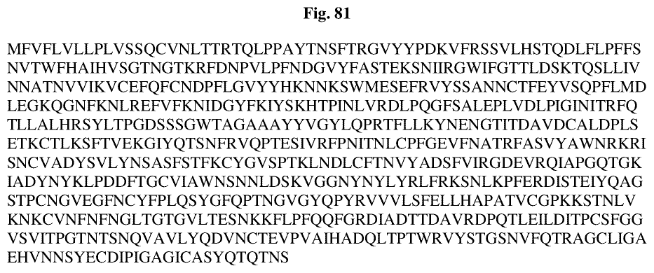

In another example the SARS-CoV-2 S antigen sequence based on the Wuhan reference strain (#NC_045512) and optimized for stability in vaccinia as disclosed above encodes for an S1 domain encompassing 680 amino acid residues of the SARS-CoV-2 S antigen. The DNA sequence/ORF (5′ to 3′ end) is shown in FIG. 80, and the encoded protein sequence (N- to C-terminus) is shown in FIG. 81.

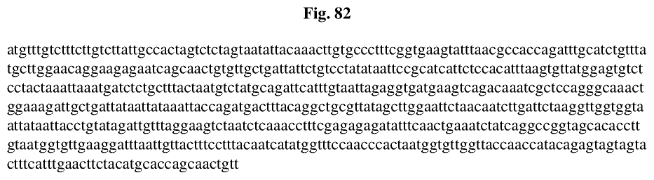

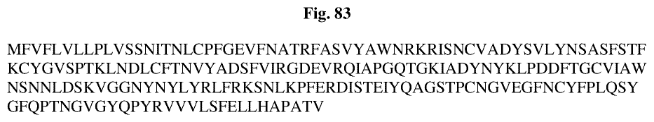

In another embodiment, the SARS-CoV-2 S antigen sequence encodes only for the receptor binding domain (RBD) that encompasses amino acid residues 331 to 524 or 319 to 541 of the S antigen, or a longer or shorter fragment thereof comprising the RBD domain. For example, the S antigen sequence based on the Wuhan reference strain (#NC_045512) and optimized for stability in vaccinia as disclosed above encodes for an RBD encompassing amino acid residues 331-524 of the SARS-CoV-2 S antigen fused to the signal peptide of the S antigen (C-terminal 13 amino acids comprising MFVFLVLLPLVSS, SEQ ID NO: 41). The DNA sequence/ORF (5′ to 3′ end) is shown in FIG. 82, and the encoded protein sequence (N- to C-terminus) is shown in FIG. 83.

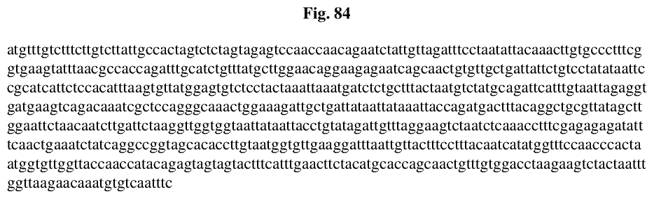

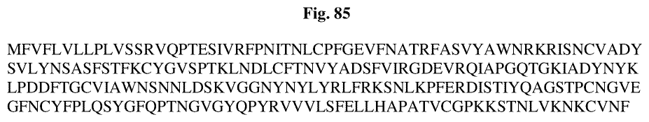

In another embodiment, the SARS-CoV-2 S antigen sequence is based on the Wuhan reference strain (#NC_045512) and optimized for stability in vaccinia as disclosed above encodes for an RBD encompassing amino acid residues 319-541 of the SARS-CoV-2 S antigen fused to the signal peptide of the S antigen (C-terminal 13 amino acids comprising MFVFLVLLPLVSS, SEQ ID NO: 41). The DNA sequence/ORF (5′ to 3′ end) is shown in FIG. 84, and the encoded protein sequence (N- to C-terminus) is shown in FIG. 85.

In another embodiment the SARS-CoV-2 S antigen sequence encodes for an S antigen that contains one or more mutations or alterations at any amino acid position of the S antigen. These mutations or alterations may include amino acid substitutions, insertion, or deletions. The mutations may be involved in immune evasion that renders SARS-CoV-2 resistant to certain humoral and cellular immune responses, including neutralizing antibodies (NAb). The mutations may include one or more alterations in the RBD domain (amino acid residues 319-541) that mediates binding to and entry into host cells and that is the primary target of NAb. For example, the RBD mutations may include an asparagine to tyrosine substitution at amino acid position 501 of the S antigen (N501Y); a glutamic acid to lysine substitution at amino acid position 484 of the S antigen (E484K); a glutamic acid to glutamine substitution at amino acid position 484 of the S antigen (E484Q); a lysine to asparagine substitution at amino acid position 417 of the S antigen (K417N); a lysine to threonine substitution at amino acid position 417 of the S antigen (K417T); a leucine to arginine substitution at amino acid position 452 of the S antigen (L452R); a serine to asparagine substitution at amino acid position 477 of the S antigen (S477N); an asparagine to lysine substitution at amino acid position 439 of the S antigen (N439K); a serine to proline substitution at amino acid position 494 of the S antigen (S494P); an alanine to serine substitution at amino acid position 520 of the S antigen (S520S); a tyrosine to phenylalanine substitution at amino acid position 453 of the S antigen (Y453F).

In another embodiment the SARS-CoV-2 S antigen sequence encodes for an S antigen that contains or includes a dominant mutation occurring in SARS-CoV-2 and many of its emerging variants, which is the D614G mutation (aspartic acid to glycine substitution at amino acid position 614 of the S antigen).

In another embodiment the SARS-CoV-2 antigen sequence encodes for an S antigen that includes all mutations, a subset of the mutations, or a combination of the mutations occurring in the emerging SARS-CoV-2 variants that are of particular concern (variants of concern, or VOC), such as the B.1.351 variant lineage first identified in South Africa, the B.1.1.7 variant lineage first identified in the United Kingdom (UK), the P.1 variant lineage first identified in Brazil, the B.1.429+B.1.427 variant lineage identified in California, or the B.1.617 variant lineage first identified in India. Modified antigen sequences with mutations based on other SARS-CoV-2 variant lineages described by the PANGO tool (cov-lineages.org) or under GISAID (gisaid.org) may also be used. For example, the SARS-CoV-2 S antigen sequence may encode for an S antigen that contains mutations of the B.1.351 variant lineage identified in South Africa variant, including N501Y, E484K, and K417N substitutions in the RBD domain, the D614G mutation, a leucine to phenylalanine substitution at amino acid position 18 of the S antigen (L18F), an aspartic acid to alanine substitution at amino acid position 80 of the S antigen (D80A), an aspartic acid to glycine substitution at amino acid position 215 of the S antigen (D215G), a deletion of three amino acids at position 242-244 (leucine, alanine, leucine) of the S antigen (Del242-244), an arginine to isoleucine substitution at amino acid position 246 of the S antigen (R246I ), and an alanine to valine substitution at amino acid position 701 of the S antigen (A701V).

In another embodiment, the SARS-CoV-2 S antigen sequence encodes for an S antigen that includes mutations of the B.1.1.7 variant lineage identified in the UK, including N501Y in the RBD, D614G, a deletion of two amino acids at positions 69 and 70 (histidine, valine) of the S antigen (Del69/70), a deletion of the tyrosine residue at position 144 of the S antigen (Del144), an alanine to aspartic acid substitution at amino acid position 570 of the S antigen (A570D), a proline to histidine substitution at amino acid position 681 of the S antigen (P681H), a threonine to isoleucine substitution at amino acid position 716 of the S antigen (T716I), a serine to alanine substitution at amino acid position 982 of the S antigen (S982A), and an aspartic acid to histidine substitution at amino acid position 1118 of the S antigen (D1118H). The encoded S antigen based on the UK variant may additionally include E484K and K417N or K417T or other mutations in the RBD domain as disclosed above.

In another embodiment the SARS-CoV-2 S antigen sequence encodes for an S antigen that includes the mutations of the P.1 variant lineage identified in Brazil, including D614G, N501Y, E484K, K417T, L18F, a threonine to asparagine substitution at amino acid position 20 of the S antigen (T20N), a proline to serine substitution at amino acid position 26 of the S antigen (P26S), an aspartic acid to tyrosine substitution at amino acid position 138 of the S antigen (D138Y), an arginine to serine substitution at amino acid position 190 of the S antigen (R190S), a histidine to tyrosine substitution at amino acid position 655 of the S antigen (H655Y), a threonine to isoleucine substitution at amino acid position 1027 of the S antigen (T1027I), and a valine to phenylalanine substitution at amino acid position 1176 of the S antigen (V1176F). The encoded S antigen may additionally include other mutations in the RBD domain as disclosed above, such as L452R or Y453F.

In another embodiment, the SARS-CoV-2 S antigen sequence encodes for an S antigen that includes the mutations of B.1.429+B.1.427 variant lineage identified in California, including D614G, L452R in the RBD, a serine to isoleucine substitution at amino acid position 13 of the S antigen (S13I), and a tryptophan to cysteine mutation at amino acid position 152 of the S antigen (W152C). The encoded S antigen based on the Southern California variant may additionally include N501Y, E484K, E484Q, or other RBD mutations.

In another embodiment, the SARS-CoV-2 antigen sequence encodes for an S antigen that contains different combinations of the mutations that occur in the VOC. For example, the S antigen sequence may encode for an S antigen that combines the mutations or only a subset of the mutations that occur in the B.1.429+B.1.427 and B.1.1.7 variant lineages, the B.1.429+B.1.427 and B.1.351 variant lineages, the B.1.429+B.1.427 and P.1 variant lineages, the B.1.1.7 and B.1.351 lineages, the B.1.1.7 and P.1 lineages, the B.1.351 and P.1 lineages, or other combinations of these lineages. These combinations of mutations may additionally include any of the RBD mutations as disclosed above such as N501Y, E484K, K417N, K417T, L452R, S477N, N439K, S520S, and Y453F.

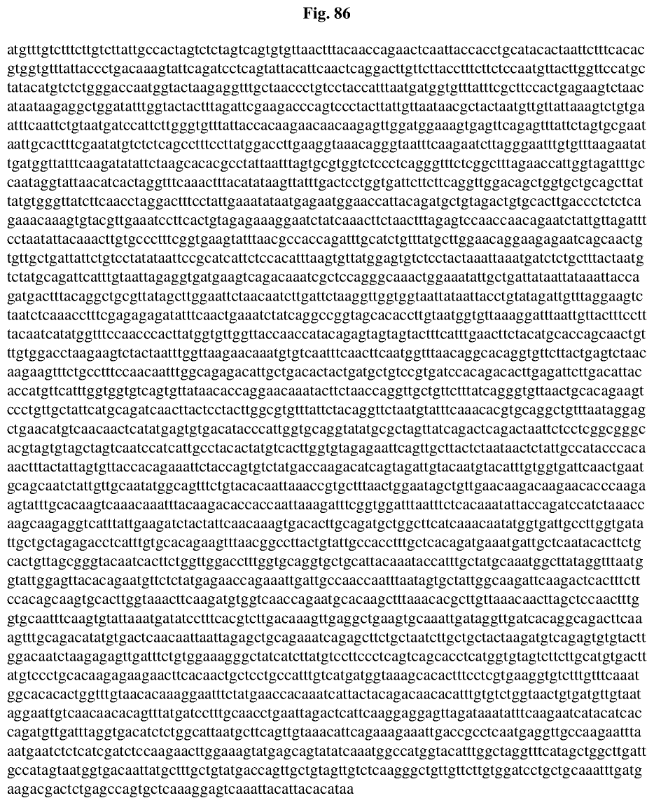

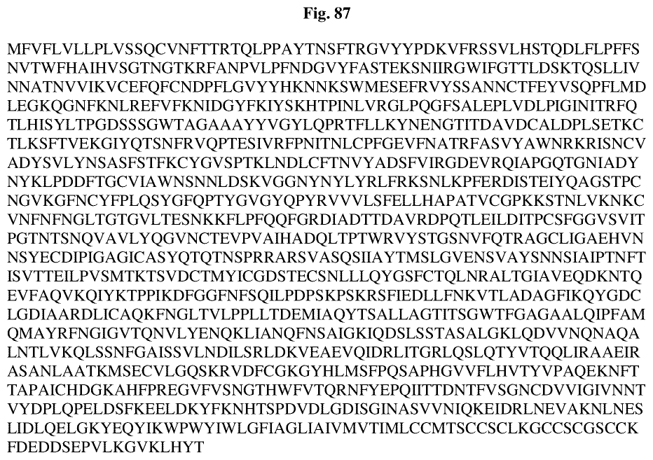

In another embodiment, the codon-optimized SARS-CoV-2 S antigen sequence disclosed above (based on the Wuhan-Hu-1 reference strain (#NC_045512) and optimized for vaccinia) is further modified to encode for an S antigen that includes mutations of the B.1.351 variant lineage identified in South Africa (N501Y, E484K, K417N, L18F, D80A, D215G, Del242-244, R246I, D614G, A701V). The DNA sequence/ORF (5′ to 3′ end) is shown in FIG. 86, and the encoded protein sequence (N- to C-terminus) is shown in FIG. 87.

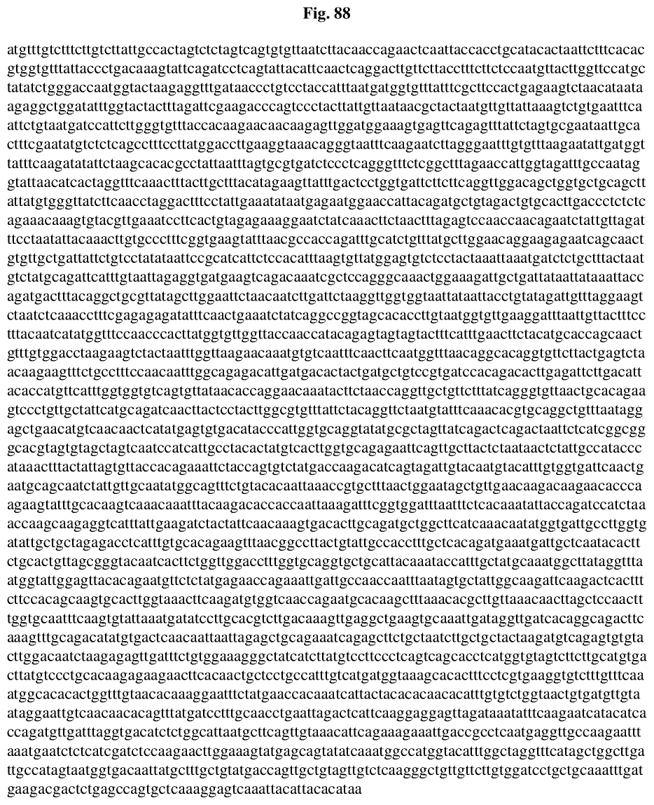

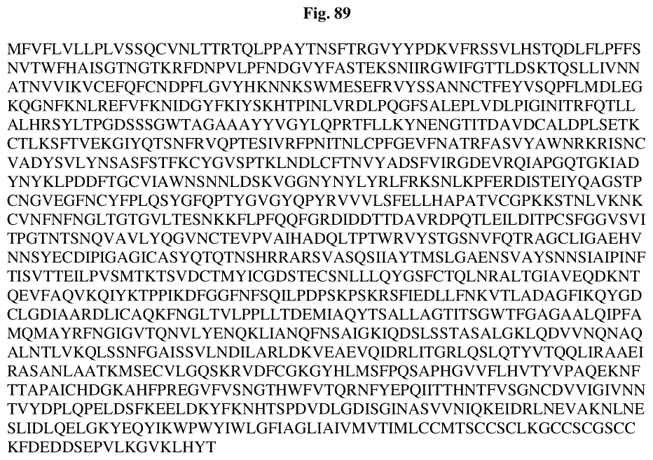

In another example the codon-optimized SARS-CoV-2 S antigen sequence disclosed above (based on the Wuhan-Hu-1 reference strain (#NC_045512) and optimized for vaccinia) is further modified to encode for an S antigen that includes mutations of the B.1.1.7 variant lineage identified in the UK (N501Y, Del69/70, Del144, A570D, D614G, P681H, T716I, S982A, D1118H). The DNA sequence/ORF (5′ to 3′ end) is shown in FIG. 88, and the encoded protein sequence (N- to C-terminus) is shown in FIG. 89.

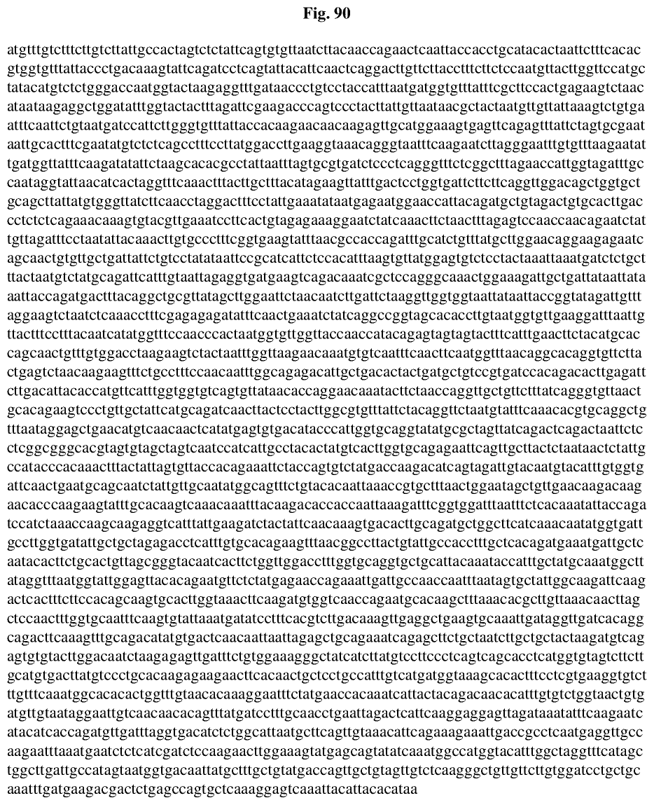

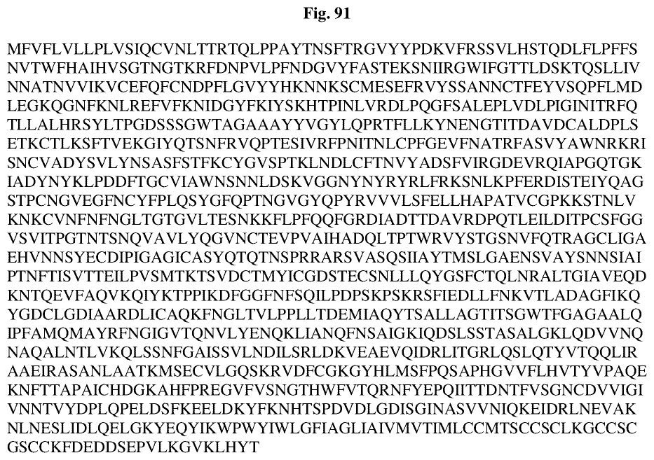

In another example the codon-optimized SARS-CoV-2 S antigen sequence disclosed above (based on the Wuhan-Hu-1 reference strain (#NC_045512) and optimized for vaccinia) is further modified to encode for an S antigen that includes mutations of the B.1.429+B.1.427 variant lineage identified in California (D614G, L452R, S13I, W152C). The DNA sequence/ORF (5′ to 3′ end) is shown in FIG. 90, and the encoded protein sequence (N- to C-terminus) is shown in FIG. 91.

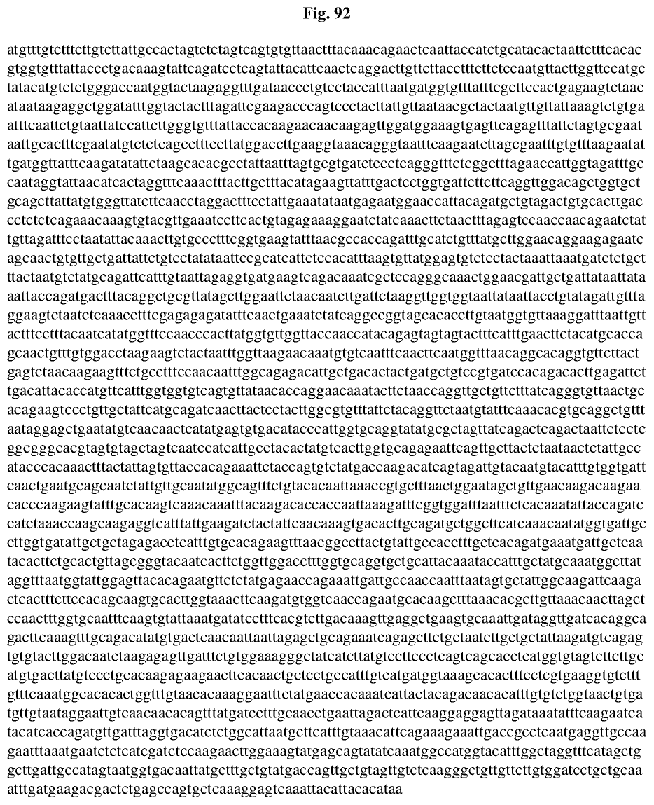

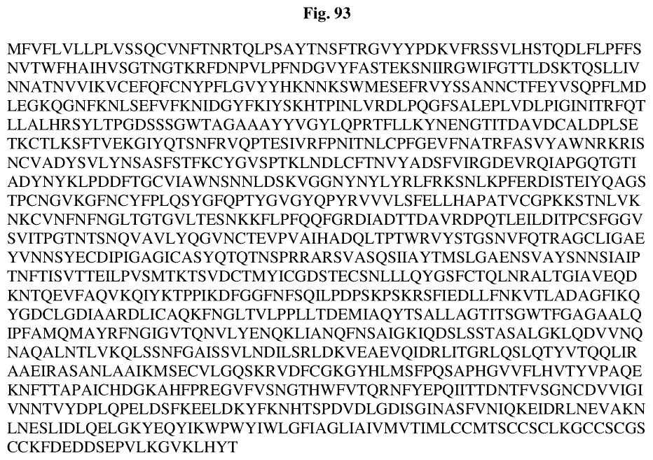

In another example the codon-optimized SARS-CoV-2 S antigen sequence disclosed above (based on the Wuhan-Hu-1 reference strain (#NC_045512) and optimized for vaccinia) is further modified to encode for an S antigen that includes mutations of P.1 variant lineage identified in Brazil (N501Y, E484K, K417T, L18F, T20N, P26S, D138Y, R190S, H655Y, T1027I, V1176F). The DNA sequence/ORF (5′ to 3′ end) is shown in FIG. 92, and the encoded protein sequence (N- to C-terminus) is shown in FIG. 93.

In another embodiment the SARS-CoV-2 S antigen sequence is further modified to encode for an S antigen based on the B.1.429+B.1.427, B.1.1.7, B.1.351, or P.1 variant lineages that includes additionally 2P stabilizing mutations (lysine 986 and valine 987 substituted with prolines (K986P and V987P), a mutated Furin cleavage site (682-685 RRAR to GSAS), and/or C-terminal 19 amino acid residues (KFDEDDSEPVLKGVKLHYT, SEQ ID NO: 65) deleted.

In another embodiment different SARS-CoV-2 antigen sequences with different codon usage are utilized to encode for different S antigens based on the original Wuhan-Hu-1 reference strain and the B.1.429+B.1.427, B.1.1.7, B.1.351, or P.1 variant lineages. These antigen sequences can be inserted together into one sMVA vector or into separate sMVA vectors using different insertion sites (e.g. Del2, Del3, IGR69/70). For example, the following three antigen sequences maybe used to co-express the S antigens of the Wuhan-Hu-1 reference strain, the South African variant B.1.351, and the UK variant B.1.1.7.

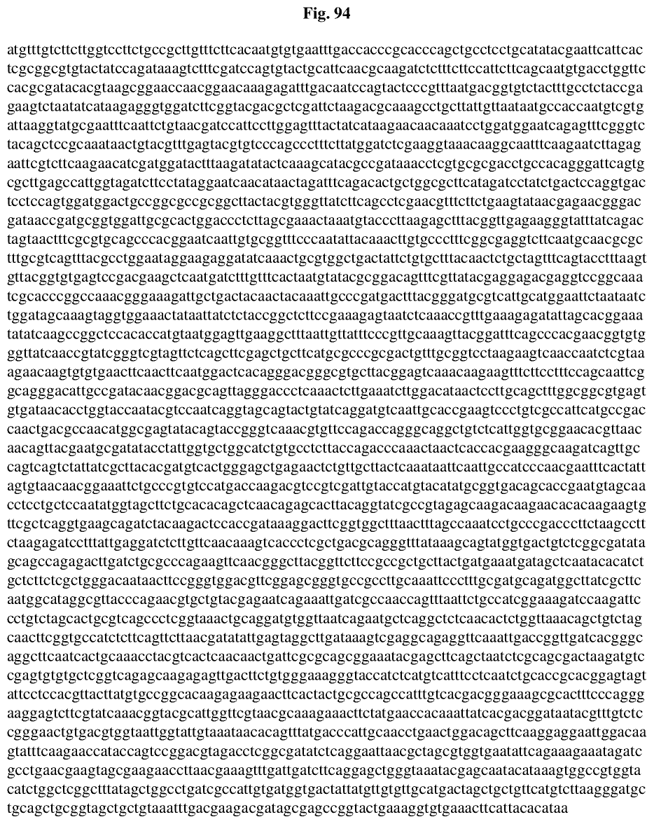

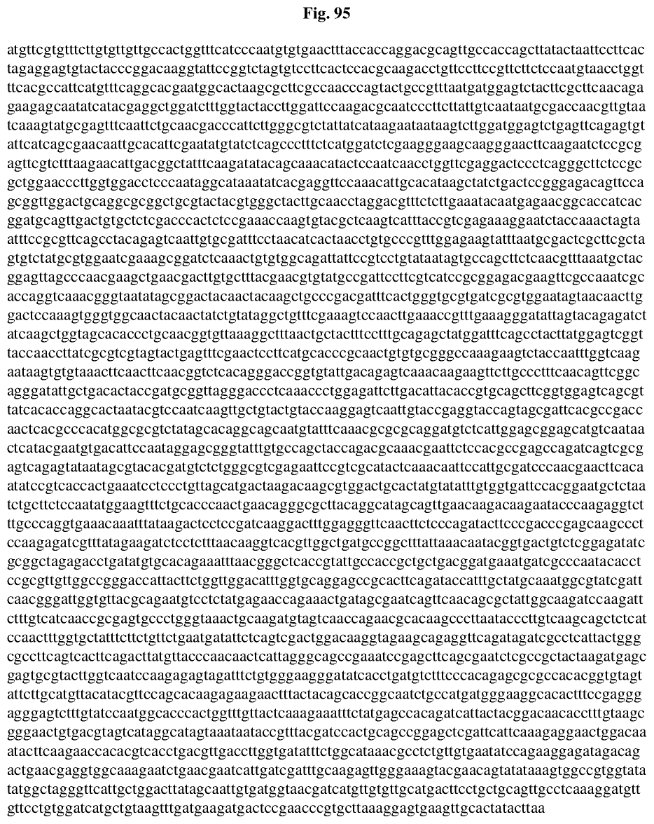

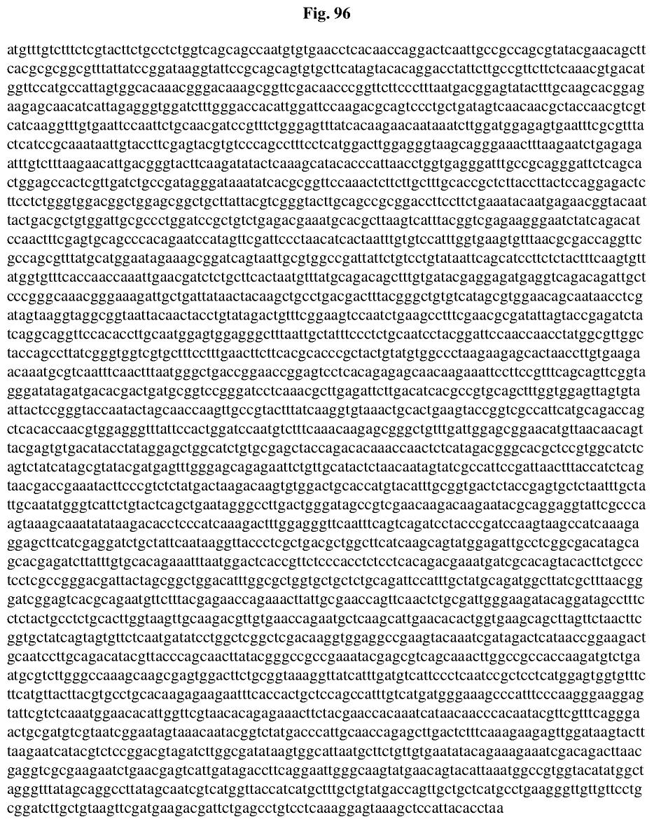

The sequence encoding the S antigen of the Wuhan-Hu-1 reference strain is shown in FIG. 94, the sequence encoding the S antigen of the South African variant B.1.351 is shown in FIG. 95, and the sequence encoding the S antigen of the UK variant B.1.1.7 is shown in FIG. 96.

In another embodiment multiple different SARS-CoV-2 RBD domains (amino acid residues 319-541) based on the original Wuhan-Hu-1 reference strain, or the B.1.429+B.1.427, B.1.1.7, B.1.351, P.1, or B.1.617 variants, or other emerging SARS-CoV-2 variants can be co-expressed either from one vector or from separate vectors though the utilization of different codon usage. These RBD domains can be co-expressed each by its one Vaccinia promoter (e.g., mH5) or co-expressed through polycistronic expression constructs in which the individual RBD domains are connected through different linkers sequences (e.g., GS linkers) or 2A peptides of picornaviruses mediating ribosomal skipping. In addition, one or more of these domains can be fused at the N-terminus to the SARS-CoV-2 signal sequence (first 13 or 16 N-terminal amino acids of the S protein), at the N- or C-terminus to the T4 fibritin Foldon domain (GYIPEAPRDGQAYVRKDGEWVLLSTFL, SEQ ID NO: 45) that mediates trimerization, and/or at the C-terminus fused to the transmembrane domain (TM) and cytoplasmic domain (CT) of the SARS-CoV-2 S protein, wherein the last 19 amino acids of the CT domain may be deleted to avoid ER retention and enhance cell surface expression.

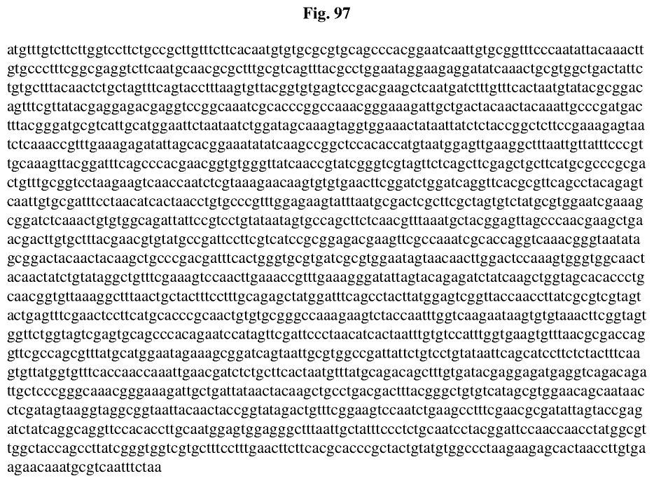

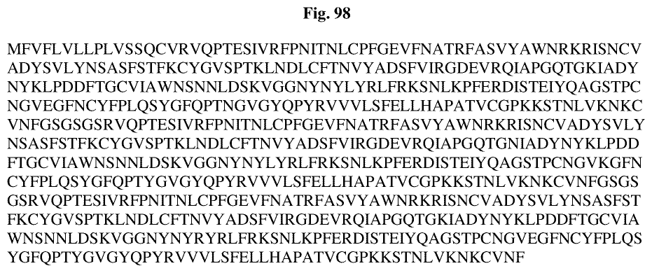

For example, the RBD domain of the Wuhan-Hu-1 reference strain, the RBD domain of the B.1.351 variant, and an RBD domain combining N501Y and L452R mutations of the B.1.1.7 and B.1.429+B.1.427 variants can be co-expressed through a triple polycistronic expression construct which comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42). The DNA sequence/ORF (5′ to 3′ end) is shown in FIG. 97, and the encoded protein sequence (N-to C-terminus) is shown in FIG. 98.

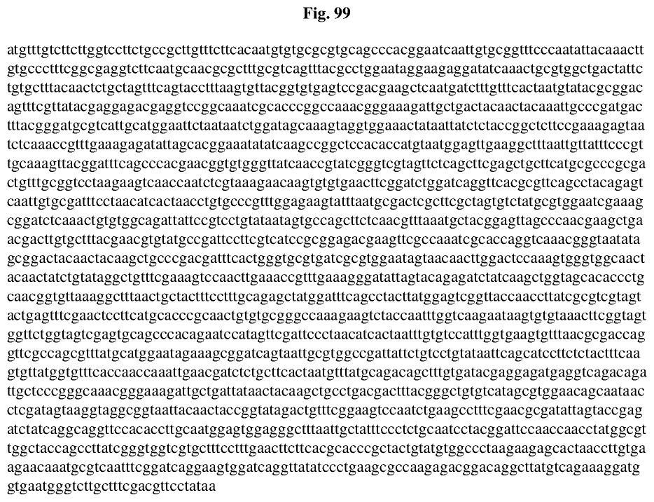

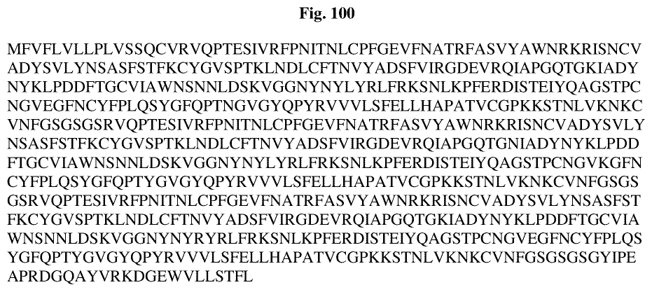

In another example, the RBD domain of the Wuhan-Hu-1 reference strain, the RBD domain of the B.1.351 variant, and a RBD domain combining the N501Y and L452R mutations of the B.1.1.7 and B.1.429+B.1.427 variants can be co-expressed through a triple polycistronic expression construct comprising at the N-terminus a signal peptide of the S antigen (MFVFLVLLPLVSSQCV, SEQ ID NO: 41 and at the C-terminus a T4 Foldon domain (GYIPEAPRDGQAYVRKDGEWVLLSTFL, SEQ ID NO: 45) and in which the RBD domains are connected through GS linkers such as GSGSGS (SEQ ID NO: 42). The DNA sequence/ORF (5′ to 3′ end) is shown in FIG. 99, and the encoded protein sequence (N-to C-terminus) is shown in FIG. 100.

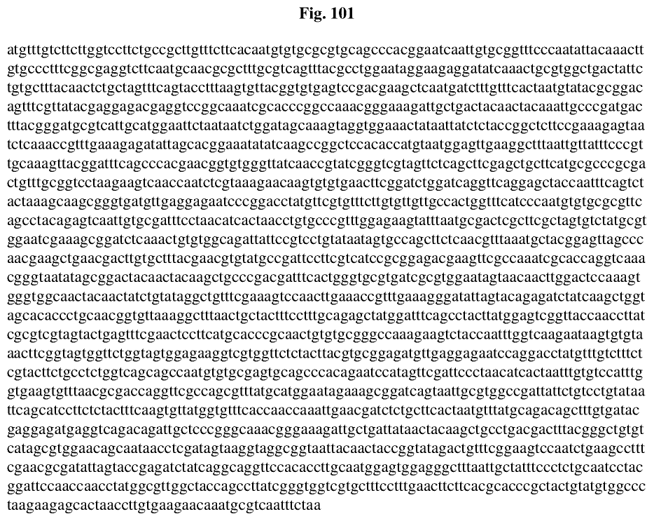

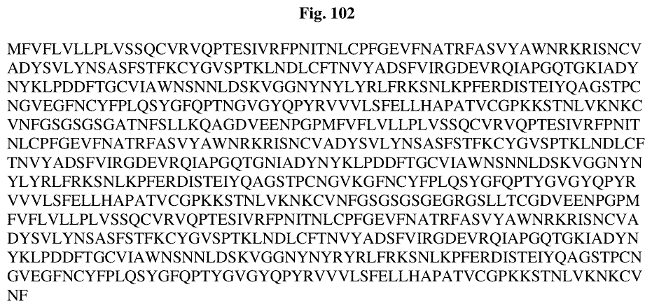

In another example, the RBD domain of the Wuhan-Hu-1 reference strain, the RBD domain of the B.1.351 variant, and an RBD domain combining N501Y and L452R mutations of the B.1.1.7 and B.1.429+B.1.427 variants can be co-expressed through a polycistronic expression construct in which each RBD domain comprises at the N-terminus a signal peptide of the S antigen (MFVFLVLLPLVSSQCV, SEQ ID NO: 41) and in which the RBD domains are connected through GS linkers such as GSGSGS (SEQ ID NO: 42) and P2A and T2A peptides. The DNA sequence/ORF (5′ to 3′ end) is shown in FIG. 101, and the encoded protein sequence (N- to C-terminus) is shown in FIG. 102.

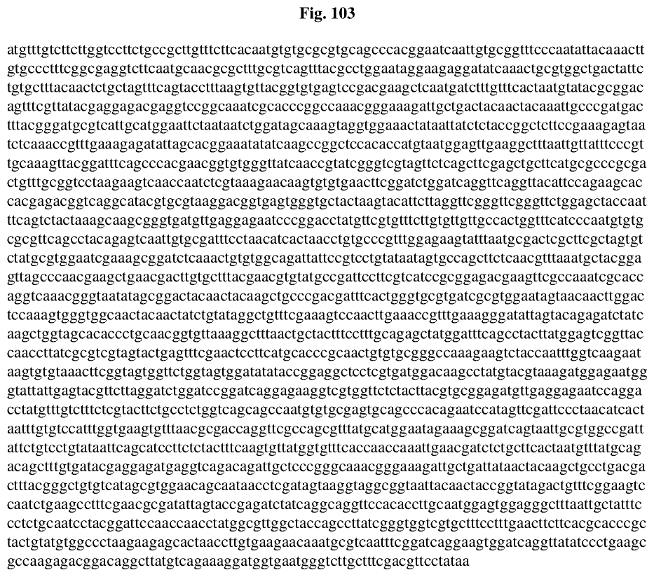

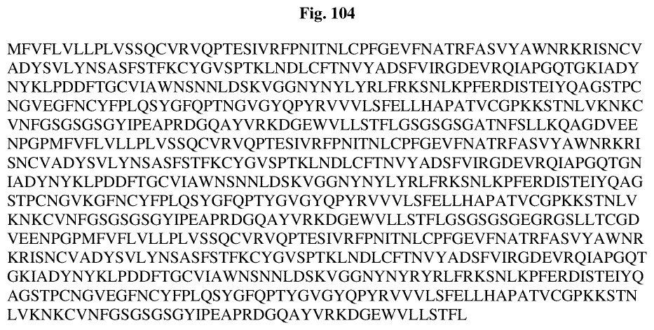

In another example, the RBD domain of the Wuhan-Hu-1 reference strain, the RBD domain of the B.1.351 variant, and a RBD domain combining N501Y and L452R mutations of the B.1.1.7 and B.1.429+B.1.427 variants can be co-expressed through a polycistronic expression construct in which the RBD domains are connected through GS linkers (GSGSGS, SEQ ID NO: 42) and P2A and T2A peptides and in which each RBD domain is fused at the N-terminus to a S signal peptide (MFVFLVLLPLVSSQCV, SEQ ID NO: 41) and at the C-terminus to a T4 Foldon domain (GYIPEAPRDGQAYVRKDGEWVLLSTFL, SEQ ID NO: 45). The DNA sequence/ORF (5′ to 3′ end) is shown in FIG. 103, and the encoded protein sequence (N- to C-terminus) is shown in FIG. 104.

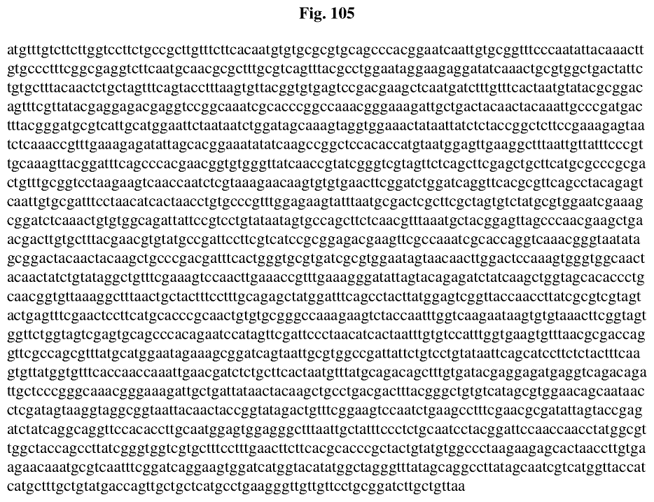

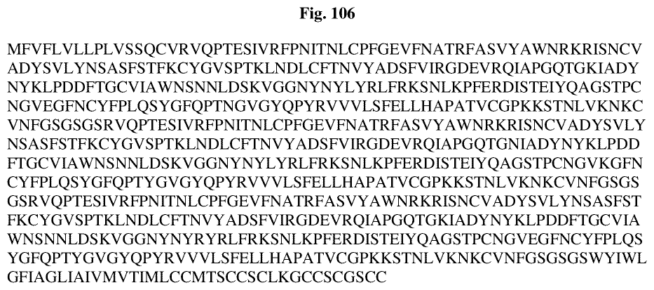

In another example, the RBD domain of the Wuhan-Hu-1 reference strain, the RBD domain of the B.1.351 variant, and an RBD domain combining N501Y and L452R mutations of the B.1.1.7 and B.1.429+B.1.427 variants can be co-expressed through a polycistronic expression construct comprising at the N-terminus a signal peptide of the S antigen (MFVFLVLLPLVSSQCV, SEQ ID NO: 41) and at the C-terminus TM and CT domains (without the last 19 amino acids) of the S antigen and in which the RBD domains are connected through GS linkers such as GSGSGS (SEQ ID NO: 42). The DNA sequence/ORF (5′ to 3′ end) is shown in FIG. 105, and the encoded protein sequence (N-to C-terminus) is shown in FIG. 106.

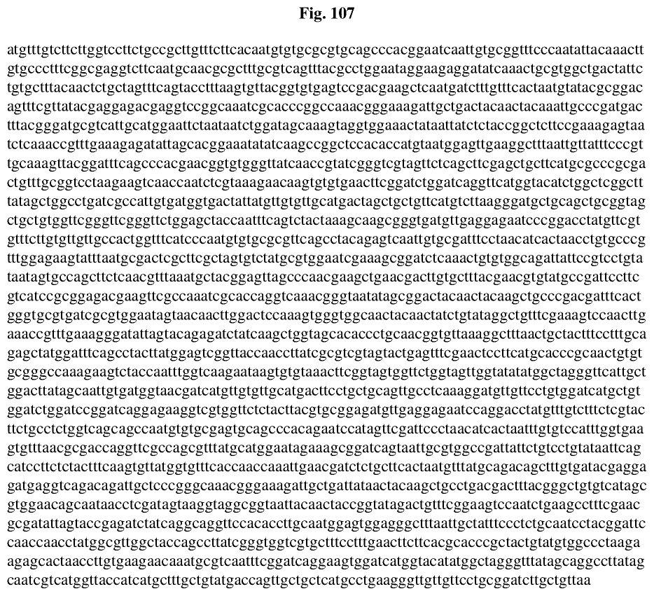

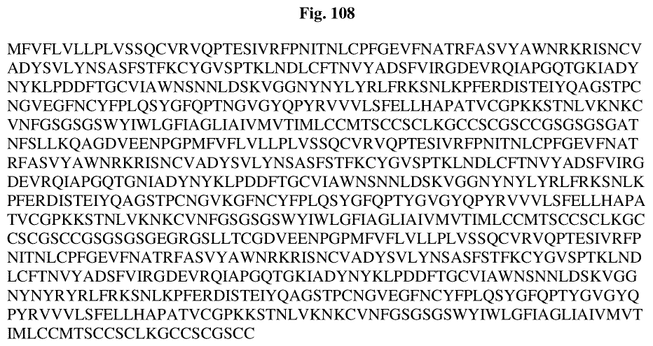

In another example, the RBD domain of the Wuhan-Hu-1 reference strain, the RBD domain of the B.1.351 VOC, and an RBD domain combining N501Y and L452R mutations of the B.1.1.7 and B.1.429+B.1.427 VOC can be co-expressed through a polycistronic expression construct in which each of the RBD domains comprises at the N-terminus a signal peptide of the S protein (MFVFLVLLPLVSSQCV, SEQ ID NO: 41) and at the C-terminus with TM domain and CT domain (without the last 19 amino acids KFDEDDSEPVLKGVKLHYT, SEQ ID NO: 65) of the S antigen and in which the RBD domains are connected through GS linkers such as GSGSGS (SEQ ID NO: 42) and P2A and T2A peptide sequences. The DNA sequence/ORF (5′ to 3′ end) is shown in FIG. 107, and the encoded protein sequence (N- to C-terminus) is shown in FIG. 108.

In another embodiment, the SARS-CoV-2 N antigen sequence encodes for an N antigen which comprises one or more mutations at different amino acid positions of the N protein. These mutations or alterations may include amino acid substitutions, insertions, or deletions. The mutations maybe based on the N-specific mutations that occur in the South African VOC B.1.351, the California variant B.1.429+B.1.427, the UK variant B.1.1.7, the Brazilian variant P.1, the Indian variant B.1.617, or any other emerging SARS-CoV-2 VOC.

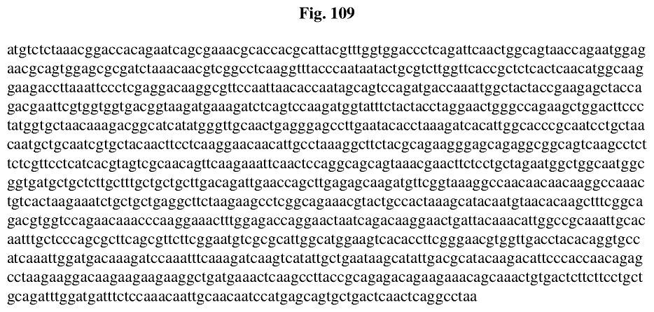

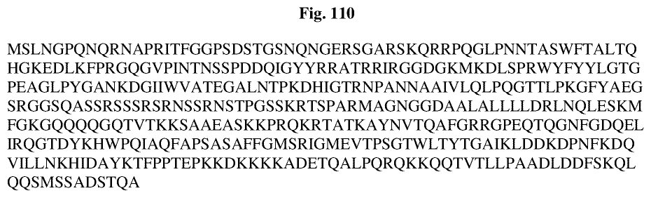

For example, the SARS-CoV-2 N antigen sequence encodes for an N antigen that includes the N-specific mutations that occur in the B.1.1.7 variant lineage identified in the UK. These mutations include an aspartic acid to leucine substitution at amino acid position 3 of the N protein (D3L), a serine to phenylalanine substitution at amino acid position 235 of the N protein (S235F), an arginine to lysine substitution at amino acid position 203 of the N protein (R203K), and a glycine to arginine substitution at amino acid position 204 of the N protein (G204R). The DNA sequence/ORF (5′ to 3′ end) is shown in FIG. 109, and the encoded protein sequence (N- to C-terminus) is shown in FIG. 110.

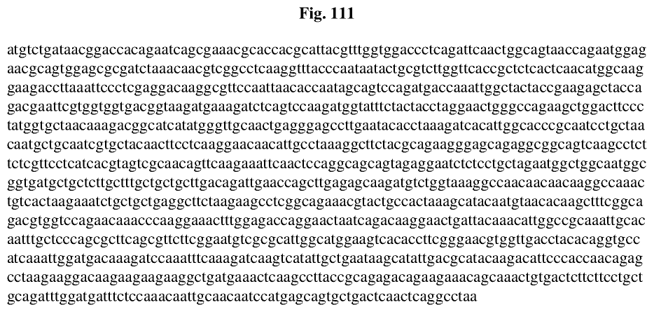

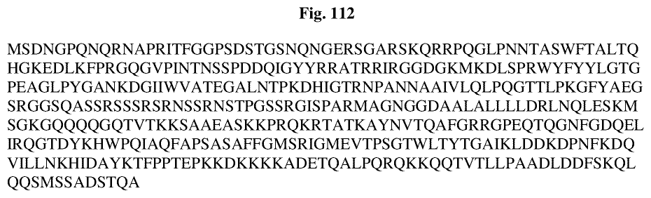

In another example, the codon-optimized SARS-CoV-2 N antigen sequence encodes for an N antigen that includes the N-specific mutations that occur in the B.1.351 variant lineage identified in South Africa. This includes a threonine to isoleucine substitution at amino acid position 205 of the N protein (T205I). The DNA sequence/ORF (5′ to 3′ end) is shown in FIG. 111, and the encoded protein sequence (N- to C-terminus) is shown in FIG. 112.

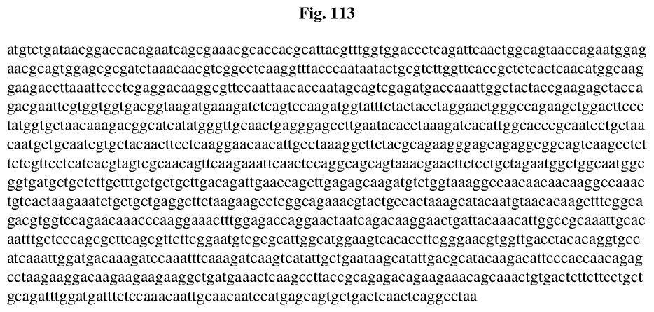

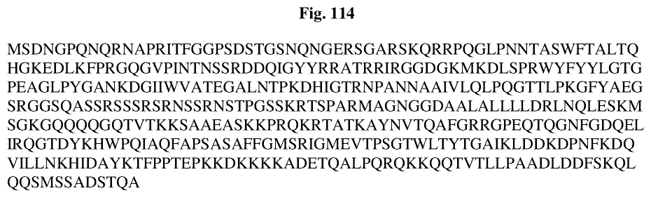

In another example, the codon-optimized SARS-CoV-2 N antigen sequence encodes for an N antigen that includes the N-specific mutations that occur in the P.1 variant lineage identified in Brazil. This includes a proline to arginine substitution at amino acid position 80 of the N protein (P80R), as well as R203K and G204R. The DNA sequence/ORF (5′ to 3′ end) is shown in FIG. 113, and the encoded protein sequence (N- to C-terminus) is shown in FIG. 114.

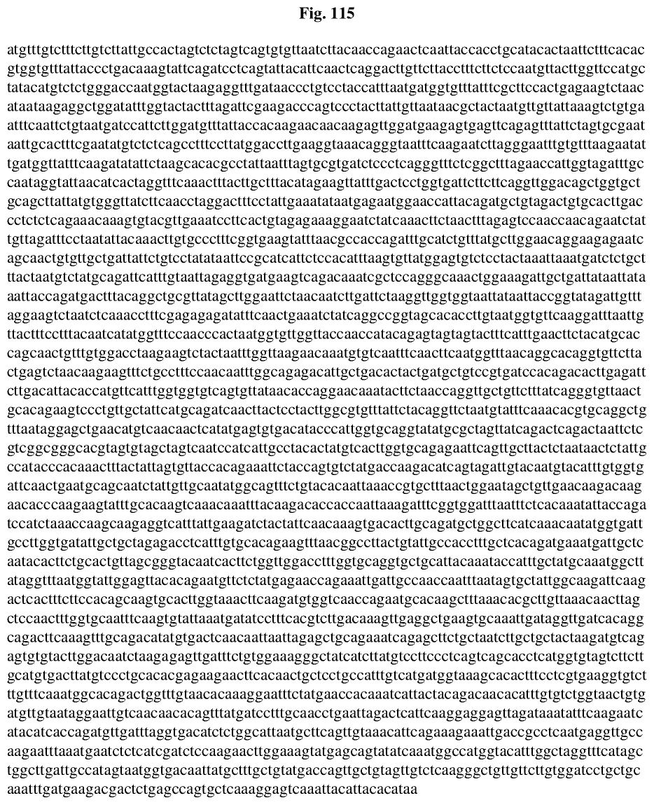

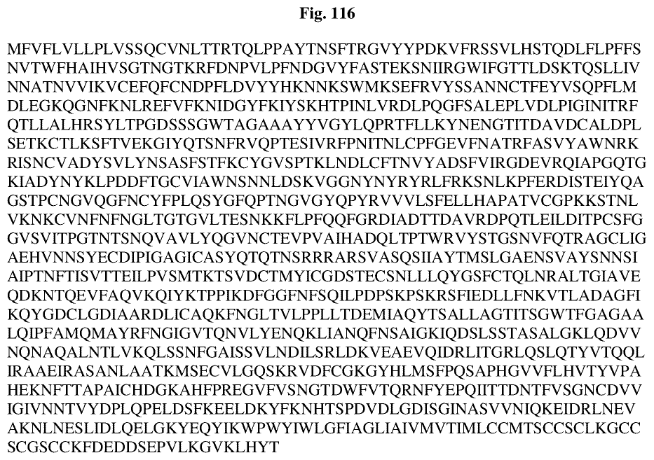

In another embodiment, the codon-optimized SARS-CoV-2 S antigen sequence disclosed above (based on the Wuhan-Hu-1 reference strain (#NC_045512) and optimized for vaccinia) is further modified to encode for an S antigen that includes mutations of the B.1.617 variant lineage identified in India. This may include L452R and E484Q mutations in the RBD domain, D614G, a glycine to aspartic acid substitution at amino acid position 142 of the S protein (G142D), a glutamic acid to lysine substitution at amino acid position 154 of the S protein (E154K), a proline to arginine substitution at amino acid position 681 of the S protein (P681R), a glutamine to histidine substitution at amino acid position 1071 of the S protein (Q1071H), and a histidine aspartic acid substitution at amino acid position 1101 of the S protein (H1101D). The DNA sequence/ORF (5′ to 3′ end) is shown in FIG. 115, and the encoded protein sequence (N- to C-terminus) is shown in FIG. 116.

In another embodiment, the codon-optimized SARS-CoV-2 S antigen sequence disclosed above (based on the Wuhan-Hu-1 reference strain (#NC_045512) and optimized for vaccinia) is further modified to encode for an S antigen that includes mutations or a subset of the mutations of the B.1.617 variant lineage, including L452R, E484Q, D614G, G142D, E154K, P681R, Q1071H, and H1101D, a threonine to lysine substitution at amino acid position 478 (T478K), a threonine to isoleucine substitution at amino acid position 95 (T95I ), a threonine to arginine substitution at amino acid position 19 (T19R), a lysine to threonine substitution at amino acid position 77 (K77T), a aspartic acid to asparagine substitution at amino acid 950 (D950N), an arginine to threonine substitution at amino acid position 21 (R21T), a glutamine to histidine substitution at amino acid position 218 (Q218H), a deletion of glutamic acid and phenylalanine at amino acid position 156 and 157 (Del156-157), and an arginine to glycine substitution at amino acid position 158 (R158G).

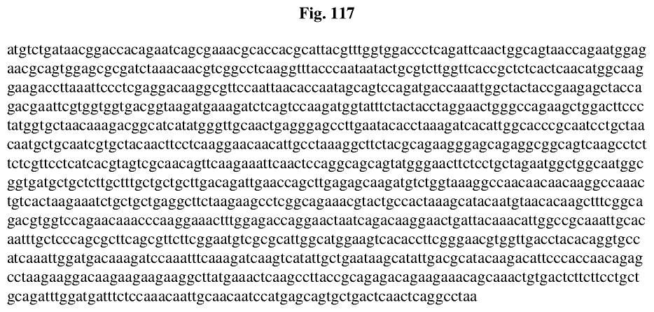

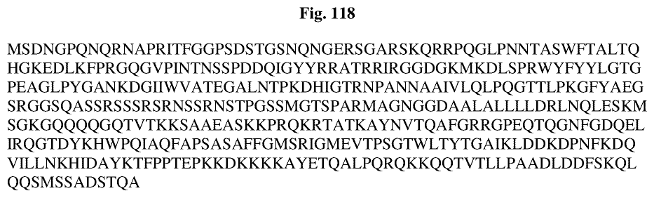

In another embodiment, the codon-optimized SARS-CoV-2 N antigen sequence encodes for an N antigen that includes the N-specific mutations that occur in the B.1.617 variant lineage identified in India. This may include an arginine to methionine substitution at amino acid position 203 of the N protein (R203M) and an aspartic acid to tyrosine substitution at amino acid position 377 of the N protein (D377Y). The DNA sequence/ORF (5′ to 3′ end) is shown in FIG. 117, and the encoded protein sequence (N- to C-terminus) is shown in FIG. 118.

In certain embodiments, the DNA sequences of two or more antigens, subunits or fragments thereof may be inserted into a single MVA insertion site or into two or more MVA insertion sites, which may be located on the same sMVA fragment or on different sMVA fragments. For example, the DNA sequences of two or more antigens, subunits or fragments thereof can be inserted in two different MVA insertion sites, both located on sMVA F1. In another example, the DNA sequences of two or more antigens, subunits or fragments thereof can be inserted into two different MVA insertion sites, one located on sMVA F1 and the other located on sMVA F2.

These insertion sites may include commonly used insertion sites such as the MVA deletion 2 (Del2) site, the intergenic region (IGR) between open reading frame (ORF) 44L and 45L (IGR44/45), the IGR between ORF 69R and 70L (IGR69/70), the IGR between 64L and 65L (IGR64/65), the Thymidine Kinase (TK) gene insertion site, or the MVA Deletion 3 (Del3) site, or any other MVA deletion site, intergenic region, or gene insertion site (ORF numbers are based on MVA strain Antoine (Accession #U94848)).

The sMVA or rsMVA expressing coronavirus antigens disclosed herein may be part of a vaccine composition that may be used in methods to treat or prevent viral infection. The vaccine composition as described herein may comprise a therapeutically effective amount of the sMVA or rsMVA as disclosed herein, and further comprising a pharmaceutically acceptable carrier according to a standard method. Examples of acceptable carriers include physiologically acceptable solutions, such as sterile saline and sterile buffered saline.

In some embodiments, the vaccine or pharmaceutical composition may be used in combination with a pharmaceutically effective amount of an adjuvant to enhance the prophylactic or therapeutic effects. Any immunologic adjuvant that may stimulate the immune system and increase the response to a vaccine, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynomycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosin α1. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the vaccine of the invention, as discussed above, may enhance the therapeutic effect achieved by the administration of the vaccine or pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

The vaccine or pharmaceutical composition containing the sMVA or rsMVA disclosed herein may be stored as an aqueous solution or a lyophilized product in a unit or multiple dose container such as a sealed ampoule or a vial.

The sMVA, rsMVA, vaccine or pharmaceutical composition disclosed herein can be used to stimulate SARS-CoV-2-specific humoral (binding antibodies, neutralizing antibodies) and cellular (CD4+ and CD8+ T cells) immune responses for the treatment or prevention of SARS-CoV-2 infection in animal models and humans. They also can be used to produce and isolate antibody responses that can be utilized for the treatment of or passive immunization against SARS-CoV-2 infections.

Disclosed herein is an sMVA vaccine composition. COH04S1 co-expresses SARS-CoV-2 Spike and Nucleocapsid, 2 antigens implicated in protective immunity, and shows very promising immune responses in mice, hamsters, ferrets and monkeys. Favorable pre-IND FDA response was received. FDA agreed that synthetic sMVA is equivalent to traditional MVA, and that No requirement for IND-directed tox studies. City of Hope's cGMP manufacturing facilities produced materials for Phase 1 and 2 studies. Up to 122 volunteers, age 18-54, were dosed, and 55 volunteers received either 1 or 2 doses.

The vaccine composition also comprises the N antigen, which elicits strong T cell response. The vaccine composition lacks gender dependency and is effective across age groups, e.g., from 2-year old to 75-year old. Strong immunogenicity was achieved even at lowest evaluated clinical dose. The vaccine composition achieved protection from severe disease in hamsters. The vaccine composition exhibits Th1-biased antibody and T cell response.

In some embodiments, the vaccine composition disclosed herein is formulated for nasal delivery for mucosal protection. Alternatively, the vaccine composition disclosed herein is formulated for intraperitoneal (IP), intramuscular (IM) or intranasal (IN) administration to induce strong immune responses in a subject. In certain embodiments, the vaccine composition is administered at 1×10⁷ PFU to 10×10⁸ PFU per dose, 1-10×10⁸ PFU per dose, or 1-5×10⁸ PFU per dose. In some embodiments, the vaccine composition is administered at about 1×10⁷ PFU per dose, about 2×10⁷ PFU per dose, about 3×10⁷ PFU per dose, about 4×10⁷ PFU per dose, about 5×10⁷ PFU per dose, about 6×10⁷ PFU per dose, about 7×10⁷ PFU per dose, about 8×10⁷ PFU per dose, about 9×10⁷ PFU per dose, about 1×10⁸ PFU per dose, about 2×10⁸ PFU per dose, about 3×10⁸ PFU per dose, about 4×10⁸ PFU per dose, about 5×10⁸ PFU per dose, about 6×10⁸ PFU per dose, about 7×10⁸ PFU per dose, about 8×10⁸ PFU per dose, about 9×10⁸ PFU per dose, or about 10×10⁸ PFU per dose. In certain embodiments, the vaccine composition is administered to a subject in a single dose. In certain embodiments, the vaccine composition is administered to a subject in a first dose, followed by a booster dose. The vaccine composition disclosed herein stimulates potent humoral and cellular immune responses against SARS-CoV-2 upon administration to a subject.

As demonstrated in the working examples, healthy adults immunized with COH04S1 at a dose of 1×10⁷ PFU developed binding antibodies to S, RBD, N and neutralizing antibodies, as well as functional T Cell responses to S and N antigens.

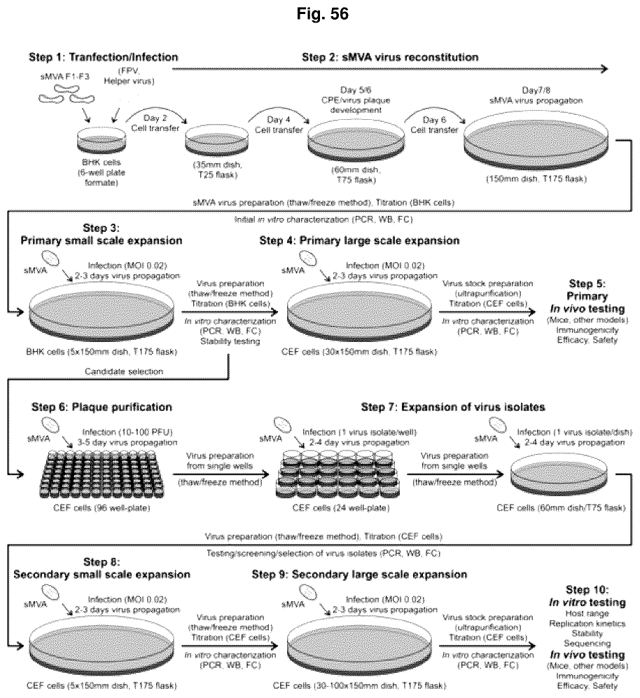

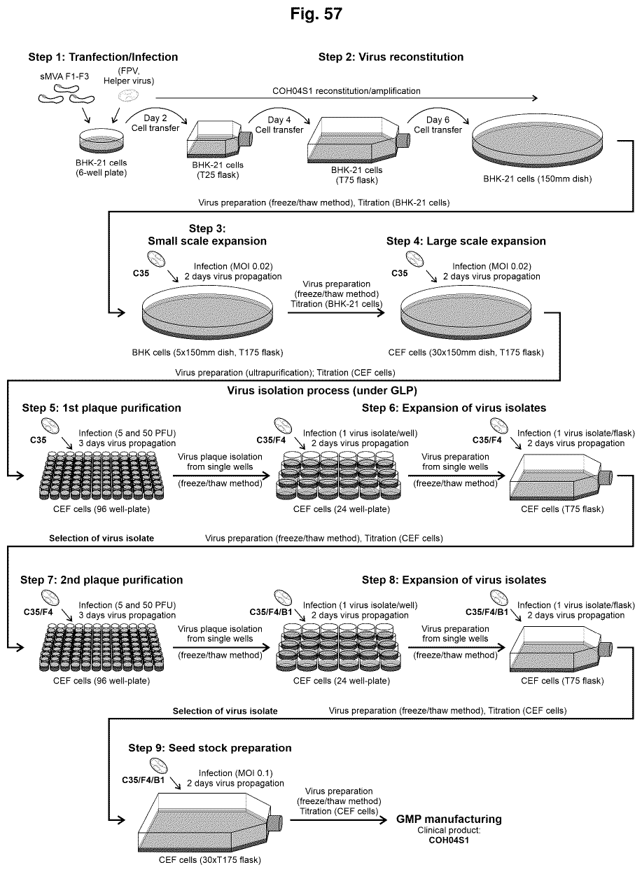

Also illustrated herein is pre-clinical vaccine production process from the initial virus reconstitution to the generation of the final pre-clinical virus stock, as illustrated in FIG. 56. The process includes the steps of transfection/infection with plasmids containing sMVA fragments, sMVA virus reconstitution, primary small scale expansion, primary large scale expansion, primary in vivo testing for immunogenicity, efficacy, and safety, plaque purification, expansion of virus isolates, secondary small scale expansion, secondary large scale expansion, and final in vitro and in vivo testing, as demonstrated in the working examples. This process can be further modified, improved, or optimized based on the production needs using common knowledge in the field.

Steps 1 and 2: Transfection/Infection and sMVA Virus Reconstitution

The three plasmids containing the three sMVA fragments F1-F3 (unmodified, modified, or a combination thereof) are isolated from E. coli by alkaline lysis. The isolated plasmids are co-transfected by Fugene HD lipid-based transfection reagent (Roche) into 60-70% confluent BHK-21 cells (ATCC® CCL-10™) that have been seeded the day before in a 6-well plate tissue culture format and grown in minimum essential medium (MEM, Gibco) with 10% fetal bovine serum (MEM10) at 37° C. in a 5% CO₂ incubator. At 4 hours post transfection, the BHK-21 cells are infected at approximately 0.1 to 1 multiplicity of infection (MOI) with FPV (ATCC VR-2553) to initiate sMVA virus transcription and reconstitution. The transfected/infected BHK-21 cells are incubated for 2 days in MEM10 in a 6-well tissue culture plate at 37° C. in a 5% CO₂ incubator and every other day transferred, re-seeded, and grown for two days in larger tissue culture plates over a period of 8-12 days as illustrated in FIG. 56 (Step 2) until most or all of the BHK-21 cells show signs of sMVA virus infection. Characteristic MVA viral plaque formation and cytopathic effects (CPEs) indicating sMVA virus reconstitution is usually detected at 4-8 days post transfection/infection. Fully infected BHK-21 cell monolayers are usually visible at 8-12 days post transfection/infection. sMVA virus from the infected BHK-21 cell monolayers are prepared in MEM with 2% FBS (MEM2) by 3 cycles of conventional freeze/thaw method and stored at −80° C. sMVA from these initial virus stocks is titrated on BHK-21 cells and usually characterized by various methods (PCR, Western Blot (WB), flow cytometry (FC)) to confirm sMVA reconstitution and antigen expression.

### Steps 3 and 4: Primary Small and Large Scale Expansion

To produce larger virus amounts for more vigorous in vitro and in vivo testing, the reconstituted sMVA virus from the initial virus stocks (Steps 1 and 2) is expanded in a two-step process, involving a first small-scale expansion on BHK-21 cells and a subsequent large-scale expansion on chicken embryo fibroblast (CEF) cells. For small scale expansion (step 3), BHK-21 cells seeded in 5×150 mm tissue culture dishes are allowed to grow to 80-90% confluency and infected at 0.02 MOI with the sMVA from the initial virus stocks. The infected BHK-21 cells are incubated for 2-3 days in MEM10 at 37° C. in a 5% CO₂ incubator. Virus stocks from the small-scale expansion are prepared by 3 cycles of freeze/thaw method, stored in MEM2 in a −80° C. freezer, and subsequently titrated on BHK-21 cells. sMVA virus from the small-scale expansion is characterized in vitro (PCR, WB, FC) to verify identity, genome reconstitution, and antigen expression. At this point of the development process, the sMVA virus may also undergo stability testing following propagation of 5-10 passages on CEF. For large scale expansion (step 4), freshly prepared CEF seeded in 30×150 mm tissue culture dishes are allowed to grow to 70-90% confluency and infected at 0.02 MOI with the sMVA virus prepared from the small-scale expansion. The infected CEF cells are grown for 2-3 days in MEM10 at 37° C. in a 5% CO₂ incubator. Virus from the large-scale expansion is prepared by 36% sucrose density ultracentrifugation, stored at −80° C. in 1 mM Tris-HCl (pH 9), and subsequently titrated on CEF cells. The purified virus is characterized in vitro by PCR, WB, and FC (or other methods) to confirm identity, fidelity of genome reconstitution, and antigen expression.

### Step 5: Primary In Vivo Testing

Following in vitro characterization, the purified virus from the large-scale expansion is used for in vivo studies to assess immunogenicity, protection against challenge, and safety of the vaccine candidates in different animal models. This may include studies in mice, but also studies in other animal models such as hamsters, ferrets, or non-human primates. Dose escalation and immunization routes can be tested to assess optimal conditions for immunogenicity, and protection against viral challenge.

### Steps 6 and 7: Plaque Purification and Expansion of Virus Isolates

For the transition into clinical production, selected sMVA vaccine constructs (selected based on results under step 4 and 5) are plaque purified, expanded, and re-tested by in vitro and in vivo studies. From this point on, all steps of the production process are conducted under serum-free conditions using VP-SFM medium (Gibco). For the plaque purification procedure (Step 6), freshly prepared CEF cells (80-90% confluent) seeded the day before in a 96-well tissue culture plate are infected at 10-100 PFU/plate with sMVA virus from the primary small-scale expansion (step 3). At 3-5 days post infection, the CEF cells of the 96-well plates are screened for single viral plaque formation per well and sMVA virus isolates from single wells are prepared by 3 cycles of freeze/thaw method. The virus isolates prepared from single wells are then expanded though infection of 80-90% confluent CEF cells seeded in 24-well tissue culture plate (1 virus isolate/well; Step 7, FIG. 56). At 2-4 days post infection, the virus isolates expanded in the 24-well plates are prepared by freeze/thaw method and further expanded at 1 isolate/dish on 80-90% confluent CEF seeded in 60 mm tissue culture dishes. The infected CEF cells are grown for 2-4 days and the expanded virus isolates harvested by freeze/thaw method and titrated on CEF. The titrated virus isolates (5-10) are then screened by in vitro testing using PCR, WB and FC to evaluate the identity, genome composition, and antigen expression of the single virus isolates.

### Steps 8 and 9: Secondary Small and Large Scale Expansion

As a next step, selected plaque purified virus isolates of the sMVA vaccine candidates are further expanded in a two-step process involving a secondary small scale and secondary large scale expansion to produce large amounts of virus for vigorous in vitro and in vivo testing of the final isolates. The secondary expansion procedure principally follows the primary expansion procedure of the pre-clinical vaccine development process (FIG. 56, Steps 3 and 4 and steps 8 and 9), except that the secondary expansion procedure uses CEF cells grown exclusively under serum-free conditions (VP-SFM). Virus stocks from the large scale expansion are prepared by ultracentrifugation and stored at −80° C. in 1 mM Tris-HCl (pH 9). Final purified virus stocks are characterized in vitro by PCR, WB and FC to confirm the identity, genome composition, and antigen expression of the selected virus isolates of the sMVA vaccine candidates.

### Step 10: Final In Vitro and In Vivo Testing

Following initial in vitro testing of the final products, the selected virus isolates are further evaluated in vitro for host range, replication kinetics, vaccine stability, and sequencing of the complete genome. In addition, immunogenicity, protection against challenge, and safety of the final virus isolates of the sMVA vaccine candidates are investigated in animal models (mice, or other animals).

Also disclosed herein are various prime-boost procedures. In some embodiments, a prime-boost procedure comprises a first and second immunizations or additional booster immunizations by the same sMVA vector encoding two or more SARS-CoV-2 antigen sequences of the Wuhan-Hu-1 reference strain or different variants of concern. In some embodiments, a prime-boost procedure comprises a first and second immunization or additional booster immunizations by a mixture of two or more sMVA vectors that encode two or more different SARS-CoV-2 antigen sequences selected from the Wuhan-Hu-1 reference strain and different variants of concern. In some embodiments, a prime-boost procedure that includes a first immunization with a sMVA vector encoding one or more SARS-CoV-2 antigen sequences of the Wuhan-Hu-1 reference strain and a second immunization with different sMVA vector encoding one or more SARS-CoV-2 antigen sequences of different variants of concern, or vice versa. In some embodiments, a prime-boost procedure comprises multiple immunization with an sMVA vector encoding one or more SARS-CoV-2 antigen sequences of the Wuhan-Hu-1 reference strain and multiple booster immunization with different sMVA vector encoding one or more SARS-CoV-2 antigen sequences of different variants of concern, or vice versa.

In this disclosure, COH04S1 has an sMVA-N/S vector construction as illustrated in FIG. 5 and FIG. 17. COH04S1 is the clinical product derived by double-plaque purification from the parental sMVA-N/S vector C35 (a.k.a., sMVA-N/S tv) as illustrated in FIG. 57. As used herein, the terms “sMVA-CoV2 vector” and “sMVA-SARS-CoV2 vector” may be used interchangeably to refer to sMVA vectors expressing one or more SARS-CoV2 antigens.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Materials and Methods

Cells and Viruses: BHK-21 (CCL-10), A549 (CCL-185), HeLa (CCL-2), 293T (CRL-1573), 143B (CRL-8303), MRC-5 (CCL-171), HEK293/17 (CRL11268), THP-1 (TIB-202), ARPE-19 (CRL-2302) were purchased from the American Type Culture Collection (ATCC) and grown according to ATCC recommendations. CEF were purchased from Charles River (10100795) and grown in minimum essential medium (MEM) with 10% FBS (MEM10). HEK293T/ACE2 were a kind gift of Pamela J. Bjorkman⁴⁶. The wtMVA (NIH Clone 1) was used solely as a reference standard. To produce sMVA and wtMVA virus stocks, CEF were seeded in 30×150 mm tissue culture dishes, grown to about 70-90% confluency, infected at 0.02 multiplicity of infection (MOI) with sMVA or wtMVA. Two days post infection, purified virus was prepared by 36% sucrose density ultracentrifugation and virus resuspension in 1 mM Tris-HCl (pH 9)⁴⁷. Virus stocks were stored at −80° C. Virus titers were determined on CEF by immunostaining of viral plaques at 16-24 hours post infection using polyclonal Vaccinia antibody. FPV stocks were produced following propagation on CEF using FPV strain TROVAC (ATCC VR-2553)³ or HP1.441⁴, kindly provided by Bernard Moss. FPV titers were evaluated on CEF by virus plaque determination. SARS-CoV-2 strain USA-WA1/2020 (BEI Resources NR-52281) was used in the focus reduction neutralization test (FRNT) assay⁵³.

Construction of sMVA fragments: The three about 60 kbp sMVA fragments (F1-F3; FIG. 1) comprising the complete MVA genome sequence reported by Antoine et al. (NCBI Accession #U94848) 4 were constructed as follows: sMVA F1 contained base pairs 191-59743 of the MVA genome sequence; sMVA F2 comprised base pairs 56744-119298 of the MVA sequence; and sMVA F3 included base pairs 116299-177898 of the reported MVA genome sequence⁴. A CR/HL/CR sequence arrangement composed of

(SEQ ID NO: 66)
5′-TTT TTT TCT AGA CAC TAA ATA AAT AGT AAG
ATT AAA TTA ATT ATA AAA TTA TGT ATA TAA TAT
TAA TTA TAA AAT TAT GTA TAT GAT TTA CTA ACT
TTA GTT AGA TAA ATT AAT AAT ACA TAA ATT TTA
GTA TAT TAA TAT TAT AAA TTA ATA ATA CAT AAA
TTT TAG TAT ATT AAT ATT ATA TTT TAA ATA TTT
ATT TAG TGT CTA GAA AAA AA-3′

was added in the same orientation to both ends of each of the sMVA fragments, wherein the italicized letters indicate the duplex copy of the MVA terminal HL sequence and the underlined letters indicate the CR sequences. Notably, the CR/HL/CR sequences incorporated at the ITRs of sMVA F1 and F3 were added in identical arrangement as the CR/HL/CR sequences occur at the ITRs at the genomic junctions of putative MVA replication intermediates⁴. The sMVA fragments were produced and assembled by Genscript using chemical synthesis, combined with a yeast recombination system. All sMVA fragments were cloned into a yeast shuttle vector, termed pCCI-Brick, which contains a mini-F replicon for stable propagation of large DNA fragments as low copy BACs in E. coli. sMVA F1 and F3 were cloned and maintained in EPI300 E. coli (Epicentre), while sMVA F1 was cloned and maintained in DH10B E. coli (Invitrogen).

Antigen insertion: SARS-CoV-2 S and N antigen sequences were inserted into the sMVA fragments by En passant mutagenesis in GS1783 E. coli cells^{48,49}. Briefly, transfer constructs were generated that consisted of the S or N antigen sequence with upstream mH5 promoter sequence and downstream Vaccinia transcription termination signal (TTTTTAT, SEQ ID NO: 67), and a kanamycin resistance cassette flanked by a 50 bp gene duplication was introduced into the antigen sequences. The transfer constructs were amplified by PCR with primers providing about 50 bp extensions for homologous recombination and the resulting PCR products were used to insert the transfer constructs into the sMVA DNA by a first Red-recombination reaction^{48,49}. Primers

(SEQ ID NO: 68)
5′-AAA AAA TAT ATT ATT TTT ATG TTA TTT TGT
TAA AAA TAA TCA TCG AAT ACG AAC TAG TAT AAA
AAG GCG CGC C-3′
and
(SEQ ID NO: 69)
5′-GAA GAT ACC AAA ATA GTA AAG ATT TTG CTA
TTC AGT GGA CTG GAT GAT TCA AAA ATT GAA AAT
AAA TAC AAA GGT TC-3′

were used to insert the N antigen sequence into the Del2 site. Primers

(SEQ ID NO: 70)
5′-ATA TGA ATA TGA TTT CAG ATA CTA TAT TTG
TTC CTG TAG ATA ATA ACT AAA AAT TTT TAT CTA
GTA TAA AAA GGC GCG CC-3′
and
(SEQ ID NO: 71)
5′-GGA AAA TTT TTC ATC TCT AAA AAA AGA TGT
GGT CAT TAG AGT TTG ATT TTT ATA AAA ATT GAA
AAT AAA TAC AAA GGT TC-3′

were used to insert the S antigen sequence into the IGR69/90 insertion site primers. Primers

(SEQ ID NO: 72)
5′-TTG GGG AAA TAT GAA CCT GAC ATG ATT AAG
ATT GCT CTT TCG GTG GCT GGT AAA AAA TTG AAA
ATA AAT ACA AAG GTT C-3′
and
(SEQ ID NO: 73)
5′-ACA AAA TTA TGT ATT TTG TTC TAT CAA CTA
CCT ATA AAA CTT TCC AAA TAC TAG TAT AAA AAG
GCG CGC C-3′

were used to insert the S or N antigen sequence into the Del3 site. Underlined letters indicate the sequences used to produce about 50 bp extensions for homologous recombination. The S and N antigen sequences were based on the SARS-CoV-2 reference strain (NCBI Accession #NC_045512) and codon-optimized for Vaccinia^{10,38}. Codon-optimized S and N gene sequences were synthesized by Twist Biosciences. The transfer constructs were amplified by PCR with Phusion polymerase (Thermo Fisher Scientific) using primers providing ˜50 bp extensions for homologous recombination to insert the transfer constructs into the sMVA fragments by Red-recombination. Inserted antigen sequences were verified by PCR, restriction enzyme digestion, and sequencing. The amplified PCR products were purified using the NucleoSpin Gel and PCR clean-up kit (Macherey-Nagel) and 100 ng of PCR product was electroporated at 15 kV/cm, 25 μF, and 200Ω into 50 μL of recombination-competent GS1783 bacteria harboring the sMVA fragments. The bacteria were re-suspended in 1 mL of Luria-Bertani (LB) medium without antibiotics and incubated for 2 h at 32° C. and 220 r.p.m. After 2 h of incubation, the bacteria were streaked onto LB agar plates with 30 μg/mL chloramphenicol and 30 μg/mL kanamycin and incubated at 32° C. for 2 days. Bacterial clones harboring sMVA fragments with inserted antigen sequences at the respective MVA insertion sites were identified by PCR and restriction pattern analysis. To seamlessly remove the kanamycin resistance marker from the inserted antigen sequences by a I-SceI-mediated second Red-recombination reaction, 100 μL of an overnight culture of selected bacterial clones was added to 900 μL of LB medium containing 30 μg/mL chloramphenicol and incubated for 1.5-2 h at 32° C. and 220 r.p.m. Subsequently, 1 mL of LB containing 30 μg/mL chloramphenicol and 2% L-arabinose was added to induce the expression of the I-SceI homing endonuclease enzyme and to induce a double-strand break at the 50 bp gene duplication. The bacteria were incubated for 1 h at 32° C. and then transferred to a water bath and incubated for 30 min at 220 r.p.m. and 42° C. to induce the expression of the Red-recombination proteins and to mediate the removal of the kanamycin resistance marker by recombination of the 50 bp gene duplication. After an additional incubation period of the bacteria for 2 h at 32° C. and 220 r.p.m., the bacteria were streaked onto LB agar plates with 30 μg/mL chloramphenicol and 1% L-arabinose and incubated at 32° C. for 2 days. Bacterial clones harboring sMVA fragments with seamlessly removed kanamycin marker from the inserted antigen sequences were identified by PCR, restriction pattern analysis, and Sanger sequencing.

sMVA virus reconstitution: sMVA virus reconstitution from the three sMVA DNA plasmids in BHK-21 cells using FPV as a helper virus was performed as follows⁸⁻¹⁰. The three sMVA DNA plasmids were isolated from E. coli by alkaline lysis⁵⁰ and co-transfected into 60-70% confluent BHK-21 cells grown in 6-well plate tissue culture plates using Fugene HD transfection reagent (Roche) according to the manufacturer's instructions. At 4 hours post transfection, the cells were infected with approximately 0.1-1 MOI of FPV to initiate the sMVA virus reconstitution. The transfected/infected BHK-21 cells were grown for 2 days and then every other day transferred, re-seeded, and grown for additional two days in larger tissue culture formats over a period of 8-12 days until most or all of the cells showed signs of sMVA virus infection. Using this procedure, characteristic MVA viral plaque formation and cytopathic effects (CPEs) indicating sMVA virus reconstitution was usually detected at 4-8 days post transfection/infection. Fully infected BHK-21 cell monolayers were usually visible at 8-12 days post transfection/infection. sMVA virus from infected BHK-21 cell monolayers was prepared by conventional freeze/thaw method and passaged once on BHK-21 cells before producing purified virus stocks on CEF. sMVA or recombinant sMVA-CoV-2 vectors were reconstituted either with FPV HP1.441 (sMVA hp, sMVA-N/S, sMVA-S/N hp) or TROVAC (sMVA tv1 and tv2, sMVA-S tv, sMVA-N tv, sMVA-N/S tv, sMVA-S/N tv).

Host cell range: sMVA and wtMVA host cell range using various human cell lines (HeLa, 293T, MRC-5, A549, and 143B) BHK-21 cells, and CEF was determined as follows. The cells were seeded in 6-well plate tissue culture format and at 70-90% confluency infected in duplicates with 0.01 MOI of sMVA or wtMVA using MEM2. At 2 hours post infection, the cells were washed twice with PBS and incubated for two days in normal growth medium (as described under cells and viruses). After the incubation period, virus was prepared by conventional freeze/thaw method and the virus titers of each duplicate infection were determined in duplicate on CEF.

Replication kinetics: To compare the replication kinetics of sMVA and wtMVA, CEF or BHK-21 cells were seeded in 6 well-plate tissue culture format and at 70-90% confluency infected in triplicates at 0.02 MOI with sMVA or wtMVA using MEM2. After 2 hours of incubation, the cells were grown in MEM10. At 24 and 48 hours post infection, virus was prepared by freeze/thaw method and the virus titers of each triplicate infection and the inoculum was determined in duplicate on CEF.

Plaque size analysis: To compare the plaque size of sMVA virus and wtMVA, CEF or BHK-21 cells were seeded in 6-well plate tissue culture format and at 70-90% confluency infected with 0.002 MOI with sMVA or wtMVA using MEM2. After 2 hours of incubation, MEM10 was added and the cells were grown for 16-24 hours. The cell monolayers were stained with Vaccinia virus polyclonal antibody and viral plaques were imaged using Leica DMi8 inverted microscope and measured using LAS X software. The size of 25 viral plaques per sMVA or wtMVA was calculated using the formula Area=π×a×b, where a and b are the major and minor radius of the ellipse, respectively.

PCR analysis: To characterize the viral DNA of the sMVA vectors by PCR, CEF were seeded in 6-well plate tissue culture format and at 70-90% confluency infected at 5 MOI with sMVA or wtMVA. DNA was extracted at 16-24 hours post infection by the DNA Easy Blood and Tissue Kit (Qiagen) according to the manufacturer's instructions. All PCR reactions were performed with Phusion polymerase (ThermoFisher Scientific). Primers 5′-TCG TGG TGT GCC TGA ATC G-3′ and 5′-AGG TAG CGA CTT CAG GTT TCT T-3′ (SEQ ID NO: 74) were used to detect MVA ITR sequences; primers 5′-TAT CCA CCA ATC CGA GAC CA-3′ and 5′-CCT CTG GAC CGC ATA ATC TG-3′ (SEQ ID NO: 75) were used to verify the transition from the left ITR into the unique region; primers 5′-AGG TTT GAT CGT TGT CAT TTC TCC-3′ and 5′-AGA GGG ATA TTA AGT CGA TAG CCG-3′ (SEQ ID NO: 76) were used to verify the Del2 site with or without inserted N antigen sequence; primers 5′-TGG AAT GCG TTC CTT GTG C-3′ (SEQ ID NO: 77) and 5′-CGT TTT TCC CAT TCG ATA CAG-3′ (SEQ ID NO: 78) with binding sites flanking the F1/F2 homologous sequences were used to verify the F1/F2 recombination site; primers 5′-TAT AGT CTT TGT GGC ATC CGT TG-3′ (SEQ ID NO: 79) and 5′-ACC CAA ACT TTA GTA AGG CCA TG-3′ (SEQ ID NO: 80) were used to verify the IGR69/70 insertion site with or without inserted S antigen; primers 5′-ATA AGC GTT GTC AAA GCG GG-3′ (SEQ ID NO: 81) and 5′-AGG AAA TAG AAA TTG TTG GTG CG-3′ (SEQ ID NO: 82) with binding sites flanking the F2/F3 homologous sequences were used to verify the F2/F3 recombination site; primers 5′-ACA TTG GOG GAC AAT CTA AAA AC-3′ (SEQ ID NO: 83) and 5′-ATC ATC GGT GGT TGA TTT AGT AGT G-3′ (SEQ ID NO: 84) were used to verify the Del3 insertion site with and without inserted S or N antigen sequences; primers 5′-TAT CCA CCA ATC CGA GAC CA-3′ (SEQ ID NO: 85) and 5′-GTC TGT CCG TCT TCT CTA TTG TTT A-3′ (SEQ ID NO: 86) were used to verify the transition from the unique region into the right ITR; and primers 5′-TTA ACT CAG TTT CAA TAC GGT GCA G-3 (SEQ ID NO: 87) and 5′-TGG GGT TTC TTC TCA GGC TAT C-3′ (SEQ ID NO: 88) were used to detect the SopA element of the BAC vector. PCR products were analyzed by agarose gel electrophoresis and imaged using Syngene PXi6 imager with GeneSys (v1.5.4.0) software. Uncropped gel images are provided as Source Data file. To sequence the PCR products derived from the sMVA vectors, the amplified PCR products were purified using the NucleoSpin Gel and PCR Clean-up Kit (Macherey-Nagel) according to the manufacturer's instructions and analyzed by Sanger sequencing.

Restriction pattern analysis: BHK-21 cells were seeded in 20×150 mm tissue culture dishes, grown to about 70-90% confluency, and infected at 0.01 MOI with wtMVA, sMVA tv1, or sMVA tv2. The purified virus was prepared two days post-infection as previously described⁴⁷. Viral DNA (vDNA) was phenol/chloroform extracted, followed by ethanol precipitation as previously described⁵¹. Briefly, isolated virus particles were resuspended in lysis buffer (50 mM Tris-HCl PH 8.0, 1.2% SDS, 4 mM EDTA pH 8.0, 4 mM CaCl2, and 0.4 mg/mL proteinase K) and incubated overnight at 37° C. DNA was extracted twice with phenol; each extraction was performed by adding an equal volume of buffered phenol and centrifugation at room temperature (RT) for 10 min at 300×g. Aqueous phase was carefully collected to avoid DNA shearing. Final extraction was performed by adding equal volume of 1:1 phenol/chloroform to aqueous phase, followed by centrifugation as described above, and completed by ethanol precipitation of phenol/chloroform extracted viral DNA. DNA concentration and A260/A280 ratios were determined using Nano Vue (GE Healthcare Bio-sciences Corp). 10 μg of vDNA were digested with 3 units of either KpnI or XhoI, followed by visualization on 0.5% EtBr-stained agarose gel that was run at 2.4v/cm, overnight. Images were acquired using Syngene PXi6 imager with GeneSys (v1.5.4.0) software.

Sequencing of sMVA fragments and sMVA vectors: PacBio (Pacific Biosciences) Long Read Sequencing analysis was used to determine the sequences of the cloned sMVA fragments (F1-F3) and reconstituted sMVA vectors. Plasmid DNA for sequencing the sMVA fragments was isolated by QIAGEN Large-Construct Kit according to the manufacturer's instructions. Viral DNA for sequencing sMVA was isolated from purified virus particles by phenol/chloroform extraction as disclosed above. Viral DNA for sequencing the sMVA-CoV2 vectors was isolated from purified virus particles by NucleoSpin Blood QuickPure DNA extraction kit (Macherey-Nagel) according the manufacturer's instructions. Briefly, 5 μg of fragmented DNA was converted to barcoded SMRTbell libraries using the SMRTbell Template Prep Kit 1.0 and Barcoded Adapter Plate-96 (PacBio). Libraries of the sMVA fragments and sMVA vector were size-selected (7-kb size cutoff) with BluePippin (Sage Science). After polymerase binding to the libraries with sequencing primers, the polymerase complexes were loaded into RSII SMRT cells using MagBeads loading and sequenced on PacBio RSII with 6 h movie. The polymerase complexes of sMVA-CoV2 vectors were loaded into a Sequel SMRT cell using diffusion mode and sequenced on PacBio Sequel with 10 h movie. Read demultiplexing, read mapping to the reference sequences, and Circular Consensus Sequencing (CCS) analyses were performed by Demultiplex Barcodes, Resequencing, and CCS modules, respectively, either in SMRT Portal (v. 2.3.0) or SMRT Link (v6.0.0.47841) or SMRT Link (v8.0.0.80529). The variants calling with CCS reads were carried out using VarScan v2.3.9 after mapping CCS reads using pbmm2v 1.0.0. De novo assembly was done using canu v1.7.1. The 5′ start position of the assembled contig was edited by comparing to the references. MVA U94848.1 was used as a reference for mapping the reads of the sMVA genome sequence. Sequences of the sMVA fragments and sMVA-CoV2 vectors were mapped via alignment with corresponding reference sequences based on MVA U94848.1 that were constructed by Vector NTI (Invitrogen, v. 11.5). Along with the comparison of de novo assembled contig to each reference, this analysis confirmed the sequence identity of the cloned sMVA fragments and reconstituted sMVA vectors, including a single point mutation in a non-coding determining region at 3 base pairs downstream of 021L4 that was found in sMVA fragment F1 and all sequenced reconstituted sMVA vectors (sMVA and sMVA-CoV-2 vectors). An additional variation (point mutation) that could not be unambiguously excluded was found in a non-coding determining region at the tandem repeats 88 bp from the end of the ITR within sMVA fragment F3. As these two variations were present in the cloned sMVA fragments, they were confirmed as errors originating during the chemical synthesis of the sMVA fragments. The internal unique region and unique regions of the ITRs encompassing the complete MVA coding content could be reliably assembled for all reconstituted sMVA vectors. The sequence contig of the sMVA vector covered almost (over 99%) the complete U94848.1 reference sequence, with only a few exceptions at the highly repetitive ITR tandem repeats. The complete regions of the ITR tandem repeats of the sMVA-CoV2 vectors could not be reliably mapped through alignment with the reference sequences or de novo assembly due to low coverage at these regions, likely as a result of the quality of the sequence reads. Reference sequences of the sMVA fragments and sMVA-CoV2 vectors based on the PacBio sequencing were deposited in NCBI. To determine the absence of contaminating BAC vector sequences in the raw sequencing data of the reconstituted sMVA vectors, the sequencing reads were aligned onto the reference pCCI-Brick vector sequence provided by Genscript using the resequencing module in SMRT Link (v8.0.0.80529).

Immunoblot analysis: BHK-21 cells infected at 5 MOI were harvested 24-hours post infection. Proteins were solubilized in PBS with 0.1% Triton X-100, supplemented with protease inhibitor, then reduced and denatured in Laemmli buffer containing DTT and boiled at 95° C. for about 10 minutes. Proteins were resolved on a 4-20% Mini Protean TGX gradient gel (BioRad), and transferred onto PVDF membrane. S protein was probed with anti-SARS-CoV-1 S1 subunit rabbit polyclonal antibody (40150-T62-CoV2, Sino Biological); N protein was probed with anti-SARS-CoV1 NP rabbit polyclonal antibody (40413-T62, Sino Biological). Vaccinia BR5 protein was probed as a loading control. Anti-rabbit polyclonal antibody conjugated with horseradish peroxidase (Sigma-Aldrich) was used as a secondary antibody and protein bands were visualized with chemiluminescent substrate (ThermoFisher).

Flow cytometry: Hela cells were seeded in a 6-well plate (5×10⁵/well) and infected the following day with sMVA vaccine candidates at an MOI of 5. Following an incubation of 6 hours, cells were detached with non-enzymatic cell dissociation buffer (Cat. No. 13151014, GIBCO). Cells were either incubated directly with primary antibody or fixed and permeabilized prior to antibody addition. Anti-SARS-CoV-1 S1 mouse (40150-R007, Sino Biological) and S2 rabbit (GTX632604, GeneTex) monoclonal antibodies, anti-SARS-CoV-1 N rabbit monoclonal antibody (40143-R001, Sino Biological), and anti-vaccinia rabbit polyclonal antibody (9503-2057, Bio Rad) were used in dilution 1:2,000. One hour later anti-mouse or anti-rabbit Alexa Fluor 488-conjugated secondary antibodies (A11001, A21206; Invitrogen) were added to the cells at a dilution of 1:4,000. Live cells were ultimately fixed with 1% paraformaldehyde (PFA) and acquired using a BD FACSCelesta flow cytometer with BD FACSDiva software (v8.0.1.1). Analysis was performed using FlowJo (v10.6.2).

Immunofluorescence: BHK-21 or Hela cells were grown on glass coverslips and infected with sMVA or recombinant sMVAs encoding S and/or N proteins at an MOI of 5 for 6 hours at 37° C. in a humidified incubator (5% CO₂). After infection, cells were fixed for 15 minutes in 2% PFA and then directly permeabilized by addition of ice cold 1:1 acetone/methanol for 5 minutes on ice. Cells were blocked for 1 hour with 3% BSA at room temperature, incubated with primary antibody mix (1:500) against the S2 subunit or N for 1 hour at 37° C., and then incubated with Alexa-conjugated secondary antibodies (ThermoFisher) (1:2000) for 1 hour at 37° C., with washing (PBS+0.1% Tween20) between each step. For detection of cell membranes and nuclei, cells were incubated with Alexa-conjugated wheat germ agglutinin at 5 μg/mL (ThermoFisher) and DAPI for 10 minutes at room temperature. Coverslips were washed and mounted onto slides with Fluoromount-G (SouthernBiotech). Microscopic analysis was performed using a laser-scanning confocal microscope (Zeiss, LSM700). Images were acquired and processed using Zen software (Zeiss, Black Edition Version 8.1).

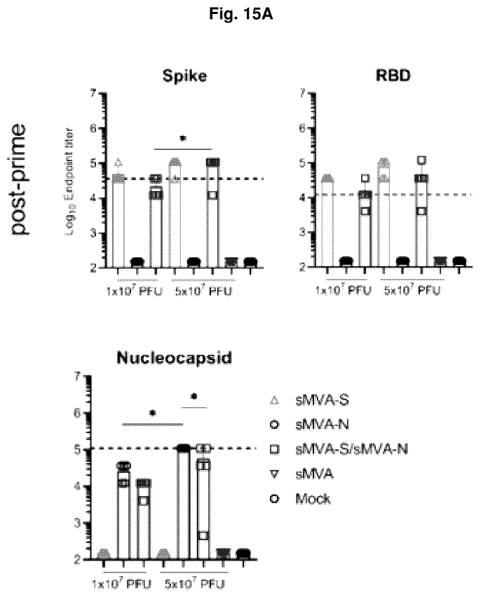

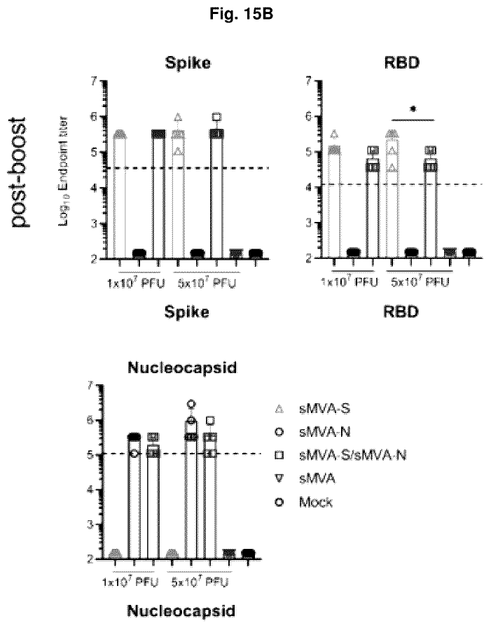

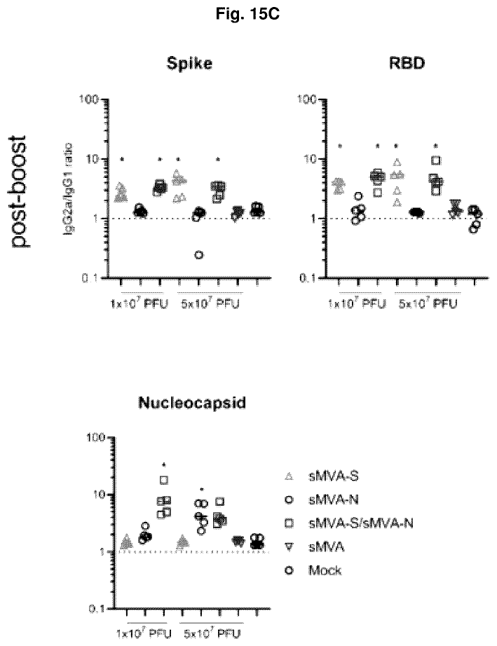

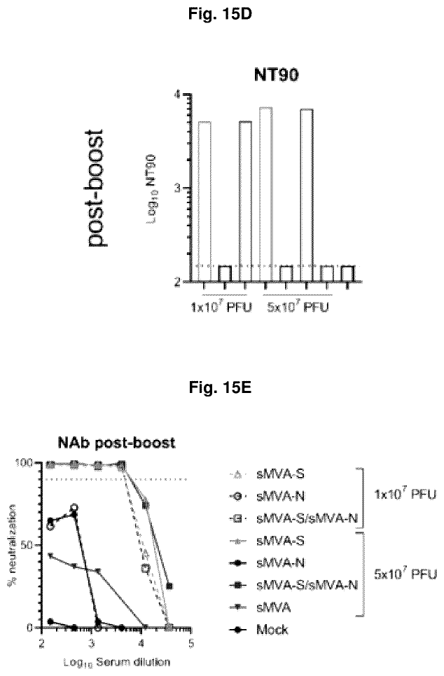

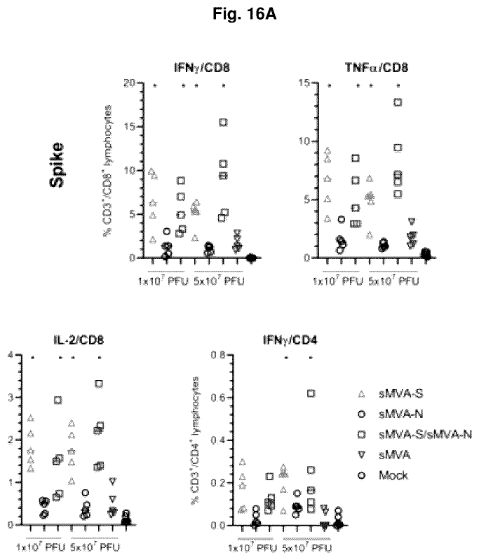

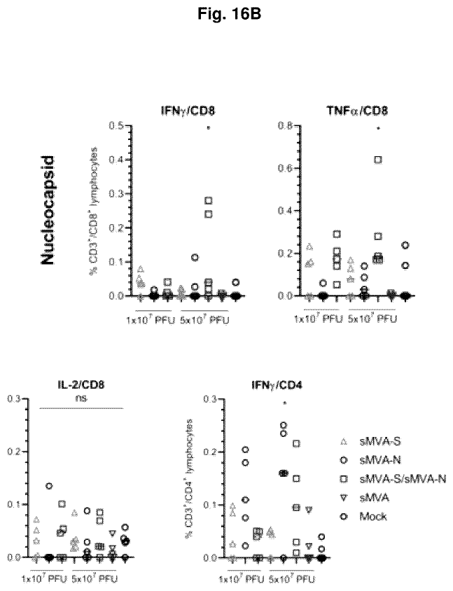

Mouse immunization: The Institutional Animal Care and Use Committee (IACUC) of the Beckman Research Institute of City of Hope (COH) approved protocol 20013 assigned for this study. All study procedures were carried out in strict accordance with the recommendations in the Guide for the Care and Use of Laboratory Animals and the Public Health Service Policy on the Humane Care and Use of Laboratory Animals. 6 weeks old C57BL/6 (C57BL/6J, 000664) or Balb/c (BALB/cJ, 000651) were purchased from the Jackson laboratories. C57BL/6 Nramp were bred at the City of Hope animal facility. Mice (N=4-5) were immunized twice in three-week intervals by intraperitoneal route with 5×10⁷ PFU (high dose) or 1×10⁷ PFU (low dose) of sMVA, wtMVA, or sMVA-CoV2 vectors. To determine immune stimulation by both the S and N antigen when using separate vectors (FIGS. 15-16), mice were co-immunized via the same immunization schedule and route with half of the high (2.5×10⁷ PFU) or low dose (0.5×10⁷ PFU) of each of the vaccine vectors. Blood samples for humoral immune analysis were collected by retro-orbital bleeding two weeks post-prime and one-week post booster immunization. Splenocytes for cellular immune analysis were collected at one-week post booster immunization and were isolated by standard procedure after animals were humanely euthanized.

Binding antibodies: Binding antibodies in mice immunized with sMVA, wtMVA, or sMVA-CoV2 vectors were evaluated by ELISA. ELISA plates (3361, Corning) were coated overnight with 1 μg/ml of MVA expressing Venus fluorescent marker⁹, S (S1+S2, 40589-V08B1, Sino Biological), RBD (40592-V08H, Sino Biological) or N (40588-V08B, Sino Biological). Plates were blocked with 3% BSA in PBS for 2 hours. Serial dilutions of the mouse sera were prepared in PBS and added to the plates for two hours. After washing, 1:3,000 dilution of HRP-conjugated anti-mouse IgG secondary antibody (W402B, Promega) was added and incubated for one additional hour. Plates were developed using 1-Step Ultra TMB-ELISA (34028, Thermo Scientific) for one to two minutes after which the reaction was stopped with 1M H2SO4. Plates were read at 450 nm wave length using FilterMax F3 microplate reader (Molecular Devices). Binding antibody endpoint titers were calculated as the last serum dilution to have an absorbance higher than 0.1 absorbance units (OD) or higher than the average OD in mock immunized mice plus 5 times the standard deviation of the OD in the same group at the same dilution. For evaluation of the IgG2a/IgG1 ratio, mouse sera were diluted 1:10,000 in PBS. The assay was performed as described above except for the secondary antibodies (1:2,000. goat Anti-Mouse IgG2a cross absorbed HRP antibody, Southern biotech, 1083-05; Goat anti-Mouse IgG1 cross absorbed HRP antibody, Thermo Scientific, A10551). The IgG2a/IgG1 ratio was calculated by dividing the absorbance read in the well incubated with the IgG2a secondary antibody divided by the absorbance for the same sample incubated with the IgG1 antibody.

MVA neutralization assay: ARPE-19 cells were seeded in 96 well plates (1.5×10⁴ cells/well). The following day, serial dilutions of mouse sera were incubated for 2 hours with MVA expressing the fluorescent marker Venus10 (1.5×10⁴ PFU/well). The serum-virus mixture was added to the cells in duplicate wells and incubated for 24 hours. After the 24-hour incubation period, the cells were imaged using a Leica DMi8 inverted microscope. Pictures from each well were processed using Image-Pro Premier (Media Cybernetics) and the fluorescent area corresponding to the area covered by MVA-Venus infected cells was calculated.

SARS-CoV-2 pseudovirus production: The day before transfection, HEK293T/17 were seeded in a 15 cm dish at a density of 5×10⁶ cells in DMEM supplemented with 10% heat inactivated FBS, non-essential amino acids, HEPES, and glutamine⁵². Next day, cells were transfected with a mix of packaging vector (pALDI-Lenti System, Aldevron), luciferase reporter vector and a plasmid encoding for the wild type SARS-CoV2 Spike protein (Sino Biological) or vesicular stomatitis virus G (VSV-G, Aldevron), using FuGENE6 (Roche) as a transfection reagent:DNA ratio of 3:1, according to manufacturer's protocol. Sixteen hours post-transfection, the media was replaced and cells were incubated for an additional 24-72 hours. Media were harvested at 24-, 48- and 72 hours, clarified by centrifugation at 1,500 RPM for 5 minutes and filtered using a sterile 0.22 μm pore size filter. Clarified lentiviral particles were concentrated by ultracentrifugation at 20,000 RPM for 2 hours at 4° C. The pellet was resuspended in DMEM containing 2% heat inactivated-FBS and stored overnight at 4° C. to allow the pellet to completely dissolve. Next day, samples were aliquoted, snap frozen and stored at −80° C. for downstream assays.

SARS-CoV-2 pseudotype neutralization and ADE assay: Levels of p24 antigen in the purified SARS-CoV-2 pseudotype suspension were measured by ELISA (Takara). Mouse sera were heat inactivated, pooled and diluted at a linear range of 1:100 to 1:50,000 in complete DMEM. For the neutralization assay, diluted serum samples were pre-incubated overnight at 4° C. with SARS-CoV-2-Spike pseudotyped luciferase lentiviral vector, normalized to 100 ng/ml of p24 antigen. HEK293T cells overexpressing ACE-2 receptor were seeded the day before transduction at a density of 2×10⁵ cells per well in a 96-well plate in complete DMEM. Before infection, 5 μg/mL of polybrene was added to each well. Neutralized serum samples were then added to the wells and the cells were incubated for an additional 48 hours at 37° C. and 5% CO₂ atmosphere. Following incubation, cells were lysed using 40 μL of Luciferase Cell Culture Lysis 5× Reagent per well (Promega). Luciferase activity was quantified using 100 μL of Luciferase Assay Reagent (Promega) as a substrate. Relative luciferase units (RLU) were measured using a microplate reader (SpectraMax L, Molecular Devices) at a 570 nm wave length. The percent neutralization titer for each dilution was calculated as follows: NT=[1−(mean luminescence with immune sera/mean luminescence without immune sera)]×100. The titers that gave 90% neutralization (NT90) were calculated by determining the linear slope of the graph plotting NT versus serum dilution by using the next higher and lower NT. In all the experiments RLU in uninfected cells was measured and was always between 50 and 90.

For the ADE assay, THP1 cells were seeded at a confluency of 2×10⁶ cells/mL in a 96 well plate and co-incubated for 48 hours with serum samples diluted at 1:5,000 or 1:50,000 in the presence of SARS-CoV-2-Spike pseudotyped or VSV-G luciferase lentiviral vector, normalized to 100 ng/ml of p24 antigen. Following incubation, cells were lysed using 100 μL of ONE-Glo Luciferase Assay System per well (Promega). RLU were measured as above.

SARS-CoV-2 focus reduction neutralization test (FRNT): HeLa-ACE2 cells were seeded in 12 μL complete DMEM at a density of 2×10³ cells per well. In a dilution plate, pooled mouse serum was diluted in series with a final volume of 12.5 μL. Then 12.5 μL of SARS-CoV-2 virus was added to the dilution plate at a concentration of 1.2×10⁴ pfu/mL.

After 1 hour incubation, the media remaining on the 384-well plate was removed and 25 μL of the virus/serum mixture was added to the 384-well plate. The plate was incubated for 20 hours after which the plate was fixed for 1 hour. Each well was then washed three times with 100 μL of 1×PBS 0.05% tween. 12.5 μL of human polyclonal sera diluted 1:500 in Perm/Wash buffer (BD Biosciences 554723) were added to each well in the plate and incubated at room temperature (RT) for 2 hours. The plate was washed three times and peroxidase goat anti-human Fab (Jackson Scientific) was diluted 1:200 in Perm/Wash buffer then added to the plate and incubated at RT for 2 hours. The plate was then washed three times and 12.5 μL of Perm/Wash buffer was added to the plate then incubated at RT for 5 minutes. The Perm/Wash buffer was removed and TrueBlue peroxidase substrate was immediately added (Sera Care 5510-0030). Sera were tested in triplicate wells. Normal human plasma was used as negative controls for serum screening.

SARS-CoV-2 convalescent plasma samples: COH Institutional Biosafety Committee Protocol 20004 approved the use of SARS-CoV-2 convalescent plasma. Anonymized plasma samples of SARS-CoV-2 convalescent individuals (N=19) were obtained from the University of California, San Diego. Individuals were confirmed to be infected in the previous three to ten weeks by PCR and lateral flow assay. All individuals were symptomatic with mild to moderate-severe symptoms. Serum samples (DS-626-G and DS-626-N, Seracare) purchased before SARS-CoV-2 pandemic were used as a negative control. SARS-CoV-2-specific binding antibodies in plasma samples were measured as described above. Cross-adsorbed goat anti-human IgG (H+L) secondary antibody (A18811, Invitrogen) was used at a dilution of 1:3,000.

T cell analysis: Spleens were harvested and dissociated using a cell mesh following which blood cells were removed using RBC Lysis Buffer (BioLegend). 2.5×10⁶ splenocytes were stimulated with S or N peptide libraries (GenScript, 15mers with 11aa overlap, 1 μg/ml), 0.1% DMSO, or phorbol myristate acetate (PMA)-ionomycin (BD Biosciences) for 1.5 hours at 37° C. Anti-mouse CD28 and CD49d antibodies (1 μg/ml; BioLegend) were added as co-stimulation. Brefeldin A (3 μg/ml; eBioscience) was added, and the cells were incubated for additional 16 hours at 37° C. Cells were fixed using Cytofix buffer (BD Biosciences) and surface staining was performed using fluorescein isothiocyanate (FITC)-conjugated anti-mouse CD3 (Clone 17A2, 555274, BD), BV650 anti-mouse CD8a (Clone 53-6.7, 563234, BD). Following cell permeabilization using Cytoperm buffer (BD Biosciences), ICS was performed using allophycocyanin (APC)-conjugated anti-mouse IFN-γ (Clone XMG1.2, 554413, BD), phycoerythrin (PE)-conjugated anti-mouse TNF-α (Clone MP6-XT22, 554419, BD), and PE-CF594 anti-mouse IL-2 (BD Biosciences (Clone JES6-5H4, 562483, BD). In experiments testing double recombinants SARS-CoV2 vectors IL-2 antibody was not included and PE-CF594 anti-mouse IL-4 (clone 11B11, 562450, BD) and BV421 rat anti mouse IL-10 (clone JES5-16E3, 563276, BD) were added. Events were acquired using a BD FACSCelesta flow cytometer (2×10⁵ cells/tube). Analysis was performed using FlowJo. Antigen specific T cells were identified by gating on size (FSC vs SSC), doublet negative (FSC-H vs FSC-A), CD3+, CD8+/CD4+. Cytokine positive responses are presented after subtraction of the background response detected in the corresponding unstimulated sample (media added with Brefeldin A one hour after beginning of mock stimulation) of each individual mouse sample. Polyfunctional T-cells analysis was performed by applying FlowJo Boolean combination gating.

Cytokine ELISA: Splenocytes (1×10⁶) from immunized mice were incubated in v-bottom wells in the presence of 2 μg/ml S or N peptide pools, or without stimulus in a volume of 200 μl. 48 hours later, plates were centrifuged 2000 RPM for 10 minutes and cell supernatant was collected and stored at −80° C. Mouse TNF-alpha (MTA00B), Quantikine ELISA kit (R&D systems) was used according to manufacturer's recommendations.

IFNγ ELISpot: T-cell detection by IFNγ ELISpot assay was performed according to the manufacturer's instructions (3321-2A, Mabtech). ELISpot PVDF plates (MSIPS4W10, Millipore) were pre-activated with ethanol and coated with IFNγ-coating antibody. Splenocytes (2×10⁵ peptide-stimulated, 2×10⁴ PMA/lonomycin-stimulated) were added to duplicate wells and incubated overnight with 2 μg/mL peptides. Stimuli included S and N peptide libraries; S1 subunit peptide pools covering peptides 1-86 (pool 1S1) and 87-168 (pool 2S1) of the S library; S2 subunit peptide pool that included peptides 169-316 of the S library; and peptide N26

(MKDLSPRWYFYYLGT, SEQ ID NO: 89)

of the N peptide library. After 24 hours, cells were removed, and IFNγ-detection antibody followed by streptavidin-ALP were added. Spots were developed using BCIP/NBT-plus (3650-10, Mabtech) and analyzed using AID ELISpot reader with AID ELISpot 5.0 iSpot software.

Statistics: Statistical evaluation was pursued using GraphPad Prism (v8.3.0). For evaluation of differences in sMVA and wtMVA plaque area in BHK-21 and CEF cells and differences in sMVA and wtMVA host cell range, one-way ANOVA followed by Tukey's and Dunnet's multiple comparison tests were used, respectively. For sMVA and wtMVA growth kinetic analysis, mixed-effects model with the Geisser-Greenhouse correction, followed by Tukey's multiple comparisons test were applied. For ELISAs, one-way ANOVA and Tukey's multiple comparison tests were used to calculate differences in endpoint titers and group means between groups. For IgG2a/IgG1 ratio analysis, one-way ANOVA with Dunnett's multiple comparison test was used to compare the IgG2a/IgG1 ratio measured in each group to a ratio of 1. Pearson correlation analysis was performed to calculate the correlation coefficient r and its significance. For T cell response analysis, one-way ANOVA followed by Dunnett's multiple comparisons test with a single pooled variance was used to compare the mean of each group. For ELISpot analysis, two-way ANOVA with Dunnett's multiple comparison test was applied.

### Example 1: Construction of sMVA

To develop the three-plasmid system of the sMVA vaccine platform, three unique synthetic sub-genomic MVA fragments (sMVA F1-F3) were designed based on the MVA genome sequence published by Antoine et al.⁴, which is about 178 kbp in length and contains about 9.6 kbp inverted terminal repeats (ITRs) (FIG. 1A). The three fragments were designed as follows: sMVA F1 comprises about 60 kbp of the left part of the MVA genome, including the left ITR sequences; sMVA F2 contains about 60 kbp of the central part of the MVA genome; and sMVA F3 contains about 60 kbp of the right part of the MVA genome, including the right ITR sequences (FIG. 1B). sMVA F1 and F2 as well as sMVA F2 and F3 were designed to share about 3 kb overlapping homologous sequences to promote recombination of the three sMVA fragments (FIG. 1B). In addition, a duplex copy of the 165-nucleotide long MVA terminal hairpin loop (HL) flanked by concatemeric resolution (CR) sequences was added to both ends of each of the three sMVA fragments (FIG. 1C). Such CR/HL/CR sequence arrangements are formed at the genomic junctions in poxvirus DNA replication intermediates and essential for genome resolution and packaging²⁷⁻³¹. When circular DNA plasmids containing these CR/HL/CR sequence arrangements are transfected into helper virus-infected cells they spontaneously resolve into linear minichromosomes with intact terminal HL sequences^{28,29,32}. The three sMVA fragments designed as shown in FIGS. 1B-1C, when co-transfected as circular DNA plasmids into helper virus infected cells, can resolve into linear minichromosomes, recombine with each other via the shared homologous sequences, and are ultimately packaged as full-length MVA genomes. All three sMVA fragments were cloned in E. coli as bacterial artificial chromosome (BAC) clones.

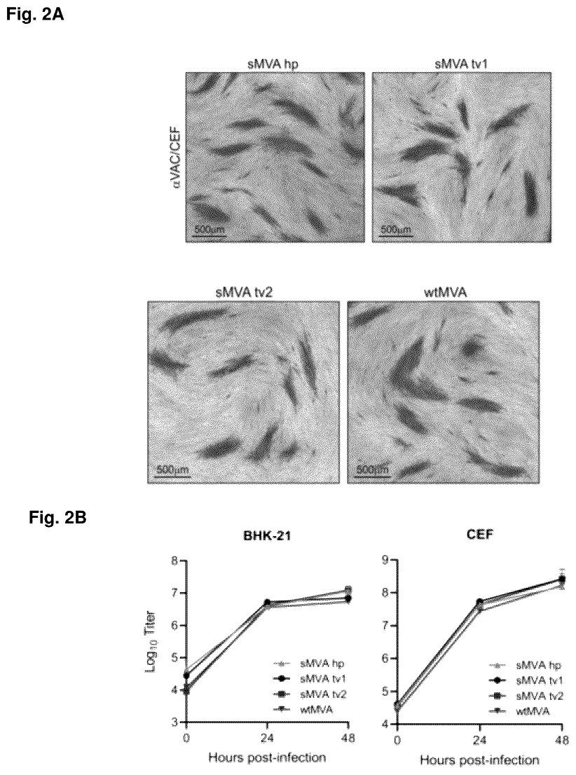

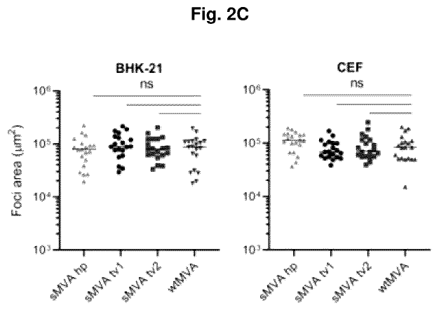

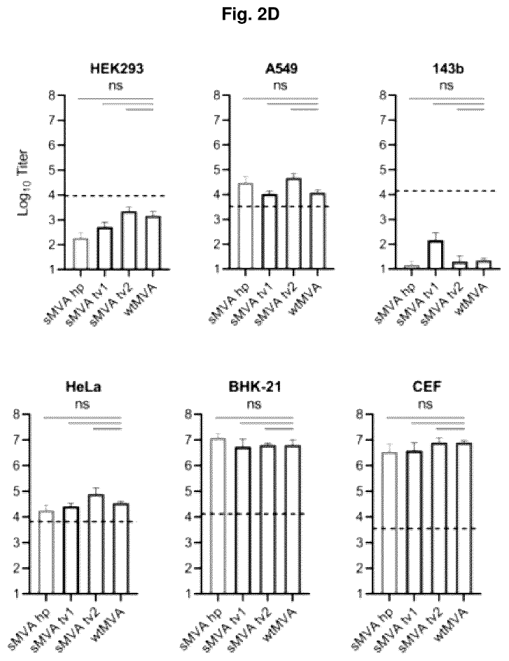

Using a previously employed procedure to rescue MVA from a BAC^{8,9,33}, sMVA virus was reconstituted with Fowl pox (FPV) as a helper virus upon co-transfection of the three DNA plasmids into BHK-21 cells (FIG. 1D), which are non-permissive for FPV³⁴. Two different FPV strains (HP1.441 and TROVAC)^{35,36} were used to promote sMVA virus reconstitution (FIG. 2A). The purified sMVA virus was produced following virus propagation in CEF, which are commonly used for MVA vaccine production. The virus titers achieved with reconstituted sMVA virus were similar to virus titers achieved with “wild-type” MVA (wtMVA) (Table 1).

TABLE 1

| Construct | Insert (insertion site) | Titer* |
|---|---|---|
| sMVA hp | None | 6.8 × 10⁹ PFU/ml |
| sMVA tv1 | None | 4.1 × 10⁹ PFU/ml |
| sMVA tv2 | None | 2.3 × 10⁹ PFU/ml |
| wtMVA | None | 4.1 × 10⁹ PFU/ml |
| sMVA-S tv | Spike (Del3) | 4.3 × 10⁹ PFU/ml |
| sMVA-N tv | Nucleocapsid (Del3) | 1.0 × 10¹⁰ PFU/ml |
| sMVA-S/N hp | Spike (G1L), Nucleocapsid (Del3) | 8.8 × 10⁹ PFU/ml |
| sMVA-N/S hp | Nucleocapsid (Del2), Spike (Del3) | 2.3 × 10⁹ PFU/ml |
| sMVA-S/N tv | Spike (G1L), Nucleocapsid (Del3) | 8.8 × 10⁹ PFU/ml |
| sMVA-N/S tv | Nucleocapsid (Del2), Spike (Del3) | 8.4 × 10⁹ PFU/ml |

*Stocks were produced on CEF following infection (MOI 0.02) of 30 × 15 cm dishes

### Example 2: In Vitro Characterization of sMVA

To characterize the viral DNA of sMVA, DNA extracts from sMVA and wtMVA-infected CEF were compared for several MVA genome positions by PCR.¹⁵ Similar PCR results were obtained with sMVA and wtMVA for all evaluated genome positions (FIG. 1E), including the F1/F2 and F2/F3 recombination sites, indicating efficient recombination of the three sMVA fragments. Additional PCR analysis indicated the absence of any BAC vector sequences in sMVA viral DNA (FIG. 1E), suggesting spontaneous and efficient removal of the bacterial vector elements upon sMVA virus reconstitution. Comparison of viral DNA from purified sMVA and wtMVA virus by restriction enzyme digestion revealed similar genome pattern between sMVA and wtMVA (FIG. 1F). Sequencing analysis of the sMVA viral DNA confirmed the MVA genome sequence at several positions, including the F1/F2 and F2/F3 recombination sites. Furthermore, whole genome sequencing of one of the sMVA virus isolates reconstituted with FPV TROVAC confirmed the assembly of the reference MVA genome sequence and absence of vector-specific sequences in viral DNA originating from the reconstituted sMVA virus.

To characterize the replication properties of sMVA, growth kinetics of sMVA and wtMVA were compared on BHK-21 and CEF cells, two cell types known to support productive MVA replication⁶. This analysis revealed similar growth kinetics of sMVA and wtMVA on both BHK-21 and CEF cells (FIG. 2B). In addition, similar areas of viral foci were determined in BHK-21 and CEF cell monolayers infected with sMVA or wtMVA (FIG. 2C), suggesting similar capacity of sMVA and wtMVA to spread in MVA permissive cells. Compared to the productive replication of sMVA and wtMVA in BHK-21 and CEF cells⁶, only limited virus production was observed with sMVA or wtMVA following infection of various human cell lines (FIG. 2D). These results are consistent with the severely restricted replication properties of MVA and show that the sMVA virus can efficiently propagate in BHK-21 and CEF cells, while it is unable to efficiently propagate in human cells.

### Example 3: In Vivo Immunogenicity of sMVA

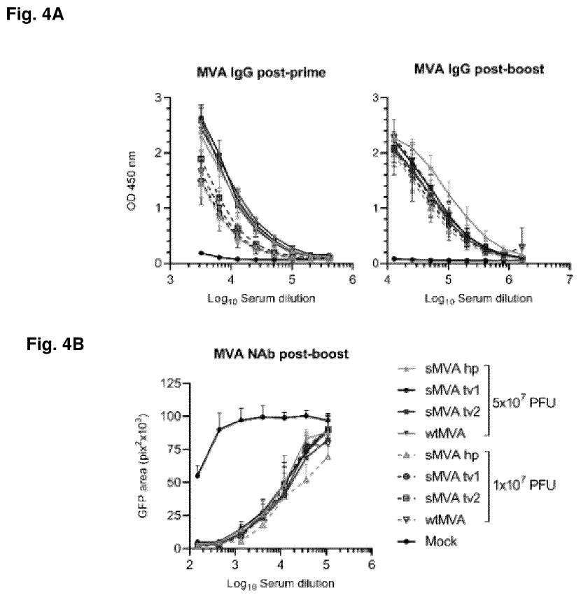

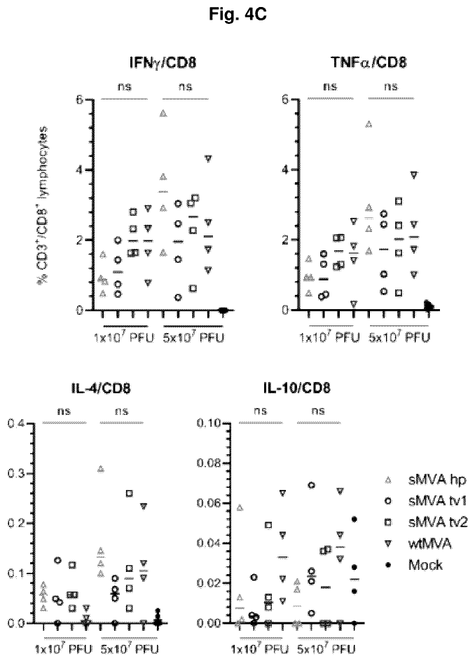

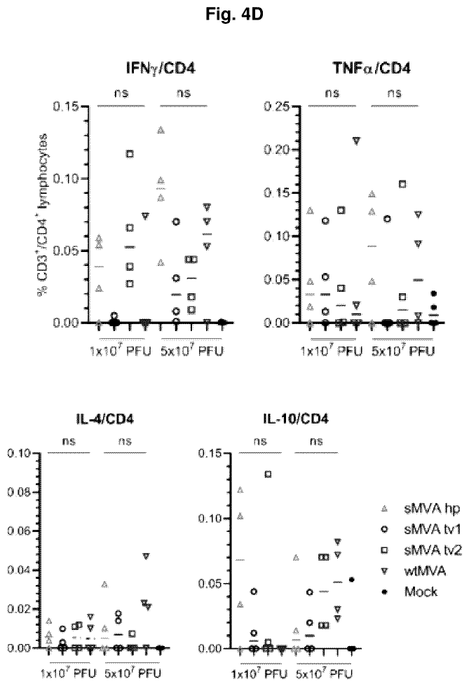

To characterize sMVA in vivo, the immunogenicity of sMVA and wtMVA was compared in C57BL/6 mice following two immunizations at high or low dose. MVA-specific binding antibodies stimulated by sMVA and wtMVA after the first and second immunization were comparable (FIGS. 3A, 4A). While the antibody levels in the high dose vaccine groups exceeded those of the low dose vaccine groups after the first immunization, similar antibody levels in the high and low dose vaccine groups were observed after the second immunization. In addition, no significant differences were detected in the levels of MVA-specific NAb responses induced by sMVA and wtMVA after the second immunization (FIGS. 3B, 4B). MVA-specific T cell responses determined after the booster immunization by ex vivo antigen stimulation using immunodominant peptides³⁵ revealed similar MVA-specific T cell levels in mice receiving sMVA or wtMVA (FIGS. 3C-3D and 4C-4D). These results indicate that the sMVA virus has a similar capacity as wtMVA in inducing MVA-specific humoral and cellular immunity in mice.

### Example 4: Construction of sMVA SARS-CoV-2 Vaccine Vectors

Using highly efficient BAC recombination techniques in E. coli, full-length SARS-CoV-2 S and N antigen sequences were inserted into commonly used MVA insertions sites located at different positions within the three sMVA fragments. Combinations of modified and unmodified sMVA fragments were subsequently co-transfected into FPV-infected BHK-21 cells to reconstitute sMVA SARS-CoV-2 (sMVA-CoV2) vectors expressing the S and N antigen sequences alone or combined (FIGS. 5A and 5B). In the single recombinant vectors encoding S or N alone, termed sMVA-S and sMVA-N, respectively, the antigen sequences were inserted into the Deletion (Del3) site (FIGS. 1B and 5B) 5. In the double recombinant vectors encoding both S and N, termed sMVA-N/S and sMVA-S/N, the antigen sequences were inserted into Del3 and the Deletion 2 (Del2) site (sMVA-N/S), or they were inserted into Del3 and the intergenic region between 069R and 070L (IGR69/70) (sMVA-S/N) (FIGS. 1B and 5B)^{5,38}. All antigen sequences were inserted into the sMVA-CoV2 vectors together with mH5 promoter to promote antigen expression during early and late phase of MVA replication^{39,40}. sMVA-CoV-2 vaccine vectors were reconstituted with FPV strain HP1.441 or TROVAC. The purified virus of the sMVA-CoV2 vectors produced using CEF reached titers that were similar to those achieved with sMVA or wtMVA (Table 1). PCR and sequencing analysis of the Del2, Del3, and IGR69/70 MVA insertion sites confirmed the integrity and insertion of the SARS-CoV-2 antigen sequences in all sMVA-CoV2 vaccine vectors (FIG. 5C). Moreover, whole-genome sequencing of all double-recombinant sMVA-CoV2 vaccine vectors—reconstituted either with FPV strain TROVAC or HP1.441—verified the reference sequences of these vaccine constructs deposited in NCBI and confirmed the SARS-CoV-2 antigen sequences at the insertion sites, the identity of the MVA genome, and removal of the BAC vector sequences.

### Example 5: In Vitro Characterization of sMVA-CoV2 Vaccine Vectors

To characterize S and N antigen expression by the sMVA-CoV2 vectors, BHK-21 cells infected with the sMVA-CoV2 vectors were evaluated by Immunoblot using S and N-specific antibodies. This analysis confirmed the expression of the S or N antigen alone by the single recombinant vaccine vectors sMVA-S and sMVA-N, while the expression of both the S and the N antigen was confirmed for the double recombinant vectors sMVA-N/S and sMVA-S/N (FIG. 5D).

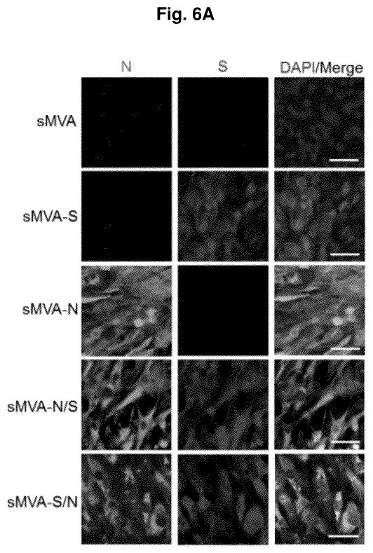

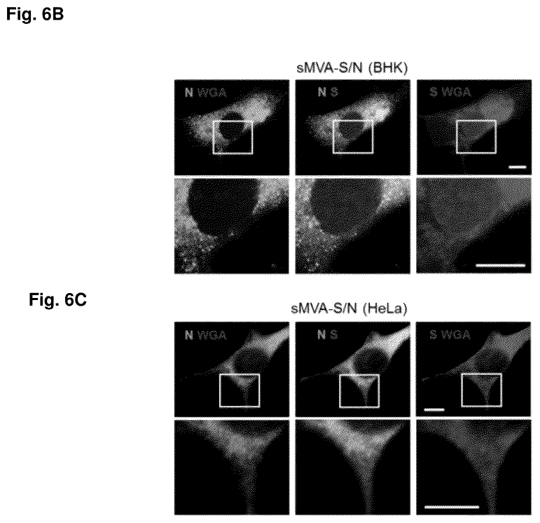

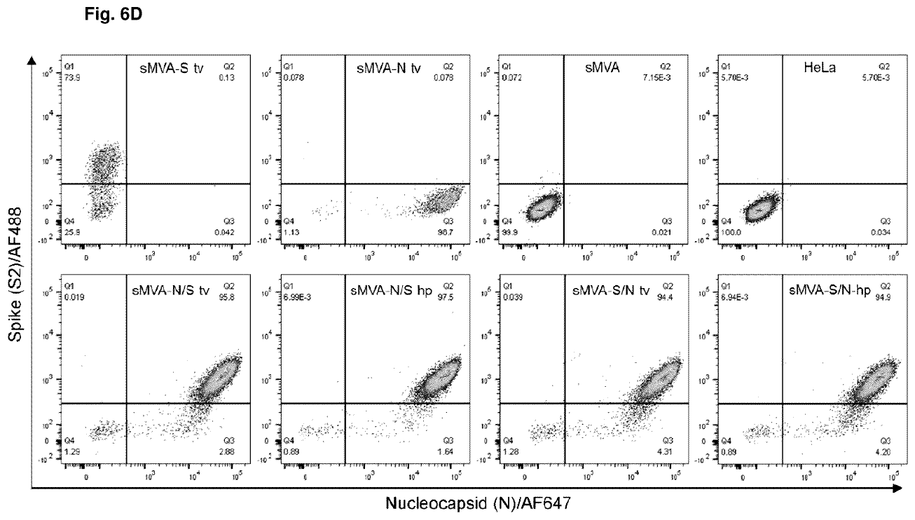

Further characterization of the antigen expression by the sMVA-CoV2 vectors in Hela cells using cell surface and intracellular flow cytometry (FC) staining confirmed single and dual S and N antigen expression by the single and double recombinant vaccine vectors. Staining with S-specific antibodies revealed abundant cell surface and intracellular antigen expression by all vectors encoding the S antigen (sMVA-S, sMVA-N/S, sMVA-S/N) (FIG. 5E). In contrast, staining with anti-N antibody revealed predominantly intracellular antigen expression by all vectors encoding the N antigen (sMVA-N, sMVA-N/S, sMVA-S/N) (FIG. 5E), although cell surface staining was also observed to a minor extent. S and N antigen expression by the sMVA-CoV2 vectors was also investigated by immunofluorescence. This analysis confirmed co-expression of the S and N antigens by the double recombinant vaccine vectors and indicated efficient cell surface and intracellular expression of the S antigen, whereas the expression of the N antigen was predominantly observed intracellular (FIGS. 6A-6C). Furthermore, immunofluorescence imaging in addition to intracellular flow cytometry by dual antibody staining demonstrated co-expression of the S and N antigens within the same cell by the double recombinant sMVA-CoV2 vectors (FIGS. 6A-6D). These results demonstrate efficient antigen expression by the single and double recombinant sMVA-CoV2 vectors.

### Example 6: In Vivo Immunogenicity of sMVA-CoV2 Vectors

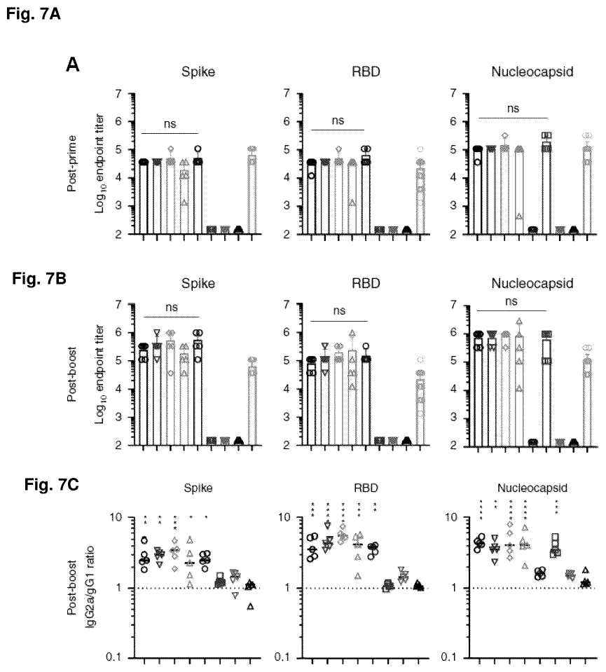

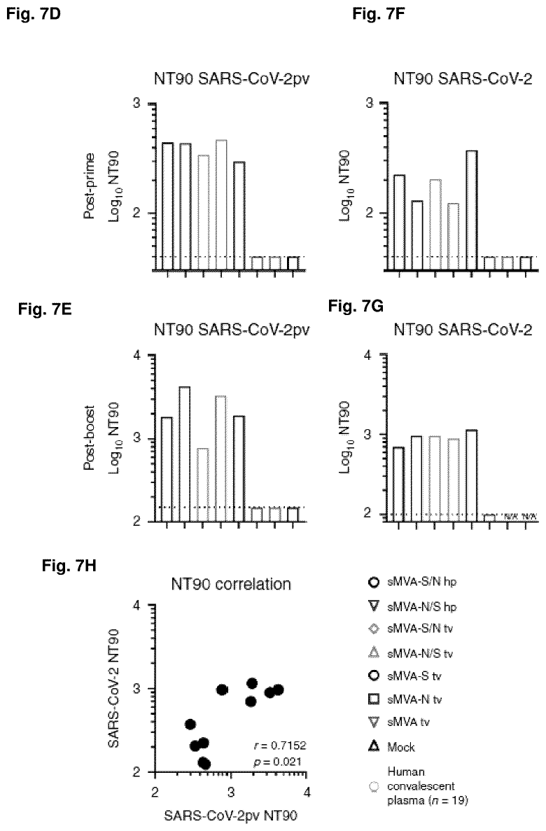

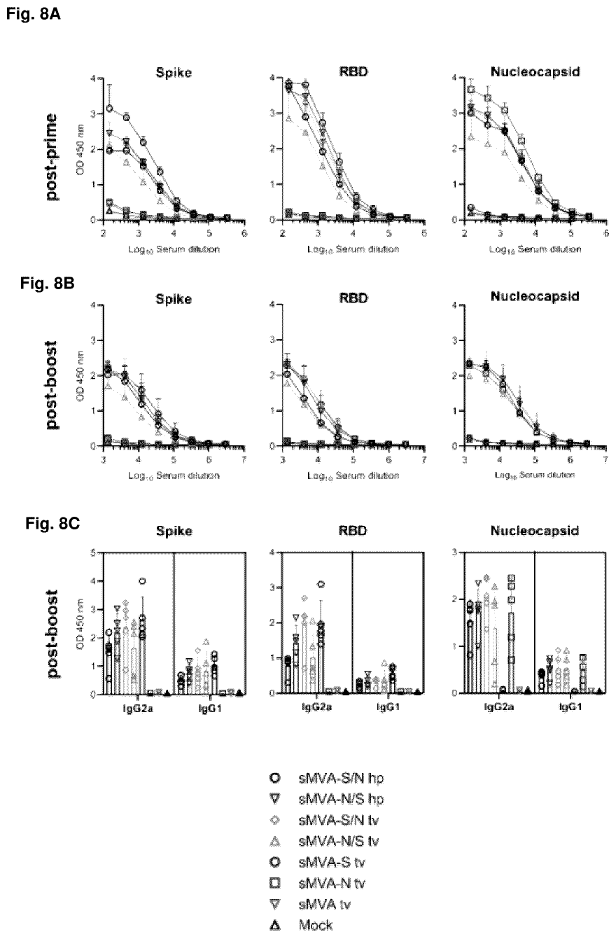

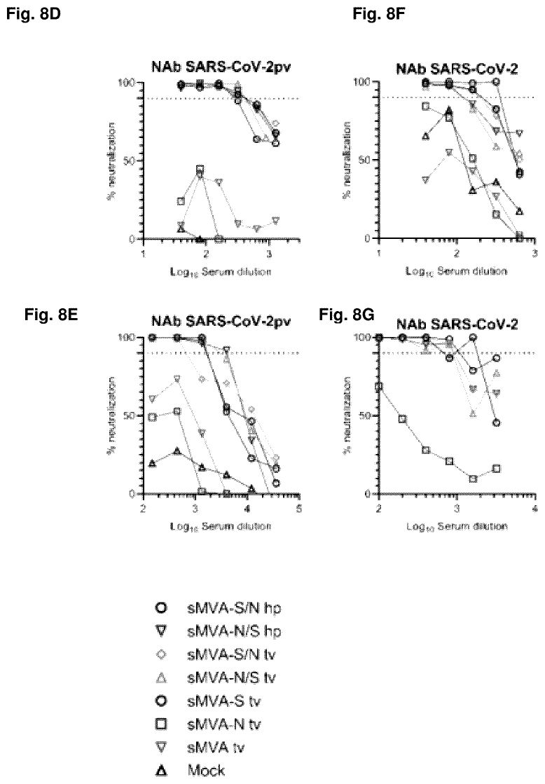

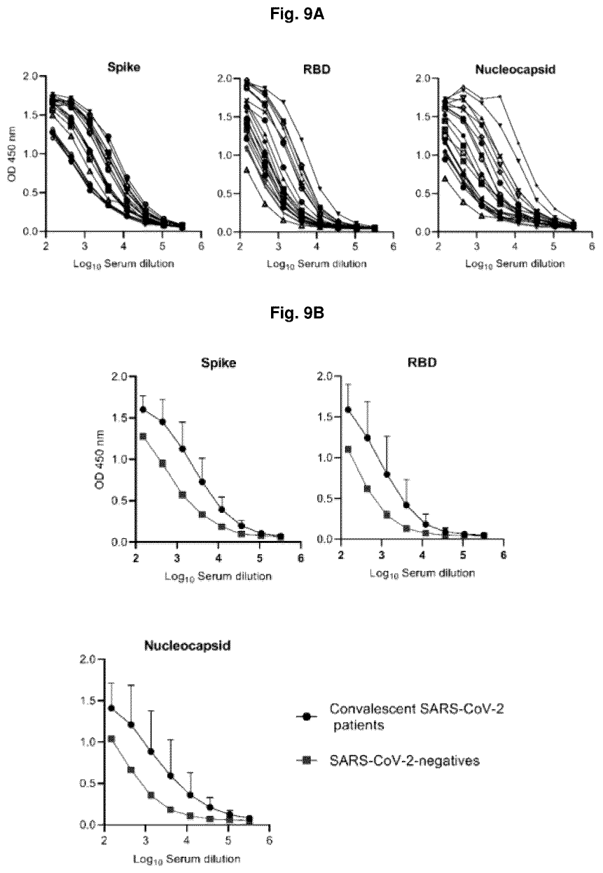

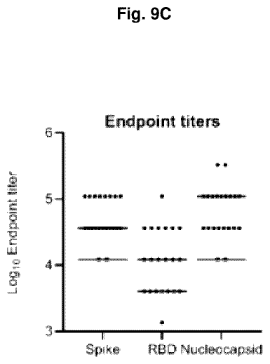

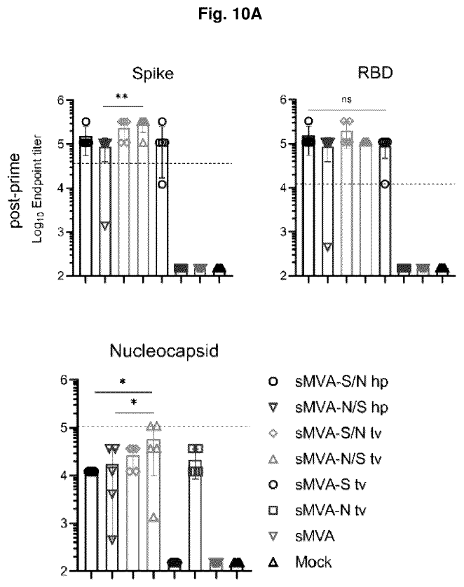

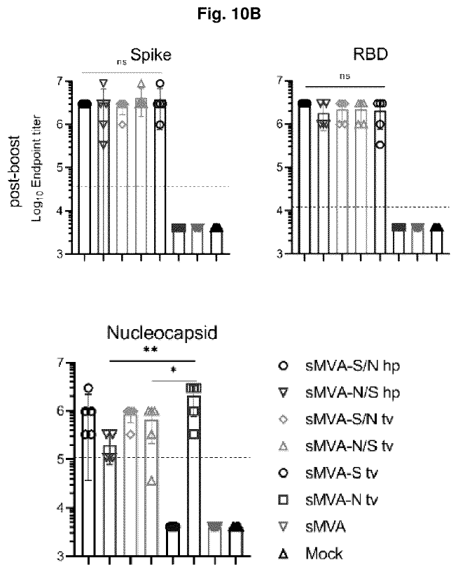

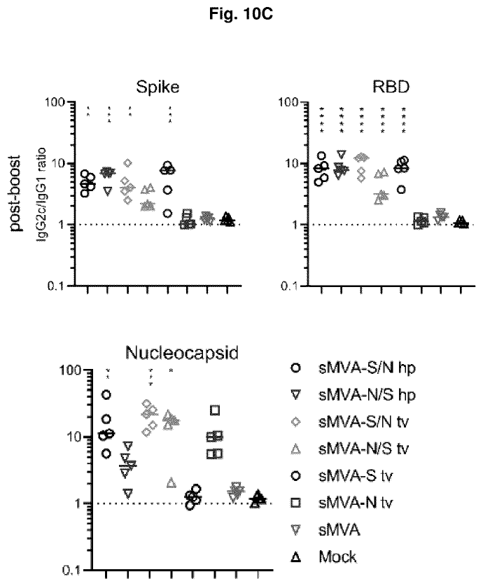

To determine the immunogenicity of the sMVA-vectored S and N antigens alone or combined, SARS-CoV-2-specific humoral and cellular immune responses were evaluated in Balb/c mice by two immunizations with the single or double recombinant vaccine vectors. High-titer antigen-specific binding antibodies were detected in all vaccine groups after the first immunization, and an increase in these responses was observed after the booster immunization (FIGS. 7A-7B and 8A-8B). While the single recombinant vectors induced binding antibodies only against the S or N antigen, the double recombinant vectors induced binding antibodies against both the S and N antigens. In addition, all sMVA-CoV2 vectors encoding the S antigen (sMVA-S, sMVA-S/N, sMVA-N/S) stimulated high-titer binding antibodies against the S receptor binding domain (RBD), which is considered the primary target of NAb^{22,24}. Antigen-specific binding antibody titers between the single and double recombinant vaccine groups were comparable. Notably, SARS-CoV-2 antigen-specific binding antibody responses stimulated by the sMVA-CoV2 vaccine vectors in mice exceeded SARS-CoV-2 S-, RBD-, and N-specific binding antibody responses measured in human convalescent immune sera (FIGS. 7A-7B, and 9). Similar binding antibody responses to those induced by sMVA-CoV2 vectors in Balb/c mice were elicited by the vaccine vectors in C57BL/6 mice (FIG. 10). Analysis of the IgG2a/IgG1 isotype ratio of the binding antibodies revealed Th-1-biased immune responses skewed toward IgG2a independently of the investigated vaccine group or antigen (FIGS. 7C and 8C).

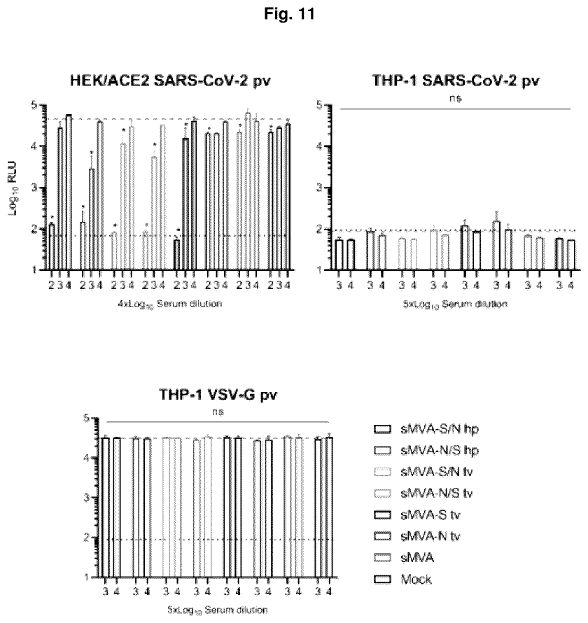

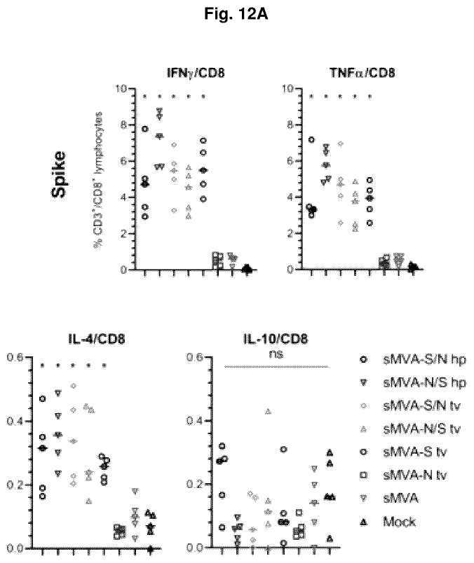

Potent SARS-CoV-2-specific NAb responses as assayed using pseudovirus were detected after the first immunization in all vaccine groups receiving the vectors encoding the S antigen (sMVA-S, sMVA-S/N, sMVA-N/S), and these NAb responses increased after the booster immunization (FIGS. 7D-7E and 8D-8E). Similar potent NAb responses as measured using pseudovirus were observed in the vaccine groups using infectious SARS-CoV-2 virus (FIGS. 7F-7G and 8F-8G). The immune sera for potential antibody-dependent enhancement of infection (ADE) were evaluated using THP-1 monocytes. These cells do not express the ACE2 receptor, but express Fcγ receptor II, which is considered the predominant mediator of ADE in SARS-COV infection⁴¹. THP-1 monocyte infection by SARS-CoV-2 pseudovirus was not promoted by the immune sera of any of the vaccine groups even at sub-neutralizing antibody concentrations (FIG. 11), suggesting absence of Fc-mediated ADE by antibodies induced by the vaccine vectors.

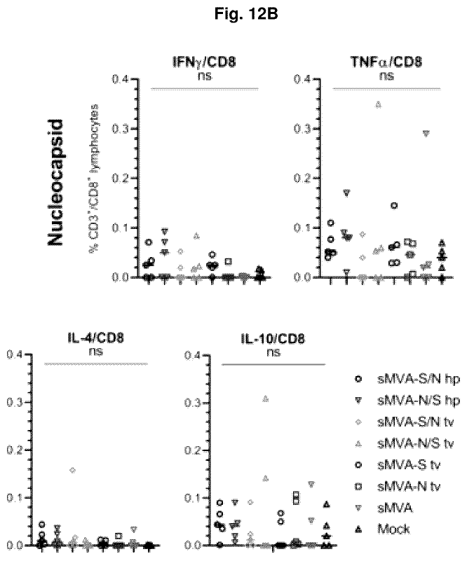

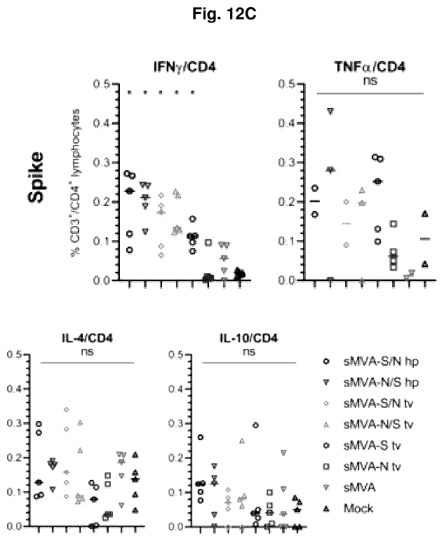

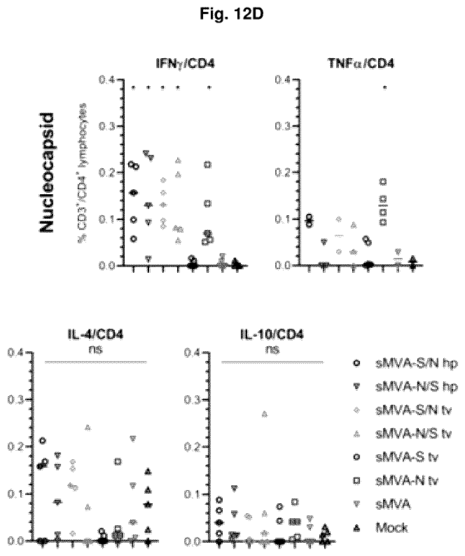

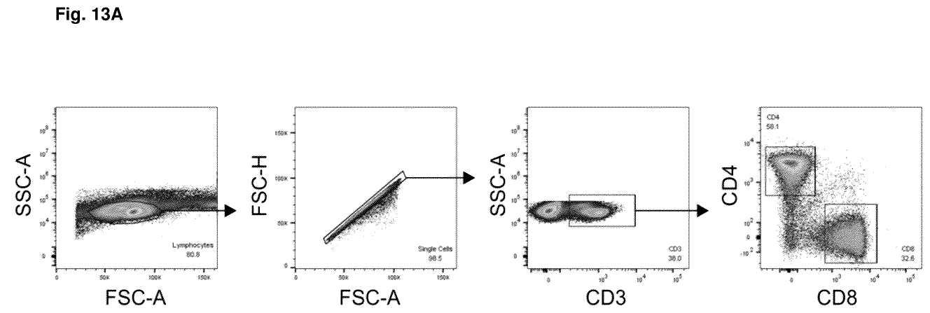

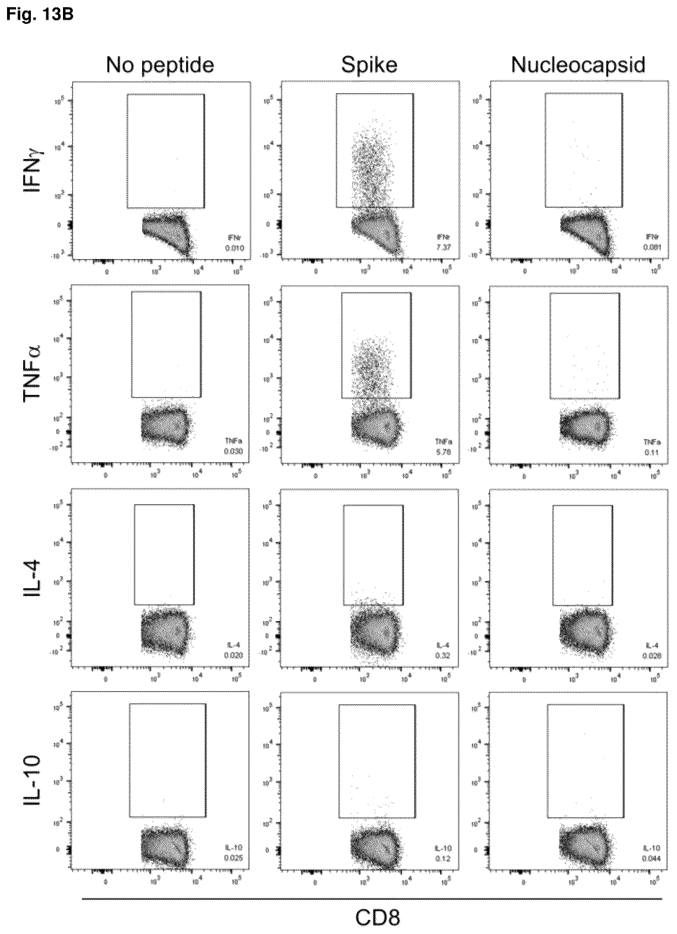

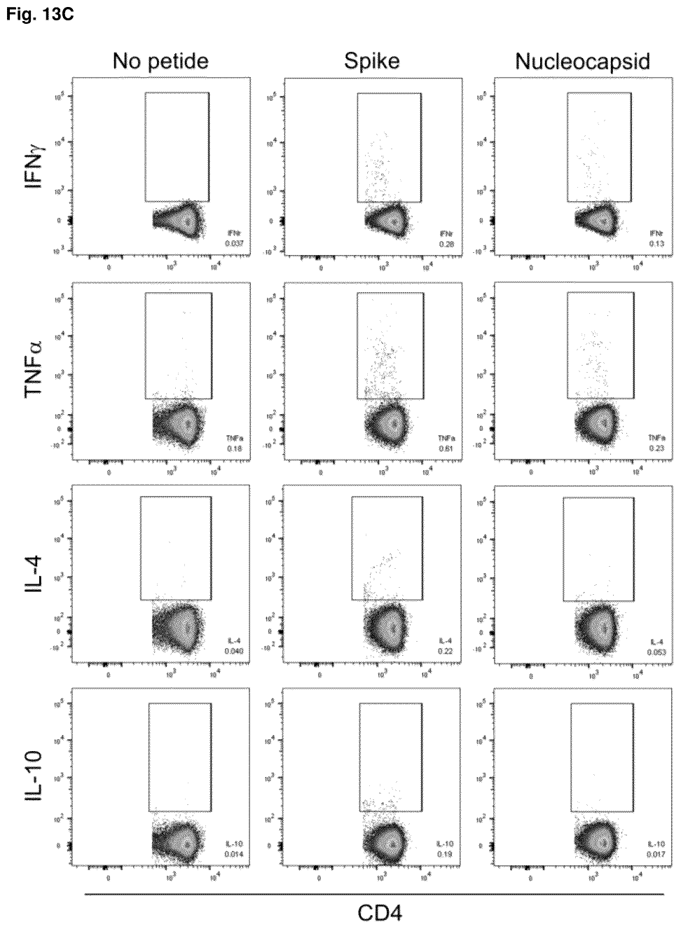

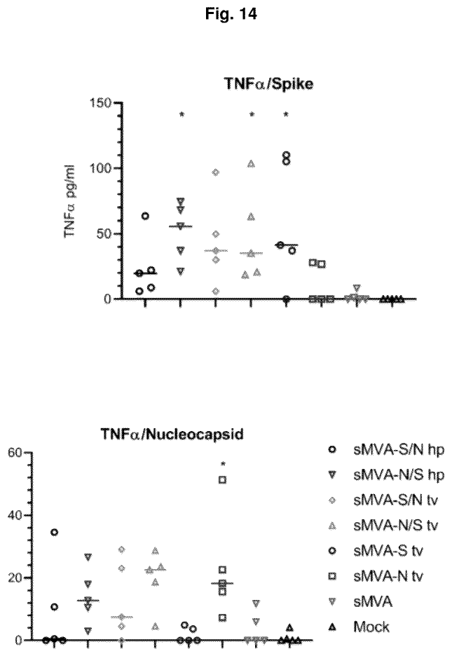

SARS-CoV-2-specific T cells evaluated after the second immunization by ex vivo antigen stimulation revealed both S- and N-specific T cell responses in the vaccine groups receiving the double recombinant vectors sMVA-S/N and sMVA-N/S. In contrast, mice receiving the single recombinant vectors sMVA-N or sMVA-S developed T cell responses only against either the N or S antigen (FIGS. 12A-12D, 13, and 14). High levels of cytokine-secreting (IFNγ, TNFα and IL-4) S-specific CD8+ T cells were measured in all vaccine groups immunized with the S-encoding sMVA-CoV2 vectors (FIG. 12A). S-specific CD4+ T-cells mostly produced Th1 cytokines (IFNγ and TNFα), while production of Th2 cytokines (IL-4 and IL-10) did not increase following antigen stimulation (FIGS. 12C, 14), indicating a Th1-biased response. While activated N-specific CD8+ T cells were not detected at significant frequency (FIG. 12B), N-specific IFNγ and to some degree TNFα-secreting CD4+ T cells were measured in all animals vaccinated with the single and double recombinant vectors encoding N (FIGS. 12D and 14). No significant differences were observed in the T cell levels of the single and double recombinant vaccine groups.

Stimulation of SARS-CoV-2-specific immune responses by both the S and N antigens was also evaluated in mice by co-immunization using the single recombinant vectors sMVA-S and sMVA-N at different doses. This study revealed similar SARS-CoV-2 antigen-specific humoral and cellular immune responses in vaccine groups receiving sMVA-S and sMVA-N alone or in combination (FIGS. 15 and 16). Altogether these results indicate that the sMVA-vectored S and N antigens when expressed alone or combined using a single vector or two separate vectors can stimulate potent SARS-CoV-2-specific humoral and cellular immune responses in mice.

### Example 7: In Vivo Immunogenicity of COH04S1 in Mice

Mice immunized with sMVA vaccine, COH04S1, either once or twice, demonstrated high titers of binding antibodies, neutralizing antibodies and T cell reactivity. These results suggest that COH04S1 is highly immunogenic in mice. See Table 2 below. NT50/90 is the dilution of the (antibody-containing) serum still showing 50/90% neutralization of infection. In combination with extensive prior safety and clinical experience of MVA and as a platform to address future variants of coronaviruses, the vaccine disclosed herein has potentially significant clinical use.

TABLE 2
Summary of mouse immunogenicity elicited by COH04S1

| 1 | Total SARS-CoV-2 | Endpoint titer: | Endpoint titer: |
|---|---|---|---|
| | binding antibodies | approx. 10⁵ | approx. 10⁶ |
| 2.1 | T-cell immune response | Both for Spike and Nucleocapsid: CD4⁺ and | |
| 2.2 | | CD8⁺ T cells show Th1 polarization (indicative of | |
| | | cell-mediated immunity against pathogens), | |
| | | predominantly releasing TNFα and IFNγ | |
| 3 | Neutralizing | NT50*: approx. 10³ | NT50: approx. 10⁴ |
| | antibodies | NT90: 2.2-2.5*10² | NT90: 3-4*10³ |
| 4 | Antibody-dependent | None found | None found |
| | enhancement of infection | | |

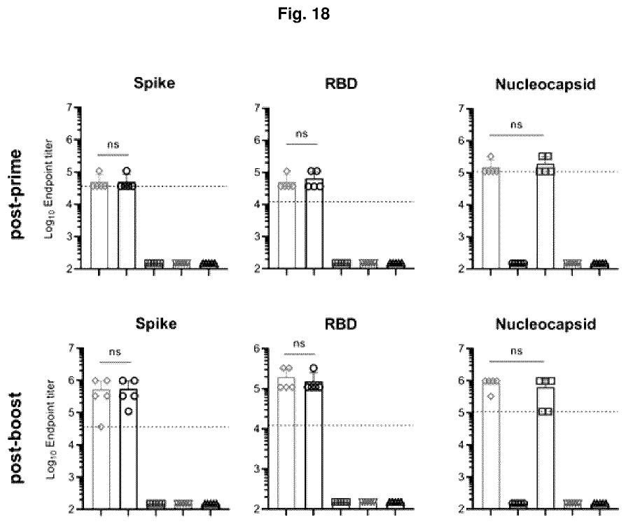

FIG. 18 demonstrates high titers of total binding antibodies directed against Spike (S), Receptor Binding Domain (RBD) and Nucleocapsid (N) antigen shown after first (top panels) and second (bottom panels) immunization with C46 expressing both S and N antigens. The antibody titers compared favorably with antibody titers in convalescent human sera (dotted lines). S-specific antibody responses effectively bound to mutated S (D614G) antigen (data not shown).

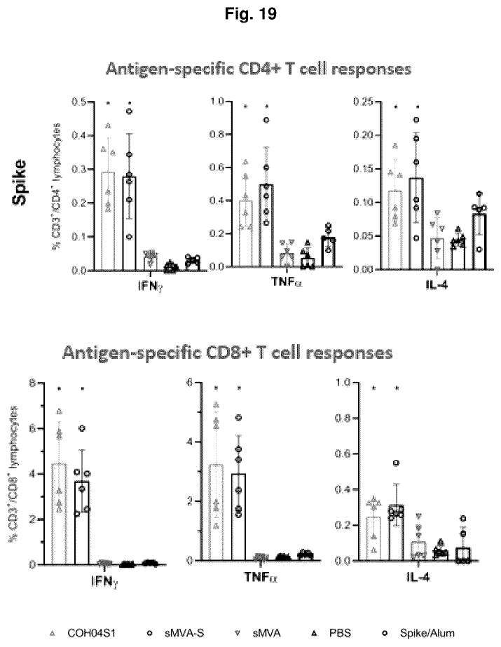

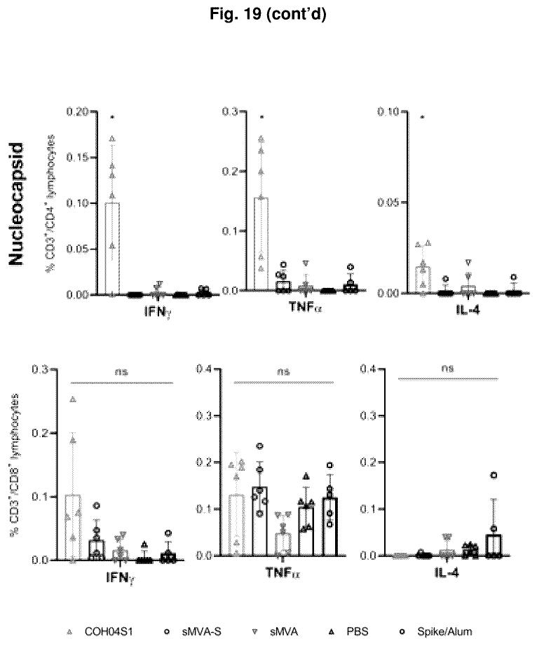

FIG. 19 demonstrates antigen-specific CD4+ (left side) and CD8+ (right side) T cell responses in mice vaccinated with dual antigen construct sMVA-N/S (C35) as well as single- and no-antigen and control. IFNγ and TNFα cytokine production demonstrates robust anti-Spike Th1 cytokine response. Absence of response to IL-4 (and IL-10, data not shown) points to lack of Th2 response.

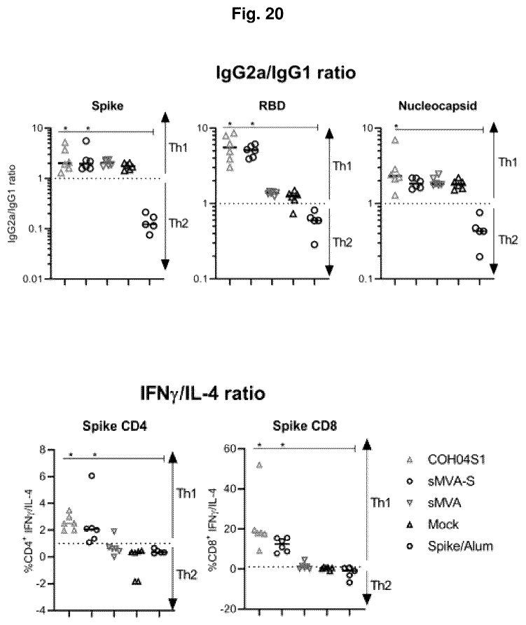

FIG. 20 shows the ratios of IGg2a to IgG1 and IFNγ to IL-4 secretion, demonstrating that vaccination with sMVA-N/S (C35) resulted predominantly in a humoral and cellular Th1 response (which is instrumental in cell-mediated immunity against pathogens), not a Th2 response. Vaccination with Spike antigen mixed with Alum adjuvant (prototype adjuvant for induction of a Th2 response) is shown as control on the right in each panel.

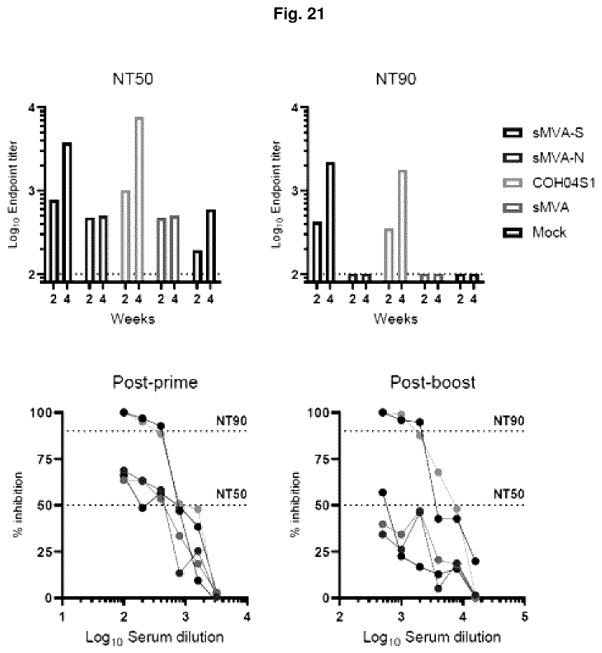

FIG. 21 shows antibodies in mouse serum after one (“post-prime”) or two (“post-boost”) immunizations with dual-antigen COH04S1, single-antigen and empty vectors as well as mock control demonstrate effective development of neutralizing antibodies when using vectors expressing the Spike antigen. sMVA vaccine expressing only Spike antigen is shown in blue (sMVA-S), sMVA vaccine expressing only Nucleocapsid antigen is shown in red (sMVA-N), sMVA vaccine expressing both Spike (S) and Nucleocapsid (N) antigens is shown in green (COH04S1), sMVA vector without SARS-CoV-2 insert is shown in brown (sMVA), and the mock vaccination is shown in navy (mock). Neutralizing antibodies were measured using live SARS-CoV-2 to infect HeLa-Ace2 cells.

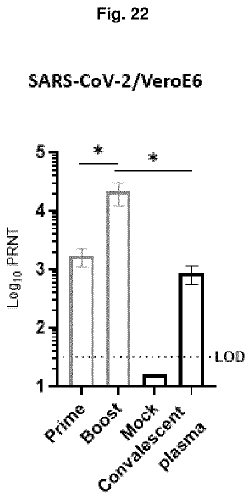

FIG. 22 shows that COH04S1 elicited potent SARS-CoV-2-specific neutralizing antibodies (Nab) in mice (using live SARS-CoV-2 virus). NAbs effectively blocked infection of SARS-CoV-2 infectious virus. The NAb tittered 1-2 orders of magnitude above titers that are considered protective for SARS-CoV-2 infection.

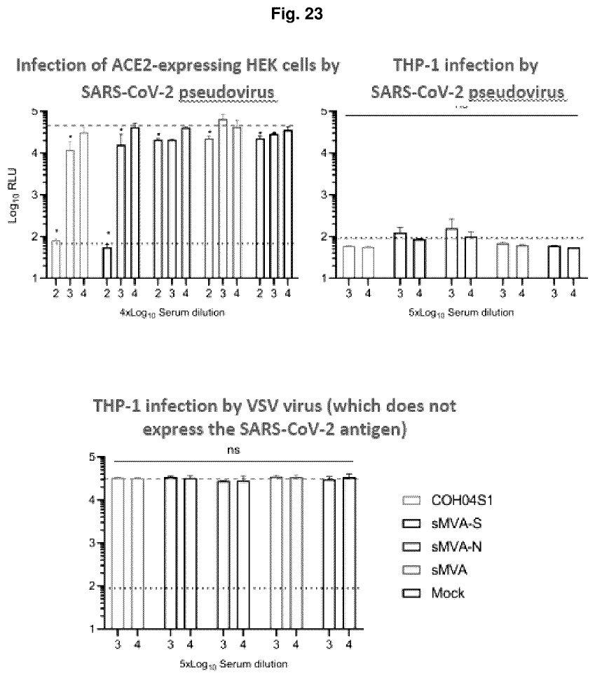

FIG. 23 shows that C46 did not demonstrate evidence of characteristic synonymous with antibody-dependent enhancement (ADE) of infection. The left panel shows HEK cells expressing human ACE2 protein. Pseudovirus infection was successfully prevented by antibodies generated after infection of mice with C46 as well as a single S-expressing vector. No inhibition of infection by controls and no ADE (this would show as RLU units above upper dotted line) by serum antibodies from mice treated with any vector was observed. This panel was the positive control showing that the vaccine was working. The middle panel shows the THP-1 monocytic cell line, not expressing ACE2 receptor (and therefore not capable of being infected by SARS-CoV-2 virus) but expressing Fc receptors (which are suspected in causing ADE). No infection and no ADE were observed in this experiment (no increase above the dotted line). The right panel shows THP-1 cells, positive control infection with VSV vector. No reduction of this infection in the presence of antibodies from treated mice, and no ADE effect observed in the presence of these antibodies either (RLU did not increase above the upper dotted line). RLU represents relative luciferase units, a measure of the degree of infection in this system.

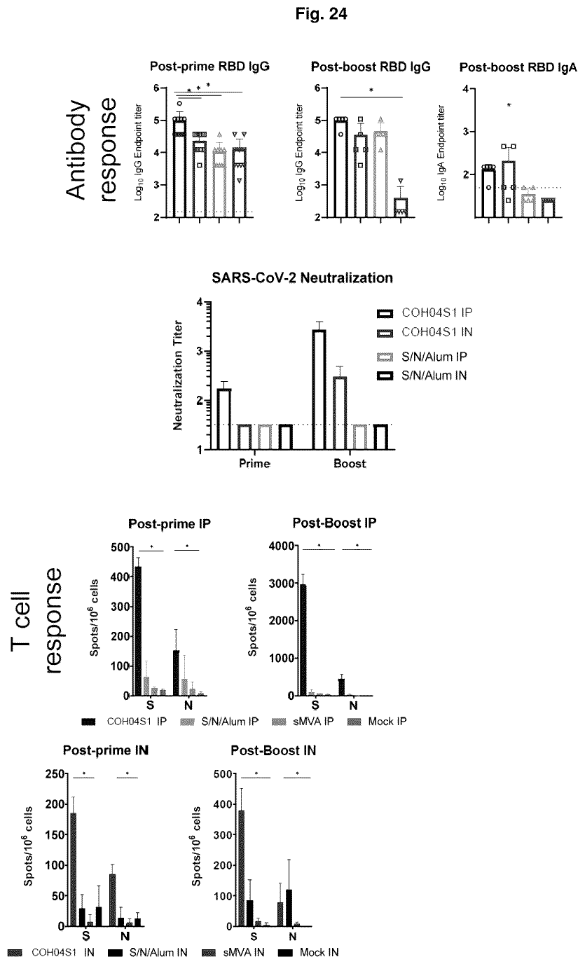

FIG. 24 shows that COH04S1 induced strong humoral and cellular immune responses in mice following intraperitoneal (IP) and intranasal (IN) vaccinations.

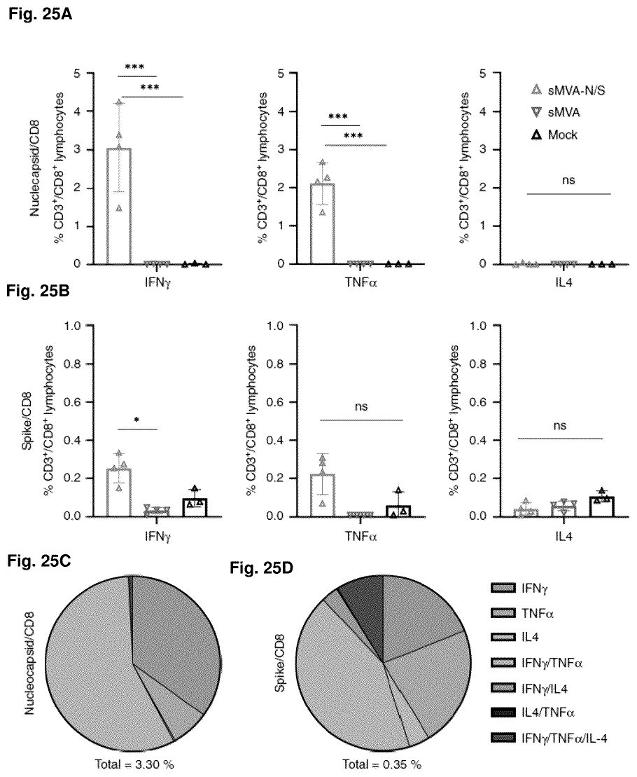

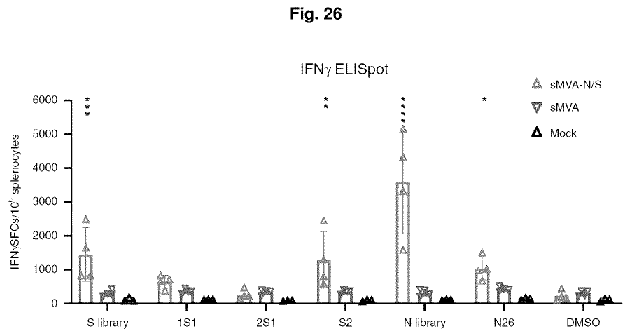

To further assess the immunogenicity of the sMVA-vectored N antigen, the double recombinant vaccine vector sMVA-N/S was evaluated for antigen-specific T-cell stimulation in transgenic C57BL/6 mice expressing the human leukocyte antigen (HLA)-B*0702 (B7). This HLA type has been recently described to present immunodominant N-specific peptides that are frequently recognized in SARS-CoV-2-infected patients. C57BL/6 B7 mice immunized with sMVA-N/S developed high-frequency N-specific CD8+ T cells secreting IFNγ and TNFα that reached over 2-3% of the total CD8+ T-cell population (FIG. 25A). S-specific CD8+ cells secreting IFNγ were also detected at significant levels in sMVA-N/S-immunized C57BL/6 B7 mice, albeit at lower frequency compared to the N-specific T-cell responses (FIG. 25B). N- or S-specific CD8+ T cells secreting IL-4 were not observed in significant levels in sMVA-N/S-immunized animals. Notably, sMVA-N/S-stimulated CD8+ T cells to both the N and S antigens in C57BL/6 B7 mice were largely polyfunctional, with more than half of the N-specific CD8+ T cells secreting IFNγ and TNFα combined (FIGS. 25C and 25D). Further analysis by IFNγ ELISPOT revealed that the S-specific T-cell responses induced by sMVA-N/S in C57BL/6 B7 mice were mostly directed toward epitopes of the S2 domain (FIG. 26). In addition, a significant response was measured in sMVA-N/S-immunized B7 mice following stimulation with an HLA-B*0702 immunodominant N-specific peptide epitope (SPRWYFYYL, SEQ ID NO: 90) that has been shown recently to be recognized by a high proportion of people recovering from COVID-19 disease (FIG. 26). FIG. 26 shows that a major component of the response to Spike antigen is to the S2 domain. The N library is well recognized as it is in humans. A significant component was identified as a previously described N peptide: N26

(SEQ ID NO: 89)
MKDLSPRWYFYYLGT.

Bold and underlined is the epitope that is described in humans: Peng, Y., Mentzer, A. J., Liu, G. et al., Broad and strong memory CD4+ and CD8+ T cells induced by SARS-CoV-2 in UK convalescent individuals following COVID-19. Nat Immunol (2020). doi.org/10.1038/s41590-020-0782-6. These results demonstrate that both the sMVA-vectored N and S antigens are immunogenic in HLA B*0702 transgenic mice, while the CD8+ T-cell response targeting N appears to be immunodominant.

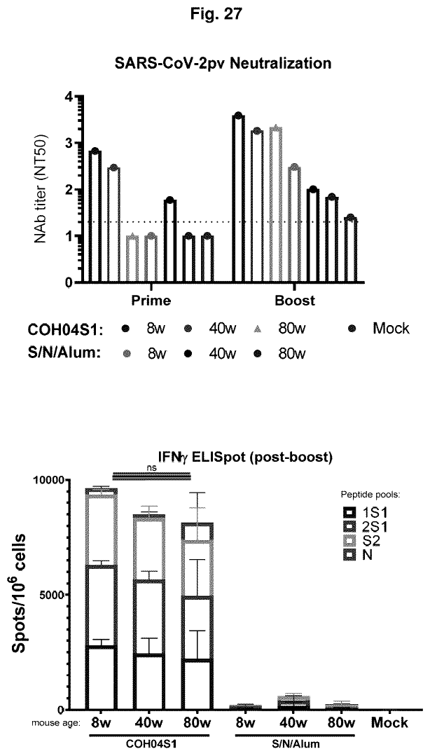

FIG. 27 shows that aged mice immunized with COH04S1 clinical isolate developed comparable immune responses to young mice following prime-boost immunization.

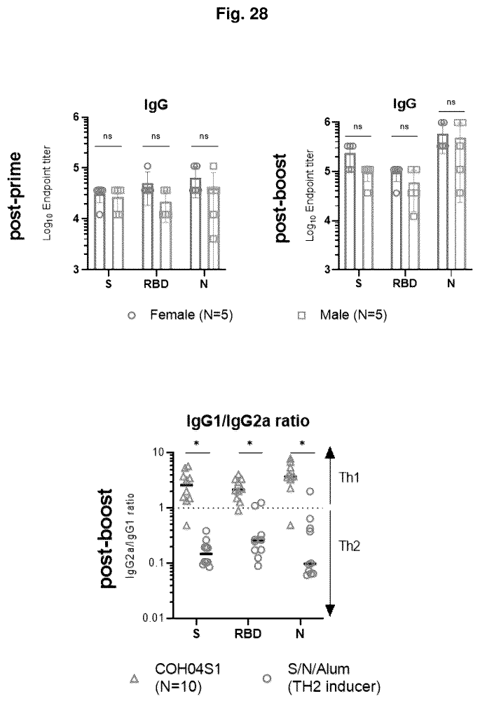

FIG. 28 shows the immunogenicity of COH04S1 clinical isolate. COH04S1 shows comparable immunogenicity between sexes in Balb/C mice and demonstrates Th1 immunity compared to S/N/Alum to all antigens.

### Example 8: In Vivo Immunogenicity of COH04S1 in Hamsters

COH04S1 immunogenicity and protection study was carried out using 6-8 weeks old golden Syrian hamsters (Mesocricetus auratus). The aim of the study was to test immunogenicity and protective efficacy of SARS-CoV-2 vaccine candidates based on the City of Hope (COH) synthetic MVA platform in golden Syrian hamsters.

The Syrian Golden Hamster was chosen as a small animal model due to the greater resemblance of compared model COVID-19 disease symptoms in this to human disease and in comparison to other small animal models, allowing for an assessment of the impact of the various vaccines including COH04S1 on preventatives and reduction in disease severity.

A total of 90 golden Syrian hamsters, evaluated in 15 groups described in Table 3 were used to evaluate synthetic SARS-CoV-2 sMVA vaccine candidates via the intramuscular and intranasal routes. In addition to COH04S1, sMVA constructs expressing wild-type or 2P S (Spike) and N (Nucleocapsid) or S alone were tested. This analysis included parental sMVA-N/S vector C35 co-expressing wild-type forms of S and N antigens (FIG. 5), clinical isolate COH04S1 (C35/F4/B1) derived by double plaque purification process from C35 (FIGS. 17 and 57), another double plaque-purified isolate (C35/F4/D5) derived from C35; a double plaque-purified isolate (C46/C3/F10) derived from sMVA-S/N vector C46 (FIG. 5), an sMVA vector co-expressing N together with a prefusion stabilized form of the S antigen with 2P alteration (C79), and sMVA-S vector C15 expressing only wild-type S. Control groups were sMVA empty vector and mock-immunized animals. Animals were immunized either intramuscularly (IM) or intranasally (IN) with 1×10⁸ pfu of sMVA recombinants.

TABLE 3
Study Groups/Experimental Design

| | | Treatment | | | Challenge | Weight and | Blood | |
|---|---|---|---|---|---|---|---|---|
| Gr | N | Material(Dose) | Route | Days | (IN) | Clin. Obs. | Collection | Termination |
| 1 | 6 (3F/3M) | sMVA (1e8) | IM | 0, 28 | Day 42 | Daily weights | Up to 5 | Day 52 |
| 2 | 6 (3F/3M) | C79 (S2P/N) (1e8) | IM | | SARS- | and BID | | Nasal wash |
| 3 | 6 (3F/3M) | COH04S1 (1e8) | IM | | CoV-2 | observation | | BAL |
| 4 | 6 (3F/3M) | C35/F4/D5 (S/N) (1e8) | IM | | 50 | during | | Collect lungs, |
| 5 | 6 (3F/3M) | C46/C3/F10 (S/N) (1e8) | IM | | μL/nostril | challenge | | trachea, nasal |
| 6 | 6 (3F/3M) | C15 (S) (1e8) | IM | | | period | | turbinates, |
| 7 | 6 (3F/3M) | C35 (S/N) (1e8) | IM | | | | | brain, kidney, |
| 8 | 6 (3F/3M) | sMVA (1e8) | IN | | | | | GI tract |
| 9 | 6 (3F/3M) | C79 (S2P/N) (1e8) | IN | | | | | |
| 10 | 6 (3F/3M) | COH04S1 (1e8) | IN | | | | | |
| 11 | 6 (3F/3M) | C35/F4/D5 (S/N) (1e8) | IN | | | | | |
| 12 | 6 (3F/3M) | C46/C3/F10 (S/N) (1e8) | IN | | | | | |
| 13 | 6 (3F/3M) | C15 (S) (1e8) | IN | | | | | |
| 14 | 6 (3F/3M) | C35 (S/N) (1e8) | IN | | | | | |
| 15 | 6 (3F/3M) | Mock | N/A | N/A | | | | |

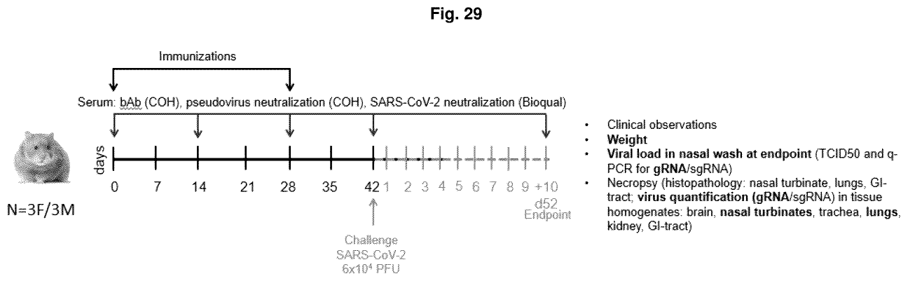

Vaccine constructs were administered to the animals via the indicated route at the specified dose on Day 0 followed by boost administration on day 28. Serum was evaluated for binding antibodies and SARS-CoV-2 authentic virus neutralization at the timepoints indicated (FIG. 29). Six animals per group (3 female and 3 male hamsters) were immunized in a prime-boost schedule with 1×10⁸ pfu of COH04S1 or 1×10⁸ pfu of sMVA empty control vector via the intramuscular or the intranasal routes.

Post-immunization analyses included detection of Spike- and Nucleocapsid-specific binding antibodies and quantification of neutralizing antibodies by both live SARS-CoV-2 virus and Spike-pseudovirus.

Two weeks post-boost, animals were challenged with 6×10⁴ pfu of SARS-CoV-2, Isolate USA-WA1/2020 (NR-52281, BEI Resources). At 10 days post challenge, the animals were euthanized, and the organs were collected for determination of virus titer, gross pathology and histopathological assessments. Weight loss over time and clinical observations were taken twice daily.

### Humoral Response

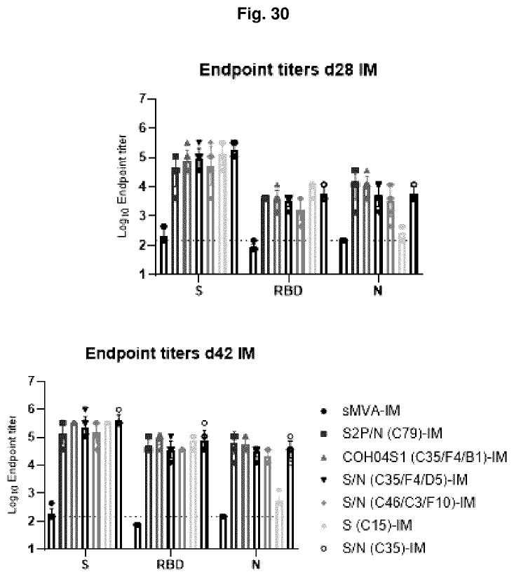

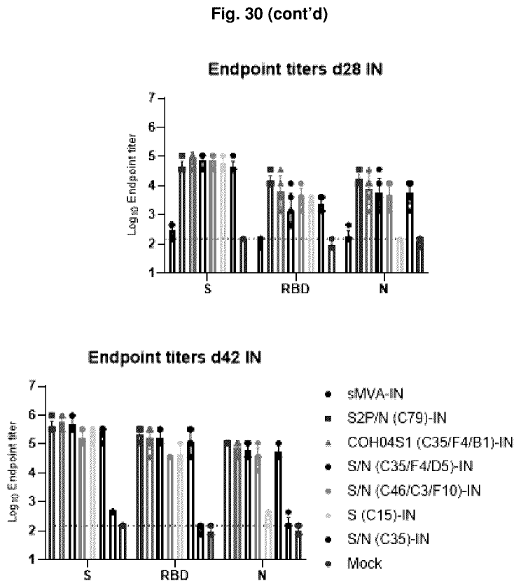

Total IgG binding antibodies to S, RBD and N were measured in hamster serum four weeks post-prime (day 28) and two weeks post-boost (day 42). Binding antibodies were not detected in control animals. In contrast, all sMVA-SARS-CoV-2 immunized animals developed binding antibodies to S, RBD, and N post-prime and titers were increased by a second dose (FIG. 30). Comparable titers were measured in IM- and IN-immunized hamsters.

Sera collected on days 28 and 42 were evaluated for the presence of neutralizing antibodies (NAb) using a PRNT SARS-CoV-2 assay.

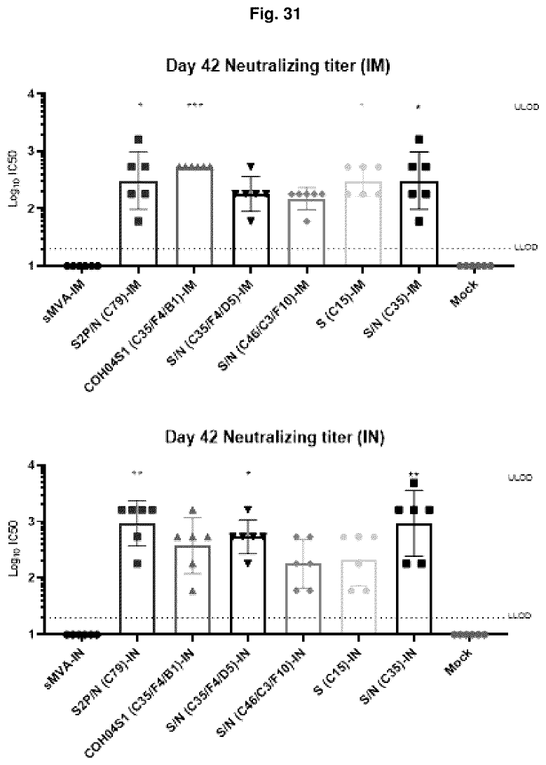

As shown in Table 4 and FIG. 31, control animals did not develop NAb (IC50 <20). Low titer NAb were detected in few animals post-prime and appeared to be higher after IN immunization although results are not available for all the animals. Post-boost NAb titers increased and ranged between 60 and >4860 (assay upper limit of detection).

TABLE 4
PRNT assay results

| | | | | Day 28 | Day 41 |
|---|---|---|---|---|---|
| Animal # | Group | Treatment | Route | Titer (IC₅₀) | Titer (IC₅₀) |
| 6310 | 1 | sMVA | IM | | <20 |
| 6311 | | | | | <20 |
| 6312 | | | | | <20 |
| 6313 | | | | | <20 |
| 6314 | | | | | <20 |
| 6315 | | | | | <20 |
| 6316 | 2 | S2P/N | IM | | 1620 |
| 6317 | | (C79) | | | 540 |
| 6318 | | 1e8 | | | 180 |
| 6319 | | | | | 60 |
| 6320 | | | | | 540 |
| 6321 | | | | | 180 |
| 6322 | 3 | COH04S1 | IM | | 540 |
| 6323 | | (C35/F4/B1) | | | 540 |
| 6324 | | 1e8 | | | 540 |
| 6325 | | | | | 540 |
| 6326 | | | | | 540 |
| 6327 | | | | | 540 |
| 6328 | 4 | S/N | IM | | 540 |
| 6329 | | (C35/F4/D5) | | | 180 |
| 6330 | | 1e8 | | | 180 |
| 6331 | | | | | 180 |
| 6332 | | | | | 60 |
| 6333 | | | | | 180 |
| 6334 | 5 | S/N | IM | | 180 |
| 6335 | | (C46/C3/F10) | | | 180 |
| 6336 | | 1e8 | | | 180 |
| 6337 | | | | | 180 |
| 6338 | | | | | 180 |
| 6339 | | | | | 60 |
| 6340 | 6 | S | IM | 20 | 180 |
| 6341 | | (C15) | | <20 | 180 |
| 6342 | | 1e8 | | 20 | 540 |
| 6343 | | | | 60 | 540 |
| 6344 | | | | <20 | 540 |
| 6345 | | | | <20 | 180 |
| 6346 | 7 | S/N | IM | <20 | 1620 |
| 6347 | | (C35) | | <20 | 180 |
| 6348 | | 1e8 | | <20 | 540 |
| 6349 | | | | <20 | 180 |
| 6350 | | | | <20 | 60 |
| 6351 | | | | <20 | 540 |
| 6352 | 8 | sMVA | IN | <20 | <20 |
| 6353 | | | | <20 | <20 |
| 6354 | | | | <20 | <20 |
| 6355 | | | | <20 | <20 |
| 6356 | | | | <20 | <20 |
| 6357 | | | | <20 | <20 |
| 6358 | 9 | S2P/N | IN | 60 | 180 |
| 6359 | | (C79) | | 180 | 540 |
| 6360 | | 1e8 | | 60 | 1620 |
| 6361 | | | | 180 | 1620 |
| 6362 | | | | 20 | 1620 |
| 6363 | | | | 180 | 1620 |
| 6364 | 10 | COH04S1 | IN | 180 | 1620 |
| 6365 | | (C35/F4/B1) | | 60 | 540 |
| 6366 | | 1e8 | | 20 | 540 |
| 6367 | | | | <20 | 180 |
| 6368 | | | | <20 | 540 |
| 6369 | | | | <20 | 60 |
| 6370 | 11 | S/N | IN | | 1620 |
| 6371 | | (C35/F4/D5) | | | 180 |
| 6372 | | 1e8 | | | 540 |
| 6373 | | | | | 540 |
| 6374 | | | | | 540 |
| 6375 | | | | | 540 |
| 6376 | 12 | S/N | IN | | 60 |
| 6377 | | (C46/C3/F10) | | | 540 |
| 6378 | | 1e8 | | | 180 |
| 6379 | | | | | 540 |
| 6380 | | | | | 60 |
| 6381 | | | | | 180 |
| 6382 | 13 | S | IN | | 180 |
| 6383 | | (C15) | | | 540 |
| 6384 | | 1e8 | | | 540 |
| 6385 | | | | | 60 |
| 6386 | | | | | 540 |
| 6387 | | | | | 60 |
| 6388 | 14 | S/N | IN | | 180 |
| 6389 | | (C35) | | | 180 |
| 6390 | | 1e8 | | | 1620 |
| 6391 | | | | | 1620 |
| 6392 | | | | | 1620 |
| 6393 | | | | | >4860 |
| 6394 | 15 | Mock | N/A | | <20 |
| 6395 | | | | | <20 |
| 6396 | | | | | <20 |
| 6397 | | | | | <20 |
| 6398 | | | | | <20 |
| 6399 | | | | | <20 |

### Body Weight Analysis

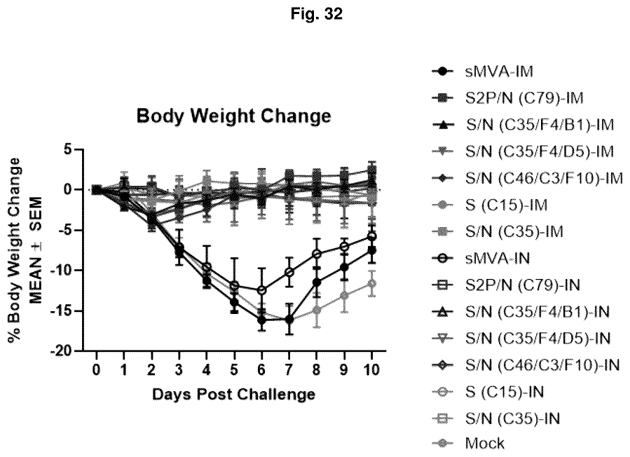

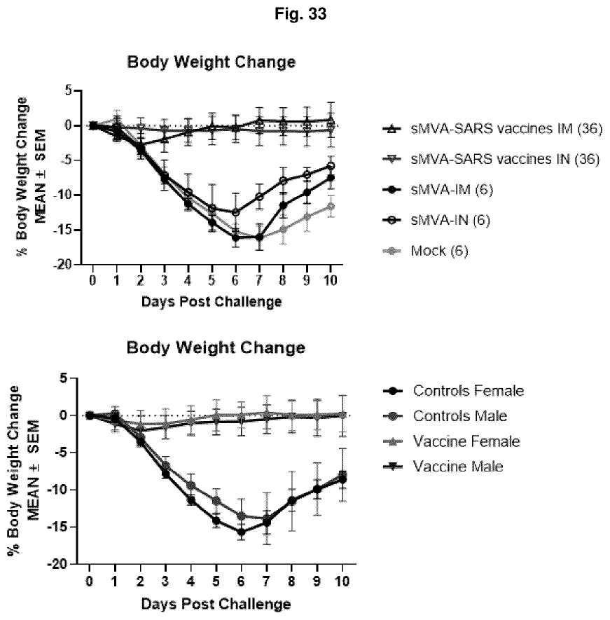

Hamsters were challenged two weeks post-boost with 6×10⁴ pfu of SARS-CoV-2, Isolate USA-WA1/2020, and the weight changes were measured daily for 10 days. Hamsters immunized IM with sMVA-S/N, N/S, S vaccines showed an initial minor weight loss comparable to control animals. Starting from day 3 post-challenge sMVA-S/N, N/S, S-immunized animals started recovering their weight while control animals kept losing weight. Control animals' weight dipped at day 7 to a mean value of −15% and started increasing thereafter. Between day 3 post-challenge and the final time-point 10-days post-challenge the difference in weight between sMVA-SARS-S/N, N/S, S and control animals was significant (FIGS. 32 and 33).

Similar results were obtained with sMVA-S/N, N/S, S-IN immunized animals. SMVA-S/N, N/S, S given intranasally prevented weight loss in challenged animals in comparison to mock-immunized and sMVA IN-immunized hamsters. The difference was significant from day 2 until the end of the study (FIGS. 32 and 33). No difference was observed amongst all tested recombinant vaccines, independently from the route of administration. Additionally, no difference in weight loss was observed between female and male hamsters (FIG. 33).

### Effects of COH04S1

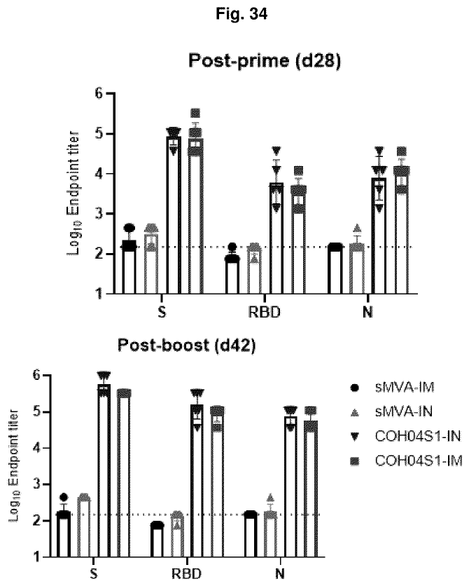

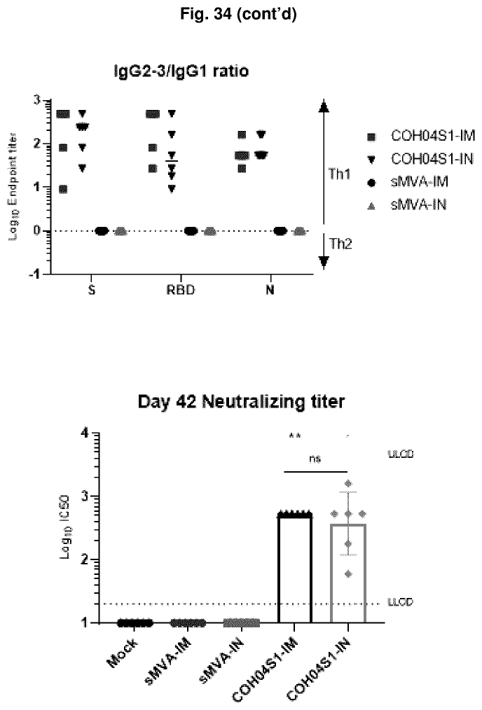

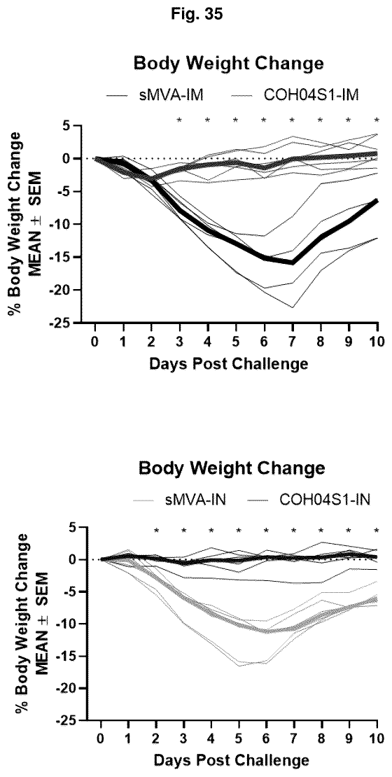

COH04S1 IM- and IN-immunized animals developed comparable binding antibody titers to S, RBD and N both post-prime and post-boost (FIG. 34). Additionally, analysis of the antibody isotype indicated a Th1-biased response with an IgG2-3/IgG1 ratio strongly shifted toward the Th1 Isotypes IgG2 and IgG3. Six out of six IM-immunized COH04S1 hamsters had high titer NAb with a geometric mean IC50 of 540. COH04S1 IN-immunized animals had titers between 180 and 1620 with a median titer of 540. COH04S1 administered IM or IN protected the animals from weight loss following a sub-lethal challenge with authentic SARS-CoV-2 virus (FIG. 35).

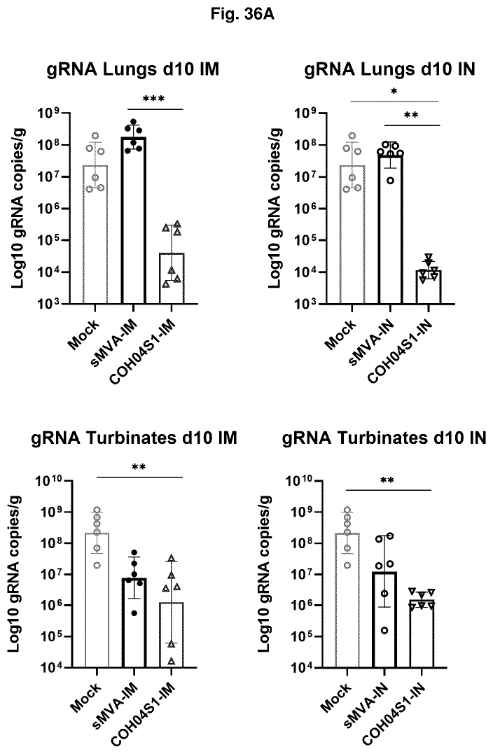

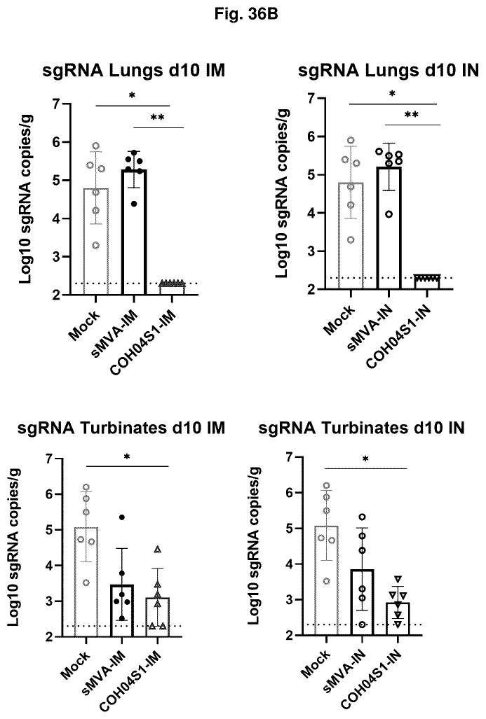

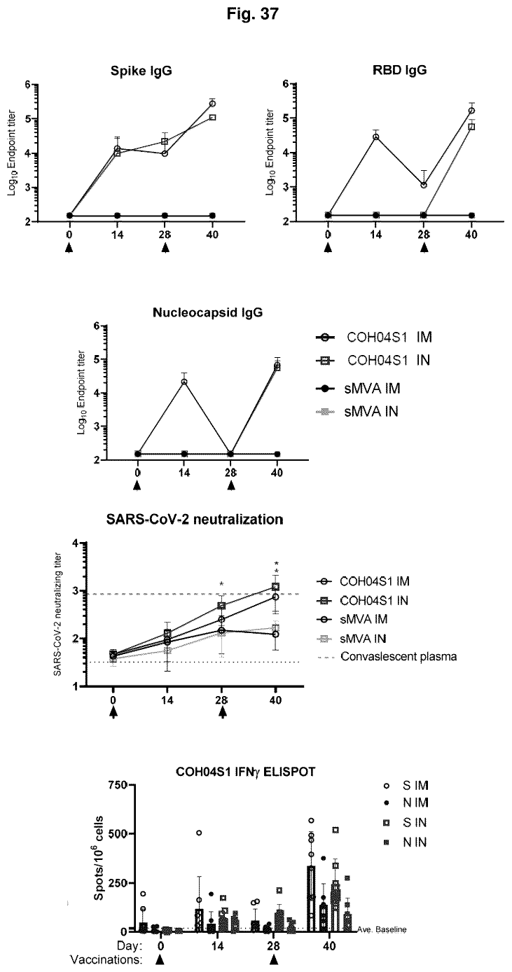

The lungs, turbinates and nasal wash collected at day 10 post-challenge were analyzed for the presence of SARS-CoV-2 genomes by genomic RNA qPCR (FIG. 36). At day 10 post-challenge, mock-immunized and sMVA-immunized hamsters still had high viral load in lungs, turbinates and in nasal washes. COH04S1 IM- and IN-immunized animals' samples showed significantly reduced gRNA amounts in lungs, turbinates and in nasal wash with the highest difference measured in lungs indicative of COH04S1-mediated protection.

### Example 9: In Vivo Immunogenicity and Protective Efficacy of COH04S1 in African Green Monkeys

African green monkeys (AGMs) support a high level of SARS-CoV-2 replication and develop pronounced respiratory disease that can be more substantial than in other NHP species including cynomolgus and rhesus macaques translating to greater comparability to symptoms of COVID-19 presentation in humans.

In this study, outbred AGMs of different sex and weight (Table 5) were vaccinated with COH04S1 intramuscularly (IM) with one or two doses and vaccine immunogenicity and protective efficacy were evaluated.

TABLE 5
AGM weight and sex distribution across groups

| Group | Study | ID# | Weight (kg) | Sex |
|---|---|---|---|---|
| 1 | 1 | D4697 | 4.76 | F |
| (Saline Control) | | D4649 | 3.02 | F |
| | | D4062 | 3.78 | F |
| | 2 | D4645 | 3.56 | F |
| | | D4756 | 3.45 | F |
| | | D3505 | 6.79 | M |
| 2 | 1 | D4187 | 4.03 | F |
| (Mock Vaccine, sMVA) | | D3783 | 3.31 | F |
| | | D3704 | 3.63 | F |
| | 2 | D4595 | 3.41 | F |
| | | D4079 | 3.73 | F |
| | | D3506 | 6.05 | M |
| 3 | 1 | C4403 | 4.02 | F |
| (COH04S1) | | D4877 | 2.91 | F |
| | | D4407 | 3.69 | F |
| | | D4389 | 3.68 | F |
| | | D4863 | 3.37 | F |
| | | D4732 | 7.23 | M |
| | 2 | D3308 | 3.79 | F |
| | | D4596 | 3.83 | F |
| | | D3808 | 2.95 | F |
| | | D4678 | 3.86 | F |
| | | D4640 | 3.36 | F |
| | | B2749 | 5.63 | M |

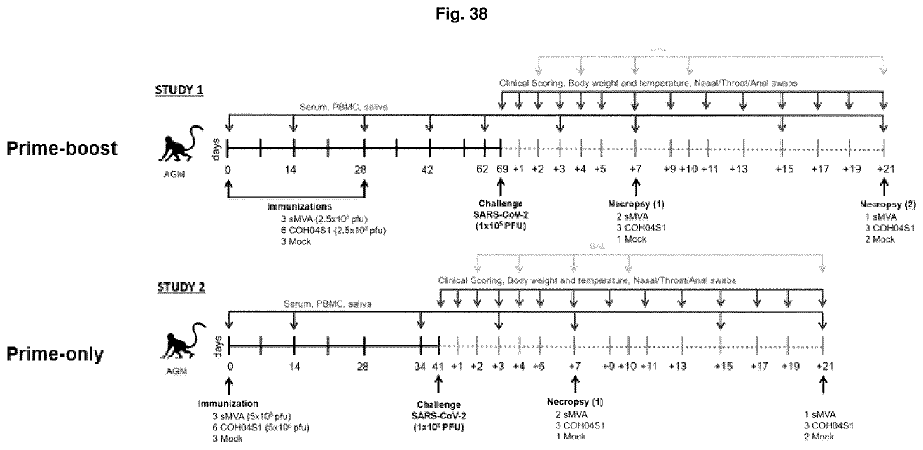

The AGMs received either one (study two) or two (study one) immunizations with 5×10⁸ pfu or 2.5×10⁸ pfu of sMVA recombinants, respectively. Three AGMs in each study received either mock saline immunization or empty sMVA vector as controls. Six AGMs in each study were immunized with COH04S1 in a prime (study 2) or prime-boost (Study 1) setting (FIG. 38). Blood and serum samples were collected at different timepoints for the analysis of cellular and humoral immunity. Six weeks post prime (study 2) or six weeks post-boost (study 1) the AGMs were challenged with 1×10⁵ pfu of SARS-CoV-2. The animals were observed, weighed and the temperature was taken daily for the first week and every other day for the following two weeks. On each sampling day, nasal, oral and anal swabs were collected. Broncho alveolar lavages (BAL) were collected on days 2, 4, 7, 10 and 21 post-challenge. At day 7 post-challenge 1 mock animal, 2 sMVA control animals and 3 COH04S1-immunized animals in both studies were sacrificed and organs were collected for virus quantification and histopathology. At day 21 post-challenge the remaining animals in both studies were sacrificed and organs collected for virological and immunological studies.

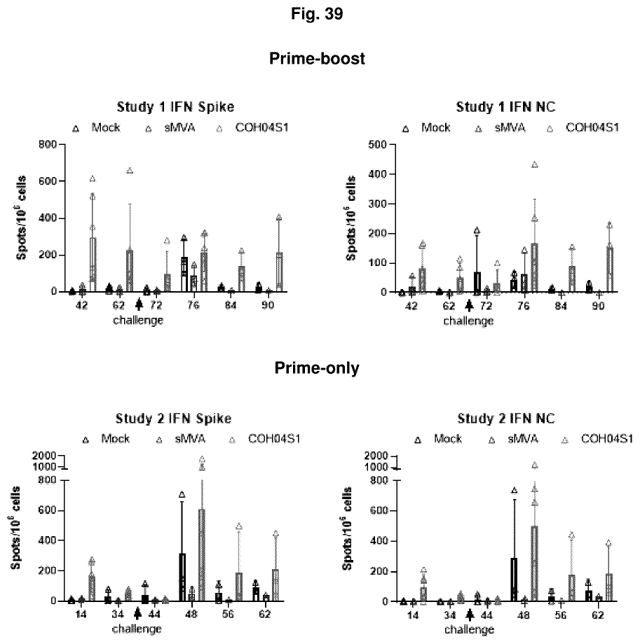

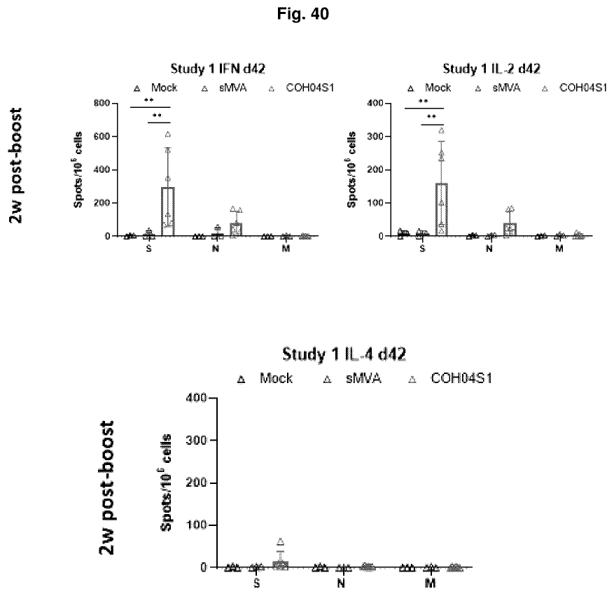

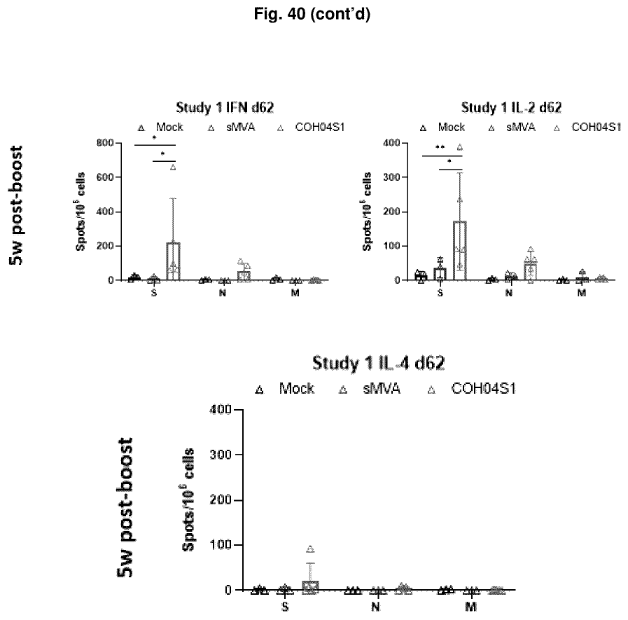

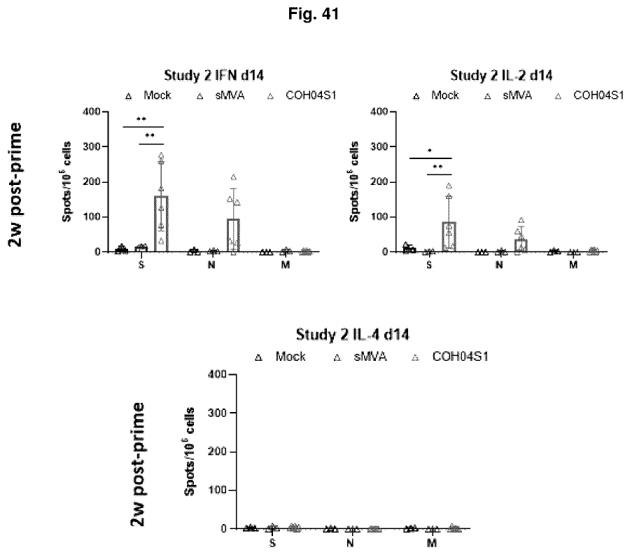

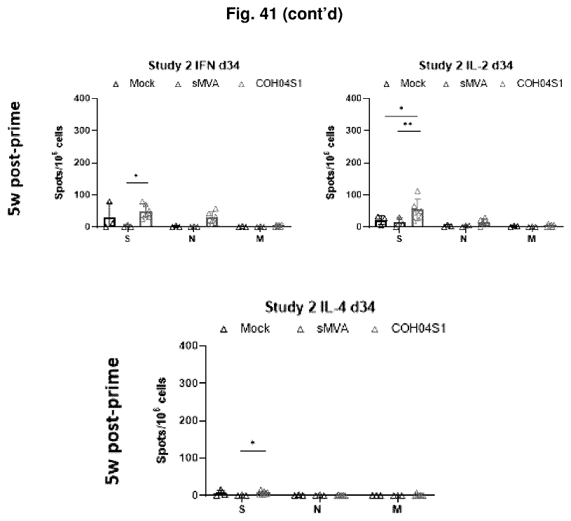

Starting from 2 weeks post-prime (study 2) and 2 weeks post-boost (study 1), T cell responses to Spike (S) and Nucleocapsid (N) antigens were evaluated in freshly isolated PBMCs by IFNγ/IL-2/IL-4 ELISPOT (FIGS. 39-41). Prime-only animals tended to have lower S- and N-specific IFNγ T cells levels than prime-boost animals. However, the recall response post-challenge was higher in prime only animals than in prime-boost animals. The COH04S1-immunized AGMs developed robust IFNγ T-cell responses to S and N that were absent in control animals and increased at day 7 post-challenge, indicative of an anamnestic response to the challenge virus (FIG. 39). IL-2 responses followed closely IFNγ responses although at lower levels. IL-4 T cell responses, indicative of a pathologic inflammatory response, were very low or absent in all COH04S1-immunized animals (FIGS. 40-41).

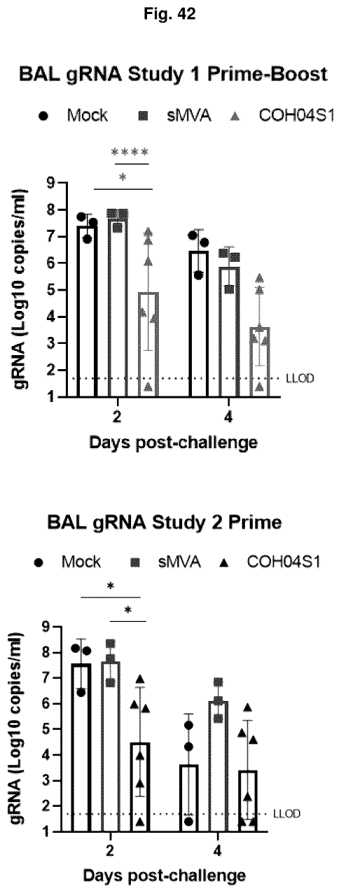

BAL samples were evaluated for the presence of SARS-CoV-2 challenge virus by genomic RNA (gRNA) quantification and plaque quantification (tissue culture infectious dose 50, TCID50). Differently from sub-genomic RNA (sgRNA) and TCID50 which only measure replicating virus, gRNA is a measure of both input challenge virus and replicating virus and especially at early time points post-challenge can be highly contaminated with input virus. At day 2 post-challenge, both prime and prime-boost COH04S1 animals showed significantly reduced gRNA copies in BAL samples than control animals (FIG. 42). One COH04S1-vaccinated animal in each study had undetectable virus in BAL. At day 4 post-challenge there was a trend to lower gRNA copies in vaccinated animals in comparison to controls but the difference was not significant.

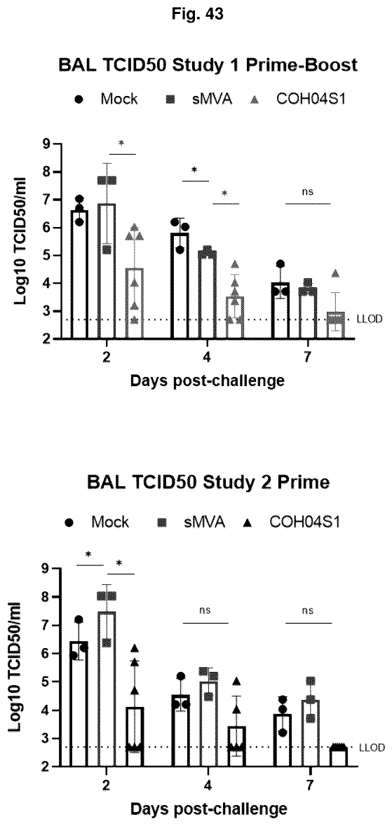

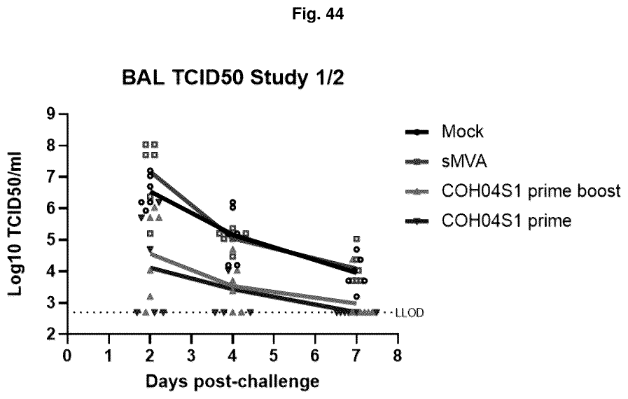

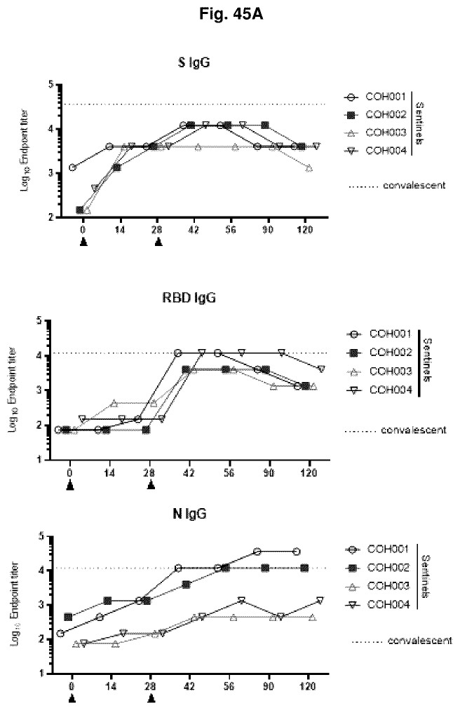

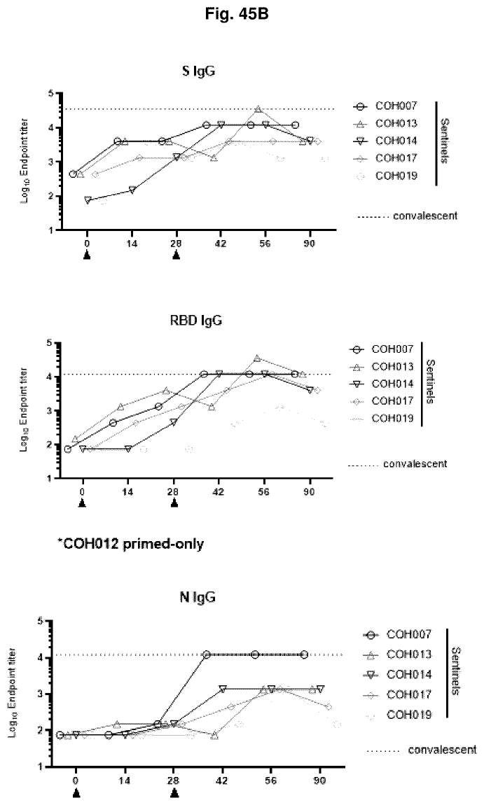

Viral load in BAL samples taken on days 2, 4, and 7 post-challenge was quantified by plaque assay (FIGS. 43-44). On day 2 post-challenge there was a significant reduction in vial load in samples from animals immunized with COH04S1 in comparison to controls. One animal on study one, and three animals on study 2 had undetectable virus in BAL samples on day 2 post-challenge. By day 7 post-challenge all COH04S1-immunized animals except for an AGM on study 1 had viral load below the lower limit of detection of the assay. In contrast, all control animals in both studies still had measurable virus in the lungs on day 7 post-challenge. These results demonstrate that COH04S1 administered either as one or two injections can rapidly protect AGM lower airways from SARS-CoV-2 virus infection.

### Example 10: Phase I Clinical Trial of COH04S1 for Prevention of COVID-19

COH04S1 was evaluated in healthy adults in a dose escalation clinical trial (NCT04639466) to identify adverse events and an optimal dose. Safety and tolerability of the COH04S1 vaccine were evaluated at three different dose levels (DLs): 1.0×10⁷ plaque-forming unit (PFU)/dose, 1.0×10⁸ PFU/dose, and 2.5×10⁸ PFU/dose. For each DL, 4-6 open label sentinels were included.

COH04S1 Phase I clinical trial was performed at 3 dose levels (DL1-3) with 4-6 open-label sentinels at each DL followed by 35 injected healthy research subjects randomized against placebo. DL1 corresponds to 1×10⁷ PFU/dose, same low dose as used in mice. DL2 corresponds to 1×10⁸ PFU/dose, and DL3 corresponds to 2.5×10⁸ PFU/dose. All doses are compatible with large scale production. Prime-boost immunizations were safely given to 16 out of 17 (one DL2 sentinel withdrew from the study after only receiving prime vaccination) sentinels, and COH04S1 was safe and well tolerated in DL1, DL2 and DL3 sentinels. All sentinels tested seroconverted to S and N antigens and developed Th1 T cell responses. All sentinels tested developed neutralizing antibodies.

### Binding Antibodies

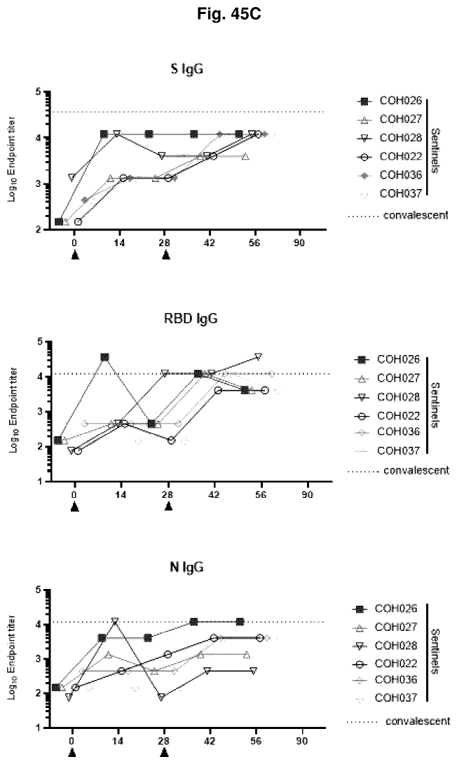

Four DL1 open label sentinels were evaluated for development of IgG binding antibodies to Spike (S), S receptor binding domain (RBD) and Nucleocapsid (N) up to day 120 using ELISA (FIG. 45A). In all DL1 sentinels at all time points, S-specific binding antibodies were measurable and tended to increase post-boost. RBD-specific binding antibodies were not present post-prime in 3/4 DL1 sentinels. Post-boost levels were comparable or slightly lower than median levels measured in a pool of 35 SARS-CoV-2 convalescent individuals that had mild-to-severe COVID-19 disease. N-specific binding antibody levels were variable amongst DL1 sentinels. Post-boost N-specific binding antibodies were detectable in all DL1 sentinels post-boost with levels as high as those measured in convalescent individuals.

Five DL2 sentinels were evaluated for development of IgG binding antibodies to S, RBD and N through day 90 (FIG. 45B). Similar to DL1 sentinels, in all five DL2 sentinels binding antibodies to S developed shortly after the first vaccination with COH04S1 and were boosted by a second dose. RBD-binding antibodies were measured in 4 out of 5 DL2 sentinels post-prime immunization and reached higher titers post-boost comparable to titers measured in convalescent serum. DL2 sentinels developed variable levels of N-specific binding antibodies with 5 out of 5 sentinels showing measurable levels of N-specific IgG by day 56.

S-, RBD-, and N-specific binding antibodies were evaluated in 6 DL3 sentinels up to day 56 (FIG. 45C). All DL3 sentinels readily developed S-specific binding antibodies post-prime. In 2 out of 6 DL3 sentinels S-specific IgG titers were not boosted by a second dose. In the remaining 4 DL3 sentinels S-IgGs increased after the boost at given day 28. RBD-specific binding antibodies were higher than baseline in 4 out of 6 DL3 sentinels, including one DL3 sentinel that post-prime reached higher titers than those measured in convalescent serum. Post-boost all DL3 sentinels had RBD-specific binding antibodies with titers approaching or comparable to titers measured in convalescent serum. N-specific binding antibodies were diverse amongst DL3 sentinels, although tended to reach higher titers post-prime in comparison to DL1 and DL2 sentinels. By day 42, 6 out of 6 DL3 sentinels had measurable N-specific binding antibodies.

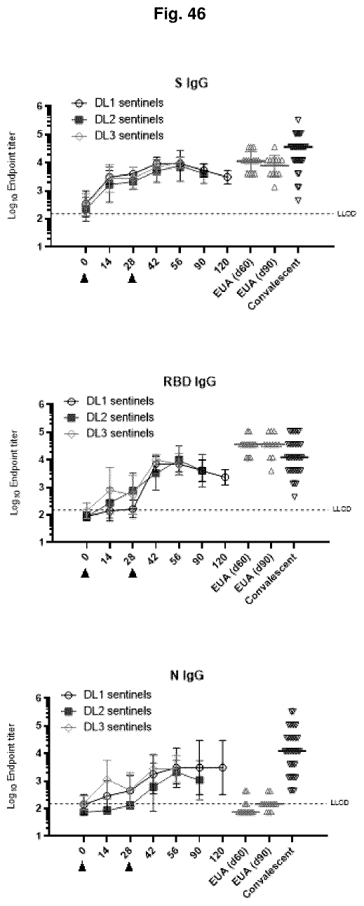

IgG titers to S, RBD and N in DL1/DL2/DL3 sentinels were compared to titers measured in a group of City of Hope employees who received two doses of EUA vaccine (Pfizer/BioNTech) at day 60 and 90 post prime immunization. Additionally, titers from COH04S1 vaccines were compared to titers measured in a pool of 35 SARS-CoV-2 convalescent individuals that had mild-to-severe COVID-19 disease (FIG. 46). Overall, S-, RBD- and N-specific antibodies induced after two doses of COH04S1 were comparable to those in EUA vaccine recipients and/or convalescent plasma from individuals recovered from mild-to-severe COVID-19 disease. Booster immunization increased titers especially for RBD binding antibodies.

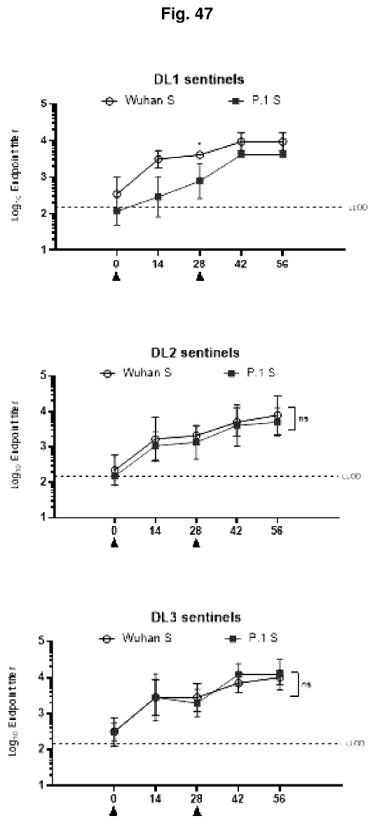

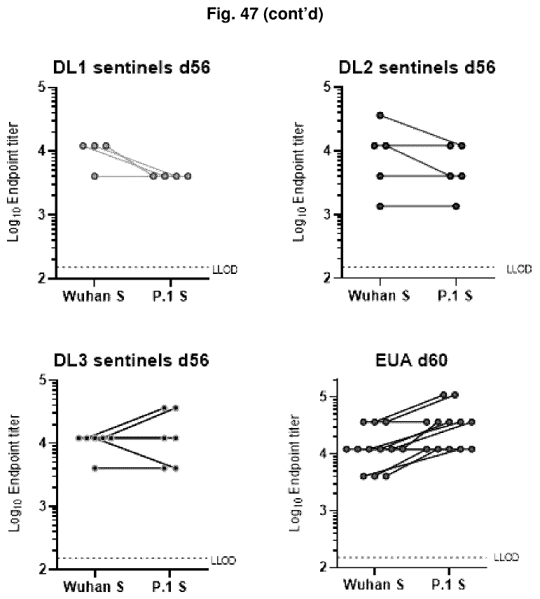

To address the most recent SARS-CoV-2 variant viruses, DL1/DL2/DL3 sentinel serum samples were evaluated for binding to P.1 Brazilian SARS-CoV-2 variant Spike and compared to binding to Spike from the original SARS-CoV-2 Wuhan strain (FIG. 47). DL1 sentinel serum samples bound less efficiently to P.1 Spike in comparison to Wuhan Spike resulting in lower titers. In contrast, at all timepoints, DL2 and DL3 sentinels had similar titers to P.1 Spike and Wuhan Spike with most DL3 sentinels having comparable titers or higher titers to P.1 Spike than Wuhan Spike at day 56.

### Neutralizing Antibodies

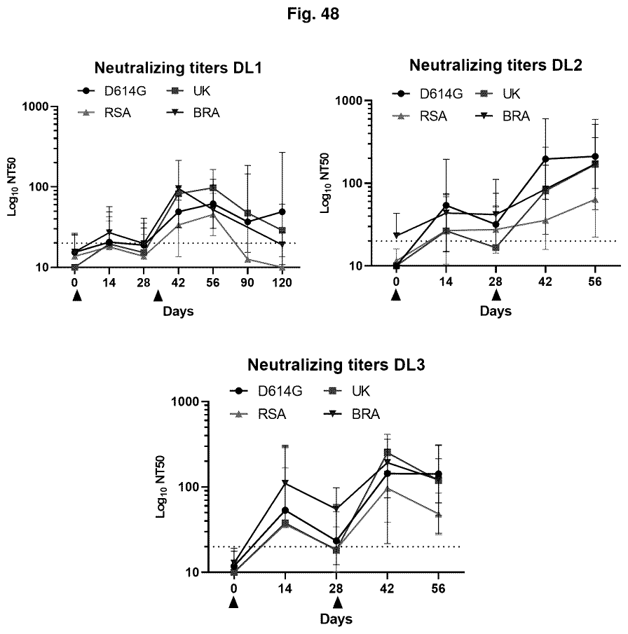

Neutralizing antibodies against the D614G variant of the ancestral Wuhan Spike amino acid sequence and against the widespread UK (B.1.1.7), the Republic of South African (RSA, B.1.351), and Brazilian (BRA, P.1) VOC were measured using an in vitro microneutralization assay and lentivirus-based pseudoviruses of each strain (FIG. 48). All Spike sequences included the D614G mutation and had a truncation of the last 19 amino acids at the C-terminus (KFDEDDSEPVLKGVKLHYT, SEQ ID NO: 65).

In concordance with the timing of development of RBD-specific binding antibodies, neutralizing antibodies against the three strains were low or not measurable post-prime in DL1 sentinels with the exception of one DL1 sentinel who immediately developed NT50 between 50 and 100 for the reference strain, UK and Brazilian VOC at day 14 post-prime. A significant increase in titers was observed in the other 3 DL1 sentinels post-boost reaching NT50 titers up to 150. All three strains were neutralized with variable potency and titers were stable through day 56. At days 90 and 120, all DL1 sentinels had measurable neutralizing antibodies for at least one viral strain (ancestral Wuhan strain or VOC).

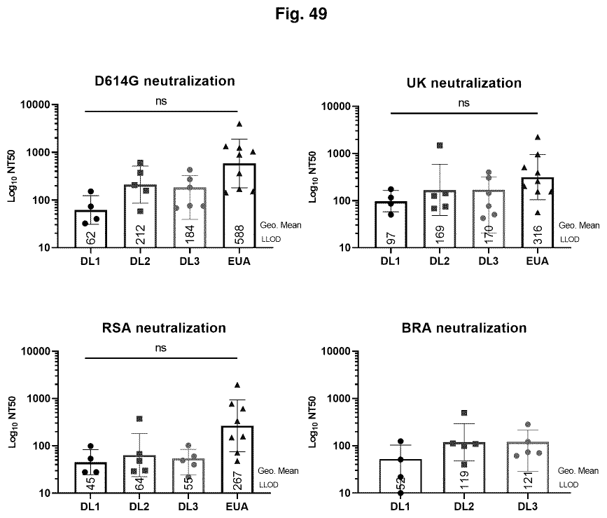

Of the 5 DL2 sentinels tested for neutralizing antibodies against the reference strain and the UK, RSA, and BRA VOC, 4 developed early neutralizing antibodies post-prime reaching peak NT50 titers up to 300. Overall, post-boost titers were more elevated than in DL1 sentinels with d56 NT50 geometric mean titers (GMT) titers of 212, 169, 64 and 119 against D614G (Wuhan), UK, RSA, and BRA VOC respectively (FIG. 49).

DL3 sentinels developed early high titer neutralizing antibodies in 2 out of 6 volunteers. The other 4 DL3 sentinels had low titer neutralizing antibodies post-prime which increased after a second dose of the vaccine. Overall, in DL3 sentinels titers of neutralizing antibodies to the D614G (Wuhan) strain and the UK, RSA, and BRA VOC was comparable to titers measured in DL2 sentinels and to titers measured using the same pseudoviruses in a cohort of EUA vaccine recipients (FIG. 49).

### T Cell Responses

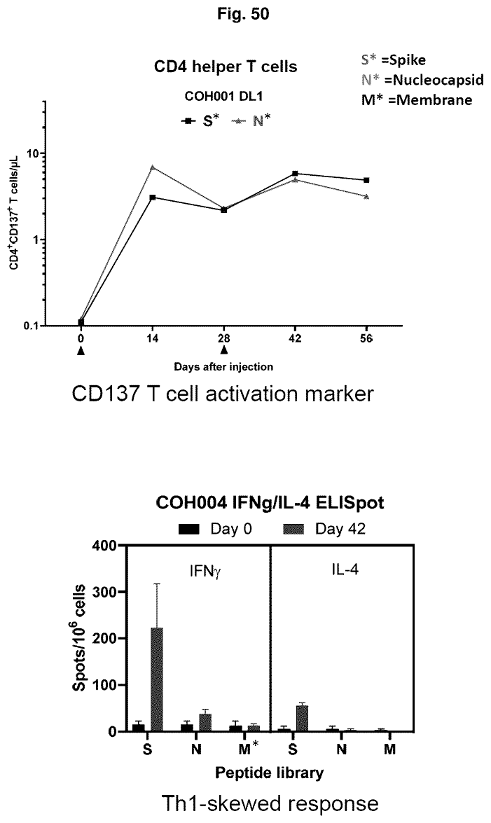

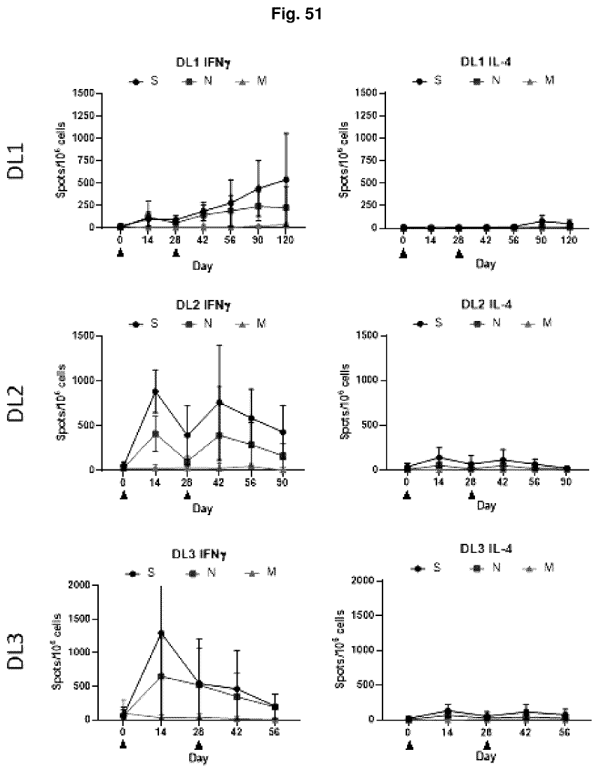

T cell responses were evaluated by IFNγ/IL-4 ELISPOT. Cryopreserved PBMCs were stimulated overnight in vitro with peptide pools covering the whole vaccine antigens S and N and additionally with SARS-CoV-2 viral membrane (M) antigen peptide pools. The Spike peptides were divided into four sub-pools with 71-86 peptides in each sub-pool and Elispot responses to each pool were added to give the total response to S antigen. All peptides covering N antigen were included in a single N antigen pool. Elispot responses in mock-stimulated samples (DMSO) were subtracted from each sample (FIGS. 50 and 51).

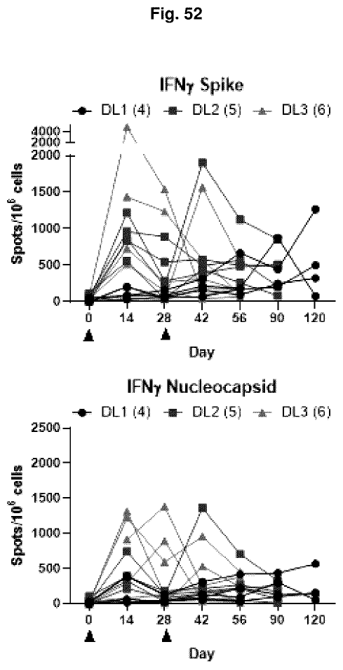

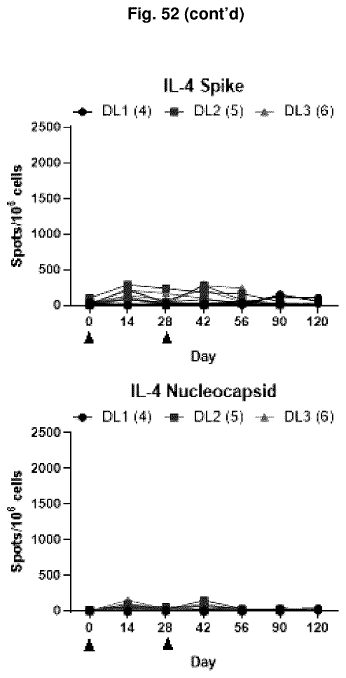

As shown in FIGS. 51 and 52, all 4 DL1 sentinels developed S- and N-specific IFN-γ T cell responses post-prime. T cells were boosted by a second immunization and were stable up to day 120 with a trend for higher S- and N-IFNγ responses. Levels of IL-4 secreting T cells were very low or absent after both S and N stimulation. M-specific response was absent in all DL1 volunteers. DL2 sentinels had higher IFN-γ T-cell response to both S and N than DL1 sentinels. Post-boost IFN-γ T cell responses were higher than post-prime for some subjects and lower for others. IL-4 response to S and N, suggestive of a Th2 phenotype of T helper cells, was low in all subjects. M-specific T cell responses were low or absent. DL3 sentinels IFN-γ T-cell response peaked post-prime and levels post-boost were lower than post-prime for most of the sentinels.

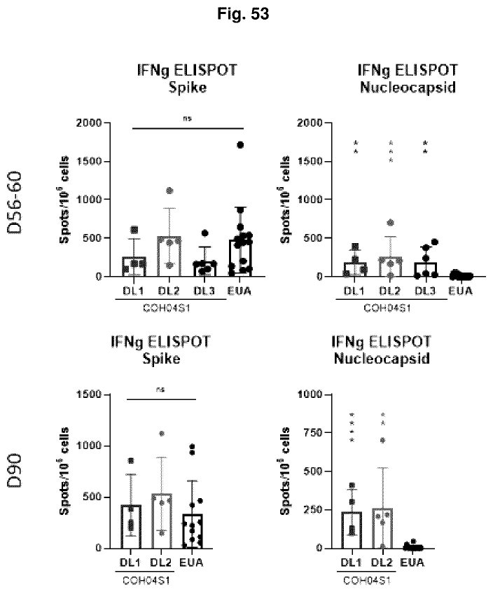

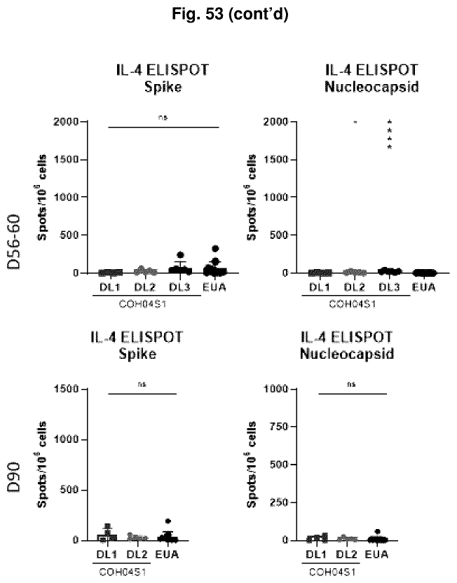

IFN-γ and IL-4 T cell responses in COH04S1 sentinels were compared to levels measured in a pool of Pfizer/BioNTech vaccine recipients at days 56-60 and 90 post prime-immunization (FIG. 53). At both day 56-60 and day 90 COH04S1 sentinels showed comparable levels of S-specific IFN-γ and IL-4 T cells to EUA vaccine recipients. N-specific IFN-γ T cell responses were significantly higher than in EUA vaccine recipients due to the inclusion of N antigen into COH04S1 but not in mRNA vaccines.

These results demonstrate that immunization with COH04S1 successfully induced strong Th1 T cell responses to S and N and desirably low Th2 responses. The T cell responses elicited by the vaccine compositions disclosed herein were comparable to other EUA and investigational vaccines.

### Example 11: Construction of Additional sMVA Vaccines Based on SARS-CoV-2 Variants

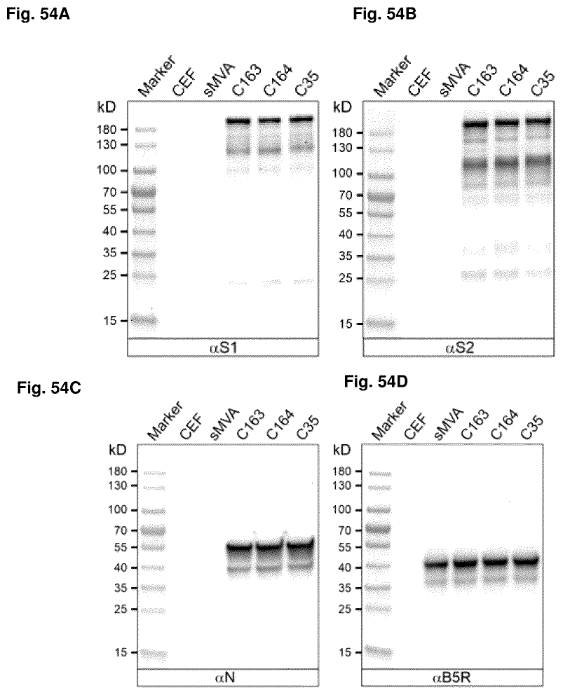

Using the synthetic vaccine platform, sMVA vectors co-expressing full-length S and N antigen sequences based on the SARS-CoV-2 variant lineage B.1.351 first identified in South Africa were generated. These sMVA constructs were derived from two independent virus reconstitutions and are herein referred to as C163 and C164. The C163 and C164 sMVA vectors were constructed as disclosed above similar to the C35 sMVA vaccine vector that formed the basis of the clinical product COH04S1, with the difference that the codon-optimized gene sequences based on the Wuhan reference strain that were inserted into the Del3 and Del2 site in C163 and C164 were further modified to encode for S and N antigens with several mutations specific for the B.1.351 lineage (see FIGS. 86 and 87 as well as 111 and 112 for specific sequences). The recombinant sMVA vectors included N501Y, E484K, K417N, L18F, D80A, D215G, Del242-244, R246I, D614G, and A701I mutations in the S antigen and a T205I mutation in the N antigen. Western Blot analysis confirmed the expression of the S1 and S2 domains of the S protein and the N protein by the C163/C164 variant vectors with similar expression levels compared to the original C35 vaccine construct (FIG. 54).

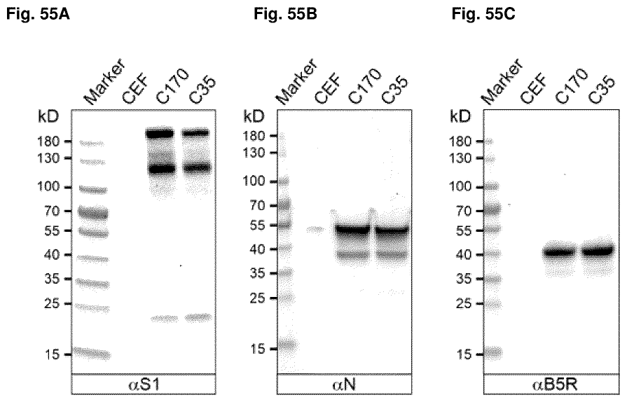

Using the synthetic vaccine platform, an sMVA vector co-expressing full-length S and N antigen sequences based on the SARS-CoV-2 variant lineage P.1 first identified in Brazil was generated. This sMVA construct is herein referred to as C170. The C170 sMVA vector was constructed as disclosed above similar to the C35 sMVA vaccine vector that formed the basis of the clinical product COH04S1, with the difference that the codon-optimized gene sequences based on the Wuhan reference strain that were inserted into the Del3 and Del2 site in C170 were further modified to encode for S and N antigens with several mutations specific for the P.1 lineage (see FIGS. 92 and 93 as well as 113 and 114 for specific sequences). The recombinant sMVA vector included N501Y, E484K, K417T, L18F, T20N, P26S, D138Y, R190S, H655Y, T1027I, and V1176F mutations in the S antigen and P80R, R203K, and G204R mutations in the N antigen. Western Blot confirmed the expression of the S1 and S2 domains of the S protein and the N protein by the C170 variant vector with similar expression levels compared to the original C35 vaccine construct (FIG. 55).

### REFERENCES

The references, patents and published patent applications listed below, and all references cited in the specification above are hereby incorporated by reference in their entirety, as if fully set forth herein.
1. Volz, A. & Sutter, G. Modified Vaccinia Virus Ankara: History, Value in Basic Research, and Current Perspectives for Vaccine Development. Advances in virus research 97, 187-243 (2017).
2. Gilbert, S. C. Clinical development of Modified Vaccinia virus Ankara vaccines. Vaccine 31, 4241-4246 (2013).
3. Sutter, G. & Moss, B. Nonreplicating vaccinia vector efficiently expresses recombinant genes. Proceedings of the National Academy of Sciences of the United States of America 89, 10847-10851 (1992).
4. Antoine, G., Scheiflinger, F., Dorner, F. & Falkner, F. G. The complete genomic sequence of the modified vaccinia Ankara strain: comparison with other orthopoxviruses. Virology 244, 365-396 (1998).
5. Meisinger-Henschel, C. et al. Genomic sequence of chorioallantois vaccinia virus Ankara, the ancestor of modified vaccinia virus Ankara. The Journal of general virology 88, 3249-3259 (2007).
6. Carroll, M. W. & Moss, B. Host range and cytopathogenicity of the highly attenuated MVA strain of vaccinia virus: propagation and generation of recombinant viruses in a nonhuman mammalian cell line. Virology 238, 198-211 (1997).
7. Cottingham, M. G. & Carroll, M. W. Recombinant MVA vaccines: dispelling the myths. Vaccine 31, 4247-4251 (2013).
8. Wussow, F. et al. A vaccine based on the rhesus cytomegalovirus UL128 complex induces broadly neutralizing antibodies in rhesus macaques. Journal of virology 87, 1322-1332 (2013).
9. Wussow, F. et al. Human cytomegalovirus vaccine based on the envelope gH/gL pentamer complex. PLoS pathogens 10, e1004524 (2014).
10. Chiuppesi, F. et al. Multiantigenic Modified Vaccinia Virus Ankara Vaccine Vectors To Elicit Potent Humoral and Cellular Immune Reponses against Human Cytomegalovirus in Mice. Journal of virology 92 (2018).
11. La Rosa, C. et al. MVA vaccine encoding CMV antigens safely induces durable expansion of CMV-specific T cells in healthy adults. Blood 129, 114-125 (2017).
12. Aldoss, I. et al. Poxvirus Vectored Cytomegalovirus Vaccine to Prevent Cytomegalovirus Viremia in Transplant Recipients: A Phase 2, Randomized Clinical Trial. Annals of internal medicine 172, 306-316 (2020).
13. Yuan, Y. et al. Complete regression of cutaneous metastases with systemic immune response in a patient with triple negative breast cancer receiving p53MVA vaccine with pembrolizumab. Oncoimmunology 6, e1363138 (2017).
14. Zhou, P. et al. A pneumonia outbreak associated with a new coronavirus of probable bat origin. Nature 579, 270-273 (2020).
15. Zhu, N. et al. A Novel Coronavirus from Patients with Pneumonia in China, 2019. The New England journal of medicine 382, 727-733 (2020).
16. Lurie, N., Saville, M., Hatchett, R. & Halton, J. Developing Covid-19 Vaccines at Pandemic Speed. The New England journal of medicine (2020).
17. Smith, T. R. F. et al. Immunogenicity of a DNA vaccine candidate for COVID-19. Nature communications 11, 2601 (2020).
18. Yu, J. et al. DNA vaccine protection against SARS-CoV-2 in rhesus macaques. Science (2020).
19. Zhu, F. C. et al. Safety, tolerability, and immunogenicity of a recombinant adenovirus type-5 vectored COVID-19 vaccine: a dose-escalation, open-label, non-randomised, first-in-human trial. Lancet (2020).
20. Ni, L. et al. Detection of SARS-CoV-2-Specific Humoral and Cellular Immunity in COVID-19 Convalescent Individuals. Immunity (2020).
21. Long, Q. X. et al. Antibody responses to SARS-CoV-2 in patients with COVID-19. Nature medicine (2020).
22. Premkumar, L. et al. The receptor binding domain of the viral spike protein is an immunodominant and highly specific target of antibodies in SARS-CoV-2 patients. Science immunology 5 (2020).
23. Hoffmann, M. et al. SARS-CoV-2 Cell Entry Depends on ACE2 and TMPRSS2 and Is Blocked by a Clinically Proven Protease Inhibitor. Cell 181, 271-280 e278 (2020).
24. Walls, A. C. et al. Structure, Function, and Antigenicity of the SARS-CoV-2 Spike Glycoprotein. Cell 181, 281-292 e286 (2020).
25. Wrapp, D. et al. Cryo-EM structure of the 2019-nCOV spike in the prefusion conformation. Science 367, 1260-1263 (2020).
26. Grifoni, A. et al. Targets of T Cell Responses to SARS-CoV-2 Coronavirus in Humans with COVID-19 Disease and Unexposed Individuals. Cell (2020).
27. Merchlinsky, M. Mutational analysis of the resolution sequence of vaccinia virus DNA: essential sequence consists of two separate AT-rich regions highly conserved among poxviruses. Journal of virology 64, 5029-5035 (1990).
28. Baroudy, B. M., Venkatesan, S. & Moss, B. Structure and replication of vaccinia virus telomeres. Cold Spring Harbor symposia on quantitative biology 47 Pt 2, 723-729 (1983).
29. DeLange, A. M., Reddy, M., Scraba, D., Upton, C. & McFadden, G. Replication and resolution of cloned poxvirus telomeres in vivo generates linear minichromosomes with intact viral hairpin termini. Journal of virology 59, 249-259 (1986).
30. DeLange, A. M. & McFadden, G. Efficient resolution of replicated poxvirus telomeres to native hairpin structures requires two inverted symmetrical copies of a core target DNA sequence. Journal of virology 61, 1957-1963 (1987).
31. Merchlinsky, M., Garon, C. F. & Moss, B. Molecular cloning and sequence of the concatemer junction from vaccinia virus replicative DNA. Viral nuclease cleavage sites in cruciform structures. Journal of molecular biology 199, 399-413 (1988).
32. Merchlinsky, M. & Moss, B. Resolution of linear minichromosomes with hairpin ends from circular plasmids containing vaccinia virus concatemer junctions. Cell 45, 879-884 (1986).
33. Wussow, F. et al. Exploiting 2A peptides to elicit potent neutralizing antibodies by a multi-subunit herpesvirus glycoprotein complex. Journal of virological methods 251, 30-37 (2018).
34. Scheiflinger, F., Dorner, F. & Falkner, F. G. Construction of chimeric vaccinia viruses by molecular cloning and packaging. Proceedings of the National Academy of Sciences of the United States of America 89, 9977-9981 (1992).
35. Taylor, J., Weinberg, R., Kawaoka, Y., Webster, R. G. & Paoletti, E. Protective immunity against avian influenza induced by a fowlpox virus recombinant. Vaccine 6, 504-508 (1988).
36. Mayr, A. & Malicki, K. [Attenuation of virulent fowl pox virus in tissue culture and characteristics of the attenuated virus]. Zentralblatt fur Veterinarmedizin. Reihe B. Journal of veterinary medicine. Series B 13, 1-13 (1966).
37. Tscharke, D. C. et al. Identification of poxvirus CD8+ T cell determinants to enable rational design and characterization of smallpox vaccines. The Journal of experimental medicine 201, 95-104 (2005).
38. Wyatt, L. S. et al. Elucidating and minimizing the loss by recombinant vaccinia virus of human immunodeficiency virus gene expression resulting from spontaneous mutations and positive selection. Journal of virology 83, 7176-7184 (2009).
39. Wyatt, L. S., Shors, S. T., Murphy, B. R. & Moss, B. Development of a replication-deficient recombinant vaccinia virus vaccine effective against parainfluenza virus 3 infection in an animal model. Vaccine 14, 1451-1458 (1996).
40. Wang, Z. et al. Modified H5 promoter improves stability of insert genes while maintaining immunogenicity during extended passage of genetically engineered MVA vaccines. Vaccine 28, 1547-1557 (2010).
41. Jaume, M. et al. Anti-severe acute respiratory syndrome coronavirus spike antibodies trigger infection of human immune cells via a pH- and cysteine protease-independent FcgammaR pathway. J. Virol. 85, 10582-10597 (2011).
42. Noyce, R. S., Lederman, S. & Evans, D.H. Construction of an infectious horsepox virus vaccine from chemically synthesized DNA fragments. PloS one 13, e0188453 (2018).
43. Wyatt, L. S., Earl, P. L. & Moss, B. Generation of Recombinant Vaccinia Viruses. Current protocols in protein science 89, 5 13 11-15 13 18 (2017).
44. Iwasaki, A. & Yang, Y. The potential danger of suboptimal antibody responses in COVID-19. Nature reviews. Immunology 20, 339-341 (2020).
45. Graham, B. S. Rapid COVID-19 vaccine development. Science 368, 945-946 (2020).
46. Crawford, K. H. D. et al. Protocol and Reagents for Pseudotyping Lentiviral Particles with SARS-CoV-2 Spike Protein for Neutralization Assays. Viruses 12 (2020).
47. Wang, Z. et al. Recombinant modified vaccinia virus Ankara expressing a soluble form of glycoprotein B causes durable immunity and neutralizing antibodies against multiple strains of human cytomegalovirus. J. Virol. 78, 3965-3976 (2004).
48. Tischer, B. K., von Einem, J., Kaufer, B. & Osterrieder, N. Two-step red-mediated recombination for versatile high-efficiency markerless DNA manipulation in Escherichia coli. Biotechniques 40, 191-197 (2006).
49. Tischer, B. K., Smith, G. A. & Osterrieder, N. En passant mutagenesis: a two step markerless red recombination system. Methods Mol. Biol. 634, 421-430 (2010).
50. Birnboim, H. C. & Doly, J. A rapid alkaline extraction procedure for screening recombinant plasmid DNA. Nucleic acids research 7, 1513-1523 (1979).
51. Earl, P. L., Moss, B., Wyatt, L. S. & Carroll, M. W. Generation of Recombinant Vaccinia Viruses. Current Protocols in Molecular Biology 43, 16.17.11-16.17.19 (1998).
52. Millet, J. K. et al. Production of Pseudotyped Particles to Study Highly Pathogenic Coronaviruses in a Biosafety Level 2 Setting. J Vis Exp (2019).
53. Rogers, T. F. et al. Isolation of potent SARS-CoV-2 neutralizing antibodies and protection from disease in a small animal model. Science 369, 956-963 (2020).

What is claimed is:

1. A vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-CoV-2 Nucleocapsid (N) protein, wherein the first DNA sequence is inserted in a first insertion site of the full-length sMVA genome; and
(iii) a second DNA sequence encoding a SARS-CoV-2 Spike (S) protein, wherein the second DNA sequence is inserted in a second insertion site of the full-length sMVA genome,
wherein the first DNA sequence encodes an amino acid sequence of SEQ ID NO: 10.

2. The vaccine composition of claim 1, wherein the full-length sMVA genome comprises a nucleotide sequence identical or substantially identical to MVA strain Antoine (Accession No. U94848).

3. The vaccine composition of claim 1, wherein the full length sMVA genome comprises a nucleotide sequence identical or substantially identical to MVA strain Acambis (Accession No. AY603355).

4. The vaccine composition of claim 1, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

5. The vaccine composition of claim 4, wherein the first insertion site is Del2.

6. The vaccine composition of claim 1, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

7. The vaccine composition of claim 6, wherein the second insertion site is Del3.

8. The vaccine composition of claim 1, wherein the rsMVA is reconstituted from homologous recombination of three DNA fragments, F1, F2, and F3, wherein:
F1 comprises a first partial sequence of the full-length sMVA genome and the first DNA sequence encoding the SARS-CoV-2 N protein;
F2 comprises a second partial sequence of the full-length sMVA genome; and
F3 comprises a third partial sequence of the full-length sMVA genome and the second DNA sequence encoding SARS-CoV-2 S protein;
and wherein an MVA terminal hairpin loop (HL) sequence flanked by MVA concatemeric resolution (CR) sequences (CR/HL/CR) is added to both ends of each of F1, F2, and F3.

9. The vaccine composition of claim 1, wherein the first DNA sequence is SEQ ID NO: 9.

10. A method of inducing an immune response to a SARS-CoV-2 virus in a human subject in need thereof comprising administering to the subject a vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-CoV-2 Nucleocapsid (N) protein, wherein the first DNA sequence is inserted in a first insertion site of the full-length sMVA genome; and
(iii) a second DNA sequence encoding a SARS-CoV-2 Spike (S) protein, wherein the second DNA sequence is inserted in a second insertion site of the full-length sMVA genome,
wherein the first DNA sequence encodes an amino acid sequence of SEQ ID NO: 10.

11. The method of claim 10, wherein the full-length sMVA genome comprises a nucleotide sequence identical or substantially identical to MVA strain Antoine (Accession No. U94848).

12. The method of claim 10, wherein the full length sMVA genome comprises a nucleotide sequence identical or substantially identical to MVA strain Acambis (Accession No. AY603355).

13. The method of claim 10, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

14. The method of claim 13, wherein the first insertion site is Del2.

15. The method of claim 10, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

16. The method of claim 15, wherein the second insertion site is Del3.

17. The method of claim 10, wherein the rsMVA is reconstituted from homologous recombination of three DNA fragments, F1, F2, and F3, wherein:
F1 comprises a first partial sequence of the full-length sMVA genome and the first DNA sequence encoding the SARS-CoV-2 N protein;
F2 comprises a second partial sequence of the full-length sMVA genome; and
F3 comprises a third partial sequence of the full-length sMVA genome and the second DNA sequence encoding SARS-CoV-2 S protein;
and wherein an MVA terminal hairpin loop (HL) sequence flanked by MVA concatemeric resolution (CR) sequences (CR/HL/CR) is added to both ends of each of F1, F2, and F3.

18. The method of claim 10, wherein the first DNA sequence is SEQ ID NO:9.

19. A method of preventing a SARS-CoV-2 virus infection in a human subject in need thereof comprising administering to the subject a vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-CoV-2 Nucleocapsid (N) protein, wherein the first DNA sequence is inserted in a first insertion site of the full-length sMVA genome; and
(iii) a second DNA sequence encoding a SARS-CoV-2 Spike (S) protein, wherein the second DNA sequence is inserted in a second insertion site of the full-length sMVA genome;
wherein the first DNA sequence encodes an amino acid sequence of SEQ ID NO: 10.

20. The method of claim 19, wherein the full-length sMVA genome comprises a nucleotide sequence identical or substantially identical to MVA strain Antoine (Accession No. U94848).

21. The method of claim 19, wherein the full length sMVA genome comprises a nucleotide sequence identical or substantially identical to MVA strain Acambis (Accession No. AY603355).

22. The method of claim 19, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

23. The method of claim 22, wherein the first insertion site is Del2.

24. The method of claim 19, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

25. The method of claim 25, wherein the second insertion site is Del3.

26. The method of claim 19, wherein the rsMVA is reconstituted from homologous recombination of three DNA fragments, F1, F2, and F3, wherein:
F1 comprises a first partial sequence of the full-length sMVA genome and the first DNA sequence encoding the SARS-CoV-2 N protein;
F2 comprises a second partial sequence of the full-length sMVA genome; and
F3 comprises a third partial sequence of the full-length sMVA genome and the second DNA sequence encoding SARS-CoV-2 S protein;
and wherein an MVA terminal hairpin loop (HL) sequence flanked by MVA concatemeric resolution (CR) sequences (CR/HL/CR) is added to both ends of each of F1, F2, and F3.

27. The method of claim 19, wherein the first DNA sequence is SEQ ID NO:9.

28. A vaccine composition comprising a recombinant modified vaccinia Ankara (rMVA) virus, the rMVA comprising:
(i) a first DNA sequence encoding a SARS-CoV-2 Nucleocapsid (N) protein, wherein the first DNA sequence is inserted in a first insertion site of a full-length MVA genome; and
(ii) a second DNA sequence encoding a SARS-CoV-2 Spike (S) protein, wherein the second DNA sequence is inserted in a second insertion site of the full-length MVA genome,
wherein the first DNA sequence encodes an amino acid sequence of SEQ ID NO: 10.

29. The vaccine composition of claim 28, wherein the first DNA sequence is SEQ ID NO: 9.

30. The vaccine composition of claim 28, wherein the first insertion site is Del2.


SEQUENCE LISTING (90 sequences)

SEQ ID NO: 1 - DNA, 171907 bp, synthetic construct
  misc_feature 1..171907: note = sMVA-N/S
  source 1..171907: mol_type = other DNA
ttttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatatttttgtatgagaccatcagaaagaggtttaatatttttgtgataccctgaaaggaaataggaatagtgtcataatcgtatcacactattgagacagaaaaagaagaagtcgcgagaggtaactttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttccatcagcggattctgagatggatttaatctgaggacatttggtgaatccaaagttcattctcagacctccaccgatgatggagtaataagtggtaggaggatctacatcctcgactgattccacctcgggatctggatctgactcggactctgtaatttccgttacggattggcaaatcttatcatcggtcggtgtttggtcttgctttgtgactttgataataacatcgattcccatatgatgtttgttttcttcttccgtacacgatgaggatgattgctgaagactggcaggcacatgcatgccagtacgatatattgtttcatgattgctattgattgagtactgttctttatgattctacttccttaccgtgcaataaattagaatatattttctacttttacgagaaattaattattgtatttatgggtgaaaaacttactataaaaagcgggtgggtttggaattagtgatcagtttatgtatatcgcaactaccgggcatatggctacattacccacatgataagagattgtatcagtttcgtagtcttgagtattggtattactatatagtatatagatgtcgacgctagagttactgtctccgaatgcggcatgatagtatcattctttgctttcgttaactgtttggaggaagaatctttgttattgcatttaatctcgaaattcagagtgcacacctttctcctgtaaagaaacctgaagtcgctaccttattaagaagacgggatcgcagtctttatgattcatagtaatagttagttccgacgttgagatggattcgctgagaccggtagtggtcgtccgagtacacgatgtgtcgttaactggatacaggttaatttccacatcgatatagttaaaggtatttctgggtacgggttcgcatttatctgcggaagagacggtgtgagaatatgttccgagaccacacggagaacagatgacgtctccggatactccgtatcctattccacattttgtttgggaaacacatgccttgcatccatgatcgggagagcattcacagattctattgtgagtcgtgttacacgatcgcgtcgacattgttgacagaaacgtgaccttcattcttaccgtcgtccataaatacgttaggtatgtaccacatactgtcgcgaacgatgcgtccatctcataatgatttactttttcataattaaagatgtgaaagaaaaccgaacaatatatttttttagtaatgtttatgcgagacatataaaataaactccgtgtttatgatgccggtaaatgtttttatcatcttggacggaatcgattttgtaatatgccatggaaacaggacattatcactccatgataaattatttaatggagtcgatcctctcattgttctttgcgtatctcaatctgtggcgtttgcttcgtttaaataatatatcaaacatggagacgcctgatatgtaggcattcttcattctattaatgtctgctctatagcgctttagttccttatgacgaccggcgatatcatacttactttagaaggaaaatcatcatctaggattaaggcgtatctgatacaggcgaataatggttcaggatatagatagcgtatatctctattaaatgcgtcaatcatagtctctagagtgggatggtaactcagtaataaatcaactagcttctctttggtaactgcttttctggatggccgtattgattatcgagcgtgacactcgctccatattccaataaccgctttgcaaattgtatattattgacatcgaccgcgtaatatagtagagttatcgatcatatctatatcatccatgtacttgcttagtatatcaaatacatcttcataacagtgatacccgcaattattaaatctcgataatatcagaccgtacatacatagacggccattgttagatatgtgatttacagccgcgtgtccatattttccacgataaaccttacgacgtttacatcgacgagattattattaacaaagttgttgtccgtcgtcttatccaacatgcattgaatgataggtatacttaccatatcgccgtaatgtaagtagtttatcagtatggcttgtacgatggattcatcctgttgtctaaatctctttagaatgttatcgatgatgtagtggttatattctctggaatcgtacgaagtaatactacgcattacgtcgacaagagtatgacgtctctcaataagaagattaacgatttccatgtctacattatatggggttactctaaatcgcttgtttagataatacgcctctaatatagggctgacgtcgtatactctacacgtgtccacatcctttattaataatctctatatctatggttgagcaagaccagtagtattggatggaaacattgttatcgatcaaacatttaattacatccttggatagagattctctatgagacgatatatagtaatgaagagagttcttacacatatcactgttgtacatacaggtacgaaatacgtaaccggtgctgtaacattctgatttaagaagccatagcaatacttctggtctcggattaggcgtcgttacgtatatatccaccaatccgagaccattgattgcataattcgtattcttggacggacgtatccgtttatccacaattaggtattttagcagacgtaagtcgaaatcatttatattcgacttgagttcgttagaggaattcgaatagctggatatcagtagatgcacaatctgagattttacgtatctatgcttactgtatgctcctagcggagttaatccttcgttgtttctacaaagtctctcgactccgcgagagagtaacagtcgaacaatcttaatgtctgtatcgcatttattggagacgtaacaatgtagcgcattgtttcctcgtctatctatatgttttgataagttgtgacacgtttcaatttctagttttatttttttgtacgtcacatcttcatccagtagacgacatagaatacatgtgcaatccatagctattctggtgctaattattcctcataagatgataaaaagtgtagtgagagagcatgaaggagatttagtatttagcagtgcggatatgatccaagagggtgagatagtcgttctcgttcagaatctttcgcagcataagtagtatgtcgatatacttatcgttgaagactcttccagagacgatagctgattgagtacaaagtccaatgattgcacgaagttcttcggcggttttcatggagtcatttctgatgaaacatttaatgatctaaatttcagtttatgtttgtaccccgtattcatacttaacaaattggtattacataccattaataatgcaagcataaaaaatcgttagtagatgtttctaaatataggttccgtaagcaaagaatataagaatgaagcggtaatgataaaatcaatcgttatctaaaatgatcatactcatttattttattctattatattaacacatacatttttaacagcaacacattcaatattgtattgttatttttatattatttacacaattaacaatatattattagtttatattactgaattaataatataaaattcccaatcttgtcataaacacacactgagaaacagcataaacacaaaatccatcaaaaatgttgataaattatctgatgttgttgttcgctgctatgataatcagatcattcgccgatagtggtaacgctatcgaaacgacatcgccagaaattacaaacgctacaacagatattccagctatcagattatgcggtccagagggagatggatattgtttacacggtgactgtatccacgctagagatatcgacggtatgtattgtagatgctctcatggttatacaggcattagatgtcagcatgtagtattagtagactatcaacgttcagaaaaaccaaacactacaacgtcatatatcccatctcccggtattatgcttgtattagtaggcattattattattacgtgttgtctattatctgtttataggttcactcgacgaactaaactacctatacaagatatggttgtgccataatttttataaattttttttatgagtatttttacaaaaatgtataaagtgtatgtcttatgtatatttataaaaatgctaaatatgcgatgtatctatgttatttgtatttatctaaacaatacctctacctctagatattatacaaaaattttttatttcggcatattaaagtaaaatctagttaccttgaaaatgaatacagtgggtggttccgtatcaccagtaagaacataatagtcgaatacagtatccgattgagattttgcatacaatactagtctagaaagaaatttgtaatcatcttctgtgacgggagtccatatatctgtatcatcgtcccatgctatattcctgttatcatcattagttaatgaaaataactctcgtgcttcagaaaagtcaaatattgtatccatacatacatctccaaaactatcgcttatacgtttatctttaacgatacctatacctagatggttatttactaacagacattttccagatctattgactataactcctatagtttccacatcaaccaagtaatgatcatctattgttatataacaataacataactcttttccgtttttatcagtatgtatatctatatcaacgtcgtcgttgtagtgaatagtagtcattgatctattatatgaaacggatatgtctagaacggcaattgttttacgtccagttaacactttcgttgatttaaagtctagagtctttgcaaacataatatccttatccgactttatatttcctgtagggtggtataattttattttgcctccacatatcggtgtttccaaatatattactagacaatattccatatagttattagttaagggtacccaattagaacacgtacgcttattatcatcatttggatcgtatttcataaaagttattgtactatcgatgtcaacacattctacattttttaatcgtctatatagtatttttctgatattttctataatatcagaattgtcttccatcggaagttgtatactatcggaatcagttacatgtttaaataattctctgatgtcattccttatacaatcaaattcattattaaacagtttaatagtctgtagacctttatcgtcgtaaatatccattgtcttattagttacgcttatttttatgtgtttttacgttgctttattatattttataagaatgattgtttgacgaatcacgagaactattaagacacattattaggtatatattataaaaaagtttttgattacgatgttataagaggaaagaggacacattaacatcatacatcaattaactacattcttataacatcgtaatcaaaagaattgcaattttgatgtataacaactgtcaatgggttatggaattgtatattacatattatacggtatgttggtaacgacaaataccgatcggtaattgtctgccggtgtaatagaattatatatatctatctattacaccggccttgtatacataataataagttgtggtagtatgatctccatatttataatttaggactttgtattcagtttttttggaatcataaaaaataaaaaaaagttttactaatttaaaattatttacatttttttcactgtttagtcgcggatatggaattcgatcctgccaaaatcaatacatcatctatagatcatgtaacaatattacaatacatagatgaaccaaatgatataagactaacagtatgcattatcacaaaaataaatccacatttggctaatcaatttcgggcttggaaaaaacgtatcgccggaagggactatatgactaacttatctagagatacaggaatacaacaatcaaaacttactgaaactgtcaaaaaaatagaaacatatatggtctatatatacactacaatttagttattaattggataaccgatgtgattatcaatcaatattaagaaggttggtaaattggtacatagctaataatacctatacacccaataatacaacaaccatttctgagttggatatcatcaaaatactggataaatacgaggacgtgtatagagtaagtaaagaaaaagaatgtgaaatttgctatgaagttgtttactcaaaacgatagatactttggtttattggattcgtgtaatcatatattttgcataacatgcatcaatatatggcatagaacacgaagagaaaccggtgcgtcggataattgtcctatatgtcgtacccgttttagaaacataacaatgagcaagttaactaataaataaaaagtttaatttgttgacgacgtatgtcgttattttttctcgtataaaagattaatttgattctaatataatctttagtattggataaatatcaattcaaattaattccattagattatatcataaataaaaatagtagcacgcactacttcagccaaatattcttttttgaaacgccatctatcgtagtgaggacacaagtgaacctataatgagcaaatttattagtatcggttacatgaaggactttacgtagagtggtgattccactatctgtggtacgaacggtttcatcttctttgatgccatcacccagatgttctataaacttggtatcctttgccaaccaatacatatagctaaactcaggcatatgttccacacatcctgaacaatgaaattctccagaagatgttacaatgtctagatttggacatttggtttcaaccgcgttaacatatgagtgaacacacccatacatgaaagcgatgagaaataggattttcatcttgccaaaatatcactagaaaaaatttatttatcaattttaaaggtataaaaaatacttattgttgctcgaatattttgtatttgatggtatacggaagattagaaatgtaggtattatcatcaactgattctatggttttatgtattctatcatgtttcactattgcgttggaaataatatcatatgcttccacatatattttattttgttttaactcataatactcacgtaattctggattattgacatatctatgaataattttagctccatgatcagtaaatattaatgagaacatagtattaccacctaccattatttttttcatctcattcaattcttaattgcaaagatctatataatcattatagcgttgacttatggactctggaatcttagacgatgtacagtcatctataatcatggcatatttaatacattgttttatagcatagtcgttatctacgatgttagatatttctctcaatgaatcaatcacacaatctaatgtaggtttatgacataatagcattttcagcagttcaatgtttttagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacatgtcacaaagaccctttttgtatgtataagtgtaaaaattataacattcatagttggatttacataggtgtccaatcgggatctctccatcatcgagataattgatggcatctcccttccttttttagtagatatttcatcgtgtaagaatcaatattaatatttctaaagtatccgtgtatagcctctttatttaccacagttccatattccactagagggatatcgccgaatgtcatatactcaattagtatatgttggaggacatccgagttcattgttttcaatatcaaaaagatggtttccttatcatttctccatagtggtacaatactacacattattccgtgcggctttccattttccaaaaacaatttgaccaaatctacatctttattgtatctataatcactatttagataatcagccataattactcgagtgcaacatgttagatcgtctatatatgaataagccgtgttatctattcctttcattaacaatttaacgatgtctatatctatatgagatgacttaatataatattgaagagctgtacaatagtttttatctataaaagacggcttgattccgtgattaattagacatttaacaacttccggacgcacatatgctctcgtatccgactctgaatacagatgagagatgatatacagatgcaatacggtaccgcaatttcgtagttgataatcatcatacgcgtatcagtactcgtcctcataaagaacactgcagccattttctatgaacaaatcaataatttcaggaacaggatcatctgtcattacataattttctataactgaacgatggttttcacatttaacactcaagtcaaatccatgttctaccaacacctttatcaagtcaacgtctacatttttggatttcatatagctgaatatattaaagtcatttatgttgctaaatccagtggcttctagtagagccatcgctatatcctttaactttaacatgtctactatttgtgtattcttctaatggggtagctgtctccaatttttgcgtaatggattagtgccactgtctagtagtagtttgacgacctcgacattattacaatgctcattaaaaaggtatgcgtgtaaagcattattcttgaattggttcctggtatcattaggatctctgtctctcaacatctgtttaagttcatcgagagccacctcctcattttccaaatagtcaaacattttgactgaatgagctactgtgaactctatacacccacacaactaatgtcattaaatatcatgtcaaaaacttgtacaattattaataaaaataatttagtgtttaaattttaccagttccagattttacacctccgttaaccccactttttacaccactggacgatcctcctccccacattccaccgccaccagatgtataagttttagatcctttattactaccatcatgtccatggataaagacactccacatgccgccactaccccctttagaagacatattaataagacttaaggacaagtttaacaataaaattaatcacgagtaccctactaccaacctacactattatatgattatagtttctatttttacagtaccttaactaaagtctctagtcacaagagcaatactaccaacctacactattatatgattatagtttctatttttataggaacgcgtacgagaaaatcaaatgtctaatttctaacggtagtgttgataaacgattatcgtcaatggatacctcctctatcatgtcgtctattttcttactttgttctattaacttattagcattatatattatttgattataaaacttatattgcttattagcccaatctgtaaatatcggattattaacatatcgtttctttgtaggtttatttaacatgtacatcactgtaagcatgtccgtaccatttattttaatttgacgcatatccgcaatttctttttcgcagtcggttataaattctatatatgatggatacatgctacatgtgtacttataatcgactaatatgaagtacttgatacatattttcagtaacgatttattattaccacctatgaataagtacctgtgatcgtctaggtaatcaactgttttcttaatacattcgatggttggtaatttactcagaataatttccaatatcttaatatataattctgctatttctgggatatatttatctgccagtataacacaaatagtaatacatgtaaacccatattttgttattatattaatgtctgcgccattatctattaaccattctactaggctgacactatgcgacttaatacaatgataaagtatactacatccatgtttatatcatcaatatacggcttacaaagttttagtatcgataacacatccaactcacgcatagagaaggtagggaataatggcataatatttattaggttatcatcattgtcattatctacaactaagtttccattttttaaaatatactcgacaactttaggatctctattgccaaatttttgaaaatatttatttatatgcttaaatctatataatgtagctccttcatcaatcatacatttaataacattgatgtatactgtatgataagatacatattctaacaatagatcttgtatagaatctgtatatcttttaagaattgtggatattattacgtaaactattacacaattctaaaatataaaacgtatcacggtcgaataatagttgatcaactatataattatcgattttgtgatttttcttcctaaactgtttacgtaaatagttagatagaatattcattagttcatgaccactatagttactatcgaataacgcgtcaaatatttcccgtttaatatcgcatttgtcaagataataatagagtgtggtatgttcacgataagtataataacgcatctctttttcgtgtgaaattaaatagtttattacgtccaaagatgtagcataaccatcttgtgacctagtaataatataataatagagaactgttttacccattctatcatcataatcagtggtgtagtcgtaatcgtaattgtctaattcatcatcccaattataatattcaccagcacgtctaatctgttctattttgatcttgtatccatactgtatgttgctacatgtaggtattcctttatccaataatagtttaaacacatctacattgggatttgatgttgtagcgtatttttctacaatattaataccatttttgatactatttatttctatacctttcgaaattagtaatttcaataagtctatatcgatgttatcagaacatagatattcgagtatatcaaaatcattgatatttttatagtcgactgacgacaataacaaaatcacaacatcgtttttgatattattatttttcttggtaacgtatgcctttaatggagtttcaccatcatactcatataatggatttgcaccactttctatcaatgattgtgcactgctggcatcgatgttaaatgttttacaactatcatagagtatcttatcgttaaccatgattggttgttgatgctatcgcattttttggtttctttcatttcagttatgtatggatttagcacgtttgggaagcatgagctcatatgatttcagtactgtagtgtcagtactattagtttcaataagatcaatctctagatctatagaatcaaaacacgataggtcagaagataatgaatatctgtaggcttcttgttgtactgtaacttctcgttttgttagatgtttgcatcgtgctttaacatcaatggtacaaattttatcctcgctttgtgtatcatattcgtccctactataaaattgtatattcagattatcatgagatgtgtatacgctaacggtatcaataaacggagcacaccatttagtcataaccgtaatccaaaaatttttaaagtatatcttaacgaaagaagttgtgtcattgtctacggtgtatggtactagatcctcataagtgtatatatctagagtaatgtttaatttatcaaatggttgataatatggatcctcatgacaatttccgaagatggaaatgagatatagacatgcaataaatctaattgcggacatggttactccttaaaaaaatacgaataatcaccttggctatttagtaagtgtcatttaacactatactcatattaatccatggactcataatctctatacgggattaacggatgttctatatacggggatgagtagttttcttctttaactttatactttttactaatcatatttagactgatgtatgggtaatagtgtttaaagagttcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccatactgaatggatgaacgaataccgacggcgttaatagtaatttactttttcatctttacatattgggtactagttttactatcataagtttataaattccacaagctactatggaataagccaaccatcttagtataacacacatgtcttaaagtttattaattaattacatgttgttttatatatcgctacgaatttaaacagagaaatcagtttaggaaaaaaaaatatctatctacatcatcacgtctctgtattctacgatagagtgctactttaagatgagacatatccgtgtcatcaaaaatatactccattaaaatgattattccggcagcgaacttgatattggatatatcacaacctttgttaatatctacgacaatagacagcagtcccatggttccataaacagtgagtttatctttctttgaagagatattttgtagagatcttataaaactgtcgaatgacatcgcatttatatctttagctaaatcgtatatgttaccatcgtaatatctaaccgcgtctatcttaaacgtttccatcgctttaaagacgtttccgatagatggtctcatttcatcagtcatactgagccaacaaatataatcgtgtataacatctttgatagaatcagactctaaagaaaacgaatcggctttattatacgcattcatgataaacttaatgaaaaatgtttttcgttgtttaagttggatgaatagtatgtcttaataattgttattatttcattaattaatatttagtaacgagtacactctataaaaacgagaatgacataactagttatcaaagtgtctaggacgcgtaattttcatatggtatagatcctgtaagcattgtctgtattctggagctattttctctatatctaatttctgaacgttcaccaatgtctctagccactttggcactaatagcgatcattcgcttagcgtcttctatattattaactggttgattcaatctatctagcaatggaccgtcggacagcgtcattctcatgttcttaatcaatgtacatacatcgccgtcatctaccaattcatccaacaacataagctttttaaaatcatcattataataggtttgatcgttgtcatttctccaaagaatatatctaataagtagagtcctcatgattagttaacaactattttttatgttaaatcaattagtacaccgctatgtttaatacttattcatattttagtttttaggattgagaatcaatacaaaaattaatgcatcattaattttagaaatacttagtttccacgtagtcaatgaaacatttgaactcatcgtacaggacgttctcgtacaggacgtaactataaaccggtttatatttgttcaagatagatacaaatccgataactttttttacgaattctacgggatccactttaaaagtgtcataccgggttctttttatttttttaaacagattaatggtgtgatgttgattaggtcttttacgaatttgatatagaatagcgtttacatattctccataatggtcaatcgccatttgttcgtatgtcataaattctttaattatatgacactgtgtattatttagttcatccttgttcatcattaggaatctatccaatatggcaattatactagaactataggtgcgttgtatacacatattgatgtgtctgtttatacaatccatgctactaccttcgggtaaaattgtagcatcatataccatttctagtactttaggttcattgttatccattgcagaggacgtcatgaacgcatcctaaaaaaatatattatttttatgttattttgttaaaaataatcatcgaatacgaactagtataaaaaggcgcgccttaggcctgagttgagtcagcactgctcatggattgttgcaattgtttggagaaatcatccaaatctgcagcaggaagaagagtcacagtttgctgtttcttctgtctctgcggtaaggcttgagtttcatcagccttcttcttcttgtccttcttaggctctgttggtgggaatgtcttgtatgcgtcaatatgcttattcagcaatatgacttgatctttgaaatttggatctttgtcatccaatttgatggcacctgtgtaggtcaaccacgttcccgaaggtgtgacttccatgccaatgcgcgacattccgaagaacgctgaagcgctgggagcaaattgtgcaatttgcggccaatgtttgtaatcagttccttgtctgattagttcctggtctccaaagtttccttgggtttgttctggaccacgtctgccgaaagcttgtgttacattgtatgctttagtggcagtacgtttctgccgaggcttcttagaagcctcagcagcagatttcttagtgacagtttggccttgttgttgttggcctttaccagacatcttgctctcaagctggttcaatctgtcaagcagcagcaaagcaagagcagcatcaccgccattgccagccattctagcaggagaagttcctctactgctgcctggagttgaatttcttgaactgttgcgactacgtgatgaggaacgagaagaggcttgactgccgcctctgctcccttctgcgtagaagcctttaggcaatgttgttccttgaggaagttgtagcacgattgcagcattgttagcaggattgcgggtgccaatgtgatctttaggtgtattcaaggctccctcagttgcaacccatatgatgccgtctttgttagcaccatagggaagtccagcttctggcccagttcctaggtagtagaaataccatcttggactgagatctttcatcttaccgtcaccaccacgaattcgtctggtagctcttcggtagtagccaatttggtcatctggactgctattggtgttaattggaacgccttgtcctcgagggaatttaaggtcttccttgccatgttgagtgagagcggtgaaccaagacgcagtattattgggtaaaccttgaggccgacgttgtttagatcgcgctccactgcgttctccattctggttactgccagttgaatctgagggtccaccaaacgtaatgcgtggtgcgtttcgctgattctgtggtccgttatcagacatggtggcggcgtttatcttatttatgattatttctcgctttcaatttaacacaaccctcaagaacctttgtatttattttcaatttttgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagagaaagcttttcagctgcttagacttccaagtattaattcgtgacagatccatgtctgaaacgagacgctaattagtgtataatttttgtcatattgcaccagaattaataatatctctaatagatctgattagtagatacatggctatcgcaaaacaacatatacacatttaataaaaataatatttattaagaaaattcagatttcacgtacccatcaatataaataaaataatgattccttacaccgtacccatattaaggagattctaccttacccataaacaatataaatccagtaatatcatgtctgatgatgaacacaaatggtgtattaaattccagtttttcaggagatgatctcgccgtagctaccataatagtagatgcctctgctacagttccttgttcgtcgacatctatctttgcattctgaaacattttataaatatataatgggtccctagtcatatgtttaaacgacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatgtcatattctttgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatacacacagtattatctacgaaactttgataagttagatcagtgtacgtatatttagatgttttcagcttagctaatcctgatattaattctgtaaatgctggacccagatctctttttctcaaatccatagtcttcaataattctattctagtattacctgatgcaggcaatagcgacataaacatagaaaacgaataaccaaacggtgagaagacaatattatcatcttgaatatttttatacgctactataccggcattggtaaatccttgtagacgataggtagacgctgaacacgttaacgatagtatcaataacgcaatcatgattttatggtattaataattaaccttatttttatgttcggtataaaaattattgatgtctacacatccttttgtaattgacatctatatatccttttgtataatcaactctaatcactttaacttttacagttttccctaccagtttatccctatattcaacatatctatccatatgcatcttaacactctctgccaagatagcttcagagtgaggatagtcaaaaagataaatatatagagcataatcattctcgtatactctgccctttattacatcgcccgcattgggcaacgaataacaaaatgcaagcatcttgttaacgggctcgtaaattgggataaaaattatgtttttatatctattttattcaagagaatattcaggaatttctttttccggttgtatctcatcgcagtatatatcatttgtacattgtttcatattttttaatagtttacaccttttagtaggactagtatcgtacaattcatagctgtattttgaattccaatcacgcataaaaatatcttctaattgttgacgaagacctaatccatcatccggtgtaatattaatagatgctccacatgtatccgtaaagtaatttcctgtccaatttgaggtacctatataggccgttttatcggttaccatatatttggcatggtttaccctagaatacggaatgggaggatcagcatctggtacaataaatagctttacttctatatttatgtttttagattttagcatagcgatagatcttaaaaagtttctcatgataaacgaagatcgttgccagcaactaatcaatagcttaacggatacttgtctgtctatagcggatcttcttaattcatcttctatataaggccaaaacaaaattttacccgccttcgaataaataatagggataaagttcataacagatacataaacgaatttactcgcatttctaatacatgacaataaagcggttaaatcattggttctttccatagtacatagttgttgcggtgcagaagcaataaatacagagtgtggaacaccacttacgttaatactaagaggatgatctgtattataatacgacggataaaagtttttccaattatatggtagattgttaactccaagataccagtatacctcaaaaatttgagtgagatccgctgccaagttcctattattgaagatcgcaatacccaattctttgacctgagttagtgatctccaatccatgttagcgcttcctaaataaatatgtgtattatcagatatccaaaattttgtatgaagaactcctcctaggatatttgtaatatctatgtatcgtacttcaactccggccatttgtagtctttcaacatcctttaatggtttgttagatttattgacggctactctaactcgtactcctcttttgggtaattgtacaatctcgtttaatattatcgtgccgaaattcgtacccacttcatccgataaactccaataaaaagatgatatatctagtgtttttgtggtattggatagaatttccctccacatgttaaatgtagacaaatatactttatcaaattgcatacctataggaatagtctctgtaatcactgcgattgtattatccggattcattttatttgttaaaagaataatcctatatcacttcactctattaaaaatccaagtttctatttctttcatgactgattttttaacttcatccgtttccttatgaagatgatgtttggcaccttcataaatttttatttctctattacaatttgcatgttgcatgaaataatatgcacctaaaacatcgctaatctcattgtttgttccctggagtatgagagtcggggtgttaatcttggaaattatttttctaaccttgttggtagccttcaagacctgactagcaaatccagccttaattttttcatgattgattaatgggtcgtattggtatttataaactttatccatatctctagatactgattctggacatagctttccgactggcgcatttggtgtgatggttcccataagtttggcagctagcagattcagtcttgaaacagcatctgcattaactagaggagacattagaatcattgctgtaaacaagtttggattatcgtaagaggctagtatagaaattgttgctcccatggaatgacccaataagtagatttaatagttaccacgtgctgtaccaaagtcatcaatcatcattttttcaccattacttcttccatgtccaatatgatcatgtgagaatactaaaattcctaacgatgatatgttttcagctagttcgtcataacgtccagaatgtttaccagctccatgacttatgaatactaatgccttaggatatgtaatcattgtccagattgaacatacagtttgcactcatgattcacgttatataactatcaatattaacagttcgtttgatgatcatattatttttatgttttattgataattgtaaaaacatacaattaaatcaatatagaggaaggagacggctactgtcttttgtgagatagtcatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattgatgatataggggtcttcataacgcataattattacgttagcattctatatccgtgttaaaaaaaattatcctatcatgtatttgagagttttatatgtagcaaacatgatagctgtgatgccaataagctttagatattcacgcgtgctagtgttagggatggtattatctggtggtgaaatgtccgttatataatctacaaaacaatcatcgcatatagtatgcgatagtagagtaaacatttttatagtttttactggattcatacatcgtctacccaattcggttatgaatgaaattgtcgccaatcttacacccaaccccttgttatccattagtatagtattaacttcgttatttatgtcataaactgtaaatgattttgtagatgccatatcatacatgatattcatgtccctattataatcattactaactttatcacaatatatgttgataatatctatatatgatctagtctttgtgggcaactgtctatacaagtcgtctaaacgttgtttactcatatagtatcgaacagccatcattacatggtcccgttccgttgatagataatcgagtatgttagtggacttgtcaaatctatataccatattttctggaagtggatatacatagtcgtgatcaacattattgctagcctcatcttctatatcctgtactataccatctacataatctacgatattattacacataaacatcgacaacatactattgtttattatctaagtcctgttgatccaaacccttgatctcctctatttgtactatctagagattgtacttcttccagttctggataatatatacgttgatagattagctgagctattctatctccagtatttacattaaacgtacattttccattattaataagaatgactcctatgtttcccctataatcttcgtctattacaccacctcctatatcaatgccttttagtgacagaccagacctaggagctattctaccatagcaaatcttaggcatggacatactaatatctgtcttaattaactgtctttctcctggagggatagtataatcgtaagcgctatacaaatcatatccggcagcacccggcgattgcctagtaggagatttagctctgttagtttccttaacaaatctaactggtgagttaatattcatgttgaacataaaactaatattttatttcaaaattatttaccatcccatatattccatgaataagtgtgatgattgtacacttctatagtatctatatacgattcacgataaaatcctcctatcaatagcagtttattatccactatgatcaattctggattatccctcggataaataggatcatctatcagagtccatgtattgctggattcacaataaaattccgcatttctaccaaccaagaataaccttctaccgaacactaacgcgcatgatttataatgaggataataagtggatggtccaaactgccactgatcatgattgggtagcaaatattctgtagttgtatcagtttcagaatgtcctcccattacgtatataacattgtttatggatgccactgctggattacatctaggtttcagaagactcggcatattaacccaagcagcatccccgtggaaccaacgctcaacagatgtgggatttggtagacctcctactacgtataatttattgttagcgggtatcccgctagcatacagtctggggctattcatcggaggaattggaatccaattgtttgatatataatttacagctatagcattgttatgtatttcattgttcatccatccaccgatgagatatactacttctccaacatgagtacttgtacacatatggaatatatctataatttgatccatgttcataggatactctatgaatggatacttgtatgatttgcgtggttgtttatcacaatgaaatattttggtacagtctagtatccattttacattatttatacctctgggagaaagataatttgacctgattacatttttgataaggagtagcagatttcctaatttatttcttcgctttatataccacttaatgacaaaatcctcatctggaacatttagttcatcgctttctagaataagtttcatagatagataatcaaaattgtctatgatgtcatcttccagttccaaaaagtgtttggcaataaagtttttagtatgacataagagattggatagtccgtattctatacccatcatgtaacactcgacacaatattcctttctaaaatctcgtaggataaagtttatacaagtgtagatgataaattctacagaggttaatatagaagcacgtaataaattgacgacgttatgactatctatatatacctttccagtatatgagtaaataactatagaagttagactgtgaatgtcaaggtctagacaaaccctcgtaactggatctttatttttcgtgtatttttgacgtaaatgtgtgcgaaagtaaggagataactttttcaatatcgtagaattgactattatattgcctcctatggcatcaataattgttttgaatttcttagtcatagacaatgctaatatattcttacagtacacagtattgacaaatatcggcatttatgtttctttaaaagtcaacatctaaagaaaaatgattatcttcttgagacataactcccattttttggtattcacccacacgtttttcgaaaaaattagttttaccttctaatgatatattttccatgaaatcaaacggattggtaacattataaatttttttaaatcccaattcagaaatcaatctatccgcgacgaattctatatatgttttcatcatttcacaattcattcctataagtttaactggaagagccgcagtaagaaattcttgttcaatggataccgcatctgttataatagatctaacggtttcttcactcggtggatgcaataaatgtttaaacatcaaacatgcgaagtcgcagtgtagaccctcgtctctactaatcaattcgttggaaaacgtgagtccgggcattaggccacgctttttaagccaaaatatggaagcgaatgatccggaaaagaagattccttctactgcagcaaaggcaataagtctctctccataaccggcgctgtcatgtatccacttttgagcccaatcggccttcttttttacacaaggcatcgtttctatggcattaaagagatagtttttttcattactatctttaacataagtatcgatcaaaagactatacatttccgaatgaatgttttcaatggccatctgaaatccgtagaaacatctagcctcggtaatctgtacttctgtacaaaatcgttccgccaaattttcattcactattccgtcactggctgcaaaaaacgccaatacatgttttataaaatatttttcgtctggtgttagtttattccaatcattgatatctttagatatatctacttcttccactgtccaaaatgatgcctctgcctttttatacatgttccagatgtcatgatattggattgggaaaataacaaatctatttggatttggtgcaaggatgggttccataactaaattaacaataacaataaattttttttcagttatctatatgcctgtacttggatcttttgtacatcgatatcgccgcaatcactacaataattacaagtattattgatagcattgttattagtactatcataattaaattatcgttattatcattttgtaattgtgacatcatactagataaatcgtttgcgagattgttgtgggaagcgggcatggaggatgaattatcgttattattatttaaagcctcccattcggattcacaaatatggcgcgcgttcaacattttatggaaacagataacaagaaaactcgtcatcgttcaaatttttaacgatagtaaaccgattaaacgtcgagctaatttctaacgctagcgactctgttggatatgggtttccagatatatatcttttcagttcccctacgtatctataatcatctgtaggaaatggaagatatttccatttatctactgttcctaatatcatatgtggtggtgtagtagaaccattaagcgcgaaagatgttatttcgcatcgtattttaacttcgcaataatttctggttagataacgcactctaccagtcaagtcaatgatattagcctttacagatatattcatagtagtcgtaacgatgactccatcttttagatgcgatactcctttgtatgtaccagaatcttcgtacctcaaactcgatatatttaaacaagttaatgagatattaacgcgttttatgaatgatgatatataaccagaagttttatcctcggtggctagcgctataaccttatcattataataccaactagtgtgattaatatgtgacacgttagtgtgggtacaaatatgtacattatcgtctacgtcgtattcgatacatccgcatacagccaacaaatataaaatgacaaatactctaacgccgttcgtacccatcttgatgcggtttaataaatgttttgatttcaatttattgtaaaaaaagattcggttttatactgttcgatattctcattgcttatattttcatctatcatctccacacagtcaaatccgtggttagcatgcacctcatcaaccggtaaaagactatcggactcttctatcattataactctagaatatttaatttggtcattattaatcaagtcaattatcttatttttaacaaacgtgagtattttactcattttttataaaaacttttagaaatatacagactctatcgtgtgtctatatcttctttttatatccaatgtatttatgtctgatttttcttcatttatcatatataatggtccaaattctacacgtgcttcggattcatccagatcattaaggttcttataattgtaacatccttctcttccctcttctacatcttccttcttattcttattcttagcgtcacagaatctaccacagcaggatcccatgacgagcgtcatattaaactaatccattttcaattataatatacgattagtaatgaccattaaaataaaaatattcttcataaccggcaagaaagtgaaaagttcacattgaaactatgtcagtagtatacatcatgaaatgatgatatatatatactctattttggtggaggattatatgatataattcgtggataatcattcttaagacacatttcttcattcgtaaatcttttcacgttaaatgagtgtccatattttgcaatttcttcatatgatggcggtgtacgtggacgaggctgctcctgttcttgttgtagtcgccgactgtcgtgtctgcgtttagatccctccattatcgcgattgcgtagatggagtactattttataccttgtaattaaatttttttattaattaaacgtataaaaacgttccgtatctgtatttaagagccagatttcgtctaatagaacaaatagctacagtaaaaataactagaataattgctacacccactagaaaccacggatcgtaatacggcaatcggttttcgataataggtggaacgtatattttatttaaggacttaacaattgtctgtaaaccacaatttgcttccgcggatcctgtattaactatctgtaaaagcatatgttgaccgggcggagccgaacattctccgatatccaatttctgtatatctataatattattaacctccgcatacgcattacagttcttttctagcttggataccgcactaggtacatcgtctagatctattcctatttcctcagcgatagctcttctatccttttccggaagcaatgaaatcacttcaataaatgattcaaccatgagtgtgaaactaagtcgagaattactcatgcatttgttagttattcggagcgcgcaatttttaaactgtcctataacctctcctatatgaatagcacaagtgacattagtagggatagaatgttgagctaatttttgtaaataactatctataaaaagattatacaaagttttaaactctttagtttccgccatttatccagtctgagaaaatgtctctcataataaatttttccaagaaactaattgggtgaagaatggaaacctttaatctatatttatcacagtctgttttggtacacatgatgaattcttccaatgccgtactaaattcgatatctttttcgatttctggatatgtttttaataaagtatgaacaaagaaatggaaatcgtaataccagttatgtttaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcagcatggtagttcgtaaattttaatgtataacagagccatctgtaaaaagataaactttatgtattgtaccaaagatttaaatcctaatttgatagctagctcggtatctactttatctgccgaatacagtgctaggggaaaaattataatatttcctctttcgtattcgtagttagttctcttttcatgttcgaaaaagtgaaacatgcggttaaaatagtttataacattaatattactgttaataactgccggataaaagtgggatagtaatttcacgaatttgatactgtcctttctctcgttaaacgcctttaaaaaaactttagaagaatatctcaatgatagttcctgaccatccatagtttgtatcaataatagcaacatatgaagaacacgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccttattaaaattcttaaaataatttaacaatctaactgatggagcccaattttggtgtaaatctaattgggacattatattgttaaaatacaaacagtctcctaatataacagtatctgataatctatggggagacatccattgatattcaggggatgaatcattggcaacacccatttattgtacaaaaagccccaatttacaaacgaaagtccaggtttgatagagacaaactattaactattttgtctctgtttttaatttctttggtaatgaaattattcacaatatcagtatcttctttatctaccagagattttactaacttgataaccttggctgtctcattcaatagggtagtaatatttgtatgtgtgatattgatatcttttagaagtgattctttgatggtgccagcatacgaattacaataatgcagaaactcggttaacatgcaggaattatagtaagccaattccaattgttgcctgtgttgtattagagtgtcaatatgagcaatggtgtccttgcgtttctctgatagaatgcgagcagcgattttggcgttatcatttgacgatatttctggaatgacgaatcctgtttctactaactttttggtaggacaaagtgaaacaatcaagaagatagcttctcctcctatttgtggaagaaattgaactcctctagatgatctccttgacagatattggaccgaattacagaagtacctggaatgtaaagccctgaaaccccctcattttttaagcagattgttgccgtaaatcctgcactatgcccaagatagagagctcctttggtgaatccatctctatgtttcagtttaaccaagaaacagtcagctggtctaaaatttccatctctatctaatacagcatctaacttgatgtcaggaactatgaccggttatgttatatgtaacattgagtaaatccttaagttcataatcatcactgtcatcagttatgtacgatccaaacaatgtttctactggcatagtggatacgaagatgctatccatcagaatgtttccctgattagtattttctatatagctattcttctttaaacgattttccaaatcagtaactatgttcatttttttaggagtaggacgcctagccagtatggaagaggattttctagatcctctcttcaacatctttgatctcaatggaatgcaaaaccccatagtgtaacaaccaacgataaaaataatattgtttttcactttttataattttaccatctgactcatggattcattaatatctttataagagctactaacgtataattctttataactgaactgagatatatacaccggatctatggtttccataattgagtaaatgaatgctcggcaataactaatggcaaatgtataaaacaacgaaattatactagagttgttaaagttaatattttctatgagctgttccaataaattatttgttgtaactgcgttcaagtcataaatcatcttgatactatccagtaaaccgtttttaagttctggaatattattatcccattgtaaagcccctaattcgactatcgaatatcctgctctgatagcagtttcaatatcgacggacgtcaatactgtaataaaggtggtagtattgtcatcatcgtgataaactactggaatatggtcgttagtaggtacggtaactttacacaacgcgatatataactttccttttgtaccatttttaacgtagttgggacgtcctgcagggtattgttttgaagaaatgatatcgagaacagatttgatacgatatttgttggattcctgattatttactataatataatctagacagatagatgattcgataaatagagaaggtatatcgttggtaggataatacatccccattccagtattctcggatactctattaatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacagttattgggccagtatactgttagcaacggtatatcgaatagattactcatgtaacctactagaatgatagttcgtgtactagtcataatatctttaatccaatctaagaaatttaaaattagattttttacactgttaaagttaacaaaggtattacccggatacgtggatatcatatatggtattggtccattatcagtaatagctccataaactgatacggcgatggtttttatatgtgtttgatctaacgaggaagaaattcgcgcccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctataatcgttaaattttacaaatctataacatgctaaaccttttgacgacaaccattcattaatttctgatatggaatctgtattctcaataccgtatcgttctaaagccagtgctatatctccctgttcgtgagaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatattgtaatattcatcatcgacataacactatctatgttatcatcgtatattagtataccatgaccttcttcatttcgtgccaaaatgatatacagtcttaaatagttacgcaatatctcaatagtttcataattgttagctgttttcatcaaggtttgtatcctgtttaacatgatggcgttctatacgtttctattttttaaatttttaacgatttactgtggctagatacccaatctctctcaaatatttttttagcctcgcttacaagctgtttatctatactattaaaactgacgaatccgtgattttggtaatgggttccgtcgaaatttgccgaagtgatatgaacatattcgtcgtcgactatcaacaattttgtattattctgaatagtgaaaaccttcacagatagatcattttgaacacacaacgcgtctagacttctggcggttgccatagaatatacgtcgttcttatcccaattaccaactagaagtctgatcttaactcctctattaatggctgcttctataatggagttgtaaatgtcgggccaatagtagctattaccgtcgacacgtgtagtgggaactatggccaaatgttcaatatctatactagtcttagccgacttgagtttatcaataactacatcagtgtctagatctctagaatatcccaataggtgttccggagaatcagtaaagaacactccacctataggattcttaatatgatacgcagtgctaactggcagacaacaagccgcagagcataaattcaaccatgaattttttgcgctattaaaggctttaaaagtatcaaatcttctacgaagatctgtggccagcgggggataatcagaatatacacctaacgttttaatcgtatgtatagatcctccagtaaatgacgcgtttcctacataacatctttcattatctgacacccaaaaacaaccgagtagtagtcccacattattttttttatctatattaacggttataaaatttatatccgggcagtgactttgtagctctcccagatttcttttccctcgttcatctagcaaaactattattttaatccctttttcagatgcctcttttagtttatcaaaaataagcgctcccctagtcgtactcagaggattacaacaaaaagatgctatgtatatatatttcttagctagagtgataatttcgttaaaacattcaaatgttgttaaatgatcggatctaaaatccatattttctggtagtgtttctaccagcctacattttgctcccgcaggtaccggtgcaaatggccacatttagttaacataaaaacttatacatcctgttctatcaacgattctagaatatcatcggctatatcgctaaaattttcatcaaagtcgacatcacaacctaactcagtcaatatattaagaagttccatgatgtcatcttcgtctatttctatatccgtatccattgtagattgttgaccgattatcgagtttaaatcattactaatactcaatccttcagaatacaatctgtgtttcattgtaaatttataggcggtgtatttaagttggtagattttcaattatgtatcaatatagcaacagtagttcttgctcctccttgattctagcatcctcttcattattttcttctacgtacataaacatgtccaatacgttagacaacacaccgacgatggcggccgccacagacacgaatatgactaaaccgatgaccatttaaaaacccctctctagctttcacttaaactgtatcgattattcttttagaacatgtataatataaaaacattattctatttcgaatttaggcttccaaaaatttttcatccgtaaaccgataataatatatatagacttgttaatagtcggaataaatagattaatgcttaaactatcatcatctccacgattagagatacaatatttacattttttttgctgtttcgaaactttatcaatacacgttaatacaaacccaggaaggagatattgaaactgaggctgttgaaaatgaaacggtgaatacaataattcagataatgtaaaatcatgattccgtattctgatgatattagaactgctaatggatgtcgatggtatgtatctaggagtatctattttaacaaagcatcgatttgctaatatacaattatcattttgattaattgttattttattcatattcttaaaaggtttcatatttatcaattcttctacattaaaaatttccatttttaatttatgtagccccgcaatactcctcattacgtttcattttttgtctataatatccattttgttcatctcggtacatagattatccaattgagaagcgcatttagtagttttgtacattttaagtttattgacgaatcgtcgaaaactagttatagttaacattttattatttgataccctgatattaatacccctgccgttactattatttataactgatgtaatccacgtaacattggaattaactatcgatagtaatgcatcgacgcttccaaaattgtctattataaactcaccgataatttttttattacatgttttcatattcattaggattattaaatctttaatcttactacgattgtatgcgttgatattgcaagacgtcattctaaaagacggaggatctccatcaaatgccagacaatcacgtacaaagtacatggaaataggttttgttctattgcgcatcatagatttatatagaacacccgtagaaatactaatttgttttactctataaaatactaatgcatctatttcatcgttttgtataacgtctttccaagtgtcaaattccaaatttttttcattgatagtaccaaattcttctatctctttaactacttgcatagataggtaattacagtgatgcctacatgccgttttttgaaactgaatagatgcgtctagaagcgatgctacgctagtcacaatcaccactttcatatttagaatatatatatgtaaaaatatagtagaatttcattttgttttttttctatgctataaatgaattctcattttgcatctgctcatactccgttttatatcaataccaaagaaggaagatatctggttctaaaagccgttaaagtatgcgatgttagaactgtagaatgcgaaggaagtaaagcttcctgcgtactcaaagtagataaaccctcatcacccgcgtgtgagagaagaccttcgtccccttccagatgcgagagaatgaataacccaggaaaacaagttccgtttatgaggacggacatgctacaaaatatgttcgcggctaatcgcgataatgtagcttctagacttttgtcctaaaatacaattatatccttttcgatattaataaatccgtgtcgtccaggttttttatctctttcagtatgtgaatagataggtattttatctctattcatcatcgaatttaagagatccgataaacattgtttgtattctccagatgtcagcatctgatacaacaatatatgtgcacataaacctctggcacttatttcatgtaccttccccttatcactaaggagaatagtatttgagaaatatgtatacatgatattatcatgaattagatatacagaatttgtaacactctcgaaatcacacgatgtgtcggcgttaagatctaatatatcactcgataacacattttcatctagatacactagacattttttaaagctaaaatagtctttagtagtaacagtaactatgcgattattttcatcgatgatacatttcatcggcatattattacgcttaccatcaaagactataccatgtgtatatctaacgtattctagcatggttgccatacgcgcattaaacttttcaggatctttggatagatcttccaatctatctatttgagaaaacatttttatcatgttcaatagttgaaacgtcggatccactatatagatattatctataaagattttaggaactacgttcatggtatcctggcgaatattaaaactatcaatgatatgattatcgttttcatcttttatcaccatatagtttctaagatatgggattttacttaatataatattatttcccgtaataaattttattagaaatgccaaatctataagaaaagtcctcgaattagtttgaagaatatctatatcgccgtaccgtatatttggattaattagatatagagaatatgatccgtaacatatacaacttttattatggcgtctaagatattcttccatcaacttattaacatttttgactagggaagatacattatgacgtcccattacttttgccttgtctattactgcgacgttcatagaatttagcatatctcttgccaattcttccattgatgttacattataagaaattttagatgaaattacatttggagctttaatagtaagaactcctaatatgtccgtgtatgtggtcactaatacagattgtagttctataatcgtaaataatttacctatattatatgtttgagtctgtttagaaaagtagctaagtatacgatcttttatttctgatgcagatgtatcaacatcggaaaaaaatctttttttattcttttttactaaagatacaaatatgtctttgttaaaaacagttattttctgaatatttctagcttgtaattttaacatatgatattcgttcacactaggtactctgcctaaataggtttctataatctttaatgtaatattaggaaaagtattctgatcaggattcctattcattttgaggatttaaaactctgattattgtctaatatggtctcaacacaaactttttcacagagcgatagagtttttgataactcgtttttcttaagaaatataaaactactgtctccagagctcgctctatcttttattttatttaattcgatacaaactcctgatactggttcagaaagtaattcattaattttcagtcctttatagaagatatttaatatagataatacaaaatcttcagtttttgatatcgatctgattgatcctagaactagatatattaataacgtgctcattaggcagtttatggcagcttgataattagatatagtatattccagttcatatttattagataccgcattgcccagattttgatattctatgaattcctctgaaaataaatccaaaataactagacattctattttttgtggattagtgtactctcttccctctatcatgttcactactggtgtccacgatgataaatatctagagggaatataatatagtccataggatgccaatctagcaatgtcgaataactgtaattttattcttcgctcttcattatgaattgattcttgaggtataaacctaacacaaattatattattagacttttcgtatgtaatgtctttcatgttataagtttttaatcctggaatagaatctattttaatgaggcttttaaacgcagagttctccaacgagtcaaagcataatactctgttggttttcttatatacgatgttacgattttcttctttgaatggaataggtttttgaattagtttataattacaacataatagataaggaagtgtgcaaatagtacgcggaaaaaacataatagctcccctgttttcatccatggttttaagtaaatgatcactggcttctttagtcaatggatattcgaacattaaccgtttcatcatcattggacagaatccatatttcttaatgtaaagagtgatcaaatcattgtgtttattgtaccatcttgttgtaaatgtgtattcggttatcggatctgctcctttttctattaaagtatcgatgtcgatctcgtctaagaattcaactatatcgacatatttcatttgtatacacataaccattactaacgtagaatgtataggaagagatgtaacgggaacagggtttgttgattcgcaaactattctaatacataattcttctgttaatacgtcttgcacgtaatctattatagatgccaagatatctatataattattttgtaagatgatgttaactatgtgatctatataagtagtgtaataattcatgtatttcgatatatgttccaactctgtctttgtgatgtctagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacctgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgataactagaatcagaatctaatgatgacgtaaccaagaagtttatctactgccaatttagctgcattatttttagcatctcgtttagattttccatctgccttatcgaatactcttccgtcgatatctacacaggcataaaatgtaggagagttactaggccccactgattcaatacgaaaagaccaatctctcttagttatttggcagtactcattaataatggtgacagggttagcatctttccaatcaataatttttttagccggaataacatcatcaaaagacttatgatcctctctcattgatttttcgcgggatacatcatctattatggcgtcagccataacatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatgtttttaatagccgcacacacaatctctgcgtcagaacgctcgtcaatatagatcttagacatttttagagagaactaacacaaccagcaataaaactaatttattttatcatttttttattcatcatcctctggtggttcgtcgtttctatcgaatgtggatctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcatgtctaactagtggaggttcgtcagcggctctagtttgaatcatcatcggcgtagtattcctacttttacagttaggacacggtgtattgtatttctcgtcgagaacgttaaaataatcgttgtaactcacatcctttattttatctatattgtattctactcctttcttaatgcattttataccgaataagagatagcgaaggaattctttttcggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggctaccgccattgatgtttgttcattggatgagtaactactaatgtatacattttccatttataacacttatgtattaactttgttcatttatattttttcattattatgttgatattaacaaaagtgaatatatatgttaataattgtattgtggttatacggctacaatttcataatgagtggaagtcagtgtccgatgatcaatgacgatagctttactctgaaaagaaagtatcaaatcgatagtgcggagtcaacaataaaaatggataagaagaggataaagtttcagaatagagccaaaatggtaaaagaaataaatcagacaataagagcagcacaaactcattacgagacattgaaactaggatacataaaatttaagagaatgattaggactactactctagaagatatagcaccatctattccaaataatcagaaaacttataaactattctcggacatttcagccatcggcaaagcatcacagaatccgagtaagatggtatatgctctgctgctttacatgtttcccaatttgtttggagatgatcatagattcattcgttatagaatgcatccaatgagtaaaatcaaacacaagatcttctctcctttcaaacttaatcttattagaatattagtggaagaaagattctataataatgaatgcagatctaataaatggagaataattggaacacaagttgataaaatgttgatagctgaatctgataaatatacaatagatgcaaggtataacctaaaacccatgtatagaatcaagggaaaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaaaaatcttcaagagtgcattgactttgttagtgaataggcatttcatctttctccaatactaattcaaattgttaaattaataatggatagtataaatagttattagtgataaaatagtaaaaataattattagaataagagtgtagtatcatagataactctcttctataaaaatggattttattcgtagaaagtatcttatatacacagtagaaaataatatagattttttaaaggatgatacattaagtaaagtaaacaattttaccctcaatcatgtactagctctcaagtatctagttagcaattttcctcaacacgttattactaaggatgtattagctaataccaatttttttgttttcatacatatggtacgatgttgtaaagtgtacgaagcggttttacgacacgcatttgatgcacccacgttgtacgttaaagcattgactaagaattatttatcgtttagtaacgcaatacaatcgtacaaggaaaccgtgcataaactaacacaagatgaaaaatttttagaggttgccgaatacatggacgaattaggagaacttataggcgtaaattatgacttagttcttaatccattatttcacggaggggaacccatcaaagatatggaaatcatttttttaaaactgtttaagaaaacagacttcaaagttgttaaaaaattaagtgttataagattacttatttgggcttacctaagcaagaaagatacaggcatagagtttgcggataatgatagacaagatatatacactctatttcaacaaactggtagaatagtccatagcaatctaacagaaacgtttagagattatatctttcccggagataagactagctattgggtgtggttaaacgaaagtatagctaatgatgcggatattgttcttaatagacacgccattaccatgtatgataaaattcttagttatatatactctgagataaaacaaggacgcgttaataaaaacatgcttaagttagtttatatctttgagcctgaaaaagatatcagagaacttctgctagaaatcatatatgatattcctggagatatcctatctattattgatgcaaaaaacgacgattggaaaaaatattttattagtttttataaagctaattttattaacggtaatacatttattagtgatagaacgtttaacgaggacttattcagagttgttgttcaaatagatcccgaatatttcgataatgaacgaattatgtctttattctctacgagtgctgcggacattaaacgatttgatgagttagatattaataacagttatatatctaatataatttatgaggtgaacgatatcacattagatacaatggatgatatgaagaagtgtcaaatctttaacgaggatacgtcgtattatgttaaggaatacaatacatacctgtttttgcacgagtcggatcccatggtcatagagaacggaatactaaagaaactgtcatctataaaatccaagagtagacggctgaacttgtttagcaaaaacattttaaaatattatttagacggacaattggctcgtctaggtcttgtgttagatgattataaaggagacttgttagttaaaatgataaaccatcttaagtctgtggaggatgtatccgcattcgttcgattttctacagataaaaaccctagtattcttccatcgctaatcaaaactattttagctagttataatatttccatcatcgtcttatttcaaaggtttttgagagataatctatatcatgtagaagaattcttggataaaagcatccatctaaccaagacggataagaaatatatacttcaattgataagacacggtagatcatagaacagaccaaatatattattaataatttgtatatacatagatataattatcacacatttttgataaatgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcagagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataagtataatcccattctaatactttaacctgatgtattacctgcatcttattagaatattaacctaactaaaagacataacatagttgataaaaagcggtaggatataaatattatggctgccaccgttccgcgttttgacgacgtgtacaaaaatgcacaaagaagaattctagatcaagaaacattttttagtagaggtctaagtagaccgttaatgaaaaacacatatctatttgataattacgcgtatggatggataccagaaactgcaatttggagtagtagatacgcaaacttagatgcaagtgactattatcccatttcgttgggattacttaaaaagttcgagtttctcatgtctctatataaaggtcctattcccgtatatgaagaaaaagtaaatactgaattcattgctaatggatcgttctctggtagatacgtatcatatcttcgaaagttttctgcccttccaacaaacgagtttattagttttttgttactgacttccattccaatctataatatcttgttctggtttaaaaatactcagtttgatattactaaacacacattattcagatacgtctatacagataatgccaaacacctggcgttggctaggtatatgcatcaaacaggagactataagcctttgtttagtcgtctcaaagagaattatatatttaccggtcccgttccaataagtatcaaagatatagatcaccctaatcttagtagagcaagaagtccatccgattatgagacattagctaatattagtactatattgtactttaccaagtatgatccggtattaatgtttttattgttttacgtacctgggtattcaattactacaaaaattactccagccgtagaatatctaatggataaactgaatctaacaaagagcgacgtacaactgttgtaaattattttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtttcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgctgagtatttcatcgttgtatatttcttcttcgattgcataatcagattcttatataccgcctcaaactctattttaaaattattaaacaatactctattattaatcagtcgttctaactctttcgctatttctatagacttatcgacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttagcatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacatgatagtacgactttaaatccattttcccaaatatctttacgaattactttacatataatatcctcatcaacagtcacataattacctgtggttaaaacctttgcaaatgcagcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacgtagtcgaacgttagttccaacaattgtttagctattcgtaacaaaactatttcagaacatagaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtaactacgtaataaaagtaatgaggaaatcgtataaatacggtctcgccatttcgacatctggatttcagatataaaaatctgttttcaccgtgactttcaaaccaattaatgcaccgaacatccatttatagaatttagaaatatattttcatttaaatgaatcccaaacattggggaagagccgtatggaccattatttttatagtactttcgcaagcgggtttagacggcaacatagaagcgtgtaaacgaaaactatatactatagtcagcactcttccatgtcctgcatgtagacggcacgcgactatcgctatagaggacaataatgtcatgtctagcgatgatctgaattatatttattattttttcatcagattatttaacaatttggcatctgatcccaaatacgcgatcgatgtgacaaaggttaaccctttataaacttaacccattataaaacttatgattagtcacgactgaaataaccgcgtgattattttttggtataattctacacggcatggtttctgtaactatgaattcaacccccgttacattagtgaaatctttaacaaacagcaagggttcgtcaaagacataaaactcattgtttacaatcgaaatagaccccctatcacacttaaaataaaaaatatccttatcctttaccaccaaataaaattctgattggtcaatgtgaatgtattcacttaacagttccacaaatttatttattaactccgaggcacatacatcgtcggtattttttatggcaaactttactcttccagcatccgtttctaaaaaaatattaacgagttccatttatatcatccaatattattgaaatgacgttgatggacagatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgtccaagactgtcaatctataatagatagtagctataatatattctatgatggtattgaagaagatgacaatcttggcatattgatcatttaacacagacatggtatcaacagatagcttgaatgaaagagaatcagtaattggaataagcgtcttctcgatagagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcatctgtgcgtagatcacatcgtacactttttaattctctatagaagagcgacagacagcaatttctttattctctacagatgtaagatacttgaagacattcctatgatgatgcagaattttggataacacggtattgatggtatctgttaccataattcctttgatggctgatagtgtcagagcacaagatttccaatctttgttttgatatctatatcagacagcatggtgcgtctgacaacacaaggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatctacatcagttttgtattccaaattaaacatggcaacggatttaattttatattcctctattaagtcctcgtcgataataacagaatgtagataatcatttaatccatcgtacatggttggaagatgcttgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagcgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagattttaggtactcatctactctacaagaattttggagagccttaacgatatcagtgacgtttattatttcaggaggaaagaatctaacattgagaatatcggaattaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacatgtcggcgacatgattaagtattgttttttcattattttctcaacaagttctcaataccccaatagatgatagaatatcacccaatgcgtccatgttgtctatttccaacaggtcgctatatccaccaatagaagtttttccaaaaaagattctaggaacagttctaccaccagtaatttgttcaaaatagtcacgcaattcattttcgggtttaaattctttaatatcgacaatttcatacgctcctcttttgaaactaaacttatttagaatatccagtgcatttctacaaaaaggacatgtatacttgacaaaaattgtcactttgttattggccaacctttgttgtacaaattcctcggccattttaatatttaagtgatataaaactatctcgacttatttaactctttagtcgagatatatggacgcagatagctatatgatagccaactacagaaggcaaacgctataaaaaacataattacgacgagcatatttataaatatttttattcagcattacttgatatagtaatattaggcacagtcaaacattcaaccactctcgatacattaactctctcattttctttaacaaattctacaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatctgtcaaaaccaattcctttttgcctgtatcatccagttttccatcctttgtaaagaaattattttctactagactattaataagactgataaggattcctccataattgcacaatccaaactttttaacaaaactagactttacaagatctacaggaatgcgtaattcaggtttcttagcttgtgattttttcttttgtggacattttcttgtgaccaactcatctaccatttcattgattttagcagtgaaataagctttcaatgcacgggcactgatactattgaaaacgagttgatcttcaaattccgccatttaagttcaccaaacaacttttaaatacaaatatatcaatagtagtagaataagaactataaaaaaaataataattaaccaataccaaccccaacaaccggtattattagttgatgtgactgttttctcatcacttagaacagatttaacaatttctataaagtctgtcaaatcatcttccggagaccccataaatacaccaaatatagcggcgtacaacttatccatttatacattgaatattggcttttctttatcgctatcttcatcatattcatcatcaatatcaacaagtcccagattacgagccagatcttcttctacattttcagtcattgatacacgttcactatctccagagagtccgataacgttagccaccacttctctatcaatgattagtttcttgagtgcgaatgtaatttttgtttccgttccggatctatagaagacgataggtgtgataattgccttggccaattgtctttctcttttactgagtgattctagttcaccttctatagatctgagaatggatgattctccagtcgaaacatattctaccatggatccgtttaatttgttgatgaagatggattcatccttaaatgttttctctgtaatagtttccaccgaaagactatgcaaagaatttggaatgcgttccttgtgcttaatgtttccatagacggcttctagaagttgatacaacataggactagccgcggtaacttttatttttagaaagtatccatcgcttctatcttgtttagatttatttttataaagtttagtctctccttccaacataataaaagtggaagtcatttgactagataaactatcagtaagttttatagagatagacgaacaattagcgtattgagaagcatttagtgtaacgtattcgatacattttgcattagatttactaatcgattttgcatactctataacacccgcacaagtctgtagagaatcgctagatgcagtaggtcttggtgaagtttcaactctcttcttgattaccttactcatgattaaacctaaataattgtactttgtaatataatgatatatattttcactttatctcatttgagaataaaaatgtttttgtttaaccactgcatgatgtacagatttcggaatcgcaaaccaccagtggttttattttatccttgtccaatgtgaattgaatgggagcggatgcgggtttcgtacgtagatagtacattcccgtttttagaccgagactccatccgtaaaaatgcatactcgttagtttggaataactcggatctgctatatggatattcatagattgactttgatcgatgaaggctcccctgtctgcagccatttttatgatcgtcttttgtggaatttcccaaatagttttataaactcgcttaatatcttctggaaggtttgtattctgaatggatccaccatctgccataatcctattcttgatctcatcattccataattttctctcggttaaaactctaaggagatgcggattaactacttgaaattctccagacaatactctccgagtgtaaatattactggtatacggttccaccgactcattatttcccaaaatttgagcagttgatgcagtcggcataggtgccaccaataaactatttctaagaccgtatgttctgattttatcttttagaggttcccaattccaaagatccgacggtacaacattccaaagatcatattgtagaataccgttactggcgtacgatcctacatatgtatcgtatggtccttccttctcagctagttcacaactcgcctctaatgcaccgtaataaatggtttcgaagatcttcttatttagatcttgtgcttccaggctatcaaatggataatttaagagaataaacgcgtccgctaatccttgaacaccaataccgataggtctatgtctcttattagagatttcagcttctggaataggataataattaatatctataattttattgagatttctgacaattactttgaccacatccttcagtttgagaaaatcaaatcgcccatctattacaaacatgttcaaggcaacagatgccagattacaaacggctacctcattagcatccgcatattgtattatctcagtgcaaagattactacacttgatagttcctaaattttgttgattactctttttgttacacgcatccttataaagaatgaatggagtaccagtttcaatctgagattctataatcgctttccagacgactcgagcctttattatagatttgtatctcctttctctttcgtatagtgtatacaatcgttcgaactcgtctccccaaacattgtccaatccaggacattcatccggacacatcaacgaccactctccgtcatccttcactcgtttcataaagagatcaggaatccaaagagctataaatagatctctggttctatgttcctcgtttcctgtattctttttaagatcgaggaacgccataatatcagaatgccacggttccaagtatatggccataactccaggccgtttgtttcctccctgatctatgtatctagcggtgttattataaactctcaacattggaataataccgtttgatataccattggtaccggagatatagcttccactggcacgaatattactaattgatagacctattccccctgccattttagagattaatgcgcatcgttttaacgtgtcatagataccctctatgctatcatcgatcatgttaagtagaaaacagctagacatttggtgacgactagttcccgcattaaataaggtaggagaagcgtgcgtaaaccatttttcagaaagtagattgtacgtctcaatagctgagtctatatcccattgatgaattcctactgcgacacgcattaacatgtgctgaggtctttcaacgatcttgttgtttattttcaacaagtaggatttttccaaagttttaaaaccaaaatagttgtatgaaaagtctcgttcgtaaataataaccgagttgagtttatccttatatttgttaactatatccatggtgatacttgaaataatcggagaatgtttcccatttttaggattaacatagttgaataaatcctccatcacttcactaaatagtttttttgtttccttgtgtagatttgatacggctattctggcggctagaatggcataatccggatgttgtgtagtacaagtggctgctatttcggctgccagagtgtccaattctaccgttgttactccattatatattccttgaataaccttcatagctattttaataggatctatatgatccgtgtttaagccataacataattttctaatacgagacgtgattttatcaaacatgacattttccttgtatccatttcgtttaatgacaaacatttttgttggtgtaataaaaaaaattatttaacttttcattaatagggatttgacgtatgtagcgtacaaaattatcgttcctggtatatagataaagagtcctatatatttgaaaatcgttacggctcgattaaactttaatgattgcatagtgaatatatcattaggatttaactccttgactatcatggcggcgccagaaattaccatcaaaagcattaatacagttatgccgatcgcagttagaacggttatagcatccaccatttatatctaaaaattagatcaaagaatatgtgacaaagtcctagttgtatactgagaattgacgaaacaatgtttcttacatattttttttttattagtaaccgacttaatagtaggaactggaaaactagacttgattattctataagtatagatacccttccaaataatattctctttgataaaagttccagaaaatgtagaattttttaaaaagttatcttttgctattaccaagattgtgtttagacgcttattattaatatgagtgatgaaatccacaccgcctctagatatcgcttttatttccacattagatggtaaatccaatagtgaaactatctttttaggaatgtatggactcgcgtttagaggagtgaacgtcttaggcgtcggaaaggatgattcatcaaacgaataaacaatttcacaaatggatgttaatgtattagtaggaaattttttgacgctagtggaattgaagattctaatggatgatgttctacctatttcatccgataacatgttaatttccgacaccaacggttttaatatttcgatgatatacggtagtctctctttcggacttatatagcttattccacaatacgagtcattatatactccaaaaaacaaaataactagtataaaatctgtatcgaatgggaaaaacgaaattatcgacataggtatagaatctggaacattgaacgtattaatacttaattctttttctgtggtaagtaccgataggttattgacattgtatggttttaaatattctataacttgagacttgatagatattagtgatgaattgaaaattatttttatcaccacgtgtgtttcaggatcatcgtcgacgcccgtcaaccaaccgaatggagtaaaataaatatcattaatatatgctctagatattagtatttttatcaatcctttgattatcatcttctcgtaggcgaatgattccatgatcaagagtgatttaagaacatcctccggagtattaatgggcttagtaaacagtccatcgttgcaataataaaagttatccaagttaaaggatattatgcattcgtttaaagatatcacctcatctgacggagacaattttttggtaggttttagagactttgaagctacttgtttaacaaagttattcatcgtcgtttactattctatttaattttgtagttaatttatcacatatcacattaattgactttttggtccatttttccatacgtttatattcttttaatcctgcgttatccgtttccgttatatccagggatagatcttgcaagttaaatagaatgctcttaaataatgtcattttcttatccgctaaaaatttaaagaatgtataaacctttttcagagatttgaaactcttaggtggtgtcctagtacacaatatcataaacaaactaataaacattccacattcagattccaacagctgattaacttctacattaatacagcctattttcgctccaaatgtacattcgaaaaatctgaataaaacatcgatgtcacaatttgtattatccaatacagaatgtttgtgattcgtgttaaaaccatcggagaaggaataaaaataaaaattattatagtggtggaattcagttggaatattgcctccggagtcataaaaggatactaaacattgttttttatcataaattacacatttccaatgagacaaataacaaaatccaaacattacaaatctagaggtagaacttttaattttgtctttaagtatatacgataagatatgtttattcataaacgcgtcaaatttttcatgaatcgctaaggagtttaagaatctcatgtcaaattgtcctatataatccacttcggatccataagcaaactgagagactaagttcttaatacttcgattgctcatccaggctcctctctcaggctctattttcatcttgacgacctttggattttcaccagtatgtattcctttacgtgataaatcatcgattttcaaatccatttgtgagaagtctatcgccttagatactttttcccgtagtcgaggtttaaagaaatacgctaacggtatactagtaggtaactcaaagacatcatatatagaatggtaacgcgtctttaactcgtcggttaactctttcttttgatcgagttcgtcgctactattgggtctgctcaggtgccccgactctactagttccaacatcataccgataggaatacaagacactttgccggcggttgtagatttatcatatttctccactacatatccgttacaatttgttaaaaatttagatacatctatattgctacataatccagctagtgaatatatatgacataataaattggtaaatcctagttctggtattttactaattactaaatctgtatatctttccatttatcatggaaaagaatttaccagatatcttcttttttccaaactgcgttaatgtattctcttacaaatattcacaagatgaattcagtaatatgagtaaaacggaacgtgatagtttctcattggcggtgtttccagttataaaacatagatggcataacgcacacgttgtaaaacataaaggaatatacaaagttagtacagaagcacgtggaaaaaaagtatctcctccatcactaggaaaacccgcacacataaacctaaccgcgaagcaatatatatacagtgaacacacaataagctttgaatgttatagttttctaaaatgtataacaaatacagaaatcaattcgttcgatgagtatatattaagaggactattagaagctggtaatagtttacagatattttccaattccgtaggtaaacgaacagatactataggtgtactagggaataagtatccatttagcaaaattccattggcctcattaactcctaaagcacaacgagagatattttcagcgtggatttctcatagacctgtagttttaactggaggaactggagtgggtaagacgtcacaggtacccaagttattgctttggtttaattatttatttggtggattctctactctagataaaatcactgactttcacgaaagaccagtcattctatctcttcctaggatagctttagttagattgcatagcaataccattttaaaatcattgggatttaaggtactagatggatctcctatttctttacggtacggatctataccggaagaattaataaacaaacaaccaaaaaaatatggaattgtattttctacccataagttatctctaacaaaactatttagttatggcactcttattatagacgaagttcatgagcatgatcaaataggagatattattatagcagtagcgagaaagcatcatacgaaaatagattctatgtttttaatgactgccacgttagaggatgacagggaacggctaaaagtatttttacctaatcccgcatttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtagagtttttgtccccgctccttttggaggatcgcaagaatttatttctaaatctatgagagatcaacgaaaaggaagagtaggaagagttaatcctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaatcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttgggtatctctagtgttacacatggccttactaaagtttgggtaaataactatgatatctctattaattatagatgcatatatttcatttgtcaaggatattagtatcgacttgctatcgtcattaatacgtgtaatgtaatcatataaatcatgcgatagccaaggaaaatttaaatagatgttcatcatataatcgtcgctataattcatattaatacgttgacattgactaatttgtaatatagcctcgccacgaagaaagctctcgtattcagtttcatcgataaaggataccgttaaatataactggttgccgatagtctcatagtctattaagtggtaagtttcgtacaaatacagaatccctaaaatattatctaatgttggattaatctttaccataactgtataaaatggagacggagtcataactattttaccgtttgtacttactggaatagacgaaggaataatctccggacatgctggtaaagacccaaatgtctgtttgaagaaatccaatgttccaggtcctaatctcttaacaaaaattacgatattcgatcccgatatcctttgcattctatttaccagcatatcacgaactatattaagattatctatcatgtctattctcccaccgttatataaatcgcctccgctaagaaacgttagtatatccatacaatggaatacttcatttctaaaatagtattcgttttctaattctttaatgtgaaatcgtatactagaaagggaaaaattatctttgagttttccgttagaaaagaaccacgaaactaatgttctgattgcgtccgattccgttgctgaattaatggatttacaccaaaaactcatataacttctagatgtagaagcattcgctaaaaaattagtagaatcaaaggatataagtagatgttccaacaagtgagcaattcccaagatttcatctatatcattctcgaatccgaaattagaaattcccaagtagatatcctttttcatccgatcgttgatgaaaatacgaactttattcggtaagacaatcatttactaaggagtaaaataggaagtaatgttcgtatgtcgttatcatcgtataaattaaaggtgtgttttttaccattaagtgacattataattttaccaatattggaattataatataggtgtatttgcgcactcgcgacggttgatgcatcggtaaatatagctgtatctaatgttctagtcggtatttcatcatttcgctgtctaataatagcgttttctctatctgtttccattacagctgcctgaagtttattggtcggataatatgtaaaataataagaaatacatacgaataacaaaaataaaataagatataataaagatgccatttagagatctaattttgtttaacttgtccaaattcctacttacagaagatgaggaatcgttggagatagtgtcttccttatgtagaggatttgaaatatcttatgatgacttgataacttactttccagataggaaataccataaatatatttctaaagtatttgaacatgtagatttatcggaggaattaagtatggaattccatgatacaactctgagagatttagtctatcttagattgtacaagtattccaagtgtatacggccgtgttataaattaggagataatctaaaaggcatagttgttataaaggacaggaatatttatattagagaagcaaatgatgacttgatagaatatctcctcaaggaatacactcctcagatttatacatattctaatgagcgcgtccccataactggttcaaaattaattctttgtggattttctcaagttacatttatggcgtatacaacgtcgcatataacaacaaataaaaaggtagatgttctcgtttccaaaaaatgtatagatgaactagtcgatccaataaattatcaaatacttcaaaatttatttgataaaggaagcggaacaataaacaaaatactcaggaagatattttattcggtaaccggtggccaaactccataatttgctttttctatttcggattttagaatttccaaattcaccagcgatttatcggttttggtgaaatccaaggatttattaatgtccacaaatgccatttgttttgtctgtggattgtatttgaaaatggaaacgatgtagttagatagatgcgctgcaaagtttcctattagggttccgcgctttacgtcacccagcatacttgaatcaccatcctttaaaaaaaatgataagatatcaacatggagtatatcatactcggattttaattcttctactgcatcactgacattttcacaaatactacaatacggtttaccgaaaataatcagtacgttcttcatttatgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctactgataatcatcctaagatgataaaaaacttaactcaactgtttaaatttgttccctcggcagaggataactatttagcaaaattaacggcgttagtgaatggatgtgatttctttcctggactctatggggcatctataacacccaccaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatgttcctccttgtcaatgcactgttcaagaatttatattttccgcattagatgaaaaatggaacaattttaaatcatcttatttagagaccgttccgttaccctgccaattaatgtatgcattagaaccacgcaaggagattgatgtttcagaagttaaaactttatcatcttatatagatttcgaaaatactaaatcagatatcgatgttataaaatctatatcttcgatcttcggatattctaacgaaaactgtaacactatagtgttcggcatctataaggataatttactactgagtataaatagttcattttactttaacgatagtctgttaataaccaatactaaaagtgataatataataaatataggttactagattaaaaatggtgttccaactcgtgtgctctacgtgcggcaaagatatttctcacgaacgatataaattgattatacgaaaaaaatcattaaaggatgtactcgtcagtgtaaagaacgaatgttgtaggttaaaattatctacacaaatagaacctcaacgtaacttaacagtgcaacctctattggatataaactaatatggatccggttaattttatcaagacatatgcgcctagaggttctattatttttattaattataccatgtcattaacaagtcatttgaatccatcgatagaaaaacatgtgggtatttattatggtacgttattatcggaacacttggtagttgaatctacctatagaaaaggagttcgaatagtcccattggatagtttttttgaaggatatcttagtgcaaaagtatacatgttagagaatattcaagttatgaaaatagcagctgatacgtcattaactttattgggtattccgtatggatttggtcataatagaatgtattgttttaaattggtagctgaatgttataaaaatgccggtattgatacatcgtctaaacgaatattaggtaaagatatttttctgagccaaaacttcacagatgataatagatggataaagatatatgattctaataatttaacattttggcaaattgattaccttaaagggtgagttaatatgcataactactcctccgttgttttttccctcgttctttttcttaacgttgtttgccatcactctcataatgtaaagatattctaaaatggtaaacttttgcatatcggacgcagaaattggtataaatgttgtaattgtattatttcccgtcaatggactagtcacagctccatcagttttatatcctttagagtatttctcactcgtgtctaacattctagagcattccatgatctgtttatcgttgatattggccggaaagatagattttttattttttattatattactattggcaattgtagatataacttctggtaaatatttttctaccttttcaatctcttctattttcaagccggctatatattctgctatattgttgctagtatcaataccttttctggctaagaagtcatatgtggtattcactatatcagttttaactggtagttccattagcctttccacttctgcagaataatcagaaattggttctttaccagaaaatccagctactataataggctcaccgatgatcattggcaaaatcctatattgtaccagattaatgagagcatatttcatttccaataattctgctagttcttgagacattgatttatttgatgaatctagttggttctctagatactctaccatttctgccgcatacaataacttgttagataaaatcagggttatcaaagtgtttagcgtggctagaatagtgggcttgcatgtattaaagaatgcggtagtatgagtaaaccgttttaacgaattatatagtctccagaaatctgtggcgttacatacatgagccgaatgacatcgaagattgtccaatatttttaatagctgctctttgtccattatttctatatttgactcgcaacaattgtagataccattaatcactgattcctttttcgatgccggacaatagcacaattgtttagctttggactctatgtattcagaattaatagatatatctctcaatacagattgcactatacattttgaaactatgtcaaaaattgtagaacgacgctgttctgcagccatttaactttaaataatttacaaaaatttaaaatgagcatccgtataaaaatcgataaactgcgccaaattgtggcatatttttcagagttcagtgaagaagtatctataaatgtagactcgacggatgagttaatgtatatttttgccgccttgggcggatctgtaaacatttgggccattatacctctcagtgcatcagtgttctaccgcggagccgaaaacattgtgtttaatcttcctgtgtccaaggtaaaatcgtgtttgtgtagttttcacaatgatgccatcatagatatagaacctgatctggaaaataatctagtaaaactttctagttatcatgtagtaagtgtcgattgtaacaaggaactgatgcctattaggacagatactactatttgtctaagtatagatcaaaagaaatcttacgtgtttaattttcacaagtatgaagaaaaatgttgtggtagaaccgtcattcatttagaatggttgttgggctttatcaagtgtattagtcagcatcagcatttggctattatgtttaaagatgacaatattattatgaagactcctggtaatactgatgcgttttccagggaatattctatgactgaatgttctcaagaactacaaaagttttctttcaaaatagctatctcgtctctcaacaaactacgaggattcaaaaagagagtcaatgtttttgaaactagaatcgtaatggataatgacgataacattctaggaatgttgttttcggatagagttcaatcctttaagatcaacatctttatgacgtttttagattaatactttcaatgagataaatatgggtggcagagtaagtgttgagctccctaaacgggatccgcctccgggagtacccactgatgagatgttattaaacgtggataaaatgcatgacgtgatagctcccgctaagcttttagaatatgtgcatataggaccactagcaaaagataaagaggataaagtaaagaaaagatatccagagtttagattagtcaacacaggacccggtggtctttcggcattgttaagacaatcgtataatggaaccgcacccaattgctgtcgcacttttaatcgtactcattattggaaaaaggatggaaagatatcagataagtatgaagagggtgcagtattagaatcgtgttggccagacgttcacgacactggaaaatgcgatgttgatttattcgactggtgtcagggggatacgttcgatagaaacatatgccatcagtggatcggttcagcctttaataggagtgatagaactgtagagggtcaacaatcgttaataaatctgtataataagatgcaaacattatgtagtaaagatgctagtgtaccaatatgtgaatcatttttgcatcatttacgcgcacacaatacagaagatagcaaagagatgatcgattatattctaagacaacagtctgcggactttaaacagaaatatatgagatgtagttatcccactagagataagttagaagagtcattaaaatatgcggaacctcgagaatgttgggatccagagtgttcgaatgccaatgttaatttcttactaacacgtaattataataatttaggactttgcaatattgtacgatgtaatactagcgtgaacaacttacagatggataaaacttcctcattaagattgtcatgtggattaagcaatagtgatagattttctactgttcccgtcaatagagcaaaagtagttcaacataatattaaacattcgttcgacctaaaattgcatttgatcagtttattatctctcttggtaatatggatactaattgtagctatttaaatgggtgccgcggcaagcatacagacgacggtgaatacactcagcgaacgtatctcgtctaaattagaacaagaagcgaacgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacgagtgtaaacactgttgttagagattttgaaaattatgtgaaacagacttgtaattctagcgcggtcgtcgataacaaattaaagatacaaaacgtaatcatagatgaatgttacggagccccaggatctccaacaaatttggaatttattaatacaggatctagcaaaggaaattgtgccattaaagcgttgatgcaattgacgactaaggccactactcaaatagcacctagacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgaaaatatattgataatattttaatagattaacatggaagttatcgctgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaattatcaaatgttgttggcgttggtggcgctagtcatcacattaactattttttattactttatactataatagtactagactgacttctaacaaacatctcacctgccataaataaatgcttgatattaaagtcttctatttctaacactattccatctgtggaaaataatactctgacattatcgctaattgacacatcggtgagtgatatgcctataaagtaataatcttctttgggcacatataccagtgtaccaggttctaacaacctatttactggtgctcctgtagcatactttttctttaccttgagaatatccatcgtttgcttggtcaatagcgatatgtgattttttatcaaccactcaaaaaagtaattggagtgttcatatcctctacgggctattgtctcatggccgtgtatgaaatttaagtaacacgactgtggtagatttgttctatagagccggttgccgcaaatagatagaactaccaatatgtctgtacaaatgttaaacattaattgattaacagaaaaaacaatgttcgttctgggaatagaaaccagatcaaaacaaaattcgttagaatatatgccacgtttatacatggaatataaaataactacagtttgaaaaataacagtatcatttaaacatttaacttgcggggttaatttcacaactttactgtttttaaactgttcaaaatatagcatcgatccatgagaaatacgtttagccgcctttaatagaggaaatcccaccgcctttctggatctcaccaacgacgatagttctgaccagcaacttatttcttcatcatccacctgttttaacatataataggcaggagatagatatccgtcattgcaatattccttttcgtaggcacacaatctaatattgataaaatctccattctcttctctgcatttattatcttgtttcggtggctgattaggctgtagtcttggtttaggctttggtatatcgttgttgaatctattttggtcattaaatctttcatttcttcctggtatatttctatcacctcgtttggttggatttttgtctatattatcgtttgtaacatcggtacgggtattcatttatcacaaaaaaaacttctctaaatgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgagaatatcacaggggcaatgtttaccacaatgtataatataagaaacaatttgggtctaggagatgataaactaactattgaagccattgaaaactatttcttggatcctaacaatgaggttatgcctcttatcattaataatacggatatgactgccgtcattcctaaaaaaagtggtaggagaaagaataagaacatggttattttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatgtcctgaatgtgtattctccttccacatccatgagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcattgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagagataataggaggtaatgatatgtatcaatcggtgtgtagaaagtgttacgtcggctcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacgtaaaatatctagcaaaaattctatgtctaaaaacagaaatagcaagagatccgtacgcagtaataaatagaaacgtactactgagatatactaccgatatagagtataatgatttagttactttaataaccgttagacataaaattgattctatgaaaactgtgtttcaggtatttaacgaatcatccataaattatactccggttgatgatgattatggagaaccaatcattataacatcgtatcttcaaaaaggtcataacaagtttcctgtaaattttctatacatagatgtggtaatatctgacttatttcctagctttgttagactagatactacagaaactaatatagttaatagtgtactacaaacaggcgatggtaaaaagactcttcgtcttcccaaaatgttagagacggaaatagttgtcaagattctctaccgtcctaatataccattaaaaattgttagatttttccgcaataacatggtaactggagtagagatagccgatagatctgttatttcagtcgctgattaatcaattagtagagatgagataagaacattataataatcaataatatattttatatcttatatcttgtttagaaaaatgctaatattaaaatagctaacgctagtaatccaatcggaagccatttgatatctataatagggtatctaatttcctgattcagatagcggacagctatattctcggtagctactcgtttggaatcacaaacattatttacatctaatttactatctgtaatggaaacgtttcccaatgaaatggtacaatccgatacattgcattttgttatatttttttttaaagaggctggtaacaacgcatcgcttcgtttacatggctcgtaccaacaataatagggtaatcttgtatctattcctatccgtactatgcttttatcaggataaatacatttacatcgtatatcgtctttgttagcatcacagaatgcataaatttgttcgtccgtcatgataaaaatttaaagtgtaaatataactattattttatagttgtaataaaaagggaaatttgattgtatactttcggttctttaaaagaaactgacttgataaaaatggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaaaacttgtgggaaaacggaattggaatgtttcggtcactggggtaaagtaagtatttataaaactcatatagttaagcctgaatttatttcagaaattattcgtttactgaattatatatgtattcactgcggattattgcgttcacgagaaccgtattccgacgatattaacctaaaagagttatcgggacacgctcttaggagattaaaggataaaatattatccaagaaaaagtcatgttggaacagtgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgttgattattagaccggctattagtttttggatagatagtatacccaaagaaaccaatgaattaacttacttattaggtatgatcgttaagaattgtaacttgaatgctgatgaacaggttatccagaaggcggtaatagaatacgatgatattaaaattatttctaataacacttccagtatcaatttatcatatatcacatccggcaaaaataatatgattagaagttatatcgtcgcccggcgaaaagatcagaccgctagatctgtaattggtcccagtacatctatcaccgttaatgaggtaggaatgcccgcatatattagaaatacacttacagaaaagatatttgttaatgcctttacagtggataaagttaaacaactattagcgtcaaaccaagttaaattttactttaataaacgattaaaccaattaacaagaatacgccaaggaaagtttatcaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgtcaactctcaaaatgctgatttcgacggagatgaagaatggatgatattggagcaaaatcctaaagccgtaattgaacaaagtattcttatgtatccgacgacgttactcaaacacgatattcatggagcccccgtttatggatctattcaagatgaaatcgtagcagcgtattcattgtttaggatacaagatctttgtttagatgaagtattgaacatcttggggaaatatggaagagagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcccacagaactaatgtatattctaggtacttatggacaacagaggattgatggtgaaccagcagagactcgagtattgggtagagtcttaccttactatcttccagactctaaggatccagaaggaagaggttacattcttaattctttaacaaaaggattaacgggttctcaatattacttttcgatgctggttgcaagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaaaaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacttgactaatttgagtaagaataagaccgaaattatcactctggtatccgatgatatctctaaacttcaatctgttaagattaatttcgaatttgtatgtttgggagaattaaatccaaacatcactcttcgaaaagaaacagataggtatgtagtagatataatagtcaatagattatacatcaagagagcagaaattaccgaattagtcgtcgaatatatgattgaacgattcatctcctttagcgtcattgtaaaggaatggggtatggaaacattcattgaggatgaggataatattagatttactgtctacctaaatttcgttgaaccggaagaattgaatcttagtaagtttatgatggttcttccgggtgccgccaacaagggcaagattagtaaattcaagattcctatctctgattatacgggatatgacgacttcaatcaaacaaaaaagctcaataagatgactgtagaactcatgaatctaaaagaattgggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaattcttatcaataacatatttttctatgatctgtcttttaaacgatggattttccacaaatgcgcctctcaagtccctcatagaatgatacacgtataaaaaatatagcataggcaatgactccttatttttagacattagatatgccaaaatcatagccccgcttctatttactcccgcagcacaatgaaccaacacgggctcgtttcgttgatcacatttagataaaaaggcggttacgtcgtcaaaatatttactaatatcggtagttgtatcatctaccaacggtatatgaataatattaatattagagttaggtaatgtatatttatccatcgtcaaatttaaaacatatttgaacttaacttcagatgatggtgcatccatagcatttttataatttcccaaatacacattattggttactcttgtcattatagtgggagatttggctttgtgcatatctccagttgaacgtagtagtaagtatttatacaaacttttcttatccatttataacgtacaaatggataaaactactttatcggtaaacgcgtgtaatttagaatacgttagagaaaaggctatagtaggcgtacaagcagccaaaacatcaacacttatattctttgttattatattggcaattagtgcgctattactctggtttcagacgtctgataatccagtctttaatgaattaacgagatatatgcgaattaaaaatacggttaacgattggaaatcattaacggatagcaaaacaaaattagaaagtgatagaggtagacttctagccgctggtaaggatgatatattcgaattcaaatgtgtggatttcggcgcctattttatagctatgcgattggataagaaaacatatctgccgcaagctattaggcgaggtactggagacgcgtggatggttaaaaaggcggcaaaggtcgatccatctgctcaacaattttgtcagtatttgataaaacacaagtctaataatgttattacttgtggtaatgagatgttaaatgaattaggttatagcggttattttatgtcaccgcattggtgttccgattttagtaatatggaatagtgttagataaatgcggtaacgaatgttcctgtaaggaaccataacagcttagatttaacgttaaagatgagcataaacataataaacaaaattacaatcaaacctataacattaatatcaaacaatccaaaaaatgaaatcagtggagtagtaaacgcgtacataactcctggataacgtttagcagctgccgttcctattctagaccaaaaattcggtttcatgttttcgaaacggtattctgcaacaagtcgaggatcgtgttctacatatttggcggcgttatccagtatctgcctattgatcttcatttcgttttcgattctggctatttcaaaataaaatcccgatgatagacctccagactttataatttcatctacgatgttcagcgccgtagtaactctaataatataggctgataagctaacatcataccctcctgtatatgtgaatatggcatgatttttgtccattacaagctcggttttaactttattgcctgtaataatttctctcatctgtaggatatctatttttttgtcatgcattgccttcaagacgggacgaagaaacgtaatatcctcaataacgttatcgttttctacaataactacatattctacctttttattttctaactcggtaaaaaaattagaatcccatagggctaaatgtctagcgatatttcttttcgtttcctctgtacacatagtgttacaaaaccctgaaaagaagtgagtatacttgtcatcatttctaatgtttcctccagtccactgtataaacgcataatccttgtaatgatctggatcatccttgactaccacaacatttcttttttctggcataacttcattgtcctttacatcatcgaacttctgatcattaatatgctcatgaacattaggaaatgtttctgatggaggtctatcaataactggcacaacaataacaggagttttcaccgccgccatttagttattgaaattaatcatatacaactctttaatacgagttatattttcgtctatccattgtttcacatttacatatttcgacaaaaagatataaaatgcgtattccaatgcttctctgtttaatgaattactaaaatatacaaacacgtcactgtctggcaataaatgatatcttagaatattgtaacaatttattttgtattgcacatgttcgtgatctatgagttcttcttcgaatggcataggatctccgaatctgaaaacgtataaataggagttagaataataatatttgagagtattggtaatatataaactctttagcggtataattagtttttttctctcgatttctatttttagatgtgatggaaaaatgactaattttgtagcattagtatcatgaactctaatcgagatcttaatatcttcgtcacacgttagttctttgaagtttttaagagatgcatcagttggttcgaccgatggagtaggtgcaacaattttttgttcgatgtatgtatgtactggagccattgtcttaactataatggtgcttgtatcgaaaaactttaatgcagataatggaagctcttcgccgcgactttctacatcgtaattgggttctaacgccgatctctgaatggatactagttttctaagttctaatgtgattctctgaaaatgtaaatccaattcctccggcattatagatgtgtatacatcggtaaataaaactatagtatccaacgatcccttctcgcaaattctagtcttaaccaaaaaatcgtatataaccacggagatggcgtatttaagagtggattcttctaccgttttgttcttggatttcatataagaaactataaagtccgcactactgttaagaatgattactaacgcaactatatagtttaaattaagcatcttggaaacataaaataactctgtagacgatacttgactttcgaataagtttgcagacaaacgaagaaagaacagacctctcttaatttcagaagaaaactttttttcgtattcctgacgtctagagtttatatcaataagaaagttaagaattagtcggttaatgttgtatttcattacccaagtttgagatttcataatattatcaaaagacatgataatattaaagataaagcgctgactatgaacgaaatagctatatggttcgctcaagaatatagtcttgttaaacgtggaaacgataactgtatttttaatcacgtcagcggcatctaaattaaatataggtatatttattccacacactctacaatatgccacaccatcttcataataaataaattcgttagcaaaattattaattttagtgaaatagttagcgtcaactttcatagcttccttcaatctaatttgatgctcacacggtgcgaattccactctaacatcccttttccatgcctcaggttcatcgatctctataatatctagttttttgcgtttcacaaacacaggctcgtctctcgcgatgagatctgtatagtaactatgtaaatgataactagatagaaagatgtagctatatagatgacgatcctttaagagaggtatgatgactttaccccaatcagatagactgttgttatggtcttcggaaaaagaatttttataaatttttccagtattttccaaatatacgtacttaacatctaaaaaatccttaatgataataggaatggataatccgtctattttataaagaaatacatatcgcacattatacttttttttggaaatgggaataccgatgtgtctacataaatatgcaaagtctaaatattttttagagaatcttagttggtccaaattcttttccaagtacggtaatagatttttcatattgaacggtatcttcttaatctctggttctagttccgcattaaatgatgaaactaagtcactatttttataactaacgattacatcacctctaacatcatcatttaccagaatactgatcttcttttgtcgtaaatacatgtctaatgtgttaaaaaaaagatcatacaagttatacgtcatttcatctgtggtattcttgtcattgaaggataaactcgtactaatctcttctttaacagcctgttcaaatttatatcctatatacgaaaaaatagcaaccagtgtttgatcatccgcgtcaatattctgttctatcgtagtgtataacaatcgtatatcttcttctgtgatagtcgatacgttataaaggttgataacgaaaatatttttatttcgtgagataaagtcatcgtaggattttggacttatattcgcgtctagtagatatgcttttatttttggaatgatctcaattagaatagtctctttagagtccatttaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatggcgtggtcaattacaaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggctgctgtagagctcgatgatagcgacggggatgatgaacctatggtacaagttgaagctggtaaagtaaatcatagtgctagaagcgatctttctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaaatgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataaaccgctattgaaactgacggatgattctagtcccgaagaatttctgttcaacaaactaagtgaacgaaaggtatacgaatgtatcaaacagtttggtattagaatcaaggatctccgaacgtatggagtcaattatacgtttttatataatttttggacaaatgtaaagtccatatctcctcttccgtcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatgatatagatctttacacaaataattacaagaccgataaatggaaatggataagcgtatgaaatctctcgcaatgacagctttcttcggagagctaaacacattagatattatggcattgataatgtctatatttaaacgccatccaaacaataccattttttcagtggataaggatggtcagtttatgattgatttcgaatacgataattataaggcttctcaatatttggatctgaccctcactccgatatctggagatgaatgcaagactcacgcatcgagtatagccgaacaattggcgtgtgtggatattattaaagaggatattagcgaatatatcaaaactactccccgtcttaaacgatttataaaaaaataccgcaatagatcagatactcgtatcagtcgagatacagaaaagcttaaaatagctctagctaaaggcatagattacgaatatataaaagacgcttgttaataagtaaatgaaaaaaaactagtcgtttataataaaacacaatatggatgccaacatagtatcatcttctactattgcaacgtatatagacgctttagcgaagaatgcttcagaattagaacagaggtctaccgcatacgaaataaataatgaattggaactagtatttattaagccgccattaattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccagtctaaaagttctttattacacgctattaatcatccaaagtcaaggcctaatacatctctggaaatagaattcacacctagagacaatgaaaaagttccatatgatgaactaataaaggaattgacgactctatcacgtcatatatttatggcttctccagagaatgtaattctttctccgcctattaacgcgcctataaaaacctttatgttgcctaaacaagatatagtaggtttggatctggaaaatctatatgccgtaactaagactgacggaattcctataactatcagagttacatcaaaagggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagaatcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagtcgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgcggacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccgtctacaaaaaactttttcgaactaaatagaggagccattaaatgtgaaggtttagatgtcgaagacttacttagttactatgttgtttatgtcttttctaagcggtaaataataatatggtatgggttctgatatccccgttctaaatgcattaaataattccaatagagcgatttttgttcctataggaccttccaactgtggatactctgtattgttaatagatatattaatacttttgtcgggtaacagaggttctacgtcttctaaaaataaaagtttgataacatctggcctgttcataaataaaaacttggcgattctatatatactcttattatcaaatctagccattgtcttatagatgtgagctactgtaggtgtaccatttgattttctttctaatactatatatttctctcgaagaagttcttgcacatcatctgggaataaaatactactgttgagtaaatcagttattttttttatatcgatattgatggacatttttatagttaaggataataagtatcccaaagtcgataacgacgataacgaagtatttatacttttaggaaatcacaatgactttatcagatcaaaattaacaaaattaaaggagcatgtatttttttctgaatatattgtgactccagatacatatggatctttatgcgtcgaattaaatgggtctagttttcagcacggtggtagatatatagaggtggaggaatttatagatgctggaagacaagttagatggtgttctacatccaatcatatatctgaagatatgcacactgataaatttgtcatttatgatatttatacgtttgattcgttcaagaataaacgattggtatttgtacaggtgcctccatcattaggagatgatagctatttgactaatccgttattgtctccgtattatcgtaattcagtagccagacaaatggtcaatgatatgatttttaatcaagattcatttttaaaatatttattagaacatctgattagaagccactatagagtttctaaacatataacaatagttagatacaaggataccgaagaattaaatctaacgagaatatgttataatagagataagtttaaggcgtttgtattcgcttggtttaacggcgtttcggaaaatgaaaaggtactagatacgtataaaaaggtatctaatttgatataatgaattcagtgactgtatcacacgcgccatatactattacttatcacgatgattgggaaccagtaatgagtcaattggtagagttttataacgaagtagccagttggctgctacgagacgagacgtcgcctattcctgataagttctttatacagttgaaacaaccgcttagaaataaacgagtatgtgtgtgtggtatagatccgtatccgaaagatggaactggtgtaccgttcgaatcaccaaattttacaaaaaaatcaattaaggagatagcttcatctatatctagattaaccggagtaattgattataaaggttataaccttaatataatagacggggttataccctggaattattacttaagttgtaaattaggagaaacaaaaagtcacgcgatctactgggataagatttccaagttactgctgcagcatataactaaacacgttagtgttctttattgtttgggtaaaacagatttctcgaatatacgggcaaagttagaatccccggtaactaccatagtcggatatcatccagcggctagagaccgccaattcgagaaagatagatcatttgaaattatcaacgttttactggaattagacaacaaggcacctataaattgggctcaagggtttatttattaatgctttagtgaaattttaacttgtgttctaaatggatgcaactattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggtgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaaccagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacgacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagaccatatagtttggattaataattcatggaaatttaacagcgaagaacccttgataacaaaactaattctgtcaataagacatcaactacctaaggaatattcaagcgaattactctgtccgaggaaacgaaagactgtagaagctaacatacgagacatgttaatagattcagtggagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagttgtacgaaaaaactttatctagttgtttatgcggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtctttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacaaaaaatatcatgttcctattatgaaactatatcctacaccggaagagattccggactttgcattctatctcaaaataggtactctgttagtatctagctctgtaaagcatattccattaatgacggacctctccaaaaagggatatatattgtacgataatgtggttactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaatatatgctcatatatttatagaagatatcacatatctaaatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatattaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttttactattgtctggatctcccattactaacacacctaatactctgggtcatattatagatttaatgtccgaagagacgatagattttggtgaaattattagtcgtggtaagaaagtaattcagacacttcttaacgaacgaggtgtgaatgtacttaaggatttgcttaaaggaagaatatcatattacgaaatgcctgataaagatctaccaacgataagatatcacggacgtaagtttctagatactagagtagtatattgtcacatgtctaaacttcaagagagagattatatgattactagacgacagctatgttatcatgaaatgtttgataaaaatatgtataacgtgtcaatggcagtattgggacaacttaatctgatgaataatttagatactttatttcaggaacaggataaggaattgtacccaaatctgaaaataaataatggcgtgttatacggagaagaattggtaacgttaaacattagttccaaatttaaatactttattaatcggatacagacactcaacggaaaacattttatatacttttctaattctacatatggcggattggtaattaaatatatcatgctcagtaatggatattctgaatataatggttctcagggaactaatccacatatgataaacggcaaaccaaaaacatttgctatcgttactagtaaaatgaaatcgtctttagaggatctattagatgtgtataattctcctgaaaacgatgatggcagtcaattgatgtttttgttttcgtcaaacattatgtccgaatcctatactctgaaagaggtaaggcatatttggtttatgactatcccagatactttttctcaatacaaccaaattcttggacgatctattagaaaattctcttacgccgatatttctgaaccagttaatgtatatcttttagccgccgtatattccgatttcaatgacgaagtgacgtcattaaacgattacacacaggatgaattaattaatgttttaccatttgacatcaaaaagctgttgtatctaaaatttaagactaaagaaacgaatagaatatactctattcttcaagagatgtctgaaacgtattctcttccaccacatccatcaattgtaaaagttttattgggagaattggtcagacaatttttttataataattctcgtattaagtataacgataccaagttacttaaaatggttacatcagttataaaaaataaagaagacgctaggaattacatagatgatattgtaaacggtcacttctttgtatcgaataaagtatttgataaatctcttttatacaaatacgaaaacgatattattacagtaccgtttagactttcctacgaaccatttgtttggggagttaactttcgtaaagaatataacgtggtatcttctccataaaactgatgagatatataaagaaataaatgtcgagctttgttaccaatggataccttccagttacattggagccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgttgactcataaaaaagagaatagcggtaagtataaacacgaatactatggcaataattgcgaatgttttattcccttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatgcggttttccatcatgattagacgacgacaatagtgttctaaatttagatagttgatcagaatgaatgtttattggcgttggaaaaattatccatacagcgtctgcagagtggttgatagttgttcctagatatgtaaaataatccaacttactaggcagcaaattgtctagataaaatactgaatcaaacggtgcagacgtattggtggatctaatggaatccaattgattaactatcttttgaaaatatacatttttatgatccgatacttgtaagaatatagaaataatgataagtccatcatcgtgtttttttgcctcttcataagaactatattttttcttattccaatgaacaagattaatctctccagagtatttgtacacatctatcaagtgattggatccataatcgtcttcctttccccaatatatacgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcatagtaaaaatgaaatgataactgtttaaaaatagctcttagtatgggaattacaatggatgaggaagtgatatttgaaactcctagagaattaatatctattaaacgaataaaagatattccaagatcaaaagacacgcatgtgtttgctgcgtgtataacaagtgacggatatccgttaataggagctagaagaacttcattcgcattccaagcgatattatctcaacaaaattcagattctatctttagagtatccactaaactattacggtttatgtactacaatgaactaagagaaatctttagacggttgagaaaaggttctatcaacgatatcgatcctcactttgaagagttaatattattgggtggtaaactagataaaaaggaatctattaaagattgtttaagaagagaattaaaagaggaaagtgatgaacgtataacagtaaaagaatttggaaatgtaattctaaaacttacaacacgggataaattatttaataaagtatatataagttattgcatggcgtgttttattaatcaatcgttggaggatttatcgcatactagtatttacaatgtagaaattagaaagattaaatcattaaatgattgtattaacgacgataaatacgaatatctgtcttatatttataatatgctagttaatagtaaatgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtctagaaagaaacgattatttctgaattattccaattatttgtccaaacaggaaagaagtatactatcgtcatttttttctctagatccagctactactgataatgatagaatagatgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctttaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattgattagaaaatataaattaatttaccatcgtgtatttttataacgggattgtccggcatatcatgtagatagttaccgtctacatcgtatactcgaccatctacgcctttaaatcctctatttattgacattaatctattagaattggaataccaaatattagtaccctcaattagtttattggtaatattttttttagacgatagatcgatggctcttgaaaccaaggttttccaaccggactcattgtcgatcggtgagaagtctttttcattagcatgaatccattctaatgatgtatgtttaaacactctaaacaattggacaaattcttttgatttgctttgaatgatttcaaataggtcttcgtctacagtaggcataccattagataatctagccattataaagtgcacgtttacatatctacgttctggaggagtaagaacgtgactattgagacgaatggctcttcctactatctgacgaagagacgcctcgttccatgtcatatctaaaatgaagatatcattaattgagaaaaaactaataccctcgcctccactagaagagaatacgcatgttttaatgcattctccgttagtgtttgattcttggttaaactcagccaccgccttgattctagtatcttttgttctagatgagaactctatattagagataccaaagactttgaaatatagtaataagatttctattcctgactgattaacaaatggttcaaagactagacatttaccatgggatgctaatattcccaaacatacatctataaatttgacgcttttctcttttaattcagtaaatagagagatatcagccgcactagcatcccctttcaatagttctccctttttaaaggtatctaatgcggatttagaaaactctctatctcttaatgaatttttaaaatcattatatagtgttgctatctcttgcgcgtattcgcccggatcacgattttgtctttcaggaaagctatcgaacgtaaacgtagtagccatacgtctcagaattctaaatgatgatatacctgtttttatttcagcgagtttagccttttgataaatttcttcttgctttttcgacatattaacgtatcgcattaatactgttttcttagcgaatgatgcagacccttctacgtcatcaaaaatagaaaactcgttattaactatgtacgaacataggcctcctagtttggagactaattctttctcatcaactagacgtttattctcaaatagcgattggtgttgtaaggatcctggtcgtagtaagttaaccaacatggtgaattcttgcacactattaacgataggtgtagccgataaacaaatcatcttatggttttttaatgcgatggtcttagataaaaaattatatactgaacgagtaggacggatcttaccatcttctttgattaatgatttagaaatgaagttatgacattcatcaataatgacgcatattctactcttggaattaatagttttgatattagtaaaaaatttatttctaaaattttgatcatcgtaattaataaaaatacaatccttcgttatctctggagcgtatctgagtatagtgttcatccaaggatcttctatcaaagcctttttcaccaataagataatagcccaattcgtataaatatccttaagatgtttgagaatatatacagtagtcattgttttaccaacacccgtttcatggaacaataaaagagaatgcatactgtctaatcctaagaaaactcttgctacaaaatgttgataatccttgaggcgtactacgtctgttcccatcatttcaacaggcatattagtagttctgcgcaatgcataatcgatataggccgcgtgtgatttactcatttatgagtgataagtaataactatgttttaaaaatcacagcagtagtttaactagtcttctctgatgtttgttttcgatactttttgaatcagaagtcatactagaataaagcaacgagtgaacgtaatagagagcttcgtatactctattcgaaaactctaagaacttattaatgaattccgtatccactggattgtttaaaatactaaattgaacactgttcacatccttccaagaagaagacttagtgacggacttaacatgagacataaataaatccaaattttttttacaaacatcactagccaccataatggcgctatctttcaaccagctatcgcttacgcattttagcagtctaacatttttaaagagactacaatatattctcatagtatcgattacacctctaccgaataaagttggaagtttaataatacaatatttttcgtttacaaaatcaaataatggtcgaaacacgtcgaaggttaacatcttataatcgctaatgtatagattgttttcagtgagatgattattagatttaatagcatctcgttcacgtttgaacagtttattgtgtgcgctgaggtcggcaactacggcgtccgctttagtactcctcccataatactttacgctattaatctttaaaatttcatagactttatctagatcgctttctggtaacatgatatcatgtgtaaaaagttttaacatgtcggtcggcattctatttagatcattaactctagaaatctgaagaaagtaattagctccgtattccagactaggtaatgggcttttacctaaagacaagttaagttctggcaatgtttcataaaatggaagaaggacatgcgttccctcccggatattttttacaatttcatccatttacaactctatagtttgttttcattattattagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattggccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctcaacaatctcggcagactccggataaccagtcggtgggccatcactaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaacgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatacaaagttggaatttgactatcgactgcgaagacattagaccgtttaatagagtcatccccaccgatcaaagaattaatgatagtattattcattttctatttaaaatggaaaaagcttacaataaactccgtagagaaatatctataatttgtgagttttccttaaagtaacagcttccgtaaacgccgtctttatctcttagtaggtttattgtatttatgaccttttccttatcttcatagaatactaaaggcaacaaagaaatttttggttcttctctaagagctacgtgagacttaaccatagaagccaacgaatccctacatattttagaacagaaatacccaacttcaccacccttgaatgtctcaatactaataggtctaaaaaccaaatcttgattacaaaaccaacacttatcaattacactatttgtcttaatagacacatctgccatagatttataatactttggtagtatacaagcgagtgcttcttctttagcgggcttaaagactgctttaggtgctgaaataaccacatctggaaggcttactcgcttagccatttaattacggaactatttttttatacttctaatgagcaagtagaaaacctctcatctacaaaaacatactcgtgtccataatcctctaccatagttacacgttttttagatctcatatgtgctaaaaagttttcccatactaattggttactattatttttcgtataatttttaacagtttgaggttttagatttttagttacagaagtgatatcgaatattttatccaaaaagaatgaataattaattgtcttagaaggagtgttttcttggcaaaagaataccaagtgcttaaatatttctactacttcattaatcttttctgtactcagattcagtttctcatcttttacttgattgattatttcaaagactaacttataatcctttttatttattctctcgttagccttaagaaaactagatacaaaatttgcatctacatcatccgtggatatttgatttttttccatgatatccaagagttccgagataatttctccagaacattgatgagacaataatctccgcaatacatttctcaaatgaataagtttattagacacatggaagtttgactttttttgtacctttgtacatttttgaaatacagactcgcaaaaaatacaatattcatatccttgttcagatactataccgttgtgtctacaaccgctacataatcgtagattcatgttaacactctacgtatctcgtcgtccaatattttatataaaaacattttatttctagacgttgccagaaaatcctgtaatatttttagttttttgggctgtgaataaagtatcgccctaatatggttaccgtcctccgccaatatagtagttaaattatccgcacatgcagaagaacaccgcttaggcggattcagtacaatgttatatttttcgtaccaactcatttaaatatcataatctaaaatagttctgtaatatgtctagcgctaatatattgatcataatcctgtgcataaattaagatacaacaatgtctcgaaatcatcgacatggcttcttccatagttagaagatcgtcgtcaaagttagcaacgtgattcatcaacatttgctgttttgaggcagcaaatactgaaccgtcgccattcaaccattcataaaaaccatcgtctgaatccattgataatttcttgtactggtttttgagagctcgcatcaatctagcatttctagctcccggattgaaaacagaaagaggatcgtacatccagggtccattttctgtaaatagaatcgtataatgtcccttcaagaagatatcagacgatccacaatcaaagaattggtctccgagtttgtaacaaactgcggactttaacctatacatgataccgtttagcatgatttctggtgatacgtcaatcggagtatcatctattagagatctaaagccggtgtaacattctccaccaaacatattcttattctgacgtcgttctacataaaacatcattgctccattaacgataacaggggaatgaacagcactacccatcacattagttcccaatggatcaatgtgtgtaactccagaacatcttccatatcctatgttaggaggagcgaacaccactcttccactattgccatcgaatgccatagaataaatatccttggaattgatagaaatcggactgtcggatgttgtgatcatcttcataggattaacaactatgtatggtgccgcctgaagtttcatatcgtaactgatgccgtttataggtctagccacagaaaccaacgtaggtctaaatccaactatagacaaaatagaagccaatatctgttcttcatctgtcataacttgagagcatccagtatgaataatcttcattagatggggatctaccgcatcatcatcgttacaataaaaaattcccattctaatgttcataattgcttttctaatcatggtatgcatgtttgctctctgaatctctgtggaaattagatctgatacacctgtaatcactatcggattatcctccgtaagacgattaaccaacaacatataattataagactttacttttctaaattcataaagttgctggattaggctataggtgtctccatgtacatacgcgttctcgagcgcaggaagtttaataccgaatagtgccatcagaataggatgaatatagtaattagtttctggttttctataaataaaagacaaatcttgtgaactagacatatcggtaaaatgcatggattggaatcgtgtagtcgacagaagaatatgatgattagatggagagtatattttatctaactctttgagttggtcaccgattctaggactagctcgagaatgaataagtactaaaggatgagtacatttcacagaaacactagcattgttcaatgtgctctttacatgggtaaggagttgaaatagctcgtttctatttgttctgacaatatttagtttattcataatgttaagcatatcctgaatagtaaagttagatgtgtcatacttgttagtagttagatatttagcaattgcattcccatcatttctcaatctcgtactccaatcatgcgtggatgctacttcgtcgatggaaaccatacaatcctttttgataggctgttgagattgattatttcctgcacgtttaggtttggtacgttgatttctagcccctgcggatataaagtcatcgtctacaattttggataatgaattgcatacactacaagacaaagatttatcagaagtgtgaatatgatcttcatctaccaaagaaagagtttgattagtataactagattttagtcctgcgttagatgttaaaaaaacatcgctattgaccacggcttccattatttatattcgtagtttttactcgaaagcgtgattttaatattcaatcttattacttttggaatcgttcaaaacctttgactaattgtagaatttgatctattgccctacgcgtatactcccttgcatcatatacgttcgtcaccagatcgtttgtttcggcctgaagttggtgcatatctctttcaacattcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatacttccatccgattgttgtgtacatgtagttgttggtaacgtctgagaaggttgggtagatggcggcgtcgtcgtcttttgatctttattaaatttagagataatatcctgaacagcattgctcggcgtcaacgctggaaggagtgaactcgccggcgcatcagtatctgcagacagccaatcaaaaagattagacatatcagatgatgtattagtttgttgtcgtggttttggtgtaggagccggtgtagctgttggaaccggctgtggagttatatgaatagttggttgtagcggttggataggctgtctgctggcggccatcatattatctctagctagttgttctcgcaactgtctttgataatacgactcttgagactttagtcctatttcaatcgcttcatcctttttcgtatccggatccttttcttcagaataatagattgacgactttggtgtagaggattctgccagcccctgtgagaacttgttaaagaagtccatttaaggctttaaaattgaattgcgattataagattaaatggcagacacagacgatattatcgactatgaatccgatgatctcaccgaatacgaggatgatgaagaagaggaagaagatggagagtcactagaaactagtgatatagatcccaaatcttcttataagattgtagaatcagcatccactcatatagaagatgcgcattccaatcttaaacatatagggaatcatatatctgctcttaaacgacgctatactagacgtataagtctatttgaaatagcgggtataatagcagaaagctataacttgcttcaacgaggaagattacctctagtttcagaattttctgacgaaacgatgaagcaaaatatgctacatgtaattatacaagagatagaggagggttcttgtcctatagtcatcgaaaagaacggagaattgttgtcggtaaacgattttgacaaagatggtctaaaattccatctagactatattatcaaaatttggaaacttcaaaaacgatattagaatttatacgaatatcgttctctaaatgtcacaatcaagtctcgcatgttcagcaatttattgtcgtactttatatcgtgttcattaacgatatcttgcaaaatagtaatgattctatcttccttcgatagatattcttcagagattattgtcttatattctttcttgttatcagatatgaatttgataagactttgaacattattgatacccgtctgtttaattttttctacagatattttagttttggcagattctatcgtatctgtcaatagacatccaacatcgacattcgacgtcaattgtctataaatcaacgtataaattttagaaataacattagcgaattgttgtgcattgatgtcgttattctgaaacagtatgattttaggtagcattttcttaacaaagagaacgtatttattgttactcagttgaacagatgatatatccagattactaacgcatctgattccatataccaaactttcagaagaaatggtgtacaattgtttgtattcattcaatgtctctttttcagaaattagtttagagtcgaatactgcaataattttcaagagatagttttcatcagataagattttatttagtgtagatatgataaaactattgttttgttggagaacttgatacgccgcgttctctgtagtcgacgctctcaaatgggaaacaatctccattatttttttggaatcggatacaatatcttcggtatcttgacgcaatctagtatacatagagttaagagaaattagagtttgtacattaagcaacatgtctctaaatgtggctgcaaacttttccttttccacatcatctagtttattatataccgatttcacaacggcaccagatttaaggaaccagaatgaaaaactctgataactacaatatttcatcatagttacgattttatcatcttctatagttggtgtaatagcgcatacctttttctccaagactggaaccaacgtcataaaaatgtttaaatcaaaatccatatcaacatctgatgcgctaagaccagtctcgcgttcaagattatctttactaatggtgacgaactcatcgtatagaactctaagtttgtccattatttatttacagatttagttgtttaatttatttgtgctcttccagagttgggatagtatttttctaacgtcggtattatattattaggatctacgttcatatgtatcataatattaatcatccacgttttgataaatctatctttagcttctgaaataacgtatttaaacaaaggagaaaaatatttagctacggcatcagacgcaataacattttttgtaaatgtaacgtatttagacgacagatcttcgttaaaaagttttccatctatgtagaatccatcggttgttaacaccattcccgcgtcagattgaataggagtttgaatagtttgttttggaaatagatccttcaataacttatagttgggtgggaaaaaatcgattttatcactagactctttcttttttactatcattacctcatgaactatttcttgaatgagtatatgtattttctttcctatatcggacgcgttcattggaaaatataccatgtcgttaactataagaatatttttatcctcgtttacaaactgaataatatcagatgtagttcgtaaacgaactatatcatcaccagcacaacatctaactatatgatatccactagtttcctttagtcgtttattatcttgttccatattagcagtcattccatcatttaagaaggcgtcaaagataatagggagaaatgacattttggattctgttacaactttaccaaaattaaggatatacggacttactatctttttctcaacgtcgatttgatgaacacacgatgaaaatgtacttcgatgagattgatcatgtagaaaacaacaagggatacaatatttccacatatcatgaaatatattaagaaatcccaccttattatatttccccaaaggatccatgcatgtaaacattatgccgttatcattaataaagacttctttctcatcggatctgtaaaagttgttactgatttttttcattccaggatctagataattaataatgatgggttttctattcttattctttgtattttggcatatcctagaccagtaaacagtttccactttggtaaaatcagcagacttttgaacgctattaaacatggcattaatggcaataactaaaaatgtaaaatatttttctatgttaggaatatggtttttcactttaatagatatatggtttttggccaaaatgatagatatttttttatccgaggatagtaaaatattattagtcgccgtctctataaaaatgaagctagtctcgatatccaattttattctagaattgataggagtcgccaaatgtaccttatacgttatatctcccttgatgcgttccatttgtgtatctatatcggacacaagatctgtaaatagttttacgttattaatcatcacggtatcgccgtcgctagataacgctaatgtaccatccaagtcccaaatggagagatttaactgttcatcgtttagaataaaatgattaccggtcatattaataaagtgttcatcgtatctagataacaacgacttataattaatgtccaagtcttgaactcgctgaatgatcttttttaacccagttagttttagattggtacgaaatatattgttaaactttgattctacagtaatgtccaaatctagttgtggaaatacttccatcaacattgtttcaaacttgataatattattatctacatcttcatacgatccaaattccggaatagatgtatcgcacgctctggccacccagataaccaaaaagtcacacgctccaggatatacattgtataaaaagctatcgttttttagtagtgtttttttctgagtatatacgaagggattaaaaatagtattatcaacgtaactatattccaaattattcttatgagaatagataataatatcgtccttaatatctaacaaatttcctaaatatccctttaattgagtcattcgaagcgtcaatagaatatgtctcttaactatttccggctgttgtatatttaaatgacttcgtaaaaaataatatatgggcgacttctcatctatgtaatcatatggagtgagatatagggctcgttctacctcctgccccttacccacctgtaataccaattgcggacttactatatatcgcatatttatatcgtggggtaaagtgaaaatctactaccgatgatgtaagtcttacaatgttcgaaccagtaccagatcttaatttggaggcctccgtagaactaggggaggtaaatatagatcaaacaacacctatgataaaggaaaatagcggttttatatcccgtagtagacgtctattcgcccatagatctaaggatgatgagagaaaactagcactacgattctttttacaaagactttattttttagatcatagagagattcattatttgttcagatgcgttgacgctgtaaaagacgtcactattaccaaaaaaaataacattatcgtggcgccttatatagcacttttaactatcgcatcaaaaggatgcaaacttacagaaacaatgattgaagcattctttccagaactatataatgaacatagtaagaaatttaaattcaactctcaagtatccatcatccaagaaaaactcggataccagtttggaaactatcacgtttatgattttgaaccgtattactctacagtagctctggctattcgagatgaacattcatctggcatttttaatatccgtcaagagagttatctggtaagttcattatctgaaataacatatagattttatctaattaatctaaaatctgatcttgttcaatggagtgctagtacgggcgctgtaattaatcaaatggtaaatactgtattgattacagtgtatgaaaagttacaactggtcatagaaaatgattcacaatttacatgttcattggctgtggaatcaaaacttccaataaaattacttaaagatagaaatgaattatttacaaaattcatcaacgagttaaaaaagaccagttcattcaagataagcaaacgcgataaggatacgctactaaaatattttacttaggactggagttagaatttatagacgactcatttcgtttatcattattagtattcttcttgttatcttgttcagaaatatacagcaatgctatgcctaatactaaatacattatcatgcttgcaatggctctaacaacgacgaaccaaaatgaatttggtcgtagcttttgttcacaaaaatacataaagaaatgtctacataaatctatggcgccattggctacttgaaatagcgccagtcctcctacagattttaatatagctgtataacatgacatttattcatcatcaaaagagacagagtcaccatctgtcatatttagattttttttcatgtgttcaaagtatcctctactcatttcattataatagtttatcatacttagaattttaggacggatcaatgagtaagacttgactagatcgtcagtagtaatttgtgcatcgtctattctgcatccgcttcgtcgaataatgtatagcatcgctttgagattctccatagctatcaagtctttatacaatgacatggaaatatctgtgaatactttatacttctccaacatcgatgccttaacatcatcgcctactttagcattgaaaatacgttctattgtgtagatggatgtagcaagatttttaaacaacaatgccattttacacgatgattgcctcaagtctccaatcgtttgtttagaacgattagctacagagtccaatgcttggctgactagcatattattatctttagaaattgtattcttcaatgaggcgtttatcatatctgtgatttcgttagtcatattacagtctgactgggttgtaatgttatccaacatatcacctatggatacggtacacgtaccagcatttgtaataatcctatctaagatgttgtatggcattgcgcagaaaatatcttctcctgtaatatttccactctcgataaatctactcagattattcttaaatgccttattctctggagaaaagatatcagtgtccatcatttcattaatagtatacgcagaaaagataccacgagtatcaattctatccaagatacttatcggttccgagtcacagataatggtttcctctccttcgggagatcctgcatagaaatatctaggacaatagtttctatactgtctgtaactctgataatctctaaagtcactaactgataccatgaaattgagaagatcaaacgctgaagtaattaatttttctgcctcgtttttactacaactagttttcatcaatgtagtgacgatgtattgtttagttactcttggtctaatactgatgatagagatattattacttcccataatggatcttctagtagtcaccttaaagcccattgatgcaaatagcagatagataaagtcttggtatgactcctttctaatatagtacggactacctttgtcacccaactttatacccacataagccataacaacctctttaatagccgtttcatgaggtttatcagccatgagcctgagtagttggaagaatctcatgaatcctgtctcagaaagtcctatatgcatgatagatttatctttcctgggaaactctcgtatagtcatagatgaaatactcttcaaagtttctgaaataagattagtaacagtcttacctccgactactctaggtaacaaacaaactctaataggtgttttctctgcggagataatatcagaaaggatagagcaataagtagtattattgtgattataaagaccgaatacataacaggtagaatttataaacatcatgtcctgaaggtttttagacttgtattcctcgtaatccataccgtcccaaaacatggatttggtaactttgatagccgtagatctttgttccttcgccaacaggttaaagaaattaataaagaatttgtggtttctacctatgtctacaaattgcacgtttggaagcgccacggttacattcactgcagcattttgaggatcgcgagtatgaagtacgatgttattgtttactggtatatctggaaagaattctaccagtctaggaataagagattgatatcgcatagaaatacaaaagttcataatctcatcatctaagagcattttgttaccattgtaataaatatccactctgtcatatgtataaatgaagtactgttcaaacatgatgagatgtttatatgttggcatagtagtgagatctacgtttggtaatggcaatgtattaagattaactccataatgtctagcagcatctgcgatgttataagcgttgtcaaagcggggtcgatcttgtgctgttatatattgtctaacacctataagattatcaaaatcttgtctgcttaatacaccgttaacaatttttgccttgaattcttttattggtgcattaataacatccttatagaggatgttaaacaaataagtgttatcaaagttaagatctggatatttcttttctgctagaacatccattgagtcggagccatctggtttaatataaccaccgataaatctagctctgtattctgtatccgtcaatctaatattaagaaggtgttgagtgaaaggtggaagatcgtaaaagctgtgagtattaatgataggattagtttccgaactaatgttaattggggtattaataatatctatatttccagcgttaagtgtaacattaaacagttttaattcacgtgacgtggtatcaattaaataattaatgcccaatttggatatagcagcctgaagctcatcttgtttagttacggatcctaatgagttattaagcaatatatcgaacggatgaacgaaggttgttttaagttggtcacatactttgtaatctagacatagatgcggaagaacggtagaaactatacgaaataaatattcagagtcctctaattgatcaagagtaactattgacttaataggcatcatttatttagtattaaatgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattctccggccactattatcgaacgagtacaacctcatactactattattgacgatactccacctcctacgtttcgtagagagttattgatatcggaacaacgtcaacaacgagaaaaaagatttaatattacagtatcgaaaaatgctgaagcaataatggaatctagatctatgatatcttctatgccaacacaaacaccatccttgggagtagtttatgataaagataaaagaattcagatgttggaggatgaagtggttaatcttagaaatcaacgatctaatacaaaatcatctgataatttagataattttaccagaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacgaaaaattatcgtcactaacgtgattattattgtcataaatattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaatttaatacattcccatatccagacaacaatcgtctggattaatctgttcctgtcgtctcataccggacgacatattaatctttttattagtaggcatctttttagatggtttctttttcccagcattaactgagtcgatacctagaagatcgtgattgatctctccgaccattccacgaacttctaattggccgtctctgacggtaccataaactattttaccagcattagtaacagcttggacaatctgaccatccatcgcattgtacgatgtagtagtaactgttgttctacgtctaggagcaccagaagtatttttggagcccttggatgttgatgtagaagaagacgaggattttgattttggtttacatgtaatacattttgtatcacatgcgccggcagtcacatctgtttgagaattaagattattgttgcctcctttgacggctgcatctccaccgatttgcgctagtagatttttaagctgtggtgtaatcttattaactgtttcgatataatcatcgtaactgcttctaacggctaaattttttttatccgccatttagaagctaaaaatatttttatttatgcagaagatttaactagattatacaatgaactaatatgatccttttccagattatttacaaacttggtattttttggttctggaggaggcgaatttaaattcggacttggatttggattttgtgggttcttgatcttattatacagcgcatataggatggcgacggtaactgctacgcaaataccgatcaacaaaagaataccaatcatttattgacaataacttcactattgatcaagtatgcaatatatcatcttttcactaaataagtagtaataatgattcaacaatgtcgagatatatggacgataataatttagttcatggaaatatcgctatgattggtatgaatgactccgctaactctgtggggcgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatatccatttaactaataaaaattttaaatcgccgaatgaacaaagtggaatataaaccatataaaaacaatagtttgtactgcaaaaataatatctatttttgttttcgaagatatggtaaaattaaatagtagtacacagcatgttataactaacagcagcaacggctcgtaattacttatcatttactagacgaaaaggtggtgggatattttcttgctcaaataatacgaatatatcacccatccattttatgcgatgtttatatactctaatctttaatagatctatagacgacgggtttaccaacaatatagattttatcgattcatctaatttaaacccttccttaaacgtgaatgatctattatctggcataacgatgactctacccgatgaatcggacaatgtactgggccatgtagaataaattatcaacgaattatcgtctacgaacatttatatcatttgttttaattttagtacgcgaataaatagatataaaatagaaaataacagatattacaaccaatgttatggccgcgcccaaccaggtaggcagttttattttatcttttactacaggttctcctggatgtacgtcaccaacggcggacgtagttctagtacaattagacgtaagttccgcttgggaattttttaacgctaaagagttaacgttaatcgtgcacccaacgtatttacatctagttctttgaacatcttgattataatataaccattttctatctctagattcgtcggtgcactcatgtaaccaacataccctaggtcctaaatatttatctccggaattagattttggataattcgcgcaccaacaatttctatttcctttatgatcgttacaaaagacgtataatgccgtatccccaaaagtaaaataatcaggacgaataattctaataaactcagaacaatatctcgcatccatatgtttggagcaaatatcggaataagtagacatagccggtttccgttttgcacgtaaccattctaaacaattggggtttccaggatcgtttctacaaaatccagtcatgaaatcatcacaatgttctgtcttgtaattattattaaatatttttggacagtgtttggtatttgtcttagaacaacattttgccacgctatcactatcgcccaggagataatccttttttataaaatgacatcgttgcccggatgctatataatcagtagcgtgttttaaatccttaatatattcaggagttacctcgttctgataatagattaatgatccaggacgaaatttgaaagaactacatggttctccatgaattaatacatattgtttagcaaattcaggaactataaaactactacaatgatctatcgacataccatctatcaaacaaaacttgggtttaatttctcccggagatgtttcataatagtacgtataactttcttctgcaaacttaacagctctattatattcaggataattaaaacctaattccatatatttgtctcgtatatctgctattcctggtgctattttgattctattaagagtaacggctgcccccatttttaataatcgtcagtatttaaactgttaaatgttggtatatcaacatttaccttatttcccgcagtataaggtttgttgcaggtatactgttcaggaatggttacatttatactttttctatagtcctgtctttcgatgttcatcacatatgcaaagaacagaataaacaaaataatgtaagaaataatattaaatatctgtgaattcgtaaatacattgattgccataataattacagcagctacaatacatacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcatttatatattaaaaaatgtcactattaaagatggagtataatctttatgccgaactaaaaaaaatgacttgtggtcaacccctaagtctttttaacgaagacggggatttcgtagaagttgaaccgggatcatcctttaagtttctgatacctaagggattttacgcctctccttccgtaaagacgagtctagtatttgaaacattaacaacgaccgataataaaattactagtatcaatccaacaaatgcgccaaagttatatcctcttcaacgcaaagtcgtatctgaagtagtttctaatatgaggaaaatgatcgaatcaaaacgtcctctatacatcactcttcacttggcgtgtggatttggtaagactattaccacgtgttatcttatggctacacacggtagaaaaaccgtcatttgcgtacccaataaaatgttaatacatcaatggaagacacaggtagaggcagtcggattggaacataagatatccatagatggagtaagtagtctattaaaggaactaaagactcaaagtccggatgtattaatagtagtcagtagacatctgacaaacgatgccttttgtaaatatatcaataagcattatgatttgttcatcttggatgaatcacatacgtataatctgatgaacaatacagcagttacaagatttttagcgtattatcctccgatgatgtgttattttttaactgctacacctagaccatctaacagaatttattgtaacagtattattaatattgccaagttatccgatctaaaaaaaactatctatgcagtagatagtttttttgagccatattccacagataatattagacatatgataaaacgattagatggaccatctaataaatatcatatatataccgagaagttattatctgtagacgagcctagaaatcaacttattcttaataccctggtagaagaattcaagtcaggaactattaatcgcattttagttattactaaactacgtgaacatatggtattcttctacaaacgattattagattttttcggatcagaggttgtatttataggagacgcccaaaatagacgtactccagatatggtcaaatcaatcaaggaactaaatagatttatattcgtatccaccttattttattccggtactggtttagatattcctagtttggattctttgttcatttgctcggcagtaatcaacaatatgcaaatagagcaattactagggagggtatgtcgagaaacagaactattagataggacggtatatgtatttcctagcacatccatcaaagaaataaagtacatgataggaaatttcgttcaacgaattattagtctgtctgtagataaactaggatttaaacaaaaaagttatcggaaacatcaagaatccgatcccacttctgtatgtacaacatcctccagagaagaacgtgtattaaatagaatatttaactcgcaaaatcgttaagaagtttaagcgacgatccgcatgctgcgcaggccagtgtattacccctcatagtattaatataatccaatgatacttttgtgatgtcggaaatcttaaccaatttagactgacaggcagaacacgtcatgcaatcatcatcgtcatcgataactgtagtcttgggcttctttttgcggctcttcattccggaacgcacattggtgctatccatttaggtagtaaaaaataagtcagaatatgccctatagcacgatcgtgcaaaacctggtatatcgtctctatctttatcacaatatagtgtatcgacatctttattattattgacctcgtttatcttggaacatggaatgggaacatttttgttatcaacggccacctttgccttaattccagatgttgtaaaattataactaaacagtctatcatcgacacaaatgaaattcttgtttagacgtttgtagtttacgtatgcggctcgttcgcgtctcattttttcagatattgcaggtactataatattaaaaataagaatgaaataacataggattaaaaataaagttatcatgacttctagcgctgatttaactaacttaaaagaattacttagtctgtacaaaagtttgaaattttcagattctgcggctatagaaaagtataattctttggtagaatggggaacatctacttactggaaaataggcgtgcaaaaggtagctaatgtcgagacgtcaatatctgattattatgatgaggtaaaaaataaaccgtttaatattgatccgggctattacattttcttaccggtatattttgggagcgtctttatttattcgaagggtaaaaatatggtagaacttggatctggaaactcttttcaaataccagatgatatgcgaagtgcgtgtaacaaagtattagacagcgataacggaatagactttctgagatttgttttgttaaacaatagatggataatggaagatgctatatcaaaatatcagtctccagttaatatatttaaactagctagtgagtacggattaaacatacccaaatatttagaaattgaaatagaggaagacacattatttgacgacgagttatactctattatagaacgctctttcgatgataaatttccaaaaatatccatatcgtatattaagttgggagaacttaggcggcaagttgtagactttttcaaattctcattcatgtatattgagtccatcaaggtagatcgtataggagataatatttttattcctagcgttataacaaaatcaggaaaaaagatattagtaaaagatgtagaccatttaatacgatccaaggttagagaacatacatttgtaaaagtaaaaaagaaaaacacattttccattttatacgactatgatggaaacggaacagaaactagaggagaagtaataaaacgaattatagacactataggacgagactattatgttaacggaaagtatttctctaaggttggtagtgcaggcttaaagcaattgactaataaattagatattaatgagtgcgcaactgtcgatgagttagttgatgagattaataaatccggaactgtaaaacgaaaaataaaaaaccaatcagcatttgatttaagcagagaatgtttgggatatccagaagcggattttataacgttagttaataacatgcggttcaaaatagaaaattgtaaggttgtaaatttcaatattgaaaatactaattgtttaaataacccgagtattgagactatatatggaaactttaaccagttcgtctcaatctttaatatcgtcaccgatgtcaaaaaaagattattcgagtgaaataatatgcgcctttgatataggtgcaaaaaatcctgccagaactgttttagaagtcaaggataactccgttagggtattggatatatcaaaattagactggagttctgattgggaaaggcacatagctaaagatttgtcacaatatgaatacactacagttcttctagaacgtcagcctagaaggtcgccgtacgtcaaatttatctattttattaaaggctttttatatcatacatcggctgccaaagttatttgcgtctcgcctgtcatgtctggtaattcatatagagatcgaaaaaagagatcggtcgaagcatttcttgattggatggacacattcggattgcgagactccgttccggatagacgcaaattagacgatgtagcggatagtttcaatttggctatgagatacgtattagataaatggaatactaattatacaccttataataggtgtaaatctagaaattacataaaaaaaatgtaataacgttagtaacgccattatggataatctatttacctttctacatgaaatagaagatagatatgccagaactatttttaactttcatctaataagttgcgatgaaataggagatatatatggtcttatgaaagaacgcatttcctcagaggatatgtttgataatatagtgtataataaagatatacatcatgccattaagaaactagtgtattgcgacatccaacttactaaacacattattaatcagaatacgtatccggtatttaacgattcttcacaagtgaaatgttgtcattatttcgatataaactcagataatagcaatattagctctcgtacagtagagatatttgagagggaaaagtcatctcttgtatcatatattaaaactaccaataagaagagaaaggtcaattatggggaaataaagaaaactgtacatggaggcactaatgcaaattacttttccggtaaaaagtctgatgagtatctgagcactacagtcaggtccaacattaatcaaccttggatcaaaaccatttctaagagaatgagagtagatatcattaatcactctatagtaacgcgtggaaaaagctctatattacaaactatagaaattatttttactaatagaacatgtgtgaaaatattcaaggattctactatgcacattattctatccaaggacaaggatgaaaaggggtgtatacacatgattgacaaattattctatgtctattataatttatttctgttgttcgaggatatcatccaaaacgagtactttaaagaagtagctaatgttgtaaaccacgtactcacggctacggcattagatgagaaattattcctaattaagaaaatggctgaacacgatgtttatggagttagcaatttcaaaatagggatgtttaacctgacatttattaagtcgttggatcataccgttttcccctctctgttagatgaggatagcaaaataaagttttttaaggggaaaaagctcaatattgtagcattacgatctctggaggattgtataaattacgtgactaaatccgagaatatgatagaaatgatgaaggaaagatcgactattttaaatagcatagatatagaaacggaatcggtagatcgtctaaaagaattgcttctaaaatgaaaaaaaaacactaattcagaaatggatcaacgactcggatataagtttttggtgcctgatcctaaagccggagttttttatagaccgttacatttccaatatgtatcgtattctaattttatattgcatcgattgcatgaaatcttgaccgtcaagcggccactcttatcgtttaagaataatacagaacgaattatgatagaaattagcaatgttaaagtgactcctccagattactcacctataatcgcgagtattaaaggtaagagttatgacgcattagccacgttcactgtaaatatctttaaagaggtaatgaccaaagagggtatatccatcactaaaataagtagttatgagggaaaagattctcatttgataaaaattccgctactaataggatacgggaataaaaatccacttgatacagccaagtatcttgttcctaatgtcataggtggagtctttatcaataaacaatctgtcgaaaaagtaggaattaatctagtagaaaagattacaacatggccaaaatttagggttgttaagccaaactcattcactttctcgttttcctccgtatcccctcctaatgtattaccgacaagatatcgccattacaagatatctctggatatatcacaattggaagcgttgaatatatcatcgacaaagacatttataacggtcaatattgttttgctgtctcaatatttatctagagtgagtctagaattcattagacgtagtttatcatacgatatgcctccagaagttgtctatctagtaaacgcgataatagatagtgctaaacgaattactgaatctattactgactttaatattgatacatacattaatgacctggtggaagctgaacacattaaacaaaaatctcagttaacgatcaacgagttcaaatatgaaatgctgcataactttttacctcatatgaactatacacccgatcaactaaagggattttatatgatatctttactaagaaagtttctctactgtatctaccacacttctagatatccagatagagattcgatggtttgtcatcgcatcctaacgtacggcaaatattttgagacgttggcacatgatgaattagagaattacataggcaacatccgaaacgatatcatgaacaatcacaagaacagaggcacttacgcggtaaacattcatgtactaacaactcctggacttaatcatgcattttctagtctattgagtggaaagttcaaaaagtcagacggtagttatcgaacacatcctcactattcatggatgcagaatatttctattcctaggagtgttggattttatccggatcaagtaaagatttcaaagatgttttctgtcagaaaataccatccaagtcaatatctttacttttgttcatcggacgttccggaaagaggtcctcaggtaggtttagtatctcaattgtctgtcttgagttccattacaaatatactaacgtctgagtatttggatttggaaaagaaaatttgtgagtatatcagatcatattataaagatgatataagttactttgaaacaggatttccaatcactatagaaaatgctctagtcgcatctcttaatccaaatatgatatgtgattttgtaactgactttagacgtagaaaacggatgggattcttcggtaacttggaggtaggtattactttagttagggatcacatgaatgaaattcgcattaatattggagcgggaagattagtcagaccattcttggttgtggataacggagagctcatgatggatgtgtgtccggagttagaaagcagattagacgacatgacattctctgacattcagaaagagtttccgcatgtcatcgaaatggtagatatagaacaatttacttttagtaacgtatgtgaatcggttcaaaaatttagaatgatgtcaaaggatgaaagaaagcaatacgatttatgtgactttcctgccgaatttagagatggatatgtagcatcttcactagtgggaatcaatcacaattctggacccagagctattcttggatgtgctcaagctaaacaagctatctcttgtctgagctcggatatacgaaataaaatagacaatggaattcatttgatgtatccagagaggccaatcgtgattagtaaggctttagaaacttcaaagattgcggctaattgcttcggccaacatgttactatagcattaatgtcgtacaaaggtatcaatcaagaggatggaattatcatcaaaaaacaatttattcagagaggcggtctcgatattgttacagccaagaaacatcaagtagaaattccgttggaaaactttaataacaaagaaagagataggtctaacgcctattcaaaattagaaagtaatggattagttagactgaatgctttcttggaatccggagacgctatagcacgaaatatctcatcaagaactcttgaagatgattttgctagagataatcagattagctttgatgtttccgaaaaatataccgatatgtacaaatctcgcgttgaacgagtacaagtagaacttactgacaaagttaaggtacgagtattaaccatgaaagaaagaagacccattctaggagacaaatttaccactagaacgagtcaaaagggaacagtcgcgtatatcgcggatgaaacggaacttccatacgatgaaaatggtatcacaccagatgtcattattaattctacatccatcttctctagaaaaactatatctatgttgatagaggttattttaacagccgcatattctgctaagccgtacaacaataagggagaaaaccgacctgtctgttttcctagtagtaacgaaacatccatcgatacatatatgcaattcgctaaacaatgttatgagcattcaaatccgaaattgtctgatgaagaattatcggataaaatcttttgtgaaaagattctctatgatcctgaaacggataagccttatgcatccaaagtattttttggaccaatttattacttgcgtctgaggcatttaactcaggacaaggcaaccgttagatgtagaggtaaaaagacgaagctcattagacaggcgaatgagggacgaaaacgtggaggaggtatcaagttcggagaaatggagagagactgtttaatagcgcatggtgcagccaatactattacagaagttttgaaagattcggaagaagattatcaagatgtgtatgtttgtgaaaattgtggagacatagcagcacaaatcaagggtattaatacatgtcttagatgttcaaaacttaatctctctcctctcttaacaaaaattgataccacgcacgtatctaaagtatttcttactcaaatgaacgccagaggcgtaaaagttaaattagatttcgaacgaaggcctccttcgttttataaaccattagataaagttgatctcaaaccgtcttttctggtgtaatattctagtttggtagtagatacatatcaatatcatcaaattcgagatccgaattataaaatgggcgtggattgttaactatagaatcggacgtctgatattcgaaaatctgtggagttttaggttttggtggaggtgtaactgctacttgggatactgaagtctgatattcagaaagctgggggatgttctggttcgacatccaccgatggtgtcacatcactaatcggttcggtaacgtctgtggacgatggaggcaccacttctacaggttctggttctttatcctcagtcatcaacggagctacttcaatgcgaggaaatgtataatttggtaatggtttctcatgtggatctgaagaagaggtaagatatctactagaaagataccgatcacgttctagttctcttttgtagaacttaactttttctttctccgcatctagttgatattccaacctcttcacgttcgcatgggttacctccgcagtttttacgagcgatttcacgttcagccttcatgcgtcttatagcatgaattcgcttatcgttatcgggtttagcttctgtcaccttagcaattccttttttattaaactctacataatcatatccatttctattgtttgttctaatataaacgagtatagcatcattgctaaatttttcaatagtatcgaaaacagaatatcctaaaccatataatatatattcaggaacactcaaactaaatgtccaggattctcctaaatacgtaaactttaatagtgcgaaatcattcaaaaatctaccacttatagatagatagatagtacataaatgcgtatagtagtctacctatctctttattatgaaaaccggcattacgatcatatatgtcgtgatatacctgtgatccgtttacgttaaaccataaatacatgggtgatcctataaacatgaatttatttctaattctcagagctatagttaattgaccgtgtaatatttgcttacatgcatacttgatacgatcattaataagatttttatcattgctcgttatttcagaatcgtatatataaggagtaccatcgtgattcttaccagatattatacaaaatactatatataaaatatattgacccacgttagtaatcatgtaaatgtttaacgttttaaattttgtattcaatgatccattatcatacgctagcatggtcttatgatattcattctttaaaatataatattgtgttagccattgcattggggctcctaatggagattttttattctcatccattttaggataggctttcataaagtccctaataacttcgtgaataatgtttctatgttttctactgatgcatgtatttgcttcgatttttttatcccatgtttcatctatcatagatttaaacgcagtaatgctcgcaacattaacatcttgaaccgttggtacaattccgttccataaatttataatgttcgccatttatataactcattttttgaatatacttttaattaacaaaagagttaagttactcatatggacgccgtccagtctgaacatcaatctttttagccagagatatcatagccgctcttagagtttcagcgtgattttccaacctaaatagaacttcatcgttgcgtttacaacacttttctatttgttcaaactttgttgttacattagtaatctttttttccaaattagttagccgttgtttgagagtttcctcattgtcgtcttcatcggctttaacaattgcttcgcgtttagcctctggctttttagcagccgttgtagaaaaaaattcagttgctggaattgcaagatcgtcatctccggggaaaagagttccgtccatttaaagtacagattttagaaactgacactctgcgttatttatatttggtacaacacatggattataaatattgatgttaataacatcagaaaatgtaaagtctatacattgttgcatcgtgttaaattttctaatggatctagtattattgggtccaacttctgcctgaaatccaaatatggaagcggatacaaaaccgtttcctggataaaccacacatctccacttttgctttacatcagaaattgtgtcgttgacatcttgaactctcctatctaatgccggtgttccacctatagattttgaatattcgaatgctgcatgagtagcattaaattccttaatattgccataattttcatatattgagtaaccctggataaaaagtaaacacaccgcagccgtagctaccacaataaaaaaaattgatagagagttcatttataatctattagaagctgacaaaatttttttacacgcatcagacaatgctttaataaatagttcaacatctacttttgtcatatcgaaccgatggtatgattctaacctagaattacatccgaaaaagttgactatgttcatagtcattaagtcattaacaaacaacattccagactctggattataagacgatactgtttcgtcacaattacctaccttaatcatgtgattatgaatattggctattagagcaccttctaagaaatctataatatctttgaaacacgatttaaaatcaaaccacgaatatacttctacgaagaaagttagtttacccataggagaaataactataaatggagatctaaatacaaaatccggatctatgatagttttaacattattatattctctattaaatacctccacatctaaaaatgttaattttgaaactatgtcttcgtttattaccgtacctgaactaaacgctataagctctattgtttgagaactctttaaacgatattcttgaaatacatgtaacaaagtttcctttaactcggtcggtttatctaccatagttacagaatttgtatccttatctataatataataatcaaaatcgtataaagttatataattatcgcgttcagattgggatcttttcaaatagactaaaaaccccatttctctagtaagtatcttatgtatatgtttgtaaaatatcttcatggtgggaatatgctctaccgcagttagccattcctcattgacagcggtagatgtattagacaaaactattccaatgtttaacaagggccattttacgagattattaaatccttgtttgataaatgtagccaatgagggttcgagttcaacgacgattgaattctcttcccgcggatgctgcatgatgaacgacgggatgttgttcgattgatttggaattctttttcgactttttgtttatattaaatattttaaaatttatagcggatagcaattcatgtaccacggataatgtagacgcgtattgcgcatcgatatctttattattagataaatttatcaataaatgtgagaagtttgcctcgttaaggtcttccatttaaatattatataaacatttgtgtttgtatcttattcgtcttttatggaatagttttttactagtaaagctgcaattacacactttgtccgtaaaacataaatataaacaccagcttttatcaatcgttccaaaaagtcgacggcggacatttttaacatggcatctattttaaatacacttaggtttttggaaaaaacatcattttataattgtaacgattcaataactaaagaaaagattaagattaaacataagggaatgtcatttgtattttataagccaaagcattctaccgttgttaaatacttgtctggaggaggtatatatcatgatgatttggttgtattggggaaggtaacaattaatgatctaaagatgatgctattttacatggatttatcatatcatggagtgacaagtagtggaacaatttacaaattgggatcgtctatcgatagactttctctaaataggactattgttacaaaagttaataattataattatgatacattttttgacgatgatgattgatcgctattgcacaattttgtttttttactttctaatatagcgtttagattctttttcatgtgcgaatattgatttactaaaatatctatgtttaacttttgttctataacgtccttatcggcggtatcggtacatatacgtaattcaccttcacaaaatacggagtcttcgataataatagccaatcgattattggatctagctgtctgtatcatattcaacatgtttaatatatcctttcgtttcccctttacaggcatcgatcgtagcatattttccgcgtctgagatggaaatgttaaaactacaaaaatgcgtaatgttagcccgtcctaatattggtacgtgtctataagtttggcatagtagaataatagacgtgtttaaatgccttccaaagtttaagaattctattagagtattgcattttgatagtttatcgcctacatcatcaaaaataagtaaaaagtgtgctgattttttatgattttgtgcgacagcaatacatttttctatgttacttttagttcgtatcagattatattctagagattcctgactactaacgaaattaatatgatttggccaaatgtatccatcataatctggattataaacgggtgtaaacaagaatacatgtttatattttttaactagtgtagaaaacagagatagtaaatagatagtttttccagatccagatcctcccgttaaaaccattctaaacggcatttttaataaattttctcttgaaaattgtttttcttggaaacaattcataattatatttacagttactaaattaatttgataataaatcaaaatatggaaaactaaggtcgttagtagggaggagaacaaagaaggcacatcgtgacataaataacatttattatcatgatgacaccagaaaacgacgaagagcagacatctgtgttctccgctactgtttacagagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgactacctggctcattgattatgttaaggatacatggggatctgatggtaatccaattacaaaaactacatccgattatcaagattctgatgtatcacaagaagttagaaagtatttttgtgttaaaacaatgaactaatatttatttttgtacattaataaatgaaatcgcttaatagacaaactgtaagtaggtttaagaagttgtcggtgccggtcgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtgacaacaaaaaatgaattaataataagtcgttaacgtacgccgccatggacgccgcgtttgttattactccaatgggtgtgttgactataacagatacattgtatgatgatctcgatatctcaatcatggactttataggaccatacattataggtaacataaaaactgtccaaatagatgtacgggatataaaatattccgacatgcaaaaatgctactttagctataagggtaaaatagttcctcaggattctaatgatttggctagattcaacatttatagcatttgtgccgcatacagatcaaaaaataccatcatcatagcatgcgactatgatatcatgttagatatagaagataaacatcagccattttatctattcccatctattgatgtttttaacgctacaatcatagaagcgtataacctgtatacagctggagattatcatctaatcatcaatccttcagataatctgaaaatgaaattgtcgtttaattcttcattctgcatatcagacggcaatggatggatcataattgatgggaaatgcaatagtaattttttatcataaaagttgtaaagtaaataataaaacaataaatattgaactagtagtacgtatattgagcaatcagaaatgatgctggtacctcttatcacggtgaccgtagttgcgggaacaatattagtatgttatatattatatatttgtaggaaaaagatacgtactgtctataatgacaataaaattatcatgacaaaattaaaaaagataaagagttctaattccagcaaatctagtaaatcaactgatagcgaatcagactgggaggatcactgtagtgctatggaacaaaacaatgacgtagataatatttctaggaatgagatattggacgatgatagcttcgctggtagtttaatatgggataacgaatccaatgttatggcgcctagcacagaacacatttacgatagtgttgctggaagcacgctgctaataaataatgatcgtaatgaacagactatttatcagaacactacagtagtacttaatgaagataccaaacagaatcctaactattcatccaatcctttcgtaaattataataaaaccagtatttgtagcaagtcaaatccgttcattacagaactcaacaataaatttagtgagaataatccgtttagacgagcacatagcgatgattatcttaataagcaagaacaagatcatgaacacgatgatatagaatcattggtgtgattagtttcctttttataaaattgaagtaatatttagtattattgctgccgtcacgttgtacaaatggagatattccctgtattcggcatttctaaaattagcaattttattgctaataatgactgtagatattatatagatacagaacatcaaaaaattatatctgatgagatcaatagacagatggatgaaacggtacttcttaccaacatcttaagcgtagaagttgtaaatgacaatgagatgtaccatcttattcctcatagattatcgacgattatactctgtattagttctgtcggaggatgtgttatctctatagataatgacgtcaatggcaaaaatattctaacctttcccattgatcatgctgtaatcatatccccactgagtaaatgtgtcgtagttagcaagggtcctacaaccatattggttgttaaagcggatatacctagcaaacgattggtaacatcgtttacaaacgacatactgtatgtaaacaatctatcactgattaattattcgccgttgtctgtattcattattagacgagttaccgactatttggatagacacatatgcgatcagatatttgcgaataataagtggtattccattataaccatcgacaataagcagtttcctattccatcaaactgtataggtatgtcctctgccaagtacataaattctagcatcgagcaagatactttaatacatgtttgtaacctcgagcatccattcgacttagtatacaaaaaaatgcagtcgtacaattctgtacctatcaaggaacaaatattgtacggtagaattgataatataaatatgagcattagtatttctgtggattaatagatttctagtatggggatcattaatcatctctaatctctaaatacctcataaaacgaaaaaaaagctattatcaaatactgtacggaatggattcattctcttctctttttatgaaactctgttgtatatctactgataaaactggaagcaaaaaatctgataaaaagaataagaataagatcaaggattattataaaataacaatagttcctggttcctcttccacgtctactagctcgtggtattatacacatgcctagtaatagtctctttgcgttgacggaaagcagactagaaataacaggctaaaatgttcagacaccataatagttcccaacccagataataacagagtaccatcaacacattcctttaaactcaatcccaaacccaaaaccgttaaaatgtatccggccaattgatagtagataatgaggtgtacagcgcatgatgatttacacagtaaccaaaatgaaaatactttagtaattataagaaatatagatggtaacgtcatcatcaacaatccaataatatgccggagagtaaacattgacggataaaacaaaaatgctccgcataactctatcatggcaataacacaaccaaatacttgtaagattcctaaattagtagaaaatacaacggatatcgatgtataagtgatctcgagaaataataagaataaagtaatgcccgtaaagataaacatcaacattgtttggtaatcattaaaccaattagtatgaagttgaactaatttcacagtagattttattccagtattatccccgcatgtataagtacctggtaagatatctttatattccataatcaatgagacatcactatctgataacgaatgaagtctagcactagtatgccatttacttaatattgtcgtcttggaagttttattataagttaaaatatcatggttatccaatttccatctaatatactttgtcggattatctatagtacacggaataatgatggtatcattacatgctgtatactctatggtctttgtagttgttataacaaccaacgtatagaggtatatcaacgatattctaactcttgacattttttatttatttaaaatgatacctttgttatttattttattctattttgctaacggtattgaatggcataagtttgaaacgagtgaagaaataatttctacttacttattagacgacgtattatacacgggtgttaatggggcggtatacacattttcaaataataaactaaacaaaactggtttaactaataataattatataacaacatctataaaagtagaggatgcggaaccaataacggaaatcccaaatgttggaaaatagacggttcagacgacccaaaacatagaggtagaggatacgctccttatcaaaatagcaaagtaacgataatcagtcacaacggatgtgtactatctgacataaacatatcaaaagaaggaattaaacgatggagaagatttgacggaccatgtggttatgatttatacacggcggataacgtaattccaaaagatggtttacgaggagcattcgtcgataaagatggtacttatgacaaagtttacattcttttcactgatactatcggctcaaagagaattgtcaaaattccgtatatagcacaaatgtgcctaaacgacgaaggtggtccatcatcattgtctagtcatagatggtcgacgtttctcaaagtcgaattagaatgtgatatcgacggaagaagttatagacaaattattcattctagaactataaaaacagataatgatacgatactatatgtattcttcgatagtccttattccaagtccgcattatgtacctattctatgaataccattaaacaatctttttctacgtcaaaattggaaggatatacaaagcaattgccgtctccagctcctggtatatgtttaccagctggaaaagttgttccacataccacgtttgaagtcatagaaaaatataatgtactagatgatattataaagcctttatctaaccaacctatcttcgaaggaccgtctggtgttaaatggttcgatataaaggagaaggaaaatgaacatcgggaatatagaatatacttcataaaagaaaattctatatattcgttcgatacaaaatctaaacaaactcgtagctcgcaagtcgatgcgcgactattttcagtaatggtaacttcgaaaccgttatttatagcagatatagggataggagtaggaatgccacaaatgaaaaaaatacttaaaatgtaatcttaatcgagtacaccgcacgacaatgaacaaacataagacagattatgctggttatgcttgctgcgtaatatgcggtctaattgttggaattatttttacagcgacactattaaaagttgtagaacgtaaattagttcatacaccatcaatagataaaacgataaaagatgcatatattagagaagattgtcctactgactggataagctataataataaatgtatccatttatctactgatcgaaaaacctgggaggaaggacgtaatgcatgcaaagctctaaatccaaattcggatctaattaagatagagactccaaacgagttaagttttttaagaagcattagacgcggatattgggtaggagaatccgaaatattaaaccagacaaccccatataattttatagctaaaaatgccacgaagaatggaactaaaaaacggaaatatatttgtagtacaacgaatactcccaaactacatttttatcataccactacttcggttagatgttttagaaaaaaataaatatcgccgtaccgttcttgtttttataaaaataacaattaacaattatcaaattttttctttaatattttacgtggttgaccattcttggtggtaaaataatctcttagtgttggaatggaatgctgtttaatgtttccgcactcatcgtatattttgacgtatgcagtcacatcgtttacgcaatagtcagactgtagttctatcatgcttcctacatcagaaggaggaacagttttaaagtctcttggttttaatctattgccattagttttcatgaaatcctttgttttatccacttcacattttaaataaatgtccactatacattcttctgttaattttactagatcgtcatgggtcatagaatttataggttccgtagtccatggatccaaactagcaaacttcgcgtatacggtatcgcgattagtgtatacaccaactgtatgaaaattaagaaaacagtttaataaatcaacagaaatatttaatcctccgtttgatacagatgcgccatatttatggatttcggattcacacgttgtttgtctgaggtgttcgtctagtgttgcttctacgtaaacttcgattcccatatattctttattgtcagaatcgcataccgatttatcatcatacactgtttgaaaactaaatggtatacacatcaaaataataaataataacgagtacattctgcaatattgttatcgtaattggaaaaatagtgttcgagtgagttggattatgtgagtattggattgtatattttattttatattttatattttgtagtaagaatagaatgctaatgtcaagtttattccaatagatgtcttattaaaaaacatatataataaataacaatggctgaatggcataaaattatcgaggatatctcaaaaaataataagttcgaggatgccgccatcgttgattacaagactacaaagaatgttctagctgctattcctaacagaacatttgccaagattaatcctctcatcactaatcgtaatattctaaaacctcttattggtcagaaatattgtattgtatatactaactctctaatggatgagaacacgtatgctatggagttgcttactgggtacgcccctgtatctccgatcgttatagcgagaactcataccgcacttatatttttgatgggtaagccaacaacatccagacgtgacgtgtatagaacgtgtagagatcacgctacccgtgtacgtgcaactggtaattaaaataaaaagtaatattcatatgtagtgtcaattttaaatgatgatgatgaaatggataatatccatattgacgatgtcaataatgccggtattggcatacagttcatcgatttttagatttcattcagaggatgtggaattatgttatgggcatttgtattttgataggatctataatgtagtaaatataaaatataatccgcatattccatatagatataattttattaatcgcacgttaaccgtagatgaactagacgataatgtcttttttacacatggttattttttaaaacacaaatatggttcacttaatcctagtttgattgtctcattatcaggaaacttaaaatataatgatatacaatgctcagtaaatgtatcgtgtctcattaaaaatttggcaacgagtacatctactatattaacatctaaacataagacttattctctacatcggtccacgtgtattactataataggatacgattctattatatggtataaagatataaatgacatctatgattttactgcaatatgtatgctaatagcgtctacattgatagtgaccatatacgtgtttaaaaaaataaaaatgaactcttaattatgctatgctattagaaatggataaaatcaaaattacggttgattcaaaaattggtaatgttgttaccatatcgtataacttggaaaagataactattgatgtcacacctaaaaagaaaaaagaaaaggatgtattattagcgcaatcagttgctgtcgaagaggcaaaagatgtcaaggtagaagaaaaaaatattatcgatattgaagatgacgatgatatggatgtagaaagcgcataatacgatctataaaaataagtatataaatactttttatttactgtactcttactgtgtagtggtgataccctactcgattatttttttaaaaaaaaatacttattctgattcttctagccatttccgtgttcgttcgaatgccacatcgacgttaaagataggggagtagttgaaatctagttctgcattgttggtacgcacctcaaatgtagtgttggatatcttcaacgtatagttgttgagtagtgatggttttctaaatagaattctcttcatatcattcttgcacgcgtacatttttagcatccatcttggaatcctagatccttgttctattcccaatggtttcatcaatagaagattaaacatatcgtacgaacacgatggagagtaatcgtagcaaaagtaagcatttcctttaatctcagatcccggatactggatatattttgcagccaacacgtgcatccatgcagcatttcctacatatacccggctatgtaccgcgttatcatcgactgtacgatacataatgttaccgtgttgcttacattgctcgtaaaagactttcatcaatttgtctccttctccgtaaattccagtgggtcttaggcaacaagtatacaattttgctccattcatgattacggaattattggctttcataaccagttgctcggccatacgtttactttttgcgtatacatgtcctggtgatatatcataaagggtatgctcatggccgatgaatggatcaccgtgtttattgggtcctattgcttccatgctactagtatagatcaaatacttgattcctaggtccacacaagctgccaatatagtctgtgttccataatagtttactttcatgatttcattatcggtgtattttccaaatacatccactagagcagctgtatgaataatcagatttaccccatctagcgcttctcttaccttatcaaagtcgtttatatcacattgtatatagtttataaccttaactttcgaggttattggttgtggatcttctacaatatctatgactctgatttcttgaacatcatctgcactaattaacagttttactatatacctgcctagaaatccggcaccaccagtaaccgcgtacacggccattgctgccactcataatatcagactacttattctattttactaaataatggctgtttgtataatagaccacgataatatcagaggagttatttactttgaaccagtccatggaaaagataaagttattggattaaaatccggaacgtatagtttgataattcatcgttacggagatattagtcaaggatgtgattccataggcagtccagaaatatttatcggtaacatctttgtaaacagatatggtgtagcatatgtttatttagatacagatgtaaatatatctacaattattggaaaggcgttatctatttcaaaaaatgatcagagattagcgtgtggagttattggtatttcttacataaatgaaaagataatacattttcttacaattaacgagaatggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgactccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaaatataggtgtatggcattagccgaacatatatctgatagtattccaatatctgagctatctagactacgatacaatctatgtaagtatctacgcgggcacactgagagcatagaggatgaatttgattattttgaagacgatgattcgtctacatgttctgccgtaaccgacagggaaacggatgtataattttttttatagcgtgaaggatatgataaaaaatataattgttgtatttatcccattccaatcaccttatatgattctgtaacacaatgaaggagtcttatagatgtatagaggtcagatactggtttgataaactgtttattccacataagtatgtttgactttatggttagacccgcatactttaacaaatcactgaaaattggagttaggtattgacctctcagaatcagttgccgttctggaacattaaatgtattttttatgatatactccaacgcatttatgtgggcatacaacaagtcattactaatggagtattccaagagaagagatttcaacagactgtttatgaactcgaatgccgcctcattgtcgcttatattgatgatgtcgaattctcccaatatcatcaccgatgagtagctcatcttgttatcgggatccaagttttctaaagatgtcattaaaccctcgatcatgaatggatttatcatcatcgtttttatgttggacatgagcttagtccgtttgtccacatctatagacgacgatttctgaattatttcatatatccctctctttaactccaggaacttgtcaggatggtctactttaatatgttctcgtctaagagatgaaaatctttggatggttgcacgcgacttttctctaaaggatcctctcttaaatgaatccatcttatccttggacaagatggacagtctattttccttagatggtttaatatttttgttacccatgatctataaaggtagacctaatcgtctcggatgaccatatatttattttcagttttattatacgcataaattgtaaaaaatatgttaggtttacaaaaatgtctcgtggggcattaatcgtttttgaaggattggacaaatctggaaaaacaacacaatgtatgaacatcatggaatctataccggcaaacacgataaaatatcttaactttcctcagagatccactgtcactggaaagatgatagatgactatctaactcgtaaaaaaacctataatgatcatatagttaatctattattttgtgcaaatagatgggagtttgcatcttttatacaagaacaactagaacagggaattactttaatagttgatagatacgcattctctggagtagcgtatgccgccgctaaaggcgcgtcaatgactctcagtaagagttatgaatctggattgcctaaacccgacttagttatattcttggaatctggtagcaaagaaattaatagaaacgtcggcgaggaaatttatgaagatgttacattccaacaaaaggtattacaagaatataaaaaaatgattgaagaaggagatattcattggcaaattatttcttctgaattcgaggaagatgtaaagaaggagttgattaagaatatagttatagaggctatacacacggttactggaccagtggggcaactgtggatgtaatagtgaaattacattttttataaatggatgaagcatattactctggcaacttggaatcagtactcggatacgtgtccgatatgcataccgaactcgcatcaatatctcaattagttattgccaagatagaaactatagataatgatatattaaacaaggacattgtaaattttatcatgtgtagatcaaacttggataatccatttatctctttcctagatactgtatatactattatagatcaagagatctatcagaccgaattgattaattcattagacgacaatgaaattatcgattgtatagttaacaagtttatgagcttttataaggataacctagaaaatatagtagatgctatcattactctaaaatatataatgaataatccagattttaaaactacgtatgccgaagtactcggttccagaatagcggatatagatattaaacaagtgatacgtgagaatatactacaattgtctaataatatccgcgaacgatatttgtgaaaatattaaaaaaaaatactttttttattaaatgacgtcgcttcgcgaatttagaaaattatgctgtgatatatatcacgcatcaggatataaagaaaaatctaaattaattagagactttataacagatagggatgataaatatttgatcattaagctattgcttcccggattagacgatagaatttataacatgaacgataaacaaattataaaattatatagtataatatttaaacaatctcaggaagatatgctacaagatttaggatacggatatataggagacactattaggactttcttcaaagagaacacagaaatccgtccacgagataaaagcattttaactttagaagaagtggatagttttttaactacgttatcatccgtaactaaagaatcgcatcaaataaaattattgactgatatcgcatccgtttgtacatgtaatgatttaaaatgtgtagtcatgcttattgataaagatctaaaaattaaagcgggccctcggtacgtacttaacgctattagtcctcatgcctatgatgtgtttagaaaatctaataacttgaaagagataatagaaaatgcatctaaacaaaatctagactctatatctatttctgttatgactccaattaatcccatgttagcggaatcgtgtgattctgtcaataaggcgtttaaaaaatttccatcaggaatgtttgcggaagtcaaatacgatggtgaaagagtacaagttcataaaaataataacgagtttgccttctttagtagaaacatgaaaccagtactctctcataaagtggattatctcaaagaatacataccgaaagcatttaaaaaagctacgtctatcgtattggattctgaaattgttcttgtagacgaacataatgtaccgctcccgtttggaagtttaggtatacacaaaaagaaagaatataaaaactctaacatgtgtttgttcgtgtttgactgtttgtactttgatggattcgatatgacggacattccattgtacgaacgaagatcttttctcaaagatgttatggttgaaatacccaatagaatagtattctcagagttgacgaatattagtaacgagtctcagttaactgacgtattggatgatgcactaacgagaaaattagaaggattggtcttaaaagatattaatggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatagttacatacaaattaaaataacactatttagttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggtaaaaaattgaaaataaatacaaaggttcttgagggttgtgttaaattgaaagcgagaaataatcataaataagataaacgccgccaccatgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaggcgcgcctttttatactagtatttggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagttacagacactgtctcatacactacagtaagtacatcatctggaattgtcactactaaatcaaccaccgatgatgcggatctttatgatacgtacaatgataatgatacagtaccatcaactactgtaggcggtagtacaacctctattagcaattataaaaccaaggactttgtagaaatatttggtattaccgcattaattatattgtcggccgtggcaatattctgtattacatattatatatataataaacgttcacgtaaatacaaaacagagaacaaagtctagatttttgacttacataaatgtctgggatagtaaaatctatcatattgagcgggccatctggtttaggaaagacagccatagccaaaagactatgggaatatatttggatttgtggtgtcccataccactagatttcctcgtcctatggaacgagaaggtgtcgattaccattacgttaacagagaggccatctggaagggaatagccgccggaacatactgagtttttaggaaatatttacggaacttctaaaactgctgtgaatacagcggctattaataatcgtatttgtgtgatggatctaaacatcgacggtgttagaagttttaaaaatacttacctaatgccttactcggtgtatataagacctacctctcttaaaatggttgagaccaagcttcgttgtagaaacactgaagcggatgatgagattcatcgtcgtgtgatgttggcaaaaactgacatggatgaggcaggtgaagccggtctattcgacactattatcattgaagatgatgtgaatttagcatatagtaagttaattcagatactacaggaccgtattagaatgtattttaacactaattagagacttaagacttaaaacttgataattaataatataactcgtttttatatgtggctatttcaacgtctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggatttggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtctaatggcgaacatgttccatttataagaaatccaaataaaatggataacggtactctagaatttacacctatagattcgcataaaggatacgttgtatctagacgtggagatctagaaacacttggatattgtatgattagatggttgggaggtatcttgccatggactaagatatctgaaacaaagaattgtgcattagtaagtgccacaaaacagaaatatgttaacaatactgcgactttgttaatgaccagtttgcaatatgcacctagagaattgctgcaatatattaccatggtaaactctttgacatattttgaggaacccaattacgacaagtttcggcacatattaatgcagggtgtatattattaagtgtggtgtttggttgatgtaaaatttttgtcgataaaaattaaaaaataacttaatttattattgatctcgtgtgtacaaccgaaatcatggcgatgttttacgcacacgctctcggtgggtacgacgagaatcttcatgcctttcctggaatatcatcgactgttgccaatgatgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcacatatcgccacaaaaactactatgctctaagcggaatcgggtacgagagtctagacttgtgtttggaaggagtagggattcatcatcacgtacttgaaacagaaaacgctgtatatggaaaagttcaacatgattattctactatcaaagagaaggccaaagaaatgaatgcactcagttcaggacctatcatcgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaaccacctctctggaagacagtgtgaatcatgtactcatgaaacgtttggaatctatacgccatatgtggtctgttgtatatgatcattttgatattgtgaatggtaaagaatgctgttatgtgcatacgcatttgtctaatcaaaatcttataccgagtactgtaaaaacaaatttgtacatgaagactatgggatcatgcattcaaatgagtatcttagcgaactgaaggaatcaggtggatggagtcccagaccagaaatgcaggaatttgaatatccagatggagtggaagacactgaatcaattgagagattggtagaggagttcttcaatagatcagaacttcaggctggtaaatctattaatgttaaacatacatctgtttcagctaagcaactaagaacacgtatactctcatcttttgccaacacagagggtggatatttgttcattggagttgataataatacacacaaagtatttggattcacggtgggttacgactacctcagactgatagagaatgatatagaaaagcatatcaaaagactttgtgttgtgtatttctgtgagaagaaagaggacatcaagtacacgtgtcgattcatcaaggtatataaacctggggatgaggctacctcgacatacgtgtgcgctatcaaagtggaaagatgctgttgtgctgtgtttgcagattggccagaatcatggtatcaagaagtattctccagatgaataggtgtcacatataaaattttaattaatgtaactatagagaacaaataataggttgtaatatcatatagacaataactaacaattaattagtaactgttatctctttttaactaactaactatacctattaatacatcgtaattatagttcttaacatctattaatcattgattcgcttctttaattttttataaaccaacattgttaattgaaaagggataacatgttacagaatataaattatatatggattttttttaaaaaggaaatacttgactggagtgtatatttatctcttcattatatagcacgcgtgttttccaatttttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcaggttatcatcatccattaatataaaagaatatgtatatcaattagttaataatgatcatccagataataggataagactaatgcttgaaaatggacgtagaacaagacattttttgtcctatatatcagatacagttaatatctatatatgtattttatatagatgccgaagacagttacggttgtacattattacatagatgtatatatcactataagaaatcagaatcagaatcagaatcatacaatgaattaattaagatattgttaaataatggatcagatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaataaattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatttacgacgctgtcagttataatgcgtataatacgttggtctatctattaaacagaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaacataaacagcataatgcagatttattgaaaatgtgtataaaatatactgcgtgtatgaccgattatgatactcttatagatgtacaatcactacagcaatataaatggtatattttaagatgtttcgatgaaatagatatcatgaagagatgttatataaaaaataaaactgtattccaattagttttttgtatcaaagacattaatactttaatgagatacggtaaacatccttctttcgtgaagtgcactagtctcgacgtatacggaagtcgtgtacgtaatatcatagcatctattagatatcgtcagagattaattagtctattatccaagaagctggatcctggagataaatggtcgtgttttcctaacgaaataaaatataacgataacgaactgtccacatatctaaaaatcttataaacactattaaaatataaaatcacactacatcattgtttccttttagtgctcgacagtgtatactatttttaacgctcataaataaaaatgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatataactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtgaatataaatccgttaaaataatgaataattaataattaataatttaataacaaacaagtatcaaaagattaaagacttatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccatgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggaccaccgactgtaacattaactgaatacgacgaccatccgtatgctactagaggtagcaaaaagattcctatttacaaacgcggtgacatgtgtgatatctacttgttgtatacggctaacttcacattcggagattctaaagaaccagtaccatatgatatcgatgactacgattgcacgtctacaggttgcagcatagactttgtcacaacagaaaaagtgtgcgtgacagcacagggagccacagaagggtttctcgaaaaaattactccatggagttcgaaagtatgtctgacacctaaaaagagtgtatatacatgcgcaattagatccaaagaagatgttcccaatttcaaggacaaaatggccagagttatcaagagaaaatttaactaaatttctcggtagcacatcaaatgatgttaccacttttcttagcatgcttaacttgactaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacgatgtgagaagactgaagaggaaacatggggattgaaaatagggttgtgtataattgccaaagatttctatcccgaaagaactgattgcagtgttcatctcccaactgcaagtgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtctccccgctataaaggcattatcaacttctccttattgattgaaaatgaaaatataaatagtttttatgtatagcagtattaccctatagttttattgcttactactaacatggatacagatacagatgttacaaatgtagaagatatcatgaatgaaatagatagagagaaagaagaaatactaaaaaatgtagaaattgaaaataataaaaacattaacaagaatcatcccaatgaatatattagagaagcactcgttattaatacaagtagtaatagtgattccattgataaagaagttatagaatgtatcagtcacgatgtaggaatatagatcatatctactaatttttataatcaatacaaaacataaaaaacaactcgttattacatagcaggcatggaatccttcaagtattgttttgataacgatggcaagaaatggattatcggaaatactttatattctggtaattcaatactctataaggtcagaaaaaatttcactagttcgttctacaattacgtaatgaagatagatcacaaatcacacaagccattgttgtctgaaatacgattctatatatctgtattggatcctttgactatcgacaactggacacgggaacgtggtataaagtatttggctattccagatctgtatggaattggagaaaccgatgattatatgttcttcgttataaagaatttgggaagagtattcgccccaaaggatactgaatcagtcttcgaagcatgcgtcactatgataaacacgttagagtttatacactctcgaggatttacccatggaaaaatagaaccgaggaatatactgattagaaataaacgtctttcactaattgactattctagaactaacaaactatacaagagtggaaactcacatatagattacaacgaggacatgataacttcaggaaatatcaattatatgtgtgtagacaatcatcttggagcaacagtttcaaaacgaggagatttagaaatgttgggatattgcatgatagaatggttcggtggcaaacttccatggaaaaacgaaagtagtataaaagtaataaaacaaaaaaaagaatataaaaaatttatagctactttctttgaggactgttttcctgaaggaaatgaacctctggaattagttagatatatagaattagtatacacgttagattattctcaaactcctaattatgacagactacgtagactgtttatacaagattgaaatattctttttttatagagtgtggtagtgttacggatatctaatattaatattagactatctctatcgcgctacacgaccaatatcgattactatggatatcttctatgaaaggagagaatgtattcatttctccagcgtcaatctcgtcagtattgacaatactgtattatggagctaatggatccactgctgaacagctatcaaaatatgtagaaaaggaggagaacacggataaggttagcgctcagaatatctcattcaaatccatgaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaacaattgtctcctagcaattagtgccgtatactttaaagcaaaatggttgacgccattcgaaaaggaatttaccagtgattatcccttttacgtatcaccaacggaaatggtagacgtaagtatgatgtctatgtacggcgagctatttaatcacgcatctgtaaaagaatcattcggtaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggctcgtataatctggtggatactctagtaaagtcaggactgacagaggtgttcggttcaactggagattatagcaatatgtgtaatttagatgtgagtgtcgacgctatgatccacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaatttttttcgttggtagatattgctctccgacaactaattgttaaccattttttttaaaaaaatagaaaaaacatgtggtattagtgcaggtcgttattcttccaattgcaattggtaagatgacggccaactttagtacccacgtcttttcaccacagcactgtggatgtgacagactgaccagtattgatgacgtcaaacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgtagaaatcatccgttgttatataccggatgagcaaaaaaccatcatcggactttgtgcatatgctgctacttactggggaggtgaagaccatcccactagtaacagtctgaacgcattgtttgtgatgcttgagatgctaaattacgtggattataacatcatattccggcgtatgaattgatgagttgtacatcttgacattttcttctttcttctcttctccctttcccagaaacaaactttttttacccactataaaataaaatgagtatactacctgttatatttctttctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcgggcatcgacgccttagaaataagagacttaaacaacggacacctggaattattaccatagaagatgttagaaaaaatgatgctggttattatacatgtgttttagaatatatatacggtggcaaaacatataacgtaaccagaattgtaaaattagaggtacgggataaaataataccttctactatgcaattaccagaaggtgttgtaacttcaataggtagtaatttgactattgcgtgtagagtatcgttgagacctcccacaacggatgcagacgtcttttggataagtaatggtatgtattacgaagaagatgatggggacggagacggtagaataagtgtagcaaataaaatctatatgaccgataagagacgtgttattacatcccggttaaacattaatcctgtcaaggaagaagatgctacaacgtttacgtgtatggcgtttactattcctagcatcagcaaaacagttactgttagtataacgtgaatgtatgttgttacatttccatgtcaattgagtttataagaatttttatacattatcttccaacaaacaattgacgaacgtattgctatgattaactcccacgatactatgcatattattaatcattaacttgcagactatacctagtgctattttgacatactcatgttcttgtgtaattgcggtatctatattattaaagtacgtaaatctagctatagttttattatttaattttagataatataccgtctccttatttttaaaaattgccacatcctttattaaatcatgaatgggaatttctatgtcatcgttaatatattgtgaacaacaagagcagatatctataggaaagggtggaatgcgatacattgatctatgtagttttaaaacacacgcgaactttgaagaatttatataaatcattccatcgatacatccttctatgttgacatgtatatatccaggaattcttttattaatgtcaggaaatgtataaactaaaacattgcccgaaagcggtgcctctatctgcgttatatccgttcttaacttacaaaatgtaaccaatacctttgcatgacttgttttgttcggcaacgttagtttaaacttgacgaatggattaattacaatagcatgatccgcgcatctattaagtttttttactttaacgcccttgtatgtttttacagagactttatctaaatttctagtgcttgtatgtgttataaatataacgggatatagaactgaatcacctaccttagatacccaattacattttatcagatccagataataaacaaattttgtcgccctaactaattctatattgttatatattttacaattggttatgatatcatgtaataacttggagtctaacgcgcatcgtcgtacgtttatacaattgtgatttagtgtagtatatctacacatgtatttttccgcactatagtattctggactagtgataaaactatcgttatatctgtcttcaatgaactcatcgagatattgctctctgtcatattcatacacctgcataaactttctagacatcttacaatccgtgttattttaggatcatatttacatatttacgggtatatcaaagatgttagattagttaatgggaatcgtctataataatgaatattaaacaattatatgaggacttttaccacaaagcatcataaaaatgagtcgtcgtctgatttatgttttaaatatcaaccgcgaatcaactcataaaatacaagagaatgaaatatatacatattttagtcattgcaatatagaccatacttctacagaacttgattttgtagttaaaaactatgatctaaacagacgacaacctgtaactgggtatactgcactacactgctatttgtataataattactttacaaacgatgtactgaagatattattaaatcatggagtggatgtaacgatgaaaaccagtagcggacgtatgcctgtttatatattgcttactagatgttgtaatatttcacatgatgtagtgatagatatgatagacaaagataaaaaccacttatcgcatagagactattccaacctactactagagtatataaaatctcgttacatgttattgaaggaagaggatatcgatgagaacatagtatccactttattagataagggaatcgatcctaactttaaacaagacggatatacagcgttacattattattatttgtgtctcgcacacgtttataaaccaggtgagtgtagaaaaccgataacgataaaaaaggccaagcgaattatttctttgtttatacaacatggagctaatctaaacgcgttagataattgtggtaatacaccattccatttgtatcttagtattgaaatgtgtaataatattcatatgactaaaatgctgttgacttttaatccgaatttcaaaatatgtaataatcatggattaacgcctatactatgttatataacttccgactacatacaacacgatattcttgttatgttaatacatcactatgaaacaaatgttggagaaatgccgatagatgagcgtcgtataatcgtattcgagtttatcaaaacatattctacacgtccggcagattcgataacttatttgatgaataggtttaaaaatatagatatttatacccgctatgaaggaaagacattattacacgtagcatgtgaatataataatacacacgtaatagattatcttatacgtatcaacggagatataaatgcgttaaccgacaataacaaacacgctacacaactcattatagataacaaagaaaattccccatataccattaattgtttactgtatatacttagatatattgtagataagaatgtgataagatcgttggtggatcaacttccatctctacctatcttcgatataaaatcatttgagaaattcatatcctactgtatacttttagatgacacattttacaatagacacgttaggaatcgcaattctaaaacgtatcgatacgcattttcaaaatacatgtcgtttgataaatacgatggtataataactaaatgtcataaagaaacaatattgctcaaactatccactgttctagacactacactatatgcagttttaagatgccataattcgaaaaagttaagaagatacctcaacgagttaaaaaaatataataacgataagtcctttaaaatatattctaatattatgaatgagagataccttaatgtatattataaagatatgtacgtgtcaaaggtatatgataaactatttcctgttttcacagataaaaattgtctactaacattactaccttcagaaattatatacgaaatattatacatgctgacaattaacgatctttataatatatcgtatccacctaccaaagtatagttgtatttttctcatgcgatgtgtgtaaaaaaactgatattatataaatattttagtgccgtataatgaagatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggagaggctagaaaaaaatagacggcgacaggtttctaataaacgtgttaaacatggtgatttatggatagccaactatacatctaaattcagtaaccgtaggtatttgtgtaccgtaactacaaagaatggtgactgtgttcagggtatagttagatctcatattaaaaaacctccttcatgcattccaaaaacatatgaactaggtactcatgataagtatggcatagacttatactgtggaattctttacgcaaaacattataataatataacttggtataaagataataaggaaattaatatcgacgatattaagtattcacaaacgggaaagaaattaattattcataatccagagttagaagatagtggaagatacaactgttacgttcattacgacgacgttagaatcaagatgtaaaatacttacggttataccgtcgcaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtatcaccgaggcgaccttgtactttgaaaatgttactgaagaatatataggtaatacatataaatgtcgtggacacaactattattttgaaaaaacccttacaactacagtagtattggagtaaatacacaatgcatttttatatacattactgaataattattattattatttatatcgtatttgtgctataacgcgactatctaggtatttgtatctcaccgatagagaacatataaatgtagactctattaaacagttgtgtaaaatatcagatcctaatagatgtggatgtacggctttagaaatgagttcattaaaatatgtgatatcaacggaacatatttatataattatactattgctgttagtataattattgattccacggaagaactaccaacagttactccaattacaacaacatataattatactatcgatgatagcactactgaagaactacaagtgactcctcatatggatctccatcgatgatacatgtattaaaatactttccgaataagtcttttaaatattgtattaattatgaaaaactatgctatgcgagtatgatacgatactagattttatctctagcgagagatgtcgttagaatcatttatcaacgaatatcgataacatgtgtcatttatacgttaaagtctgtccgtcttctctattgtttagactgtttgtagaatgctgtgatataaacaaactagtagacacaaatatttaactcatgatgaagttgagaatgatatgctttagctaatataaaaatatattaatccactatatattctagacttgatttaaaaccgataaactactactacgtactgtataagttaggagcagaccctaattatgtagatgatagaggtaatacttctgcatctatatgtccacttatgagaaaacgtcatttaataagatgcatcgtgaaaagaaatttattaaagagttggtaaaatatgaaaccgaaagtaaataatataggaaatacacctctacataactacgtatctcaatatgatatcactctcattcctcatccacaacccattaaaaaatggaaattaaagccctctattagcataaacggctacaggtctacctttacaatggcctttccttgtgcccagttcagaccctgtcattgccacgctactaaggactccctgaataccgtggccgacgtcagacattgtctgactgaatacatcctgtgggtttctcatagatggacccatagagaaagcgcagggtctctctacaggcttctcatctctttcagaactgatgcaacggagctctttggtggtgagttgaaggattcacttccgtggagatcattaaatgactccatgaaaaccgccgaagaacttcgtgcaatcattggactttgtactcaatcagctatcgtctctggaagagtcttcaacgataagtatatcgacatactacttatgctgcgaaagattctgaacgagaacgactatctcaccctcttggatcatatccgcactgctaaatactaaatctccttcatgctctctcactacactttttatcatcttatgaggaataattagcaccagaatagctatggattgcacatgtattctatgtcgtctactggatgaagatgtgacgtacaaaaaaataaaactagaaattgaaacgtgtcacaacttatcaaaacatatagatagacgaggaaacaatgcgctacattgttacgtctccaataaatgcgatacagacattaagattgttcgactgttactctctcgcggagtcgagagactttgtagaaacaacgaaggattaactccgctaggagcatacagtaagcatagatacgtaaaatctcagattgtgcatctactgatatccagctattcgaattcctctaacgaactcaagtcgaatataaatgatttcgacttacgtctgctaaaatacctaattgtggataaacggatacgtccgtccaagaatacgaattatgcaatcaatggtctcggattggtggatatatacgtaacgacgcctaatccgagaccagaagtattgctatggcttcttaaatcagaatgttacagcaccggttacgtatttcgtacctgtatgtacaacagtgatatgtgtaagaactctcttcattactatatatcgtctcatagagaatctctatccaaggatgtaattaaatgtttgatcgataacaatgtttccatccaatactactggtcttgctcaaccatagatatagagattattaataaaggatgtggacacgtgtagagtatacgacgtcagccctatattagaggcgtattatctaaacaagcgatttagagtaaccccatataatgtagacatggaaatcgttaatcttcttattgagagacgtcatactcttgtcgacgtaatgcgtagtattacttcgtacgattccagagaatataaccactacatcatcgataacattctaaagagatttagacaacaggatgaatccatcgtacaagccatactgataaactacttacattacggcgatatggtaagtatacctatcattcaatgcatgttggataagacgacggacaacaactttgttaataataatctcgtcgatgtaaacgtcgtaaggtttatcgtggaaaatatggacacgcggctgtaaatcacatatctaacaatggccgtctatgtatgtacggtctgatattatcgagatttaataattgcgggtatcactgttatgaagatgtatttgatatactaagcaagtacatggatgatatagatatgatcgataactctactatattacgcggtcgatgtcaataatatacaatttgcaaagcggttattggaatatggagcgagtgtcacgctcgataatcaatacggccatccagaaaagcagttaccaaagagaagctagttgatttattactgagttaccatcccactctagagactatgattgacgcatttaatagagatatacgctatctatatcctgaaccattattcgcctgtatcagatacgccttaatcctagatgatgattttccttctaaagtaagtatgatatcgccggtcgtcataaggaactaaagcgctatagagcagacattaatagaatgaagaatgcctacatatcaggcgtctccatgtttgatatattatttaaacgaagcaaacgccacagattgagatacgcaaagaacaatgagaggatcgactccattaaataatttatcatggagtgataatgtcctgtttccatggcatattacaaaatcgattccgtccaagatgataaaaacatttaccggcatcataaacacggagtttattttatatgtctcgcataaacattactaaaaaaatatattgttcggttttctttcacatctttaattatgaaaaagtaaatcattatgagatggacgcatcgttcgcgacagtatgtggtacatacctaacgtatttatggacgacggtaagaatgaaggtcacgtttctgtcaacaatgtcgacgcgatcgtgtaacacgactcacaatagaatctgtgaatgctctcccgatcatggatgcaaggcatgtgtttcccaaacaaaatgtggaataggatacggagtatccggagacgtcatctgttctccgtgtggtctcggaacatattctcacaccgtctcttccgcagataaatgcgaacccgtacccagaaatacctttaactatatcgatgtggaaattaacctgtatccagttaacgacacatcgtgtactcggacgaccactaccggtctcagcgaatccatctcaacgtcggaactaactattactatgaatcataaagactgcgatcccgtcttcttaataaggtagcgacttcaggtttctttacaggagaaaggtgtgcactctgaatttcgagattaaatgcaataacaaagattcttcctccaaacagttaacgaaagcaaagaatgatactatcatgccgcattcggagacagtaactctagcgtcgacatctatatactatatagtaataccaatactcaagactacgaaactgatacaatctcttatcatgtgggtaatgtagccatatgcccggtagttgcgatatacataaactgatcactaattccaaacccacccgctttttatagtaagtttttcacccataaatacaataattaatttctcgtaaaagtagaaaatatattctaatttattgcacggtaaggaagtagaatcataaagaacagtactcaatcaatagcaatcatgaaacaatatatcgtactggcatgcatgtgcctgccagtcttcagcaatcatcctcatcgtgtacggaagaagaaaacaaacatcatatgggaatcgatgttattatcaaagtcacaaagcaagaccaaacaccgaccgatgataagatttgccaatccgtaacggaaattacagagtccgagtcagatccagatcccgaggtggaatcagtcgaggatgtagatcctcctaccacttattactccatcatcggtggaggtctgagaatgaactttggattcaccaaatgtcctcagattaaatccatctcagaatccgctgatggaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaagttacctctcgcgacttcttctttttctgtctcaatagtgtgatacgattatgacactattcctatttcctttcagggtatcacaaaaatattaaacctctttctgatggtctcatacaaaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaa

SEQ ID NO: 2 - DNA, 171918 bp, synthetic construct
  misc_feature 1..171918: note = sMVA-S/N
  source 1..171918: mol_type = other DNA
ttttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatattttatgagaccatcaaagagagaaagagaataaaaatattttttatgagaccatcaaagagagaaagagaataaaaatatttttgtatgagaccatcagaaagaggtttaatatttttgtgataccctgaaaggaaataggaatagtgtcataatcgtatcacactattgagacagaaaaagaagaagtcgcgagaggtaactttttgttttgcaaaccggaatatagtgtccggtacacttttttaattcgtggtgtgcctgaatcgttcgattaaccctactcatccaatttcagatgaatagagttatcgattcagacacacgctttgagttttgttgaatcgatgagtgaagtatcatcggttgcaccttcagatgccgatccgtcgacatacttgacctcaagttcagatgattccttgcacatgtctccgatacgaacgctaaactctagattcttgacacattttgtatcgacgatcgttgaaccgatgatatcttcgtaactcactttcttatgagagatgttagacccgagtactggatgggtcttgatgtcgctgtctttctcttcttcgctacatctgatgtcgatagacacctcacagtctttccatcagcggattctgagatggatttaatctgaggacatttggtgaatccaaagttcattctcagacctccaccgatgatggagtaataagtggtaggaggatctacatcctcgactgattccacctcgggatctggatctgactcggactctgtaatttccgttacggattggcaaatcttatcatcggtcggtgtttggtcttgctttgtgactttgataataacatcgattcccatatgatgtttgttttcttcttccgtacacgatgaggatgattgctgaagactggcaggcacatgcatgccagtacgatatattgtttcatgattgctattgattgagtactgttctttatgattctacttccttaccgtgcaataaattagaatatattttctacttttacgagaaattaattattgtatttatgggtgaaaaacttactataaaaagcgggtgggtttggaattagtgatcagtttatgtatatcgcaactaccgggcatatggctacattacccacatgataagagattgtatcagtttcgtagtcttgagtattggtattactatatagtatatagatgtcgacgctagagttactgtctccgaatgcggcatgatagtatcattctttgctttcgttaactgtttggaggaagaatctttgttattgcatttaatctcgaaattcagagtgcacacctttctcctgtaaagaaacctgaagtcgctaccttattaagaagacgggatcgcagtctttatgattcatagtaatagttagttccgacgttgagatggattcgctgagaccggtagtggtcgtccgagtacacgatgtgtcgttaactggatacaggttaatttccacatcgatatagttaaaggtatttctgggtacgggttcgcatttatctgcggaagagacggtgtgagaatatgttccgagaccacacggagaacagatgacgtctccggatactccgtatcctattccacattttgtttgggaaacacatgccttgcatccatgatcgggagagcattcacagattctattgtgagtcgtgttacacgatcgcgtcgacattgttgacagaaacgtgaccttcattcttaccgtcgtccataaatacgttaggtatgtaccacatactgtcgcgaacgatgcgtccatctcataatgatttactttttcataattaaagatgtgaaagaaaaccgaacaatatatttttttagtaatgtttatgcgagacatataaaataaactccgtgtttatgatgccggtaaatgtttttatcatcttggacggaatcgattttgtaatatgccatggaaacaggacattatcactccatgataaattatttaatggagtcgatcctctcattgttctttgcgtatctcaatctgtggcgtttgcttcgtttaaataatatatcaaacatggagacgcctgatatgtaggcattcttcattctattaatgtctgctctatagcgctttagttccttatgacgaccggcgatatcatacttactttagaaggaaaatcatcatctaggattaaggcgtatctgatacaggcgaataatggttcaggatatagatagcgtatatctctattaaatgcgtcaatcatagtctctagagtgggatggtaactcagtaataaatcaactagcttctctttggtaactgcttttctggatggccgtattgattatcgagcgtgacactcgctccatattccaataaccgctttgcaaattgtatattattgacatcgaccgcgtaatatagtagagttatcgatcatatctatatcatccatgtacttgcttagtatatcaaatacatcttcataacagtgatacccgcaattattaaatctcgataatatcagaccgtacatacatagacggccattgttagatatgtgatttacagccgcgtgtccatattttccacgataaaccttacgacgtttacatcgacgagattattattaacaaagttgttgtccgtcgtcttatccaacatgcattgaatgataggtatacttaccatatcgccgtaatgtaagtagtttatcagtatggcttgtacgatggattcatcctgttgtctaaatctctttagaatgttatcgatgatgtagtggttatattctctggaatcgtacgaagtaatactacgcattacgtcgacaagagtatgacgtctctcaataagaagattaacgatttccatgtctacattatatggggttactctaaatcgcttgtttagataatacgcctctaatatagggctgacgtcgtatactctacacgtgtccacatcctttattaataatctctatatctatggttgagcaagaccagtagtattggatggaaacattgttatcgatcaaacatttaattacatccttggatagagattctctatgagacgatatatagtaatgaagagagttcttacacatatcactgttgtacatacaggtacgaaatacgtaaccggtgctgtaacattctgatttaagaagccatagcaatacttctggtctcggattaggcgtcgttacgtatatatccaccaatccgagaccattgattgcataattcgtattcttggacggacgtatccgtttatccacaattaggtattttagcagacgtaagtcgaaatcatttatattcgacttgagttcgttagaggaattcgaatagctggatatcagtagatgcacaatctgagattttacgtatctatgcttactgtatgctcctagcggagttaatccttcgttgtttctacaaagtctctcgactccgcgagagagtaacagtcgaacaatcttaatgtctgtatcgcatttattggagacgtaacaatgtagcgcattgtttcctcgtctatctatatgttttgataagttgtgacacgtttcaatttctagttttatttttttgtacgtcacatcttcatccagtagacgacatagaatacatgtgcaatccatagctattctggtgctaattattcctcataagatgataaaaagtgtagtgagagagcatgaaggagatttagtatttagcagtgcggatatgatccaagagggtgagatagtcgttctcgttcagaatctttcgcagcataagtagtatgtcgatatacttatcgttgaagactcttccagagacgatagctgattgagtacaaagtccaatgattgcacgaagttcttcggcggttttcatggagtcatttctgatgaaacatttaatgatctaaatttcagtttatgtttgtaccccgtattcatacttaacaaattggtattacataccattaataatgcaagcataaaaaatcgttagtagatgtttctaaatataggttccgtaagcaaagaatataagaatgaagcggtaatgataaaatcaatcgttatctaaaatgatcatactcatttattttattctattatattaacacatacatttttaacagcaacacattcaatattgtattgttatttttatattatttacacaattaacaatatattattagtttatattactgaattaataatataaaattcccaatcttgtcataaacacacactgagaaacagcataaacacaaaatccatcaaaaatgttgataaattatctgatgttgttgttcgctgctatgataatcagatcattcgccgatagtggtaacgctatcgaaacgacatcgccagaaattacaaacgctacaacagatattccagctatcagattatgcggtccagagggagatggatattgtttacacggtgactgtatccacgctagagatatcgacggtatgtattgtagatgctctcatggttatacaggcattagatgtcagcatgtagtattagtagactatcaacgttcagaaaaaccaaacactacaacgtcatatatcccatctcccggtattatgcttgtattagtaggcattattattattacgtgttgtctattatctgtttataggttcactcgacgaactaaactacctatacaagatatggttgtgccataatttttataaattttttttatgagtatttttacaaaaatgtataaagtgtatgtcttatgtatatttataaaaatgctaaatatgcgatgtatctatgttatttgtatttatctaaacaatacctctacctctagatattatacaaaaattttttatttcggcatattaaagtaaaatctagttaccttgaaaatgaatacagtgggtggttccgtatcaccagtaagaacataatagtcgaatacagtatccgattgagattttgcatacaatactagtctagaaagaaatttgtaatcatcttctgtgacgggagtccatatatctgtatcatcgtcccatgctatattcctgttatcatcattagttaatgaaaataactctcgtgcttcagaaaagtcaaatattgtatccatacatacatctccaaaactatcgcttatacgtttatctttaacgatacctatacctagatggttatttactaacagacattttccagatctattgactataactcctatagtttccacatcaaccaagtaatgatcatctattgttatataacaataacataactcttttccgtttttatcagtatgtatatctatatcaacgtcgtcgttgtagtgaatagtagtcattgatctattatatgaaacggatatgtctagaacggcaattgttttacgtccagttaacactttcgttgatttaaagtctagagtctttgcaaacataatatccttatccgactttatatttcctgtagggtggtataattttattttgcctccacatatcggtgtttccaaatatattactagacaatattccatatagttattagttaagggtacccaattagaacacgtacgcttattatcatcatttggatcgtatttcataaaagttattgtactatcgatgtcaacacattctacattttttaatcgtctatatagtatttttctgatattttctataatatcagaattgtcttccatcggaagttgtatactatcggaatcagttacatgtttaaataattctctgatgtcattccttatacaatcaaattcattattaaacagtttaatagtctgtagacctttatcgtcgtaaatatccattgtcttattagttacgcttatttttatgtgtttttacgttgctttattatattttataagaatgattgtttgacgaatcacgagaactattaagacacattattaggtatatattataaaaaagtttttgattacgatgttataagaggaaagaggacacattaacatcatacatcaattaactacattcttataacatcgtaatcaaaagaattgcaattttgatgtataacaactgtcaatgggttatggaattgtatattacatattatacggtatgttggtaacgacaaataccgatcggtaattgtctgccggtgtaatagaattatatatatctatctattacaccggccttgtatacataataataagttgtggtagtatgatctccatatttataatttaggactttgtattcagtttttttggaatcataaaaaataaaaaaaagttttactaatttaaaattatttacatttttttcactgtttagtcgcggatatggaattcgatcctgccaaaatcaatacatcatctatagatcatgtaacaatattacaatacatagatgaaccaaatgatataagactaacagtatgcattatcacaaaaataaatccacatttggctaatcaatttcgggcttggaaaaaacgtatcgccggaagggactatatgactaacttatctagagatacaggaatacaacaatcaaaacttactgaaactgtcaaaaaaatagaaacatatatggtctatatatacactacaatttagttattaattggataaccgatgtgattatcaatcaatattaagaaggttggtaaattggtacatagctaataatacctatacacccaataatacaacaaccatttctgagttggatatcatcaaaatactggataaatacgaggacgtgtatagagtaagtaaagaaaaagaatgtgaaatttgctatgaagttgtttactcaaaacgatagatactttggtttattggattcgtgtaatcatatattttgcataacatgcatcaatatatggcatagaacacgaagagaaaccggtgcgtcggataattgtcctatatgtcgtacccgttttagaaacataacaatgagcaagttaactaataaataaaaagtttaatttgttgacgacgtatgtcgttattttttctcgtataaaagattaatttgattctaatataatctttagtattggataaatatcaattcaaattaattccattagattatatcataaataaaaatagtagcacgcactacttcagccaaatattcttttttgaaacgccatctatcgtagtgaggacacaagtgaacctataatgagcaaatttattagtatcggttacatgaaggactttacgtagagtggtgattccactatctgtggtacgaacggtttcatcttctttgatgccatcacccagatgttctataaacttggtatcctttgccaaccaatacatatagctaaactcaggcatatgttccacacatcctgaacaatgaaattctccagaagatgttacaatgtctagatttggacatttggtttcaaccgcgttaacatatgagtgaacacacccatacatgaaagcgatgagaaataggattttcatcttgccaaaatatcactagaaaaaatttatttatcaattttaaaggtataaaaaatacttattgttgctcgaatattttgtatttgatggtatacggaagattagaaatgtaggtattatcatcaactgattctatggttttatgtattctatcatgtttcactattgcgttggaaataatatcatatgcttccacatatattttattttgttttaactcataatactcacgtaattctggattattgacatatctatgaataattttagctccatgatcagtaaatattaatgagaacatagtattaccacctaccattatttttttcatctcattcaattcttaattgcaaagatctatataatcattatagcgttgacttatggactctggaatcttagacgatgtacagtcatctataatcatggcatatttaatacattgttttatagcatagtcgttatctacgatgttagatatttctctcaatgaatcaatcacacaatctaatgtaggtttatgacataatagcattttcagcagttcaatgtttttagattcgttgatggcaatggctatacatgtatatccgttatttgatctaatgttgacatctgaaccggattctagcagtaaagatactagagattgtttattatatctaacagccttgtgaagaagtgtttctcctcgtttgtcaatcatgttaatgtctttaagataaggtaggcaaatgtttatagtactaagaattgggcaagcataagacatgtcacaaagaccctttttgtatgtataagtgtaaaaattataacattcatagttggatttacataggtgtccaatcgggatctctccatcatcgagataattgatggcatctcccttccttttttagtagatatttcatcgtgtaagaatcaatattaatatttctaaagtatccgtgtatagcctctttatttaccacagttccatattccactagagggatatcgccgaatgtcatatactcaattagtatatgttggaggacatccgagttcattgttttcaatatcaaaaagatggtttccttatcatttctccatagtggtacaatactacacattattccgtgcggctttccattttccaaaaacaatttgaccaaatctacatctttattgtatctataatcactatttagataatcagccataattactcgagtgcaacatgttagatcgtctatatatgaataagccgtgttatctattcctttcattaacaatttaacgatgtctatatctatatgagatgacttaatataatattgaagagctgtacaatagtttttatctataaaagacggcttgattccgtgattaattagacatttaacaacttccggacgcacatatgctctcgtatccgactctgaatacagatgagagatgatatacagatgcaatacggtaccgcaatttcgtagttgataatcatcatacgcgtatcagtactcgtcctcataaagaacactgcagccattttctatgaacaaatcaataatttcaggaacaggatcatctgtcattacataattttctataactgaacgatggttttcacatttaacactcaagtcaaatccatgttctaccaacacctttatcaagtcaacgtctacatttttggatttcatatagctgaatatattaaagtcatttatgttgctaaatccagtggcttctagtagagccatcgctatatcctttaactttaacatgtctactatttgtgtattcttctaatggggtagctgtctccaatttttgcgtaatggattagtgccactgtctagtagtagtttgacgacctcgacattattacaatgctcattaaaaaggtatgcgtgtaaagcattattcttgaattggttcctggtatcattaggatctctgtctctcaacatctgtttaagttcatcgagagccacctcctcattttccaaatagtcaaacattttgactgaatgagctactgtgaactctatacacccacacaactaatgtcattaaatatcatgtcaaaaacttgtacaattattaataaaaataatttagtgtttaaattttaccagttccagattttacacctccgttaaccccactttttacaccactggacgatcctcctccccacattccaccgccaccagatgtataagttttagatcctttattactaccatcatgtccatggataaagacactccacatgccgccactaccccctttagaagacatattaataagacttaaggacaagtttaacaataaaattaatcacgagtaccctactaccaacctacactattatatgattatagtttctatttttacagtaccttaactaaagtctctagtcacaagagcaatactaccaacctacactattatatgattatagtttctatttttataggaacgcgtacgagaaaatcaaatgtctaatttctaacggtagtgttgataaacgattatcgtcaatggatacctcctctatcatgtcgtctattttcttactttgttctattaacttattagcattatatattatttgattataaaacttatattgcttattagcccaatctgtaaatatcggattattaacatatcgtttctttgtaggtttatttaacatgtacatcactgtaagcatgtccgtaccatttattttaatttgacgcatatccgcaatttctttttcgcagtcggttataaattctatatatgatggatacatgctacatgtgtacttataatcgactaatatgaagtacttgatacatattttcagtaacgatttattattaccacctatgaataagtacctgtgatcgtctaggtaatcaactgttttcttaatacattcgatggttggtaatttactcagaataatttccaatatcttaatatataattctgctatttctgggatatatttatctgccagtataacacaaatagtaatacatgtaaacccatattttgttattatattaatgtctgcgccattatctattaaccattctactaggctgacactatgcgacttaatacaatgataaagtatactacatccatgtttatatcatcaatatacggcttacaaagttttagtatcgataacacatccaactcacgcatagagaaggtagggaataatggcataatatttattaggttatcatcattgtcattatctacaactaagtttccattttttaaaatatactcgacaactttaggatctctattgccaaatttttgaaaatatttatttatatgcttaaatctatataatgtagctccttcatcaatcatacatttaataacattgatgtatactgtatgataagatacatattctaacaatagatcttgtatagaatctgtatatcttttaagaattgtggatattattacgtaaactattacacaattctaaaatataaaacgtatcacggtcgaataatagttgatcaactatataattatcgattttgtgatttttcttcctaaactgtttacgtaaatagttagatagaatattcattagttcatgaccactatagttactatcgaataacgcgtcaaatatttcccgtttaatatcgcatttgtcaagataataatagagtgtggtatgttcacgataagtataataacgcatctctttttcgtgtgaaattaaatagtttattacgtccaaagatgtagcataaccatcttgtgacctagtaataatataataatagagaactgttttacccattctatcatcataatcagtggtgtagtcgtaatcgtaattgtctaattcatcatcccaattataatattcaccagcacgtctaatctgttctattttgatcttgtatccatactgtatgttgctacatgtaggtattcctttatccaataatagtttaaacacatctacattgggatttgatgttgtagcgtatttttctacaatattaataccatttttgatactatttatttctatacctttcgaaattagtaatttcaataagtctatatcgatgttatcagaacatagatattcgagtatatcaaaatcattgatatttttatagtcgactgacgacaataacaaaatcacaacatcgtttttgatattattatttttcttggtaacgtatgcctttaatggagtttcaccatcatactcatataatggatttgcaccactttctatcaatgattgtgcactgctggcatcgatgttaaatgttttacaactatcatagagtatcttatcgttaaccatgattggttgttgatgctatcgcattttttggtttctttcatttcagttatgtatggatttagcacgtttgggaagcatgagctcatatgatttcagtactgtagtgtcagtactattagtttcaataagatcaatctctagatctatagaatcaaaacacgataggtcagaagataatgaatatctgtaggcttcttgttgtactgtaacttctcgttttgttagatgtttgcatcgtgctttaacatcaatggtacaaattttatcctcgctttgtgtatcatattcgtccctactataaaattgtatattcagattatcatgagatgtgtatacgctaacggtatcaataaacggagcacaccatttagtcataaccgtaatccaaaaatttttaaagtatatcttaacgaaagaagttgtgtcattgtctacggtgtatggtactagatcctcataagtgtatatatctagagtaatgtttaatttatcaaatggttgataatatggatcctcatgacaatttccgaagatggaaatgagatatagacatgcaataaatctaattgcggacatggttactccttaaaaaaatacgaataatcaccttggctatttagtaagtgtcatttaacactatactcatattaatccatggactcataatctctatacgggattaacggatgttctatatacggggatgagtagttttcttctttaactttatactttttactaatcatatttagactgatgtatgggtaatagtgtttaaagagttcgttctcatcatcagaataaatcaatatctctgtttttttgttatacagatgtattacagcctcatatattacgtaatagaacgtgtcatctaccttattaactttcaccgcatagttgtttgcaaatacggttaatcctttgacctcgtcgatttccgaccaatctgggcgtataatgaatctaaactttaatttcttgtaatcattcgaaataatttttagtttgcatccgtagttatcccctttatgtaactgtaaatttctcaacgcgatatctccattaataatgatgtcgaattcgtgctgtatacccatactgaatggatgaacgaataccgacggcgttaatagtaatttactttttcatctttacatattgggtactagttttactatcataagtttataaattccacaagctactatggaataagccaaccatcttagtataacacacatgtcttaaagtttattaattaattacatgttgttttatatatcgctacgaatttaaacagagaaatcagtttaggaaaaaaaaatatctatctacatcatcacgtctctgtattctacgatagagtgctactttaagatgagacatatccgtgtcatcaaaaatatactccattaaaatgattattccggcagcgaacttgatattggatatatcacaacctttgttaatatctacgacaatagacagcagtcccatggttccataaacagtgagtttatctttctttgaagagatattttgtagagatcttataaaactgtcgaatgacatcgcatttatatctttagctaaatcgtatatgttaccatcgtaatatctaaccgcgtctatcttaaacgtttccatcgctttaaagacgtttccgatagatggtctcatttcatcagtcatactgagccaacaaatataatcgtgtataacatctttgatagaatcagactctaaagaaaacgaatcggctttattatacgcattcatgataaacttaatgaaaaatgtttttcgttgtttaagttggatgaatagtatgtcttaataattgttattatttcattaattaatatttagtaacgagtacactctataaaaacgagaatgacataactagttatcaaagtgtctaggacgcgtaattttcatatggtatagatcctgtaagcattgtctgtattctggagctattttctctatatctaatttctgaacgttcaccaatgtctctagccactttggcactaatagcgatcattcgcttagcgtcttctatattattaactggttgattcaatctatctagcaatggaccgtcggacagcgtcattctcatgttcttaatcaatgtacatacatcgccgtcatctaccaattcatccaacaacataagctttttaaaatcatcattataataggtttgatcgttgtcatttctccaaagaatatatctaataagtagagtcctcatgattagttaacaactattttttatgttaaatcaattagtacaccgctatgtttaatacttattcatattttagtttttaggattgagaatcaatacaaaaattaatgcatcattaattttagaaatacttagtttccacgtagtcaatgaaacatttgaactcatcgtacaggacgttctcgtacaggacgtaactataaaccggtttatatttgttcaagatagatacaaatccgataactttttttacgaattctacgggatccactttaaaagtgtcataccgggttctttttatttttttaaacagattaatggtgtgatgttgattaggtcttttacgaatttgatatagaatagcgtttacatattctccataatggtcaatcgccatttgttcgtatgtcataaattctttaattatatgacactgtgtattatttagttcatccttgttcatcattaggaatctatccaatatggcaattatactagaactataggtgcgttgtatacacatattgatgtgtctgtttatacaatccatgctactaccttcgggtaaaattgtagcatcatataccatttctagtactttaggttcattgttatccattgcagaggacgtcatgaacgcatcctaaaaaaatatattatttttatgttattttgttaaaaataatcatcgaatacgaatcatccagtccactgaatagcaaaatctttactattttggtatcttccaatgtggctgcctgatgtaatggaaattcattctctagaagatttttcaatgctccagcgttcaacaacgtacatactagacgcacgttattatcagctattgcataatacaaggcactatgtccatggacatccgccttaaatgcatctttgctagagagaaagcttttcagctgcttagacttccaagtattaattcgtgacagatccatgtctgaaacgagacgctaattagtgtataatttttgtcatattgcaccagaattaataatatctctaatagatctgattagtagatacatggctatcgcaaaacaacatatacacatttaataaaaataatatttattaagaaaattcagatttcacgtacccatcaatataaataaaataatgattccttacaccgtacccatattaaggagattctaccttacccataaacaatataaatccagtaatatcatgtctgatgatgaacacaaatggtgtattaaattccagtttttcaggagatgatctcgccgtagctaccataatagtagatgcctctgctacagttccttgttcgtcgacatctatctttgcattctgaaacattttataaatatataatgggtccctagtcatatgtttaaacgacgcattatctggattaaacatactaggagccatcatttcggctatcgacttaatatccctcttattttcgatagaaaatttagggagtttaagattgtacactttattccctaattgaaacgaccaatagtctaattttgcagccgtaatagaatctgtgaaatgggtcatattatcacctattgccaggtacatactaatattagcatccttatacggaaggcgtaccatgtcatattctttgtcatcgattgtgattgtatttccttgcaatttagtaactacgttcatcatgggaaccgttttcgtaccgtacttattagtaaaactagcattgcgtgttttagtgatatcaaacggatattgccatatacctttaaaatatatagtattaatgattgcccatagagtattattgtcgagcatattagaatctactacattagacataccggatctacgttctactatagaattaattttattaaccgcatctcgtctaaagtttaatctatataggccgaatctatgatattgttgataatacgacggtttaatacacacagtattatctacgaaactttgataagttagatcagtgtacgtatatttagatgttttcagcttagctaatcctgatattaattctgtaaatgctggacccagatctctttttctcaaatccatagtcttcaataattctattctagtattacctgatgcaggcaatagcgacataaacatagaaaacgaataaccaaacggtgagaagacaatattatcatcttgaatatttttatacgctactataccggcattggtaaatccttgtagacgataggtagacgctgaacacgttaacgatagtatcaataacgcaatcatgattttatggtattaataattaaccttatttttatgttcggtataaaaattattgatgtctacacatccttttgtaattgacatctatatatccttttgtataatcaactctaatcactttaacttttacagttttccctaccagtttatccctatattcaacatatctatccatatgcatcttaacactctctgccaagatagcttcagagtgaggatagtcaaaaagataaatatatagagcataatcattctcgtatactctgccctttattacatcgcccgcattgggcaacgaataacaaaatgcaagcatcttgttaacgggctcgtaaattgggataaaaattatgtttttatatctattttattcaagagaatattcaggaatttctttttccggttgtatctcatcgcagtatatatcatttgtacattgtttcatattttttaatagtttacaccttttagtaggactagtatcgtacaattcatagctgtattttgaattccaatcacgcataaaaatatcttctaattgttgacgaagacctaatccatcatccggtgtaatattaatagatgctccacatgtatccgtaaagtaatttcctgtccaatttgaggtacctatataggccgttttatcggttaccatatatttggcatggtttaccctagaatacggaatgggaggatcagcatctggtacaataaatagctttacttctatatttatgtttttagattttagcatagcgatagatcttaaaaagtttctcatgataaacgaagatcgttgccagcaactaatcaatagcttaacggatacttgtctgtctatagcggatcttcttaattcatcttctatataaggccaaaacaaaattttacccgccttcgaataaataatagggataaagttcataacagatacataaacgaatttactcgcatttctaatacatgacaataaagcggttaaatcattggttctttccatagtacatagttgttgcggtgcagaagcaataaatacagagtgtggaacaccacttacgttaatactaagaggatgatctgtattataatacgacggataaaagtttttccaattatatggtagattgttaactccaagataccagtatacctcaaaaatttgagtgagatccgctgccaagttcctattattgaagatcgcaatacccaattctttgacctgagttagtgatctccaatccatgttagcgcttcctaaataaatatgtgtattatcagatatccaaaattttgtatgaagaactcctcctaggatatttgtaatatctatgtatcgtacttcaactccggccatttgtagtctttcaacatcctttaatggtttgttagatttattgacggctactctaactcgtactcctcttttgggtaattgtacaatctcgtttaatattatcgtgccgaaattcgtacccacttcatccgataaactccaataaaaagatgatatatctagtgtttttgtggtattggatagaatttccctccacatgttaaatgtagacaaatatactttatcaaattgcatacctataggaatagtctctgtaatcactgcgattgtattatccggattcattttatttgttaaaagaataatcctatatcacttcactctattaaaaatccaagtttctatttctttcatgactgattttttaacttcatccgtttccttatgaagatgatgtttggcaccttcataaatttttatttctctattacaatttgcatgttgcatgaaataatatgcacctaaaacatcgctaatctcattgtttgttccctggagtatgagagtcggggtgttaatcttggaaattatttttctaaccttgttggtagccttcaagacctgactagcaaatccagccttaattttttcatgattgattaatgggtcgtattggtatttataaactttatccatatctctagatactgattctggacatagctttccgactggcgcatttggtgtgatggttcccataagtttggcagctagcagattcagtcttgaaacagcatctgcattaactagaggagacattagaatcattgctgtaaacaagtttggattatcgtaagaggctagtatagaaattgttgctcccatggaatgacccaataagtagatttaatagttaccacgtgctgtaccaaagtcatcaatcatcattttttcaccattacttcttccatgtccaatatgatcatgtgagaatactaaaattcctaacgatgatatgttttcagctagttcgtcataacgtccagaatgtttaccagctccatgacttatgaatactaatgccttaggatatgtaatcattgtccagattgaacatacagtttgcactcatgattcacgttatataactatcaatattaacagttcgtttgatgatcatattatttttatgttttattgataattgtaaaaacatacaattaaatcaatatagaggaaggagacggctactgtcttttgtgagatagtcatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattgatgatataggggtcttcataacgcataattattacgttagcattctatatccgtgttaaaaaaaattatcctatcatgtatttgagagttttatatgtagcaaacatgatagctgtgatgccaataagctttagatattcacgcgtgctagtgttagggatggtattatctggtggtgaaatgtccgttatataatctacaaaacaatcatcgcatatagtatgcgatagtagagtaaacatttttatagtttttactggattcatacatcgtctacccaattcggttatgaatgaaattgtcgccaatcttacacccaaccccttgttatccattagtatagtattaacttcgttatttatgtcataaactgtaaatgattttgtagatgccatatcatacatgatattcatgtccctattataatcattactaactttatcacaatatatgttgataatatctatatatgatctagtctttgtgggcaactgtctatacaagtcgtctaaacgttgtttactcatatagtatcgaacagccatcattacatggtcccgttccgttgatagataatcgagtatgttagtggacttgtcaaatctatataccatattttctggaagtggatatacatagtcgtgatcaacattattgctagcctcatcttctatatcctgtactataccatctacataatctacgatattattacacataaacatcgacaacatactattgtttattatctaagtcctgttgatccaaacccttgatctcctctatttgtactatctagagattgtacttcttccagttctggataatatatacgttgatagattagctgagctattctatctccagtatttacattaaacgtacattttccattattaataagaatgactcctatgtttcccctataatcttcgtctattacaccacctcctatatcaatgccttttagtgacagaccagacctaggagctattctaccatagcaaatcttaggcatggacatactaatatctgtcttaattaactgtctttctcctggagggatagtataatcgtaagcgctatacaaatcatatccggcagcacccggcgattgcctagtaggagatttagctctgttagtttccttaacaaatctaactggtgagttaatattcatgttgaacataaaactaatattttatttcaaaattatttaccatcccatatattccatgaataagtgtgatgattgtacacttctatagtatctatatacgattcacgataaaatcctcctatcaatagcagtttattatccactatgatcaattctggattatccctcggataaataggatcatctatcagagtccatgtattgctggattcacaataaaattccgcatttctaccaaccaagaataaccttctaccgaacactaacgcgcatgatttataatgaggataataagtggatggtccaaactgccactgatcatgattgggtagcaaatattctgtagttgtatcagtttcagaatgtcctcccattacgtatataacattgtttatggatgccactgctggattacatctaggtttcagaagactcggcatattaacccaagcagcatccccgtggaaccaacgctcaacagatgtgggatttggtagacctcctactacgtataatttattgttagcgggtatcccgctagcatacagtctggggctattcatcggaggaattggaatccaattgtttgatatataatttacagctatagcattgttatgtatttcattgttcatccatccaccgatgagatatactacttctccaacatgagtacttgtacacatatggaatatatctataatttgatccatgttcataggatactctatgaatggatacttgtatgatttgcgtggttgtttatcacaatgaaatattttggtacagtctagtatccattttacattatttatacctctgggagaaagataatttgacctgattacatttttgataaggagtagcagatttcctaatttatttcttcgctttatataccacttaatgacaaaatcctcatctggaacatttagttcatcgctttctagaataagtttcatagatagataatcaaaattgtctatgatgtcatcttccagttccaaaaagtgtttggcaataaagtttttagtatgacataagagattggatagtccgtattctatacccatcatgtaacactcgacacaatattcctttctaaaatctcgtaggataaagtttatacaagtgtagatgataaattctacagaggttaatatagaagcacgtaataaattgacgacgttatgactatctatatatacctttccagtatatgagtaaataactatagaagttagactgtgaatgtcaaggtctagacaaaccctcgtaactggatctttatttttcgtgtatttttgacgtaaatgtgtgcgaaagtaaggagataactttttcaatatcgtagaattgactattatattgcctcctatggcatcaataattgttttgaatttcttagtcatagacaatgctaatatattcttacagtacacagtattgacaaatatcggcatttatgtttctttaaaagtcaacatctaaagaaaaatgattatcttcttgagacataactcccattttttggtattcacccacacgtttttcgaaaaaattagttttaccttctaatgatatattttccatgaaatcaaacggattggtaacattataaatttttttaaatcccaattcagaaatcaatctatccgcgacgaattctatatatgttttcatcatttcacaattcattcctataagtttaactggaagagccgcagtaagaaattcttgttcaatggataccgcatctgttataatagatctaacggtttcttcactcggtggatgcaataaatgtttaaacatcaaacatgcgaagtcgcagtgtagaccctcgtctctactaatcaattcgttggaaaacgtgagtccgggcattaggccacgctttttaagccaaaatatggaagcgaatgatccggaaaagaagattccttctactgcagcaaaggcaataagtctctctccataaccggcgctgtcatgtatccacttttgagcccaatcggccttcttttttacacaaggcatcgtttctatggcattaaagagatagtttttttcattactatctttaacataagtatcgatcaaaagactatacatttccgaatgaatgttttcaatggccatctgaaatccgtagaaacatctagcctcggtaatctgtacttctgtacaaaatcgttccgccaaattttcattcactattccgtcactggctgcaaaaaacgccaatacatgttttataaaatatttttcgtctggtgttagtttattccaatcattgatatctttagatatatctacttcttccactgtccaaaatgatgcctctgcctttttatacatgttccagatgtcatgatattggattgggaaaataacaaatctatttggatttggtgcaaggatgggttccataactaaattaacaataacaataaattttttttcagttatctatatgcctgtacttggatcttttgtacatcgatatcgccgcaatcactacaataattacaagtattattgatagcattgttattagtactatcataattaaattatcgttattatcattttgtaattgtgacatcatactagataaatcgtttgcgagattgttgtgggaagcgggcatggaggatgaattatcgttattattatttaaagcctcccattcggattcacaaatatggcgcgcgttcaacattttatggaaacagataacaagaaaactcgtcatcgttcaaatttttaacgatagtaaaccgattaaacgtcgagctaatttctaacgctagcgactctgttggatatgggtttccagatatatatcttttcagttcccctacgtatctataatcatctgtaggaaatggaagatatttccatttatctactgttcctaatatcatatgtggtggtgtagtagaaccattaagcgcgaaagatgttatttcgcatcgtattttaacttcgcaataatttctggttagataacgcactctaccagtcaagtcaatgatattagcctttacagatatattcatagtagtcgtaacgatgactccatcttttagatgcgatactcctttgtatgtaccagaatcttcgtacctcaaactcgatatatttaaacaagttaatgagatattaacgcgttttatgaatgatgatatataaccagaagttttatcctcggtggctagcgctataaccttatcattataataccaactagtgtgattaatatgtgacacgttagtgtgggtacaaatatgtacattatcgtctacgtcgtattcgatacatccgcatacagccaacaaatataaaatgacaaatactctaacgccgttcgtacccatcttgatgcggtttaataaatgttttgatttcaatttattgtaaaaaaagattcggttttatactgttcgatattctcattgcttatattttcatctatcatctccacacagtcaaatccgtggttagcatgcacctcatcaaccggtaaaagactatcggactcttctatcattataactctagaatatttaatttggtcattattaatcaagtcaattatcttatttttaacaaacgtgagtattttactcattttttataaaaacttttagaaatatacagactctatcgtgtgtctatatcttctttttatatccaatgtatttatgtctgatttttcttcatttatcatatataatggtccaaattctacacgtgcttcggattcatccagatcattaaggttcttataattgtaacatccttctcttccctcttctacatcttccttcttattcttattcttagcgtcacagaatctaccacagcaggatcccatgacgagcgtcatattaaactaatccattttcaattataatatacgattagtaatgaccattaaaataaaaatattcttcataaccggcaagaaagtgaaaagttcacattgaaactatgtcagtagtatacatcatgaaatgatgatatatatatactctattttggtggaggattatatgatataattcgtggataatcattcttaagacacatttcttcattcgtaaatcttttcacgttaaatgagtgtccatattttgcaatttcttcatatgatggcggtgtacgtggacgaggctgctcctgttcttgttgtagtcgccgactgtcgtgtctgcgtttagatccctccattatcgcgattgcgtagatggagtactattttataccttgtaattaaatttttttattaattaaacgtataaaaacgttccgtatctgtatttaagagccagatttcgtctaatagaacaaatagctacagtaaaaataactagaataattgctacacccactagaaaccacggatcgtaatacggcaatcggttttcgataataggtggaacgtatattttatttaaggacttaacaattgtctgtaaaccacaatttgcttccgcggatcctgtattaactatctgtaaaagcatatgttgaccgggcggagccgaacattctccgatatccaatttctgtatatctataatattattaacctccgcatacgcattacagttcttttctagcttggataccgcactaggtacatcgtctagatctattcctatttcctcagcgatagctcttctatccttttccggaagcaatgaaatcacttcaataaatgattcaaccatgagtgtgaaactaagtcgagaattactcatgcatttgttagttattcggagcgcgcaatttttaaactgtcctataacctctcctatatgaatagcacaagtgacattagtagggatagaatgttgagctaatttttgtaaataactatctataaaaagattatacaaagttttaaactctttagtttccgccatttatccagtctgagaaaatgtctctcataataaatttttccaagaaactaattgggtgaagaatggaaacctttaatctatatttatcacagtctgttttggtacacatgatgaattcttccaatgccgtactaaattcgatatctttttcgatttctggatatgtttttaataaagtatgaacaaagaaatggaaatcgtaataccagttatgtttaactttgaaattgttttttattttcttgttaatgattccagccacttgggaaaagtcaaagtcgtttaatgccgatttaatacgttcattaaaaacaaactttttatcctttagatgaattattattggttcattggaatcaaaaagtaagatattatcgggtttaagatctgcgtgtaaaaagttgtcgcagcatggtagttcgtaaattttaatgtataacagagccatctgtaaaaagataaactttatgtattgtaccaaagatttaaatcctaatttgatagctagctcggtatctactttatctgccgaatacagtgctaggggaaaaattataatatttcctctttcgtattcgtagttagttctcttttcatgttcgaaaaagtgaaacatgcggttaaaatagtttataacattaatattactgttaataactgccggataaaagtgggatagtaatttcacgaatttgatactgtcctttctctcgttaaacgcctttaaaaaaactttagaagaatatctcaatgatagttcctgaccatccatagtttgtatcaataatagcaacatatgaagaacacgtttatacagagtatgtaaaaatgttaatttatagtttaatcccatggcccacgcacacacgattaattttttttcatctccctttagattgttgtatagaaatttgggtactgtgaactccgccgtagtttccatgggactatataattttgtggcctcgaatacaaattttactacatagttatctatcttaaagactataccatatcctcctgtagatatgtgataaaaatcgtcgtttataggataaaatcgtttatccttttgttggaaaaaggatgaattaatgtaatcattctcttctatctttagtagtgtttccttattaaaattcttaaaataatttaacaatctaactgatggagcccaattttggtgtaaatctaattgggacattatattgttaaaatacaaacagtctcctaatataacagtatctgataatctatggggagacatccattgatattcaggggatgaatcattggcaacacccatttattgtacaaaaagccccaatttacaaacgaaagtccaggtttgatagagacaaactattaactattttgtctctgtttttaatttctttggtaatgaaattattcacaatatcagtatcttctttatctaccagagattttactaacttgataaccttggctgtctcattcaatagggtagtaatatttgtatgtgtgatattgatatcttttagaagtgattctttgatggtgccagcatacgaattacaataatgcagaaactcggttaacatgcaggaattatagtaagccaattccaattgttgcctgtgttgtattagagtgtcaatatgagcaatggtgtccttgcgtttctctgatagaatgcgagcagcgattttggcgttatcatttgacgatatttctggaatgacgaatcctgtttctactaactttttggtaggacaaagtgaaacaatcaagaagatagcttctcctcctatttgtggaagaaattgaactcctctagatgatctccttgacagatattggaccgaattacagaagtacctggaatgtaaagccctgaaaccccctcattttttaagcagattgttgccgtaaatcctgcactatgcccaagatagagagctcctttggtgaatccatctctatgtttcagtttaaccaagaaacagtcagctggtctaaaatttccatctctatctaatacagcatctaacttgatgtcaggaactatgaccggttatgttatatgtaacattgagtaaatccttaagttcataatcatcactgtcatcagttatgtacgatccaaacaatgtttctactggcatagtggatacgaagatgctatccatcagaatgtttccctgattagtattttctatatagctattcttctttaaacgattttccaaatcagtaactatgttcatttttttaggagtaggacgcctagccagtatggaagaggattttctagatcctctcttcaacatctttgatctcaatggaatgcaaaaccccatagtgtaacaaccaacgataaaaataatattgtttttcactttttataattttaccatctgactcatggattcattaatatctttataagagctactaacgtataattctttataactgaactgagatatatacaccggatctatggtttccataattgagtaaatgaatgctcggcaataactaatggcaaatgtataaaacaacgaaattatactagagttgttaaagttaatattttctatgagctgttccaataaattatttgttgtaactgcgttcaagtcataaatcatcttgatactatccagtaaaccgtttttaagttctggaatattattatcccattgtaaagcccctaattcgactatcgaatatcctgctctgatagcagtttcaatatcgacggacgtcaatactgtaataaaggtggtagtattgtcatcatcgtgataaactactggaatatggtcgttagtaggtacggtaactttacacaacgcgatatataactttccttttgtaccatttttaacgtagttgggacgtcctgcagggtattgttttgaagaaatgatatcgagaacagatttgatacgatatttgttggattcctgattatttactataatataatctagacagatagatgattcgataaatagagaaggtatatcgttggtaggataatacatccccattccagtattctcggatactctattaatgacactagttaagaacatgtcttctattctagaaaacgaaaacatcctacatggactcattaaaacttctaacgctcctgattgtgtctcgaatgcctcgtacaaggatttcaaggatgccatagattctttgaccaacgatttagaattgcgtttagcatctgatttttttattaaatcgaatggtcggctctctggtttgctaccccaatgataacaatagtcttgtaaagataaaccgcaagaaaatttatacgcatccatccaaataaccctagcaccatcggatgatattaatgtattattatagattttccatccacagttattgggccagtatactgttagcaacggtatatcgaatagattactcatgtaacctactagaatgatagttcgtgtactagtcataatatctttaatccaatctaagaaatttaaaattagattttttacactgttaaagttaacaaaggtattacccggatacgtggatatcatatatggtattggtccattatcagtaatagctccataaactgatacggcgatggtttttatatgtgtttgatctaacgaggaagaaattcgcgcccacaattcatctctagatatgtatttaatatcaaacggtaacacatcaatttcgggacgcgtatatgtttctaaatttttaatccaaatataatgatgacctatatgccctattatcatactgtcaactatagtacacctagagaacttacgatacatctgtttcctataatcgttaaattttacaaatctataacatgctaaaccttttgacgacaaccattcattaatttctgatatggaatctgtattctcaataccgtatcgttctaaagccagtgctatatctccctgttcgtgagaacgctttcgtataatatcgatcaacggataatctgaagtttttggagaataatatgactcatgatctatttcgtccataaacaatctagacataggaattggaggcgatgatcttaattttgtgcaatgagtcgtcaatcctataacttctaatattgtaatattcatcatcgacataacactatctatgttatcatcgtatattagtataccatgaccttcttcatttcgtgccaaaatgatatacagtcttaaatagttacgcaatatctcaatagtttcataattgttagctgttttcatcaaggtttgtatcctgtttaacatgatggcgttctatacgtttctattttttaaatttttaacgatttactgtggctagatacccaatctctctcaaatatttttttagcctcgcttacaagctgtttatctatactattaaaactgacgaatccgtgattttggtaatgggttccgtcgaaatttgccgaagtgatatgaacatattcgtcgtcgactatcaacaattttgtattattctgaatagtgaaaaccttcacagatagatcattttgaacacacaacgcgtctagacttctggcggttgccatagaatatacgtcgttcttatcccaattaccaactagaagtctgatcttaactcctctattaatggctgcttctataatggagttgtaaatgtcgggccaatagtagctattaccgtcgacacgtgtagtgggaactatggccaaatgttcaatatctatactagtcttagccgacttgagtttatcaataactacatcagtgtctagatctctagaatatcccaataggtgttccggagaatcagtaaagaacactccacctataggattcttaatatgatacgcagtgctaactggcagacaacaagccgcagagcataaattcaaccatgaattttttgcgctattaaaggctttaaaagtatcaaatcttctacgaagatctgtggccagcgggggataatcagaatatacacctaacgttttaatcgtatgtatagatcctccagtaaatgacgcgtttcctacataacatctttcattatctgacacccaaaaacaaccgagtagtagtcccacattattttttttatctatattaacggttataaaatttatatccgggcagtgactttgtagctctcccagatttcttttccctcgttcatctagcaaaactattattttaatccctttttcagatgcctcttttagtttatcaaaaataagcgctcccctagtcgtactcagaggattacaacaaaaagatgctatgtatatatatttcttagctagagtgataatttcgttaaaacattcaaatgttgttaaatgatcggatctaaaatccatattttctggtagtgtttctaccagcctacattttgctcccgcaggtaccggtgcaaatggccacatttagttaacataaaaacttatacatcctgttctatcaacgattctagaatatcatcggctatatcgctaaaattttcatcaaagtcgacatcacaacctaactcagtcaatatattaagaagttccatgatgtcatcttcgtctatttctatatccgtatccattgtagattgttgaccgattatcgagtttaaatcattactaatactcaatccttcagaatacaatctgtgtttcattgtaaatttataggcggtgtatttaagttggtagattttcaattatgtatcaatatagcaacagtagttcttgctcctccttgattctagcatcctcttcattattttcttctacgtacataaacatgtccaatacgttagacaacacaccgacgatggcggccgccacagacacgaatatgactaaaccgatgaccatttaaaaacccctctctagctttcacttaaactgtatcgattattcttttagaacatgtataatataaaaacattattctatttcgaatttaggcttccaaaaatttttcatccgtaaaccgataataatatatatagacttgttaatagtcggaataaatagattaatgcttaaactatcatcatctccacgattagagatacaatatttacattttttttgctgtttcgaaactttatcaatacacgttaatacaaacccaggaaggagatattgaaactgaggctgttgaaaatgaaacggtgaatacaataattcagataatgtaaaatcatgattccgtattctgatgatattagaactgctaatggatgtcgatggtatgtatctaggagtatctattttaacaaagcatcgatttgctaatatacaattatcattttgattaattgttattttattcatattcttaaaaggtttcatatttatcaattcttctacattaaaaatttccatttttaatttatgtagccccgcaatactcctcattacgtttcattttttgtctataatatccattttgttcatctcggtacatagattatccaattgagaagcgcatttagtagttttgtacattttaagtttattgacgaatcgtcgaaaactagttatagttaacattttattatttgataccctgatattaatacccctgccgttactattatttataactgatgtaatccacgtaacattggaattaactatcgatagtaatgcatcgacgcttccaaaattgtctattataaactcaccgataatttttttattacatgttttcatattcattaggattattaaatctttaatcttactacgattgtatgcgttgatattgcaagacgtcattctaaaagacggaggatctccatcaaatgccagacaatcacgtacaaagtacatggaaataggttttgttctattgcgcatcatagatttatatagaacacccgtagaaatactaatttgttttactctataaaatactaatgcatctatttcatcgttttgtataacgtctttccaagtgtcaaattccaaatttttttcattgatagtaccaaattcttctatctctttaactacttgcatagataggtaattacagtgatgcctacatgccgttttttgaaactgaatagatgcgtctagaagcgatgctacgctagtcacaatcaccactttcatatttagaatatatatatgtaaaaatatagtagaatttcattttgttttttttctatgctataaatgaattctcattttgcatctgctcatactccgttttatatcaataccaaagaaggaagatatctggttctaaaagccgttaaagtatgcgatgttagaactgtagaatgcgaaggaagtaaagcttcctgcgtactcaaagtagataaaccctcatcacccgcgtgtgagagaagaccttcgtccccttccagatgcgagagaatgaataacccaggaaaacaagttccgtttatgaggacggacatgctacaaaatatgttcgcggctaatcgcgataatgtagcttctagacttttgtcctaaaatacaattatatccttttcgatattaataaatccgtgtcgtccaggttttttatctctttcagtatgtgaatagataggtattttatctctattcatcatcgaatttaagagatccgataaacattgtttgtattctccagatgtcagcatctgatacaacaatatatgtgcacataaacctctggcacttatttcatgtaccttccccttatcactaaggagaatagtatttgagaaatatgtatacatgatattatcatgaattagatatacagaatttgtaacactctcgaaatcacacgatgtgtcggcgttaagatctaatatatcactcgataacacattttcatctagatacactagacattttttaaagctaaaatagtctttagtagtaacagtaactatgcgattattttcatcgatgatacatttcatcggcatattattacgcttaccatcaaagactataccatgtgtatatctaacgtattctagcatggttgccatacgcgcattaaacttttcaggatctttggatagatcttccaatctatctatttgagaaaacatttttatcatgttcaatagttgaaacgtcggatccactatatagatattatctataaagattttaggaactacgttcatggtatcctggcgaatattaaaactatcaatgatatgattatcgttttcatcttttatcaccatatagtttctaagatatgggattttacttaatataatattatttcccgtaataaattttattagaaatgccaaatctataagaaaagtcctcgaattagtttgaagaatatctatatcgccgtaccgtatatttggattaattagatatagagaatatgatccgtaacatatacaacttttattatggcgtctaagatattcttccatcaacttattaacatttttgactagggaagatacattatgacgtcccattacttttgccttgtctattactgcgacgttcatagaatttagcatatctcttgccaattcttccattgatgttacattataagaaattttagatgaaattacatttggagctttaatagtaagaactcctaatatgtccgtgtatgtggtcactaatacagattgtagttctataatcgtaaataatttacctatattatatgtttgagtctgtttagaaaagtagctaagtatacgatcttttatttctgatgcagatgtatcaacatcggaaaaaaatctttttttattcttttttactaaagatacaaatatgtctttgttaaaaacagttattttctgaatatttctagcttgtaattttaacatatgatattcgttcacactaggtactctgcctaaataggtttctataatctttaatgtaatattaggaaaagtattctgatcaggattcctattcattttgaggatttaaaactctgattattgtctaatatggtctcaacacaaactttttcacagagcgatagagtttttgataactcgtttttcttaagaaatataaaactactgtctccagagctcgctctatcttttattttatttaattcgatacaaactcctgatactggttcagaaagtaattcattaattttcagtcctttatagaagatatttaatatagataatacaaaatcttcagtttttgatatcgatctgattgatcctagaactagatatattaataacgtgctcattaggcagtttatggcagcttgataattagatatagtatattccagttcatatttattagataccgcattgcccagattttgatattctatgaattcctctgaaaataaatccaaaataactagacattctattttttgtggattagtgtactctcttccctctatcatgttcactactggtgtccacgatgataaatatctagagggaatataatatagtccataggatgccaatctagcaatgtcgaataactgtaattttattcttcgctcttcattatgaattgattcttgaggtataaacctaacacaaattatattattagacttttcgtatgtaatgtctttcatgttataagtttttaatcctggaatagaatctattttaatgaggcttttaaacgcagagttctccaacgagtcaaagcataatactctgttggttttcttatatacgatgttacgattttcttctttgaatggaataggtttttgaattagtttataattacaacataatagataaggaagtgtgcaaatagtacgcggaaaaaacataatagctcccctgttttcatccatggttttaagtaaatgatcactggcttctttagtcaatggatattcgaacattaaccgtttcatcatcattggacagaatccatatttcttaatgtaaagagtgatcaaatcattgtgtttattgtaccatcttgttgtaaatgtgtattcggttatcggatctgctcctttttctattaaagtatcgatgtcgatctcgtctaagaattcaactatatcgacatatttcatttgtatacacataaccattactaacgtagaatgtataggaagagatgtaacgggaacagggtttgttgattcgcaaactattctaatacataattcttctgttaatacgtcttgcacgtaatctattatagatgccaagatatctatataattattttgtaagatgatgttaactatgtgatctatataagtagtgtaataattcatgtatttcgatatatgttccaactctgtctttgtgatgtctagtttcgtaatatctatagcatcctcaaaaaatatattcgcatatattcccaagtcttcagttctatcttctaaaaaatcttcaacgtatggaatataataatctattttacctcttctgatatcattaatgatatagtttttgacactatcttctgtcaattgattcttattcactatatctaagaaacggatagcgtccctaggacgaactactgccattaatatctctattatagcttctggacataattcatctattataccagaattaatgggaactattccgtatctatctaacatagttttaagaaagtcagaatctaagacctgatgttcatatattggttcatacatgaaatgatctctattgatgatagtgactatttcattctctgaaaattggtaactcattctatatatgctttccttgttgatgaaggatagaatatactcaatagaatttgtaccaacaaactgttctcttatgaatcgtatatcatcatctgaaataatcatgtaaggcatacatttaacaattagagacttgtctcctgttatcaatatactattcttgtgataatttatgtgtgaggcaaatttgtccacgttctttaattttgttatagtagatatcaaatccaatggagctacagttcttggcttaaacagatatagtttttctggaacgaattctacaacattattataaaggactttgggtagataagtgggatgaaatcctattttaattaatgcgatagccttgtcctcgtgcagatatccaaacgcttttgtgatagtatggcattcattgtctagaaacgctctacgaatatctgtgacagatatcatctttagagaatatactagtcgcgttaatagtactacaatttgtattttttaatctatctcaataaaaaaattaatatgtatgattcaatgtataactaaactactaactgttattgataactagaatcagaatctaatgatgacgtaaccaagaagtttatctactgccaatttagctgcattatttttagcatctcgtttagattttccatctgccttatcgaatactcttccgtcgatatctacacaggcataaaatgtaggagagttactaggccccactgattcaatacgaaaagaccaatctctcttagttatttggcagtactcattaataatggtgacagggttagcatctttccaatcaataatttttttagccggaataacatcatcaaaagacttatgatcctctctcattgatttttcgcgggatacatcatctattatggcgtcagccataacatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatgtttttaatagccgcacacacaatctctgcgtcagaacgctcgtcaatatagatcttagacatttttagagagaactaacacaaccagcaataaaactaatttattttatcatttttttattcatcatcctctggtggttcgtcgtttctatcgaatgtggatctgattaacccgtcatctataggtgatgctggttctggagattctggaggagatggattattatctggaagaatctctgttatttccttgttttcatgtatcgattgcgttgtaacattaagattgcgaaatgctctaaatttgggaggcttaaagtgttgtttgcaatctctacacgcatgtctaactagtggaggttcgtcagcggctctagtttgaatcatcatcggcgtagtattcctacttttacagttaggacacggtgtattgtatttctcgtcgagaacgttaaaataatcgttgtaactcacatcctttattttatctatattgtattctactcctttcttaatgcattttataccgaataagagatagcgaaggaattctttttcggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggctaccgccattgatgtttgttcattggatgagtaactactaatgtatacattttccatttataacacttatgtattaactttgttcatttatattttttcattattatgttgatattaacaaaagtgaatatatatgttaataattgtattgtggttatacggctacaatttcataatgagtggaagtcagtgtccgatgatcaatgacgatagctttactctgaaaagaaagtatcaaatcgatagtgcggagtcaacaataaaaatggataagaagaggataaagtttcagaatagagccaaaatggtaaaagaaataaatcagacaataagagcagcacaaactcattacgagacattgaaactaggatacataaaatttaagagaatgattaggactactactctagaagatatagcaccatctattccaaataatcagaaaacttataaactattctcggacatttcagccatcggcaaagcatcacagaatccgagtaagatggtatatgctctgctgctttacatgtttcccaatttgtttggagatgatcatagattcattcgttatagaatgcatccaatgagtaaaatcaaacacaagatcttctctcctttcaaacttaatcttattagaatattagtggaagaaagattctataataatgaatgcagatctaataaatggagaataattggaacacaagttgataaaatgttgatagctgaatctgataaatatacaatagatgcaaggtataacctaaaacccatgtatagaatcaagggaaaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaaaaatcttcaagagtgcattgactttgttagtgaataggcatttcatctttctccaatactaattcaaattgttaaattaataatggatagtataaatagttattagtgataaaatagtaaaaataattattagaataagagtgtagtatcatagataactctcttctataaaaatggattttattcgtagaaagtatcttatatacacagtagaaaataatatagattttttaaaggatgatacattaagtaaagtaaacaattttaccctcaatcatgtactagctctcaagtatctagttagcaattttcctcaacacgttattactaaggatgtattagctaataccaatttttttgttttcatacatatggtacgatgttgtaaagtgtacgaagcggttttacgacacgcatttgatgcacccacgttgtacgttaaagcattgactaagaattatttatcgtttagtaacgcaatacaatcgtacaaggaaaccgtgcataaactaacacaagatgaaaaatttttagaggttgccgaatacatggacgaattaggagaacttataggcgtaaattatgacttagttcttaatccattatttcacggaggggaacccatcaaagatatggaaatcatttttttaaaactgtttaagaaaacagacttcaaagttgttaaaaaattaagtgttataagattacttatttgggcttacctaagcaagaaagatacaggcatagagtttgcggataatgatagacaagatatatacactctatttcaacaaactggtagaatagtccatagcaatctaacagaaacgtttagagattatatctttcccggagataagactagctattgggtgtggttaaacgaaagtatagctaatgatgcggatattgttcttaatagacacgccattaccatgtatgataaaattcttagttatatatactctgagataaaacaaggacgcgttaataaaaacatgcttaagttagtttatatctttgagcctgaaaaagatatcagagaacttctgctagaaatcatatatgatattcctggagatatcctatctattattgatgcaaaaaacgacgattggaaaaaatattttattagtttttataaagctaattttattaacggtaatacatttattagtgatagaacgtttaacgaggacttattcagagttgttgttcaaatagatcccgaatatttcgataatgaacgaattatgtctttattctctacgagtgctgcggacattaaacgatttgatgagttagatattaataacagttatatatctaatataatttatgaggtgaacgatatcacattagatacaatggatgatatgaagaagtgtcaaatctttaacgaggatacgtcgtattatgttaaggaatacaatacatacctgtttttgcacgagtcggatcccatggtcatagagaacggaatactaaagaaactgtcatctataaaatccaagagtagacggctgaacttgtttagcaaaaacattttaaaatattatttagacggacaattggctcgtctaggtcttgtgttagatgattataaaggagacttgttagttaaaatgataaaccatcttaagtctgtggaggatgtatccgcattcgttcgattttctacagataaaaaccctagtattcttccatcgctaatcaaaactattttagctagttataatatttccatcatcgtcttatttcaaaggtttttgagagataatctatatcatgtagaagaattcttggataaaagcatccatctaaccaagacggataagaaatatatacttcaattgataagacacggtagatcatagaacagaccaaatatattattaataatttgtatatacatagatataattatcacacatttttgataaatgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcagagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataagtataatcccattctaatactttaacctgatgtattacctgcatcttattagaatattaacctaactaaaagacataacatagttgataaaaagcggtaggatataaatattatggctgccaccgttccgcgttttgacgacgtgtacaaaaatgcacaaagaagaattctagatcaagaaacattttttagtagaggtctaagtagaccgttaatgaaaaacacatatctatttgataattacgcgtatggatggataccagaaactgcaatttggagtagtagatacgcaaacttagatgcaagtgactattatcccatttcgttgggattacttaaaaagttcgagtttctcatgtctctatataaaggtcctattcccgtatatgaagaaaaagtaaatactgaattcattgctaatggatcgttctctggtagatacgtatcatatcttcgaaagttttctgcccttccaacaaacgagtttattagttttttgttactgacttccattccaatctataatatcttgttctggtttaaaaatactcagtttgatattactaaacacacattattcagatacgtctatacagataatgccaaacacctggcgttggctaggtatatgcatcaaacaggagactataagcctttgtttagtcgtctcaaagagaattatatatttaccggtcccgttccaataagtatcaaagatatagatcaccctaatcttagtagagcaagaagtccatccgattatgagacattagctaatattagtactatattgtactttaccaagtatgatccggtattaatgtttttattgttttacgtacctgggtattcaattactacaaaaattactccagccgtagaatatctaatggataaactgaatctaacaaagagcgacgtacaactgttgtaaattattttatgcttcgtaaaatgtaggttttgaaccaaacattctttcaaagaatgagatgcataaaactttattatccaatagattgactatttcggacgtcaatcgtttaaagtaaacttcgtaaaatattctttgatcactgccgagtttaaaacttctatcgataattgtttcatatgttttaatatttacaagttttttggtccatggtacattagccggacaaatatatgcaaaataatatcgttctccaagttctatagtttctggattatttttattatattcagtaaccaaatacatattagggttatctgcggatttataatttgagtgatgcattcgactcaacataaataattctagaggagacgatctactatcaaattcggatcgtaaatctgtttctaaagaacggagaatatctatacatacctgattagaattcatccgtccttcagacaacatctcagacagtctggtcttgtatgtcttaatcatattcttatgaaacttggaaacatctcttctagtttcactagtacctttattaattctctcaggtacagattttgaattcgacgatgctgagtatttcatcgttgtatatttcttcttcgattgcataatcagattcttatataccgcctcaaactctattttaaaattattaaacaatactctattattaatcagtcgttctaactctttcgctatttctatagacttatcgacatcttgactgtctatctctgtaaacacggagtcggtatctccatacacgctacgaaaacgaaatctgtaatctataggcaacgatgttttcacaatcggattaatatctctatcgtccatataaaatggattacttaatggattggcaaaccgtaacataccgttagataactctgctccatttagtaccgattctagatacaagatcattctacgtcctatggatgtgcaactcttagccgaagcgtatgagtatagagcactatttctaaatcccatcagaccatatactgagttggctactatcttgtacgtatattgcatggaatcatagatggccttttcagttgaactggtagcctgttttagcatctttttatatctggctctctctgccaaaaatgttcttaatagtctaggaatggttccttctatcgatctatcgaaaattgctatttcagagatgaggttcggtagtctaggttcacaatgaaccgtaatatatctaggaggtggatatttctgaagcaagagctgattatttatttcttcttccaatctattggtactaacaacgacaccgactaatgtttccggagatagatttccaaagatacacacattaggatacagactgttataatcaaagattaatacattattactaaacattttttgttttggagcaaataccttaccgccttcataaggaaacttttgttttgtttctgatctaactaagatagttttagtttccaacaatagctttaacagtggacccttgatgactgtactcgctctatattcgaataccatggattgaggaagcacatatgttgacgcacccgcgtctgtttttgtttctactccataatactcccacaaatactgacacaaacaagcatcatgaatacagtatctagccatatctaaagctatgtttagattataatccttatacatctgagctaaatcaacgtcatcctttccgaaagataatttatatgtatcattaggtaaagtaggacatgatagtacgactttaaatccattttcccaaatatctttacgaattactttacatataatatcctcatcaacagtcacataattacctgtggttaaaacctttgcaaatgcagcggctttgcctttcgcgtccgtagtatcgtcaccgatgaacgtcatttctctaactcctctatttaatactttacccatgcaactgaacgcgttcttggatatagaatccaatttgtacgaatccaatttttcagatttttgaatgaatgaatatagatcgaaaaatatagttccattattgttattaacgtgaaacgtagtattggccatgccgcctactcccttatgactagactgatttctctcataaatacagagatgtacagcttcctttttgtccggagatctaaagataatcttctctcctgttaataactctagacgattagtaatatatctcagatcaaagttatgtccgttaaaggtaacgacgtagtcgaacgttagttccaacaattgtttagctattcgtaacaaaactatttcagaacatagaactagttctcgttcgtaatccatttccattagtgactgtatcctcaaacatcctctatcgacggcttcttgtatttcctgttccgttaacatctcttcattaatgagcgtaaacaataatcgtttaccacttaaatcgatataacagtaacttgtatgcgagattgggttaataaatacagaaggaaacttcttatcgaagtgacactctatatctagaaataagtacgatcttgggatatcgaatctaggtatttttttagcgaaacagttacgtggatcgtcacaatgataacatccattgttaatctttgtcaaatattgctcgtccaacgagtaacatccgtctggagatatcccgttagaaatataaaaccaactaatattgagaaattcatccatggtggcattttgtatgctgcgtttctttggctcttctatcaaccacatatctgcgacggagcattttctatctttaatatctagattataacttattgtctcgtcaatgtctatagttctcatctttcccaacggcctcgcattaaatggaggaggagacaatgactgatatatttcgtccgtaactacgtaataaaagtaatgaggaaatcgtataaatacggtctcgccatttcgacatctggatttcagatataaaaatctgttttcaccgtgactttcaaaccaattaatgcaccgaacatccatttatagaatttagaaatatattttcatttaaatgaatcccaaacattggggaagagccgtatggaccattatttttatagtactttcgcaagcgggtttagacggcaacatagaagcgtgtaaacgaaaactatatactatagtcagcactcttccatgtcctgcatgtagacggcacgcgactatcgctatagaggacaataatgtcatgtctagcgatgatctgaattatatttattattttttcatcagattatttaacaatttggcatctgatcccaaatacgcgatcgatgtgacaaaggttaaccctttataaacttaacccattataaaacttatgattagtcacgactgaaataaccgcgtgattattttttggtataattctacacggcatggtttctgtaactatgaattcaacccccgttacattagtgaaatctttaacaaacagcaagggttcgtcaaagacataaaactcattgtttacaatcgaaatagaccccctatcacacttaaaataaaaaatatccttatcctttaccaccaaataaaattctgattggtcaatgtgaatgtattcacttaacagttccacaaatttatttattaactccgaggcacatacatcgtcggtattttttatggcaaactttactcttccagcatccgtttctaaaaaaatattaacgagttccatttatatcatccaatattattgaaatgacgttgatggacagatgatacaaataagaaggtacggtacctttgtccaccatctcctccaattcatgctctattttgtcattaactttaatgtatgaaaacagtacgccacatgcttccatgacagtgtgtaacactttggatacaaaatgtttgacattagtataattgtccaagactgtcaatctataatagatagtagctataatatattctatgatggtattgaagaagatgacaatcttggcatattgatcatttaacacagacatggtatcaacagatagcttgaatgaaagagaatcagtaattggaataagcgtcttctcgatagagtgtccgtataccaacatgtctgatattttgatgtattccattaaattatttagttttttctttttattctcgttaaacagcatttctgtcaacggaccccaacatcgttgaccgattaagttttgattgatttttccgtgtaaggcgtatctagtcagatcgtatagcctatccaataatccatcatctgtgcgtagatcacatcgtacactttttaattctctatagaagagcgacagacagcaatttctttattctctacagatgtaagatacttgaagacattcctatgatgatgcagaattttggataacacggtattgatggtatctgttaccataattcctttgatggctgatagtgtcagagcacaagatttccaatctttgttttgatatctatatcagacagcatggtgcgtctgacaacacaaggattaagacggaaagatgaaatgattctctcaacatcttcaatggataccttgctattttttctggcattatctatatgtgcgagaatatcctctagagaatcagtatcctttttgatgatagtggatctcaatgacatgggacgtctaaaccttcttattctatcaccagattgcatggtgatttgtcttctttcttttatcataatgtaatctctaaattcatcggcaaattgtctatatctaaaatcataatatgagatgtttacctctacaaatatctgttcgtccaatgttagagtatctacatcagttttgtattccaaattaaacatggcaacggatttaattttatattcctctattaagtcctcgtcgataataacagaatgtagataatcatttaatccatcgtacatggttggaagatgcttgttgacaaaatctttaattgtcttgatgaaggtgggactatatctaacatcttgattaataaaatttataacattgtccataggatactttgtaactagttttatacacatctcttcatcggtaagtttagacagaatatcgtgaacaggtggtatattatattcatcagatatacgaagaacaatgtccaaatctatattgtttaatatattatatagatgtagcgtagctcctacaggaatatctttaactaagtcaatgatttcatcaaccgttagatctattttaaagttaatcatataggcattgatttttaaaaggtatgtagccttgactacattctcattaattaaccattccaagtcactgtgtgtaagaagattatattctatcataagcttgactacatttggtcccgataccattaaagaattcttatgatataaggaaacagattttaggtactcatctactctacaagaattttggagagccttaacgatatcagtgacgtttattatttcaggaggaaagaatctaacattgagaatatcggaattaatagcttccagatacagtgattttggcaatagtccgtgtaatccataatccagtaacacgagctggtgcttgctagacaccttttcaatgtttaatttttttgaaataagctttgataaagccttcctcgcaaattccggatacatgaacatgtcggcgacatgattaagtattgttttttcattattttctcaacaagttctcaataccccaatagatgatagaatatcacccaatgcgtccatgttgtctatttccaacaggtcgctatatccaccaatagaagtttttccaaaaaagattctaggaacagttctaccaccagtaatttgttcaaaatagtcacgcaattcattttcgggtttaaattctttaatatcgacaatttcatacgctcctcttttgaaactaaacttatttagaatatccagtgcatttctacaaaaaggacatgtatacttgacaaaaattgtcactttgttattggccaacctttgttgtacaaattcctcggccattttaatatttaagtgatataaaactatctcgacttatttaactctttagtcgagatatatggacgcagatagctatatgatagccaactacagaaggcaaacgctataaaaaacataattacgacgagcatatttataaatatttttattcagcattacttgatatagtaatattaggcacagtcaaacattcaaccactctcgatacattaactctctcattttctttaacaaattctacaatatcttcgtaaaaagattcttgaaactttttagaatatctatcgactctagatgaaatagcgttcgtcaacatactatgttttgtatacataaaggcgcccattttaacagtttctagtgacaaaatgctagcgatcctaggatcctttagaatcacatagattgacgattcgtctctcttagtaactctagtaaaataatcatacaatctagtacgcgaaataatattatccttgacttgaggagatctaaacaatctagttttgagaacatcgataagttcatcgggaatgacatacatactatctttaatagaactcttttcatccagttgaatggattcgtccttaaccaactgattaatgagatcttctattttatcattttccagatgatatgtatgtccattaaagttaaattgtgtagcgcttctttttagtctagcagccaatactttaacatcactaatatcgatatacaaaggagatgatttatctatggtattaagaattcgtttttcgacatctgtcaaaaccaattcctttttgcctgtatcatccagttttccatcctttgtaaagaaattattttctactagactattaataagactgataaggattcctccataattgcacaatccaaactttttaacaaaactagactttacaagatctacaggaatgcgtaattcaggtttcttagcttgtgattttttcttttgtggacattttcttgtgaccaactcatctaccatttcattgattttagcagtgaaataagctttcaatgcacgggcactgatactattgaaaacgagttgatcttcaaattccgccatttaagttcaccaaacaacttttaaatacaaatatatcaatagtagtagaataagaactataaaaaaaataataattaaccaataccaaccccaacaaccggtattattagttgatgtgactgttttctcatcacttagaacagatttaacaatttctataaagtctgtcaaatcatcttccggagaccccataaatacaccaaatatagcggcgtacaacttatccatttatacattgaatattggcttttctttatcgctatcttcatcatattcatcatcaatatcaacaagtcccagattacgagccagatcttcttctacattttcagtcattgatacacgttcactatctccagagagtccgataacgttagccaccacttctctatcaatgattagtttcttgagtgcgaatgtaatttttgtttccgttccggatctatagaagacgataggtgtgataattgccttggccaattgtctttctcttttactgagtgattctagttcaccttctatagatctgagaatggatgattctccagtcgaaacatattctaccatggatccgtttaatttgttgatgaagatggattcatccttaaatgttttctctgtaatagtttccaccgaaagactatgcaaagaatttggaatgcgttccttgtgcttaatgtttccatagacggcttctagaagttgatacaacataggactagccgcggtaacttttatttttagaaagtatccatcgcttctatcttgtttagatttatttttataaagtttagtctctccttccaacataataaaagtggaagtcatttgactagataaactatcagtaagttttatagagatagacgaacaattagcgtattgagaagcatttagtgtaacgtattcgatacattttgcattagatttactaatcgattttgcatactctataacacccgcacaagtctgtagagaatcgctagatgcagtaggtcttggtgaagtttcaactctcttcttgattaccttactcatgattaaacctaaataattgtactttgtaatataatgatatatattttcactttatctcatttgagaataaaaatgtttttgtttaaccactgcatgatgtacagatttcggaatcgcaaaccaccagtggttttattttatccttgtccaatgtgaattgaatgggagcggatgcgggtttcgtacgtagatagtacattcccgtttttagaccgagactccatccgtaaaaatgcatactcgttagtttggaataactcggatctgctatatggatattcatagattgactttgatcgatgaaggctcccctgtctgcagccatttttatgatcgtcttttgtggaatttcccaaatagttttataaactcgcttaatatcttctggaaggtttgtattctgaatggatccaccatctgccataatcctattcttgatctcatcattccataattttctctcggttaaaactctaaggagatgcggattaactacttgaaattctccagacaatactctccgagtgtaaatattactggtatacggttccaccgactcattatttcccaaaatttgagcagttgatgcagtcggcataggtgccaccaataaactatttctaagaccgtatgttctgattttatcttttagaggttcccaattccaaagatccgacggtacaacattccaaagatcatattgtagaataccgttactggcgtacgatcctacatatgtatcgtatggtccttccttctcagctagttcacaactcgcctctaatgcaccgtaataaatggtttcgaagatcttcttatttagatcttgtgcttccaggctatcaaatggataatttaagagaataaacgcgtccgctaatccttgaacaccaataccgataggtctatgtctcttattagagatttcagcttctggaataggataataattaatatctataattttattgagatttctgacaattactttgaccacatccttcagtttgagaaaatcaaatcgcccatctattacaaacatgttcaaggcaacagatgccagattacaaacggctacctcattagcatccgcatattgtattatctcagtgcaaagattactacacttgatagttcctaaattttgttgattactctttttgttacacgcatccttataaagaatgaatggagtaccagtttcaatctgagattctataatcgctttccagacgactcgagcctttattatagatttgtatctcctttctctttcgtatagtgtatacaatcgttcgaactcgtctccccaaacattgtccaatccaggacattcatccggacacatcaacgaccactctccgtcatccttcactcgtttcataaagagatcaggaatccaaagagctataaatagatctctggttctatgttcctcgtttcctgtattctttttaagatcgaggaacgccataatatcagaatgccacggttccaagtatatggccataactccaggccgtttgtttcctccctgatctatgtatctagcggtgttattataaactctcaacattggaataataccgtttgatataccattggtaccggagatatagcttccactggcacgaatattactaattgatagacctattccccctgccattttagagattaatgcgcatcgttttaacgtgtcatagataccctctatgctatcatcgatcatgttaagtagaaaacagctagacatttggtgacgactagttcccgcattaaataaggtaggagaagcgtgcgtaaaccatttttcagaaagtagattgtacgtctcaatagctgagtctatatcccattgatgaattcctactgcgacacgcattaacatgtgctgaggtctttcaacgatcttgttgtttattttcaacaagtaggatttttccaaagttttaaaaccaaaatagttgtatgaaaagtctcgttcgtaaataataaccgagttgagtttatccttatatttgttaactatatccatggtgatacttgaaataatcggagaatgtttcccatttttaggattaacatagttgaataaatcctccatcacttcactaaatagtttttttgtttccttgtgtagatttgatacggctattctggcggctagaatggcataatccggatgttgtgtagtacaagtggctgctatttcggctgccagagtgtccaattctaccgttgttactccattatatattccttgaataaccttcatagctattttaataggatctatatgatccgtgtttaagccataacataattttctaatacgagacgtgattttatcaaacatgacattttccttgtatccatttcgtttaatgacaaacatttttgttggtgtaataaaaaaaattatttaacttttcattaatagggatttgacgtatgtagcgtacaaaattatcgttcctggtatatagataaagagtcctatatatttgaaaatcgttacggctcgattaaactttaatgattgcatagtgaatatatcattaggatttaactccttgactatcatggcggcgccagaaattaccatcaaaagcattaatacagttatgccgatcgcagttagaacggttatagcatccaccatttatatctaaaaattagatcaaagaatatgtgacaaagtcctagttgtatactgagaattgacgaaacaatgtttcttacatattttttttttattagtaaccgacttaatagtaggaactggaaaactagacttgattattctataagtatagatacccttccaaataatattctctttgataaaagttccagaaaatgtagaattttttaaaaagttatcttttgctattaccaagattgtgtttagacgcttattattaatatgagtgatgaaatccacaccgcctctagatatcgcttttatttccacattagatggtaaatccaatagtgaaactatctttttaggaatgtatggactcgcgtttagaggagtgaacgtcttaggcgtcggaaaggatgattcatcaaacgaataaacaatttcacaaatggatgttaatgtattagtaggaaattttttgacgctagtggaattgaagattctaatggatgatgttctacctatttcatccgataacatgttaatttccgacaccaacggttttaatatttcgatgatatacggtagtctctctttcggacttatatagcttattccacaatacgagtcattatatactccaaaaaacaaaataactagtataaaatctgtatcgaatgggaaaaacgaaattatcgacataggtatagaatctggaacattgaacgtattaatacttaattctttttctgtggtaagtaccgataggttattgacattgtatggttttaaatattctataacttgagacttgatagatattagtgatgaattgaaaattatttttatcaccacgtgtgtttcaggatcatcgtcgacgcccgtcaaccaaccgaatggagtaaaataaatatcattaatatatgctctagatattagtatttttatcaatcctttgattatcatcttctcgtaggcgaatgattccatgatcaagagtgatttaagaacatcctccggagtattaatgggcttagtaaacagtccatcgttgcaataataaaagttatccaagttaaaggatattatgcattcgtttaaagatatcacctcatctgacggagacaattttttggtaggttttagagactttgaagctacttgtttaacaaagttattcatcgtcgtttactattctatttaattttgtagttaatttatcacatatcacattaattgactttttggtccatttttccatacgtttatattcttttaatcctgcgttatccgtttccgttatatccagggatagatcttgcaagttaaatagaatgctcttaaataatgtcattttcttatccgctaaaaatttaaagaatgtataaacctttttcagagatttgaaactcttaggtggtgtcctagtacacaatatcataaacaaactaataaacattccacattcagattccaacagctgattaacttctacattaatacagcctattttcgctccaaatgtacattcgaaaaatctgaataaaacatcgatgtcacaatttgtattatccaatacagaatgtttgtgattcgtgttaaaaccatcggagaaggaataaaaataaaaattattatagtggtggaattcagttggaatattgcctccggagtcataaaaggatactaaacattgttttttatcataaattacacatttccaatgagacaaataacaaaatccaaacattacaaatctagaggtagaacttttaattttgtctttaagtatatacgataagatatgtttattcataaacgcgtcaaatttttcatgaatcgctaaggagtttaagaatctcatgtcaaattgtcctatataatccacttcggatccataagcaaactgagagactaagttcttaatacttcgattgctcatccaggctcctctctcaggctctattttcatcttgacgacctttggattttcaccagtatgtattcctttacgtgataaatcatcgattttcaaatccatttgtgagaagtctatcgccttagatactttttcccgtagtcgaggtttaaagaaatacgctaacggtatactagtaggtaactcaaagacatcatatatagaatggtaacgcgtctttaactcgtcggttaactctttcttttgatcgagttcgtcgctactattgggtctgctcaggtgccccgactctactagttccaacatcataccgataggaatacaagacactttgccggcggttgtagatttatcatatttctccactacatatccgttacaatttgttaaaaatttagatacatctatattgctacataatccagctagtgaatatatatgacataataaattggtaaatcctagttctggtattttactaattactaaatctgtatatctttccatttatcatggaaaagaatttaccagatatcttcttttttccaaactgcgttaatgtattctcttacaaatattcacaagatgaattcagtaatatgagtaaaacggaacgtgatagtttctcattggcggtgtttccagttataaaacatagatggcataacgcacacgttgtaaaacataaaggaatatacaaagttagtacagaagcacgtggaaaaaaagtatctcctccatcactaggaaaacccgcacacataaacctaaccgcgaagcaatatatatacagtgaacacacaataagctttgaatgttatagttttctaaaatgtataacaaatacagaaatcaattcgttcgatgagtatatattaagaggactattagaagctggtaatagtttacagatattttccaattccgtaggtaaacgaacagatactataggtgtactagggaataagtatccatttagcaaaattccattggcctcattaactcctaaagcacaacgagagatattttcagcgtggatttctcatagacctgtagttttaactggaggaactggagtgggtaagacgtcacaggtacccaagttattgctttggtttaattatttatttggtggattctctactctagataaaatcactgactttcacgaaagaccagtcattctatctcttcctaggatagctttagttagattgcatagcaataccattttaaaatcattgggatttaaggtactagatggatctcctatttctttacggtacggatctataccggaagaattaataaacaaacaaccaaaaaaatatggaattgtattttctacccataagttatctctaacaaaactatttagttatggcactcttattatagacgaagttcatgagcatgatcaaataggagatattattatagcagtagcgagaaagcatcatacgaaaatagattctatgtttttaatgactgccacgttagaggatgacagggaacggctaaaagtatttttacctaatcccgcatttatacatattcctggagatacactgtttaaaattagcgaggtatttattcataataagataaatccatcttccagaatggcatacatagaagaagaaaagagaaatttagttactgctatacagatgtatactcctcctgatggatcatccggtatagtctttgtggcatccgttgcacagtgtcacgaatataaatcatatttagaaaaaagattaccgtatgatatgtatattattcatggtaaggtcttagatatagacgaaatattagaaaaagtgtattcatcacctaatgtatcgataattatttctactccttatttggaatccagcgttactatacgcaatgttacacacatttatgatatgggtagagtttttgtccccgctccttttggaggatcgcaagaatttatttctaaatctatgagagatcaacgaaaaggaagagtaggaagagttaatcctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaatttttatctagtataaaaaggcgcgccttatgtgtaatgtaatttgactcctttgagcactggctcagagtcgtcttcatcaaatttgcagcaggatccacaagaacaacagcccttgagacaactacagcaactggtcatacagcaaagcataattgtcaccattactatggcaatcaagccagctatgaaacctagccaaatgtaccatggccatttgatatactgctcatactttccaagttcttggagatcgatgagagattcatttaaattcttggcaacctcattgaggcggtcaatttctttctgaatgtttacaactgaagcattaatgccagagatgtcacctaaatcaacatctggtgatgtatgattcttgaaatatttatctaactcctccttgaatgagtctaattcaggttgcaaaggatcataaactgtgttgttgacaattcctattacaacatcacagttaccagacacaaatgtgttgtctgtagtaatgatttgtggttcatagaaattcctttgtgttacaaaccagtgtgtgccatttgaaacaaagacaccttcacgaggaaagtgtgctttaccatcatgacaaatggcaggagcagttgtgaagttcttctcttgtgcagggacataagtcacatgcaagaagactacaccatgaggtgctgactgagggaaggacataagatgatagccctttccacagaaatcaactctcttagattgtccaagtacacactctgacatcttagtagcagcaagattagcagaagctctgatttctgcagctctaattaattgttgagtcacatatgtctgcaaactttgaagtctgcctgtgatcaacctatcaatttgcacttcagcctcaactttgtcaagacgtgaaaggatatcatttaatacacttgaaattgcaccaaagttggagctaagttgtttaacaagcgtgtttaaagcttgtgcattctggttgaccacatcttgaagtttaccaagtgcacttgctgtggaagaaagtgagtcttgaatcttgccaatagcactattaaattggttggcaatcaatttctggttctcatagagaacattctgtgtaactccaataccattaaacctataagccatttgcatagcaaatggtatttgtaatgcagcacctgcaccaaaggtccaaccagaagtgattgtacccgctaacagtgcagaagtgtattgagcaatcatttcatctgtgagcaaaggtggcaatacagtaaggccgttaaacttctgtgcacaaatgaggtctctagcagcaatatcaccaaggcaatcaccatattgtttgatgaagccagcatctgcaagtgtcactttgttgaatagtagatcttcaataaatgacctcttgcttggtttagatggatctggtaatatttgtgagaaattaaatccaccgaaatctttaattggtggtgtcttgtaaatttgtttgacttgtgcaaatacttcttgggtgttcttgtcttgttcaacagctattccagttaaagcacggtttaattgtgtacagaaactgccatattgcaacaatagattgctgcattcagttgaatcaccacaaatgtacattgtacaatctactgatgtcttggtcatagacactggtagaatctctgtggtaacactaatagtaaagtttgtgggtatggcaatagagttattagagtaagcaactgaattctctgcaccaagtgacatagtgtaggcaatgatggattgactagctacactacgtgcccgccgaggagaattagtctgagtctgataactagcgcatatacctgcaccaatgggtatgtcacactcatatgagttgttgacatgttcagctcctattaaacagcctgcacgtgtttgaaatacattagaacctgtagaataaacacgccaagtaggagtaagttgatctgcatgaatagcaacagggacttctgtgcagttaacatcctgataaagaacagcaacctggttagaagtatttgttcctggtgttataacactgacaccaccaaatgaacatggtgtaatgtcaagaatctcaagtgtctgtggatcacggacagcatcagtagtgtcagcaatgtctctgccaaattgttggaaaggcagaaacttcttgttagactcagtaagaacacctgtgcctgttaaaccattgaagttgaaattgacacatttgttcttaaccaaattagtagacttcttaggtccacaaacagttgctggtgcatgtagaagttcaaatgaaagtactactactctgtatggttggtaaccaacaccattagtgggttggaaaccatatgattgtaaaggaaagtaacaattaaatccttcaacaccattacaaggtgtgctaccggcctgatagatttcagttgaaatatctctctcgaaaggtttgagattagacttcctaaacaatctatacaggtaattataattaccaccaaccttagaatcaagattgttagaattccaagctataacgcagcctgtaaagtcatctggtaatttataattataatcagcaatctttccagtttgccctggagcgatttgtctgacttcatcacctctaattacaaatgaatctgcatagacattagtaaagcagagatcatttaatttagtaggagacactccataacacttaaatgtggagaatgatgcggaattatataggacagaataatcagcaacacagttgctgattctcttcctgttccaagcataaacagatgcaaatctggtggcgttaaatacttcaccgaaagggcacaagtttgtaatattaggaaatctaacaatagattctgttggttggactctaaagttagaagtttgatagattcctttctctacagtgaaggatttcaacgtacactttgtttctgagagagggtcaagtgcacagtctacagcatctgtaatggttccattctcattatatttcaataggaaagtcctaggttgaagataacccacataataagctgcagcaccagctgtccaacctgaagaagaatcaccaggagtcaaataacttctatgtaaagcaagtaaagtttgaaacctagtgatgttaatacctattggcaaatctaccaatggttctaaagccgagaaaccctgagggagatcacgcactaaattaataggcgtgtgcttagaatatatcttgaaataaccatcaatattcttaaacacaaattccctaagattcttgaaattaccctgtttaccttcaaggtccataaggaaaggctgagagacatattcgaaagtgcaattattcgcactagaataaactctgaactcactttccatccaactcttgttgttcttgtggtaataaacacccaagaatggatcattacagaattgaaattcacagactttaataacaacattagtagcgttattaacaataagtagggactgggtcttcgaatctaaagtagtaccaaatatccagcctcttattatgttagacttctcagtggaagcgaaataaacaccatcattaaatggtaggacagggttatcaaacctcttagtaccattggtcccagagacatgtatagcatggaaccaagtaacattggagaagaaaggtaagaacaagtcctgagttgaatgtaatactgaggatctgaatactttgtcagggtaataaacaccacgtgtgaaagaattagtgtatgcaggtggtaattgagttctggttgtaagattaacacactgactagagactagtggcaataagacaagaaagacaaacatggtggcggcgtttatcttatttatgattatttctcgctttcaatttaacacaaccctcaagaacctttgtatttattttcaatttttataaaaatcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttgggtatctctagtgttacacatggccttactaaagtttgggtaaataactatgatatctctattaattatagatgcatatatttcatttgtcaaggatattagtatcgacttgctatcgtcattaatacgtgtaatgtaatcatataaatcatgcgatagccaaggaaaatttaaatagatgttcatcatataatcgtcgctataattcatattaatacgttgacattgactaatttgtaatatagcctcgccacgaagaaagctctcgtattcagtttcatcgataaaggataccgttaaatataactggttgccgatagtctcatagtctattaagtggtaagtttcgtacaaatacagaatccctaaaatattatctaatgttggattaatctttaccataactgtataaaatggagacggagtcataactattttaccgtttgtacttactggaatagacgaaggaataatctccggacatgctggtaaagacccaaatgtctgtttgaagaaatccaatgttccaggtcctaatctcttaacaaaaattacgatattcgatcccgatatcctttgcattctatttaccagcatatcacgaactatattaagattatctatcatgtctattctcccaccgttatataaatcgcctccgctaagaaacgttagtatatccatacaatggaatacttcatttctaaaatagtattcgttttctaattctttaatgtgaaatcgtatactagaaagggaaaaattatctttgagttttccgttagaaaagaaccacgaaactaatgttctgattgcgtccgattccgttgctgaattaatggatttacaccaaaaactcatataacttctagatgtagaagcattcgctaaaaaattagtagaatcaaaggatataagtagatgttccaacaagtgagcaattcccaagatttcatctatatcattctcgaatccgaaattagaaattcccaagtagatatcctttttcatccgatcgttgatgaaaatacgaactttattcggtaagacaatcatttactaaggagtaaaataggaagtaatgttcgtatgtcgttatcatcgtataaattaaaggtgtgttttttaccattaagtgacattataattttaccaatattggaattataatataggtgtatttgcgcactcgcgacggttgatgcatcggtaaatatagctgtatctaatgttctagtcggtatttcatcatttcgctgtctaataatagcgttttctctatctgtttccattacagctgcctgaagtttattggtcggataatatgtaaaataataagaaatacatacgaataacaaaaataaaataagatataataaagatgccatttagagatctaattttgtttaacttgtccaaattcctacttacagaagatgaggaatcgttggagatagtgtcttccttatgtagaggatttgaaatatcttatgatgacttgataacttactttccagataggaaataccataaatatatttctaaagtatttgaacatgtagatttatcggaggaattaagtatggaattccatgatacaactctgagagatttagtctatcttagattgtacaagtattccaagtgtatacggccgtgttataaattaggagataatctaaaaggcatagttgttataaaggacaggaatatttatattagagaagcaaatgatgacttgatagaatatctcctcaaggaatacactcctcagatttatacatattctaatgagcgcgtccccataactggttcaaaattaattctttgtggattttctcaagttacatttatggcgtatacaacgtcgcatataacaacaaataaaaaggtagatgttctcgtttccaaaaaatgtatagatgaactagtcgatccaataaattatcaaatacttcaaaatttatttgataaaggaagcggaacaataaacaaaatactcaggaagatattttattcggtaaccggtggccaaactccataatttgctttttctatttcggattttagaatttccaaattcaccagcgatttatcggttttggtgaaatccaaggatttattaatgtccacaaatgccatttgttttgtctgtggattgtatttgaaaatggaaacgatgtagttagatagatgcgctgcaaagtttcctattagggttccgcgctttacgtcacccagcatacttgaatcaccatcctttaaaaaaaatgataagatatcaacatggagtatatcatactcggattttaattcttctactgcatcactgacattttcacaaatactacaatacggtttaccgaaaataatcagtacgttcttcatttatgggtatcaaaaacttaaaatcgttactgctggaaaataaatcactgacgatattagatgataatttatacaaagtatacaatggaatatttgtggatacaatgagtatttatatagccgtcgccaattgtgtcagaaacttagaagagttaactacggtattcataaaatacgtaaacggatgggtaaaaaagggagggcatgtaaccctttttatcgatagaggaagtataaaaattaaacaagacgttagagacaagagacgtaaatattctaaattaaccaaggacagaaaaatgctagaattagaaaagtgtacatccgaaatacaaaatgttaccggatttatggaagaagaaataaaggcagaaatgcaattaaaaatcgataaactcacatttcaaatatatttatctgattctgataacataaaaatatcattgaatgagatactaacacatttcaacaataatgagaatgttacattattttattgtgatgaacgagacgcagaattcgttatgtgtctcgaggctaaaacacatttctctaccacaggagaatggccgttgataataagtaccgatcaggatactatgctatttgcatctactgataatcatcctaagatgataaaaaacttaactcaactgtttaaatttgttccctcggcagaggataactatttagcaaaattaacggcgttagtgaatggatgtgatttctttcctggactctatggggcatctataacacccaccaacttaaacaaaatacaattgtttagtgattttacaatcgataatatagtcactagtttggcaattaaaaattattatagaaagactaactctaccgtagacgtgcgtaatattgttacgtttataaacgattacgctaatttagacgatgtctactcgtatgttcctccttgtcaatgcactgttcaagaatttatattttccgcattagatgaaaaatggaacaattttaaatcatcttatttagagaccgttccgttaccctgccaattaatgtatgcattagaaccacgcaaggagattgatgtttcagaagttaaaactttatcatcttatatagatttcgaaaatactaaatcagatatcgatgttataaaatctatatcttcgatcttcggatattctaacgaaaactgtaacactatagtgttcggcatctataaggataatttactactgagtataaatagttcattttactttaacgatagtctgttaataaccaatactaaaagtgataatataataaatataggttactagattaaaaatggtgttccaactcgtgtgctctacgtgcggcaaagatatttctcacgaacgatataaattgattatacgaaaaaaatcattaaaggatgtactcgtcagtgtaaagaacgaatgttgtaggttaaaattatctacacaaatagaacctcaacgtaacttaacagtgcaacctctattggatataaactaatatggatccggttaattttatcaagacatatgcgcctagaggttctattatttttattaattataccatgtcattaacaagtcatttgaatccatcgatagaaaaacatgtgggtatttattatggtacgttattatcggaacacttggtagttgaatctacctatagaaaaggagttcgaatagtcccattggatagtttttttgaaggatatcttagtgcaaaagtatacatgttagagaatattcaagttatgaaaatagcagctgatacgtcattaactttattgggtattccgtatggatttggtcataatagaatgtattgttttaaattggtagctgaatgttataaaaatgccggtattgatacatcgtctaaacgaatattaggtaaagatatttttctgagccaaaacttcacagatgataatagatggataaagatatatgattctaataatttaacattttggcaaattgattaccttaaagggtgagttaatatgcataactactcctccgttgttttttccctcgttctttttcttaacgttgtttgccatcactctcataatgtaaagatattctaaaatggtaaacttttgcatatcggacgcagaaattggtataaatgttgtaattgtattatttcccgtcaatggactagtcacagctccatcagttttatatcctttagagtatttctcactcgtgtctaacattctagagcattccatgatctgtttatcgttgatattggccggaaagatagattttttattttttattatattactattggcaattgtagatataacttctggtaaatatttttctaccttttcaatctcttctattttcaagccggctatatattctgctatattgttgctagtatcaataccttttctggctaagaagtcatatgtggtattcactatatcagttttaactggtagttccattagcctttccacttctgcagaataatcagaaattggttctttaccagaaaatccagctactataataggctcaccgatgatcattggcaaaatcctatattgtaccagattaatgagagcatatttcatttccaataattctgctagttcttgagacattgatttatttgatgaatctagttggttctctagatactctaccatttctgccgcatacaataacttgttagataaaatcagggttatcaaagtgtttagcgtggctagaatagtgggcttgcatgtattaaagaatgcggtagtatgagtaaaccgttttaacgaattatatagtctccagaaatctgtggcgttacatacatgagccgaatgacatcgaagattgtccaatatttttaatagctgctctttgtccattatttctatatttgactcgcaacaattgtagataccattaatcactgattcctttttcgatgccggacaatagcacaattgtttagctttggactctatgtattcagaattaatagatatatctctcaatacagattgcactatacattttgaaactatgtcaaaaattgtagaacgacgctgttctgcagccatttaactttaaataatttacaaaaatttaaaatgagcatccgtataaaaatcgataaactgcgccaaattgtggcatatttttcagagttcagtgaagaagtatctataaatgtagactcgacggatgagttaatgtatatttttgccgccttgggcggatctgtaaacatttgggccattatacctctcagtgcatcagtgttctaccgcggagccgaaaacattgtgtttaatcttcctgtgtccaaggtaaaatcgtgtttgtgtagttttcacaatgatgccatcatagatatagaacctgatctggaaaataatctagtaaaactttctagttatcatgtagtaagtgtcgattgtaacaaggaactgatgcctattaggacagatactactatttgtctaagtatagatcaaaagaaatcttacgtgtttaattttcacaagtatgaagaaaaatgttgtggtagaaccgtcattcatttagaatggttgttgggctttatcaagtgtattagtcagcatcagcatttggctattatgtttaaagatgacaatattattatgaagactcctggtaatactgatgcgttttccagggaatattctatgactgaatgttctcaagaactacaaaagttttctttcaaaatagctatctcgtctctcaacaaactacgaggattcaaaaagagagtcaatgtttttgaaactagaatcgtaatggataatgacgataacattctaggaatgttgttttcggatagagttcaatcctttaagatcaacatctttatgacgtttttagattaatactttcaatgagataaatatgggtggcagagtaagtgttgagctccctaaacgggatccgcctccgggagtacccactgatgagatgttattaaacgtggataaaatgcatgacgtgatagctcccgctaagcttttagaatatgtgcatataggaccactagcaaaagataaagaggataaagtaaagaaaagatatccagagtttagattagtcaacacaggacccggtggtctttcggcattgttaagacaatcgtataatggaaccgcacccaattgctgtcgcacttttaatcgtactcattattggaaaaaggatggaaagatatcagataagtatgaagagggtgcagtattagaatcgtgttggccagacgttcacgacactggaaaatgcgatgttgatttattcgactggtgtcagggggatacgttcgatagaaacatatgccatcagtggatcggttcagcctttaataggagtgatagaactgtagagggtcaacaatcgttaataaatctgtataataagatgcaaacattatgtagtaaagatgctagtgtaccaatatgtgaatcatttttgcatcatttacgcgcacacaatacagaagatagcaaagagatgatcgattatattctaagacaacagtctgcggactttaaacagaaatatatgagatgtagttatcccactagagataagttagaagagtcattaaaatatgcggaacctcgagaatgttgggatccagagtgttcgaatgccaatgttaatttcttactaacacgtaattataataatttaggactttgcaatattgtacgatgtaatactagcgtgaacaacttacagatggataaaacttcctcattaagattgtcatgtggattaagcaatagtgatagattttctactgttcccgtcaatagagcaaaagtagttcaacataatattaaacattcgttcgacctaaaattgcatttgatcagtttattatctctcttggtaatatggatactaattgtagctatttaaatgggtgccgcggcaagcatacagacgacggtgaatacactcagcgaacgtatctcgtctaaattagaacaagaagcgaacgctagtgctcaaacaaaatgtgatatagaaatcggaaatttttatatccgacaaaaccatggatgtaacctcactgttaaaaatatgtgctctgcggacgcggatgctcagttggatgctgtgttatcagccgctacagaaacatatagtggattaacaccggaacaaaaagcatacgtaccagctatgtttactgctgcgttaaacattcagacgagtgtaaacactgttgttagagattttgaaaattatgtgaaacagacttgtaattctagcgcggtcgtcgataacaaattaaagatacaaaacgtaatcatagatgaatgttacggagccccaggatctccaacaaatttggaatttattaatacaggatctagcaaaggaaattgtgccattaaagcgttgatgcaattgacgactaaggccactactcaaatagcacctagacaagttgctggtacaggagttcagttttatatgattgttatcggtgttataatattggcagcgttgtttatgtactatgccaagcgtatgttgttcacatccaccaatgataaaatcaaacttattttagccaataaggaaaacgtccattggactacttacatggacacattctttagaacttctccgatggttattgctaccacggatatgcaaaactgaaaatatattgataatattttaatagattaacatggaagttatcgctgatcgtctagacgatatagtgaaacaaaatatagcggatgaaaaatttgtagattttgttatacacggtctagagcatcaatgtcctgctatacttcgaccattaattaggttgtttattgatatactattatttgttatagtaatttatatttttacggtacgtctagtaagtagaaattatcaaatgttgttggcgttggtggcgctagtcatcacattaactattttttattactttatactataatagtactagactgacttctaacaaacatctcacctgccataaataaatgcttgatattaaagtcttctatttctaacactattccatctgtggaaaataatactctgacattatcgctaattgacacatcggtgagtgatatgcctataaagtaataatcttctttgggcacatataccagtgtaccaggttctaacaacctatttactggtgctcctgtagcatactttttctttaccttgagaatatccatcgtttgcttggtcaatagcgatatgtgattttttatcaaccactcaaaaaagtaattggagtgttcatatcctctacgggctattgtctcatggccgtgtatgaaatttaagtaacacgactgtggtagatttgttctatagagccggttgccgcaaatagatagaactaccaatatgtctgtacaaatgttaaacattaattgattaacagaaaaaacaatgttcgttctgggaatagaaaccagatcaaaacaaaattcgttagaatatatgccacgtttatacatggaatataaaataactacagtttgaaaaataacagtatcatttaaacatttaacttgcggggttaatttcacaactttactgtttttaaactgttcaaaatatagcatcgatccatgagaaatacgtttagccgcctttaatagaggaaatcccaccgcctttctggatctcaccaacgacgatagttctgaccagcaacttatttcttcatcatccacctgttttaacatataataggcaggagatagatatccgtcattgcaatattccttttcgtaggcacacaatctaatattgataaaatctccattctcttctctgcatttattatcttgtttcggtggctgattaggctgtagtcttggtttaggctttggtatatcgttgttgaatctattttggtcattaaatctttcatttcttcctggtatatttctatcacctcgtttggttggatttttgtctatattatcgtttgtaacatcggtacgggtattcatttatcacaaaaaaaacttctctaaatgagtctactgctagaaaacctcatcgaagaagataccatattttttgcaggaagtatatctgagtatgatgatttacaaatggttattgccggcgcaaaatccaaatttccaagatctatgctttctatttttaatatagtacctagaacgatgtcaaaatatgagttggagttgattcataacgagaatatcacaggggcaatgtttaccacaatgtataatataagaaacaatttgggtctaggagatgataaactaactattgaagccattgaaaactatttcttggatcctaacaatgaggttatgcctcttatcattaataatacggatatgactgccgtcattcctaaaaaaagtggtaggagaaagaataagaacatggttattttccgtcaaggatcatcacctatcttgtgtattttcgaaactcgtaaaaagattaatatttataaagaaaatatggaatccgcgtcgactgagtatacacctatcggagacaacaaggctttgatatctaaatatgcgggaattaatgtcctgaatgtgtattctccttccacatccatgagattgaatgccatttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcattgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctattagatacatagatcctcgtcgcgatatcgcattttctaacgtgatggatatattaaagtcgaataaagtgaacaataattaattctttattgtcatcatgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagagataataggaggtaatgatatgtatcaatcggtgtgtagaaagtgttacgtcggctcataatattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacgtaaaatatctagcaaaaattctatgtctaaaaacagaaatagcaagagatccgtacgcagtaataaatagaaacgtactactgagatatactaccgatatagagtataatgatttagttactttaataaccgttagacataaaattgattctatgaaaactgtgtttcaggtatttaacgaatcatccataaattatactccggttgatgatgattatggagaaccaatcattataacatcgtatcttcaaaaaggtcataacaagtttcctgtaaattttctatacatagatgtggtaatatctgacttatttcctagctttgttagactagatactacagaaactaatatagttaatagtgtactacaaacaggcgatggtaaaaagactcttcgtcttcccaaaatgttagagacggaaatagttgtcaagattctctaccgtcctaatataccattaaaaattgttagatttttccgcaataacatggtaactggagtagagatagccgatagatctgttatttcagtcgctgattaatcaattagtagagatgagataagaacattataataatcaataatatattttatatcttatatcttgtttagaaaaatgctaatattaaaatagctaacgctagtaatccaatcggaagccatttgatatctataatagggtatctaatttcctgattcagatagcggacagctatattctcggtagctactcgtttggaatcacaaacattatttacatctaatttactatctgtaatggaaacgtttcccaatgaaatggtacaatccgatacattgcattttgttatatttttttttaaagaggctggtaacaacgcatcgcttcgtttacatggctcgtaccaacaataatagggtaatcttgtatctattcctatccgtactatgcttttatcaggataaatacatttacatcgtatatcgtctttgttagcatcacagaatgcataaatttgttcgtccgtcatgataaaaatttaaagtgtaaatataactattattttatagttgtaataaaaagggaaatttgattgtatactttcggttctttaaaagaaactgacttgataaaaatggctgtaatctctaaggttacgtatagtctatatgatcaaaaagagattaatgctacagatattatcattagtcatgttaaaaatgacgacgatatcggtaccgttaaagatggtagactaggtgctatggatggggcattatgtaaaacttgtgggaaaacggaattggaatgtttcggtcactggggtaaagtaagtatttataaaactcatatagttaagcctgaatttatttcagaaattattcgtttactgaattatatatgtattcactgcggattattgcgttcacgagaaccgtattccgacgatattaacctaaaagagttatcgggacacgctcttaggagattaaaggataaaatattatccaagaaaaagtcatgttggaacagtgaatgtatgcaaccgtatcaaaaaattactttttcaaagaaaaaggtttgtttcgtcaacaagttggatgatattaacgttcctaattctctcatctatcaaaagttaatttctattcatgaaaagttttggccattattagaaattcatcaatatccagctaacttattttatacagactactttcccatccctccgttgattattagaccggctattagtttttggatagatagtatacccaaagaaaccaatgaattaacttacttattaggtatgatcgttaagaattgtaacttgaatgctgatgaacaggttatccagaaggcggtaatagaatacgatgatattaaaattatttctaataacacttccagtatcaatttatcatatatcacatccggcaaaaataatatgattagaagttatatcgtcgcccggcgaaaagatcagaccgctagatctgtaattggtcccagtacatctatcaccgttaatgaggtaggaatgcccgcatatattagaaatacacttacagaaaagatatttgttaatgcctttacagtggataaagttaaacaactattagcgtcaaaccaagttaaattttactttaataaacgattaaaccaattaacaagaatacgccaaggaaagtttatcaaaaataaaatacatttattgcctggtgattgggtagaagtagctgttcaagaatatacaagtattatttttggaagacagccgtctctacatagatacaacgtcatcgcttcatctatcagagctaccgaaggagatactatcaaaatatctcccggaattgtcaactctcaaaatgctgatttcgacggagatgaagaatggatgatattggagcaaaatcctaaagccgtaattgaacaaagtattcttatgtatccgacgacgttactcaaacacgatattcatggagcccccgtttatggatctattcaagatgaaatcgtagcagcgtattcattgtttaggatacaagatctttgtttagatgaagtattgaacatcttggggaaatatggaagagagttcgatcctaaaggtaaatgtaaattcagcggtaaagatatctatacttacttgataggtgaaaagattaattatccgggtctcttaaaggatggtgaaattattgcaaacgacgtagatagtaattttgttgtggctatgaggcatctgtcattggctggactcttatccgatcataagtcgaacgtggaaggtatcaactttattatcaagtcatcttatgtttttaagagatatctatctatttacggttttggggtgacattcaaagatctgagaccaaattcgacgttcactaataaattggaggccatcaacgtagaaaaaatagaacttatcaaagaagcatacgccaaatatctcaacgatgtaagagacgggaaaatagttccattatctaaagctttagaggcggactatgtggaatccatgttatccaacttgacaaatcttaatatccgagagatagaagaacatatgagacaaacgctgatagatgatccagataataacctcctgaaaatggccaaagcgggttataaagtaaatcccacagaactaatgtatattctaggtacttatggacaacagaggattgatggtgaaccagcagagactcgagtattgggtagagtcttaccttactatcttccagactctaaggatccagaaggaagaggttacattcttaattctttaacaaaaggattaacgggttctcaatattacttttcgatgctggttgcaagatctcaatctactgatatcgtctgtgaaacatcacgtaccggaacactggctagaaaaatcattaaaaagatggaggatatggtggtcgacggatacggacaagtagttataggtaatacgctcatcaagtacgccgccaattataccaaaattctaggctcagtatgtaaacctgtagatcttatctatccagatgagtccatgacttggtatttggaaattagtgctctgtggaataaaataaaacagggattcgtttactctcagaaacagaaacttgcaaaaaagacattggcgccgtttaatttcctagtattcgtcaaacccaccactgaggataatgctattaaggttaaggatctgtacgatatgattcataacgtcattgatgatgtgagagagaaatacttctttacggtatctaatatagattttatggagtatatattcttgacgcatcttaatccttctagaattagaattacaaaagaaacggctatcactatctttgaaaagttctatgaaaaactcaattatactctaggtggtggaactcctattggaattatttctgcacaggtattgtctgagaagtttacacaacaagccctgtccagttttcacactactgaaaaaagtggtgccgtcaaacaaaaacttggtttcaacgagtttaataacttgactaatttgagtaagaataagaccgaaattatcactctggtatccgatgatatctctaaacttcaatctgttaagattaatttcgaatttgtatgtttgggagaattaaatccaaacatcactcttcgaaaagaaacagataggtatgtagtagatataatagtcaatagattatacatcaagagagcagaaattaccgaattagtcgtcgaatatatgattgaacgattcatctcctttagcgtcattgtaaaggaatggggtatggaaacattcattgaggatgaggataatattagatttactgtctacctaaatttcgttgaaccggaagaattgaatcttagtaagtttatgatggttcttccgggtgccgccaacaagggcaagattagtaaattcaagattcctatctctgattatacgggatatgacgacttcaatcaaacaaaaaagctcaataagatgactgtagaactcatgaatctaaaagaattgggttctttcgatttggaaaacgtcaacgtgtatcctggagtatggaatacatacgatatcttcggtatcgaggccgctcgtgaatacttgtgcgaagccatgttaaacacctatggagaagggttcgattatctgtatcagccttgtgatcttctcgctagtttactatgtgctagttacgaaccagaatcagtgaataaattcaagttcggcgcagctagtactcttaagagagctacgttcggagacaataaagcattgttaaacgcggctcttcataaaaagtcagaacctattaacgataatagtagctgccacttttttagcaaggtccctaatataggaactggatattacaaatactttatcgacttgggtcttctcatgagaatggaaaggaaactatctgataagatatcttctcaaaagatcaaggaaatggaagaaacagaagacttttaattcttatcaataacatatttttctatgatctgtcttttaaacgatggattttccacaaatgcgcctctcaagtccctcatagaatgatacacgtataaaaaatatagcataggcaatgactccttatttttagacattagatatgccaaaatcatagccccgcttctatttactcccgcagcacaatgaaccaacacgggctcgtttcgttgatcacatttagataaaaaggcggttacgtcgtcaaaatatttactaatatcggtagttgtatcatctaccaacggtatatgaataatattaatattagagttaggtaatgtatatttatccatcgtcaaatttaaaacatatttgaacttaacttcagatgatggtgcatccatagcatttttataatttcccaaatacacattattggttactcttgtcattatagtgggagatttggctttgtgcatatctccagttgaacgtagtagtaagtatttatacaaacttttcttatccatttataacgtacaaatggataaaactactttatcggtaaacgcgtgtaatttagaatacgttagagaaaaggctatagtaggcgtacaagcagccaaaacatcaacacttatattctttgttattatattggcaattagtgcgctattactctggtttcagacgtctgataatccagtctttaatgaattaacgagatatatgcgaattaaaaatacggttaacgattggaaatcattaacggatagcaaaacaaaattagaaagtgatagaggtagacttctagccgctggtaaggatgatatattcgaattcaaatgtgtggatttcggcgcctattttatagctatgcgattggataagaaaacatatctgccgcaagctattaggcgaggtactggagacgcgtggatggttaaaaaggcggcaaaggtcgatccatctgctcaacaattttgtcagtatttgataaaacacaagtctaataatgttattacttgtggtaatgagatgttaaatgaattaggttatagcggttattttatgtcaccgcattggtgttccgattttagtaatatggaatagtgttagataaatgcggtaacgaatgttcctgtaaggaaccataacagcttagatttaacgttaaagatgagcataaacataataaacaaaattacaatcaaacctataacattaatatcaaacaatccaaaaaatgaaatcagtggagtagtaaacgcgtacataactcctggataacgtttagcagctgccgttcctattctagaccaaaaattcggtttcatgttttcgaaacggtattctgcaacaagtcgaggatcgtgttctacatatttggcggcgttatccagtatctgcctattgatcttcatttcgttttcgattctggctatttcaaaataaaatcccgatgatagacctccagactttataatttcatctacgatgttcagcgccgtagtaactctaataatataggctgataagctaacatcataccctcctgtatatgtgaatatggcatgatttttgtccattacaagctcggttttaactttattgcctgtaataatttctctcatctgtaggatatctatttttttgtcatgcattgccttcaagacgggacgaagaaacgtaatatcctcaataacgttatcgttttctacaataactacatattctacctttttattttctaactcggtaaaaaaattagaatcccatagggctaaatgtctagcgatatttcttttcgtttcctctgtacacatagtgttacaaaaccctgaaaagaagtgagtatacttgtcatcatttctaatgtttcctccagtccactgtataaacgcataatccttgtaatgatctggatcatccttgactaccacaacatttcttttttctggcataacttcattgtcctttacatcatcgaacttctgatcattaatatgctcatgaacattaggaaatgtttctgatggaggtctatcaataactggcacaacaataacaggagttttcaccgccgccatttagttattgaaattaatcatatacaactctttaatacgagttatattttcgtctatccattgtttcacatttacatatttcgacaaaaagatataaaatgcgtattccaatgcttctctgtttaatgaattactaaaatatacaaacacgtcactgtctggcaataaatgatatcttagaatattgtaacaatttattttgtattgcacatgttcgtgatctatgagttcttcttcgaatggcataggatctccgaatctgaaaacgtataaataggagttagaataataatatttgagagtattggtaatatataaactctttagcggtataattagtttttttctctcgatttctatttttagatgtgatggaaaaatgactaattttgtagcattagtatcatgaactctaatcgagatcttaatatcttcgtcacacgttagttctttgaagtttttaagagatgcatcagttggttcgaccgatggagtaggtgcaacaattttttgttcgatgtatgtatgtactggagccattgtcttaactataatggtgcttgtatcgaaaaactttaatgcagataatggaagctcttcgccgcgactttctacatcgtaattgggttctaacgccgatctctgaatggatactagttttctaagttctaatgtgattctctgaaaatgtaaatccaattcctccggcattatagatgtgtatacatcggtaaataaaactatagtatccaacgatcccttctcgcaaattctagtcttaaccaaaaaatcgtatataaccacggagatggcgtatttaagagtggattcttctaccgttttgttcttggatttcatataagaaactataaagtccgcactactgttaagaatgattactaacgcaactatatagtttaaattaagcatcttggaaacataaaataactctgtagacgatacttgactttcgaataagtttgcagacaaacgaagaaagaacagacctctcttaatttcagaagaaaactttttttcgtattcctgacgtctagagtttatatcaataagaaagttaagaattagtcggttaatgttgtatttcattacccaagtttgagatttcataatattatcaaaagacatgataatattaaagataaagcgctgactatgaacgaaatagctatatggttcgctcaagaatatagtcttgttaaacgtggaaacgataactgtatttttaatcacgtcagcggcatctaaattaaatataggtatatttattccacacactctacaatatgccacaccatcttcataataaataaattcgttagcaaaattattaattttagtgaaatagttagcgtcaactttcatagcttccttcaatctaatttgatgctcacacggtgcgaattccactctaacatcccttttccatgcctcaggttcatcgatctctataatatctagttttttgcgtttcacaaacacaggctcgtctctcgcgatgagatctgtatagtaactatgtaaatgataactagatagaaagatgtagctatatagatgacgatcctttaagagaggtatgatgactttaccccaatcagatagactgttgttatggtcttcggaaaaagaatttttataaatttttccagtattttccaaatatacgtacttaacatctaaaaaatccttaatgataataggaatggataatccgtctattttataaagaaatacatatcgcacattatacttttttttggaaatgggaataccgatgtgtctacataaatatgcaaagtctaaatattttttagagaatcttagttggtccaaattcttttccaagtacggtaatagatttttcatattgaacggtatcttcttaatctctggttctagttccgcattaaatgatgaaactaagtcactatttttataactaacgattacatcacctctaacatcatcatttaccagaatactgatcttcttttgtcgtaaatacatgtctaatgtgttaaaaaaaagatcatacaagttatacgtcatttcatctgtggtattcttgtcattgaaggataaactcgtactaatctcttctttaacagcctgttcaaatttatatcctatatacgaaaaaatagcaaccagtgtttgatcatccgcgtcaatattctgttctatcgtagtgtataacaatcgtatatcttcttctgtgatagtcgatacgttataaaggttgataacgaaaatatttttatttcgtgagataaagtcatcgtaggattttggacttatattcgcgtctagtagatatgcttttatttttggaatgatctcaattagaatagtctctttagagtccatttaaagttacaaacaactaggaaattggtttatgatgtataatttttttagtttttatagattctttattctatacttaaaaaatgaaaataaatacaaaggttcttgagggttgtgttaaattgaaagcgagaaataatcataaattatttcattatcgcgatatccgttaagtttgtatcgtaatggcgtggtcaattacaaataaagcggatactagtagcttcacaaagatggctgaaatcagagctcatctaaaaaatagcgctgaaaataaagataaaaacgaggatattttcccggaagatgtaataattccatctactaagcccaaaaccaaacgagccactactcctcgtaaaccagcggctactaaaagatcaaccaaaaaggaggaagtggaagaagaagtagttatagaggaatatcatcaaacaactgaaaaaaattctccatctcctggagtcagcgacattgtagaaagcgtggctgctgtagagctcgatgatagcgacggggatgatgaacctatggtacaagttgaagctggtaaagtaaatcatagtgctagaagcgatctttctgacctaaaggtggctaccgacaatatcgttaaagatcttaagaaaattattactagaatctctgcagtatcgacggttctagaggatgttcaagcagctggtatctctagacaatttacttctatgactaaagctattacaacactatctgatctagtcaccgagggaaaatctaaagttgttcgtaaaaaagttaaaacttgtaagaagtaaatgcgtgcacttttttataaagatggtaaactctttaccgataataattttttaaatcctgtatcagacgataatccagcgtatgaggttttgcaacatgttaaaattcctactcatttaacagatgtagtagtatatgaacaaacgtgggaggaggcgttaactagattaatttttgtgggaagtgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataaaccgctattgaaactgacggatgattctagtcccgaagaatttctgttcaacaaactaagtgaacgaaaggtatacgaatgtatcaaacagtttggtattagaatcaaggatctccgaacgtatggagtcaattatacgtttttatataatttttggacaaatgtaaagtccatatctcctcttccgtcaccaaaaaagttaatagcgttaactatcaaacaaactgctgaagtggtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatgatatagatctttacacaaataattacaagaccgataaatggaaatggataagcgtatgaaatctctcgcaatgacagctttcttcggagagctaaacacattagatattatggcattgataatgtctatatttaaacgccatccaaacaataccattttttcagtggataaggatggtcagtttatgattgatttcgaatacgataattataaggcttctcaatatttggatctgaccctcactccgatatctggagatgaatgcaagactcacgcatcgagtatagccgaacaattggcgtgtgtggatattattaaagaggatattagcgaatatatcaaaactactccccgtcttaaacgatttataaaaaaataccgcaatagatcagatactcgtatcagtcgagatacagaaaagcttaaaatagctctagctaaaggcatagattacgaatatataaaagacgcttgttaataagtaaatgaaaaaaaactagtcgtttataataaaacacaatatggatgccaacatagtatcatcttctactattgcaacgtatatagacgctttagcgaagaatgcttcagaattagaacagaggtctaccgcatacgaaataaataatgaattggaactagtatttattaagccgccattaattactttgacaaatgtagtgaatatctctacgattcaggaatcgtttattcgatttaccgttactaataaggaaggtgttaaaattagaactaagattccattatctaaggtacatggtctagatgtaaaaaatgtacagttagtagatgctatagataacatagtttgggaaaagaaatcattagtgacggaaaatcgtcttcacaaagaatgcttgttgagactatcgacagaggaacgtcatatatttttggattacaagaaatatggatcctctatccgactagaattagtcaatcttattcaagcaaaaacaaaaaactttacgatagactttaagctaaaatattttctaggatccggtgcccagtctaaaagttctttattacacgctattaatcatccaaagtcaaggcctaatacatctctggaaatagaattcacacctagagacaatgaaaaagttccatatgatgaactaataaaggaattgacgactctatcacgtcatatatttatggcttctccagagaatgtaattctttctccgcctattaacgcgcctataaaaacctttatgttgcctaaacaagatatagtaggtttggatctggaaaatctatatgccgtaactaagactgacggaattcctataactatcagagttacatcaaaagggttgtattgttattttacacatcttggttatattattagatatcctgttaagagaataatagattccgaagtagtagtctttggtgaggcagttaaggataagaactggaccgtatatctcattaagctaatagagcctgtgaatgcaatcaatgatagactagaagaaagtaagtatgttgaatctaaactagtggatatttgtgatcggatagtattcaagtcaaagaaatacgaaggtccgtttactacaactagtgaagtcgtcgatatgttatctacatatttaccaaagcaaccagaaggtgttattctgttctattcaaagggacctaaatctaacattgattttaaaattaaaaaggaaaatactatagaccaaactgcaaatgtagtatttaggtacatgtccagtgaaccaattatctttggagaatcgtctatctttgtagagtataagaaatttagcaacgataaaggctttcctaaagaatatggttctggtaagattgtgttatataacggcgttaattatctaaataatatctattgtttggaatatattaatacacataatgaagtgggtattaagtccgtggttgtacctattaagtttatagcagaattcttagttaatggagaaatacttaaacctagaattgataaaaccatgaaatatattaactcagaagattattatggaaatcaacataatatcatagtcgaacatttaagagatcaaagcatcaaaataggagatatctttaacgaggataaactatcggatgtgggacatcaatacgccaataatgataaatttagattaaatccagaagttagttattttacgaataaacgaactagaggaccgttgggaattttatcaaactacgtcaagactcttcttatttctatgtattgttccaaaacatttttagacgattccaacaaacgaaaggtattggcgattgattttggaaacggtgcggacctggaaaaatacttttatggagagattgcgttattggtagcgacggatccggatgctgatgctatagctagaggaaatgaaagatacaacaaattaaactctggaattaaaaccaagtactacaaatttgactacattcaggaaactattcgatccgatacatttgtctctagtgtcagagaagtattctattttggaaagtttaatatcatcgactggcagtttgctatccattattcttttcatccgagacattatgctaccgtcatgaataacttatccgaactaactgcttctggaggcaaggtattaatcactaccatggacggagacaaattatcaaaattaacagataaaaagacttttataattcataagaatttacctagtagcgaaaactatatgtctgtagaaaaaatagctgatgatagaatagtggtatataatccatcaacaatgtctactccaatgactgaatacattatcaaaaagaacgatatagtcagagtgtttaacgaatacggatttgttcttgtagataacgttgatttcgctacaattatagaacgaagtaaaaagtttattaatggcgcatctacaatggaagatagaccgtctacaaaaaactttttcgaactaaatagaggagccattaaatgtgaaggtttagatgtcgaagacttacttagttactatgttgtttatgtcttttctaagcggtaaataataatatggtatgggttctgatatccccgttctaaatgcattaaataattccaatagagcgatttttgttcctataggaccttccaactgtggatactctgtattgttaatagatatattaatacttttgtcgggtaacagaggttctacgtcttctaaaaataaaagtttgataacatctggcctgttcataaataaaaacttggcgattctatatatactcttattatcaaatctagccattgtcttatagatgtgagctactgtaggtgtaccatttgattttctttctaatactatatatttctctcgaagaagttcttgcacatcatctgggaataaaatactactgttgagtaaatcagttattttttttatatcgatattgatggacatttttatagttaaggataataagtatcccaaagtcgataacgacgataacgaagtatttatacttttaggaaatcacaatgactttatcagatcaaaattaacaaaattaaaggagcatgtatttttttctgaatatattgtgactccagatacatatggatctttatgcgtcgaattaaatgggtctagttttcagcacggtggtagatatatagaggtggaggaatttatagatgctggaagacaagttagatggtgttctacatccaatcatatatctgaagatatgcacactgataaatttgtcatttatgatatttatacgtttgattcgttcaagaataaacgattggtatttgtacaggtgcctccatcattaggagatgatagctatttgactaatccgttattgtctccgtattatcgtaattcagtagccagacaaatggtcaatgatatgatttttaatcaagattcatttttaaaatatttattagaacatctgattagaagccactatagagtttctaaacatataacaatagttagatacaaggataccgaagaattaaatctaacgagaatatgttataatagagataagtttaaggcgtttgtattcgcttggtttaacggcgtttcggaaaatgaaaaggtactagatacgtataaaaaggtatctaatttgatataatgaattcagtgactgtatcacacgcgccatatactattacttatcacgatgattgggaaccagtaatgagtcaattggtagagttttataacgaagtagccagttggctgctacgagacgagacgtcgcctattcctgataagttctttatacagttgaaacaaccgcttagaaataaacgagtatgtgtgtgtggtatagatccgtatccgaaagatggaactggtgtaccgttcgaatcaccaaattttacaaaaaaatcaattaaggagatagcttcatctatatctagattaaccggagtaattgattataaaggttataaccttaatataatagacggggttataccctggaattattacttaagttgtaaattaggagaaacaaaaagtcacgcgatctactgggataagatttccaagttactgctgcagcatataactaaacacgttagtgttctttattgtttgggtaaaacagatttctcgaatatacgggcaaagttagaatccccggtaactaccatagtcggatatcatccagcggctagagaccgccaattcgagaaagatagatcatttgaaattatcaacgttttactggaattagacaacaaggcacctataaattgggctcaagggtttatttattaatgctttagtgaaattttaacttgtgttctaaatggatgcaactattagaggtaatgatgttatctttgttcttaagactataggtgtcccgtcagcgtgcagacaaaatgaagatccaagatttgtagaagcatttaaatgcgacgagttagaaagatatattgagaataatccagaatgtacactattcgaaagtcttagggatgaggaagcatactctatagtcagaattttcatggatgtagatttagacgcgtgtctagacgaaatagattatttaacggctattcaagattttattatcgaggtgtcaaactgtgtagctagattcgcgtttacagaatgcggtgccattcatgaaaatgtaataaaatccatgagatctaatttttcattgactaagtctacaaatagagataaaacaagttttcatattatctttttagacacgtataccactatggatacattgatagctatgaaacgaacactattagaattaagtagatcatctgaaaatccactaaccagatcgatagacactgccgtatataggagaaaaacaactcttcgggttgtaggtactaggaaaaatccaaattgcgacactattcatgtaatgcaaccaccgcatgataatatagaagattacctattcacttacgtggatatgaacaacaatagttattacttttctctacaacgacgattggaggatttagttcctgataagttatgggaaccagggtttatttcattcgaagacgctataaaaagagtttcaaaaatattcattaattctataataaactttaatgatctcgatgaaaataattttacaacggtaccactggtcatagattacgtaacaccttgtgcattatgtaaaaaacgatcgcataaacatccgcatcaactatcgttggaaaatggtgctattagaatttacaaaactggtaatccacatagttgtaaagttaaaattgttccgttggatggtaataaactgtttaatattgcacaaagaattttagacactaactctgttttattaaccgaacgaggagaccatatagtttggattaataattcatggaaatttaacagcgaagaacccttgataacaaaactaattctgtcaataagacatcaactacctaaggaatattcaagcgaattactctgtccgaggaaacgaaagactgtagaagctaacatacgagacatgttaatagattcagtggagaccgatacctatccggataaacttccgtttaaaaatggtgtattggacctggtagacggaatgttttactctggagatgatgctaaaaaatatacgtgtactgtatcaaccggatttaaatttgacgatacaaagttcgtcgaagacagtccagaaatggaagagttaatgaatatcattaacgatatccaaccattaacggatgaaaataagaaaaatagagagttgtacgaaaaaactttatctagttgtttatgcggtgctaccaaaggatgtttaacattcttttttggagaaactgcaactggaaagtcgacaaccaaacgtttgttaaagtctgctatcggtgacctgtttgttgagacgggtcaaacaattttaacagatgtattggataaaggacctaatccatttatcgctaacatgcatttgaaaagatctgtattctgtagcgaactacctgattttgcctgtagtggatcaaagaaaattagatctgacaatattaaaaagttgacagaaccttgtgtcattggaagaccgtgtttctccaataaaattaataatagaaaccatgcgacaatcattatcgatactaattacaaacctgtctttgataggatagataacgcattaatgagaagaattgccgtcgtgcgattcagaacacacttttctcaaccttctggtagagaggctgctgaaaataatgacgcgtacgataaagtcaaactattagacgaggggttagatggtaaaatacaaaataatagatatagattcgcatttctatacttgttggtgaaatggtacaaaaaatatcatgttcctattatgaaactatatcctacaccggaagagattccggactttgcattctatctcaaaataggtactctgttagtatctagctctgtaaagcatattccattaatgacggacctctccaaaaagggatatatattgtacgataatgtggttactcttccgttgactactttccaacagaaaatatccaagtattttaattctagactatttggacacgatatagagagcttcatcaatagacataagaaatttgccaatgttagtgatgaatatctgcaatatatattcatagaggatatttcatctccgtaaatatatgctcatatatttatagaagatatcacatatctaaatgaataccggaatcatagatttatttgataatcatgttgatagtataccaactatattacctcatcagttagctactctagattatctagttagaactatcatagatgagaacagaagcgtgttattgttccatattatgggatcaggtaaaacaataatcgctttgttgttcgccttggtagcttccagatttaaaaaggtttacattctagtgcctaatattaacattttgaaaatttttaattataatatgggtgtagctatgaacttgtttaatgacgaattcatagctgagaatatctttattcattccacaacaagtttttattctcttaattataacgataacgtcattaattataacggattatctcgctacaataactctatttttatcgttgatgaggcacataatatctttgggaataatactggagaacttatgaccgtgataaaaaataaaaacaagattccttttttactattgtctggatctcccattactaacacacctaatactctgggtcatattatagatttaatgtccgaagagacgatagattttggtgaaattattagtcgtggtaagaaagtaattcagacacttcttaacgaacgaggtgtgaatgtacttaaggatttgcttaaaggaagaatatcatattacgaaatgcctgataaagatctaccaacgataagatatcacggacgtaagtttctagatactagagtagtatattgtcacatgtctaaacttcaagagagagattatatgattactagacgacagctatgttatcatgaaatgtttgataaaaatatgtataacgtgtcaatggcagtattgggacaacttaatctgatgaataatttagatactttatttcaggaacaggataaggaattgtacccaaatctgaaaataaataatggcgtgttatacggagaagaattggtaacgttaaacattagttccaaatttaaatactttattaatcggatacagacactcaacggaaaacattttatatacttttctaattctacatatggcggattggtaattaaatatatcatgctcagtaatggatattctgaatataatggttctcagggaactaatccacatatgataaacggcaaaccaaaaacatttgctatcgttactagtaaaatgaaatcgtctttagaggatctattagatgtgtataattctcctgaaaacgatgatggcagtcaattgatgtttttgttttcgtcaaacattatgtccgaatcctatactctgaaagaggtaaggcatatttggtttatgactatcccagatactttttctcaatacaaccaaattcttggacgatctattagaaaattctcttacgccgatatttctgaaccagttaatgtatatcttttagccgccgtatattccgatttcaatgacgaagtgacgtcattaaacgattacacacaggatgaattaattaatgttttaccatttgacatcaaaaagctgttgtatctaaaatttaagactaaagaaacgaatagaatatactctattcttcaagagatgtctgaaacgtattctcttccaccacatccatcaattgtaaaagttttattgggagaattggtcagacaatttttttataataattctcgtattaagtataacgataccaagttacttaaaatggttacatcagttataaaaaataaagaagacgctaggaattacatagatgatattgtaaacggtcacttctttgtatcgaataaagtatttgataaatctcttttatacaaatacgaaaacgatattattacagtaccgtttagactttcctacgaaccatttgtttggggagttaactttcgtaaagaatataacgtggtatcttctccataaaactgatgagatatataaagaaataaatgtcgagctttgttaccaatggataccttccagttacattggagccacacgagctgacgttagacataaaaactaatattaggaatgccgtatataagacgtatctccatagagaaattagtggtaaaatggccaagaaaatagaaattcgtgaagacgtggaattacctctcggcgaaatagttaataattctgtagttataaacgttccgtgtgtaataacctacgcgtattatcacgttggggatatagtcagaggaacattaaacatcgaagatgaatcaaatgtaactattcaatgtggagatttaatctgtaaactaagtagagattcgggtactgtatcatttagcgattcaaagtactgcttttttcgaaatggtaatgcgtatgacaatggcagcgaagtcactgccgttctaatggaggctcaacaaggtatcgaatctagttttgtttttctcgcgaatatcgttgactcataaaaaagagaatagcggtaagtataaacacgaatactatggcaataattgcgaatgttttattcccttcgatatatttttgataatatgaaaaacatgtctctctcaaatcggacaaccatctcataaaatagttctcgcgcgctggagaggtagttgctgctcgtataatctccccagaataatatacttgcgtgtcgtcgttcaatttatacggatttctatagttctctgttatataatgcggttttccatcatgattagacgacgacaatagtgttctaaatttagatagttgatcagaatgaatgtttattggcgttggaaaaattatccatacagcgtctgcagagtggttgatagttgttcctagatatgtaaaataatccaacttactaggcagcaaattgtctagataaaatactgaatcaaacggtgcagacgtattggtggatctaatggaatccaattgattaactatcttttgaaaatatacatttttatgatccgatacttgtaagaatatagaaataatgataagtccatcatcgtgtttttttgcctcttcataagaactatattttttcttattccaatgaacaagattaatctctccagagtatttgtacacatctatcaagtgattggatccataatcgtcttcctttccccaatatatacgtagtgatgataacacatattcattggggagaaaccctccacttatatatcctcctttaaaattaatccttactagttttccagtgttctggatagtggttggtttcgactcattataatgtatgtctaacggcttcaatcgcgcgttagaaattgcttttttagtttctatattaataggagatagttgttgcggcatagtaaaaatgaaatgataactgtttaaaaatagctcttagtatgggaattacaatggatgaggaagtgatatttgaaactcctagagaattaatatctattaaacgaataaaagatattccaagatcaaaagacacgcatgtgtttgctgcgtgtataacaagtgacggatatccgttaataggagctagaagaacttcattcgcattccaagcgatattatctcaacaaaattcagattctatctttagagtatccactaaactattacggtttatgtactacaatgaactaagagaaatctttagacggttgagaaaaggttctatcaacgatatcgatcctcactttgaagagttaatattattgggtggtaaactagataaaaaggaatctattaaagattgtttaagaagagaattaaaagaggaaagtgatgaacgtataacagtaaaagaatttggaaatgtaattctaaaacttacaacacgggataaattatttaataaagtatatataagttattgcatggcgtgttttattaatcaatcgttggaggatttatcgcatactagtatttacaatgtagaaattagaaagattaaatcattaaatgattgtattaacgacgataaatacgaatatctgtcttatatttataatatgctagttaatagtaaatgaacttttacagatctagtataattagtcagattattaagtataatagacgactagctaagtctattatttgcgaggatgactctcaaattattacactcacggcattcgttaaccaatgcctatggtgtcataaacgagtatccgtgtccgctattttattaactactgataacaaaatattagtatgtaacagacgagatagttttctctattctgaaataattagaactagaaacatgtctagaaagaaacgattatttctgaattattccaattatttgtccaaacaggaaagaagtatactatcgtcatttttttctctagatccagctactactgataatgatagaatagatgctatttatccgggtggcatacccaaaaggggtgagaatgttccagagtgtttatccagggaaattaaagaagaagttaatatagacaattcttttgtattcatagacactcggttttttattcatggcatcatagaagataccattattaataaattttttgaggtaatcttctttgtcggaagaatatctttaacgagtgatcaaatcattgatacatttaaaagtaatcatgaaatcaaggatctaatatttttagatccgaattcaggtaatggactccaatacgaaattgcaaaatatgctctagatactgcaaaactcaaatgttatggccatagaggatgttattacgaatcattaaaaaaattaactgaggatgattgattagaaaatataaattaatttaccatcgtgtatttttataacgggattgtccggcatatcatgtagatagttaccgtctacatcgtatactcgaccatctacgcctttaaatcctctatttattgacattaatctattagaattggaataccaaatattagtaccctcaattagtttattggtaatattttttttagacgatagatcgatggctcttgaaaccaaggttttccaaccggactcattgtcgatcggtgagaagtctttttcattagcatgaatccattctaatgatgtatgtttaaacactctaaacaattggacaaattcttttgatttgctttgaatgatttcaaataggtcttcgtctacagtaggcataccattagataatctagccattataaagtgcacgtttacatatctacgttctggaggagtaagaacgtgactattgagacgaatggctcttcctactatctgacgaagagacgcctcgttccatgtcatatctaaaatgaagatatcattaattgagaaaaaactaataccctcgcctccactagaagagaatacgcatgttttaatgcattctccgttagtgtttgattcttggttaaactcagccaccgccttgattctagtatcttttgttctagatgagaactctatattagagataccaaagactttgaaatatagtaataagatttctattcctgactgattaacaaatggttcaaagactagacatttaccatgggatgctaatattcccaaacatacatctataaatttgacgcttttctcttttaattcagtaaatagagagatatcagccgcactagcatcccctttcaatagttctccctttttaaaggtatctaatgcggatttagaaaactctctatctcttaatgaatttttaaaatcattatatagtgttgctatctcttgcgcgtattcgcccggatcacgattttgtctttcaggaaagctatcgaacgtaaacgtagtagccatacgtctcagaattctaaatgatgatatacctgtttttatttcagcgagtttagccttttgataaatttcttcttgctttttcgacatattaacgtatcgcattaatactgttttcttagcgaatgatgcagacccttctacgtcatcaaaaatagaaaactcgttattaactatgtacgaacataggcctcctagtttggagactaattctttctcatcaactagacgtttattctcaaatagcgattggtgttgtaaggatcctggtcgtagtaagttaaccaacatggtgaattcttgcacactattaacgataggtgtagccgataaacaaatcatcttatggttttttaatgcgatggtcttagataaaaaattatatactgaacgagtaggacggatcttaccatcttctttgattaatgatttagaaatgaagttatgacattcatcaataatgacgcatattctactcttggaattaatagttttgatattagtaaaaaatttatttctaaaattttgatcatcgtaattaataaaaatacaatccttcgttatctctggagcgtatctgagtatagtgttcatccaaggatcttctatcaaagcctttttcaccaataagataatagcccaattcgtataaatatccttaagatgtttgagaatatatacagtagtcattgttttaccaacacccgtttcatggaacaataaaagagaatgcatactgtctaatcctaagaaaactcttgctacaaaatgttgataatccttgaggcgtactacgtctgttcccatcatttcaacaggcatattagtagttctgcgcaatgcataatcgatataggccgcgtgtgatttactcatttatgagtgataagtaataactatgttttaaaaatcacagcagtagtttaactagtcttctctgatgtttgttttcgatactttttgaatcagaagtcatactagaataaagcaacgagtgaacgtaatagagagcttcgtatactctattcgaaaactctaagaacttattaatgaattccgtatccactggattgtttaaaatactaaattgaacactgttcacatccttccaagaagaagacttagtgacggacttaacatgagacataaataaatccaaattttttttacaaacatcactagccaccataatggcgctatctttcaaccagctatcgcttacgcattttagcagtctaacatttttaaagagactacaatatattctcatagtatcgattacacctctaccgaataaagttggaagtttaataatacaatatttttcgtttacaaaatcaaataatggtcgaaacacgtcgaaggttaacatcttataatcgctaatgtatagattgttttcagtgagatgattattagatttaatagcatctcgttcacgtttgaacagtttattgtgtgcgctgaggtcggcaactacggcgtccgctttagtactcctcccataatactttacgctattaatctttaaaatttcatagactttatctagatcgctttctggtaacatgatatcatgtgtaaaaagttttaacatgtcggtcggcattctatttagatcattaactctagaaatctgaagaaagtaattagctccgtattccagactaggtaatgggcttttacctaaagacaagttaagttctggcaatgtttcataaaatggaagaaggacatgcgttccctcccggatattttttacaatttcatccatttacaactctatagtttgttttcattattattagttattatctcccataatcttggtaatacttaccccttgatcgtaagataccttatacaggtcattacatacaactaccaattgtttttgtacataatagattggatggttgacatccatggtggaataaactactcgaacagatagtttatctttccccctagatacattggccgtaatagttgtcggcctaaagaatatctttggtgtaaagttaaaagttagggttcttgttccattattgctttttgtcagtagttcattataaattctcgagatgggtccgttctctgaatatagaacatcatttccaaatctaacttctagtctagaaataatatcggtcttattcttaaaatctattcccttgatgaagggatcgttaatgaacaaatccttggcctttgattcggctgatctattatctccgttatagacgttacgttgactagtccaaagacttacaggaatagatgtatcgatgatgttgatactatgtgatatgtgagcaaagattgttctcttagtggcatcactatatgttccagtaatggcggaaaactttttagaaatgttatatataaaagaattttttcgtgttccaaacattagcagattagtatgaagataaacactcatattatcaggaacattatcaatttttacatacacatcagcatcttgaatagaaacgataccatcttctggaacctcaacaatctcggcagactccggataaccagtcggtgggccatcactaacaataactagatcatccaacaatctactcacatatgcatctatataatctttttcatcttgtgagtaccctggatacgaaataaatttattatccgtatttccataataaggtttagtataaacagagagcgatgttgccgcatgaacttcagttacagtcgccgttggttggtttatttgacctattactctcctaggtttctctataaacgatggtttaatttgtacattcttaaccatatatccaataaagctcaattcaggaacataaacaaattctttgttgaacgtttcaaagtcgaacgaagagtcacgaataacgatatcggatactggattgaaggttaccgttacggtaatttttgaatcggatagtttaagactgctgaatgtatcttccacatcaaacggagttttaatataaacgtatactgtagatggttctttaatagtgtcattaggagttaggccaatagaaatatcattaagttcactagaatatccagagtgtttcaaagcaattgtattattgatacaattattatataattcttcgccctcaatttcccaaataacaccgttacacgaagagatagatacgtgattaatacatttatatccaacatatggtacgtaaccgaatcttcccatacctttaacttctggaagttccaaactcagaaccaaatgattaagcgcagtaatatactgatccctaatttcgaagctagcgatagcctgattgtctggaccatcgtttgtcataactccggatagagaaatatattgcggcatatacaaagttggaatttgactatcgactgcgaagacattagaccgtttaatagagtcatccccaccgatcaaagaattaatgatagtattattcattttctatttaaaatggaaaaagcttacaataaactccgtagagaaatatctataatttgtgagttttccttaaagtaacagcttccgtaaacgccgtctttatctcttagtaggtttattgtatttatgaccttttccttatcttcatagaatactaaaggcaacaaagaaatttttggttcttctctaagagctacgtgagacttaaccatagaagccaacgaatccctacatattttagaacagaaatacccaacttcaccacccttgaatgtctcaatactaataggtctaaaaaccaaatcttgattacaaaaccaacacttatcaattacactatttgtcttaatagacacatctgccatagatttataatactttggtagtatacaagcgagtgcttcttctttagcgggcttaaagactgctttaggtgctgaaataaccacatctggaaggcttactcgcttagccatttaattacggaactatttttttatacttctaatgagcaagtagaaaacctctcatctacaaaaacatactcgtgtccataatcctctaccatagttacacgttttttagatctcatatgtgctaaaaagttttcccatactaattggttactattatttttcgtataatttttaacagtttgaggttttagatttttagttacagaagtgatatcgaatattttatccaaaaagaatgaataattaattgtcttagaaggagtgttttcttggcaaaagaataccaagtgcttaaatatttctactacttcattaatcttttctgtactcagattcagtttctcatcttttacttgattgattatttcaaagactaacttataatcctttttatttattctctcgttagccttaagaaaactagatacaaaatttgcatctacatcatccgtggatatttgatttttttccatgatatccaagagttccgagataatttctccagaacattgatgagacaataatctccgcaatacatttctcaaatgaataagtttattagacacatggaagtttgactttttttgtacctttgtacatttttgaaatacagactcgcaaaaaatacaatattcatatccttgttcagatactataccgttgtgtctacaaccgctacataatcgtagattcatgttaacactctacgtatctcgtcgtccaatattttatataaaaacattttatttctagacgttgccagaaaatcctgtaatatttttagttttttgggctgtgaataaagtatcgccctaatatggttaccgtcctccgccaatatagtagttaaattatccgcacatgcagaagaacaccgcttaggcggattcagtacaatgttatatttttcgtaccaactcatttaaatatcataatctaaaatagttctgtaatatgtctagcgctaatatattgatcataatcctgtgcataaattaagatacaacaatgtctcgaaatcatcgacatggcttcttccatagttagaagatcgtcgtcaaagttagcaacgtgattcatcaacatttgctgttttgaggcagcaaatactgaaccgtcgccattcaaccattcataaaaaccatcgtctgaatccattgataatttcttgtactggtttttgagagctcgcatcaatctagcatttctagctcccggattgaaaacagaaagaggatcgtacatccagggtccattttctgtaaatagaatcgtataatgtcccttcaagaagatatcagacgatccacaatcaaagaattggtctccgagtttgtaacaaactgcggactttaacctatacatgataccgtttagcatgatttctggtgatacgtcaatcggagtatcatctattagagatctaaagccggtgtaacattctccaccaaacatattcttattctgacgtcgttctacataaaacatcattgctccattaacgataacaggggaatgaacagcactacccatcacattagttcccaatggatcaatgtgtgtaactccagaacatcttccatatcctatgttaggaggagcgaacaccactcttccactattgccatcgaatgccatagaataaatatccttggaattgatagaaatcggactgtcggatgttgtgatcatcttcataggattaacaactatgtatggtgccgcctgaagtttcatatcgtaactgatgccgtttataggtctagccacagaaaccaacgtaggtctaaatccaactatagacaaaatagaagccaatatctgttcttcatctgtcataacttgagagcatccagtatgaataatcttcattagatggggatctaccgcatcatcatcgttacaataaaaaattcccattctaatgttcataattgcttttctaatcatggtatgcatgtttgctctctgaatctctgtggaaattagatctgatacacctgtaatcactatcggattatcctccgtaagacgattaaccaacaacatataattataagactttacttttctaaattcataaagttgctggattaggctataggtgtctccatgtacatacgcgttctcgagcgcaggaagtttaataccgaatagtgccatcagaataggatgaatatagtaattagtttctggttttctataaataaaagacaaatcttgtgaactagacatatcggtaaaatgcatggattggaatcgtgtagtcgacagaagaatatgatgattagatggagagtatattttatctaactctttgagttggtcaccgattctaggactagctcgagaatgaataagtactaaaggatgagtacatttcacagaaacactagcattgttcaatgtgctctttacatgggtaaggagttgaaatagctcgtttctatttgttctgacaatatttagtttattcataatgttaagcatatcctgaatagtaaagttagatgtgtcatacttgttagtagttagatatttagcaattgcattcccatcatttctcaatctcgtactccaatcatgcgtggatgctacttcgtcgatggaaaccatacaatcctttttgataggctgttgagattgattatttcctgcacgtttaggtttggtacgttgatttctagcccctgcggatataaagtcatcgtctacaattttggataatgaattgcatacactacaagacaaagatttatcagaagtgtgaatatgatcttcatctaccaaagaaagagtttgattagtataactagattttagtcctgcgttagatgttaaaaaaacatcgctattgaccacggcttccattatttatattcgtagtttttactcgaaagcgtgattttaatattcaatcttattacttttggaatcgttcaaaacctttgactaattgtagaatttgatctattgccctacgcgtatactcccttgcatcatatacgttcgtcaccagatcgtttgtttcggcctgaagttggtgcatatctctttcaacattcgacatgagatccttaagggccatatcgtctagattttgttgagatgctgctcctggatttggattttgttgtgctgttgtacatactgtaccaccagtaggtgtaggagtacatacagtggccacaataggaggttgaggaggtgtaaccgttggagtagtacaagaaatacttccatccgattgttgtgtacatgtagttgttggtaacgtctgagaaggttgggtagatggcggcgtcgtcgtcttttgatctttattaaatttagagataatatcctgaacagcattgctcggcgtcaacgctggaaggagtgaactcgccggcgcatcagtatctgcagacagccaatcaaaaagattagacatatcagatgatgtattagtttgttgtcgtggttttggtgtaggagccggtgtagctgttggaaccggctgtggagttatatgaatagttggttgtagcggttggataggctgtctgctggcggccatcatattatctctagctagttgttctcgcaactgtctttgataatacgactcttgagactttagtcctatttcaatcgcttcatcctttttcgtatccggatccttttcttcagaataatagattgacgactttggtgtagaggattctgccagcccctgtgagaacttgttaaagaagtccatttaaggctttaaaattgaattgcgattataagattaaatggcagacacagacgatattatcgactatgaatccgatgatctcaccgaatacgaggatgatgaagaagaggaagaagatggagagtcactagaaactagtgatatagatcccaaatcttcttataagattgtagaatcagcatccactcatatagaagatgcgcattccaatcttaaacatatagggaatcatatatctgctcttaaacgacgctatactagacgtataagtctatttgaaatagcgggtataatagcagaaagctataacttgcttcaacgaggaagattacctctagtttcagaattttctgacgaaacgatgaagcaaaatatgctacatgtaattatacaagagatagaggagggttcttgtcctatagtcatcgaaaagaacggagaattgttgtcggtaaacgattttgacaaagatggtctaaaattccatctagactatattatcaaaatttggaaacttcaaaaacgatattagaatttatacgaatatcgttctctaaatgtcacaatcaagtctcgcatgttcagcaatttattgtcgtactttatatcgtgttcattaacgatatcttgcaaaatagtaatgattctatcttccttcgatagatattcttcagagattattgtcttatattctttcttgttatcagatatgaatttgataagactttgaacattattgatacccgtctgtttaattttttctacagatattttagttttggcagattctatcgtatctgtcaatagacatccaacatcgacattcgacgtcaattgtctataaatcaacgtataaattttagaaataacattagcgaattgttgtgcattgatgtcgttattctgaaacagtatgattttaggtagcattttcttaacaaagagaacgtatttattgttactcagttgaacagatgatatatccagattactaacgcatctgattccatataccaaactttcagaagaaatggtgtacaattgtttgtattcattcaatgtctctttttcagaaattagtttagagtcgaatactgcaataattttcaagagatagttttcatcagataagattttatttagtgtagatatgataaaactattgttttgttggagaacttgatacgccgcgttctctgtagtcgacgctctcaaatgggaaacaatctccattatttttttggaatcggatacaatatcttcggtatcttgacgcaatctagtatacatagagttaagagaaattagagtttgtacattaagcaacatgtctctaaatgtggctgcaaacttttccttttccacatcatctagtttattatataccgatttcacaacggcaccagatttaaggaaccagaatgaaaaactctgataactacaatatttcatcatagttacgattttatcatcttctatagttggtgtaatagcgcatacctttttctccaagactggaaccaacgtcataaaaatgtttaaatcaaaatccatatcaacatctgatgcgctaagaccagtctcgcgttcaagattatctttactaatggtgacgaactcatcgtatagaactctaagtttgtccattatttatttacagatttagttgtttaatttatttgtgctcttccagagttgggatagtatttttctaacgtcggtattatattattaggatctacgttcatatgtatcataatattaatcatccacgttttgataaatctatctttagcttctgaaataacgtatttaaacaaaggagaaaaatatttagctacggcatcagacgcaataacattttttgtaaatgtaacgtatttagacgacagatcttcgttaaaaagttttccatctatgtagaatccatcggttgttaacaccattcccgcgtcagattgaataggagtttgaatagtttgttttggaaatagatccttcaataacttatagttgggtgggaaaaaatcgattttatcactagactctttcttttttactatcattacctcatgaactatttcttgaatgagtatatgtattttctttcctatatcggacgcgttcattggaaaatataccatgtcgttaactataagaatatttttatcctcgtttacaaactgaataatatcagatgtagttcgtaaacgaactatatcatcaccagcacaacatctaactatatgatatccactagtttcctttagtcgtttattatcttgttccatattagcagtcattccatcatttaagaaggcgtcaaagataatagggagaaatgacattttggattctgttacaactttaccaaaattaaggatatacggacttactatctttttctcaacgtcgatttgatgaacacacgatgaaaatgtacttcgatgagattgatcatgtagaaaacaacaagggatacaatatttccacatatcatgaaatatattaagaaatcccaccttattatatttccccaaaggatccatgcatgtaaacattatgccgttatcattaataaagacttctttctcatcggatctgtaaaagttgttactgatttttttcattccaggatctagataattaataatgatgggttttctattcttattctttgtattttggcatatcctagaccagtaaacagtttccactttggtaaaatcagcagacttttgaacgctattaaacatggcattaatggcaataactaaaaatgtaaaatatttttctatgttaggaatatggtttttcactttaatagatatatggtttttggccaaaatgatagatatttttttatccgaggatagtaaaatattattagtcgccgtctctataaaaatgaagctagtctcgatatccaattttattctagaattgataggagtcgccaaatgtaccttatacgttatatctcccttgatgcgttccatttgtgtatctatatcggacacaagatctgtaaatagttttacgttattaatcatcacggtatcgccgtcgctagataacgctaatgtaccatccaagtcccaaatggagagatttaactgttcatcgtttagaataaaatgattaccggtcatattaataaagtgttcatcgtatctagataacaacgacttataattaatgtccaagtcttgaactcgctgaatgatcttttttaacccagttagttttagattggtacgaaatatattgttaaactttgattctacagtaatgtccaaatctagttgtggaaatacttccatcaacattgtttcaaacttgataatattattatctacatcttcatacgatccaaattccggaatagatgtatcgcacgctctggccacccagataaccaaaaagtcacacgctccaggatatacattgtataaaaagctatcgttttttagtagtgtttttttctgagtatatacgaagggattaaaaatagtattatcaacgtaactatattccaaattattcttatgagaatagataataatatcgtccttaatatctaacaaatttcctaaatatccctttaattgagtcattcgaagcgtcaatagaatatgtctcttaactatttccggctgttgtatatttaaatgacttcgtaaaaaataatatatgggcgacttctcatctatgtaatcatatggagtgagatatagggctcgttctacctcctgccccttacccacctgtaataccaattgcggacttactatatatcgcatatttatatcgtggggtaaagtgaaaatctactaccgatgatgtaagtcttacaatgttcgaaccagtaccagatcttaatttggaggcctccgtagaactaggggaggtaaatatagatcaaacaacacctatgataaaggaaaatagcggttttatatcccgtagtagacgtctattcgcccatagatctaaggatgatgagagaaaactagcactacgattctttttacaaagactttattttttagatcatagagagattcattatttgttcagatgcgttgacgctgtaaaagacgtcactattaccaaaaaaaataacattatcgtggcgccttatatagcacttttaactatcgcatcaaaaggatgcaaacttacagaaacaatgattgaagcattctttccagaactatataatgaacatagtaagaaatttaaattcaactctcaagtatccatcatccaagaaaaactcggataccagtttggaaactatcacgtttatgattttgaaccgtattactctacagtagctctggctattcgagatgaacattcatctggcatttttaatatccgtcaagagagttatctggtaagttcattatctgaaataacatatagattttatctaattaatctaaaatctgatcttgttcaatggagtgctagtacgggcgctgtaattaatcaaatggtaaatactgtattgattacagtgtatgaaaagttacaactggtcatagaaaatgattcacaatttacatgttcattggctgtggaatcaaaacttccaataaaattacttaaagatagaaatgaattatttacaaaattcatcaacgagttaaaaaagaccagttcattcaagataagcaaacgcgataaggatacgctactaaaatattttacttaggactggagttagaatttatagacgactcatttcgtttatcattattagtattcttcttgttatcttgttcagaaatatacagcaatgctatgcctaatactaaatacattatcatgcttgcaatggctctaacaacgacgaaccaaaatgaatttggtcgtagcttttgttcacaaaaatacataaagaaatgtctacataaatctatggcgccattggctacttgaaatagcgccagtcctcctacagattttaatatagctgtataacatgacatttattcatcatcaaaagagacagagtcaccatctgtcatatttagattttttttcatgtgttcaaagtatcctctactcatttcattataatagtttatcatacttagaattttaggacggatcaatgagtaagacttgactagatcgtcagtagtaatttgtgcatcgtctattctgcatccgcttcgtcgaataatgtatagcatcgctttgagattctccatagctatcaagtctttatacaatgacatggaaatatctgtgaatactttatacttctccaacatcgatgccttaacatcatcgcctactttagcattgaaaatacgttctattgtgtagatggatgtagcaagatttttaaacaacaatgccattttacacgatgattgcctcaagtctccaatcgtttgtttagaacgattagctacagagtccaatgcttggctgactagcatattattatctttagaaattgtattcttcaatgaggcgtttatcatatctgtgatttcgttagtcatattacagtctgactgggttgtaatgttatccaacatatcacctatggatacggtacacgtaccagcatttgtaataatcctatctaagatgttgtatggcattgcgcagaaaatatcttctcctgtaatatttccactctcgataaatctactcagattattcttaaatgccttattctctggagaaaagatatcagtgtccatcatttcattaatagtatacgcagaaaagataccacgagtatcaattctatccaagatacttatcggttccgagtcacagataatggtttcctctccttcgggagatcctgcatagaaatatctaggacaatagtttctatactgtctgtaactctgataatctctaaagtcactaactgataccatgaaattgagaagatcaaacgctgaagtaattaatttttctgcctcgtttttactacaactagttttcatcaatgtagtgacgatgtattgtttagttactcttggtctaatactgatgatagagatattattacttcccataatggatcttctagtagtcaccttaaagcccattgatgcaaatagcagatagataaagtcttggtatgactcctttctaatatagtacggactacctttgtcacccaactttatacccacataagccataacaacctctttaatagccgtttcatgaggtttatcagccatgagcctgagtagttggaagaatctcatgaatcctgtctcagaaagtcctatatgcatgatagatttatctttcctgggaaactctcgtatagtcatagatgaaatactcttcaaagtttctgaaataagattagtaacagtcttacctccgactactctaggtaacaaacaaactctaataggtgttttctctgcggagataatatcagaaaggatagagcaataagtagtattattgtgattataaagaccgaatacataacaggtagaatttataaacatcatgtcctgaaggtttttagacttgtattcctcgtaatccataccgtcccaaaacatggatttggtaactttgatagccgtagatctttgttccttcgccaacaggttaaagaaattaataaagaatttgtggtttctacctatgtctacaaattgcacgtttggaagcgccacggttacattcactgcagcattttgaggatcgcgagtatgaagtacgatgttattgtttactggtatatctggaaagaattctaccagtctaggaataagagattgatatcgcatagaaatacaaaagttcataatctcatcatctaagagcattttgttaccattgtaataaatatccactctgtcatatgtataaatgaagtactgttcaaacatgatgagatgtttatatgttggcatagtagtgagatctacgtttggtaatggcaatgtattaagattaactccataatgtctagcagcatctgcgatgttataagcgttgtcaaagcggggtcgatcttgtgctgttatatattgtctaacacctataagattatcaaaatcttgtctgcttaatacaccgttaacaatttttgccttgaattcttttattggtgcattaataacatccttatagaggatgttaaacaaataagtgttatcaaagttaagatctggatatttcttttctgctagaacatccattgagtcggagccatctggtttaatataaccaccgataaatctagctctgtattctgtatccgtcaatctaatattaagaaggtgttgagtgaaaggtggaagatcgtaaaagctgtgagtattaatgataggattagtttccgaactaatgttaattggggtattaataatatctatatttccagcgttaagtgtaacattaaacagttttaattcacgtgacgtggtatcaattaaataattaatgcccaatttggatatagcagcctgaagctcatcttgtttagttacggatcctaatgagttattaagcaatatatcgaacggatgaacgaaggttgttttaagttggtcacatactttgtaatctagacatagatgcggaagaacggtagaaactatacgaaataaatattcagagtcctctaattgatcaagagtaactattgacttaataggcatcatttatttagtattaaatgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattctccggccactattatcgaacgagtacaacctcatactactattattgacgatactccacctcctacgtttcgtagagagttattgatatcggaacaacgtcaacaacgagaaaaaagatttaatattacagtatcgaaaaatgctgaagcaataatggaatctagatctatgatatcttctatgccaacacaaacaccatccttgggagtagtttatgataaagataaaagaattcagatgttggaggatgaagtggttaatcttagaaatcaacgatctaatacaaaatcatctgataatttagataattttaccagaatactatttggtaagactccgtataaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacgaaaaattatcgtcactaacgtgattattattgtcataaatattatcgagcaagcattgctaaaactcggatttgaagaaatcaaaggactgagtaccgatatcacttcagaaattatcgatgtggagatcggagatgactgcgatgctgtagcatcaaaactaggaatcggtaacagtccggttcttaatattgtattgtttatactcaagatattcgttaaacgaattaaaattatttaatttaatacattcccatatccagacaacaatcgtctggattaatctgttcctgtcgtctcataccggacgacatattaatctttttattagtaggcatctttttagatggtttctttttcccagcattaactgagtcgatacctagaagatcgtgattgatctctccgaccattccacgaacttctaattggccgtctctgacggtaccataaactattttaccagcattagtaacagcttggacaatctgaccatccatcgcattgtacgatgtagtagtaactgttgttctacgtctaggagcaccagaagtatttttggagcccttggatgttgatgtagaagaagacgaggattttgattttggtttacatgtaatacattttgtatcacatgcgccggcagtcacatctgtttgagaattaagattattgttgcctcctttgacggctgcatctccaccgatttgcgctagtagatttttaagctgtggtgtaatcttattaactgtttcgatataatcatcgtaactgcttctaacggctaaattttttttatccgccatttagaagctaaaaatatttttatttatgcagaagatttaactagattatacaatgaactaatatgatccttttccagattatttacaaacttggtattttttggttctggaggaggcgaatttaaattcggacttggatttggattttgtgggttcttgatcttattatacagcgcatataggatggcgacggtaactgctacgcaaataccgatcaacaaaagaataccaatcatttattgacaataacttcactattgatcaagtatgcaatatatcatcttttcactaaataagtagtaataatgattcaacaatgtcgagatatatggacgataataatttagttcatggaaatatcgctatgattggtatgaatgactccgctaactctgtggggcgcgcagtgctttccccacatagaataaattagcattccgactgtgataataataccaagtataaacgccataatactcaatactttccatgtacgagtgggactggtagacttactaaagtcaataaaggcgaagatacacgaaagaatcaaaagaatgattccagcgattagcacgccggaaaaataatttccaatcataagcatcatatccatttaactaataaaaattttaaatcgccgaatgaacaaagtggaatataaaccatataaaaacaatagtttgtactgcaaaaataatatctatttttgttttcgaagatatggtaaaattaaatagtagtacacagcatgttataactaacagcagcaacggctcgtaattacttatcatttactagacgaaaaggtggtgggatattttcttgctcaaataatacgaatatatcacccatccattttatgcgatgtttatatactctaatctttaatagatctatagacgacgggtttaccaacaatatagattttatcgattcatctaatttaaacccttccttaaacgtgaatgatctattatctggcataacgatgactctacccgatgaatcggacaatgtactgggccatgtagaataaattatcaacgaattatcgtctacgaacatttatatcatttgttttaattttagtacgcgaataaatagatataaaatagaaaataacagatattacaaccaatgttatggccgcgcccaaccaggtaggcagttttattttatcttttactacaggttctcctggatgtacgtcaccaacggcggacgtagttctagtacaattagacgtaagttccgcttgggaattttttaacgctaaagagttaacgttaatcgtgcacccaacgtatttacatctagttctttgaacatcttgattataatataaccattttctatctctagattcgtcggtgcactcatgtaaccaacataccctaggtcctaaatatttatctccggaattagattttggataattcgcgcaccaacaatttctatttcctttatgatcgttacaaaagacgtataatgccgtatccccaaaagtaaaataatcaggacgaataattctaataaactcagaacaatatctcgcatccatatgtttggagcaaatatcggaataagtagacatagccggtttccgttttgcacgtaaccattctaaacaattggggtttccaggatcgtttctacaaaatccagtcatgaaatcatcacaatgttctgtcttgtaattattattaaatatttttggacagtgtttggtatttgtcttagaacaacattttgccacgctatcactatcgcccaggagataatccttttttataaaatgacatcgttgcccggatgctatataatcagtagcgtgttttaaatccttaatatattcaggagttacctcgttctgataatagattaatgatccaggacgaaatttgaaagaactacatggttctccatgaattaatacatattgtttagcaaattcaggaactataaaactactacaatgatctatcgacataccatctatcaaacaaaacttgggtttaatttctcccggagatgtttcataatagtacgtataactttcttctgcaaacttaacagctctattatattcaggataattaaaacctaattccatatatttgtctcgtatatctgctattcctggtgctattttgattctattaagagtaacggctgcccccatttttaataatcgtcagtatttaaactgttaaatgttggtatatcaacatttaccttatttcccgcagtataaggtttgttgcaggtatactgttcaggaatggttacatttatactttttctatagtcctgtctttcgatgttcatcacatatgcaaagaacagaataaacaaaataatgtaagaaataatattaaatatctgtgaattcgtaaatacattgattgccataataattacagcagctacaatacatacaatagacattcccacagtgttgccattacctccacgatacatttgagttactaagcaataggtaataactaagctagtaagaggcaatagaaaagatgagataaatatcatcaatatagagattagaggagggctatatagagccaagacgaacaaaatcaaaccgagtaacgttctaacatcattatttttgaagattcccaaataatcattcattcctccataatcgttttgcatcatacctccatctttaggcataaacgattgctgctgttcctctgtaaataaatctttatcaagcactccagcacccgcagagaagtcgtcaagcatattgtaatatcttaaataactcatttatatattaaaaaatgtcactattaaagatggagtataatctttatgccgaactaaaaaaaatgacttgtggtcaacccctaagtctttttaacgaagacggggatttcgtagaagttgaaccgggatcatcctttaagtttctgatacctaagggattttacgcctctccttccgtaaagacgagtctagtatttgaaacattaacaacgaccgataataaaattactagtatcaatccaacaaatgcgccaaagttatatcctcttcaacgcaaagtcgtatctgaagtagtttctaatatgaggaaaatgatcgaatcaaaacgtcctctatacatcactcttcacttggcgtgtggatttggtaagactattaccacgtgttatcttatggctacacacggtagaaaaaccgtcatttgcgtacccaataaaatgttaatacatcaatggaagacacaggtagaggcagtcggattggaacataagatatccatagatggagtaagtagtctattaaaggaactaaagactcaaagtccggatgtattaatagtagtcagtagacatctgacaaacgatgccttttgtaaatatatcaataagcattatgatttgttcatcttggatgaatcacatacgtataatctgatgaacaatacagcagttacaagatttttagcgtattatcctccgatgatgtgttattttttaactgctacacctagaccatctaacagaatttattgtaacagtattattaatattgccaagttatccgatctaaaaaaaactatctatgcagtagatagtttttttgagccatattccacagataatattagacatatgataaaacgattagatggaccatctaataaatatcatatatataccgagaagttattatctgtagacgagcctagaaatcaacttattcttaataccctggtagaagaattcaagtcaggaactattaatcgcattttagttattactaaactacgtgaacatatggtattcttctacaaacgattattagattttttcggatcagaggttgtatttataggagacgcccaaaatagacgtactccagatatggtcaaatcaatcaaggaactaaatagatttatattcgtatccaccttattttattccggtactggtttagatattcctagtttggattctttgttcatttgctcggcagtaatcaacaatatgcaaatagagcaattactagggagggtatgtcgagaaacagaactattagataggacggtatatgtatttcctagcacatccatcaaagaaataaagtacatgataggaaatttcgttcaacgaattattagtctgtctgtagataaactaggatttaaacaaaaaagttatcggaaacatcaagaatccgatcccacttctgtatgtacaacatcctccagagaagaacgtgtattaaatagaatatttaactcgcaaaatcgttaagaagtttaagcgacgatccgcatgctgcgcaggccagtgtattacccctcatagtattaatataatccaatgatacttttgtgatgtcggaaatcttaaccaatttagactgacaggcagaacacgtcatgcaatcatcatcgtcatcgataactgtagtcttgggcttctttttgcggctcttcattccggaacgcacattggtgctatccatttaggtagtaaaaaataagtcagaatatgccctatagcacgatcgtgcaaaacctggtatatcgtctctatctttatcacaatatagtgtatcgacatctttattattattgacctcgtttatcttggaacatggaatgggaacatttttgttatcaacggccacctttgccttaattccagatgttgtaaaattataactaaacagtctatcatcgacacaaatgaaattcttgtttagacgtttgtagtttacgtatgcggctcgttcgcgtctcattttttcagatattgcaggtactataatattaaaaataagaatgaaataacataggattaaaaataaagttatcatgacttctagcgctgatttaactaacttaaaagaattacttagtctgtacaaaagtttgaaattttcagattctgcggctatagaaaagtataattctttggtagaatggggaacatctacttactggaaaataggcgtgcaaaaggtagctaatgtcgagacgtcaatatctgattattatgatgaggtaaaaaataaaccgtttaatattgatccgggctattacattttcttaccggtatattttgggagcgtctttatttattcgaagggtaaaaatatggtagaacttggatctggaaactcttttcaaataccagatgatatgcgaagtgcgtgtaacaaagtattagacagcgataacggaatagactttctgagatttgttttgttaaacaatagatggataatggaagatgctatatcaaaatatcagtctccagttaatatatttaaactagctagtgagtacggattaaacatacccaaatatttagaaattgaaatagaggaagacacattatttgacgacgagttatactctattatagaacgctctttcgatgataaatttccaaaaatatccatatcgtatattaagttgggagaacttaggcggcaagttgtagactttttcaaattctcattcatgtatattgagtccatcaaggtagatcgtataggagataatatttttattcctagcgttataacaaaatcaggaaaaaagatattagtaaaagatgtagaccatttaatacgatccaaggttagagaacatacatttgtaaaagtaaaaaagaaaaacacattttccattttatacgactatgatggaaacggaacagaaactagaggagaagtaataaaacgaattatagacactataggacgagactattatgttaacggaaagtatttctctaaggttggtagtgcaggcttaaagcaattgactaataaattagatattaatgagtgcgcaactgtcgatgagttagttgatgagattaataaatccggaactgtaaaacgaaaaataaaaaaccaatcagcatttgatttaagcagagaatgtttgggatatccagaagcggattttataacgttagttaataacatgcggttcaaaatagaaaattgtaaggttgtaaatttcaatattgaaaatactaattgtttaaataacccgagtattgagactatatatggaaactttaaccagttcgtctcaatctttaatatcgtcaccgatgtcaaaaaaagattattcgagtgaaataatatgcgcctttgatataggtgcaaaaaatcctgccagaactgttttagaagtcaaggataactccgttagggtattggatatatcaaaattagactggagttctgattgggaaaggcacatagctaaagatttgtcacaatatgaatacactacagttcttctagaacgtcagcctagaaggtcgccgtacgtcaaatttatctattttattaaaggctttttatatcatacatcggctgccaaagttatttgcgtctcgcctgtcatgtctggtaattcatatagagatcgaaaaaagagatcggtcgaagcatttcttgattggatggacacattcggattgcgagactccgttccggatagacgcaaattagacgatgtagcggatagtttcaatttggctatgagatacgtattagataaatggaatactaattatacaccttataataggtgtaaatctagaaattacataaaaaaaatgtaataacgttagtaacgccattatggataatctatttacctttctacatgaaatagaagatagatatgccagaactatttttaactttcatctaataagttgcgatgaaataggagatatatatggtcttatgaaagaacgcatttcctcagaggatatgtttgataatatagtgtataataaagatatacatcatgccattaagaaactagtgtattgcgacatccaacttactaaacacattattaatcagaatacgtatccggtatttaacgattcttcacaagtgaaatgttgtcattatttcgatataaactcagataatagcaatattagctctcgtacagtagagatatttgagagggaaaagtcatctcttgtatcatatattaaaactaccaataagaagagaaaggtcaattatggggaaataaagaaaactgtacatggaggcactaatgcaaattacttttccggtaaaaagtctgatgagtatctgagcactacagtcaggtccaacattaatcaaccttggatcaaaaccatttctaagagaatgagagtagatatcattaatcactctatagtaacgcgtggaaaaagctctatattacaaactatagaaattatttttactaatagaacatgtgtgaaaatattcaaggattctactatgcacattattctatccaaggacaaggatgaaaaggggtgtatacacatgattgacaaattattctatgtctattataatttatttctgttgttcgaggatatcatccaaaacgagtactttaaagaagtagctaatgttgtaaaccacgtactcacggctacggcattagatgagaaattattcctaattaagaaaatggctgaacacgatgtttatggagttagcaatttcaaaatagggatgtttaacctgacatttattaagtcgttggatcataccgttttcccctctctgttagatgaggatagcaaaataaagttttttaaggggaaaaagctcaatattgtagcattacgatctctggaggattgtataaattacgtgactaaatccgagaatatgatagaaatgatgaaggaaagatcgactattttaaatagcatagatatagaaacggaatcggtagatcgtctaaaagaattgcttctaaaatgaaaaaaaaacactaattcagaaatggatcaacgactcggatataagtttttggtgcctgatcctaaagccggagttttttatagaccgttacatttccaatatgtatcgtattctaattttatattgcatcgattgcatgaaatcttgaccgtcaagcggccactcttatcgtttaagaataatacagaacgaattatgatagaaattagcaatgttaaagtgactcctccagattactcacctataatcgcgagtattaaaggtaagagttatgacgcattagccacgttcactgtaaatatctttaaagaggtaatgaccaaagagggtatatccatcactaaaataagtagttatgagggaaaagattctcatttgataaaaattccgctactaataggatacgggaataaaaatccacttgatacagccaagtatcttgttcctaatgtcataggtggagtctttatcaataaacaatctgtcgaaaaagtaggaattaatctagtagaaaagattacaacatggccaaaatttagggttgttaagccaaactcattcactttctcgttttcctccgtatcccctcctaatgtattaccgacaagatatcgccattacaagatatctctggatatatcacaattggaagcgttgaatatatcatcgacaaagacatttataacggtcaatattgttttgctgtctcaatatttatctagagtgagtctagaattcattagacgtagtttatcatacgatatgcctccagaagttgtctatctagtaaacgcgataatagatagtgctaaacgaattactgaatctattactgactttaatattgatacatacattaatgacctggtggaagctgaacacattaaacaaaaatctcagttaacgatcaacgagttcaaatatgaaatgctgcataactttttacctcatatgaactatacacccgatcaactaaagggattttatatgatatctttactaagaaagtttctctactgtatctaccacacttctagatatccagatagagattcgatggtttgtcatcgcatcctaacgtacggcaaatattttgagacgttggcacatgatgaattagagaattacataggcaacatccgaaacgatatcatgaacaatcacaagaacagaggcacttacgcggtaaacattcatgtactaacaactcctggacttaatcatgcattttctagtctattgagtggaaagttcaaaaagtcagacggtagttatcgaacacatcctcactattcatggatgcagaatatttctattcctaggagtgttggattttatccggatcaagtaaagatttcaaagatgttttctgtcagaaaataccatccaagtcaatatctttacttttgttcatcggacgttccggaaagaggtcctcaggtaggtttagtatctcaattgtctgtcttgagttccattacaaatatactaacgtctgagtatttggatttggaaaagaaaatttgtgagtatatcagatcatattataaagatgatataagttactttgaaacaggatttccaatcactatagaaaatgctctagtcgcatctcttaatccaaatatgatatgtgattttgtaactgactttagacgtagaaaacggatgggattcttcggtaacttggaggtaggtattactttagttagggatcacatgaatgaaattcgcattaatattggagcgggaagattagtcagaccattcttggttgtggataacggagagctcatgatggatgtgtgtccggagttagaaagcagattagacgacatgacattctctgacattcagaaagagtttccgcatgtcatcgaaatggtagatatagaacaatttacttttagtaacgtatgtgaatcggttcaaaaatttagaatgatgtcaaaggatgaaagaaagcaatacgatttatgtgactttcctgccgaatttagagatggatatgtagcatcttcactagtgggaatcaatcacaattctggacccagagctattcttggatgtgctcaagctaaacaagctatctcttgtctgagctcggatatacgaaataaaatagacaatggaattcatttgatgtatccagagaggccaatcgtgattagtaaggctttagaaacttcaaagattgcggctaattgcttcggccaacatgttactatagcattaatgtcgtacaaaggtatcaatcaagaggatggaattatcatcaaaaaacaatttattcagagaggcggtctcgatattgttacagccaagaaacatcaagtagaaattccgttggaaaactttaataacaaagaaagagataggtctaacgcctattcaaaattagaaagtaatggattagttagactgaatgctttcttggaatccggagacgctatagcacgaaatatctcatcaagaactcttgaagatgattttgctagagataatcagattagctttgatgtttccgaaaaatataccgatatgtacaaatctcgcgttgaacgagtacaagtagaacttactgacaaagttaaggtacgagtattaaccatgaaagaaagaagacccattctaggagacaaatttaccactagaacgagtcaaaagggaacagtcgcgtatatcgcggatgaaacggaacttccatacgatgaaaatggtatcacaccagatgtcattattaattctacatccatcttctctagaaaaactatatctatgttgatagaggttattttaacagccgcatattctgctaagccgtacaacaataagggagaaaaccgacctgtctgttttcctagtagtaacgaaacatccatcgatacatatatgcaattcgctaaacaatgttatgagcattcaaatccgaaattgtctgatgaagaattatcggataaaatcttttgtgaaaagattctctatgatcctgaaacggataagccttatgcatccaaagtattttttggaccaatttattacttgcgtctgaggcatttaactcaggacaaggcaaccgttagatgtagaggtaaaaagacgaagctcattagacaggcgaatgagggacgaaaacgtggaggaggtatcaagttcggagaaatggagagagactgtttaatagcgcatggtgcagccaatactattacagaagttttgaaagattcggaagaagattatcaagatgtgtatgtttgtgaaaattgtggagacatagcagcacaaatcaagggtattaatacatgtcttagatgttcaaaacttaatctctctcctctcttaacaaaaattgataccacgcacgtatctaaagtatttcttactcaaatgaacgccagaggcgtaaaagttaaattagatttcgaacgaaggcctccttcgttttataaaccattagataaagttgatctcaaaccgtcttttctggtgtaatattctagtttggtagtagatacatatcaatatcatcaaattcgagatccgaattataaaatgggcgtggattgttaactatagaatcggacgtctgatattcgaaaatctgtggagttttaggttttggtggaggtgtaactgctacttgggatactgaagtctgatattcagaaagctgggggatgttctggttcgacatccaccgatggtgtcacatcactaatcggttcggtaacgtctgtggacgatggaggcaccacttctacaggttctggttctttatcctcagtcatcaacggagctacttcaatgcgaggaaatgtataatttggtaatggtttctcatgtggatctgaagaagaggtaagatatctactagaaagataccgatcacgttctagttctcttttgtagaacttaactttttctttctccgcatctagttgatattccaacctcttcacgttcgcatgggttacctccgcagtttttacgagcgatttcacgttcagccttcatgcgtcttatagcatgaattcgcttatcgttatcgggtttagcttctgtcaccttagcaattccttttttattaaactctacataatcatatccatttctattgtttgttctaatataaacgagtatagcatcattgctaaatttttcaatagtatcgaaaacagaatatcctaaaccatataatatatattcaggaacactcaaactaaatgtccaggattctcctaaatacgtaaactttaatagtgcgaaatcattcaaaaatctaccacttatagatagatagatagtacataaatgcgtatagtagtctacctatctctttattatgaaaaccggcattacgatcatatatgtcgtgatatacctgtgatccgtttacgttaaaccataaatacatgggtgatcctataaacatgaatttatttctaattctcagagctatagttaattgaccgtgtaatatttgcttacatgcatacttgatacgatcattaataagatttttatcattgctcgttatttcagaatcgtatatataaggagtaccatcgtgattcttaccagatattatacaaaatactatatataaaatatattgacccacgttagtaatcatgtaaatgtttaacgttttaaattttgtattcaatgatccattatcatacgctagcatggtcttatgatattcattctttaaaatataatattgtgttagccattgcattggggctcctaatggagattttttattctcatccattttaggataggctttcataaagtccctaataacttcgtgaataatgtttctatgttttctactgatgcatgtatttgcttcgatttttttatcccatgtttcatctatcatagatttaaacgcagtaatgctcgcaacattaacatcttgaaccgttggtacaattccgttccataaatttataatgttcgccatttatataactcattttttgaatatacttttaattaacaaaagagttaagttactcatatggacgccgtccagtctgaacatcaatctttttagccagagatatcatagccgctcttagagtttcagcgtgattttccaacctaaatagaacttcatcgttgcgtttacaacacttttctatttgttcaaactttgttgttacattagtaatctttttttccaaattagttagccgttgtttgagagtttcctcattgtcgtcttcatcggctttaacaattgcttcgcgtttagcctctggctttttagcagccgttgtagaaaaaaattcagttgctggaattgcaagatcgtcatctccggggaaaagagttccgtccatttaaagtacagattttagaaactgacactctgcgttatttatatttggtacaacacatggattataaatattgatgttaataacatcagaaaatgtaaagtctatacattgttgcatcgtgttaaattttctaatggatctagtattattgggtccaacttctgcctgaaatccaaatatggaagcggatacaaaaccgtttcctggataaaccacacatctccacttttgctttacatcagaaattgtgtcgttgacatcttgaactctcctatctaatgccggtgttccacctatagattttgaatattcgaatgctgcatgagtagcattaaattccttaatattgccataattttcatatattgagtaaccctggataaaaagtaaacacaccgcagccgtagctaccacaataaaaaaaattgatagagagttcatttataatctattagaagctgacaaaatttttttacacgcatcagacaatgctttaataaatagttcaacatctacttttgtcatatcgaaccgatggtatgattctaacctagaattacatccgaaaaagttgactatgttcatagtcattaagtcattaacaaacaacattccagactctggattataagacgatactgtttcgtcacaattacctaccttaatcatgtgattatgaatattggctattagagcaccttctaagaaatctataatatctttgaaacacgatttaaaatcaaaccacgaatatacttctacgaagaaagttagtttacccataggagaaataactataaatggagatctaaatacaaaatccggatctatgatagttttaacattattatattctctattaaatacctccacatctaaaaatgttaattttgaaactatgtcttcgtttattaccgtacctgaactaaacgctataagctctattgtttgagaactctttaaacgatattcttgaaatacatgtaacaaagtttcctttaactcggtcggtttatctaccatagttacagaatttgtatccttatctataatataataatcaaaatcgtataaagttatataattatcgcgttcagattgggatcttttcaaatagactaaaaaccccatttctctagtaagtatcttatgtatatgtttgtaaaatatcttcatggtgggaatatgctctaccgcagttagccattcctcattgacagcggtagatgtattagacaaaactattccaatgtttaacaagggccattttacgagattattaaatccttgtttgataaatgtagccaatgagggttcgagttcaacgacgattgaattctcttcccgcggatgctgcatgatgaacgacgggatgttgttcgattgatttggaattctttttcgactttttgtttatattaaatattttaaaatttatagcggatagcaattcatgtaccacggataatgtagacgcgtattgcgcatcgatatctttattattagataaatttatcaataaatgtgagaagtttgcctcgttaaggtcttccatttaaatattatataaacatttgtgtttgtatcttattcgtcttttatggaatagttttttactagtaaagctgcaattacacactttgtccgtaaaacataaatataaacaccagcttttatcaatcgttccaaaaagtcgacggcggacatttttaacatggcatctattttaaatacacttaggtttttggaaaaaacatcattttataattgtaacgattcaataactaaagaaaagattaagattaaacataagggaatgtcatttgtattttataagccaaagcattctaccgttgttaaatacttgtctggaggaggtatatatcatgatgatttggttgtattggggaaggtaacaattaatgatctaaagatgatgctattttacatggatttatcatatcatggagtgacaagtagtggaacaatttacaaattgggatcgtctatcgatagactttctctaaataggactattgttacaaaagttaataattataattatgatacattttttgacgatgatgattgatcgctattgcacaattttgtttttttactttctaatatagcgtttagattctttttcatgtgcgaatattgatttactaaaatatctatgtttaacttttgttctataacgtccttatcggcggtatcggtacatatacgtaattcaccttcacaaaatacggagtcttcgataataatagccaatcgattattggatctagctgtctgtatcatattcaacatgtttaatatatcctttcgtttcccctttacaggcatcgatcgtagcatattttccgcgtctgagatggaaatgttaaaactacaaaaatgcgtaatgttagcccgtcctaatattggtacgtgtctataagtttggcatagtagaataatagacgtgtttaaatgccttccaaagtttaagaattctattagagtattgcattttgatagtttatcgcctacatcatcaaaaataagtaaaaagtgtgctgattttttatgattttgtgcgacagcaatacatttttctatgttacttttagttcgtatcagattatattctagagattcctgactactaacgaaattaatatgatttggccaaatgtatccatcataatctggattataaacgggtgtaaacaagaatacatgtttatattttttaactagtgtagaaaacagagatagtaaatagatagtttttccagatccagatcctcccgttaaaaccattctaaacggcatttttaataaattttctcttgaaaattgtttttcttggaaacaattcataattatatttacagttactaaattaatttgataataaatcaaaatatggaaaactaaggtcgttagtagggaggagaacaaagaaggcacatcgtgacataaataacatttattatcatgatgacaccagaaaacgacgaagagcagacatctgtgttctccgctactgtttacagagacaaaattcagggaaagaataaacgcaaacgcgtgattggtctatgtattagaatatctatggttatttcactactatctatgattaccatgtccgcgtttctcatagtgcgcctaaatcaatgcatgtctgctaacgaggctgctattactgacgccgctgttgccgttgctgctgcatcatctactcatagaaaggttgcgtctagcactacgcaatatgatcacaaagaaagctgtaatggtttatattaccagggttcttgttatatattacattcagactaccagttattctcggatgctaaagcaaattgcactgcggaatcatcaacactacccaataaatccgatgtcttgactacctggctcattgattatgttaaggatacatggggatctgatggtaatccaattacaaaaactacatccgattatcaagattctgatgtatcacaagaagttagaaagtatttttgtgttaaaacaatgaactaatatttatttttgtacattaataaatgaaatcgcttaatagacaaactgtaagtaggtttaagaagttgtcggtgccggtcgctataatgatgatactctcaaccattattagtggcataggaacatttctgcattacaaagaagaactgatgcctagtgcttgcgccaatggatggatacaatacgataaacattgttatttagatactaacattaaaatgtctacagataatgcggtttatcagtgtcgtaaattacgagccagattgcctagaccggatactagacatctgagagtattgtttagtattttttataaagattattgggtaagtttaaaaaagaccaatgataaatggttagatattaataatgataaagatatagatattagtaaattaacaaattttaaacaactaaacagtacgacggatgctgaagcgtgttatatatacaagtctggaaaactggttaaaacagtatgtaaaagtactcaatctgtactatgtgttaaaaaattctacaagtgacaacaaaaaatgaattaataataagtcgttaacgtacgccgccatggacgccgcgtttgttattactccaatgggtgtgttgactataacagatacattgtatgatgatctcgatatctcaatcatggactttataggaccatacattataggtaacataaaaactgtccaaatagatgtacgggatataaaatattccgacatgcaaaaatgctactttagctataagggtaaaatagttcctcaggattctaatgatttggctagattcaacatttatagcatttgtgccgcatacagatcaaaaaataccatcatcatagcatgcgactatgatatcatgttagatatagaagataaacatcagccattttatctattcccatctattgatgtttttaacgctacaatcatagaagcgtataacctgtatacagctggagattatcatctaatcatcaatccttcagataatctgaaaatgaaattgtcgtttaattcttcattctgcatatcagacggcaatggatggatcataattgatgggaaatgcaatagtaattttttatcataaaagttgtaaagtaaataataaaacaataaatattgaactagtagtacgtatattgagcaatcagaaatgatgctggtacctcttatcacggtgaccgtagttgcgggaacaatattagtatgttatatattatatatttgtaggaaaaagatacgtactgtctataatgacaataaaattatcatgacaaaattaaaaaagataaagagttctaattccagcaaatctagtaaatcaactgatagcgaatcagactgggaggatcactgtagtgctatggaacaaaacaatgacgtagataatatttctaggaatgagatattggacgatgatagcttcgctggtagtttaatatgggataacgaatccaatgttatggcgcctagcacagaacacatttacgatagtgttgctggaagcacgctgctaataaataatgatcgtaatgaacagactatttatcagaacactacagtagtacttaatgaagataccaaacagaatcctaactattcatccaatcctttcgtaaattataataaaaccagtatttgtagcaagtcaaatccgttcattacagaactcaacaataaatttagtgagaataatccgtttagacgagcacatagcgatgattatcttaataagcaagaacaagatcatgaacacgatgatatagaatcattggtgtgattagtttcctttttataaaattgaagtaatatttagtattattgctgccgtcacgttgtacaaatggagatattccctgtattcggcatttctaaaattagcaattttattgctaataatgactgtagatattatatagatacagaacatcaaaaaattatatctgatgagatcaatagacagatggatgaaacggtacttcttaccaacatcttaagcgtagaagttgtaaatgacaatgagatgtaccatcttattcctcatagattatcgacgattatactctgtattagttctgtcggaggatgtgttatctctatagataatgacgtcaatggcaaaaatattctaacctttcccattgatcatgctgtaatcatatccccactgagtaaatgtgtcgtagttagcaagggtcctacaaccatattggttgttaaagcggatatacctagcaaacgattggtaacatcgtttacaaacgacatactgtatgtaaacaatctatcactgattaattattcgccgttgtctgtattcattattagacgagttaccgactatttggatagacacatatgcgatcagatatttgcgaataataagtggtattccattataaccatcgacaataagcagtttcctattccatcaaactgtataggtatgtcctctgccaagtacataaattctagcatcgagcaagatactttaatacatgtttgtaacctcgagcatccattcgacttagtatacaaaaaaatgcagtcgtacaattctgtacctatcaaggaacaaatattgtacggtagaattgataatataaatatgagcattagtatttctgtggattaatagatttctagtatggggatcattaatcatctctaatctctaaatacctcataaaacgaaaaaaaagctattatcaaatactgtacggaatggattcattctcttctctttttatgaaactctgttgtatatctactgataaaactggaagcaaaaaatctgataaaaagaataagaataagatcaaggattattataaaataacaatagttcctggttcctcttccacgtctactagctcgtggtattatacacatgcctagtaatagtctctttgcgttgacggaaagcagactagaaataacaggctaaaatgttcagacaccataatagttcccaacccagataataacagagtaccatcaacacattcctttaaactcaatcccaaacccaaaaccgttaaaatgtatccggccaattgatagtagataatgaggtgtacagcgcatgatgatttacacagtaaccaaaatgaaaatactttagtaattataagaaatatagatggtaacgtcatcatcaacaatccaataatatgccggagagtaaacattgacggataaaacaaaaatgctccgcataactctatcatggcaataacacaaccaaatacttgtaagattcctaaattagtagaaaatacaacggatatcgatgtataagtgatctcgagaaataataagaataaagtaatgcccgtaaagataaacatcaacattgtttggtaatcattaaaccaattagtatgaagttgaactaatttcacagtagattttattccagtattatccccgcatgtataagtacctggtaagatatctttatattccataatcaatgagacatcactatctgataacgaatgaagtctagcactagtatgccatttacttaatattgtcgtcttggaagttttattataagttaaaatatcatggttatccaatttccatctaatatactttgtcggattatctatagtacacggaataatgatggtatcattacatgctgtatactctatggtctttgtagttgttataacaaccaacgtatagaggtatatcaacgatattctaactcttgacattttttatttatttaaaatgatacctttgttatttattttattctattttgctaacggtattgaatggcataagtttgaaacgagtgaagaaataatttctacttacttattagacgacgtattatacacgggtgttaatggggcggtatacacattttcaaataataaactaaacaaaactggtttaactaataataattatataacaacatctataaaagtagaggatgcggaaccaataacggaaatcccaaatgttggaaaatagacggttcagacgacccaaaacatagaggtagaggatacgctccttatcaaaatagcaaagtaacgataatcagtcacaacggatgtgtactatctgacataaacatatcaaaagaaggaattaaacgatggagaagatttgacggaccatgtggttatgatttatacacggcggataacgtaattccaaaagatggtttacgaggagcattcgtcgataaagatggtacttatgacaaagtttacattcttttcactgatactatcggctcaaagagaattgtcaaaattccgtatatagcacaaatgtgcctaaacgacgaaggtggtccatcatcattgtctagtcatagatggtcgacgtttctcaaagtcgaattagaatgtgatatcgacggaagaagttatagacaaattattcattctagaactataaaaacagataatgatacgatactatatgtattcttcgatagtccttattccaagtccgcattatgtacctattctatgaataccattaaacaatctttttctacgtcaaaattggaaggatatacaaagcaattgccgtctccagctcctggtatatgtttaccagctggaaaagttgttccacataccacgtttgaagtcatagaaaaatataatgtactagatgatattataaagcctttatctaaccaacctatcttcgaaggaccgtctggtgttaaatggttcgatataaaggagaaggaaaatgaacatcgggaatatagaatatacttcataaaagaaaattctatatattcgttcgatacaaaatctaaacaaactcgtagctcgcaagtcgatgcgcgactattttcagtaatggtaacttcgaaaccgttatttatagcagatatagggataggagtaggaatgccacaaatgaaaaaaatacttaaaatgtaatcttaatcgagtacaccgcacgacaatgaacaaacataagacagattatgctggttatgcttgctgcgtaatatgcggtctaattgttggaattatttttacagcgacactattaaaagttgtagaacgtaaattagttcatacaccatcaatagataaaacgataaaagatgcatatattagagaagattgtcctactgactggataagctataataataaatgtatccatttatctactgatcgaaaaacctgggaggaaggacgtaatgcatgcaaagctctaaatccaaattcggatctaattaagatagagactccaaacgagttaagttttttaagaagcattagacgcggatattgggtaggagaatccgaaatattaaaccagacaaccccatataattttatagctaaaaatgccacgaagaatggaactaaaaaacggaaatatatttgtagtacaacgaatactcccaaactacatttttatcataccactacttcggttagatgttttagaaaaaaataaatatcgccgtaccgttcttgtttttataaaaataacaattaacaattatcaaattttttctttaatattttacgtggttgaccattcttggtggtaaaataatctcttagtgttggaatggaatgctgtttaatgtttccgcactcatcgtatattttgacgtatgcagtcacatcgtttacgcaatagtcagactgtagttctatcatgcttcctacatcagaaggaggaacagttttaaagtctcttggttttaatctattgccattagttttcatgaaatcctttgttttatccacttcacattttaaataaatgtccactatacattcttctgttaattttactagatcgtcatgggtcatagaatttataggttccgtagtccatggatccaaactagcaaacttcgcgtatacggtatcgcgattagtgtatacaccaactgtatgaaaattaagaaaacagtttaataaatcaacagaaatatttaatcctccgtttgatacagatgcgccatatttatggatttcggattcacacgttgtttgtctgaggtgttcgtctagtgttgcttctacgtaaacttcgattcccatatattctttattgtcagaatcgcataccgatttatcatcatacactgtttgaaaactaaatggtatacacatcaaaataataaataataacgagtacattctgcaatattgttatcgtaattggaaaaatagtgttcgagtgagttggattatgtgagtattggattgtatattttattttatattttatattttgtagtaagaatagaatgctaatgtcaagtttattccaatagatgtcttattaaaaaacatatataataaataacaatggctgaatggcataaaattatcgaggatatctcaaaaaataataagttcgaggatgccgccatcgttgattacaagactacaaagaatgttctagctgctattcctaacagaacatttgccaagattaatcctctcatcactaatcgtaatattctaaaacctcttattggtcagaaatattgtattgtatatactaactctctaatggatgagaacacgtatgctatggagttgcttactgggtacgcccctgtatctccgatcgttatagcgagaactcataccgcacttatatttttgatgggtaagccaacaacatccagacgtgacgtgtatagaacgtgtagagatcacgctacccgtgtacgtgcaactggtaattaaaataaaaagtaatattcatatgtagtgtcaattttaaatgatgatgatgaaatggataatatccatattgacgatgtcaataatgccggtattggcatacagttcatcgatttttagatttcattcagaggatgtggaattatgttatgggcatttgtattttgataggatctataatgtagtaaatataaaatataatccgcatattccatatagatataattttattaatcgcacgttaaccgtagatgaactagacgataatgtcttttttacacatggttattttttaaaacacaaatatggttcacttaatcctagtttgattgtctcattatcaggaaacttaaaatataatgatatacaatgctcagtaaatgtatcgtgtctcattaaaaatttggcaacgagtacatctactatattaacatctaaacataagacttattctctacatcggtccacgtgtattactataataggatacgattctattatatggtataaagatataaatgacatctatgattttactgcaatatgtatgctaatagcgtctacattgatagtgaccatatacgtgtttaaaaaaataaaaatgaactcttaattatgctatgctattagaaatggataaaatcaaaattacggttgattcaaaaattggtaatgttgttaccatatcgtataacttggaaaagataactattgatgtcacacctaaaaagaaaaaagaaaaggatgtattattagcgcaatcagttgctgtcgaagaggcaaaagatgtcaaggtagaagaaaaaaatattatcgatattgaagatgacgatgatatggatgtagaaagcgcataatacgatctataaaaataagtatataaatactttttatttactgtactcttactgtgtagtggtgataccctactcgattatttttttaaaaaaaaatacttattctgattcttctagccatttccgtgttcgttcgaatgccacatcgacgttaaagataggggagtagttgaaatctagttctgcattgttggtacgcacctcaaatgtagtgttggatatcttcaacgtatagttgttgagtagtgatggttttctaaatagaattctcttcatatcattcttgcacgcgtacatttttagcatccatcttggaatcctagatccttgttctattcccaatggtttcatcaatagaagattaaacatatcgtacgaacacgatggagagtaatcgtagcaaaagtaagcatttcctttaatctcagatcccggatactggatatattttgcagccaacacgtgcatccatgcagcatttcctacatatacccggctatgtaccgcgttatcatcgactgtacgatacataatgttaccgtgttgcttacattgctcgtaaaagactttcatcaatttgtctccttctccgtaaattccagtgggtcttaggcaacaagtatacaattttgctccattcatgattacggaattattggctttcataaccagttgctcggccatacgtttactttttgcgtatacatgtcctggtgatatatcataaagggtatgctcatggccgatgaatggatcaccgtgtttattgggtcctattgcttccatgctactagtatagatcaaatacttgattcctaggtccacacaagctgccaatatagtctgtgttccataatagtttactttcatgatttcattatcggtgtattttccaaatacatccactagagcagctgtatgaataatcagatttaccccatctagcgcttctcttaccttatcaaagtcgtttatatcacattgtatatagtttataaccttaactttcgaggttattggttgtggatcttctacaatatctatgactctgatttcttgaacatcatctgcactaattaacagttttactatatacctgcctagaaatccggcaccaccagtaaccgcgtacacggccattgctgccactcataatatcagactacttattctattttactaaataatggctgtttgtataatagaccacgataatatcagaggagttatttactttgaaccagtccatggaaaagataaagttattggattaaaatccggaacgtatagtttgataattcatcgttacggagatattagtcaaggatgtgattccataggcagtccagaaatatttatcggtaacatctttgtaaacagatatggtgtagcatatgtttatttagatacagatgtaaatatatctacaattattggaaaggcgttatctatttcaaaaaatgatcagagattagcgtgtggagttattggtatttcttacataaatgaaaagataatacattttcttacaattaacgagaatggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgactccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaaatataggtgtatggcattagccgaacatatatctgatagtattccaatatctgagctatctagactacgatacaatctatgtaagtatctacgcgggcacactgagagcatagaggatgaatttgattattttgaagacgatgattcgtctacatgttctgccgtaaccgacagggaaacggatgtataattttttttatagcgtgaaggatatgataaaaaatataattgttgtatttatcccattccaatcaccttatatgattctgtaacacaatgaaggagtcttatagatgtatagaggtcagatactggtttgataaactgtttattccacataagtatgtttgactttatggttagacccgcatactttaacaaatcactgaaaattggagttaggtattgacctctcagaatcagttgccgttctggaacattaaatgtattttttatgatatactccaacgcatttatgtgggcatacaacaagtcattactaatggagtattccaagagaagagatttcaacagactgtttatgaactcgaatgccgcctcattgtcgcttatattgatgatgtcgaattctcccaatatcatcaccgatgagtagctcatcttgttatcgggatccaagttttctaaagatgtcattaaaccctcgatcatgaatggatttatcatcatcgtttttatgttggacatgagcttagtccgtttgtccacatctatagacgacgatttctgaattatttcatatatccctctctttaactccaggaacttgtcaggatggtctactttaatatgttctcgtctaagagatgaaaatctttggatggttgcacgcgacttttctctaaaggatcctctcttaaatgaatccatcttatccttggacaagatggacagtctattttccttagatggtttaatatttttgttacccatgatctataaaggtagacctaatcgtctcggatgaccatatatttattttcagttttattatacgcataaattgtaaaaaatatgttaggtttacaaaaatgtctcgtggggcattaatcgtttttgaaggattggacaaatctggaaaaacaacacaatgtatgaacatcatggaatctataccggcaaacacgataaaatatcttaactttcctcagagatccactgtcactggaaagatgatagatgactatctaactcgtaaaaaaacctataatgatcatatagttaatctattattttgtgcaaatagatgggagtttgcatcttttatacaagaacaactagaacagggaattactttaatagttgatagatacgcattctctggagtagcgtatgccgccgctaaaggcgcgtcaatgactctcagtaagagttatgaatctggattgcctaaacccgacttagttatattcttggaatctggtagcaaagaaattaatagaaacgtcggcgaggaaatttatgaagatgttacattccaacaaaaggtattacaagaatataaaaaaatgattgaagaaggagatattcattggcaaattatttcttctgaattcgaggaagatgtaaagaaggagttgattaagaatatagttatagaggctatacacacggttactggaccagtggggcaactgtggatgtaatagtgaaattacattttttataaatggatgaagcatattactctggcaacttggaatcagtactcggatacgtgtccgatatgcataccgaactcgcatcaatatctcaattagttattgccaagatagaaactatagataatgatatattaaacaaggacattgtaaattttatcatgtgtagatcaaacttggataatccatttatctctttcctagatactgtatatactattatagatcaagagatctatcagaccgaattgattaattcattagacgacaatgaaattatcgattgtatagttaacaagtttatgagcttttataaggataacctagaaaatatagtagatgctatcattactctaaaatatataatgaataatccagattttaaaactacgtatgccgaagtactcggttccagaatagcggatatagatattaaacaagtgatacgtgagaatatactacaattgtctaataatatccgcgaacgatatttgtgaaaatattaaaaaaaaatactttttttattaaatgacgtcgcttcgcgaatttagaaaattatgctgtgatatatatcacgcatcaggatataaagaaaaatctaaattaattagagactttataacagatagggatgataaatatttgatcattaagctattgcttcccggattagacgatagaatttataacatgaacgataaacaaattataaaattatatagtataatatttaaacaatctcaggaagatatgctacaagatttaggatacggatatataggagacactattaggactttcttcaaagagaacacagaaatccgtccacgagataaaagcattttaactttagaagaagtggatagttttttaactacgttatcatccgtaactaaagaatcgcatcaaataaaattattgactgatatcgcatccgtttgtacatgtaatgatttaaaatgtgtagtcatgcttattgataaagatctaaaaattaaagcgggccctcggtacgtacttaacgctattagtcctcatgcctatgatgtgtttagaaaatctaataacttgaaagagataatagaaaatgcatctaaacaaaatctagactctatatctatttctgttatgactccaattaatcccatgttagcggaatcgtgtgattctgtcaataaggcgtttaaaaaatttccatcaggaatgtttgcggaagtcaaatacgatggtgaaagagtacaagttcataaaaataataacgagtttgccttctttagtagaaacatgaaaccagtactctctcataaagtggattatctcaaagaatacataccgaaagcatttaaaaaagctacgtctatcgtattggattctgaaattgttcttgtagacgaacataatgtaccgctcccgtttggaagtttaggtatacacaaaaagaaagaatataaaaactctaacatgtgtttgttcgtgtttgactgtttgtactttgatggattcgatatgacggacattccattgtacgaacgaagatcttttctcaaagatgttatggttgaaatacccaatagaatagtattctcagagttgacgaatattagtaacgagtctcagttaactgacgtattggatgatgcactaacgagaaaattagaaggattggtcttaaaagatattaatggagtatacgaaccgggaaagagaagatggttaaaaataaagcgagactatttgaacgagggttccatggcagattctgccgatttagtagtactaggtgcttactatggtaaaggagcaaagggtggtatcatggcagtctttctaatgggttgttacgacgatgaatccggtaaatggaagacggttaccaagtgttcaggacacgatgataatacgttaagggagttgcaagaccaattaaagatgattaaaattaacaaggatcccaaaaaaattccagagtggttagtagttaataaaatctatattcccgattttgtagtagaggatccaaaacaatctcagatatgggaaatttcaggagcagagtttacatcttccaagtcccataccgcaaatggaatatccattagatttcctagatttactaggataagagaggataaaacgtggaaagaatctactcatctaaacgatttagtaaacttgactaaatcttaatagttacatacaaattaaaataacactatttagttggtggtcgccatggatggtgttattgtatactgtctaaacgcgttagtaaaacatggcgaggaaataaatcatataaaaaatgatttcatgattaaaccatgttgtgaaaaagtcaagaacgttcacattggcggacaatctaaaaacaatacagtgattgcagatttgccatatatggataatgcggtatccgatgtatgcaattcactgtataaaaagaatgtatcaagaatatccagatttgctaatttgataaagatagatgacgatgacaagactcctactggtgtatataattattttaaacctaaagatgccattcctgttattatatccataggaaaggatagagatgtttgtgaactattaatctcatctgataaagcgtgtgcgtgtatagagttaaattcatataaagtagccattcttcccatggatgtttccttttttaccaaaggaaatgcatcattgattattctcctgtttgatttctctatcgatgcggcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcaatagctgataatagaacttacgcaaatattagcaaaaatatattagacaatactacaattaacgatgagtgtagatgctgttattttgaaccacagattaggattcttgatagagatgagatgctcaatggatcatcgtgtgatatgaacagacattgtattatgatgaatttacctgatgtaggcgaatttggatctagtatgttggggaaatatgaacctgacatgattaagattgctctttcggtggctggtaaaaaattgaaaataaatacaaaggttcttgagggttgtgttaaattgaaagcgagaaataatcataaataagataaacgccgccaccatgtctgataacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtagaggaacttctcctgctagaatggctggcaatggcggtgatgctgctcttgctttgctgctgcttgacagattgaaccagcttgagagcaagatgtctggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaggcgcgcctttttatactagtatttggaaagttttataggtagttgatagaacaaaatacataattttgtaaaaataaatcactttttatactaatatgacacgattaccaatacttttgttactaatatcattagtatacgctacaccttttcctcagacatctaaaaaaataggtgatgatgcaactttatcatgtaatcgaaataatacaaatgactacgttgttatgagtgcttggtataaggagcccaattccattattcttttagctgctaaaagcgacgtcttgtattttgataattataccaaggataaaatatcttacgactctccatacgatgatctagttacaactatcacaattaaatcattgactgctagagatgccggtacttatgtatgtgcattctttatgacatcgcctacaaatgacactgataaagtagattatgaagaatactccacagagttgattgtaaatacagatagtgaatcgactatagacataatactatctggatctacacattcaccggaaactagttctgagaaacctgattatatagataattctaattgctcgtcggtattcgaaatcgcgactccggaaccaattactgataatgtagaagatcatacagacaccgtcacatacactagtgatagcattaatacagtaagtgcatcatctggagaatccacaacagacgagactccggaaccaattactgataaagaagaagatcatacagttacagacactgtctcatacactacagtaagtacatcatctggaattgtcactactaaatcaaccaccgatgatgcggatctttatgatacgtacaatgataatgatacagtaccatcaactactgtaggcggtagtacaacctctattagcaattataaaaccaaggactttgtagaaatatttggtattaccgcattaattatattgtcggccgtggcaatattctgtattacatattatatatataataaacgttcacgtaaatacaaaacagagaacaaagtctagatttttgacttacataaatgtctgggatagtaaaatctatcatattgagcgggccatctggtttaggaaagacagccatagccaaaagactatgggaatatatttggatttgtggtgtcccataccactagatttcctcgtcctatggaacgagaaggtgtcgattaccattacgttaacagagaggccatctggaagggaatagccgccggaacatactgagtttttaggaaatatttacggaacttctaaaactgctgtgaatacagcggctattaataatcgtatttgtgtgatggatctaaacatcgacggtgttagaagttttaaaaatacttacctaatgccttactcggtgtatataagacctacctctcttaaaatggttgagaccaagcttcgttgtagaaacactgaagcggatgatgagattcatcgtcgtgtgatgttggcaaaaactgacatggatgaggcaggtgaagccggtctattcgacactattatcattgaagatgatgtgaatttagcatatagtaagttaattcagatactacaggaccgtattagaatgtattttaacactaattagagacttaagacttaaaacttgataattaataatataactcgtttttatatgtggctatttcaacgtctaatgtattagttaaatattaaaacttaccacgtaaaacttaaaatttaaaatgatatttcattgacagatagatcacacattatgaactttcaaggacttgtgttaactgacaattgcaaaaatcaatgggtcgttggaccattaataggaaaaggtggatttggtagtatttatactactaatgacaataattatgtagtaaaaatagagcccaaagctaacggatcattatttaccgaacaggcattttatactagagtacttaaaccatccgttatcgaagaatggaaaaaatctcacaatataaagcacgtaggtcttatcacgtgcaaggcatttggtctatacaaatccattaatgtggaatatcgattcttggtaattaatagattaggtgcagatctagatgcggtgatcagagccaataataatagattaccaaaaaggtcggtgatgttgatcggaatcgaaatcttaaataccatacaatttatgcacgagcaaggatattctcacggagatattaaagcgagtaatatagtcttggatcaaatagataagaataaattatatctagtggattacggattggtttctaaattcatgtctaatggcgaacatgttccatttataagaaatccaaataaaatggataacggtactctagaatttacacctatagattcgcataaaggatacgttgtatctagacgtggagatctagaaacacttggatattgtatgattagatggttgggaggtatcttgccatggactaagatatctgaaacaaagaattgtgcattagtaagtgccacaaaacagaaatatgttaacaatactgcgactttgttaatgaccagtttgcaatatgcacctagagaattgctgcaatatattaccatggtaaactctttgacatattttgaggaacccaattacgacaagtttcggcacatattaatgcagggtgtatattattaagtgtggtgtttggttgatgtaaaatttttgtcgataaaaattaaaaaataacttaatttattattgatctcgtgtgtacaaccgaaatcatggcgatgttttacgcacacgctctcggtgggtacgacgagaatcttcatgcctttcctggaatatcatcgactgttgccaatgatgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcacatatcgccacaaaaactactatgctctaagcggaatcgggtacgagagtctagacttgtgtttggaaggagtagggattcatcatcacgtacttgaaacagaaaacgctgtatatggaaaagttcaacatgattattctactatcaaagagaaggccaaagaaatgaatgcactcagttcaggacctatcatcgattaccacgtctggataggagattgtatctgtcaagttactgctgtggacgtacatggaaaggaaattatgagaatgagattcaaaaagggtgcggtgcttccgatcccaaatctggtaaaagttaaacttggggagaatgatacagaaaatctttcttctactatatcggcggcaccatcgaggtaaccacctctctggaagacagtgtgaatcatgtactcatgaaacgtttggaatctatacgccatatgtggtctgttgtatatgatcattttgatattgtgaatggtaaagaatgctgttatgtgcatacgcatttgtctaatcaaaatcttataccgagtactgtaaaaacaaatttgtacatgaagactatgggatcatgcattcaaatgagtatcttagcgaactgaaggaatcaggtggatggagtcccagaccagaaatgcaggaatttgaatatccagatggagtggaagacactgaatcaattgagagattggtagaggagttcttcaatagatcagaacttcaggctggtaaatctattaatgttaaacatacatctgtttcagctaagcaactaagaacacgtatactctcatcttttgccaacacagagggtggatatttgttcattggagttgataataatacacacaaagtatttggattcacggtgggttacgactacctcagactgatagagaatgatatagaaaagcatatcaaaagactttgtgttgtgtatttctgtgagaagaaagaggacatcaagtacacgtgtcgattcatcaaggtatataaacctggggatgaggctacctcgacatacgtgtgcgctatcaaagtggaaagatgctgttgtgctgtgtttgcagattggccagaatcatggtatcaagaagtattctccagatgaataggtgtcacatataaaattttaattaatgtaactatagagaacaaataataggttgtaatatcatatagacaataactaacaattaattagtaactgttatctctttttaactaactaactatacctattaatacatcgtaattatagttcttaacatctattaatcattgattcgcttctttaattttttataaaccaacattgttaattgaaaagggataacatgttacagaatataaattatatatggattttttttaaaaaggaaatacttgactggagtgtatatttatctcttcattatatagcacgcgtgttttccaatttttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcaggttatcatcatccattaatataaaagaatatgtatatcaattagttaataatgatcatccagataataggataagactaatgcttgaaaatggacgtagaacaagacattttttgtcctatatatcagatacagttaatatctatatatgtattttatatagatgccgaagacagttacggttgtacattattacatagatgtatatatcactataagaaatcagaatcagaatcagaatcatacaatgaattaattaagatattgttaaataatggatcagatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaataaattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatttacgacgctgtcagttataatgcgtataatacgttggtctatctattaaacagaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaacataaacagcataatgcagatttattgaaaatgtgtataaaatatactgcgtgtatgaccgattatgatactcttatagatgtacaatcactacagcaatataaatggtatattttaagatgtttcgatgaaatagatatcatgaagagatgttatataaaaaataaaactgtattccaattagttttttgtatcaaagacattaatactttaatgagatacggtaaacatccttctttcgtgaagtgcactagtctcgacgtatacggaagtcgtgtacgtaatatcatagcatctattagatatcgtcagagattaattagtctattatccaagaagctggatcctggagataaatggtcgtgttttcctaacgaaataaaatataacgataacgaactgtccacatatctaaaaatcttataaacactattaaaatataaaatcacactacatcattgtttccttttagtgctcgacagtgtatactatttttaacgctcataaataaaaatgaaaacgatttccgttgttacgttgttatgcgtactacctgctgttgtttattcaacatgtactgtacccactatgaataacgctaaattaacgtctaccgaaacatcgtttaataataaccagaaagttacgtttacatgtgatcagggatatcattcttcggatccaaatgctgtctgcgaaacagataaatggaaatacgaaaatccatgcaaaaaaatgtgcacagtttctgattacatctctgaactatataataaaccgctatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatataactatcaactgtgatgttggatatgaggttattggtgcttcgtacataagttgtacagctaattcttggaatgttattccatcatgtcaacaaaaatgtgatataccgtctctatctaatggattaatttccggatctacattttctatcggtggcgttatacatcttagttgtaaaagtggttttatactaacgggatctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtgaatataaatccgttaaaataatgaataattaataattaataatttaataacaaacaagtatcaaaagattaaagacttatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccatgagatatattataattctcgcagttttgttcattaatagtatacacgctaaaataactagttataagtttgaatccgtcaattttgattccaaaattgaatggactggggatggtctatacaatatatcccttaaaaattatggcatcaagacgtggcaaacaatgtatacaaatgtaccagaaggaacatacgacatatccgcatttccaaagaatgatttcgtatctttctgggttaaatttgaacaaggcgattataaagtggaagagtattgtacgggaccaccgactgtaacattaactgaatacgacgaccatccgtatgctactagaggtagcaaaaagattcctatttacaaacgcggtgacatgtgtgatatctacttgttgtatacggctaacttcacattcggagattctaaagaaccagtaccatatgatatcgatgactacgattgcacgtctacaggttgcagcatagactttgtcacaacagaaaaagtgtgcgtgacagcacagggagccacagaagggtttctcgaaaaaattactccatggagttcgaaagtatgtctgacacctaaaaagagtgtatatacatgcgcaattagatccaaagaagatgttcccaatttcaaggacaaaatggccagagttatcaagagaaaatttaactaaatttctcggtagcacatcaaatgatgttaccacttttcttagcatgcttaacttgactaaatattcataactaatttttattaatgatacaaaaacgaaataaaactgcatattatacactggttaacgcccttataggctctaaccattttcaagatgaggtccctgattatagtccttctgttcccctctatcatctactccatgtctattagacgatgtgagaagactgaagaggaaacatggggattgaaaatagggttgtgtataattgccaaagatttctatcccgaaagaactgattgcagtgttcatctcccaactgcaagtgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtctccccgctataaaggcattatcaacttctccttattgattgaaaatgaaaatataaatagtttttatgtatagcagtattaccctatagttttattgcttactactaacatggatacagatacagatgttacaaatgtagaagatatcatgaatgaaatagatagagagaaagaagaaatactaaaaaatgtagaaattgaaaataataaaaacattaacaagaatcatcccaatgaatatattagagaagcactcgttattaatacaagtagtaatagtgattccattgataaagaagttatagaatgtatcagtcacgatgtaggaatatagatcatatctactaatttttataatcaatacaaaacataaaaaacaactcgttattacatagcaggcatggaatccttcaagtattgttttgataacgatggcaagaaatggattatcggaaatactttatattctggtaattcaatactctataaggtcagaaaaaatttcactagttcgttctacaattacgtaatgaagatagatcacaaatcacacaagccattgttgtctgaaatacgattctatatatctgtattggatcctttgactatcgacaactggacacgggaacgtggtataaagtatttggctattccagatctgtatggaattggagaaaccgatgattatatgttcttcgttataaagaatttgggaagagtattcgccccaaaggatactgaatcagtcttcgaagcatgcgtcactatgataaacacgttagagtttatacactctcgaggatttacccatggaaaaatagaaccgaggaatatactgattagaaataaacgtctttcactaattgactattctagaactaacaaactatacaagagtggaaactcacatatagattacaacgaggacatgataacttcaggaaatatcaattatatgtgtgtagacaatcatcttggagcaacagtttcaaaacgaggagatttagaaatgttgggatattgcatgatagaatggttcggtggcaaacttccatggaaaaacgaaagtagtataaaagtaataaaacaaaaaaaagaatataaaaaatttatagctactttctttgaggactgttttcctgaaggaaatgaacctctggaattagttagatatatagaattagtatacacgttagattattctcaaactcctaattatgacagactacgtagactgtttatacaagattgaaatattctttttttatagagtgtggtagtgttacggatatctaatattaatattagactatctctatcgcgctacacgaccaatatcgattactatggatatcttctatgaaaggagagaatgtattcatttctccagcgtcaatctcgtcagtattgacaatactgtattatggagctaatggatccactgctgaacagctatcaaaatatgtagaaaaggaggagaacacggataaggttagcgctcagaatatctcattcaaatccatgaataaagtatatgggcgatattctgccgtgtttaaagattcctttttgagaaaaattggcgataagtttcaaactgttgacttcactgattgtcgcactatagatgcaatcaacaagtgtgtagatatctttactgaggggaaaatcaatccactattggatgaacaattgtctcctagcaattagtgccgtatactttaaagcaaaatggttgacgccattcgaaaaggaatttaccagtgattatcccttttacgtatcaccaacggaaatggtagacgtaagtatgatgtctatgtacggcgagctatttaatcacgcatctgtaaaagaatcattcggtaacttttcaatcatagaactgccatatgttggagatactagtatgatggtcattcttccagacaagattgatggattagaatccatagaacaaaatctaacagatacaaattttaagaaatggtgtaactctctggaagctacgtttatcgatgttcacattcccaagtttaaggtaacaggctcgtataatctggtggatactctagtaaagtcaggactgacagaggtgttcggttcaactggagattatagcaatatgtgtaatttagatgtgagtgtcgacgctatgatccacaaaacgtatatagatgtcaatgaagagtatacagaagcagctgcagcaacttgtgcactggtgtcagactgtgcatcaacaattacaaatgagttctgtgtagatcatccgttcatctatgtgattaggcatgttgatggaaaaatttttttcgttggtagatattgctctccgacaactaattgttaaccattttttttaaaaaaatagaaaaaacatgtggtattagtgcaggtcgttattcttccaattgcaattggtaagatgacggccaactttagtacccacgtcttttcaccacagcactgtggatgtgacagactgaccagtattgatgacgtcaaacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgtagaaatcatccgttgttatataccggatgagcaaaaaaccatcatcggactttgtgcatatgctgctacttactggggaggtgaagaccatcccactagtaacagtctgaacgcattgtttgtgatgcttgagatgctaaattacgtggattataacatcatattccggcgtatgaattgatgagttgtacatcttgacattttcttctttcttctcttctccctttcccagaaacaaactttttttacccactataaaataaaatgagtatactacctgttatatttctttctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcgggcatcgacgccttagaaataagagacttaaacaacggacacctggaattattaccatagaagatgttagaaaaaatgatgctggttattatacatgtgttttagaatatatatacggtggcaaaacatataacgtaaccagaattgtaaaattagaggtacgggataaaataataccttctactatgcaattaccagaaggtgttgtaacttcaataggtagtaatttgactattgcgtgtagagtatcgttgagacctcccacaacggatgcagacgtcttttggataagtaatggtatgtattacgaagaagatgatggggacggagacggtagaataagtgtagcaaataaaatctatatgaccgataagagacgtgttattacatcccggttaaacattaatcctgtcaaggaagaagatgctacaacgtttacgtgtatggcgtttactattcctagcatcagcaaaacagttactgttagtataacgtgaatgtatgttgttacatttccatgtcaattgagtttataagaatttttatacattatcttccaacaaacaattgacgaacgtattgctatgattaactcccacgatactatgcatattattaatcattaacttgcagactatacctagtgctattttgacatactcatgttcttgtgtaattgcggtatctatattattaaagtacgtaaatctagctatagttttattatttaattttagataatataccgtctccttatttttaaaaattgccacatcctttattaaatcatgaatgggaatttctatgtcatcgttaatatattgtgaacaacaagagcagatatctataggaaagggtggaatgcgatacattgatctatgtagttttaaaacacacgcgaactttgaagaatttatataaatcattccatcgatacatccttctatgttgacatgtatatatccaggaattcttttattaatgtcaggaaatgtataaactaaaacattgcccgaaagcggtgcctctatctgcgttatatccgttcttaacttacaaaatgtaaccaatacctttgcatgacttgttttgttcggcaacgttagtttaaacttgacgaatggattaattacaatagcatgatccgcgcatctattaagtttttttactttaacgcccttgtatgtttttacagagactttatctaaatttctagtgcttgtatgtgttataaatataacgggatatagaactgaatcacctaccttagatacccaattacattttatcagatccagataataaacaaattttgtcgccctaactaattctatattgttatatattttacaattggttatgatatcatgtaataacttggagtctaacgcgcatcgtcgtacgtttatacaattgtgatttagtgtagtatatctacacatgtatttttccgcactatagtattctggactagtgataaaactatcgttatatctgtcttcaatgaactcatcgagatattgctctctgtcatattcatacacctgcataaactttctagacatcttacaatccgtgttattttaggatcatatttacatatttacgggtatatcaaagatgttagattagttaatgggaatcgtctataataatgaatattaaacaattatatgaggacttttaccacaaagcatcataaaaatgagtcgtcgtctgatttatgttttaaatatcaaccgcgaatcaactcataaaatacaagagaatgaaatatatacatattttagtcattgcaatatagaccatacttctacagaacttgattttgtagttaaaaactatgatctaaacagacgacaacctgtaactgggtatactgcactacactgctatttgtataataattactttacaaacgatgtactgaagatattattaaatcatggagtggatgtaacgatgaaaaccagtagcggacgtatgcctgtttatatattgcttactagatgttgtaatatttcacatgatgtagtgatagatatgatagacaaagataaaaaccacttatcgcatagagactattccaacctactactagagtatataaaatctcgttacatgttattgaaggaagaggatatcgatgagaacatagtatccactttattagataagggaatcgatcctaactttaaacaagacggatatacagcgttacattattattatttgtgtctcgcacacgtttataaaccaggtgagtgtagaaaaccgataacgataaaaaaggccaagcgaattatttctttgtttatacaacatggagctaatctaaacgcgttagataattgtggtaatacaccattccatttgtatcttagtattgaaatgtgtaataatattcatatgactaaaatgctgttgacttttaatccgaatttcaaaatatgtaataatcatggattaacgcctatactatgttatataacttccgactacatacaacacgatattcttgttatgttaatacatcactatgaaacaaatgttggagaaatgccgatagatgagcgtcgtataatcgtattcgagtttatcaaaacatattctacacgtccggcagattcgataacttatttgatgaataggtttaaaaatatagatatttatacccgctatgaaggaaagacattattacacgtagcatgtgaatataataatacacacgtaatagattatcttatacgtatcaacggagatataaatgcgttaaccgacaataacaaacacgctacacaactcattatagataacaaagaaaattccccatataccattaattgtttactgtatatacttagatatattgtagataagaatgtgataagatcgttggtggatcaacttccatctctacctatcttcgatataaaatcatttgagaaattcatatcctactgtatacttttagatgacacattttacaatagacacgttaggaatcgcaattctaaaacgtatcgatacgcattttcaaaatacatgtcgtttgataaatacgatggtataataactaaatgtcataaagaaacaatattgctcaaactatccactgttctagacactacactatatgcagttttaagatgccataattcgaaaaagttaagaagatacctcaacgagttaaaaaaatataataacgataagtcctttaaaatatattctaatattatgaatgagagataccttaatgtatattataaagatatgtacgtgtcaaaggtatatgataaactatttcctgttttcacagataaaaattgtctactaacattactaccttcagaaattatatacgaaatattatacatgctgacaattaacgatctttataatatatcgtatccacctaccaaagtatagttgtatttttctcatgcgatgtgtgtaaaaaaactgatattatataaatattttagtgccgtataatgaagatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggagaggctagaaaaaaatagacggcgacaggtttctaataaacgtgttaaacatggtgatttatggatagccaactatacatctaaattcagtaaccgtaggtatttgtgtaccgtaactacaaagaatggtgactgtgttcagggtatagttagatctcatattaaaaaacctccttcatgcattccaaaaacatatgaactaggtactcatgataagtatggcatagacttatactgtggaattctttacgcaaaacattataataatataacttggtataaagataataaggaaattaatatcgacgatattaagtattcacaaacgggaaagaaattaattattcataatccagagttagaagatagtggaagatacaactgttacgttcattacgacgacgttagaatcaagatgtaaaatacttacggttataccgtcgcaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtatcaccgaggcgaccttgtactttgaaaatgttactgaagaatatataggtaatacatataaatgtcgtggacacaactattattttgaaaaaacccttacaactacagtagtattggagtaaatacacaatgcatttttatatacattactgaataattattattattatttatatcgtatttgtgctataacgcgactatctaggtatttgtatctcaccgatagagaacatataaatgtagactctattaaacagttgtgtaaaatatcagatcctaatagatgtggatgtacggctttagaaatgagttcattaaaatatgtgatatcaacggaacatatttatataattatactattgctgttagtataattattgattccacggaagaactaccaacagttactccaattacaacaacatataattatactatcgatgatagcactactgaagaactacaagtgactcctcatatggatctccatcgatgatacatgtattaaaatactttccgaataagtcttttaaatattgtattaattatgaaaaactatgctatgcgagtatgatacgatactagattttatctctagcgagagatgtcgttagaatcatttatcaacgaatatcgataacatgtgtcatttatacgttaaagtctgtccgtcttctctattgtttagactgtttgtagaatgctgtgatataaacaaactagtagacacaaatatttaactcatgatgaagttgagaatgatatgctttagctaatataaaaatatattaatccactatatattctagacttgatttaaaaccgataaactactactacgtactgtataagttaggagcagaccctaattatgtagatgatagaggtaatacttctgcatctatatgtccacttatgagaaaacgtcatttaataagatgcatcgtgaaaagaaatttattaaagagttggtaaaatatgaaaccgaaagtaaataatataggaaatacacctctacataactacgtatctcaatatgatatcactctcattcctcatccacaacccattaaaaaatggaaattaaagccctctattagcataaacggctacaggtctacctttacaatggcctttccttgtgcccagttcagaccctgtcattgccacgctactaaggactccctgaataccgtggccgacgtcagacattgtctgactgaatacatcctgtgggtttctcatagatggacccatagagaaagcgcagggtctctctacaggcttctcatctctttcagaactgatgcaacggagctctttggtggtgagttgaaggattcacttccgtggagatcattaaatgactccatgaaaaccgccgaagaacttcgtgcaatcattggactttgtactcaatcagctatcgtctctggaagagtcttcaacgataagtatatcgacatactacttatgctgcgaaagattctgaacgagaacgactatctcaccctcttggatcatatccgcactgctaaatactaaatctccttcatgctctctcactacactttttatcatcttatgaggaataattagcaccagaatagctatggattgcacatgtattctatgtcgtctactggatgaagatgtgacgtacaaaaaaataaaactagaaattgaaacgtgtcacaacttatcaaaacatatagatagacgaggaaacaatgcgctacattgttacgtctccaataaatgcgatacagacattaagattgttcgactgttactctctcgcggagtcgagagactttgtagaaacaacgaaggattaactccgctaggagcatacagtaagcatagatacgtaaaatctcagattgtgcatctactgatatccagctattcgaattcctctaacgaactcaagtcgaatataaatgatttcgacttacgtctgctaaaatacctaattgtggataaacggatacgtccgtccaagaatacgaattatgcaatcaatggtctcggattggtggatatatacgtaacgacgcctaatccgagaccagaagtattgctatggcttcttaaatcagaatgttacagcaccggttacgtatttcgtacctgtatgtacaacagtgatatgtgtaagaactctcttcattactatatatcgtctcatagagaatctctatccaaggatgtaattaaatgtttgatcgataacaatgtttccatccaatactactggtcttgctcaaccatagatatagagattattaataaaggatgtggacacgtgtagagtatacgacgtcagccctatattagaggcgtattatctaaacaagcgatttagagtaaccccatataatgtagacatggaaatcgttaatcttcttattgagagacgtcatactcttgtcgacgtaatgcgtagtattacttcgtacgattccagagaatataaccactacatcatcgataacattctaaagagatttagacaacaggatgaatccatcgtacaagccatactgataaactacttacattacggcgatatggtaagtatacctatcattcaatgcatgttggataagacgacggacaacaactttgttaataataatctcgtcgatgtaaacgtcgtaaggtttatcgtggaaaatatggacacgcggctgtaaatcacatatctaacaatggccgtctatgtatgtacggtctgatattatcgagatttaataattgcgggtatcactgttatgaagatgtatttgatatactaagcaagtacatggatgatatagatatgatcgataactctactatattacgcggtcgatgtcaataatatacaatttgcaaagcggttattggaatatggagcgagtgtcacgctcgataatcaatacggccatccagaaaagcagttaccaaagagaagctagttgatttattactgagttaccatcccactctagagactatgattgacgcatttaatagagatatacgctatctatatcctgaaccattattcgcctgtatcagatacgccttaatcctagatgatgattttccttctaaagtaagtatgatatcgccggtcgtcataaggaactaaagcgctatagagcagacattaatagaatgaagaatgcctacatatcaggcgtctccatgtttgatatattatttaaacgaagcaaacgccacagattgagatacgcaaagaacaatgagaggatcgactccattaaataatttatcatggagtgataatgtcctgtttccatggcatattacaaaatcgattccgtccaagatgataaaaacatttaccggcatcataaacacggagtttattttatatgtctcgcataaacattactaaaaaaatatattgttcggttttctttcacatctttaattatgaaaaagtaaatcattatgagatggacgcatcgttcgcgacagtatgtggtacatacctaacgtatttatggacgacggtaagaatgaaggtcacgtttctgtcaacaatgtcgacgcgatcgtgtaacacgactcacaatagaatctgtgaatgctctcccgatcatggatgcaaggcatgtgtttcccaaacaaaatgtggaataggatacggagtatccggagacgtcatctgttctccgtgtggtctcggaacatattctcacaccgtctcttccgcagataaatgcgaacccgtacccagaaatacctttaactatatcgatgtggaaattaacctgtatccagttaacgacacatcgtgtactcggacgaccactaccggtctcagcgaatccatctcaacgtcggaactaactattactatgaatcataaagactgcgatcccgtcttcttaataaggtagcgacttcaggtttctttacaggagaaaggtgtgcactctgaatttcgagattaaatgcaataacaaagattcttcctccaaacagttaacgaaagcaaagaatgatactatcatgccgcattcggagacagtaactctagcgtcgacatctatatactatatagtaataccaatactcaagactacgaaactgatacaatctcttatcatgtgggtaatgtagccatatgcccggtagttgcgatatacataaactgatcactaattccaaacccacccgctttttatagtaagtttttcacccataaatacaataattaatttctcgtaaaagtagaaaatatattctaatttattgcacggtaaggaagtagaatcataaagaacagtactcaatcaatagcaatcatgaaacaatatatcgtactggcatgcatgtgcctgccagtcttcagcaatcatcctcatcgtgtacggaagaagaaaacaaacatcatatgggaatcgatgttattatcaaagtcacaaagcaagaccaaacaccgaccgatgataagatttgccaatccgtaacggaaattacagagtccgagtcagatccagatcccgaggtggaatcagtcgaggatgtagatcctcctaccacttattactccatcatcggtggaggtctgagaatgaactttggattcaccaaatgtcctcagattaaatccatctcagaatccgctgatggaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgtgtcaagaatctagagtttagcgttcgtatcggagacatgtgcaaggaatcatctgaacttgaggtcaagtatgtcgacggatcggcatctgaaggtgcaaccgatgatacttcactcatcgattcaacaaaactcaaagcgtgtgtctgaatcgataactctattcatctgaaattggatgagtagggttaatcgaacgattcaggcacaccacgaattaaaaaagtgtaccggacactatattccggtttgcaaaacaaaaagttacctctcgcgacttcttctttttctgtctcaatagtgtgatacgattatgacactattcctatttcctttcagggtatcacaaaaatattaaacctctttctgatggtctcatacaaaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaatatttttattctctttctctctttgatggtctcataaaaaatatttttattctctttctctctttgatggtctcataaaa

SEQ ID NO: 3 - DNA, 3822 bp, synthetic construct
  misc_feature 1..3822: note = NCBI SARS-CoV-2 reference strain
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 4 - protein, 1273 aa, synthetic construct
  REGION 1..1273: note = NCBI SARS-CoV-2 reference strain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3822 bp, synthetic construct
  misc_feature 1..3822: note = Wuhan-Hu-1 reference strain
  source 1..3822: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 6 - protein, 1273 aa, synthetic construct; identical to SEQ ID NO: 4
  REGION 1..1273: note = SARS-CoV-2 S antigen sequence

SEQ ID NO: 7 - DNA, 1260 bp, synthetic construct
  misc_feature 1..1260: note = NCBI SARS-CoV-2 reference strain
  source 1..1260: mol_type = other DNA
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 8 - protein, 419 aa, synthetic construct
  REGION 1..419: note = NCBI SARS-CoV-2 reference strain
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 9 - DNA, 1260 bp, synthetic construct
  misc_feature 1..1260: note = Wuhan-Hu-1 reference strain
  source 1..1260: mol_type = other DNA
atgtctgataacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtagaggaacttctcctgctagaatggctggcaatggcggtgatgctgctcttgctttgctgctgcttgacagattgaaccagcttgagagcaagatgtctggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 10 - protein, 419 aa, synthetic construct; identical to SEQ ID NO: 8
  REGION 1..419: note = Wuhan-Hu-1 reference strain

SEQ ID NO: 11 - DNA, 3822 bp, synthetic construct
  misc_feature 1..3822: note = Wuhan-Hu-1 reference strain
  misc_feature 986: note = lysine
  misc_feature 987: note = valine
  source 1..3822: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 12 - protein, 1273 aa, synthetic construct
  REGION 1..1273: note = Wuhan-Hu-1 reference strain
  SITE 986: note = MISC_FEATURE - lysine
  SITE 987: note = MISC_FEATURE - valine
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3822 bp, synthetic construct
  misc_feature 1..3822: note = Wuhan-Hu-1 reference strain
  source 1..3822: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctggatcagcttcaagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 14 - protein, 1273 aa, synthetic construct
  REGION 1..1273: note = Wuhan-Hu-1 reference strain
  REGION 682..685: note = MISC_FEATURE - RRAR
  SITE 986: note = MISC_FEATURE - lysine
  SITE 987: note = MISC_FEATURE - valine
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3765 bp, synthetic construct
  misc_feature 1..3765: note = Wuhan-Hu-1 reference strain
  source 1..3765: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctggatcagcttcaagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgctaa

SEQ ID NO: 16 - protein, 1254 aa, synthetic construct
  REGION 1..1254: note = Wuhan-Hu-1 reference strain
  REGION 682..684: note = MISC_FEATURE - RRAR
  SITE 986: note = MISC_FEATURE - lysine
  SITE 987: note = MISC_FEATURE - valine
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 17 - DNA, 3822 bp, synthetic construct
  misc_feature 1..3822: note = SARS-CoV-2 S antigen
  source 1..3822: mol_type = other DNA
atgttcgtgttcctggtgctgctgccgctggtgagcagccagtgcgtgaacctgacgacccgtacacaactaccacctgcgtacacaaatagcttcaccagaggcgtgtactaccctgataaagtgttcagatcttctgtgctgcacagcacccaggatctgttcctgccgttcttctccaacgtgacctggttccatgccatccacgtgtctggaacaaacggcaccaagaggtttgacaaccctgtgctacccttcaatgacggcgtctactttgctagcaccgagaagagcaatatcatccgcggctggatcttcggcaccaccctggactccaagacccagtccctgctgattgtgaacaacgccaccaacgtggtgatcaaggtctgcgaattccagttctgtaacgacccattcctgggcgtgtattatcataagaacaataagagctggatggaatcagagttccgggtgtactcttctgccaacaactgcaccttcgagtacgtgtctcagcctttcctcatggacctggagggcaagcagggcaacttcaagaacctgagggagtttgtgttcaagaacatcgacgggtacttcaagatctacagcaagcacacacccatcaatctggtgcgagacctgcctcagggcttctcggctctggaacccttggtggatctgcccatcggcatcaacatcaccagattccagacactgctggctcttcacagaagctacctgacgcctggagatagcagcagcggctggaccgccggagccgccgcctattacgtgggctacctgcagcccagaacgttcctgcttaagtacaacgagaacggtacaattaccgatgccgtggactgcgccttagacccgctgagcgagacaaagtgtaccctgaagagctttacagtggagaaaggcatctaccagaccagtaacttcagagtgcagccaaccgaaagcatcgtgcggtttcctaatatcacaaacctgtgtccatttggcgaggtgttcaacgcgaccagattcgcgtctgtctatgcctggaacagaaagcggatcagcaactgcgtggctgactatagcgtgctttacaacagcgcctcctttagcaccttcaagtgctacggcgtcagtcccactaaactgaacgacctctgcttcaccaatgtgtacgcggatagcttcgtgatcagaggagacgaggtgcggcagattgcgcctggacagaccggcaagatcgccgactacaattacaagctgcctgacgacttcacaggctgcgtgatcgcctggaactctaacaatctggacagcaaagtcggtggaaattataactacctgtaccgcctattcagaaagtccaacctgaagccatttgaacgggacatctctaccgaaatctaccaggctggcagcactccttgtaatggcgtagagggcttcaactgttactttccactgcagagctacggcttccagcctaccaatggcgttggataccagccttacagagtggtggtgttgtcgtttgagctgcttcacgcacctgctacagtctgtggccctaagaaatctaccaatctggtaaagaacaagtgcgtgaatttcaactttaacggcctgaccggcacaggagtgctgactgagagcaataagaaattcctgcctttccagcagtttggcagagacatcgccgataccaccgacgccgtacgggatcctcagaccctggaaatcctggacatcacaccgtgtagcttcggcggagtcagcgtgatcacacctggaacaaacacatccaaccaggtggccgtgctgtaccaggacgtaaactgtacagaggtgccagtggccattcacgccgatcagctgacacctacctggcgggtttatagcacgggcagcaacgtcttccagacccgggccggctgcctgatcggcgctgaacacgtgaacaacagctacgagtgcgacatccctatcggagcgggcatctgcgccagctaccagacgcagacaaacagccctggctccgcctcttcggttgccagccagagtatcatcgcctacaccatgagtctgggagccgagaatagcgtggcctacagcaacaacagcatcgctattcccaccaatttcaccatcagcgtgaccaccgaaatcctgcctgtgagcatgaccaagaccagcgtagactgcaccatgtacatctgcggcgactcaaccgaatgtagcaacctgctgctccagtacggcagcttctgcacccagctgaacagagctctgacgggcatcgctgtggaacaagacaagaacacccaggaggtgttcgcccaagtgaaacagatctacaagactcctcctatcaaggacttcggaggcttcaatttctctcagatcttgccagatcctagcaagccaagcaagcggagcttcatcgaagaccttctgtttaacaaggtgacactggccgacgccgggttcatcaagcagtacggcgactgtctcggagacatcgctgccagagacctcatctgcgcccagaaattcaacggcctgactgtgctgcctcctctgctcacagacgagatgatcgcccagtacaccagcgccctcctggccggcaccatcaccagcggctggacgtttggtgctggtgccgccctgcagatccctttcgccatgcagatggcctatcgcttcaacggaatcggcgtgacacagaacgtgctgtacgagaaccagaagctgatcgccaaccagtttaacagcgctatcggcaagatccaagactctctgagttctacagcctctgccctgggcaaactgcaggacgtggtcaaccagaacgcccaggcgctgaataccctggtgaagcagctgagcagcaatttcggtgcgatcagcagcgtgctgaacgacatcctgagcagactggatccgcctgaggccgaagtccagatcgatagactgatcaccggcagactgcaatctctgcagacatacgtgacccagcagctgatccgggctgcggagatccgggccagcgctaacctggccgccaccaagatgagcgagtgcgtgctgggccagtctaagagggttgacttctgcggtaagggctaccacctgatgagcttcccacagtctgcgcctcacggcgttgtgttcctgcacgtgacctacgtgcctgctcaggagaagaacttcaccaccgcgccggccatctgccacgacggcaaggcccacttcccaagagagggtgtatttgtgtctaacggcacccactggttcgtgacacaaaggaacttctacgagcctcagatcataaccaccgacaacaccttcgtgagcggaaactgcgacgtggtgatcggcatcgtgaacaacaccgtgtacgaccctctgcagcctgagctggacagcttcaaagaagaactagacaagtacttcaagaaccacaccagtcccgatgtggacttgggcgatatctctggcattaacgccagcgtcgtgaacatccagaaggagatcgatagactgaacgaggtggccaagaacctgaacgaatcactgatcgacctgcaggagctgggaaagtacgagcagtacatcaagtggccctggtatatctggctgggcttcatcgctggactgatcgccatcgtgatggtgaccatcatgctgtgctgcatgacatcctgctgttcctgcctgaagggctgttgcagctgtggtagctgctgcaagttcgatgaggatgactcagagcctgtgctcaagggagtgaagctgcactacacctga

SEQ ID NO: 18 - DNA, 3822 bp, synthetic construct
  misc_feature 1..3822: note = SARS-CoV-2 S antigen
  source 1..3822: mol_type = other DNA
atgtttgtattcttagtcttgttaccactagtgtcgtctcagtgcgtcaaccttaccacgcgtactcagctaccacctgcctacaccaatagtttcacgcgaggagtttattacccggataaggtcttcagatcgtcggtcctacacagtacgcaagacttgttccttcctttcttcagtaatgtaacatggttccacgctattcacgtttccggtacaaatggaacgaaacgtttcgataacccagtcttaccgttcaatgacggtgtatatttcgcatctacagagaagtcgaatataatccgtggatggatcttcggaaccactttagattccaagactcagtcgttgttgattgtcaacaatgcaacaaatgtcgtgatcaaagtttgtgagtttcaattctgtaatgacccattcttaggtgtgtactaccataagaataacaagtcatggatggaatcagaatttcgtgtctattcttcggcgaacaactgcactttcgagtatgtgagtcaacctttcctaatggacctagagggaaagcagggtaactttaagaatcttcgagaattcgtctttaagaatattgacggttacttcaagatctattccaagcacactcccataaacttggtccgagatctaccacagggtttctccgctctagaacccttagtggatcttcctataggaatcaacatcacgagattccagacattgttagcattgcatcgatcctatcttacgcctggagattcttcatcgggatggactgcaggagccgctgcgtactatgtgggatacttacagcctcgaacattcctacttaagtacaatgagaacggaactatcacagacgcggtggactgtgctttggacccattgtcggagacaaagtgtacccttaagtcattcactgtcgagaagggaatttatcaaacgtccaactttcgtgtgcagccaacagaatccatagtcagatttccaaacattacgaatctttgcccgttcggtgaagtctttaacgccacccgtttcgcatccgtttacgcctggaacagaaagagaatctcgaactgcgtggcggactattctgtactttataactcagcttcattctcaacattcaagtgttatggtgtgtcgccaacaaagctaaatgatctatgcttcaccaacgtatacgcggattcgttcgtaattagaggtgatgaggtgcgacagatcgctcctggacagacaggaaagatagctgattacaattacaagttaccggatgatttcaccggttgcgtgatcgcttggaattctaacaatttggactccaaagttggaggaaactacaactacttatatcgattatttagaaagtccaacttgaaacctttcgagcgtgacataagtacggagatctatcaggccggatcgacaccatgcaatggtgtggagggtttcaattgttactttccacttcaaagttacggattccaaccgacgaacggagtgggttaccagccgtatcgagtcgtagtcctatcatttgagttgctacatgctccagcgaccgtttgcggacctaagaagtccacgaacctagtaaagaacaaatgcgtcaatttcaacttcaacggtttgactggaactggagtgttaactgagtccaacaagaaatttctaccgttccaacaatttggtagagacattgcagacacgacggatgcggtacgtgaccctcaaactttagagatcttagacataacgccttgctccttcggaggagtttccgtgattacgcctggaaccaatacctcaaaccaagtcgctgtgctataccaggatgtgaactgcaccgaggttccagtagctattcacgctgatcagctaacgcctacttggagagtatattcgactggatctaacgtgttccagacacgagctggatgtcttattggtgcagagcatgtcaataactcttatgaatgtgatattccaattggtgcgggaatttgcgcatcataccagacacaaaccaacagtcccggatcagcttcatccgtcgcttcgcaatctatcattgcttacactatgagtctaggagctgagaactcagttgcgtacagtaacaacagtatcgctatacctactaactttacaataagtgtcaccactgaaatcttaccagtatccatgacgaagacctccgttgattgtacaatgtatatatgtggagattcaaccgagtgcagtaacttgttacttcagtatggatccttctgcacacaattgaatcgagcccttaccggtatcgccgtagaacaagataagaacacacaggaggtctttgctcaggtcaagcagatttacaagacaccgccaatcaaggacttcggtggattcaatttctctcagatccttcccgacccgtccaaaccgtctaaacgatcgtttatcgaagatttattgttcaataaggttactttggcggacgctggtttcattaagcagtatggagactgtcttggtgacattgctgcgcgtgaccttatatgcgcccagaagttcaatggacttacagttctaccgccacttctaaccgatgaaatgatcgcgcagtatacatctgcattgttggccggtacgattacatctggatggactttcggtgctggagcagctttgcaaattccgttcgcgatgcaaatggcatacagattcaacggaatcggagtaacgcagaatgtgctatatgagaaccagaaacttatagcaaaccaattcaactcggccatcggtaagattcaggattcgttgtcgagtacagccagtgcgcttggaaagctacaggacgtagttaatcagaatgcgcaagccttgaacacgttagttaagcaactatcttccaactttggtgccatttcttctgtgttgaacgacattctatctcgattagacccacctgaggccgaggttcaaattgatcgattaataaccggaagattgcagtcgctacaaacatatgtaactcagcaattaattagagctgcggaaattcgagcttctgcgaaccttgccgcgacgaagatgtcggaatgcgtgttaggtcagtcaaagcgagtggacttctgtggaaagggataccaccttatgtcctttccgcaatctgcgccacacggtgtggtgtttctacatgtgacctatgtacctgcacaagagaagaactttacgacagcaccagccatctgtcatgatggaaaggcccactttccacgagaaggagtatttgtgtccaatggtacacactggtttgttacccaacgaaatttctatgaaccgcagatcatcacaactgataacacgttcgtctctggaaactgtgacgtagttataggaattgtcaacaatactgtctacgacccattacagccggagcttgattcattcaaagaggagttggataagtacttcaagaaccacaccagtccggatgtggatttaggtgatatctccggtattaacgcttccgtcgttaatatacagaaggaaatcgaccgacttaatgaagtagcaaagaaccttaatgaatccctaatcgatctacaagaacttggtaaatatgagcaatatatcaagtggccttggtacatttggttgggtttcattgcaggattgattgcgattgttatggtcacaataatgctttgttgtatgacgagttgttgttcgtgtttgaagggatgctgctcatgtggttcttgttgtaaattcgatgaagacgattcagaaccggtattgaaaggagttaagttgcactacacgtaa

SEQ ID NO: 19 - DNA, 1260 bp, synthetic construct
  misc_feature 1..1260: note = SARS-CoV-2 N antigen
  source 1..1260: mol_type = other DNA
atgtctgataatggcccgcagaatcagcgcaacgcacctagaatcacatttggcggtcccagcgactctaccggcagcaaccagaacggagaacggagcggagccagaagcaagcaaagacgccctcagggactgcctaacaacaccgccagctggttcactgctctgacccagcacggtaaagaggatctgaagtttcctagaggccaaggagttcctatcaacaccaacagcagcccagacgatcagatcggctactatagaagagccacaagaagaatccgaggcggcgacggcaagatgaaggacctgtcacctagatggtacttctactacctgggcacaggtcccgaagccgggctgccttatggcgcaaataaggacggaatcatctgggtggccacagaaggcgccctgaatacacccaaggaccacatcggcaccagaaacccggccaacaacgccgctatagtgctgcagctgccacagggtaccacgctgccaaagggcttctacgctgagggaagcagaggaggctctcaggcctcttccagaagctcttccagaagcagaaactccagcagaaatagcactcccggcagtagcagaggcacaagccctgctagaatggccggtaatggaggcgacgccgcgctggccctcctgctgctggatcggctgaaccagctggaatctaagatgagcggcaagggccagcaacagcagggccagacagtgaccaagaaatctgccgccgaggctagcaagaaacctcggcagaagaggaccgccaccaaggcctacaacgtgacacaggccttcggtcgtcgcggtcccgagcagacacagggcaacttcggcgaccaggagctgatcagacaaggcacagattacaaacattggcctcagatcgcccagttcgctccaagcgcctctgctttcttcggcatgagcagaatcggcatggaagtgacgccttcaggcacatggctgacctacaccggcgccatcaagctggacgataaggacccgaacttcaaggaccaggtgatcctgctgaacaagcacatcgacgcctacaagaccttccctcctacagagcctaagaaggacaagaagaagaaagccgatgaaacccaggccctgcctcagcggcagaagaagcaacagaccgtgaccctgctgcctgccgccgatctggacgacttcagcaaacaactgcagcaatccatgagctctgccgatagcacccaggcctga

SEQ ID NO: 20 - DNA, 1260 bp, synthetic construct
  misc_feature 1..1260: note = SARS-CoV-2 N antigen
  source 1..1260: mol_type = other DNA
atgtcggataacggtccacagaaccaaagaaatgcacctcgaattacgtttggtggtccttccgactccactggatcgaatcagaatggtgagagaagtggtgcgagatctaaacaacgaagaccgcaaggtttgccgaacaatacagcctcgtggttcaccgcccttacacaacatggtaaagaggacctaaagtttcctcgtggacaaggtgtgcctatcaatacgaattcgagtccggatgatcagattggttattatcgacgagccacgagacgaattcgaggaggtgacggaaagatgaaggacttgtcaccacgttggtatttctactatttgggtacgggacctgaggccggacttccatatggagctaacaaggacggtatcatttgggtggcgaccgaaggagctcttaacacaccaaaggatcatataggtacgcgtaaccctgctaacaacgcagccattgtgttacagttaccacaaggtacgacacttcccaaaggattctatgcggaaggttcccgtggaggatcgcaggcctcgtcacgttcgtcctccagatcgagaaactcttcacgtaactctacgcctggtagttcccgaggaacgagtccggcaagaatggcaggtaatggaggtgatgctgccttagccctattactattagacagacttaatcaattagagagtaagatgtcaggaaagggtcagcaacaacaaggtcagacagtgacaaagaagtctgcggctgaggcatctaagaaaccgagacagaagagaacagctactaaggcgtacaacgtgactcaagcatttggtcgtcgtggaccggaacagactcagggtaactttggtgaccaagagttaatacgtcaaggaactgactacaagcactggcctcagatcgcacaattcgcaccgtcggcctcggctttcttcggaatgtcacgaataggtatggaggtaacaccctcgggaacgtggctaacgtacactggtgcgattaagcttgatgacaaggaccctaacttcaaagaccaagtcatcctacttaacaagcacatagacgcgtataagaccttccctccaactgaacccaagaaagataagaagaagaaagcagatgaaactcaagcattgcctcagcgtcagaagaagcaacagacggttacacttctacctgctgctgatcttgacgatttctcaaagcaattacaacagtcaatgagttcggctgactcaacccaggcttaa

SEQ ID NO: 21 - DNA, 2097 bp, synthetic construct
  misc_feature 1..2097: note = Wuhan reference strain
  source 1..2097: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcataa

SEQ ID NO: 22 - protein, 698 aa, synthetic construct
  REGION 1..698: note = Wuhan reference strain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMS

SEQ ID NO: 23 - DNA, 2043 bp, synthetic construct
  misc_feature 1..2043: note = Wuhan reference strain
  source 1..2043: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattcttaa

SEQ ID NO: 24 - protein, 680 aa, synthetic construct
  REGION 1..680: note = Wuhan reference strain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNS

SEQ ID NO: 25 - DNA, 621 bp, synthetic construct
  misc_feature 1..621: note = Wuhan reference strain
  source 1..621: mol_type = other DNA
atgtttgtctttcttgtcttattgccactagtctctagtaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtt

SEQ ID NO: 26 - protein, 207 aa, synthetic construct
  REGION 1..207: note = Wuhan reference strain
MFVFLVLLPLVSSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 27 - DNA, 708 bp, synthetic construct
  misc_feature 1..708: note = Wuhan reference strain
  source 1..708: mol_type = other DNA
atgtttgtctttcttgtcttattgccactagtctctagtagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttc

SEQ ID NO: 28 - protein, 235 aa, synthetic construct
  REGION 1..235: note = Wuhan reference strain
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTIYQAGSTPCNGVEGFNCYFPLQSYGFQPTNGVGYQPYRVVVLSFELLHAPATVCGPKKSTNLVKNKCVNF

SEQ ID NO: 29 - DNA, 3813 bp, synthetic construct
  misc_feature 1..3813: note = Wuhan-Hu-1 reference strain
  source 1..3813: mol_type = other DNA
atgtttgtctttcttgtcttattgccactagtctctagtcagtgtgttaactttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgctaaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtggtctccctcagggtttctcggctttagaaccattggtagatttgccaataggtattaacatcactaggtttcaaactttacatataagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttaaaggatttaattgttactttcctttacaatcatatggtttccaacccacttatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgtagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 30 - protein, 1270 aa, synthetic construct
  REGION 1..1270: note = Wuhan-Hu-1 reference strain
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - DNA, 3813 bp, synthetic construct
  misc_feature 1..3813: note = Wuhan-Hu-1 reference strain
  source 1..3813: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatatctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttaccacaagaacaacaagagttggatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttgaaggatttaattgttactttcctttacaatcatatggtttccaacccacttatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgatgacactactgatgctgtccgtgatccacagacacttgagattcttgacattacaccatgttcatttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcatcggcgggcacgtagtgtagctagtcaatccatcattgcctacactatgtcacttggtgcagagaattcagttgcttactctaataactctattgccatacccataaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatccttgcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacacacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 32 - protein, 1270 aa, synthetic construct
  REGION 1..1270: note = Wuhan-Hu-1 reference strain
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 33 - DNA, 3822 bp, synthetic construct
  misc_feature 1..3822: note = Wuhan-Hu-1 reference strain
  source 1..3822: mol_type = other DNA
atgtttgtctttcttgtcttattgccactagtctctattcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttgggtgtttattaccacaagaacaacaagagttgcatggaaagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattaccggtatagattgtttaggaagtctaatctcaaacctttcgagagagatatttcaactgaaatctatcaggccggtagcacaccttgtaatggtgttgaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 34 - protein, 1273 aa, synthetic construct
  REGION 1..1273: note = Wuhan-Hu-1 reference strain
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 35 - DNA, 3822 bp, synthetic construct
  misc_feature 1..3822: note = Wuhan-Hu-1 reference strain
  source 1..3822: mol_type = other DNA
atgtttgtctttcttgtcttattgccactagtctctagtcagtgtgttaactttacaaacagaactcaattaccatctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaattatccattcttgggtgtttattaccacaagaacaacaagagttggatggaaagtgagttcagagtttattctagtgcgaataattgcactttcgaatatgtctctcagcctttccttatggaccttgaaggtaaacagggtaatttcaagaatcttagcgaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaacgattgctgattataattataaattaccagatgactttacaggctgcgttatagcttggaattctaacaatcttgattctaaggttggtggtaattataattacctgtatagattgtttaggaagtctaatctcaaacctttcgagagagatatttcaactgaaatctatcaggccggtagcacaccttgtaatggtgttaaaggatttaattgttactttcctttacaatcatatggtttccaacccacttatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtatttcaaacacgtgcaggctgtttaataggagctgaatatgtcaacaactcatatgagtgtgacatacccattggtgcaggtatatgcgctagttatcagactcagactaattctcctcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctattaagatgtcagagtgtgtacttggacaatctaagagagttgatttctgtggaaagggctatcatcttatgtccttccctcagtcagcacctcatggtgtagtcttcttgcatgtgacttatgtccctgcacaagagaagaacttcacaactgctcctgccatttgtcatgatggtaaagcacactttcctcgtgaaggtgtctttgtttcaaatggcacacactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcatttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 36 - protein, 1273 aa, synthetic construct
  REGION 1..1273: note = Wuhan-Hu-1 reference strain
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 37 - DNA, 3822 bp, synthetic construct
  misc_feature 1..3822: note = Wuhan-Hu-1 reference strain
  source 1..3822: mol_type = other DNA
atgtttgtcttcttggtccttctgccgcttgtttcttcacaatgtgtgaatttgaccacccgcacccagctgcctcctgcatatacgaattcattcactcgcggcgtgtactatccagataaagtctttcgatccagtgtactgcattcaacgcaagatctctttcttccattcttcagcaatgtgacctggttccacgcgatacacgtaagcggaaccaacggaacaaagagatttgacaatccagtactcccgtttaatgacggtgtctactttgcctctaccgagaagtctaatatcataagagggtggatcttcggtacgacgctcgattctaagacgcaaagcctgcttattgttaataatgccaccaatgtcgtgattaaggtatgcgaatttcaattctgtaacgatccattccttggagtttactatcataagaacaacaaatcctggatggaatcagagtttcgggtctacagctccgcaaataactgtacgtttgagtacgtgtcccagccctttcttatggatctcgaaggtaaacaaggcaatttcaagaatcttagagaattcgtcttcaagaacatcgatggatactttaagatatactcaaagcatacgccgataaacctcgtgcgcgacctgccacagggattcagtgcgcttgagccattggtagatcttcctataggaatcaacataactagatttcagacactgctggcgcttcatagatcctatctgactccaggtgactcctccagtggatggactgccggcgccgcggcttactacgtgggttatcttcagcctcgaacgtttcttctgaagtataacgagaacgggacgataaccgatgcggtggattgcgcactggaccctcttagcgaaactaaatgtacccttaagagctttacggttgagaagggtatttatcagactagtaactttcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcaacttcaatggactcacagggacgggcgtgcttacggagtcaaacaagaagtttcttcctttccagcaattcggcagggacattgccgatacaacggacgcagttagggaccctcaaactcttgaaatcttggacataactccttgcagctttggcggcgtgagtgtgataacacctggtaccaatacgtccaatcaggtagcagtactgtatcaggatgtcaattgcaccgaagtccctgtcgccattcatgccgaccaactgacgccaacatggcgagtatacagtaccgggtcaaacgtgttccagaccagggcaggctgtctcattggtgcggaacacgttaacaacagttacgaatgcgatatacctattggtgctggcatctgtgcctcttaccagacccaaactaactcaccacgaagggcaagatcagttgccagtcagtctattatcgcttacacgatgtcactgggagctgagaactctgttgcttactcaaataattcaattgccatcccaacgaatttcactattagtgtaacaacggaaattctgcccgtgtccatgaccaagacgtccgtcgattgtaccatgtacatatgcggtgacagcaccgaatgtagcaacctcctgctccaatatggtagcttctgcacacagctcaacagagcacttacaggtatcgccgtagagcaagacaagaacacacaagaagtgttcgctcaggtgaagcagatctacaagactccaccgataaaggacttcggtggctttaactttagccaaatcctgcccgacccttctaagccttctaagagatcctttattgaggatctcttgttcaacaaagtcaccctcgctgacgcagggtttataaagcagtatggtgactgtctcggcgatatagcagccagagacttgatctgcgcccagaagttcaacgggcttacggttcttccgccgctgcttactgatgaaatgatagctcaatacacatctgctcttctcgctgggacaataacttccgggtggacgttcggagcgggtgccgccttgcaaattccctttgcgatgcagatggcttatcgcttcaatggcataggcgttacccagaacgtgctgtacgagaatcagaaattgatcgccaaccagtttaattctgccatcggaaagatccaagattccctgtctagcactgcgtcagccctcggtaaactgcaggatgtggttaatcagaatgctcaggctctcaacactctggttaaacagctgtctagcaacttcggtgccatctcttcagttcttaacgatatattgagtaggcttgataaagtcgaggcagaggttcaaattgaccggttgatcacgggcaggcttcaatcactgcaaacctacgtcactcaacaactgattcgcgcagcggaaatacgagcttcagctaatctcgcagcgactaagatgtccgagtgtgtgctcggtcagagcaagagagttgacttctgtgggaaagggtaccatctcatgtcatttcctcaatctgcaccgcacggagtagtattcctccacgttacttatgtgccggcacaagagaagaacttcactactgcgccagccatttgtcacgacgggaaagcgcactttcccagggaaggagtcttcgtatcaaacggtacgcattggttcgtaacgcaaagaaacttctatgaaccacaaattatcacgacggataatacgtttgtctccgggaactgtgacgtggtaattggtattgtaaataacacagtttatgacccattgcaacctgaactggacagcttcaaggaggaattggacaagtatttcaagaaccataccagtccggacgtagacctcggcgatatctcaggaattaacgctagcgtggtgaatattcagaaagaaatagatcgcctgaacgaagtagcgaagaaccttaacgaaagtttgattgatcttcaggagctgggtaaatacgagcaatacataaagtggccgtggtacatctggctcggctttatagctggcctgatcgccattgtgatggtgactattatgttgtgttgcatgactagctgctgttcatgtcttaagggatgctgcagctgcggtagctgctgtaaatttgacgaagacgatagcgagccggtactgaaaggtgtgaaacttcattacacataa

SEQ ID NO: 38 - DNA, 3813 bp, synthetic construct
  misc_feature 1..3813: note = B.1.351 variant
  source 1..3813: mol_type = other DNA
atgttcgtgtttcttgtgttgttgccactggtttcatcccaatgtgtgaactttaccaccaggacgcagttgccaccagcttatactaattccttcactagaggagtgtactacccggacaaggtattccggtctagtgtccttcactccacgcaagacctgttccttccgttcttctccaatgtaacctggtttcacgccattcatgtttcaggcacgaatggcactaagcgcttcgccaacccagtactgccgtttaatgatggagtctacttcgcttcaacagagaagagcaatatcatacgaggctggatctttggtactaccttggattccaagacgcaatcccttcttattgtcaataatgcgaccaacgttgtaatcaaagtatgcgagtttcaattctgcaacgacccattcttgggcgtctattatcataagaataataagtcttggatggagtctgagttcagagtgtattcatcagcgaacaattgcacattcgaatatgtatctcagccctttctcatggatctcgaagggaagcaagggaacttcaagaatctccgcgagttcgtctttaagaacattgacggctatttcaagatatacagcaaacatactccaatcaacctggttcgaggactccctcagggcttctccgcgctggaacccttggtggacctcccaataggcataaatatcacgaggttccaaacattgcacataagctatctgactccgggagacagttccagcggttggactgcaggcgcggctgcgtactacgtgggctacttgcaacctaggacgtttctcttgaaatacaatgagaacggcaccatcacggatgcagttgactgtgctctcgacccactctccgaaaccaagtgtacgctcaagtcatttaccgtcgagaaaggaatctaccaaactagtaatttccgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcaacttcaacggtctcacagggaccggtgtattgacagagtcaaacaagaagttcttgccctttcaacagttcggcagggatattgctgacactaccgatgcggttagggaccctcaaaccctggagattcttgacattacaccgtgcagcttcggtggagtcagcgttatcacaccaggcactaatacgtccaatcaagttgctgtactgtaccaaggagtcaattgtaccgaggtaccagtagcgattcacgccgaccaactcacgcccacatggcgcgtctatagcacaggcagcaatgtatttcaaacgcgcgcaggatgtctcattggagcggagcatgtcaataactcatacgaatgtgacattccaataggagcgggtatttgtgccagctaccagacgcaaacgaattctccacgccgagccagatcagtcgcgagtcagagtataatagcgtacacgatgtctctgggcgtcgagaattccgtcgcatactcaaacaattccattgcgatcccaacgaacttcacaatatccgtcaccactgaaatcctccctgttagcatgactaagacaagcgtggactgcactatgtatatttgtggtgattccacggaatgctctaatctgcttctccaatatggaagtttctgcacccaactgaacagggcgcttacaggcatagcagttgaacaagacaagaatacccaagaggtctttgcccaggtgaaacaaatttataagactcctccgatcaaggactttggagggttcaacttctcccagatacttcccgacccgagcaagccctccaagagatcgtttatagaagatctcctctttaacaaggtcacgttggctgatgccggctttattaaacaatacggtgactgtctcggagatatcgcggctagagacctgatatgtgcacagaaatttaacgggctcaccgtattgccaccgctgctgacggatgaaatgatcgcccaatacacctccgcgttgttggccgggaccattacttctggttggacatttggtgcaggagccgcacttcagataccatttgctatgcaaatggcgtatcgattcaacgggattggtgttacgcagaatgtcctctatgagaaccagaaactgatagcgaatcagttcaacagcgctattggcaagatccaagattctttgtcatcaaccgcgagtgccctgggtaaactgcaagatgtagtcaaccagaacgcacaagcccttaatacccttgtcaagcagctctcatccaactttggtgctatttcttctgttctgaatgatattctcagtcgactggacaaggtagaagcagaggttcagatagatcgcctcattactgggcgccttcagtcacttcagacttatgttacccaacaactcattagggcagccgaaatccgagcttcagcgaatctcgccgctactaagatgagcgagtgcgtacttggtcaatccaagagagtagatttctgtgggaagggatatcacctgatgtctttcccacagagcgcgccacacggtgtagtattcttgcatgttacatacgttccagcacaagagaagaactttactacagcaccggcaatctgccatgatgggaaggcacactttccgagggagggagtctttgtatccaatggcacccactggtttgttactcaaagaaatttctatgagccacagatcattactacggacaacacctttgtaagcgggaactgtgacgtagtcataggcatagtaaataataccgtttacgatccactgcagccggagctcgattcattcaaagaggaactggacaaatacttcaagaaccacacgtcacctgacgttgaccttggtgatatttctggcataaacgcctctgttgtgaatatccagaaggagatagacagactgaacgaggtggcaaagaatctgaacgaatcattgatcgatttgcaagagttgggaaagtacgaacagtatataaagtggccgtggtatatatggctagggttcattgctggacttatagcaattgtgatggtaacgatcatgttgtgttgcatgacttcctgctgcagttgcctcaaaggatgttgttcctgtggatcatgctgtaagtttgatgaagatgactccgaacccgtgcttaaaggagtgaagttgcactatacttaa

SEQ ID NO: 39 - DNA, 3813 bp, synthetic construct
  misc_feature 1..3813: note = B.1.1.7 variant
  source 1..3813: mol_type = other DNA
atgtttgtctttctcgtacttctgcctctggtcagcagccaatgtgtgaacctcacaaccaggactcaattgccgccagcgtatacgaacagcttcacgcgcggcgtttattatccggataaggtattccgcagcagtgtgcttcatagtacacaggacctattcttgccgttcttctcaaacgtgacatggttccatgccattagtggcacaaacgggacaaagcggttcgacaacccggttcttccctttaatgacggagtatactttgcaagcacggagaagagcaacatcattagagggtggatctttgggaccacattggattccaagacgcagtccctgctgatagtcaacaacgctaccaacgtcgtcatcaaggtttgtgaattccaattctgcaacgatccgtttctgggagtttatcacaagaacaataaatcttggatggagagtgaatttcgcgtttactcatccgcaaataattgtaccttcgagtacgtgtcccagcctttcctcatggacttggagggtaagcagggaaactttaagaatctgagagaatttgtctttaagaacattgacgggtacttcaagatatactcaaagcatacacccattaacctggtgagggatttgccgcagggattctcagcactggagccactcgttgatctgccgatagggataaatatcacgcggttccaaactcttcttgctttgcaccgctcttaccttactccaggagactcttcctctgggtggacggctggagcggctgcttattacgtcgggtacttgcagccgcggaccttccttctgaaatacaatgagaacggtacaattactgacgctgtggattgcgccctggatccgctgtctgagacgaaatgcacgcttaagtcatttacggtcgagaagggaatctatcagacatccaactttcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactacctgtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttcaactttaatgggctgaccggaaccggagtcctcacagagagcaacaagaaattccttccgtttcagcagttcggtagggatatagatgacacgactgatgcggtccgggatcctcaaacgcttgagattcttgacatcacgccgtgcagctttggtggagttagtgtaattactccgggtaccaatactagcaaccaagttgccgtactttatcaaggtgtaaactgcactgaagtaccggtcgccattcatgcagaccagctcacaccaacgtggagggtttattccactggatccaatgtctttcaaacaagagcgggctgtttgattggagcggaacatgttaacaacagttacgagtgtgacatacctataggagctggcatctgtgcgagctaccagacacaaaccaactctcatagacgggcacgctccgtggcatctcagtctatcatagcgtatacgatgagtttgggagcagagaattctgttgcatactctaacaatagtatcgccattccgattaactttaccatctcagtaacgaccgaaatacttcccgtctctatgactaagacaagtgtggactgcaccatgtacatttgcggtgactctaccgagtgctctaatttgctattgcaatatgggtcattctgtactcagctgaatagggccttgactgggatagccgtcgaacaagacaagaatacgcaggaggtattcgcccaagtaaagcaaatatataagacacctcccatcaaagactttggagggttcaatttcagtcagatcctacccgatccaagtaagccatcaaagaggagcttcatcgaggatctgctattcaataaggttaccctcgctgacgctggcttcatcaagcagtatggagattgcctcggcgacatagcagcacgagatcttatttgtgcacagaaatttaatggactcaccgttctcccacctctcctcacagacgaaatgatcgcacagtacacttctgccctcctcgccgggacgattactagcggctggacatttggcgctggtgctgctctgcagattccatttgctatgcagatggcttatcgctttaacgggatcggagtcacgcagaatgttctttacgagaaccagaaacttattgcgaaccagttcaactctgcgattgggaagatacaggatagcctttcctctactgcctctgcacttggtaagttgcaagacgttgtgaaccagaatgctcaagcattgaacacactggtgaagcagcttagttctaacttcggtgctatcagtagtgttctcaatgatatcctggctcggctcgacaaggtggaggccgaagtacaaatcgatagactcataaccggaagactgcaatccttgcagacatacgttacccagcaacttatacgggccgccgaaatacgagcgtcagcaaacttggccgccaccaagatgtctgaatgcgtcttgggccaaagcaagcgagtggacttctgcggtaaaggttatcatttgatgtcattccctcaatccgctcctcatggagtggtgtttcttcatgttacttacgtgcctgcacaagagaagaatttcaccactgctccagccatttgtcatgatgggaaagcccatttcccaagggaaggagtattcgtctcaaatggaacacattggttcgtaacacagagaaacttctacgaaccacaaatcataacaacccacaatacgttcgtttcagggaactgcgatgtcgtaatcggaatagtaaacaatacggtctatgacccattgcaaccagagcttgactctttcaaagaagagttggataagtactttaagaatcatacgtctccggacgtagatcttggcgatataagtggcattaatgcttctgttgtgaatatacagaaagaaatcgacagacttaacgaggtcgcgaagaatctgaacgagtcattgatagaccttcaggaattgggcaagtatgaacagtacattaaatggccgtggtacatatggctagggtttatagcaggccttatagcaatcgtcatggttaccatcatgctttgctgtatgaccagttgctgctcatgcctgaagggttgttgttcctgcggatcttgctgtaagttcgatgaagacgattctgagcctgtcctcaaaggagtaaagctccattacacctaa

SEQ ID NO: 40 - DNA, 2094 bp, synthetic construct
  misc_feature 1..2094: note = open reading frame
  source 1..2094: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcacgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagtcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttctaa

SEQ ID NO: 41 - protein, 16 aa, synthetic construct
  REGION 1..16: note = signal peptide of S antigen
MFVFLVLLPLVSSQCV

SEQ ID NO: 42 - protein, 6 aa, synthetic construct
  REGION 1..6: note = GS linker
GSGSGS

SEQ ID NO: 43 - protein, 697 aa, synthetic construct
  REGION 1..697: note = encoded protein
  REGION 1..16: note = MISC_FEATURE - signal peptide of S antigen
  REGION 240..245: note = MISC_FEATURE - GS linker
  REGION 469..474: note = MISC_FEATURE - GS linker
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 44 - DNA, 2193 bp, synthetic construct
  misc_feature 1..2193: note = open reading frame
  source 1..2193: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcacgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagtcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttcggatcaggaagtggatcaggttatatccctgaagcgccaagagacggacaggcttatgtcagaaaggatggtgaatgggtcttgctttcgacgttcctataa

SEQ ID NO: 45 - protein, 27 aa, synthetic construct
  REGION 1..27: note = T4 Foldon domain
GYIPEAPRDGQAYVRKDGEWVLLSTFL

SEQ ID NO: 46 - protein, 730 aa, synthetic construct
  REGION 1..730: note = encoded protein
  REGION 1..16: note = MISC_FEATURE - signal peptide of S antigen
  REGION 240..245: note = MISC_FEATURE - GS linker
  REGION 469..474: note = MISC_FEATURE - GS linker
  REGION 698..703: note = MISC_FEATURE - GS linker
  REGION 704..730: note = MISC_FEATURE - T4 Foldon domain
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSGYIPEAPRDGQAYVRKDGEWVLLSTFL

SEQ ID NO: 47 - DNA, 2307 bp, synthetic construct
  misc_feature 1..2307: note = ORF of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  source 1..2307: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcaggagctaccaatttcagtctactaaagcaagcgggtgatgttgaggagaatcccggacctatgttcgtgtttcttgtgttgttgccactggtttcatcccaatgtgtgcgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagtggagaaggtcgtggttctctacttacgtgcggagatgttgaggagaatccaggacctatgtttgtctttctcgtacttctgcctctggtcagcagccaatgtgtgcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttctaa

SEQ ID NO: 48 - protein, 768 aa, synthetic construct
  REGION 1..768: note = encoded protein sequence of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  REGION 1..16: note = MISC_FEATURE - signal peptide of S antigen
  REGION 240..245: note = MISC_FEATURE - GS linker
  REGION 266..281: note = MISC_FEATURE - signal peptide to S antigen
  REGION 505..510: note = MISC_FEATURE - GS linker
  REGION 529..545: note = MISC_FEATURE - signal peptide of S antigen
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSGATNFSLLKQAGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSGEGRGSLLTCGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 49 - DNA, 2604 bp, synthetic construct
  misc_feature 1..2604: note = ORF of the RBD domain of the Wuhan-Hu-1 reference strain, the RBD domain of the B.1.351 variant, and a RBD domain combining N501Y and L452R mutations of the B.1.1.7 and B.1.429+B.1.427 variants
  source 1..2604: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcaggttacattccagaagcaccacgagacggtcaggcatacgtgcgtaaggacggtgagtgggtgctactaagtacattcttaggttcgggttcgggttctggagctaccaatttcagtctactaaagcaagcgggtgatgttgaggagaatcccggacctatgttcgtgtttcttgtgttgttgccactggtttcatcccaatgtgtgcgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagtggatatataccggaggctcctcgtgatggacaagcctatgtacgtaaagatggagaatgggtattattgagtacgttcttaggatctggatccggatcaggagaaggtcgtggttctctacttacgtgcggagatgttgaggagaatccaggacctatgtttgtctttctcgtacttctgcctctggtcagcagccaatgtgtgcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttcggatcaggaagtggatcaggttatatccctgaagcgccaagagacggacaggcttatgtcagaaaggatggtgaatgggtcttgctttcgacgttcctataa

SEQ ID NO: 50 - protein, 867 aa, synthetic construct
  REGION 1..867: note = encoded protein sequence of the RBD domain of the Wuhan-Hu-1 reference strain, the RBD domain of the B.1.351 variant, and a RBD domain combining N501Y and L452R mutations of the B.1.1.7 and B.1.429+B.1.427 variants
  REGION 1..16: note = MISC_FEATURE - signal peptide of S antigen
  REGION 240..245: note = MISC_FEATURE - GS linker
  REGION 246..272: note = MISC_FEATURE - T4 Foldon domain
  REGION 273..278: note = MISC_FEATURE - GS linker
  REGION 299..314: note = MISC_FEATURE - signal peptide of S antigen
  REGION 538..543: note = MISC_FEATURE - GS linker
  REGION 544..570: note = MISC_FEATURE - T4 Foldon domain
  REGION 571..576: note = MISC_FEATURE - GS linker
  REGION 596..611: note = MISC_FEATURE - signal peptide of S antigen
  REGION 835..840: note = MISC_FEATURE - GS linker
  REGION 841..867: note = MISC_FEATURE - T4 Foldon domain
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSGYIPEAPRDGQAYVRKDGEWVLLSTFLGSGSGSGATNFSLLKQAGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSGYIPEAPRDGQAYVRKDGEWVLLSTFLGSGSGSGEGRGSLLTCGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSGYIPEAPRDGQAYVRKDGEWVLLSTFL

SEQ ID NO: 51 - DNA, 2235 bp, synthetic construct
  misc_feature 1..2235: note = ORF of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  source 1..2235: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcacgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagtcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttcggatcaggaagtggatcatggtacatatggctagggtttatagcaggccttatagcaatcgtcatggttaccatcatgctttgctgtatgaccagttgctgctcatgcctgaagggttgttgttcctgcggatcttgctgttaa

SEQ ID NO: 52 - protein, 744 aa, synthetic construct
  REGION 1..744: note = encoded protein sequence of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  REGION 1..16: note = MISC_FEATURE - signal peptide of S antigen
  REGION 240..245: note = MISC_FEATURE - GS linker
  REGION 469..474: note = MISC_FEATURE - GS linker
  REGION 698..703: note = MISC_FEATURE - GS linker
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSWYIWLGFIAGLIAIVMVTIMLCCMTSCCSCLKGCCSCGSCC

SEQ ID NO: 53 - DNA, 2730 bp, synthetic construct
  misc_feature 1..2730: note = ORF of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  source 1..2730: mol_type = other DNA
atgtttgtcttcttggtccttctgccgcttgtttcttcacaatgtgtgcgcgtgcagcccacggaatcaattgtgcggtttcccaatattacaaacttgtgccctttcggcgaggtcttcaatgcaacgcgctttgcgtcagtttacgcctggaataggaagaggatatcaaactgcgtggctgactattctgtgctttacaactctgctagtttcagtacctttaagtgttacggtgtgagtccgacgaagctcaatgatctttgtttcactaatgtatacgcggacagtttcgttatacgaggagacgaggtccggcaaatcgcacccggccaaacgggaaagattgctgactacaactacaaattgcccgatgactttacgggatgcgtcattgcatggaattctaataatctggatagcaaagtaggtggaaactataattatctctaccggctcttccgaaagagtaatctcaaaccgtttgaaagagatattagcacggaaatatatcaagccggctccacaccatgtaatggagttgaaggctttaattgttatttcccgttgcaaagttacggatttcagcccacgaacggtgtgggttatcaaccgtatcgggtcgtagttctcagcttcgagctgcttcatgcgcccgcgactgtttgcggtcctaagaagtcaaccaatctcgtaaagaacaagtgtgtgaacttcggatctggatcaggttcatggtacatctggctcggctttatagctggcctgatcgccattgtgatggtgactattatgttgtgttgcatgactagctgctgttcatgtcttaagggatgctgcagctgcggtagctgctgtggttcgggttcgggttctggagctaccaatttcagtctactaaagcaagcgggtgatgttgaggagaatcccggacctatgttcgtgtttcttgtgttgttgccactggtttcatcccaatgtgtgcgcgttcagcctacagagtcaattgtgcgatttcctaacatcactaacctgtgcccgtttggagaagtatttaatgcgactcgcttcgctagtgtctatgcgtggaatcgaaagcggatctcaaactgtgtggcagattattccgtcctgtataatagtgccagcttctcaacgtttaaatgctacggagttagcccaacgaagctgaacgacttgtgctttacgaacgtgtatgccgattccttcgtcatccgcggagacgaagttcgccaaatcgcaccaggtcaaacgggtaatatagcggactacaactacaagctgcccgacgatttcactgggtgcgtgatcgcgtggaatagtaacaacttggactccaaagtgggtggcaactacaactatctgtataggctgtttcgaaagtccaacttgaaaccgtttgaaagggatattagtacagagatctatcaagctggtagcacaccctgcaacggtgttaaaggctttaactgctactttcctttgcagagctatggatttcagcctacttatggagtcggttaccaaccttatcgcgtcgtagtactgagtttcgaactccttcatgcacccgcaactgtgtgcgggccaaagaagtctaccaatttggtcaagaataagtgtgtaaacttcggtagtggttctggtagttggtatatatggctagggttcattgctggacttatagcaattgtgatggtaacgatcatgttgtgttgcatgacttcctgctgcagttgcctcaaaggatgttgttcctgtggatcatgctgtggatctggatccggatcaggagaaggtcgtggttctctacttacgtgcggagatgttgaggagaatccaggacctatgtttgtctttctcgtacttctgcctctggtcagcagccaatgtgtgcgagtgcagcccacagaatccatagttcgattccctaacatcactaatttgtgtccatttggtgaagtgtttaacgcgaccaggttcgccagcgtttatgcatggaatagaaagcggatcagtaattgcgtggccgattattctgtcctgtataattcagcatccttctctactttcaagtgttatggtgtttcaccaaccaaattgaacgatctctgcttcactaatgtttatgcagacagctttgtgatacgaggagatgaggtcagacagattgctcccgggcaaacgggaaagattgctgattataactacaagctgcctgacgactttacgggctgtgtcatagcgtggaacagcaataacctcgatagtaaggtaggcggtaattacaactaccggtatagactgtttcggaagtccaatctgaagcctttcgaacgcgatattagtaccgagatctatcaggcaggttccacaccttgcaatggagtggagggctttaattgctatttccctctgcaatcctacggattccaaccaacctatggcgttggctaccagccttatcgggtggtcgtgctttcctttgaacttcttcacgcacccgctactgtatgtggccctaagaagagcactaaccttgtgaagaacaaatgcgtcaatttcggatcaggaagtggatcatggtacatatggctagggtttatagcaggccttatagcaatcgtcatggttaccatcatgctttgctgtatgaccagttgctgctcatgcctgaagggttgttgttcctgcggatcttgctgttaa

SEQ ID NO: 54 - protein, 909 aa, synthetic construct
  REGION 1..909: note = encoded protein sequence of the RBD domain of the Wuhan-Hu-1 reference strain, the RBD domain of the B.1.351 variant, and an RBD domain combining N501Y and L452R mutations of the B.1.1.7 and B.1.429+B.1.427 variants
  REGION 1..16: note = MISC_FEATURE - signal peptide of S antigen
  REGION 240..245: note = MISC_FEATURE - GS linker
  REGION 287..292: note = MISC_FEATURE - GS linker
  REGION 313..328: note = MISC_FEATURE - signal peptide of S antigen
  REGION 552..557: note = MISC_FEATURE - GS linker
  REGION 599..604: note = MISC_FEATURE - GS linker
  REGION 624..639: note = MISC_FEATURE - signal peptide of S antigen
  REGION 863..868: note = MISC_FEATURE - GS linker
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSWYIWLGFIAGLIAIVMVTIMLCCMTSCCSCLKGCCSCGSCCGSGSGSGATNFSLLKQAGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGSGSWYIWLGFIAGLIAIVMVTIMLCCMTSCCSCLKGCCSCGSCCGSGSGSGEGRGSLLTCGDVEENPGPMFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTYGVGYQPYRVVVLSFELLHAPATVCGPKKSTNLVKNKCVNFGSGSGSWYIWLGFIAGLIAIVMVTIMLCCMTSCCSCLKGCCSCGSCC

SEQ ID NO: 55 - DNA, 1260 bp, synthetic construct
  misc_feature 1..1260: note = ORF of a codon-optimized N antigen that includes the N-specific mutations in the B.1.1.7 variant
  misc_feature 3: note = leucine
  misc_feature 203: note = lysine
  misc_feature 204: note = arginine
  misc_feature 235: note = phenylalanine
  source 1..1260: mol_type = other DNA
atgtctctaaacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaagatgttcggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 56 - protein, 419 aa, synthetic construct
  REGION 1..419: note = encoded protein sequence of a codon-optimized N antigen that includes the N-specific mutations in the B.1.1.7 variant
  SITE 3: note = MISC_FEATURE - leucine
  SITE 203: note = MISC_FEATURE - lysine
  SITE 204: note = MISC_FEATURE - arginine
  SITE 235: note = MISC_FEATURE - phenylalanine
MSLNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMFGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 57 - DNA, 1260 bp, synthetic construct
  misc_feature 1..1260: note = ORF of a codon-optimized N antigen that includes the N-specific mutations in the B.1.351 variant
  misc_feature 205: note = isoleucine
  source 1..1260: mol_type = other DNA
atgtctgataacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtagaggaatctctcctgctagaatggctggcaatggcggtgatgctgctcttgctttgctgctgcttgacagattgaaccagcttgagagcaagatgtctggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 58 - protein, 419 aa, synthetic construct
  REGION 1..419: note = encoded protein sequence of a codon-optimized N antigen that includes the N-specific mutations in the B.1.351 variant
  SITE 205: note = MISC_FEATURE - isoleucine
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGISPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 59 - DNA, 1260 bp, synthetic construct
  misc_feature 1..1260: note = ORF of a codon-optimized N antigen that includes the N-specific mutations in the P.1 variant
  misc_feature 80: note = arginine
  source 1..1260: mol_type = other DNA
atgtctgataacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtcgagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtaaacgaacttctcctgctagaatggctggcaatggcggtgatgctgctcttgctttgctgctgcttgacagattgaaccagcttgagagcaagatgtctggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 60 - protein, 419 aa, synthetic construct
  REGION 1..419: note = encoded protein sequence of a codon-optimized N antigen that includes the N-specific mutations in the P.1 variant
  SITE 80: note = MISC_FEATURE - arginine
MSDNGPQNQRNAPRITFGGPSDSTGSNQNGERSGARSKQRRPQGLPNNTASWFTALTQHGKEDLKFPRGQGVPINTNSSRDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 61 - DNA, 3822 bp, synthetic construct
  misc_feature 1..3822: note = ORF of the codon-optimized SARS-CoV-2 S antigen based on the Wuhan-Hu-1 reference strain
  source 1..3822: mol_type = other DNA
atgtttgtctttcttgtcttattgccactagtctctagtcagtgtgttaatcttacaaccagaactcaattaccacctgcatacactaattctttcacacgtggtgtttattaccctgacaaagtattcagatcctcagtattacattcaactcaggacttgttcttacctttcttctccaatgttacttggttccatgctatacatgtctctgggaccaatggtactaagaggtttgataaccctgtcctaccatttaatgatggtgtttatttcgcttccactgagaagtctaacataataagaggctggatatttggtactactttagattcgaagacccagtccctacttattgttaataacgctactaatgttgttattaaagtctgtgaatttcaattctgtaatgatccattcttggatgtttattaccacaagaacaacaagagttggatgaagagtgagttcagagtttattctagtgcgaataattgcactttcgaatatgtctctcagcctttccttatggaccttgaaggtaaacagggtaatttcaagaatcttagggaatttgtgtttaagaatattgatggttatttcaagatatattctaagcacacgcctattaatttagtgcgtgatctccctcagggtttctcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggactttcctattgaaatataatgagaatggaaccattacagatgctgtagactgtgcacttgaccctctctcagaaacaaagtgtacgttgaaatccttcactgtagagaaaggaatctatcaaacttctaactttagagtccaaccaacagaatctattgttagatttcctaatattacaaacttgtgccctttcggtgaagtatttaacgccaccagatttgcatctgtttatgcttggaacaggaagagaatcagcaactgtgttgctgattattctgtcctatataattccgcatcattctccacatttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgactttacaggctgcgttatagcttggaattctaacaatcttgattctaaggttggtggtaattataattaccggtatagattgtttaggaagtctaatctcaaacctttcgagagagatatttcaactgaaatctatcaggccggtagcacaccttgtaatggtgttcaaggatttaattgttactttcctttacaatcatatggtttccaacccactaatggtgttggttaccaaccatacagagtagtagtactttcatttgaacttctacatgcaccagcaactgtttgtggacctaagaagtctactaatttggttaagaacaaatgtgtcaatttcaacttcaatggtttaacaggcacaggtgttcttactgagtctaacaagaagtttctgcctttccaacaatttggcagagacattgctgacactactgatgctgtccgtgatccacagacacttgagattcttgacattacaccatgttcatttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtatttcaaacacgtgcaggctgtttaataggagctgaacatgtcaacaactcatatgagtgtgacatacccattggtgcaggtatatgcgctagttatcagactcagactaattctcgtcggcgggcacgtagtgtagctagtcaatccatcattgcctacactatgtcacttggtgcagagaattcagttgcttactctaataactctattgccatacccacaaactttactattagtgttaccacagaaattctaccagtgtctatgaccaagacatcagtagattgtacaatgtacatttgtggtgattcaactgaatgcagcaatctattgttgcaatatggcagtttctgtacacaattaaaccgtgctttaactggaatagctgttgaacaagacaagaacacccaagaagtatttgcacaagtcaaacaaatttacaagacaccaccaattaaagatttcggtggatttaatttctcacaaatattaccagatccatctaaaccaagcaagaggtcatttattgaagatctactattcaacaaagtgacacttgcagatgctggcttcatcaaacaatatggtgattgccttggtgatattgctgctagagacctcatttgtgcacagaagtttaacggccttactgtattgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccagaaattgattgccaaccaatttaatagtgctattggcaagattcaagactcactttcttccacagcaagtgcacttggtaaacttcaagatgtggtcaaccagaatgcacaagctttaaacacgcttgttaaacaacttagctccaactttggtgcaatttcaagtgtattaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaagatgtcagagtgtgtacttggacaatctaagagagttgatttctgtggaaagggctatcatcttatgtccttccctcagtcagcacctcatggtgtagtcttcttgcatgtgacttatgtccctgcacacgagaagaacttcacaactgctcctgccatttgtcatgatggtaaagcacactttcctcgtgaaggtgtctttgtttcaaatggcacagactggtttgtaacacaaaggaatttctatgaaccacaaatcattactacagacaacacatttgtgtctggtaactgtgatgttgtaataggaattgtcaacaacacagtttatgatcctttgcaacctgaattagactcattcaaggaggagttagataaatatttcaagaatcatacatcaccagatgttgatttaggtgacatctctggcattaatgcttcagttgtaaacattcagaaagaaattgaccgcctcaatgaggttgccaagaatttaaatgaatctctcatcgatctccaagaacttggaaagtatgagcagtatatcaaatggccatggtacatttggctaggtttcatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 62 - protein, 1273 aa, synthetic construct
  REGION 1..1273: note = encoded protein sequence of the codon-optimized SARS-CoV-2 S antigen sequence based on the Wuhan-Hu-1 reference strain
  REGION 1..16: note = MISC_FEATURE - signal peptide of S antigen
  SITE 142: note = MISC_FEATURE - aspartic acid
  SITE 154: note = MISC_FEATURE - lysine
  SITE 681: note = MISC_FEATURE - lysine
  SITE 1071: note = MISC_FEATURE - histidine
  SITE 1101: note = MISC_FEATURE - histidine aspartic acid
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMKSEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAHEKNFTTAPAICHDGKAHFPREGVFVSNGTDWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 63 - DNA, 1260 bp, synthetic construct
  misc_feature 1..1260: note = ORF of the codon-optimized SARS-CoV-2 N antigen of B.1.617 variant
  source 1..1260: mol_type = other DNA
atgtctgataacggaccacagaatcagcgaaacgcaccacgcattacgtttggtggaccctcagattcaactggcagtaaccagaatggagaacgcagtggagcgcgatctaaacaacgtcggcctcaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaagatgaaagatctcagtccaagatggtatttctactacctaggaactgggccagaagctggacttccctatggtgctaacaaagacggcatcatatgggttgcaactgagggagccttgaatacacctaaagatcacattggcacccgcaatcctgctaacaatgctgcaatcgtgctacaacttcctcaaggaacaacattgcctaaaggcttctacgcagaagggagcagaggcggcagtcaagcctcttctcgttcctcatcacgtagtcgcaacagttcaagaaattcaactccaggcagcagtatgggaacttctcctgctagaatggctggcaatggcggtgatgctgctcttgctttgctgctgcttgacagattgaaccagcttgagagcaagatgtctggtaaaggccaacaacaacaaggccaaactgtcactaagaaatctgctgctgaggcttctaagaagcctcggcagaaacgtactgccactaaagcatacaatgtaacacaagctttcggcagacgtggtccagaacaaacccaaggaaactttggagaccaggaactaatcagacaaggaactgattacaaacattggccgcaaattgcacaatttgctcccagcgcttcagcgttcttcggaatgtcgcgcattggcatggaagtcacaccttcgggaacgtggttgacctacacaggtgccatcaaattggatgacaaagatccaaatttcaaagatcaagtcatattgctgaataagcatattgacgcatacaagacattcccaccaacagagcctaagaaggacaagaagaagaaggcttatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 64 - protein, 419 aa, synthetic construct
  REGION 1..419: note = encoded protein sequence of the codon-optimized SARS-CoV-2 N antigen sequence of the B.1.617 variant
  SITE 203: note = MISC_FEATURE - methionine
  SITE 377: note = MISC_FEATURE - tyrosine
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSMGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKAYETQALPQRQKKQQTVTLLPAADLDDFSKQLQQSMSSADSTQA

SEQ ID NO: 65 - protein, 19 aa, synthetic construct
  REGION 1..19: note = SARS-CoV-2 S antigen
KFDEDDSEPVLKGVKLHYT

SEQ ID NO: 66 - DNA, 215 bp, synthetic construct
  misc_feature 1..215: note = CR/HL/CR
  misc_feature 1..20: note = CR/HL/CR
  misc_feature 25..190: note = duplex copy of MVA terminal HL
  misc_feature 196..215: note = CR/HL/CR
  source 1..215: mol_type = other DNA
tttttttctagacactaaataaatagtaagattaaattaattataaaattatgtatataatattaattataaaattatgtatatgatttactaactttagttagataaattaataatacataaattttagtatattaatattataaattaataatacataaattttagtatattaatattatattttaaatatttatttagtgtctagaaaaaaa

SEQ ID NO: 67 - skipped

SEQ ID NO: 68 - DNA, 73 bp, synthetic construct
  misc_feature 1..73: note = primer
  source 1..73: mol_type = other DNA
aaaaaatatattatttttatgttattttgttaaaaataatcatcgaatacgaactagtataaaaaggcgcgcc

SEQ ID NO: 69 - DNA, 77 bp, synthetic construct
  misc_feature 1..77: note = primer
  source 1..77: mol_type = other DNA
gaagataccaaaatagtaaagattttgctattcagtggactggatgattcaaaaattgaaaataaatacaaaggttc

SEQ ID NO: 70 - DNA, 80 bp, synthetic construct
  misc_feature 1..80: note = primer
  source 1..80: mol_type = other DNA
atatgaatatgatttcagatactatatttgttcctgtagataataactaaaaatttttatctagtataaaaaggcgcgcc

SEQ ID NO: 71 - DNA, 80 bp, synthetic construct
  misc_feature 1..80: note = primer
  source 1..80: mol_type = other DNA
ggaaaatttttcatctctaaaaaaagatgtggtcattagagtttgatttttataaaaattgaaaataaatacaaaggttc

SEQ ID NO: 72 - DNA, 79 bp, synthetic construct
  misc_feature 1..79: note = primer
  source 1..79: mol_type = other DNA
ttggggaaatatgaacctgacatgattaagattgctctttcggtggctggtaaaaaattgaaaataaatacaaaggttc

SEQ ID NO: 73 - DNA, 70 bp, synthetic construct
  misc_feature 1..70: note = primer
  source 1..70: mol_type = other DNA
acaaaattatgtattttgttctatcaactacctataaaactttccaaatactagtataaaaaggcgcgcc

SEQ ID NO: 74 - DNA, 44 bp, synthetic construct
  misc_feature 1..44: note = primer
  misc_feature 21: note = n is a, c, g, or t
  source 1..44: mol_type = other DNA
tcgtggtgtgcctgaatcgandaggtagcgacttcaggtttctt

SEQ ID NO: 75 - DNA, 43 bp, synthetic construct
  misc_feature 1..43: note = primer
  misc_feature 22: note = n is a, c, g, or t
  source 1..43: mol_type = other DNA
tatccaccaatccgagaccaandcctctggaccgcataatctg

SEQ ID NO: 76 - DNA, 51 bp, synthetic construct
  misc_feature 1..51: note = primer
  misc_feature 26: note = n is a, c, g, or t
  source 1..51: mol_type = other DNA
aggtttgatcgttgtcatttctccandagagggatattaagtcgatagccg

SEQ ID NO: 77 - DNA, 19 bp, synthetic construct
  misc_feature 1..19: note = primer
  source 1..19: mol_type = other DNA
tggaatgcgttccttgtgc

SEQ ID NO: 78 - DNA, 21 bp, synthetic construct
  misc_feature 1..21: note = primer
  source 1..21: mol_type = other DNA
cgtttttcccattcgatacag

SEQ ID NO: 79 - DNA, 23 bp, synthetic construct
  misc_feature 1..23: note = primer
  source 1..23: mol_type = other DNA
tatagtctttgtggcatccgttg

SEQ ID NO: 80 - DNA, 23 bp, synthetic construct
  misc_feature 1..23: note = primer
  source 1..23: mol_type = other DNA
acccaaactttagtaaggccatg

SEQ ID NO: 81 - DNA, 20 bp, synthetic construct
  misc_feature 1..20: note = primer
  source 1..20: mol_type = other DNA
ataagcgttgtcaaagcggg

SEQ ID NO: 82 - DNA, 23 bp, synthetic construct
  misc_feature 1..23: note = primer
  source 1..23: mol_type = other DNA
aggaaatagaaattgttggtgcg

SEQ ID NO: 83 - DNA, 23 bp, synthetic construct
  misc_feature 1..23: note = primer
  source 1..23: mol_type = other DNA
acattggcggacaatctaaaaac

SEQ ID NO: 84 - DNA, 25 bp, synthetic construct
  misc_feature 1..25: note = primer
  source 1..25: mol_type = other DNA
atcatcggtggttgatttagtagtg

SEQ ID NO: 85 - DNA, 20 bp, synthetic construct
  misc_feature 1..20: note = primer
  source 1..20: mol_type = other DNA
tatccaccaatccgagacca

SEQ ID NO: 86 - DNA, 25 bp, synthetic construct
  misc_feature 1..25: note = primer
  source 1..25: mol_type = other DNA
gtctgtccgtcttctctattgttta

SEQ ID NO: 87 - DNA, 25 bp, synthetic construct
  misc_feature 1..25: note = primer
  source 1..25: mol_type = other DNA
ttaactcagtttcaatacggtgcag

SEQ ID NO: 88 - DNA, 22 bp, synthetic construct
  misc_feature 1..22: note = primer
  source 1..22: mol_type = other DNA
tggggtttcttctcaggctatc

SEQ ID NO: 89 - protein, 15 aa, synthetic construct
  REGION 1..15: note = peptide N26
MKDLSPRWYFYYLGT

SEQ ID NO: 90 - protein, 9 aa, synthetic construct
  REGION 1..9: note = HLA-B*0702 immunodominant N-specific peptide epitope
SPRWYFYYL